(19) United States
(12) Patent Application Publication
(10) Pub. No.: US 2022/0243263 A1
(43) Pub. Date: Aug. 4, 2022

(54) MARKERS FOR IDENTIFYING AND QUANTIFYING OF NUCLEIC ACID SEQUENCE MUTATION, EXPRESSION, SPLICE VARIANT, TRANSLOCATION, COPY NUMBER, OR METHYLATION CHANGES

(71) Applicant: CORNELL UNIVERSITY, Ithaca, NY (US)

(72) Inventors: Francis BARANY, New York, NY (US); Manny D. BACOLOD, New York, NY (US); Jianmin HUANG, New York, NY (US); Aashiq H. MIRZA, New York, NY (US); Philip B. FEINBERG, New York, NY (US); Sarah F. GIARDINA, New York, NY (US)

(21) Appl. No.: 17/608,537

(22) PCT Filed: May 1, 2020
(86) PCT No.: PCT/US20/31044

§ 371 Date: Nov. 3, 2021

Related U.S. Application Data
(60) Provisional application No. 62/843,032, filed on May 3, 2019.

(51) Int. Cl. C12Q 1/6858; C12Q 1/6886

(52) U.S. Cl. CPC C12Q 1/6858; C12Q 1/6886; C12Q 2600/178; C12Q 2600/156; C12Q 2600/158; C12Q 2600/154

(57) ABSTRACT

The present invention relates to methods for identifying and/or quantifying low abundance, nucleotide base mutations, insertions, deletions, translocations, splice variants, miRNA variants, alternative transcripts, alternative start sites, alternative coding sequences, alternative non-coding sequences, alternative splicings, exon insertions, exon deletions, intron insertions, or other rearrangement at the genome level and/or methylated nucleotide bases.

This application claims benefit of U.S. Provisional Patent Application Ser. No. 62/843,032, filed May 3, 2019, which is hereby incorporated by reference in its entirety.

This invention was made with government support under grant number P41 EB020594 awarded by National Institutes of Health. The government has certain rights in the invention.

### FIELD

The present application relates to methods and markers for identifying and quantifying nucleic acid sequence, mutation, expression, splice variant, translocation, copy number, and/or methylation changes using combinations of bisulfite treatment, nuclease, ligation, and polymerase reactions with carryover prevention.

### BACKGROUND

Cancer is the leading cause of death in developed countries and the second leading cause of death in developing countries. Cancer kills 580,000 patients annually in the US, 1.3 million in Europe, and 2.8 million in China (Siegel et al., “Cancer Statistics, 2016,” CA Cancer J. Clin. 66(1):7-30 (2016)). Cancer is now the biggest cause of mortality worldwide, with an estimated 8.2 million deaths from cancer in 2012 (Torre et al., “Global Cancer Statistics, 2012,” CA Cancer J. Clin. 65(2):87-108 (2015)). Cancer cases worldwide are forecast to rise by 75% and reach close to 25 million over the next two decades. The lifetime risk of a woman dying from an invasive cancer is 19%, for a man it is 23%. With total annual costs of cancer care in the U.S. exceeding $400 billion, there is no other medical issue that so urgently needs intelligent solutions.

In the U.S., new cancer cases among men are dominated by prostate (21%), lung (14%), colorectal (8%), urinary bladder (7%), melanoma (6%), non-Hodgkin lymphoma (5%), renal (5%), head and neck (4%), leukemia (4%), and liver and bile cancer (3%). Among women, most of the newly diagnosed cancers are breast (29%), lung (13%), colorectal (8%), uterine corpus (7%), thyroid (6%), non-Hodgkin lymphoma (4%), melanoma (3%), leukemia (3%), pancreatic (3%), and renal cancer (3%). The leading causes of cancer deaths are lung cancer (27%), prostate cancer (8%), colorectal cancer (8%), and lung cancer (26%), breast cancer (14%), colorectal cancer (8%), for men and women, respectively. These cancers are driven by different biological processes, and while there have been exciting advancements in the treatment of some cancers, such as the emergence of targeted therapeutics and immunotherapy, most cancers are found at later stage, where survival is poor. Due to lack of reliable and inexpensive early detection tests, many cancer types are diagnosed at later stages, where survival rates for some cancers drop to below 10%. The current screening technologies are failing due to low patient compliance, high expense, and low sensitivity and specificity rates (Das et al., “Predictive and Prognostic Biomarkers in Colorectal Cancer: A Systematic Review of Recent Advances and Challenges,” Biomedicine & Pharmacotherapy 87:8-19 (2016)). For example, the high cost, discomfort, and invasiveness of colonoscopy are significant impediments to patient compliance for CRC screening (Beydoun et al., “Predictors of Colorectal Cancer Screening Behaviors Among Average-risk Older Adults in the United States,” Cancer Causes & Control: CCC 19(4):339-359 (2008)). Likewise, patient distaste for handling feces has limited the success of FOBT/FIT, and eliminated stool-based tests as a remedy for low compliance. In contrast, the current proposal addresses these problems by developing a blood test with the potential to become widely adopted. Increasing patient compliance for CRC testing will lead to earlier detection and, ultimately, increased patient survival.

Ultimately, there is an urgent need to develop non-invasive, highly sensitive, highly specific, and cost-effective tests which will detect early-stage cancers. Two relatively recent developments in cancer research serve as the guiding principles for these tasks. First, is the use of modern genomic tools (such as genome-wide sequencing, transcriptional, and methylation profiling). Public accessibility to vast databases generated from these studies has accelerated the discovery of a wider list of molecular markers (such as promoter methylation, mutation, copy number, or expression levels of mRNA, microRNA, non-coding RNA (ncRNA), and long non-coding RNA (lncRNA) associated with cancer progression. Second is the discovery that nucleic acids can be released by the cancer cells into the patient's bloodstream. Cancer cells may undergo apoptosis (triggered cell death), which releases cell free DNA (cfDNA) into the patients' blood (Salvi et al., “Cell-free DNA as a Diagnostic Marker for Cancer: Current Insights,” OncoTargets and Therapy 9:6549-6559 (2016)). The levels of cfDNA in serum from patients with cancer vary from vanishingly small to high, but do not correlate with cancer stage (Perlin et al., “Serum DNA Levels in Patients With Malignant Disease,” American Journal of Clinical Pathology 58(5):601-602 (1972); Leon et al., “Free DNA in the Serum of Cancer Patients and the Effect of Therapy,” Cancer Res. 37(3):646-650 (1977)). Moreover, exosomes (lipid vesicles ranging from 30 to 100 nm), which are released into the blood by cancer cells, can contain the same RNA molecules which serve as transcriptional signatures of the tumors. Exosomes, or tumor associated vesicles, shield mRNA, lncRNA, ncRNA, and even mutant tumor DNA from exogenous nucleases, and, as such, the markers are in a protected state. Other protected states include, but are not limited to, DNA, RNA, and proteins within circulating tumor cells (CTCs), within other non-cellular membrane containing vesicles or particles, within nucleosomes, or within Argonaute or other protein complexes. cfDNA in particular, contains the same molecular aberrations as the solid tumors, such as mutations hyper/hypo methylation, copy number changes, or chromosomal rearrangements (Ignatiadis et al., “Circulating Tumor Cells and Circulating Tumor DNA for Precision Medicine: Dream or Reality?” Ann. Oncol. 25(12):2304-2313 (2014)).

Tumor-specific CpG methylations have been detected in the plasma from patients with a variety of solid tumors (Pratt V M, “Are We Ready for a Blood-Based Test to Detect Colon Cancer?” Clinical Chemistry 60(9):1141-1142 (2014); Warton et al., “Methylation of Cell-free Circulating DNA in the Diagnosis of Cancer,” Frontiers in Molecular Biosciences 2:13 (2015)), through various techniques involving bisulfite conversion of unmethylated cytosines, methylation-sensitive enzymes, or immunoprecipitation of 5-methylcytosines (Jorda et al., “Methods for DNA methylation analysis and applications in colon cancer,” Mutat. Res. 693(1-2):84-93 (2010)). Methylation signatures have better specificity towards a particular cancer type likely because methylation patterns are highly tissue specific (Issa J P, “DNA Methylation as a Therapeutic Target in Cancer,” Clin. Cancer Res. 13(6):1634-1637 (2007)). The best studied blood-based methylation markers for CRC detection are located in the promoter region of the SEPT9 gene (Church et al., “Prospective Evaluation of Methylated SEPT9 in Plasma for Detection of Asymptomatic Colorectal Cancer,” Gut 63(2):317-325 (2014); Lofton-Day et al., “DNA Methylation Biomarkers for Blood-Based Colorectal Cancer Screening,” Clinical Chemistry 54(2):414-423 (2008); Potter et al., “Validation of a Real-time PCR-based Qualitative Assay for the Detection of Methylated SEPT9 DNA in Human Plasma,” Clinical Chemistry 60(9):1183-1191 (2014); Ravegnini et al., “Simultaneous Analysis of SEPT9 Promoter Methylation Status, Micronuclei Frequency, and Folate-Related Gene Polymorphisms: The Potential for a Novel Blood-Based Colorectal Cancer Biomarker,” International Journal of Molecular Sciences 16(12):28486-28497 (2015); Toth et al., “Detection of Methylated SEPT9 in Plasma is a Reliable Screening Method for Both Left- and Right-sided Colon Cancers,” PloS One 7(9):e46000 (2002); Toth et al., “Detection of Methylated Septin 9 in Tissue and Plasma of Colorectal Patients with Neoplasia and the Relationship to the Amount of Circulating Cell-Free DNA,” PloS One 9(12):e115415 (2014); Warren et al., “Septin 9 Methylated DNA is a Sensitive and Specific Blood Test for Colorectal Cancer,” BMC Medicine 9:133 (2011)), and other potential markers for CRC diagnostics include CpG sites on promoter regions of THBD (Lange et al., “Genome-scale Discovery of DNA-methylation Biomarkers for Blood-Based Detection of Colorectal Cancer,” PloS One 7(11):e50266 (2012)), C9orf50 (Lange et al., “Genome-scale Discovery of DNA-methylation Biomarkers for Blood-Based Detection of Colorectal Cancer,” PloS One 7(11):e50266 (2012)), ZNF154 (Margolin et al., “Robust Detection of DNA Hypermethylation of ZNF154 as a Pan-Cancer Locus with in Silico Modeling for Blood-Based Diagnostic Development,” The Journal of Molecular Diagnostics 18(2):283-298 (2016)), and AGBL4, FLI1 and TWIST1 (Lin et al., “Clinical Relevance of Plasma DNA Methylation in Colorectal Cancer Patients Identified by Using a Genome-Wide High-Resolution Array,” Ann. Surg. Oncol. 22 Suppl 3:S1419-1427 (2015)). In breast cancer, methylation at promoter regions of tumor suppressor genes (including ATM, BRCA1, RASSF1, APC, and RARβ) has been detected in patients' cfDNAs (Tang et al., “Blood-based DNA Methylation as Biomarker for Breast Cancer: a Systematic Review,” Clinical Epigenetics 8:115 (2016)). A caveat for using methylation markers is that bisulfite conversion tends to destroy DNA, and thus decreases the overall signal that can be detected. Methylation detection techniques may also lead to false-positive signals due to incomplete conversion of unmethylated cytosines. As described herein, an extensive bioinformatics analysis of public databases has been performed to identify CRC-specific, and tissues-specific methylation markers suitable for detection of cancer in the plasma. The methylation marker detection assays enable a higher level of multiplexing with single-molecule detection capabilities, which are predicted to allow for higher sensitivity and specificity across a broad spectrum of cancers.

The challenge to develop reliable diagnostic and screening tests is to distinguish those markers emanating from the tumor that are indicative of disease (e.g., early cancer) vs. presence of the same markers emanating from normal tissue (which would lead to a false-positive signal). There is also a need to balance the number of markers examined and the cost of the test, with the specificity and sensitivity of the assay. Comprehensive molecular profiling (mRNA, methylation, copy number, miRNA, mutations) of thousands of tumors by The Cancer Genome Atlas Consortium (TCGA), has revealed that colorectal tumors are as different from each other as they are from breast, prostrate, or other epithelial cancers (TCGA “Comprehensive Molecular Characterization of Human Colon and Rectal Cancer Nature 487:330-337 (2014)). Further, those few markers they share in common are also present in multiple cancer types, hindering the ability to pinpoint the tissue of origin. BRAF mutations frequently occur in melanoma (42%) and thyroid cancer (41%), while KRAS is also highly mutated in pancreatic (55%) and lung (16%) cancers (Forbes et al., “COSMIC: Exploring the World's Knowledge of Somatic Mutations in Human Cancer,” Nucleic Acids Res. 43(Database issue):D805-811 (2015)). In general, CRC mutation markers such as those of KRAS and BRAF are found in late-stage primary cancers and metastases (Spindler et al., “Circulating free DNA as Biomarker and Source for Mutation Detection in Metastatic Colorectal Cancer,” PloS One 10(4):e0108247 (2015); Gonzalez-Cao et al., “BRAF Mutation Analysis in Circulating Free Tumor DNA of Melanoma Patients Treated with BRAF Inhibitors,” Melanoma Res. 25(6):486-495 (2015); Sakai et al., “Extended RAS and BRAF Mutation Analysis Using Next-Generation Sequencing,” PloS One 10(5):e0121891 (2015)). For early cancer detection, the nucleic acid assay should serve primarily as a screening tool, requiring the availability of secondary diagnostic follow-up (e.g., colonoscopy for colorectal cancer).

Compounding the biological problem is the need to reliably quantify mutation, CpG methylation, or DNA or RNA copy number from either a very small number of initial cells (i.e. from CTCs), or when the cancer signal is from cell-free DNA (cfDNA) in the blood and diluted by an excess of nucleic acid arising from normal cells, or inadvertently released from normal blood cells during sample processing (Mateo et al., “The Promise of Circulating Tumor Cell Analysis in Cancer Management,” Genome Biol. 15:448 (2014); Haque et al., “Challenges in Using ctDNA to Achieve Early Detection of Cancer,” BioRxiv. 237578 (2017)).

Some cancer IVD companies have developed commercially available methylation detection tests. The aforementioned SEPT9 methylation is the basis for Epi proColon test, a CRC-detection assay by Epigenomics (Lofton-Day et al., “DNA Methylation Biomarkers for Blood-based Colorectal Cancer Screening,” Clinical Chemistry 54(2):414-423 (2008)). While initial results on smaller sample sets showed promise, large-scale studies with 1,544 plasma samples showed a sensitivity of 64% for stage I-III CRC, and a specificity of 78%-82%, effectively sending 180 to 220 out of 1,000 individuals to unnecessary colonoscopies (Potter et al., “Validation of a Real-time PCR-based Qualitative Assay for the Detection of Methylated SEPT9 DNA in Human Plasma,” Clinical Chemistry 60(9):1183-1191 (2014)). Clinical Genomics is currently developing blood based CRC detection test based on the methylation of the BCAT1 and IKZF1 genes (Pedersen et al., “Evaluation of an Assay for Methylated BCAT1 and IKZF1 in Pasma for Detection of Colorectal Neoplasia,” BMC Cancer 15:654 (2015)]. Large-scale studies using 2,105 plasma samples of this two-marker test showed an overall sensitivity of 66%, with 38% for stage I CRC, and an impressive specificity of 94% (Young et al, “A Cross-sectional Study Comparing a Blood Test for Methylated BCAT1 and IKZF1 Tumor-derived DNA with CEA for Detection of Recurrent Colorectal Cancer,” Cancer Medicine 5(10): 2763-2772 (2016)). Exact Sciences and collaborators have slightly improved the sensitivity of CRC fecal tests (Bosch et al., “Analytical Sensitivity and Stability of DNA Methylation Testing in Stool Samples for Colorectal Cancer Detection,” Cell Oncol. (Dordr) 35(4):309-315 (2012); Hong et al., “DNA Methylation Biomarkers of Stool and Blood for Early Detection of Colon Cancer,” Genet. Test. Mol. Biomarkers 17(5):401-406 (2013); Imperiale et al., “Multitarget Stool DNA Testing for Colorectal-Cancer Screening,” N. Engl. J. Med. 370(14):1287-1297 (2014); Xiao et al., “Validation of Methylation-Sensitive High-Resolution Melting (MS-HRM) for the Detection of Stool DNA Methylation in Colorectal Neoplasms,” Clin. Chim. Acta 431:154-163 (2014); Yang et al., “Diagnostic Value of Stool DNA Testing for Multiple Markers of Colorectal Cancer and Advanced Adenoma: a Meta-Analysis,” Can. J. Gastroenterol. 27(8):467-475 (2013)), by adding K-ras mutation as well as BMP3 and NDRG4 methylation markers (Lidgard et al., “Clinical Performance of an Automated Stool DNA Assay for Detection of Colorectal Neoplasia,” Clin. Gastroenterol. Hepatol. 11(10):1313-1318 (2013)). Large-scale studies on 12,500 stool samples claims 93% sensitivity, yet specificity is still only 85%, essentially sending 150 out of 1,000 individuals to unnecessary colonoscopies. Despite logistical issues in handling feces, Exact Sciences recently sold their millionth test. The Cologuard website states the test result has both false-positives and false-negatives, and the test should not be used if the patient has hemorrhoids, menstrual period, or blood in the stool. The Cologuard website also warns that the test is not for use by patients with Ulcerative Colitis (UC), Crohn's disease (CD), Inflammatory Bowel Disease (IBD), or with a family history of cancer. In other words, Exact Sciences excludes the very patients who would most benefit from an accurate CRC detection test. More recently, Laboratory for Advanced Medicine (based in Irvine, Calif. with ties to various Chinese academic institutions) demonstrated the potential of interrogating the methylation status of a single CpG site (cg10673833) for blood-based detection of colorectal cancer (Luo et al., “Circulating Tumor DNA Methylation Profiles Enable Early Diagnosis, Prognosis Prediction, and Screening for Colorectal Cancer,” Science Translational Medicine 12:(524) (2020)).

### A Continuum of Diagnostic Needs Will Require a Continuum of Diagnostic Tests.

The majority of current molecular diagnostics efforts in cancer have centered on: (i) prognostic and predictive genomics, e.g., identifying inherited mutations in cancer predisposition genes, such as BrCA1, BrCA2, (Ford et al., Am. J. Hum. Genet. 62:676-689 (1998)) (ii) individualized treatment, e.g., mutations in the EGFR gene guiding personalized medicine (Sequist and Lynch, Ann. Rev. Med, 59:429-442 (2008)), and (iii) recurrence monitoring, e.g., detecting emerging KRAS mutations in patients developing resistance to drug treatments (Hiley et al., Genome Biol. 15: 453 (2014); Amado et al., J. Clin. Oncol. 26:1626-1634 (2008)). Yet, this misses major opportunities in the cancer molecular diagnostics continuum: (i) more frequent screening of those with a family history, (ii) screening for detection of early disease, and (iii) monitoring treatment efficacy. To address these three unmet needs, a new metric for blood-based detection termed “cancer marker load”, analogous to viral load is herein proposed.

DNA sequencing provides the ultimate ability to distinguish all nucleic acid changes associated with disease. However, the process still requires multiple up-front sample and template preparation, and consequently, DNA sequencing is not always cost-effective. DNA microarrays can provide substantial information about multiple sequence variants, such as SNPs or different RNA expression levels, and are less costly then sequencing; however, they are less suited for obtaining highly quantitative results, nor for detecting low abundance mutations. On the other end of the spectrum is the TaqMan™ reaction, which provides real-time quantification of a known gene, but is less suitable for distinguishing multiple sequence variants or low abundance mutations.

NGS requires substantial up-front sample preparation to polish ends and append linkers, and the current error rates of 0.7% are too high to identify 2-3 molecules of mutant sequence in a 10,000-fold excess of wild-tye molecules. “Deep sequencing” protocols have been developed to overcome this deficiency by appending unique molecular identifiers to both strands of an individual fragment. These approaches are known as: Tam-Seq & CAPP-Seq (Roche), Circle-Seq (Guardant Health), Safe-SeqS (Personal Genome Diagnostics), ThruPlex (Rubicon Genomics), NEBNext (New England Biolabs), QIAseq (Qiagen), Oncomine (ThermoFisher), Duplex Barcoding (Schmitt), SMRT (Pacific Biosciences), SiMSen-Seq (Stahlberg), and smMIP (Shendure). However, these methods require a 30 to 100-fold depth per mutant strand to verify each mutation and distinguish from other types of sequencing errors. Recent work from MSKCC demonstrates that 60,000-fold coverage is required to accurately identify mutations in plasma from metastatic cancer patients (91% sensitivity, 508-gene panel, 60,000×). Compounding the challenge, a recent paper from NEB has called into question the quality of the most widely used databases for rare variant and somatic mutations (Chen et al., “DNA Damage is a Pervasive Cause of Sequencing Errors, Directly Confounding Variant Identification,” Science 355(6326):752-756 (2017)).

It is critical to match each unmet diagnostic need with the appropriate diagnostic test—one that combines the divergent goals of achieving both high sensitivity (i.e., low false-negatives) and high specificity (i.e., low false-positives) at a low cost. For example, direct sequencing of EGFR exons from a tumor biopsy to determine treatment for non-small cell lung cancer (NSCLC) is significantly more accurate and cost effective than designing TaqMan™ probes for the over 180 known mutations whose drug response is already catalogued (Jia et al., Genome Res. 23:1434-1445 (2013)). The most sensitive technique for detecting point mutations, such as “BEAMing” (Dressman et al., Proc. Natl. Acad. Sci. USA 100: 8817-8822 (2003)), rely on prior knowledge of which mutations to look for, and thus are best suited for monitoring for disease recurrence, rather than for early detection. Likewise, to monitor blood levels of Bcr-Abl translocations when treating CML patients with Gleevec (Jabbour et al., Cancer 112: 2112-2118 (2008)), a simple quantitative reverse-transcription PCR assay is far preferable to sequencing the entire genomic DNA in 1 ml of blood (9 million cells×3 GB=27 million Gb of raw data).

Sequencing 2.1 Gb each of cell-free DNA (cfDNA) isolated from NSCLC patients was used to provide 10,000-fold coverage on 125 kb of targeted DNA (Kandoth et al., Nature 502: 333-339 (2013)). This approach correctly identified mutations present in matched tumors, although only 50% of stage 1 tumors were covered. The approach has promise for NSCLC, where samples average 5 to 20 mutations/Mb, however targeted NGS would not be cost effective for other cancers such as breast and ovarian, that average less than 1 to 2 mutations per Mb. Current up-front ligation, amplification, and/or capture steps required for highly accurate targeted deep sequencing are still more complex than multiplexed PCR-TaqMan™ or PCR-LDR assays.

Deep sequencing of cfDNAs for 58 cancer-related genes at 30,000-fold coverage is capable of detecting Stage 1 or 2 cancer at moderately high sensitivity but missed 29% of CRC, 41% of breast, 41% of lung, and 32% of ovarian cancer, respectively (Phallen et al., “Direct Detection of Early-stage Cancers Using Circulating Tumor DNA,” Science Translational Medicine 9(403) (2017)). An alternative strategy relied on targeted sequencing of an average of 30 bases in 61 segments to interrogate “hot-spot” mutations in 16 genes including TP53, KRAS, APC, PIK3CA, PTEN, missed more early cancers (Cohen et al., “Detection and Localization of Surgically Resectable Cancers with a Multi-analyte Blood Test,” Science (2018). To extend the sensitivity of mutation sequencing, the Hopkins team very recently combined NGS with quantitation of serum protein markers (such as CA-125, CA19-9, CEA, HGF, Myeloperoxidase, OPN, Prolactin, TIMP-1) and improved detection of five cancer types (ovary, liver, stomach, pancreas, and esophagus) at sensitivities ranging from 69% to 98% (Cohen et al. “Detection and Localization of Surgically Resectable Cancers with a Multi-analyte Blood Test,” Science (2018). One caveat of using these protein markers is that prior large-scale studies with age-matched controls (n=22,000) have not shown clinical utility (Jacobs et al., “Prevalence Screening for Ovarian Cancer in Postmenopausal Women by CA 125 Measurement and Ultrasonography,” BMJ 306(6884). 1030-1034 (1993)). Thus, in a 2018 JAMA report, “The USPSTF recommends against [CA-125] screening for ovarian cancer in asymptomatic women. This recommendation applies to asymptomatic women who are not known to have a high-risk hereditary cancer syndrome” (USPSTF et al., “Screening for Ovarian Cancer: US Preventive Services Task Force Recommendation Statement” JAMA 319(6):588-594 (2018)). Another caveat of using these protein markers is that they reflect tissue damage and are likely to also appear in patients with inflammatory diseases such as arthritis (Kaiser, “‘Liquid Biopsy for Cancer Promises Early Detection,” Science 359(6373):259 (2018)). With the growing obesity epidemic and an aging population in the U.S., the risk of false-positives from protein markers increases with obesity and age-driven inflammation.

More recently, the NGS sequencing companies (Grail, Guardant Health, Natera, Freenome) have moved aggressively to expand their targeted sequencing panels to also now include whole genome sequencing (WGS) and whole genome bisulfite sequencing (Bis-WGS). The recent results from Grail, abstract published at ASCO 2018 (Klein et al., “Development of a Comprehensive Cell-free DNA (cfDNA) Assay for Early Detection of Multiple Tumor Types: The Circulating Cell-free Genome Atlas (CCGA) Study,” ASCO Annual Meeting 2018, Chicago, Ill.; Abstract 12021 #134)) reveal that while sensitivity claims of detecting “early” CRC are at 63%, that is based on only 27 samples, most of which are Stage III. Even mutation rich lung cancer gives sensitivity at 50%, again with most samples at Stage III. When most of the samples are Stage I & II, such as prostate cancer, the sensitivity for “early cancer” detection drops to <5%. When attempting to detect the most common form of breast cancer (HR+/HER2), the sensitivity drops to <13%. Worse, those breast cancers diagnosed by screening gave sensitivities of <11%. In short, the NGS approach fails by consistently missing 30% to 80% of early-stage cancers (i.e. stage I & II). In a research initially reported in 2019 ASCO meeting (Liu et al., “Simultaneous Multi-cancer Detection and Tissue of Origin (TOO) Localization Using Targeted Bisulfite Sequencing Plasma Cell-free DNA (cfDNA),” ASCO Breakthrough Presentation 2019)), and subsequently published in 2020 (Liu et al., “Sensitive and Specific Multi-cancer Detection and Localization Using Methylation Signatures in Cell-free DNA,” Annals of Oncology; In Press (2020)), GRAIL indicated that their Multi-Cancer Early Detection Test exhibited an Overall Detection Rate (12 deadly cancer types) of 76% (99.3% specificity). A combined analysis of this group of cancers showed robust detection across all stages with detection rates of 39 percent (27-52%), 69 percent (56-80%), 83 percent (75-90%), and 92 percent (86-96%) at stages I (n=62), II (n=62), III (n=102), and IV (n=130), respectively. In another conference, GRAIL and collaborators (Oxnard et al., “Simultaneous Multi-cancer Detection and Tissue of Origin (TOO) Localization Using Targeted Bisulfite Sequencing of Plasma Cell-free DNA (cfDNA),” ESMO Congress (2019)), reported the results from their analysis of cell-free DNA (DNA that had once been confined to cells but had entered the bloodstream upon the cells' death) in 3,583 blood samples, including 1,530 from patients diagnosed with cancer and 2,053 from people without cancer. The patient samples comprised more than 20 types of cancer, including hormone receptor-negative breast, colorectal, esophageal, gallbladder, gastric, head and neck, lung, lymphoid leukemia, multiple myeloma, ovarian, and pancreatic cancer. The overall specificity was 99.4%, meaning only 0.6% of the results incorrectly indicated that cancer was present. The sensitivity of the assay for detecting a pre-specified high mortality cancer (the percent of blood samples from these patients that tested positive for cancer) was 76%. Within this group, the sensitivity was 32% for patients with stage I cancer; 76% for those with stage II; 85% for stage III; and 93% for stage IV. Sensitivity across all cancer types was 55%, with similar increases in detection by stage. For the 97% of samples that returned a tissue of origin result, the test correctly identified the organ or tissue of origin in 89% of cases. However, another 2019 study (reported by GRAIL and collaborators) questioned the validity of the aforementioned reports (Razavi et al., “High-intensity Sequencing Reveals the Sources of Plasma Circulating Cell-free DNA Variants,” Nat Med 25(12):1928-1937 (2019)). Through a 2 Mb, 508-gene panel sequencing (60,000×depth), the authors demonstrated the vast majority of cell-free DNA mutations in both non-cancer controls and cancer patients had features consistent with clonal hematopoiesis, a process whereby white blood cells progressively accumulate somatic alterations without necessarily producing a hematological condition or malignancy. Indeed. mutations appeared in 93.6 percent of the white blood cells from individuals without cancer and 99.1 percent of those with cancer. In a recently held conference, GRAIL and their collaborators reported that their blood-based test can detect multiple GI cancers at sensitivity of under 50% for Stage I, and 73% for Stage I-III (Wolpin et al., “Performance of a Blood-based Test for the Detection of Multiple Cancer Types,” In: Gastrointestinal Cancers Symposium 2020 (2020)). As for Freenome, a recent ASCO presentation indicated that their platform (plasma analysis by whole-genome sequencing, bisulfite sequencing, and protein quantification methods) was able to achieve a mean sensitivity of 92% in early-stage (n=17) and 84% in late-stage (n=11) at a specificity of 90% for colorectal adenocarcinoma detection. Across all CRC pathological subtypes, the Freenome test achieved a specificity of 90%, and sensitivities of 80% and 83% for early-stage (n=19) and late-stage (n=12), respectively. Private discussion with Imran Haque, who just resigned as CSO of Freenome—where he had a $70 million budget and 30 scientists to sequence the plasma of 817 CRC and matched control patients−confirmed that Freenome (as well as GRAIL) were overcalling the data, and that none of them had a cogent approach to achieve cost-effective true early cancer detection (Wan et al., “Machine Learning Enables Detection of Early-stage Colorectal Cancer by Whole-genome Sequencing of Plasma Cell-free DNA,” BioRxiv 478065 (2018)).

A comprehensive data analysis of over 600 colorectal cancer samples that takes into account tumor heterogeneity, tumor clusters, and biological/technical false-positives ranging from 3% to 10% per individual marker showed that the optimal early detection screen for colorectal cancer would require at least 5 to 6 positive markers out of 24 markers tested (Bacolod et al. Cancer Res. 69:723-727 (2009); Tsafrir et al., Cancer Res. 66: 2129-2137 (2006); Weinstein et al., Nat. Genet. 45: 1113-1120 (2013); Navin N. E. Genome Biol. 15: 452 (2014); Hiley et al., Genome Biol 15:453 (2014)); Esserman et al. Lancet Oncol 15:e234-242 (2014)). Further, marker distribution is biased into different tumor clades, e.g., some tumors are heavily methylated, while others are barely methylated, and indistinguishable from age-related methylation of adjacent tissue. Consequently, a multidimensional approach using combinations of 3-5 sets of mutation, methylation, miRNA, ncRNA, lncRNA, mRNA, copy-variation, alternative splicing, or translocation markers is needed to obtain sufficient coverage of all different tumor clades. Analogous to non-invasive prenatal screening for trisomy, based on sequencing or performing ligation detection on random fragments of cfDNA (Benn et al., Ultrasound Obstet. Gynecol. 42(1):15-33 (2013); Chiu et al., Proc. Natl. Acad. Sci. USA 105: 20458-20463 (2008); Juneau et al., Fetal Diagn. Ther. 36(4) (2014)), the actual markers scored in a cancer screen are secondary to accurate quantification of those positive markers in the plasma.

As pointed out above, cancer-specific RNA markers (including microRNAs, lncRNAs, and mRNAs) may also be present in blood, either free of any compartment (Souza et al., “Circulating mRNAs and miRNAs as Candidate Markers for the Diagnosis and Prognosis of Prostate Cancer,” PloS One 12(9):e0184094 (2017)), or contained in exosomes (Nedaeinia et al., “Circulating Exosomes and Exosomal microRNAs as Biomarkers in Gastrointestinal Cancer,” Cancer Gene Ther 24(2):48-56 (2017); Lai et al., “A microRNA Signature in Circulating Exosomes is Superior to Exosomal Glypican-1 Levels for Diagnosing Pancreatic Cancer,” Cancer Lett 39:86-93 (2017)) or circulating tumor cells (“CTCs”), and have been tagged as potential indicators of early-stage cancers. Challenges abound regarding the use of plasma-derived nucleic markers in early cancer detection, including the minuscule amount of these markers in blood relative to those derived from surrounding cells. Indeed, these limitations make it appear that these “early” detection assays are more likely to detect late-stage primary and metastatic cancers (Pantel “Blood-Based Analysis of Circulating Cell-Free DNA and Tumor Cells for Early Cancer Detection,” PLoS Med 13(12):e1002205 (2016)).

### Technical Challenges of Cancer Diagnostic Test Development.

Diagnostic tests that aim to find very rare or low-abundance mutant sequences face potential false-positive signal arising from: (i) polymerase error in replicating wild-type target, (ii) DNA sequencing error, (iii) mis-ligation on wild-type target, (iii) target independent PCR product, and (iv) carryover contamination of PCR products arising from a previous positive sample. The profound clinical implications of a positive test result when screening for cancer demand that such a test use all means possible to virtually eliminate false-positives.

Central to the concept of nucleic acid detection is the selective amplification or purification of the desired cancer-specific markers away from the same or closely similar markers from normal cells. These approaches include: (i) multiple primer binding regions for orthogonal amplification and detection, (ii) affinity selection of CTC's or exosomes, and (iii) spatial dilution of the sample.

The success of PCR-LDR, which uses 4 primer-binding regions to assure sensitivity and specificity, has previously been demonstrated. Desired regions are amplified using pairs or even tandem pairs of PCR primers, followed by orthogonal nested LDR primer pairs for detection. One advantage of using PCR-LDR is the ability to perform proportional PCR amplification of multiple fragments to enrich for low copy targets, and then use quantitative LDR to directly identify cancer-specific mutations. Biofire/bioMerieux has developed a similar technology termed “film array”; wherein initial multiplexed PCR reaction products are redistributed into individual wells, and then nested real-time PCR performed with SYBR Green Dye detection.

Affinity purification of CTC's using antibody or aptamer capture has been demonstrated (Adams et al., J. Am. Chem. Soc. 130: 8633-8641 (2008); Dharmasiri et al., Electrophoresis 30:3289-3300 (2009); Soper et al. Biosens. Bioelectron. 21:1932-1942 (2006)). Peptide affinity capture of exosomes has been reported in the literature. Enrichment of these tumor-specific fractions from the blood enables copy number quantification, as well as simplifying screening and verification assays.

The last approach, spatial dilution of the sample, is employed in digital PCR as well as its close cousin known as BEAMing (Vogelstein and Kinzler, Proc. Natl. Acad. Sci. USA. 96(16):9236-41 (1999); Dressman et al., Proc. Natl. Acad. Sci. USA 100:8817-8822 (2003)). The rational for digital PCR is to overcome the limit of enzymatic discrimination when the sample comprises very few target molecules containing a known mutation in a 1,000 to 10,000-fold excess of wild-type DNA. By diluting input DNA into 20,000 or more droplets or beads to distribute less than one molecule of target per droplet, the DNA may be amplified via PCR, and then detected via probe hybridization or TaqMan™ reaction, giving in essence a 0/1 digital score. The approach is currently the most sensitive for finding point mutations in plasma, but it does require prior knowledge of the mutations being scored, as well as a separate digital dilution for each mutation, which would deplete the entire sample to score just a few mutations (Alcaide et al., “A Novel Multiplex Droplet Digital PCR Assay to Identify and Quantify KRAS Mutations in Clinical Specimens,” J. Mol. Diagn. 21:28-33 (2019); Guibert et al., “Liquid Biopsy of Fine-Needle Aspiration Supernatant for Lung Cancer Genotyping,” Lung Cancer 1768:193-207 (2018); Yoshida et al., “Highly Sensitive Detection of ALK Resistance Mutations in Plasma Using Droplet Digital PCR,” BMC Cancer 18:1136 (2018)).

When developing multiplexed assays, there is a tricky balance between performing enough preliminary cycles of PCR or other amplification techniques to generate sufficient copies of each mutant or methylated region such that when diluting into uniplex qPCR, multiplex qPCR, uniplex droplet PCR or multiplexed droplet PCR there are sufficient copies to get a signal if true positive; and performing too many PCR cycles such that some markers over-amplify while others are suppressed, or relative quantification is lost.

The present application is directed at overcoming these and other deficiencies in the art.

### SUMMARY

A first aspect of the present application is directed to a method for identifying, in a sample, one or more parent nucleic acid molecules containing a target nucleotide sequence differing from nucleotide sequences of other parent nucleic acid molecules in the sample by one or more nucleotides, one or more copy numbers, one or more transcript sequences, and/or one or more methylated residues. The method involves providing a sample containing one or more parent nucleic acid molecules potentially containing the target nucleotide sequence differing from the nucleotide sequences of other parent nucleic acid molecules by one or more nucleotides, one or more copy numbers, one or more transcript sequences, and/or one or more methylated residues. One or more enzymes capable of digesting deoxyuracil (dU)-containing nucleic acid molecules are then provided. One or more primary oligonucleotide primer sets are also provided. Each primary oligonucleotide primer set comprises (a) a first primary oligonucleotide primer that comprises a nucleotide sequence that is complementary to a sequence in the parent nucleic acid molecule adjacent to the target nucleotide sequence and (b) a second primary oligonucleotide primer that comprises a nucleotide sequence that is complementary to a portion of an extension product formed from the first primary oligonucleotide primer. The sample, the one or more first primary oligonucleotide primers of the primary oligonucleotide primer sets, the one or more enzymes capable of digesting deoxyuracil (dU)-containing nucleic acid molecules, a deoxynucleotide mix, and a DNA polymerase are blended to form one or more polymerase extension reaction mixtures, and the one or more polymerase extension reaction mixtures are subjected to conditions suitable for digesting deoxyuracil (dU)-containing nucleic acid molecules present in the polymerase extension reaction mixtures and for carrying out one or more polymerase extension reaction cycles comprising a denaturation treatment, a hybridization treatment, and an extension treatment, thereby forming primary extension products comprising nucleotide sequences complementary to the target nucleotide sequence. The one or more polymerase extension reaction mixtures comprising the primary extension products, the one or more second primary oligonucleotide primers of the primary oligonucleotide primer sets, the one or more enzymes capable of digesting deoxyuracil (dU)-containing nucleic acid molecules in the reaction mixtures, a deoxynucleotide mix including dUTP, and a DNA polymerase are blended to form one or more first polymerase chain reaction mixtures. The method further comprises subjecting the one or more first polymerase chain reaction mixtures to conditions suitable for digesting deoxyuracil (dU)-containing nucleic acid molecules present in the polymerase chain reaction mixtures and for carrying out one or more polymerase chain reaction cycles comprising a denaturation treatment, a hybridization treatment, and an extension treatment, thereby forming one or more first polymerase chain reaction products comprising the target nucleotide sequence or a complement thereof. One or more oligonucleotide probe sets are then provided. Each probe set comprises (a) a first oligonucleotide probe having a 5′ primer-specific portion and a 3′ target sequence-specific portion, and (b) a second oligonucleotide probe having a 5′ target sequence-specific portion and a 3′ primer-specific portion, wherein the first and second oligonucleotide probes of a probe set are configured to hybridize, in a base specific manner, on a complementary target nucleotide sequence of a secondary extension product. The one or more first polymerase chain reaction products are blended with a ligase, and the one or more oligonucleotide probe sets to form one or more ligation reaction mixtures. The one or more ligation reaction mixtures are subjected to one or more ligation reaction cycles whereby the first and second oligonucleotide probes of the one or more oligonucleotide probe sets are ligated together, when hybridized to complementary sequences, to form ligated product sequences in the ligation reaction mixtures wherein each ligated product sequence comprises the 5′ primer-specific portion, the target-specific portions, and the 3′ primer-specific portion. The method further includes providing one or more secondary oligonucleotide primer sets. Each secondary oligonucleotide primer set comprises (a) a first secondary oligonucleotide primer comprising the same nucleotide sequence as the 5′ primer-specific portion of the ligated product sequence and (b) a second secondary oligonucleotide primer comprising a nucleotide sequence that is complementary to the 3′ primer-specific portion of the ligated product sequence. The ligated product sequences, the one or more secondary oligonucleotide primer sets, the one or more enzymes capable of digesting deoxyuracil (dU)-containing nucleic acid molecules, a deoxynucleotide mix including dUTP, and a DNA polymerase are blended to form one or more second polymerase chain reaction mixtures. The one or more second polymerase chain reaction mixtures are subjected to conditions suitable for digesting deoxyuracil (dU)-containing nucleic acid molecules present in the second polymerase chain reaction mixtures and for carrying out one or more polymerase chain reaction cycles comprising a denaturation treatment, a hybridization treatment, and an extension treatment thereby forming one or more second polymerase chain reaction products. The method further comprises detecting and distinguishing the one or more second polymerase chain reaction products in the one or more second polymerase chain reaction mixtures to identify the presence of one or more parent nucleic acid molecules containing target nucleotide sequences differing from nucleotide sequences in other parent nucleic acid molecules in the sample by one or more nucleotides, one or more copy numbers, one or more transcript sequences, and/or one or more methylated residues.

Another aspect of the present application is directed to a method for identifying, in a sample, one or more parent nucleic acid molecules containing a target nucleotide sequence differing from nucleotide sequences in other parent nucleic acid molecules in the sample by one or more nucleotides, one or more copy numbers, one or more transcript sequences, and/or one or more methylated residues. The method involves providing a sample containing one or more parent nucleic acid molecules potentially containing the target nucleotide sequence differing from the nucleotide sequences of other parent nucleic acid molecules by one or more nucleotides, one or more copy numbers, one or more transcript sequences, and/or one or more methylated residues. One or more enzymes capable of digesting deoxyuracil (dU) containing nucleic acid molecules, one or more nucleases capable of digesting nucleic acid molecules not comprising modified nucleotides, and one or more first primary oligonucleotide primer(s) are provided. The one or more first primary oligonucleotide primer(s) comprise a nucleotide sequence that is complementary to a sequence in the parent nucleic acid molecule adjacent to the target nucleotide sequence. The sample, the one or more first primary oligonucleotide primers, the one or more enzymes capable of digesting deoxyuracil (dU)-containing nucleic acid molecules, a deoxynucleotide mix that comprises one or more modified nucleotides that protect extension products but not target DNA from nuclease digestion, and a DNA polymerase are blended to form one or more polymerase extension reaction mixtures, and the one or more polymerase extension reaction mixtures are subjected to conditions suitable for digesting deoxyuracil (dU)-containing nucleic acid molecules present in the polymerase extension reaction mixture and for carrying out one or more polymerase extension reaction cycles comprising a denaturation treatment, a hybridization treatment, and an extension treatment, thereby forming primary extension products comprising the complement of the target nucleotide sequence. The method further comprises providing one or more secondary oligonucleotide primer sets. Each secondary oligonucleotide primer set comprises (a) a first secondary oligonucleotide primer having a first 5′ primer-specific portion and a 3′ portion that is complementary to a portion of a primary extension product formed from the first primary oligonucleotide primer and (b) a second secondary oligonucleotide primer having a second 5′ primer-specific portion and a 3′ portion that comprises a nucleotide sequence that is complementary to a portion of an extension product formed from the first secondary oligonucleotide primer. The one or more polymerase extension reaction mixtures comprising the primary extension products, the one or more secondary oligonucleotide primer sets, the one or more nucleases, a deoxynucleotide mix, and a DNA polymerase are blended to form one or more first polymerase chain reaction mixtures, and the one or more first polymerase chain reaction mixtures are subjected to conditions suitable for digesting nucleic acid molecules present in the first polymerase chain reaction mixtures, but not primary extension products comprising modified nucleotides and for carrying out two or more polymerase chain reaction cycles comprising a denaturation treatment, a hybridization treatment, and an extension treatment, thereby forming one or more first polymerase chain reaction products comprising the first 5′ primer-specific portion, a target-specific nucleotide sequence or a complement thereof, and a complement of the second 5′ primer-specific portion. One or more tertiary oligonucleotide primer sets are provided. Each tertiary oligonucleotide primer set comprises (a) a first tertiary oligonucleotide primer comprising the same nucleotide sequence as the first 5′ primer-specific portion of the one or more first polymerase chain reaction products and (b) a second tertiary oligonucleotide primer comprising a nucleotide sequence that is complementary to the 3′ primer-specific portion of the one or more first polymerase chain reaction products. The one or more first polymerase chain reaction products, the one or more tertiary oligonucleotide primer sets, the one or more enzymes capable of digesting deoxyuracil (dU) containing nucleic acid molecules, a deoxynucleotide mix including dUTP, and a DNA polymerase are blended to form one or more second polymerase chain reaction mixtures, and the one or more second polymerase chain reaction mixtures are subjected to conditions suitable for digesting deoxyuracil (dU) containing nucleic acid molecules present in the second polymerase chain reaction mixtures and for carrying out one or more polymerase chain reaction cycles comprising a denaturation treatment, a hybridization treatment, and an extension treatment, thereby forming one or more second polymerase chain reaction products. The method further involves detecting and distinguishing the one or more second polymerase chain reaction products in the one or more second polymerase chain reaction mixtures to identify the presence of one or more parent nucleic acid molecules containing target nucleotide sequences differing from nucleotide sequences in other parent nucleic acid molecules in the sample by one or more nucleotides, one or more copy numbers, one or more transcript sequences, and/or one or more methylated residues.

Another aspect of the present application is directed to a method for identifying, in a sample, one or more parent nucleic acid molecules containing a target nucleotide sequence differing from nucleotide sequences of other parent nucleic acid molecules in the sample by one or more nucleotides, one or more copy numbers, one or more transcript sequences, and/or one or more methylated residues. The method involves providing a sample containing one or more parent nucleic acid molecules potentially containing the target nucleotide sequence differing from the nucleotide sequences of other parent nucleic acid molecules by one or more nucleotides, one or more copy numbers, one or more transcript sequences, and/or one or more methylated residues. One or more enzymes capable of digesting deoxyuracil (dU)-containing nucleic acid molecules and one or more nucleases capable of digesting nucleic acid molecules present not comprising modified nucleotides are provided. The method also involves providing one or more primary oligonucleotide primer sets. Each primary oligonucleotide primer set comprises (a) a first primary oligonucleotide primer that comprises a nucleotide sequence that is complementary to a sequence in the parent nucleic acid molecule adjacent to the target nucleotide sequence and (b) a second primary oligonucleotide primer that comprises a nucleotide sequence that is complementary to a portion of an extension product formed from the first primary oligonucleotide primer. The sample, the one or more first primary oligonucleotide primers of the primary oligonucleotide primer sets, the one or more enzymes capable of digesting deoxyuracil (dU)-containing nucleic acid molecules, a deoxynucleotide mix that comprises one or more modified nucleotides that protect extension product but not target DNA from nuclease digestion, and a DNA polymerase are blended to form one or more polymerase extension reaction mixtures, and the one or more polymerase extension reaction mixtures are subjected to conditions suitable for digesting deoxyuracil (dU)-containing nucleic acid molecules present in the polymerase extension reaction mixtures and for carrying out one or more polymerase extension reaction cycles comprising a denaturation treatment, a hybridization treatment, and an extension treatment, thereby forming primary extension products comprising the complement of the target nucleotide sequence. The method further comprises blending the one or more polymerase extension reaction mixtures comprising the primary extension products, the one or more second primary oligonucleotide primers of the one or more primary oligonucleotide primer sets, the one or more nucleases, a deoxynucleotide mix, and a DNA polymerase to form one or more first polymerase chain reaction mixtures. The one or more first polymerase chain reaction mixtures are subjected to conditions suitable for digesting nucleic acid molecules present in the polymerase chain reaction mixtures, but not primary extension products comprising modified nucleotides and for carrying out two or more polymerase chain reaction cycles comprising a denaturation treatment, a hybridization treatment, and an extension treatment, thereby forming first polymerase chain reaction products comprising the target nucleotide sequence or a complement thereof. One or more secondary oligonucleotide primer sets are then provided. Each secondary oligonucleotide primer set comprises (a) a first secondary oligonucleotide primer having a 3′ portion that is complementary to a portion of an extension product formed from the first primary oligonucleotide primer and (b) a second secondary oligonucleotide primer having a 3′ portion that comprises a nucleotide sequence that is complementary to a portion of an extension product formed from the first secondary oligonucleotide primer. The first polymerase chain reaction products, the one or more secondary oligonucleotide primer sets, the one or more enzymes capable of digesting deoxyuracil (dU) containing nucleic acid molecules, a deoxynucleotide mix including dUTP, and a DNA polymerase are blended to form one or more second polymerase chain reaction mixtures. The one or more second polymerase chain reaction mixtures are subjected to conditions suitable for digesting deoxyuracil (dU)-containing nucleic acid molecules present in the second polymerase chain reaction mixtures and for carrying out two or more polymerase chain reaction cycles comprising a denaturation treatment, a hybridization treatment, and an extension treatment thereby forming second polymerase chain reaction products. The method further comprises detecting and distinguishing the second polymerase chain reaction products in the one or more second polymerase chain reaction mixtures to identify the presence of one or more parent nucleic acid molecules containing target nucleotide sequences differing from nucleotide sequences in other parent nucleic acid molecules in the sample by one or more nucleotides, one or more copy numbers, one or more transcript sequences, and/or one or more methylated residues.

Another aspect of the present application is directed to a method for identifying, in a sample, one or more parent nucleic acid molecules containing a target nucleotide sequence differing from nucleotide sequences in other parent nucleic acid molecules in the sample by one or more methylated residues. The method involves providing a sample containing one or more parent nucleic acid molecules potentially containing the target nucleotide sequence differing from the nucleotide sequences in other parent nucleic acid molecules by one or more methylated residues and subjecting the nucleic acid molecules in the sample to a bisulfite treatment under conditions suitable to convert unmethylated cytosine residues to uracil residues. One or more enzymes capable of digesting deoxyuracil (dU)-containing nucleic acid molecules are then provided. The method further involves providing one or more primary oligonucleotide primer sets. Each primary oligonucleotide primer set comprises (a) a first primary oligonucleotide primer that comprises a nucleotide sequence that is complementary to a sequence in the bisulfite-treated parent nucleic acid molecules adjacent to the bisulfite-treated target nucleotide sequence containing the one or more methylated residue and (b) a second primary oligonucleotide primer that comprises a nucleotide sequence that is complementary to a portion of an extension product formed from the first primary oligonucleotide primer. The bisulfate-treated sample, the one or more first primary oligonucleotide primers of the one or more primary oligonucleotide primer sets, a deoxynucleotide mix, and a DNA polymerase are blended to form one or more polymerase extension reaction mixtures, and the one or more polymerase extension reaction mixtures are subjected to conditions suitable for one or more polymerase extension reaction cycles comprising a denaturation treatment, a hybridization treatment, and an extension treatment, thereby forming primary extension products comprising the complement of the bisulfite-treated target nucleotide sequence. The one or more polymerase extension reaction mixtures comprising the primary extension products, the one or more secondary primary oligonucleotide primers of the one or more primary oligonucleotide primer sets, the one or more enzymes capable of digesting deoxyuracil (dU)-containing nucleic acid molecules, a deoxynucleotide mix including dUTP, and a DNA polymerase are blended to form one or more first polymerase chain reaction mixtures. The one or more first polymerase chain reaction mixtures are subjected to conditions suitable for digesting deoxyuracil (dU)-containing nucleic acid molecules present in the first polymerase chain reaction mixtures and for carrying out one or more polymerase chain reaction cycles comprising a denaturation treatment, a hybridization treatment, and an extension treatment, thereby forming first polymerase chain reaction products comprising the bisulfite-treated target nucleotide sequence or a complement thereof. The method further involves providing one or more oligonucleotide probe sets. Each probe set comprises (a) a first oligonucleotide probe having a 5′ primer-specific portion and a 3′ bisulfite-treated target nucleotide sequence-specific or complement sequence-specific portion, and (b) a second oligonucleotide probe having a 5′ bisulfite-treated target nucleotide sequence-specific or complement sequence-specific portion and a 3′ primer-specific portion, and wherein the first and second oligonucleotide probes of a probe set are configured to hybridize, in a base specific manner, on a complementary nucleotide sequence of a first polymerase chain reaction product. The first polymerase chain reaction products are blended with a ligase and the one or more oligonucleotide probe sets to form one or more ligation reaction mixtures. The one or more ligation reaction mixtures are subjected to one or more ligation reaction cycles whereby the first and second oligonucleotide probes of the one or more oligonucleotide probe sets are ligated together, when hybridized to complementary sequences, to form ligated product sequences in the ligation reaction mixture wherein each ligated product sequence comprises the 5′ primer-specific portion, the bisulfite-treated target nucleotide sequence-specific or complement sequence-specific portions, and the 3′ primer-specific portion. The method further comprises providing one or more secondary oligonucleotide primer sets. Each secondary oligonucleotide primer set comprises (a) a first secondary oligonucleotide primer comprising the same nucleotide sequence as the 5′ primer-specific portion of the ligated product sequence and (b) a second secondary oligonucleotide primer comprising a nucleotide sequence that is complementary to the 3′ primer-specific portion of the ligated product sequence. The ligated product sequences, the one or more secondary oligonucleotide primer sets, the one or more enzymes capable of digesting deoxyuracil (dU)-containing nucleic acid molecules, a deoxynucleotide mix including dUTP, and a DNA polymerase are blended to form one or more second polymerase chain reaction mixtures, and the one or more second polymerase chain reaction mixtures are subjected to conditions suitable for digesting deoxyuracil (dU)-containing nucleic acid molecules present in the second polymerase chain reaction mixtures and for carrying out one or more polymerase chain reaction cycles comprising a denaturation treatment, a hybridization treatment, and an extension treatment thereby forming a second polymerase chain reaction products. The method further involves detecting and distinguishing the second polymerase chain reaction products in the one or more second polymerase chain reaction mixtures to identify the presence of one or more nucleic acid molecules containing target nucleotide sequences differing from nucleotide sequences in other parent nucleic acid molecules in the sample by one or more methylated residues.

Another aspect of the present application is directed to a method for identifying, in a sample, one or more parent nucleic acid molecules containing a target nucleotide sequence differing from nucleotide sequences in other parent nucleic acid molecules in the sample by one or more methylated residues. The method involves providing a sample containing one or more parent nucleic acid molecules potentially containing the target nucleotide sequence differing from the nucleotide sequences in other parent nucleic acid molecules by one or more methylated residues. The nucleic acid molecules in the sample are subjected to a bisulfite treatment under conditions suitable to convert unmethylated cytosine residues to uracil residues. One or more enzymes capable of digesting deoxyuracil (dU)-containing nucleic acid molecules are provided, and one or more first primary oligonucleotide primer(s) are provided. Each first primary oligonucleotide primer comprises a nucleotide sequence that is complementary to a sequence in the bisulfite-treated parent nucleic acid molecules adjacent to the bisulfite-treated target nucleotide sequence containing the one or more methylated residue. The bisulfite-treated sample, the one or more first primary oligonucleotide primers, a deoxynucleotide mix, and a DNA polymerase are blended to form one or more polymerase extension reaction mixtures, and the one or more polymerase extension reaction mixtures are subjected to conditions suitable for one or more polymerase extension reaction cycles comprising a denaturation treatment, a hybridization treatment, and an extension treatment, to form primary extension products comprising the complement of the bisulfite-treated target nucleotide sequence. The method further comprises providing one or more secondary oligonucleotide primer sets. Each secondary oligonucleotide primer set comprises (a) a first secondary oligonucleotide primer having a 5′ primer-specific portion and a 3′ portion that is complementary to a portion of the polymerase extension reaction product formed from the first primary oligonucleotide primer and (b) a second secondary oligonucleotide primer having a 5′ primer-specific portion and a 3′ portion that comprises a nucleotide sequence that is complementary to a portion of an extension product formed from the first secondary oligonucleotide primer. The one or more polymerase extension reaction mixtures comprising the primary extension products, the one or more secondary oligonucleotide primer sets, the one or more enzymes capable of digesting deoxyuracil (dU)-containing nucleic acid molecules, a deoxynucleotide mix, and a DNA polymerase are blended to form one or more first polymerase chain reaction mixtures, and the one or more first polymerase chain reaction mixtures are subjected to conditions suitable for digesting nucleic acid molecules present in the first polymerase chain reaction mixtures, but not primary extension products comprising modified nucleotides and for carrying out two or more polymerase chain reaction cycles comprising a denaturation treatment, a hybridization treatment, and an extension treatment, thereby forming first polymerase chain reactions products comprising a 5′ primer-specific portion of the first secondary oligonucleotide primer, the bisulfite-treated target nucleotide sequence-specific or complement sequence-specific portion, and a complement of the 5′ primer-specific portion of the second secondary oligonucleotide primer. The method further involves providing one or more tertiary oligonucleotide primer sets. Each tertiary oligonucleotide primer set comprises (a) a first tertiary oligonucleotide primer comprising the same nucleotide sequence as the 5′ primer-specific portion of the first polymerase chain reaction products and (b) a second tertiary oligonucleotide primer comprising a nucleotide sequence that is complementary to the 3′ primer-specific portion of the first polymerase chain reactions product sequence. The first polymerase chain reaction products, the one or more tertiary oligonucleotide primer sets, the one or more enzymes capable of digesting deoxyuracil (dU) containing nucleic acid molecules, a deoxynucleotide mix including dUTP, and a DNA polymerase are blended to form one or more second polymerase chain reaction mixtures. The one or more second polymerase chain reaction mixtures are subjected to conditions suitable for digesting deoxyuracil (dU)-containing nucleic acid molecules present in the second polymerase chain reaction mixtures and for carrying out one or more polymerase chain reaction cycles comprising a denaturation treatment, a hybridization treatment, and an extension treatment thereby forming secondary polymerase chain reaction products. The method further involves detecting and distinguishing the secondary polymerase chain reactions products in the one or more second polymerase chain reaction mixtures to identify the presence of one or more parent nucleic acid molecules containing target nucleotide sequences differing from nucleotide sequences in other parent nucleic acid molecules in the sample by one or more methylated residues.

Another aspect of the present application is directed to a method for identifying, in a sample, one or more parent nucleic acid molecules containing a target nucleotide sequence differing from nucleotide sequences in other parent nucleic acid molecules in the sample by one or more methylated residues. The method involves providing a sample containing one or more parent nucleic acid molecules potentially containing the target nucleotide sequence differing from the nucleotide sequences in other parent nucleic acid molecules by one or more methylated residues. The nucleic acid molecules in the sample are subjected to a bisulfite treatment under conditions suitable to convert unmethylated cytosine residues to uracil residues. One or more enzymes capable of digesting deoxyuracil (dU)-containing nucleic acid molecules present in the sample are provided, and one or more primary oligonucleotide primer sets are provided. Each primary oligonucleotide primer set comprises (a) a first primary oligonucleotide primer that comprises a nucleotide sequence that is complementary to a sequence in the bisulfite-treated parent nucleic acid molecules adjacent to the bisulfite-treated target nucleotide sequence containing the one or more methylated residue and (b) a second primary oligonucleotide primer that comprises a nucleotide sequence that is complementary to a portion of an extension product formed from the first primary oligonucleotide primer. The bisulfite-treated sample, the one or more first primary oligonucleotide primers of the one or more primary oligonucleotide primer sets, a deoxynucleotide mix, and a DNA polymerase are blended to form one or more polymerase extension reaction mixtures. The one or more polymerase extension reaction mixtures to are subjected to conditions suitable for one or more polymerase extension reaction cycles comprising a denaturation treatment, a hybridization treatment, and an extension treatment, thereby forming primary extension products comprising the complement of the bisulfite treated target nucleotide sequence. The one or more polymerase extension reaction mixtures comprising the primary extension products, the one or more secondary primary oligonucleotide primers of the one or more primary oligonucleotide primer sets, the one or more enzymes capable of digesting deoxyuracil (dU)-containing nucleic acid molecules in the reaction mixture, a deoxynucleotide mix, and a DNA polymerase are blended to form one or more first polymerase chain reaction mixtures. The one or more first polymerase chain reaction mixtures are subjected to conditions suitable for digesting deoxyuracil (dU)-containing nucleic acid molecules present in the first polymerase chain reaction mixtures and for carrying out one or more polymerase chain reaction cycles comprising a denaturation treatment, a hybridization treatment, and an extension treatment, thereby forming first polymerase chain reaction products comprising the bisulfite-treated target nucleotide sequence or a complement thereof. The method further comprises providing one or more secondary oligonucleotide primer sets. Each secondary oligonucleotide primer set comprises (a) a first secondary oligonucleotide primer having a 3′ portion that is complementary to a portion of a first polymerase chain reaction product formed from the first primary oligonucleotide primer and (b) a second secondary oligonucleotide primer having a 3′ portion that comprises a nucleotide sequence that is complementary to a portion of a first polymerase chain reaction product formed from the first secondary oligonucleotide primer. The first polymerase chain reaction products, the one or more secondary oligonucleotide primer sets, the one or more enzymes capable of digesting deoxyuracil (dU)-containing nucleic acid molecules, a deoxynucleotide mix including dUTP, and a DNA polymerase are blended to form one or more second polymerase chain reaction mixtures. The one or more second polymerase chain reaction mixtures are subjected to conditions suitable for digesting deoxyuracil (dU)-containing nucleic acid molecules present in the second polymerase chain reaction mixtures and for carrying out two or more polymerase chain reaction cycles comprising a denaturation treatment, a hybridization treatment, and an extension treatment thereby forming second polymerase chain reaction products. The method further involves detecting and distinguishing the second polymerase chain reactions products in the one or more second polymerase chain reaction mixtures to identify the presence of one or more parent nucleic acid molecules containing target nucleotide sequences differing from nucleotide sequences in other parent nucleic acid molecules in the sample by one or more methylated residues.

Another aspect of the present application is directed to a method for identifying, in a sample, one or more parent nucleic acid molecules containing a target nucleotide sequence differing from nucleotide sequences in other parent nucleic acid molecules in the sample by one or more methylated residues. The method involves providing a sample containing one or more parent nucleic acid molecules potentially containing the target nucleotide sequence differing from the nucleotide sequences in other parent nucleic acid molecules by one or more methylated residues, and subjecting the nucleic acid molecules in the sample to a bisulfite treatment under conditions suitable to convert unmethylated cytosine residues to uracil residues. One or more enzymes capable of digesting deoxyuracil (dU)-containing nucleic acid molecules present in the sample are provided. One or more primary oligonucleotide primer sets are also provided. Each primary oligonucleotide primer set comprises (a) a first primary oligonucleotide primer having a 5′ primer-specific portion and a 3′ portion that comprises a nucleotide sequence that is complementary to a sequence in the bisulfite-treated parent nucleic acid molecules adjacent to the bisulfite-treated target nucleotide sequence containing the one or more methylated residue and (b) a second primary oligonucleotide primer having a 5′ primer-specific portion and a 3′ portion that comprises a nucleotide sequence that is complementary to a portion of an extension product formed from the first primary oligonucleotide primer. The bisulfate treated sample, the one or more first primary oligonucleotide primers of the one or more primary oligonucleotide primer sets, a deoxynucleotide mix, and a DNA polymerase are blended to form one or more polymerase extension reaction mixtures. The one or more polymerase extension reaction mixtures are subjected to conditions suitable for one or more polymerase extension reaction cycles comprising a denaturation treatment, a hybridization treatment, and an extension treatment, thereby forming primary extension products comprising the complement of the bisulfate treated target nucleotide sequence. The one or more polymerase extension reaction mixtures comprising the primary extension products, the one or more secondary primary oligonucleotide primers of the one or more primary oligonucleotide primer sets, the one or more enzymes capable of digesting deoxyuracil (dU)-containing nucleic acid molecules in the reaction mixture, a deoxynucleotide mix, and a DNA polymerase are blended to form one or more first polymerase chain reaction mixtures. The method further comprises subjecting the one or more first polymerase chain reaction mixtures to conditions suitable for digesting deoxyuracil (dU)-containing nucleic acid molecules present in the polymerase chain reaction mixtures and for carrying out one or more polymerase chain reaction cycles comprising a denaturation treatment, a hybridization treatment, and an extension treatment, thereby forming first polymerase chain reactions products comprising the bisulfite-treated target nucleotide sequence or a complement thereof. One or more secondary oligonucleotide primer sets are provided. Each secondary oligonucleotide primer set comprises (a) a first secondary oligonucleotide primer comprising the same nucleotide sequence as the 5′ primer-specific portion of the first polymerase chain reaction products or their complements and (b) a second secondary oligonucleotide primer comprising a nucleotide sequence that is complementary to the 3′ primer-specific portion of the first polymerase chain reaction products or their complements. The primary polymerase chain reaction product sequences, the one or more secondary oligonucleotide primer sets, the one or more enzymes capable of digesting deoxyuracil (dU)-containing nucleic acid molecules, a deoxynucleotide mix including dUTP, and a DNA polymerase are blended to form one or more second polymerase chain reaction mixtures. The one or more second polymerase chain reaction mixtures are subjected to conditions suitable for digesting deoxyuracil (dU)-containing nucleic acid molecules present in the second polymerase chain reaction mixtures and for carrying out one or more polymerase chain reaction cycles comprising a denaturation treatment, a hybridization treatment, and an extension treatment thereby forming second polymerase chain reaction products. The method further involves detecting and distinguishing the second polymerase chain reactions products in the one or more second polymerase chain reaction mixtures to identify the presence of one or more parent nucleic acid molecules containing target nucleotide sequences differing from nucleotide sequences in other parent nucleic acid molecules in the sample by one or more methylated residues.

Another aspect of the present application is directed to a method for identifying in a sample, one or more parent ribonucleic acid molecules containing a target ribonucleic acid sequence differing from ribonucleic acid sequences of other parent ribonucleic acid molecules in the sample due to alternative splicing, alternative transcript, alternative start site, alternative coding sequence, alternative non-coding sequence, exon insertion, exon deletion, intron insertion, translocation, mutation, or other rearrangement at the genome level. The method involves providing a sample containing one or more parent ribonucleic acid molecules containing a target ribonucleic acid molecule potentially differing in sequence from other parent ribonucleic acid molecules and providing one or more enzymes capable of digesting deoxyuracil (dU) containing nucleic acid molecules present in the sample. The sample is contacted with one or more enzymes capable of digesting dU containing nucleic acid molecules potentially present in the sample. One or more primary oligonucleotide primer sets are then provided. Each primary oligonucleotide primer set comprises (a) a first primary oligonucleotide primer that comprises a nucleotide sequence that is complementary to the RNA sequence in the parent ribonucleic acid molecule adjacent to the target ribonucleotide sequence and (b) a second primary oligonucleotide primer that comprises a nucleotide sequence that is complementary to a portion of the cDNA extension product formed from the first primary oligonucleotide primer. The contacted sample, the one or more primary oligonucleotide primer sets, a deoxynucleotide mix including dUTP, a reverse transcriptase, and a DNA polymerase or a DNA polymerase with reverse-transcriptase activity are blended to form one or more reverse-transcription/polymerase chain reaction mixtures. The one or more reverse-transcription/polymerase chain reaction mixtures are subjected to conditions suitable for generating complementary deoxyribonucleic acid (cDNA) molecules to the target ribonucleic nucleic acid and to carry out one or more polymerase chain reaction cycles comprising a denaturation treatment, a hybridization treatment, and an extension treatment thereby forming one or more different reverse transcription/polymerase products. The method further comprises providing one or more oligonucleotide probe sets. Each probe set comprises (a) a first oligonucleotide probe having a 5′ primer-specific portion and a 3′ target sequence-specific portion, and (b) a second oligonucleotide probe having a 5′ target sequence-specific portion and a 3′ primer-specific portion, wherein the first and second oligonucleotide probes of a probe set are configured to hybridize, in a base specific manner, on complementary portions of a reverse transcriptase/polymerase product corresponding to the target ribonucleic acid molecule sequence. The reverse transcriptase/polymerase products are contacted with a ligase and the one or more oligonucleotide probe sets to form one or more ligation reaction mixtures, and the one or more ligation reaction mixtures are subjected to one or more ligation reaction cycles whereby the first and second probes of the one or more oligonucleotide probe sets, when hybridized to their complement, are ligated together to form ligated product sequences in the ligase reaction mixture, wherein each ligated product sequence comprises the 5′ primer-specific portion, the target-specific portions, and the 3′ primer-specific portion. The method further involves providing one or more secondary oligonucleotide primer sets. Each secondary oligonucleotide primer set comprises (a) a first secondary oligonucleotide primer comprising the same nucleotide sequence as the 5′ primer-specific portion of the ligated product sequence and (b) a second secondary oligonucleotide primer comprising a nucleotide sequence that is complementary to the 3′ primer-specific portion of the ligated product sequence. The ligated product sequences, the one or more secondary oligonucleotide primer sets with one or more enzymes capable of digesting deoxyuracil (dU)-containing nucleic acid molecules, a deoxynucleotide mix including dUTP, and a DNA polymerase are blended to form one or more first polymerase chain reaction mixtures, and the one or more first polymerase chain reaction mixtures are subjected to conditions suitable for digesting deoxyuracil (dU)-containing nucleic acid molecules present in the first polymerase chain reaction mixtures and for carrying out one or more polymerase chain reaction cycles comprising a denaturation treatment, a hybridization treatment, and an extension treatment thereby forming first polymerase chain reaction products. The method further comprises detecting and distinguishing the first polymerase chain reaction products, thereby identifying the presence of one or more parent ribonucleic acid molecules containing a target ribonucleic acid sequence differing from ribonucleic acid sequences of other parent ribonucleic acid molecules in the sample due to alternative splicing, alternative transcript, alternative start site, alternative coding sequence, alternative non-coding sequence, exon insertion, exon deletion, intron insertion, translocation, mutation, or other rearrangement at the genome level.

Another aspect of the present application is directed to a method for identifying in a sample, one or more parent ribonucleic acid molecules containing a target ribonucleic acid sequence differing from ribonucleic acid sequences of other parent ribonucleic acid molecules in the sample due to alternative splicing, alternative transcript, alternative start site, alternative coding sequence, alternative non-coding sequence, exon insertion, exon deletion, intron insertion, translocation, mutation, or other rearrangement at the genome level. The method involves providing a sample containing one or more parent ribonucleic acid molecules containing a target ribonucleic acid molecule potentially differing in sequence from other parent ribonucleic acid molecules and providing one or more enzymes capable of digesting deoxyuracil (dU)-containing nucleic acid molecules present in the sample. The sample is contacted with one or more enzymes capable of digesting dU containing nucleic acid molecules potentially present in the sample. The method further involves providing one or more primary oligonucleotide primer sets, each primary oligonucleotide primer set comprising (a) a first primary oligonucleotide primer that comprises a nucleotide sequence that is complementary to the RNA sequence in the parent ribonucleic acid molecule adjacent to the target nucleotide sequence and (b) a second primary oligonucleotide primer that comprises a nucleotide sequence that is complementary to a portion of the cDNA extension product formed from the first primary oligonucleotide primer. The contacted sample, the one or more primary oligonucleotide primer sets, a deoxynucleotide mix, a reverse transcriptase and a DNA polymerase or a DNA polymerase with reverse-transcriptase activity are blended to form one or more reverse-transcription/polymerase chain reaction mixtures, and the one or more reverse-transcription/polymerase chain reaction mixtures are subjected to conditions suitable for generating complementary deoxyribonucleic acid (cDNA) molecules to the target RNA and to carry out one or more polymerase chain reaction cycles comprising a denaturation treatment, a hybridization treatment, and an extension treatment thereby forming one or more different reverse-transcription/primary polymerase chain reaction products. The method further comprises providing one or more secondary oligonucleotide primer sets. Each secondary oligonucleotide primer set comprises (a) a first secondary oligonucleotide primer having a 3′ portion that is complementary to a portion of a reverse-transcription/primary polymerase chain reaction product formed from the first primary oligonucleotide primer and (b) a second secondary oligonucleotide primer having a 3′ portion that comprises a nucleotide sequence that is complementary to a portion of a reverse-transcription/primary polymerase chain reaction product formed from the first secondary oligonucleotide primer. The reverse-transcription/primary polymerase chain reaction products, the one or more secondary oligonucleotide primer sets, the one or more enzymes capable of digesting deoxyuracil (dU) containing nucleic acid molecules, a deoxynucleotide mix including dUTP, and a DNA polymerase are blended to form one or more first polymerase chain reaction mixtures, and the one or more first polymerase chain reaction mixtures are subjected to conditions suitable for digesting deoxyuracil (dU)-containing nucleic acid molecules present in the first polymerase chain reaction mixtures and for carrying out two or more polymerase chain reaction cycles comprising a denaturation treatment, a hybridization treatment, and an extension treatment thereby forming first polymerase chain reaction products. The method further involves detecting and distinguishing the first polymerase chain reaction products, thereby identifying the presence of one or more parent ribonucleic acid molecules containing a target ribonucleic acid sequences differing from ribonucleic acid sequences of other parent ribonucleic acid molecules in the sample due to alternative splicing, alternative transcript, alternative start site, alternative coding sequence, alternative non-coding sequence, exon insertion, exon deletion, intron insertion, translocation, mutation, or other rearrangement at the genome level.

Another aspect of the present application is directed to a method for identifying, in a sample, one or more target micro-ribonucleic acid (miRNA) molecules differing in sequence from other miRNA molecules in the sample by one or more bases. The method involves providing a sample containing one or more target miRNA molecules potentially differing in sequence from other miRNA molecules in the sample by one or more bases and providing one or more enzymes capable of digesting deoxyuracil (dU)-containing nucleic acid molecules present in the sample. The sample is contacted with one or more enzymes capable of digesting dU-containing nucleic acid molecules potentially present in the sample, and the contacted sample is blended with a ligase and one or more first oligonucleotide preliminary probes comprising a 5′ phosphate, a 5′ stem-loop portion, an internal primer-specific portion within the loop region, a blocking group, and a 3′ nucleotide sequence that is complementary to a 3′ portion of the target miRNA molecule sequence to form one or more first ligation reaction mixtures. The method further comprises ligating, in the one or more first ligation reaction mixtures, the one or more target miRNA molecules at their 3′ end to the 5′ phosphate of the one or more first oligonucleotide preliminary probes to generate chimeric nucleic acid molecules comprising the target miRNA molecule sequence, if present in the sample, appended to the one or more first oligonucleotide preliminary probes. One or more primary oligonucleotide primer sets are then provided. Each primer set comprises (a) a first primary oligonucleotide primer comprising a nucleotide sequence that is complementary to the internal primer-specific portion of the first oligonucleotide preliminary probe, and (b) a second primary oligonucleotide primer comprising a 5′ primer-specific portion and a 3′ portion, wherein the second primary oligonucleotide primer may be the same or may differ from other second primary oligonucleotide primers in other sets. The one or more first ligation reaction mixtures comprising chimeric nucleic acid molecules, the one or more primary oligonucleotide primer sets, the one or more enzymes capable of digesting deoxyuracil (dU)-containing nucleic acid molecules in the sample, a deoxynucleotide mix including dUTP, and a reverse transcriptase and a DNA polymerase or a DNA polymerase with reverse-transcriptase activity are blended to form one or more reverse-transcription/polymerase chain reaction mixtures. The one or more reverse-transcription/polymerase chain reaction mixtures are subjected to conditions suitable for digesting deoxyuracil (dU)-containing nucleic acid molecules present in the reverse-transcription/polymerase chain reaction mixtures to conditions suitable for generating complementary deoxyribonucleic acid (cDNA) molecules to the chimeric nucleic acid molecules, and to one or more polymerase chain reaction cycles comprising a denaturation treatment, a hybridization treatment, and an extension treatment thereby forming one or more different primary reverse-transcription/polymerase chain reaction products comprising the 5′ primer-specific portion, a nucleotide sequence corresponding to the target miRNA molecule sequence, and the complement of the internal primer-specific portion, and complements thereof. The method further involves providing one or more oligonucleotide probe sets. Each probe set comprises (a) a first oligonucleotide probe having a 5′ primer-specific portion and a 3′ target sequence-specific portion, and (b) a second oligonucleotide probe having a 5′ target sequence-specific portion, a portion complementary to a primary extension product, and a 3′ primer-specific portion, wherein the first and second oligonucleotide probes of a probe set are configured to hybridize, in a base specific manner, on complementary portions of a primary reverse-transcription/polymerase chain reaction product corresponding to the target miRNA molecule sequence, or complement thereof. The primary reverse-transcription/polymerase chain reaction products are contacted with a ligase and the one or more oligonucleotide probe sets to form one or more second ligation reaction mixtures, and the one or more second ligation reaction mixtures are subjected to one or more ligation reaction cycles whereby the first and second oligonucleotide probes of the one or more oligonucleotide probe sets, when hybridized to their complement, are ligated together to form ligated product sequences in the ligation reaction mixture, wherein each ligated product sequence comprises the 5′ primer-specific portion, the target-specific portions, and the 3′ primer-specific portion. The method further involves providing one or more secondary oligonucleotide primer sets. Each secondary oligonucleotide primer set comprises (a) a first secondary oligonucleotide primer comprising the same nucleotide sequence as the 5′ primer-specific portion of the ligated product sequence and (b) a second secondary oligonucleotide primer comprising a nucleotide sequence that is complementary to the 3′ primer-specific portion of the ligated product sequence. The ligated product sequences and the one or more secondary oligonucleotide primer sets are blended with one or more enzymes capable of digesting deoxyuracil (dU)-containing nucleic acid molecules, a deoxynucleotide mix including dUTP, and a DNA polymerase to form one or more second polymerase chain reaction mixtures. The one or more second polymerase chain reaction mixtures are subjected to conditions suitable for digesting deoxyuracil (dU)-containing nucleic acid molecules present in the second polymerase chain reaction mixtures and for carrying out one or more polymerase chain reaction cycles comprising a denaturation treatment, a hybridization treatment, and an extension treatment thereby forming secondary polymerase chain reaction products. The method further comprises detecting and distinguishing the secondary polymerase chain reaction products in the one or more reactions thereby identifying one or more target miRNA molecules differing in sequence from other miRNA molecules in the sample by one or more bases.

Another aspect of the present application is directed to a method for identifying, in a sample, one or more target micro-ribonucleic acid (miRNA) molecules differing in sequence from other miRNA molecules in the sample by one or more bases. The method involves providing a sample containing one or more target miRNA molecules potentially differing in sequence from other miRNA molecules in the sample by one or more bases and providing one or more enzymes capable of digesting deoxyuracil (dU)-containing nucleic acid molecules present in the sample. The sample is contacted with one or more enzymes capable of digesting dU-containing nucleic acid molecules potentially present in the sample, and the contacted sample is blended with a ligase and one or more first oligonucleotide probes comprising a 5′ phosphate, a 5′ stem-loop portion, an internal primer-specific portion within the loop region, a blocking group, and a 3′ nucleotide sequence that is complementary to a 3′ portion of the target miRNA molecule sequence to form one or more ligation reaction mixtures. The method further involves ligating, in the one or more ligation reaction mixtures, the one or more target miRNA molecules at their 3′ end to the 5′ phosphate of the one or more first oligonucleotide probes to generate chimeric nucleic acid molecules comprising the target miRNA molecule sequence, if present in the sample, appended to the one or more first oligonucleotide probes. One or more primary oligonucleotide primer sets are then provided. Each primer set comprises (a) a first primary oligonucleotide primer comprising a nucleotide sequence that is complementary to the internal primer-specific portion of the first oligonucleotide probe, and (b) a second primary oligonucleotide primer comprising a 5′ primer-specific portion and a 3′ portion, wherein the second primary oligonucleotide primer may be the same or may differ from other second primary oligonucleotide primers in other sets. The one or more ligation reaction mixtures comprising chimeric nucleic acid molecules, the one or more primary oligonucleotide primer sets, a deoxynucleotide mix, and a reverse transcriptase and a DNA polymerase or a DNA polymerase with reverse-transcriptase activity are blended to form one or more reverse-transcription/polymerase chain reaction mixtures. The one or more reverse-transcription/polymerase chain reaction mixtures are subjected to conditions suitable for generating complementary deoxyribonucleic acid (cDNA) molecules to the chimeric nucleic acid molecules, and to one or more polymerase chain reaction cycles comprising a denaturation treatment, a hybridization treatment, and an extension treatment thereby forming one or more different primary reverse-transcription/polymerase chain reaction products comprising the 5′ primer-specific portion, a nucleotide sequence corresponding to the target miRNA molecule sequence, and the complement of the internal primer-specific portion, and complements thereof. The method further comprises providing one or more secondary oligonucleotide primer sets. Each secondary oligonucleotide primer set comprises (a) a first secondary oligonucleotide primer having a 5′ primer-specific portion and a 3′ portion that is complementary to a portion of an extension product formed from the first primary oligonucleotide primer and (b) a second secondary oligonucleotide primer having a 5′ primer-specific portion and a 3′ portion that comprises a nucleotide sequence that is complementary to a portion of an extension product formed from the first secondary oligonucleotide primer. The primary reverse-transcription/polymerase chain reaction products, the one or more secondary oligonucleotide primer sets, a deoxynucleotide mix, and a DNA polymerase are blended to form one or more first polymerase chain reaction mixtures, and the one or more first polymerase chain reaction mixtures are subjected to conditions suitable for two or more polymerase chain reaction cycles comprising a denaturation treatment, a hybridization treatment, and an extension treatment, thereby forming first polymerase chain reaction products comprising a 5′ primer-specific portion of the first secondary oligonucleotide primer, a nucleotide sequence corresponding to the target miRNA molecule sequence or a complement thereof, and a complement of the other 5′ primer-specific portion second secondary oligonucleotide primer. The method further involves providing one or more tertiary oligonucleotide primer sets. Each tertiary oligonucleotide primer set comprises (a) a first tertiary oligonucleotide primer comprising the same nucleotide sequence as the 5′ primer-specific portion of the first polymerase chain reaction products or their complements and (b) a second tertiary oligonucleotide primer comprising a nucleotide sequence that is complementary to the 3′ primer-specific portion of the first polymerase chain reaction products or their complements. The first polymerase chain reaction process products, the one or more tertiary oligonucleotide primer sets, the one or more enzymes capable of digesting deoxyuracil (dU)-containing nucleic acid molecules, a deoxynucleotide mix including dUTP, and a DNA polymerase are blended to form one or more second polymerase chain reaction mixtures, and the one or more second polymerase chain reaction mixtures are subjected to conditions suitable for digesting deoxyuracil (dU) containing nucleic acid molecules present in the second polymerase chain reaction mixtures and for carrying out one or more polymerase chain reaction cycles comprising a denaturation treatment, a hybridization treatment, and an extension treatment thereby forming second polymerase chain reaction products. The method further comprises detecting and distinguishing the second polymerase chain reaction products, thereby identifying one or more target miRNA molecules differing in sequence from other miRNA molecules in the sample by one or more bases.

Another aspect of the present application is directed to a method for identifying, in a sample, one or more target micro-ribonucleic acid (miRNA) molecules differing in sequence from other miRNA molecules in the sample by one or more bases. The method involves providing a sample containing one or more target miRNA molecules potentially differing in sequence from other miRNA molecules in the sample by one or more bases and providing one or more enzymes capable of digesting deoxyuracil (dU)-containing nucleic acid molecules present in the sample. The sample is contacted with one or more enzymes capable of digesting dU-containing nucleic acid molecules potentially present in the sample. The contacted sample is blended with ATP and a Poly(A) polymerase to form a Poly(A) polymerase reaction mixture, and the Poly(A) polymerase reaction mixture is subjected to conditions suitable for appending homopolymer A to the 3′ ends of the one or more target miRNA molecules potentially present in the sample. The method further involves providing one or more primary oligonucleotide primer sets. Each primer set comprises (a) a first primary oligonucleotide primer comprising a 5′ primer-specific portion, an internal poly dT portion, and a 3′ portion comprising from 1 to 10 bases complementary to the 3′ end of the target miRNA, wherein the first primary oligonucleotide primer may be the same or may differ from other first primary oligonucleotide primers in other sets, and (b) a second primary oligonucleotide primer comprising a 5′ primer-specific portion and a 3′ portion, wherein the second primary oligonucleotide primer may be the same or may differ from other second primary oligonucleotide primers in other sets. The Poly(A) polymerase reaction mixture, the one or more primary oligonucleotide primer sets, the one or more enzymes capable of digesting deoxyuracil (dU)-containing nucleic acid molecules in the sample, a deoxynucleotide mix including dUTP, and a reverse transcriptase and a DNA polymerase or a DNA polymerase with reverse-transcriptase activity are blended to form one or more reverse-transcription/polymerase chain reaction mixtures. The one or more reverse-transcription/polymerase chain reaction mixtures are subjected to conditions suitable for digesting deoxyuracil (dU)-containing nucleic acid molecules present in the reverse-transcription/polymerase chain reaction mixtures, then to conditions suitable for generating complementary deoxyribonucleic acid (cDNA) molecules to the target miRNA sequences with 3′ polyA tails, and to one or more polymerase chain reaction cycles comprising a denaturation treatment, a hybridization treatment, and an extension treatment thereby forming one or more different reverse-transcription/polymerase chain reaction products comprising the 5′ primer-specific portion of the second primary oligonucleotide primer, a nucleotide sequence corresponding to the target miRNA molecule sequence, a poly dA region, and the complement of the 5′ primer-specific portion of the first primary oligonucleotide primer, and complements thereof. The method further comprises providing one or more oligonucleotide probe sets. Each probe set comprises (a) a first oligonucleotide probe having a 5′ primer-specific portion and a 3′ target sequence-specific portion, and (b) a second oligonucleotide probe having a 5′ target sequence-specific portion, a portion complementary to the one or more reverse-transcription/polymerase chain reaction products, and a 3′ primer-specific portion, wherein the first and second oligonucleotide probes of a probe set are configured to hybridize, in a base specific manner, to complementary portions of the one or more reverse-transcription/polymerase chain reaction products corresponding to the target miRNA molecule sequence, or complement thereof. The one or more reverse-transcription/polymerase chain reaction products are contacted with a ligase and the one or more oligonucleotide probe sets to form one or more ligation reaction mixtures, and the one or more ligation reaction mixtures are subjected to one or more ligation reaction cycles whereby the first and second oligonucleotide probes of the one or more oligonucleotide probe sets, when hybridized to their complement, are ligated together to form ligated product sequences in the ligation reaction mixture, wherein each ligated product sequence comprises the 5′ primer-specific portion, the target-specific portions, and the 3′ primer-specific portion. The method further involves providing one or more secondary oligonucleotide primer sets. Each secondary oligonucleotide primer set comprises (a) a first secondary oligonucleotide primer comprising the same nucleotide sequence as the 5′ primer-specific portion of the ligated product sequence and (b) a second secondary oligonucleotide primer comprising a nucleotide sequence that is complementary to the 3′ primer-specific portion of the ligated product sequence. The ligated product sequences and the one or more secondary oligonucleotide primer sets are blended with one or more enzymes capable of digesting deoxyuracil (dU)-containing nucleic acid molecules, a deoxynucleotide mix including dUTP, and a DNA polymerase to form one or more first polymerase chain reaction mixtures, and the one or more first polymerase chain reaction mixtures are subjected to conditions suitable for digesting deoxyuracil (dU)-containing nucleic acid molecules present in the first polymerase chain reaction mixtures and for carrying out one or more polymerase chain reaction cycles comprising a denaturation treatment, a hybridization treatment, and an extension treatment thereby forming secondary polymerase chain reaction products. The method further comprises detecting and distinguishing the secondary polymerase chain reaction products, thereby identifying one or more target miRNA molecules differing in sequence from other miRNA molecules in the sample by one or more bases.

Another aspect of the present application is directed to a method for identifying, in a sample, one or more target micro-ribonucleic acid (miRNA) molecules differing in sequence from other miRNA molecules in the sample by one or more bases. The method involves providing a sample containing one or more target miRNA molecules potentially differing in sequence from other miRNA molecules in the sample by one or more bases and providing one or more enzymes capable of digesting deoxyuracil (dU)-containing nucleic acid molecules present in the sample. The sample is contacted with one or more enzymes capable of digesting dU-containing nucleic acid molecules potentially present in the sample. The contacted sample is blended with ATP and a Poly(A) polymerase to form a Poly(A) polymerase reaction mixture, and the Poly(A) polymerase reaction mixture is subjected to conditions suitable for appending a homopolymer A to the 3′ ends of the one or more target miRNA molecules potentially present in the sample. The method further involves providing one or more primary oligonucleotide primer sets. Each primer set comprises (a) a first primary oligonucleotide primer comprising a 5′ primer-specific portion, an internal poly dT portion, and a 3′ portion comprising from 1 to 10 bases complementary to the 3′ end of the target miRNA, wherein the first primary oligonucleotide primer may be the same or may differ from other first primary oligonucleotide primers in other sets, and (b) a second primary oligonucleotide primer comprising a 5′ primer-specific portion and a 3′ portion, wherein the second primary oligonucleotide primer may be the same or may differ from other second primary oligonucleotide primers in other sets. The Poly(A) polymerase reaction mixture potentially comprising target miRNA sequences is blended with 3′ polyA tails, the one or more primary oligonucleotide primer sets, a deoxynucleotide mix, and a reverse transcriptase and a DNA polymerase or a DNA polymerase with reverse-transcriptase activity to form one or more reverse-transcription/polymerase chain reaction mixtures. The one or more reverse-transcription/polymerase chain reaction mixtures are subjected to conditions suitable for generating complementary deoxyribonucleic acid (cDNA) molecules to the target miRNA sequences with 3′ polyA tails, and to one or more polymerase chain reaction cycles comprising a denaturation treatment, a hybridization treatment, and an extension treatment, thereby forming one or more different reverse-transcription/polymerase chain reaction products comprising the 5′ primer-specific portion of the second primary oligonucleotide primer, a nucleotide sequence corresponding to the target miRNA molecule sequence, a poly dA region, and the complement of the 5′ primer-specific portion of the first primary oligonucleotide primer, and complements thereof. The method further comprises providing one or more secondary oligonucleotide primer sets. Each secondary oligonucleotide primer set comprises (a) a first secondary oligonucleotide primer having a 5′ primer-specific portion and a 3′ portion that is complementary to a portion of a reverse-transcription/polymerase chain reaction product formed from the first primary oligonucleotide primer and (b) a second secondary oligonucleotide primer having a 5′ primer-specific portion and a 3′ portion that comprises a nucleotide sequence that is complementary to a portion of a reverse-transcription/polymerase chain reaction product formed from the first secondary oligonucleotide primer. The reverse-transcription/polymerase chain reaction products, the one or more secondary oligonucleotide primer sets, a deoxynucleotide mix, and a DNA polymerase are blended to form one or more first polymerase chain reaction mixtures, and the one or more first polymerase chain reaction mixtures are subjected to conditions suitable for two or more polymerase chain reaction cycles comprising a denaturation treatment, a hybridization treatment, and an extension treatment, thereby forming first polymerase chain reaction products comprising a 5′ primer-specific portion, a nucleotide sequence corresponding to the target miRNA molecule sequence or a complement thereof, and a complement of the other 5′ primer-specific portion. The method further involves providing one or more tertiary oligonucleotide primer sets. Each tertiary oligonucleotide primer set comprises (a) a first tertiary oligonucleotide primer comprising the same nucleotide sequence as the 5′ primer-specific portion of the first polymerase chain reaction product sequence and (b) a second tertiary oligonucleotide primer comprising a nucleotide sequence that is complementary to the 3′ primer-specific portion of the first polymerase chain reaction product sequence. The first polymerase chain reaction products, the one or more tertiary oligonucleotide primer sets, the one or more enzymes capable of digesting deoxyuracil (dU) containing nucleic acid molecules, a deoxynucleotide mix including dUTP, and a DNA polymerase are blended to form one or more second polymerase chain reaction mixtures. The one or more second polymerase chain reaction mixtures are subjected to conditions suitable for digesting deoxyuracil (dU)-containing nucleic acid molecules present in the first polymerase chain reaction mixtures, and one or more polymerase chain reaction cycles comprising a denaturation treatment, a hybridization treatment, and an extension treatment thereby forming second polymerase chain reaction products. The method further comprises detecting and distinguishing the second polymerase chain reaction products in the one or more reactions thereby identifying one or more target miRNA molecules differing in sequence from other miRNA molecules in the sample by one or more bases.

Another aspect of the present application is directed to a method of diagnosing or prognosing a disease state of cells or tissue based on identifying the presence or level of a plurality of disease-specific and/or cell/tissue-specific DNA, RNA, and/or protein markers in a biological sample of an individual. The plurality of markers is in a set comprising from 6-12 markers, 12-24 markers, 24-36 markers, 36-48 markers, 48-72 markers, 72-96 markers, or >96 markers. Each marker in a given set is selected by having any one or more of the following criteria: present, or above a cutoff level, in >50% of biological samples of the disease cells or tissue from individuals diagnosed with the disease state; absent, or below a cutoff level, in >95% of biological samples of the normal cells or tissue from individuals without the disease state; present, or above a cutoff level, in >50% of biological samples comprising cells, serum, blood, plasma, amniotic fluid, sputum, urine, bodily fluids, bodily secretions, bodily excretions, or fractions thereof, from individuals diagnosed with the disease state; absent, or below a cutoff level, in >95% of biological samples comprising cells, serum, blood, plasma, amniotic fluid, sputum, urine, bodily fluids, bodily secretions, bodily excretions, or fractions thereof, from individuals without the disease state; present with a z-value of >1.65 in the biological sample comprising cells, serum, blood, plasma, amniotic fluid, sputum, urine, bodily fluids, bodily secretions, bodily excretions, or fractions thereof, from individuals diagnosed with the disease state. At least 50% of the markers in a set each comprise one or more methylated residues, and/or at least 50% of the markers in a set that are present, or above a cutoff level, or present with a z-value of >1.65, comprise of one or more methylated residues, in the biological sample comprising cells, serum, blood, plasma, amniotic fluid, sputum, urine, bodily fluids, bodily secretions, bodily excretions, or fractions thereof, from at least 50% of individuals diagnosed with the disease state. The method involves obtaining a biological sample. The biological sample includes cell-free DNA, RNA, and/or protein originating from the cells or tissue and from one or more other tissues or cells, and the biological sample is selected from the group consisting of cells, serum, blood, plasma, amniotic fluid, sputum, urine, bodily fluids, bodily secretions, and bodily excretions, or fractions thereof. The sample is fractionated into one or more fractions, wherein at least one fraction comprises exosomes, tumor-associated vesicles, other protected states, or cell-free DNA, RNA, and/or protein. Nucleic acid molecules in the one or more fractions are subjected to a bisulfite treatment under conditions suitable to convert unmethylated cytosine residues to uracil residues. At least two enrichment steps are carried out for 50% or more disease-specific and/or cell/tissue-specific DNA, RNA, and/or protein markers during either said fractionating and/or by carrying out a nucleic acid amplification step. The method further involves performing one or more assays to detect and distinguish the plurality of disease-specific and/or cell/tissue-specific DNA, RNA, and/or protein markers, thereby identifying their presence or levels in the sample, wherein individuals are diagnosed or prognosed with the disease state if a minimum of 2 or 3 markers are present or are above a cutoff level in a marker set comprising from 6-12 markers; or a minimum of 3, 4, or 5 markers are present or are above a cutoff level in a marker set comprising from 12-24 markers; or a minimum of 3, 4, 5, or 6 markers are present or are above a cutoff level in a marker set comprising from 24-36 markers; or a minimum of 4, 5, 6, 7, or 8 markers are present or are above a cutoff level in a marker set comprising from 36-48 markers; or a minimum of 6, 7, 8, 9, 10, 11, or 12 markers are present or are above a cutoff level in a marker set comprising from 48-72 markers, or a minimum of 7, 8, 9, 10, 11, 12 or 13 markers are present or are above a cutoff level in a marker set comprising from 72-96 markers, or a minimum of 8, 9, 10, 11, 12, 13 or “n”/12 markers are present or are above a cutoff level in a marker set comprising 96−“n” markers, when “n”>168 markers.

Another aspect of the present application is directed to a method of diagnosing or prognosing a disease state of a solid tissue cancer including colorectal adenocarcinoma, stomach adenocarcinoma, esophageal carcinoma, breast lobular and ductal carcinoma, uterine corpus endometrial carcinoma, ovarian serous cystadenocarcinoma, cervical squamous cell carcinoma and adenocarcinoma, uterine carcinosarcoma, lung adenocarcinoma, lung squamous cell carcinoma, head & neck squamous cell carcinoma, prostate adenocarcinoma, invasive urothelial bladder cancer, liver hepatoceullular carcinoma, pancreatic ductal adenocarcinoma, or gallbladder adenocarcinoma based on identifying the presence or level of a plurality of disease-specific and/or cell/tissue-specific DNA, RNA, and/or protein markers in a biological sample of an individual. The plurality of markers is in a set comprising from 48-72 total cancer markers, 72-96 total cancer markers or ≥96 total cancer markers, wherein on average greater than one quarter such markers in a given set cover each of the aforementioned major cancers being tested. Each marker in a given set for a given solid tissue cancer is selected by having any one or more of the following criteria for that solid tissue cancer: present, or above a cutoff level, in >50% of biological samples of a given cancer tissue from individuals diagnosed with a given solid tissue cancer; absent, or below a cutoff level, in >95% of biological samples of the normal tissue from individuals without that given solid tissue cancer; present, or above a cutoff level, in >50% of biological samples comprising cells, serum, blood, plasma, amniotic fluid, sputum, urine, bodily fluids, bodily secretions, bodily excretions, or fractions thereof, from individuals diagnosed with a given solid tissue cancer; absent, or below a cutoff level, in >95% of biological samples comprising cells, serum, blood, plasma, amniotic fluid, sputum, urine, bodily fluids, bodily secretions, bodily excretions, or fractions thereof, from individuals without that given solid tissue cancer; present with a z-value of >1.65 in the biological sample comprising cells, serum, blood, plasma, amniotic fluid, sputum, urine, bodily fluids, bodily secretions, bodily excretions, or fractions thereof, from individuals diagnosed with a given solid tissue cancer. At least 50% of the markers in a set each comprise one or more methylated residues, and/or at least 50% of the markers in a set that are present, or above a cutoff level, or present with a z-value of >1.65, comprise of one or more methylated residues, in the biological sample comprising cells, serum, blood, plasma, amniotic fluid, sputum, urine, bodily fluids, bodily secretions, bodily excretions, or fractions thereof, from at least 50% of individuals diagnosed with a given solid tissue cancer. The method involves obtaining a biological sample including cell-free DNA, RNA, and/or protein originating from the cells or tissue and from one or more other tissues or cells, wherein the biological sample is selected from the group consisting of cells, serum, blood, plasma, amniotic fluid, sputum, urine, bodily fluids, bodily secretions, and bodily excretions, or fractions thereof. The sample is fractionated into one or more fractions, wherein at least one fraction comprises exosomes, tumor-associated vesicles, other protected states, or cell-free DNA, RNA, and/or protein. The nucleic acid molecules in the one or more fractions are subjected to a bisulfite treatment under conditions suitable to convert unmethylated cytosine residues to uracil residues. At least two enrichment steps are carried out for 50% or more of the given solid tissue cancer-specific and/or cell/tissue-specific DNA, RNA, and/or protein markers during either said fractionating and/or by carrying out a nucleic acid amplification step. The method further involves performing one or more assays to detect and distinguish the plurality of cancer-specific and/or cell/tissue-specific DNA, RNA, and/or protein markers, thereby identifying their presence or levels in the sample, wherein individuals are diagnosed or prognosed with the a solid-tissue cancer if a minimum of 4 markers are present or are above a cutoff level in a marker set comprising from 48-72 total cancer markers; or a minimum of 5 markers are present or are above a cutoff level in a marker set comprising from 72-96 total cancer markers; or a minimum of 6 or “n”/18 markers are present or are above a cutoff level in a marker set comprising 96 to “n” total cancer markers, when “n”>96 total cancer markers.

Another aspect of the present application is directed to a method of diagnosing or prognosing a disease state of and identifying the most likely specific tissue(s) of origin of a solid tissue cancer in the following groups: Group 1 (colorectal adenocarcinoma, stomach adenocarcinoma, esophageal carcinoma); Group 2 (breast lobular and ductal carcinoma, uterine corpus endometrial carcinoma, ovarian serous cystadenocarcinoma, cervical squamous cell carcinoma and adenocarcinoma, uterine carcinosarcoma); Group 3 (lung adenocarcinoma, lung squamous cell carcinoma, head & neck squamous cell carcinoma); Group 4 (prostate adenocarcinoma, invasive urothelial bladder cancer); and/or Group 5 (liver hepatoceullular carcinoma, pancreatic ductal adenocarcinoma, or gallbladder adenocarcinoma) based on identifying the presence or level of a plurality of disease-specific and/or cell/tissue-specific DNA, RNA, and/or protein markers in a biological sample of an individual, wherein the plurality of markers is in a set comprising from 36-48 group-specific cancer markers, 48-64 group-specific cancer markers, or 64 group-specific cancer markers, wherein on average greater than one third of such markers in a given set cover each of the aforementioned cancers being tested within that group. Each marker in a given set for a given solid tissue cancer is selected by having any one or more of the following criteria for that solid tissue cancer: present, or above a cutoff level, in >50% of biological samples of a given cancer tissue from individuals diagnosed with a given solid tissue cancer; absent, or below a cutoff level, in >95% of biological samples of the normal tissue from individuals without that given solid tissue cancer; present, or above a cutoff level, in >50% of biological samples comprising cells, serum, blood, plasma, amniotic fluid, sputum, urine, bodily fluids, bodily secretions, bodily excretions, or fractions thereof, from individuals diagnosed with a given solid tissue cancer; absent, or below a cutoff level, in >95% of biological samples comprising cells, serum, blood, plasma, amniotic fluid, sputum, urine, bodily fluids, bodily secretions, bodily excretions, or fractions thereof, from individuals without that given solid tissue cancer; present with a z-value of >1.65 in the biological sample comprising cells, serum, blood, plasma, amniotic fluid, sputum, urine, bodily fluids, bodily secretions, bodily excretions, or fractions thereof, from individuals diagnosed with a given solid tissue cancer. At least 50% of the markers in a set each comprise one or more methylated residues, and/or at least 50% of the markers in a set that are present, or above a cutoff level, or present with a z-value of >1.65 comprise one or more methylated residues, in the biological sample comprising cells, serum, blood, plasma, amniotic fluid, sputum, urine, bodily fluids, bodily secretions, bodily excretions, or fractions thereof, from at least 50% of individuals diagnosed with a given solid tissue cancer. The method involves obtaining the biological sample. The biological sample includes cell-free DNA, RNA, and/or protein originating from the cells or tissue and from one or more other tissues or cells, wherein the biological sample is selected from the group consisting of cells, serum, blood, plasma, amniotic fluid, sputum, urine, bodily fluids, bodily secretions, and bodily excretions, or fractions thereof. The sample is fractionated into one or more fractions, wherein at least one fraction comprises exosomes, tumor-associated vesicles, other protected states, or cell-free DNA, RNA, and/or protein. The nucleic acid molecules in the one or more fractions are subjected to a bisulfite treatment under conditions suitable to convert unmethylated cytosine residues to uracil residues. At least two enrichment steps are carried out for 50% or more of the given solid tissue cancer-specific and/or cell/tissue-specific DNA, RNA, and/or protein markers during either said fractionating and/or by carrying out a nucleic acid amplification step. The method further involves performing one or more assays to detect and distinguish the plurality of cancer-specific and/or cell/tissue-specific DNA, RNA, and/or protein markers, thereby identifying their presence or levels in the sample, wherein individuals are diagnosed or prognosed with a solid-tissue cancer if a minimum of 4 markers are present or are above a cutoff level in a marker set comprising from 36-48 group-specific cancer markers; or a minimum of 5 markers are present or are above a cutoff level in a marker set comprising from 48-64 group-specific cancer markers; or a minimum of 6 or “n”/12 markers are present or are above a cutoff level in a marker set comprising 64 to “n” total cancer markers, when “n”>64 group-specific cancer markers.

The present application describes a number of approaches for detecting mutations, expression, splice variant, translocation, copy number, and/or methylation changes in target nucleic acid molecules using nuclease, ligase, and polymerase reactions. The present application solves the problems of carry over prevention, as well as allowing for spatial multiplexing to provide relative quantification, similar to digital PCR. Such technology may be utilized for non-invasive early detection of cancer, non-invasive prognosis of cancer, and monitoring for cancer recurrence from plasma or serum samples.

The present application provides a comprehensive roadmap of nucleic acid methylation, miRNA, lncRNA, ncRNA, mRNA Exons, as well as cancer-associated protein markers that are specific for solid-tissue cancers and matched normal tissues. The present application teaches the art of selecting the desired number of markers and types of markers for both pan-oncology and specific cancers (i.e. colorectal cancer) to guide the physician to improve the treatment of the patient. Details on primer design and optimized primer sequences are provided to enable rapid validation of these tests for both pan-oncology and specific cancers. The two-step procedure is designed to cast a wide net to initially identify most of the individuals harboring an early cancer, followed by a more stringent second step to improve specificity and narrow the patients to those most likely to harbor a hidden cancer, who are then sent for imaging and followup. The advantage of this 2-step approach is that it not only identifies the potential tissue of origin, but it is designed to provide the highest positive predictive value (PPV). Thus, when a result for a rare cancer comes back as presumptive positive (i.e. early ovarian cancer) the physician can focus her attention on providing imaging and followup to those patients who need it the most, while the test minimizes the false-positives that create unnecessary anxiety and unwanted invasive procedures.

The present application provides robust approaches for detecting markers of cancer (mutations, expression, splice variant, translocation, copy number, and/or methylation changes) using either qPCR or dPCR readout using protocols that are amenable to automation and work on readily available commercial instruments. The approach provides advantages in being integrated and convenient for laboratory setup, allowing for cost reduction, scalability, and fit with medical and laboratory flow in a CLIA-compatible automated setting. The benefit in lives saved world-wide would be of incalculable value.

### BRIEF DESCRIPTION OF THE DRAWINGS

FIG. 1A-B illustrates a conditional logic tree for an early detection colorectal cancer test based on analysis of a patient's blood sample. FIG. 1A illustrates a one-step colorectal cancer assay using 24 markers at average sensitivity of 50%. FIG. 1B illustrates a two-step colorectal cancer assay using 24 markers in the first step at average sensitivity of 50%, and 48 markers in a second step. FIGS. 1C-D illustrate a conditional logic tree for a two-step assay for early detection pan-oncology pan-oncology cancer test based on analysis of a patient's blood sample. FIG. 1C illustrates a two-step pan-oncology assay using 96 group-specific markers at average sensitivity of 50% in the first step, followed by 1 or 2 groups of 64 type-specific markers each at average sensitivity of 50% in the second step. FIG. 1D illustrates a two-step pan-oncology assay using 96 group-specific markers at average sensitivity of 66% in the first step, followed by 1 or 2 groups of 64 type-specific markers each at average sensitivity of 66% in the second step.

FIG. 2 illustrates exPCR-LDR-qPCR carryover prevention reaction with Taqman™ detection to identify or relatively quantify target(s) and/or low-level mutations.

FIG. 3 illustrates exPCR-LDR-qPCR carryover prevention reaction with UniTaq detection to identify or relatively quantify target(s) and/or low-level mutations.

FIG. 4 illustrates exPCR-qPCR carryover prevention reaction with Taqman™ detection to identify or relatively quantify target(s) and/or low-level mutations.

FIG. 5 illustrates exPCR-qPCR carryover prevention reaction with UniTaq detection to identify or relatively quantify target(s) and/or low-level mutations.

FIG. 6 illustrates a variation of exPCR-qPCR carryover prevention reaction with Taqman™ detection to identify or relatively quantify target(s) and/or low-level mutations.

FIG. 7 illustrates another variation of exPCR-qPCR carryover prevention reaction with Taqman™ detection to identify or relatively quantify target(s) and/or low-level mutations.

FIG. 8 illustrates a variation of exPCR-qPCR carryover prevention reaction with UniTaq detection to identify or relatively quantify target(s) and/or low-level mutations.

FIG. 9 illustrates exPCR-LDR-qPCR carryover prevention reaction with Taqman™ detection to identify or relatively quantify low-level methylation.

FIG. 10 illustrates a variation of exPCR-LDR-qPCR carryover prevention reaction with Taqman™ detection to identify or relatively quantify low-level methylation.

FIG. 11 illustrates exPCR-qPCR carryover prevention reaction with Taqman™ detection to identify or relatively quantify low-level methylation.

FIG. 12 illustrates a variation of exPCR-qPCR carryover prevention reaction with Taqman™ detection to identify or relatively quantify low-level methylation.

FIG. 13 illustrates another variation of exPCR-qPCR carryover prevention reaction with Taqman™ detection to identify or relatively quantify low-level methylation.

FIG. 14 illustrates another variation of exPCR-qPCR carryover prevention reaction with Taqman™ detection to identify or relatively quantify low-level methylation.

FIG. 15 illustrates another variation of exPCR-qPCR carryover prevention reaction with Taqman™ detection to identify or relatively quantify low-level methylation.

FIG. 16 illustrates another variation of exPCR-LDR-qPCR carryover prevention reaction with Taqman™ detection to identify or relatively quantify low-level methylation.

FIG. 17 illustrates another variation of exPCR-LDR-qPCR carryover prevention reaction with Taqman™ detection to identify or relatively quantify low-level methylation.

FIG. 18 illustrates another variation of exPCR-qPCR carryover prevention reaction with Taqman™ detection to identify or relatively quantify low-level methylation.

FIG. 19 illustrates another variation of exPCR-qPCR carryover prevention reaction with Taqman™ detection to identify or relatively quantify low-level methylation.

FIG. 20 illustrates another variation of exPCR-qPCR carryover prevention reaction with Taqman™ detection to identify or relatively quantify low-level methylation.

FIG. 21 illustrates another variation of exPCR-qPCR carryover prevention reaction with Taqman™ detection to identify or relatively quantify low-level methylation.

FIG. 22 illustrates another variation of exPCR-qPCR carryover prevention reaction with Taqman™ detection to identify or relatively quantify low-level methylation.

FIG. 23 illustrates RT-PCR-LDR-qPCR carryover prevention reaction with Taqman™ detection to detect and enumerate translocation events at the mRNA level.

FIG. 24 illustrates RT-PCR-qPCR carryover prevention reaction with Taqman™ detection to detect and enumerate translocation events at the mRNA level.

FIG. 25 illustrates RT-PCR-PCR-qPCR carryover prevention reaction with Taqman™ detection to detect and enumerate translocation events at the mRNA level.

FIG. 26 illustrates RT-PCR-LDR-qPCR carryover prevention reaction with Taqman™ detection to detect and enumerate RNA copy number.

FIG. 27 illustrates RT-PCR-qPCR carryover prevention reaction with Taqman™ detection to detect and enumerate RNA copy number.

FIG. 28 illustrates RT-PCR-PCR-qPCR carryover prevention reaction with Taqman™ detection to detect and enumerate RNA copy number.

FIG. 29 illustrates Ligation-RT-PCR-LDR-qPCR carryover prevention reaction with Taqman™ detection to detect and enumerate miRNA.

FIG. 30 illustrates Ligation-RT-PCR-qPCR carryover prevention reaction with Taqman™ detection to detect and enumerate miRNA.

FIG. 31 illustrates RT-PCR-LDR-qPCR carryover prevention reaction with Taqman™ detection to detect and enumerate miRNA.

FIG. 32 illustrates RT-PCR-qPCR carryover prevention reaction with Taqman™ detection to detect and enumerate miRNA.

FIGS. 33A-B illustrate results for calculated overall Sensitivity and Specificity for a 24-marker assay, where the average individual marker sensitivity is 50% (FIG. 33A), and the average individual marker false-positive rate is from 2% to 5% (FIG. 33B).

FIGS. 34A-B illustrate results for calculated overall Sensitivity and Specificity for a 36-marker assay, where the average individual marker sensitivity is 50% (FIG. 34A), and the average individual marker false-positive rate is from 2% to 5% (FIG. 34B).

FIGS. 35A-B illustrate results for calculated overall Sensitivity and Specificity for a 48-marker assay, where the average individual marker sensitivity is 50% (FIG. 35A), and the average individual marker false-positive rate is from 2% to 5% (FIG. 35B).

FIGS. 36A-B illustrate results for calculated overall Sensitivity and Specificity for a 96-marker assay, where the average individual marker sensitivity is 50% (FIG. 36A), and the average individual marker false-positive rate is from 2% to 5% (FIG. 36B).

FIGS. 37A-B illustrate the ROC curve for a 48-marker assay, where the average individual marker sensitivity is 50%, as well as the calculated AUC, when the average number of molecules per marker in the blood ranges from 150 to 600 molecules. For FIGS. 37A and 37B, the calculations are based on an average individual marker false-positive rate of 2% and 3%, respectively.

FIGS. 38A-B illustrate the ROC curve for a 48-marker assay, where the average individual marker sensitivity is 50%, as well as the calculated AUC, when the average number of molecules per marker in the blood ranges from 150 to 600 molecules. For FIGS. 38A and 38B, the calculations are based on an average individual marker false-positive rate of 4% and 5%, respectively.

FIGS. 39A-B provide a list of blood-based, colon cancer-specific microRNA markers derived through analysis of TCGA microRNA datasets, which may be present in exosomes or other protected state in the blood.

FIGS. 40A-X provide a list of blood-based, colon cancer-specific ncRNA and lncRNA markers, which may be present in exosomes or other protected state in the blood.

FIGS. 41A-C provide a list of candidate blood-based colon cancer-specific exon transcripts that may be enriched in in exosomes or other protected state in the blood.

FIGS. 42A-J provide a list of cancer proteins markers, identified through, mRNA sequences, protein expression levels, protein product concentrations, cytokines, or autoantibody to the protein product arising from Colorectal tumors, which may be identified in the blood, either within exosomes, other protected states, tumor-associated vesicles, or free within the plasma.

FIG. 43 provides a list of protein markers that can be secreted by Colorectal tumors into the blood.

FIGS. 44A-Y provide a list of primary CpG sites that are Colorectal Cancer and Colon-tissue specific markers, that may be used to identify the presence of colorectal cancer from cfDNA, or DNA within exosomes, or DNA in another protected state (such as within CTCs) within the blood.

FIGS. 45A-P provide a list of chromosomal regions or sub-regions within which are primary CpG sites that are Colorectal Cancer and Colon-tissue specific markers, that may be used to identify the presence of colorectal cancer from cfDNA, or DNA within exosomes, or DNA in other protected state (such as within CTCs) within the blood.

FIGS. 46A-B illustrate results for calculated overall Sensitivity and Specificity for a 24-marker assay, where the average individual marker sensitivity is 50%, and the average individual marker false-positive rate is from 2% to 5%; including one marker with a sensitivity at 90% (FIG. 46A) and a 10% (FIG. 46B) false-positive rate.

FIGS. 47A-B illustrate results for calculated overall Sensitivity and Specificity for a 24-marker assay, where the average individual marker sensitivity is 50%, and the average individual marker false-positive rate is from 2% to 5%; including two markers with a sensitivity at 90% (FIG. 47A) and a 10% (FIG. 47B) false-positive rate.

FIGS. 48A-B illustrate results for calculated overall Sensitivity and Specificity for a 48-marker assay, where the average individual marker sensitivity is 50%, and the average individual marker false-positive rate is from 2% to 5%; including one marker with a sensitivity at 90% (FIG. 48A) and a 10% (FIG. 48B) false-positive rate.

FIGS. 49A-B illustrate results for calculated overall Sensitivity and Specificity for a 48-marker assay, where the average individual marker sensitivity is 50%, and the average individual marker false-positive rate is from 2% to 5%; including two markers with a sensitivity at 90% (FIG. 49A) and a 10% (FIG. 49B) false-positive rate.

FIGS. 50A-B illustrate results for calculated overall Sensitivity and Specificity for a 24-marker assay, where the average individual marker sensitivity is 66% (FIG. 50A), and the average individual marker false-positive rate is from 2% to 5% (FIG. 50B).

FIGS. 51A-B illustrate results for calculated overall Sensitivity and Specificity for a 36-marker assay, where the average individual marker sensitivity is 66% (FIG. 51A), and the average individual marker false-positive rate is from 2% to 5% (FIG. 51B).

FIGS. 52A-B illustrate results for calculated overall Sensitivity and Specificity for a 48-marker assay, where the average individual marker sensitivity is 66% (FIG. 52A), and the average individual marker false-positive rate is from 2% to 5% (FIG. 52B).

FIG. 53 provides a list of blood-based, solid tumor-specific ncRNA and lncRNA markers, which may be present in exosomes or other protected state in the blood.

FIGS. 54A-F provide a list of candidate blood-based solid tumor-specific exon transcripts that may be enriched in in exosomes or other protected state in the blood.

FIGS. 55A-H provide a list of cancer proteins markers, identified through, mRNA sequences, protein expression levels, protein product concentrations, cytokines, or autoantibody to the protein product arising from solid tumors, which may be identified in the blood, either within exosomes, other protected states, tumor-associated vesicles, or free within the plasma.

FIGS. 56A-S provide a list of primary CpG sites that are solid-tumor and tissue-specific markers, that may be used to identify the presence of solid-tumor cancer from cfDNA, or DNA within exosomes, or DNA in another protected state (such as within CTCs) within the blood.

FIGS. 57A-J provide a list of chromosomal regions or sub-regions within which are primary CpG sites that are solid-tumor and tissue-specific markers, that may be used to identify the presence of solid-tumor cancer from cfDNA, or DNA within exosomes, or DNA in another protected state (such as within CTCs) within the blood.

FIG. 58 provide a list of cancer proteins markers, identified through, mRNA sequences, protein expression levels, protein product concentrations, cytokines, or autoantibody to the protein product arising from colon adenocarcinoma, rectal adenocarcinoma, stomach adenocarcinoma, or esophageal carcinoma, which may be identified in the blood, either within exosomes, other protected states, tumor-associated vesicles, or free within the plasma.

FIGS. 59A-S provide a list of primary CpG sites that are colon adenocarcinoma, rectal adenocarcinoma, stomach adenocarcinoma, or esophageal carcinoma and tissue-specific markers, that may be used to identify the presence of solid-tumor cancer from cfDNA, or DNA within exosomes, or DNA in another protected state (such as within CTCs) within the blood.

FIGS. 60A-J provide a list of chromosomal regions or sub-regions within which are primary CpG sites that are colon adenocarcinoma, rectal adenocarcinoma, stomach adenocarcinoma, or esophageal carcinoma and tissue-specific markers, that may be used to identify the presence of solid-tumor cancer from cfDNA, or DNA within exosomes, or DNA in another protected state (such as within CTCs) within the blood.

FIGS. 61A-C provide a list of primary CpG sites that are breast lobular and ductal carcinoma, uterine corpus endometrial carcinoma, ovarian serous cystadenocarcinoma, cervical squamous cell carcinoma and adenocarcinoma, or uterine carcinosarcoma and tissue-specific markers, that may be used to identify the presence of solid-tumor cancer from cfDNA, or DNA within exosomes, or DNA in another protected state (such as within CTCs) within the blood.

FIGS. 62A-B provide a list of chromosomal regions or sub-regions within which are primary CpG sites that are breast lobular and ductal carcinoma, uterine corpus endometrial carcinoma, ovarian serous cystadenocarcinoma, cervical squamous cell carcinoma and adenocarcinoma, or uterine carcinosarcoma and tissue-specific markers, that may be used to identify the presence of solid-tumor cancer from cfDNA, or DNA within exosomes, or DNA in another protected state (such as within CTCs) within the blood.

FIG. 63 provides a list of primary CpG sites that are lung adenocarcinoma, lung squamous cell carcinoma, or head & neck squamous cell carcinoma and tissue-specific markers, that may be used to identify the presence of solid-tumor cancer from cfDNA, or DNA within exosomes, or DNA in another protected state (such as within CTCs) within the blood.

FIG. 64 provides a list of chromosomal regions or sub-regions within which are primary CpG sites that are lung adenocarcinoma, lung squamous cell carcinoma, or head & neck squamous cell carcinoma and tissue-specific markers, that may be used to identify the presence of solid-tumor cancer from cfDNA, or DNA within exosomes, or DNA in another protected state (such as within CTCs) within the blood.

FIG. 65 provides a list of primary CpG sites that are prostate adenocarcinoma or invasive urothelial bladder cancer and tissue-specific markers, that may be used to identify the presence of solid-tumor cancer from cfDNA, or DNA within exosomes, or DNA in another protected state (such as within CTCs) within the blood.

FIG. 66 provides a list of chromosomal regions or sub-regions within which are primary CpG sites that are prostate adenocarcinoma or invasive urothelial bladder cancer and tissue-specific markers, that may be used to identify the presence of solid-tumor cancer from cfDNA, or DNA within exosomes, or DNA in another protected state (such as within CTCs) within the blood.

FIG. 67 provides a list of blood-based, liver hepatoceullular carcinoma, pancreatic ductal adenocarcinoma, or gallbladder adenocarcinoma-specific ncRNA and lncRNA markers, which may be present in exosomes or other protected state in the blood.

FIGS. 68A-E provide a list of candidate blood-based liver hepatoceullular carcinoma, pancreatic ductal adenocarcinoma, or gallbladder adenocarcinoma-specific exon transcripts that may be enriched in exosomes or other protected state in the blood.

FIGS. 69A-B provide a list of cancer proteins markers, identified through, mRNA sequences, protein expression levels, protein product concentrations, cytokines, or autoantibody to the protein product arising from liver hepatoceullular carcinoma, pancreatic ductal adenocarcinoma, or gallbladder adenocarcinoma, which may be identified in the blood, either within exosomes, other protected states, tumor-associated vesicles, or free within the plasma.

FIGS. 70A-E provide a list of primary CpG sites that are liver hepatoceullular carcinoma, pancreatic ductal adenocarcinoma, or gallbladder adenocarcinoma and tissue-specific markers, that may be used to identify the presence of solid-tumor cancer from cfDNA, or DNA within exosomes, or DNA in another protected state (such as within CTCs) within the blood.

FIGS. 71A-C provide a list of chromosomal regions or sub-regions within which are primary CpG sites that are liver hepatoceullular carcinoma, pancreatic ductal adenocarcinoma, or gallbladder adenocarcinoma and tissue-specific markers, that may be used to identify the presence of solid-tumor cancer from cfDNA, or DNA within exosomes, or DNA in another protected state (such as within CTCs) within the blood.

FIG. 72 illustrates the real-time PCR amplification plots obtained in the pixel Bisulfite-PCR-LDR-qPCR experiments to enumerate single molecules of methylated DNA in the presence of an excess of unmethylated DNA (Roche DNA).

FIG. 73 illustrates the real-time PCR amplification plots obtained in a multiplexed detection of 10 CRC methylation markers by Bisulfite-PCR-LDR-qPCR, using HT29 cell line DNA, with an average of 20 molecules each marker in 10,000 molecules of normal, e.g. unmethylated DNA (Roche DNA).

FIG. 74 illustrates the real-time PCR amplification plots obtained in a multiplexed detection of 7 CRC methylation markers by Bisulfite-exPCR-LDR-qPCR, using HT29 cell line DNA, with an average of 30 molecules each marker in 3,000 molecules of normal, e.g. unmethylated DNA (Roche DNA).

FIGS. 75A-B illustrate the real-time PCR amplification plots obtained in a multiplexed detection of 7 CRC methylation markers by Bisulfite-exPCR-LDR-qPCR, using cfDNA isolated from CRC (FIG. 75A) and Normal (FIG. 75B) plasma.

FIGS. 76A-B illustrate the real-time PCR amplification plots obtained in a multiplexed detection of 7 CRC methylation markers by Bisulfite-exPCR-LDR-qPCR, using cfDNA isolated from CRC (FIG. 76A) and Normal (FIG. 76B) plasma.

FIGS. 77A-B illustrate the real-time PCR amplification plots obtained in a multiplexed detection of 7 CRC methylation markers by Bisulfite-exPCR-LDR-qPCR, using cfDNA isolated from CRC (FIG. 77A) and Normal (FIG. 77B) plasma.

FIGS. 78A-B illustrate the real-time PCR amplification plots obtained in a multiplexed detection of 20 CRC methylation markers by Bisulfite-exPCR-LDR-qPCR, using HT29 cell line DNA, with 1,500 genome equivalents of HT29 cell line DNA in 7,500 genome equivalents of normal, e.g. unmethylated DNA (Roche DNA; FIG. 78A) compared with 7,500 genome equivalents of normal, e.g. unmethylated DNA (FIG. 78B).

FIGS. 79A-B illustrate the real-time PCR amplification plots obtained in a multiplexed detection of 20 CRC methylation markers by Bisulfite-exPCR-LDR-qPCR, using reverse primers with tails, using HT29 cell line DNA, with 200 genome equivalents of HT29 cell line DNA in 7,500 genome equivalents of normal, e.g. unmethylated DNA (Roche DNA; FIG. 79A) compared with 7,500 genome equivalents of normal, e.g. unmethylated DNA (FIG. 79B).

FIGS. 80A-B illustrate the real-time PCR amplification plots obtained in a multiplexed detection of 20 CRC methylation markers by Bisulfite-exPCR-LDR-qPCR, using reverse primers without tails, using HT29 cell line DNA, with 200 genome equivalents of HT29 cell line DNA in 7,500 genome equivalents of normal, e.g. unmethylated DNA (Roche DNA;

FIG. 80A) compared with 7,500 genome equivalents of normal, e.g. unmethylated DNA (FIG. 80B).

### DETAILED DESCRIPTION

A Universal Design for Early Detection of Cancer Using “Cancer Marker Load”

The most cost-effective early cancer detection test may combine an initial multiplexed coupled amplification and ligation assay to determine “cancer load”. For cancer detection, this would achieve >95% sensitivity for all cancers (pan-oncology), at >97% specificity.

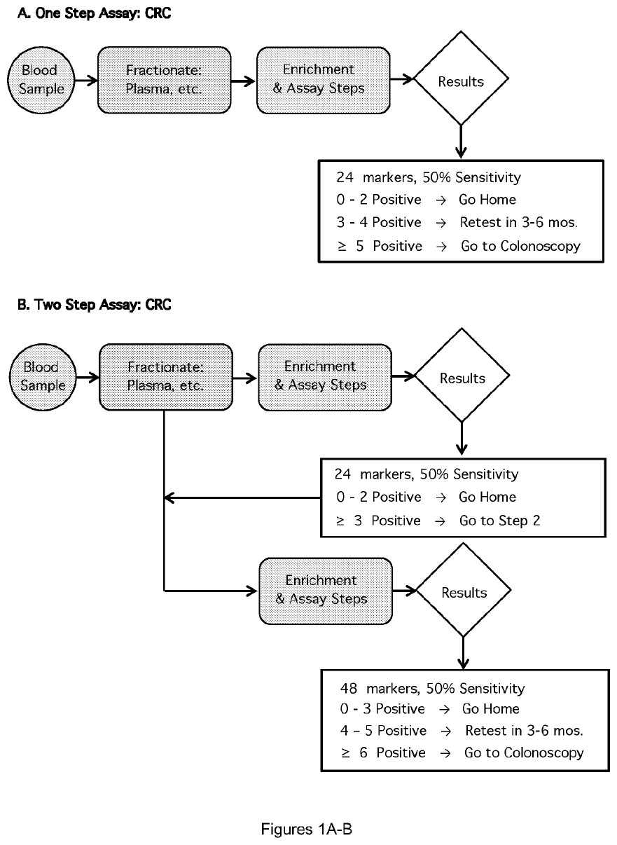

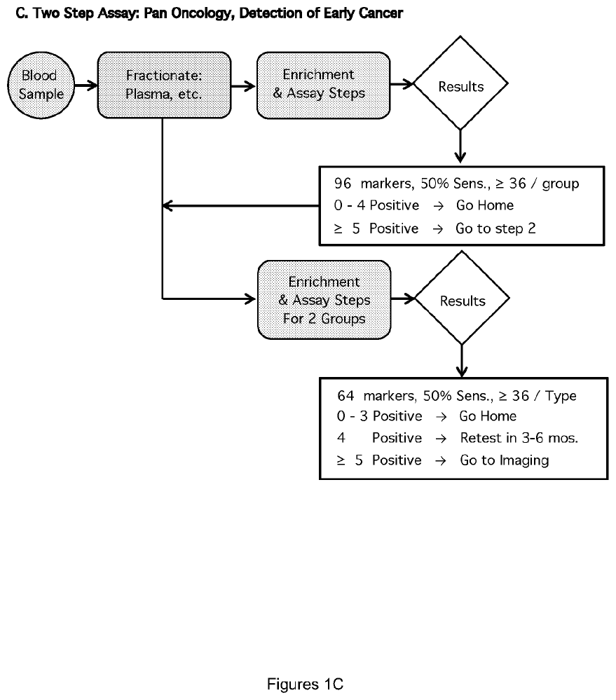

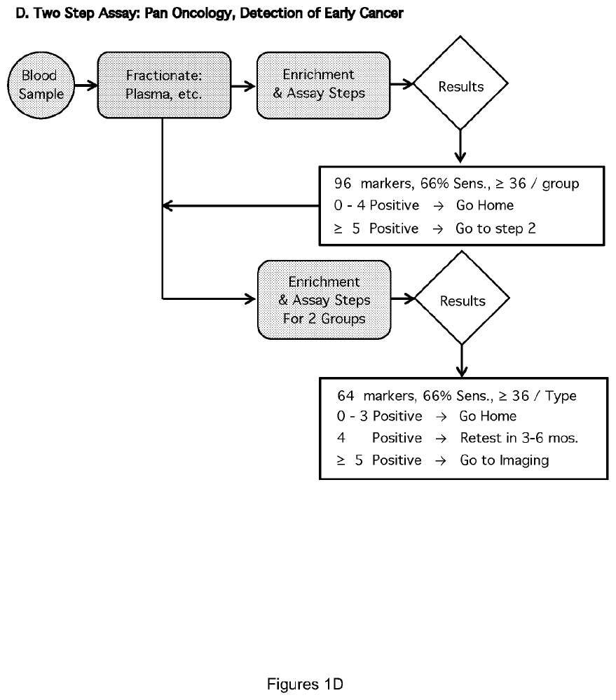

Several flow charts for a cancer tumor load assay is illustrated in FIG. 1. In its simplest form, the assay would be a one-step assay to identify individuals with early colorectal cancer (CRC). A blood sample is fractionated into plasma and other components as needed, a set of 24 markers with average sensitivity of 50% are assayed, and the results are recorded (FIG. 1A). For example, an initial multiplexed PCR/LDR screening assay scoring for mutation, methylation, miRNA, mRNA, alternative splicing, and/or translocations identifies those samples with positive results. The physician is not concerned with which specific markers are positive but gives a simple directive. Those patients with 0-2 markers positive are told not to worry, go home, you are cancer-free. Those patients with 5 of 24 markers positive are directed to get a colonoscopy. Those patients with an intermediate number of positive markers (3-4) are instructed to come back in 3-6 months for retesting. Thus, the test is based on the overall cancer marker load and not dependent on the specific markers that teat positive.

In an advanced version of the test, a two-step assay would be performed to identify if the patient has colorectal cancer. The rationale for a two-step test is to initially cast a wide net to maximize sensitivity in identifying the most individuals with potential cancer, followed by a second step only on the positive samples (which contain both true and false-positives) to maximize specificity, eliminate virtually all the false-positives, and hone in on those individuals most likely to have cancer. In the first step, a blood sample is fractionated into plasma and other components as needed, followed by an assay to interrogate an initial set of 24 markers with an average sensitivity of 50% (FIG. 1B). The first step assay can employ multiplexed PCR/LDR, or digital PCR screening to score for mutation, methylation, miRNA, mRNA, alternative splicing, and/or translocations events. As in the one-step assay, patients with 0-2 markers positive are presumed to be cancer-free. On the other hand, patients with 3 markers positive will undergo a second step, wherein 48 (new) markers are assayed and scored as follows: 0-3 positive markers are considered cancer-free; 4-5 positive markers are advised to come back in 3-6 months for retesting; 6 positive markers are directed to go get a colonoscopy.

In a pan-oncology version of the test, in the first step the assay would screen 96 markers, wherein on average 36 such markers would exhibit an average sensitivity of 50% for most major cancers (see FIG. 1C). These cancers would cluster to certain groups, which include: Group 1 (Colorectal, Stomach, Esophagus); Group 2 (Breast, Endometrial, Ovarian, Cervical, Uterine); Group 3 (Lung, Head & Neck); Group 4 (Prostate, Bladder), and Group 5 (Liver, Pancreatic, Gall Bladder). Patients with 0-4 markers positive are presumed to be cancer-free, while patients with 5 markers positive will undergo a second step. Presumptive positive samples are then assayed in the second step, testing 1 or 2 groups, using 64 markers per group, wherein on average 36 such markers would exhibit an average sensitivity of 50% for each specific type of cancer within that group, including using tissue-specific markers to validate the initial result and to identify tissue of origin. Results are scored as follows: 0-3 positive markers are considered cancer-free; 4 positive markers are advised to come back in 3-6 months for retesting; 5 positive markers are directed to go to imaging that matches the type(s) of cancer most likely to be the tissue of origin. For higher sensitivities, both the initial 96 markers in the first step, and the group-specific markers in the second step would have average sensitivity of 66% (FIG. 1D). The physician may then order targeted sequencing to further guide treatment decisions for the patient.

The present application is directed to a universal diagnostic approach that seeks to combine the best features of digital polymerase chain reaction (PCR), or quantitative polymerase chain reaction (qPCR), with bisulfite conversion, ligation detection reaction (LDR), and quantitative detection of multiple disease markers, e.g., cancer markers.

### Multiplexing, Avoiding False-Positives, and Carryover Protection

There is a technical challenge of distinguishing true signal generated from the desired disease-specific nucleic acid differences vs. false signal generated from normal nucleic acids present in the sample vs. false signal generated in the absence of the disease-specific nucleic acid differences (i.e. somatic mutations).

A number of solutions to these challenges are presented below, but they share some common themes.

The first theme is multiplexing. PCR works best when primer concentration is relatively high, from 50 nM to 500 nM, limiting multiplexing. Further, the more PCR primer pairs added, the chances of amplifying incorrect products or creating primer-dimers increase exponentially. In contrast, for LDR probes, low concentrations on the order of 4 nM to 20 nM are used, and probe-dimers are limited by the requirement for adjacent hybridization on the target to allow for a ligation event. Use of low concentrations of gene-specific PCR primers or LDR probes containing universal primer sequence “tails” allows for subsequent addition of higher concentrations of universal primers to achieve proportional amplification of the initial PCR or LDR products. Another way to avoid or minimize false PCR amplicons or primer dimers is to use PCR primers containing a few extra bases and a blocking group, which is liberated to form a free 3′OH by cleavage with a nuclease only when hybridized to the target, e.g., a ribonucleotide base as the blocking group and RNase H2 as the cleaving nuclease.

The second theme is fluctuations in signal due to low input target nucleic acids. Often, the target nucleic acid originated from a few cells, either captured as CTCs, or from tumor cells that underwent apoptosis and released their DNA as small fragments (140-160 bp) in the serum. Under such conditions, it is preferable to perform some level of proportional amplification to avoid missing the signal altogether or reporting inaccurate copy number due to fluctuations when distributing small numbers of starting molecules into individual wells (for real-time, or droplet PCR quantification). As long as these initial amplifications are kept at a reasonable level (approximately 12 to 20 cycles), the risk of carryover contamination during opening of the tube and distributing amplicons for subsequent detection/quantification (using real-time, or droplet PCR) is minimized. Other schemes use even lower amounts of limited amplifications, (approximately 8 to 12 cycles).

The third theme is target-independent signal, also known as “No Template Control” (NTC). This arises from either polymerase or ligase reactions that occur in the absence of the correct target. Some of this signal may be minimized by judicious primer design. For ligation reactions, the 5′→3′ nuclease activity of polymerase may be used to liberate the 5′ phosphate of the downstream ligation primer (only when hybridized to the target), so it is suitable for ligation. Further specificity for distinguishing presence of a low-level mutation using LDR may be achieved by: (i) using upstream mutation-specific LDR probes containing a mismatch in the 2^{nd} or 3^{rd} position from the 3′OH base, (ii) using LNA or PNA probes to wild-type sequence that would reduce hybridization of mutation-specific LDR probes to wild-type sequences, (iii) using LDR probes to wild-type sequence that (optionally) ligate but do not undergo additional amplification, and (iv) using upstream LDR probes containing a few extra bases and a blocking group, which is liberated to form a free 3′OH by cleavage with a nuclease only when hybridized to the complementary target (e.g., RNase H2 and a ribonucleotide base). Similar approaches for improving the specificity for distinguishing presence of a low-level mutation using PCR may be achieved by: (i) using mutation-specific PCR Primers containing a mismatch in the 2^{nd} or 3^{rd} position from the 3′OH base, (ii) using LNA or PNA probes to wild-type sequence that would reduce hybridization of mutation-specific PCR primers to wild-type sequences, (iii) using PCR primers to wild-type sequence that are blocked and do not undergo additional amplification, and (iv) using upstream PCR primers containing a few extra bases and a blocking group, which is liberated to form a free 3′OH by cleavage with a nuclease only when hybridized to the complementary target (e.g., RNase H2 and a ribonucleotide base).

The fourth theme is either suppressed (reduced) amplification or incorrect (false) amplification due to unused primers in the reaction. One approach to eliminate such unused primers is to capture genomic or target or amplified target DNA on a solid support, allow ligation probes to hybridize and ligate, and then remove probes or products that are not hybridized. Alternative solutions include pre-amplification, followed by subsequent nested LDR and/or PCR steps, such that there is a second level of selection in the process.

The fifth theme is carryover prevention. Carryover signal may be eliminated by standard uracil incorporation during the universal PCR amplification step, and by using UDG (and optionally AP endonuclease) in the pre-amplification workup procedure. Incorporation of carryover prevention is central to the methods of the present application as described in more detail below. The initial PCR amplification is performed using incorporation of uracil. The LDR reaction is performed with LDR probes lacking uracil. Thus, when the LDR products are subjected to real-time PCR quantification, addition of UDG destroys the initial PCR products, but not the LDR products. Further, since LDR is a linear process and the tag primers use sequences absent from the human genome, accidental carryover of LDR products back to the original PCR will not cause template-independent amplification. Additional schemes to provide carryover prevention with methylated targets include use of restriction endonucleases to destroy unmethylated DNA prior to PCTR amplification, or capturing and enriching methylated DNA using methyl-specific DNA binding proteins or antibodies.

The sixth theme is achieving even amplification of many mutation-specific or methylation-specific targets in the multiplexed reaction. One approach, as already described above, is to perform limited initial PCR amplifications (8 to 12, or 12 to 20 cycles). However, sometimes different products amplify at different rates, especially when using mutation- or methylation-specific primers, or when using blocking LNA or PNA probes or other means to suppress amplification of wild-type DNA. This is because a regular PCR reaction has both forward and reverse primers working simultaneously. Although there may be preferential amplification using as an example a forward methylation-specific primer (i.e. after bisulfite treatment), the reverse primer will amplify both methylated and un-methylated DNA (again, after bisulfite treatment), and thus will magnify differences in initial rates of forward primer amplification. Further, and this also holds when using mutation-specific forward primers, the use of non-selecting reverse primers means that initial amplification products still contain substantial amounts of wild-type DNA sequence, which may lead to undesired false-positives in subsequent amplification steps. One approach is to perform an initial single-sided linear amplification, using primers that amplify only one strand of target DNA. This is particularly useful when amplifying bisulfite-treated DNA, where the two resultant strands are no longer complementary to each other. An important variation of this theme destroys the initial target DNA after the linear amplification step. This may be achieved by incorporating one or more modified nucleotides, such as α-thio-dNTPs, that protect the initial extension products (but not the original cfDNA or genomic DNA) from exonuclease I digestion. When using bisulfite converted DNA, after performing the initial single-sided linear amplification (i.e., the polymerase extension reaction) with regular dNTP's (i.e. NO dUTP), the original bisulfite-converted DNA may be destroyed using UDG.

### Methods of Identifying Cancer Markers

A first aspect of the present application is directed to a method for identifying, in a sample, one or more parent nucleic acid molecules containing a target nucleotide sequence differing from nucleotide sequences of other parent nucleic acid molecules in the sample by one or more nucleotides, one or more copy numbers, one or more transcript sequences, and/or one or more methylated residues. The method involves providing a sample containing one or more parent nucleic acid molecules potentially containing the target nucleotide sequence differing from the nucleotide sequences of other parent nucleic acid molecules by one or more nucleotides, one or more copy numbers, one or more transcript sequences, and/or one or more methylated residues. One or more enzymes capable of digesting deoxyuracil (dU)-containing nucleic acid molecules are then provided. One or more primary oligonucleotide primer sets are also provided. Each primary oligonucleotide primer set comprises (a) a first primary oligonucleotide primer that comprises a nucleotide sequence that is complementary to a sequence in the parent nucleic acid molecule adjacent to the target nucleotide sequence and (b) a second primary oligonucleotide primer that comprises a nucleotide sequence that is complementary to a portion of an extension product formed from the first primary oligonucleotide primer. The sample, the one or more first primary oligonucleotide primers of the primary oligonucleotide primer sets, the one or more enzymes capable of digesting deoxyuracil (dU)-containing nucleic acid molecules, a deoxynucleotide mix, and a DNA polymerase are blended to form one or more polymerase extension reaction mixtures, and the one or more polymerase extension reaction mixtures are subjected to conditions suitable for digesting deoxyuracil (dU)-containing nucleic acid molecules present in the polymerase extension reaction mixtures and for carrying out one or more polymerase extension reaction cycles comprising a denaturation treatment, a hybridization treatment, and an extension treatment, thereby forming primary extension products comprising nucleotide sequences complementary to the target nucleotide sequence. The one or more polymerase extension reaction mixtures comprising the primary extension products, the one or more second primary oligonucleotide primers of the primary oligonucleotide primer sets, the one or more enzymes capable of digesting deoxyuracil (dU)-containing nucleic acid molecules in the reaction mixtures, a deoxynucleotide mix including dUTP, and a DNA polymerase are blended to form one or more first polymerase chain reaction mixtures. The method further comprises subjecting the one or more first polymerase chain reaction mixtures to conditions suitable for digesting deoxyuracil (dU)-containing nucleic acid molecules present in the polymerase chain reaction mixtures and for carrying out one or more polymerase chain reaction cycles comprising a denaturation treatment, a hybridization treatment, and an extension treatment, thereby forming one or more first polymerase chain reaction products comprising the target nucleotide sequence or a complement thereof. One or more oligonucleotide probe sets are then provided. Each probe set comprises (a) a first oligonucleotide probe having a 5′ primer-specific portion and a 3′ target sequence-specific portion, and (b) a second oligonucleotide probe having a 5′ target sequence-specific portion and a 3′ primer-specific portion, wherein the first and second oligonucleotide probes of a probe set are configured to hybridize, in a base specific manner, on a complementary target nucleotide sequence of a secondary extension product. The one or more first polymerase chain reaction products are blended with a ligase, and the one or more oligonucleotide probe sets to form one or more ligation reaction mixtures. The one or more ligation reaction mixtures are subjected to one or more ligation reaction cycles whereby the first and second oligonucleotide probes of the one or more oligonucleotide probe sets are ligated together, when hybridized to complementary sequences, to form ligated product sequences in the ligation reaction mixtures wherein each ligated product sequence comprises the 5′ primer-specific portion, the target-specific portions, and the 3′ primer-specific portion. The method further includes providing one or more secondary oligonucleotide primer sets. Each secondary oligonucleotide primer set comprises (a) a first secondary oligonucleotide primer comprising the same nucleotide sequence as the 5′ primer-specific portion of the ligated product sequence and (b) a second secondary oligonucleotide primer comprising a nucleotide sequence that is complementary to the 3′ primer-specific portion of the ligated product sequence. The ligated product sequences, the one or more secondary oligonucleotide primer sets, the one or more enzymes capable of digesting deoxyuracil (dU)-containing nucleic acid molecules, a deoxynucleotide mix including dUTP, and a DNA polymerase are blended to form one or more second polymerase chain reaction mixtures. The one or more second polymerase chain reaction mixtures are subjected to conditions suitable for digesting deoxyuracil (dU)-containing nucleic acid molecules present in the second polymerase chain reaction mixtures and for carrying out one or more polymerase chain reaction cycles comprising a denaturation treatment, a hybridization treatment, and an extension treatment thereby forming one or more second polymerase chain reaction products. The method further comprises detecting and distinguishing the one or more second polymerase chain reaction products in the one or more second polymerase chain reaction mixtures to identify the presence of one or more parent nucleic acid molecules containing target nucleotide sequences differing from nucleotide sequences in other parent nucleic acid molecules in the sample by one or more nucleotides, one or more copy numbers, one or more transcript sequences, and/or one or more methylated residues.

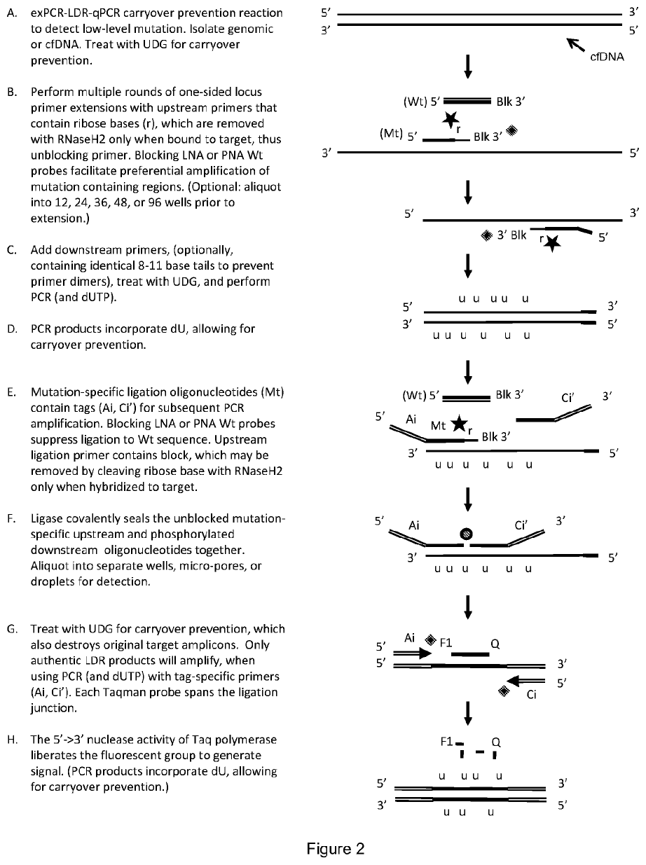

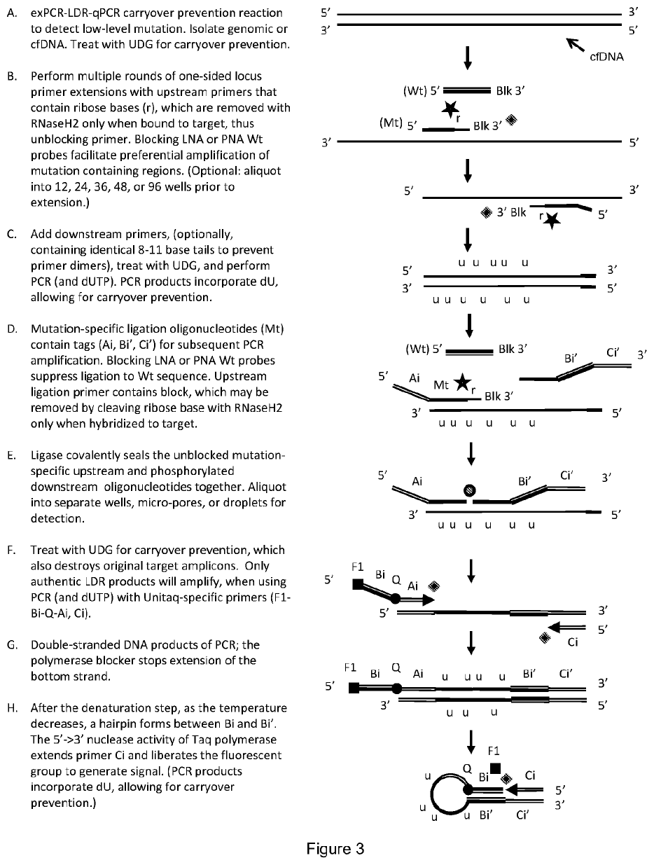

FIGS. 2 and 3 illustrate various embodiments of this aspect of the present application, abbreviated as exPCR-LDR-qPCR carryover prevention reaction to detect low-level mutations (exPCR is an abbreviation for one-sided extension using primers to one strand of a locus, followed by PCR—using either the same primers in the initial extension, or additional primers for the PCR step). Genomic or cfDNA is isolated (FIG. 2, step A), and the isolated DNA sample is treated with UDG to digest dU containing nucleic acid molecules that may be present in the sample (FIG. 2, step B). Suitable enzymes include, without limitation, E. coli uracil DNA glycosylase (UDG), Antarctic Thermolabile UDG, or Human single-strand-selective monofunctional uracil-DNA Glycosylase (hSMUG1). The regions of interest are selectively extended using locus-specific upstream primers, a blocking LNA or PNA probe comprising wild-type sequence, and a deoxynucleotide mix that includes dUTP. In this embodiment, another layer of selectivity can be incorporated into the method by including a 3′ cleavable blocking group (Blk 3′, e.g. C3 spacer), and an RNA base (r), in the upstream primer. Upon target-specific hybridization, RNase H (star symbol) removes the RNA base to liberate a 3′OH group which is a few bases upstream of the mutation, and suitable for polymerase extension (FIG. 2 or 3, step B; see e.g., Dobosy et. al. “RNase H-Dependent PCR (rhPCR): Improved Specificity and Single Nucleotide Polymorphism Detection Using Blocked Cleavable Primers,” BMC Biotechnology 11(80):1011 (2011), which is hereby incorporated by reference in its entirety). A blocking LNA or PNA probe comprising wild-type sequence that partially overlaps with the upstream PCR primer will preferentially compete in binding to wild-type sequence over the upstream primer, but not as much to mutant DNA, and thus suppresses extension of wild-type DNA during each round of primer extension. Sample is optionally aliquoted into 12, 24, 36, 48, or 96 wells prior to the initial extension step. Subsequently, the locus-specific downstream primers are added, followed by limited (8 to 20 cycles) or full (20-40 cycles) PCR. Optionally, the downstream primers contain identical 8-11 base tails to prevent primer dimers. Further, such tails provide the option for asymmetric PCR at the end of the PCR cycles, by raising the hybridization temperature above that for the forward primers, but at or below that for the reverse primers—which at 8-11 bases longer will have higher Tm values. This generates more bottom strand products, which are suitable substrates for the subsequent LDR step. In an alternative embodiment, the initial extension products incorporate one or more modified nucleotides, such as α-thio-dNTPs, that protect the initial extension products (but not the original cfDNA or genomic DNA) from exonuclease I digestion. After exonuclease I digestion, the down-stream locus-specific primers (optionally containing identical 8-11 base tails) are added, again followed by limited (8 to 20 cycles) or full (20-40 cycles) PCR. The amplified products contain dU as shown in FIG. 2 or 3, step D, which allows for subsequent treatment with UDG or a similar enzyme for carryover prevention.

As shown in FIG. 2 step E, target-specific oligonucleotide probes are hybridized to the amplified products and ligase (filled circle) covalently seals the two oligonucleotides together when hybridized to their complementary sequence. In this embodiment, the upstream oligonucleotide probe having a sequence specific for detecting the mutation of interest further contains a 5′ primer-specific portion (Ai) to facilitate subsequent detection of the ligation product. Once again, the presence of blocking LNA or PNA probe comprising wild-type sequence suppresses ligation to wild-type target sequence if present after the enrichment of mutant sequence during the PCR amplification step. The downstream oligonucleotide probe, having a sequence common to both mutant and wild-type sequences contains a 3′ primer-specific portion (Ci′) that, together with the 5′ primer specific portion (Ai) of the upstream probe having a sequence specific for detecting the mutation, permit subsequent amplification and detection of only mutant ligation products. As illustrated in step E of FIG. 2, another layer of specificity can be incorporated into the method by including a 3′ cleavable blocking group (Blk 3′, e.g. C3 spacer), and an RNA base (r), in the upstream ligation probe. Upon target-specific hybridization, RNase H (star symbol) removes the RNA base to generate a ligation competent 3′OH group (FIG. 2, step D).

As shown in FIG. 2, step F, target-specific oligonucleotide probes are hybridized to the amplified products and ligase (filled circle) covalently seals the two oligonucleotides together when hybridized to their complementary sequence. The upstream oligonucleotide probe contains a 5′ primer-specific portion (Ai) and the downstream oligonucleotide probe contains a 3′ primer-specific portion (Ci′) that permits subsequent amplification of the ligation product. Following ligation, the ligation products are aliquoted into separate wells, micro-pores or droplets containing one or more tag-specific primer pairs, each pair comprising matched primers Ai and Ci, treated with UDG or similar enzyme to remove dU containing amplification products or contaminants, PCR amplified, and detected. As shown in FIG. 2, steps G & H, detection of the ligation product can be carried out using traditional TaqMan™ detection assay (see U.S. Pat. No. 6,270,967 to Whitcombe et al., and U.S. Pat. No. 7,601,821 to Anderson et al., which are hereby incorporated by reference in their entirety). For detection using TaqMan™ an oligonucleotide probe spanning the ligation junction is used in conjunction with primers suitable for hybridization on the primer-specific portions of the ligation products for amplification and detection. The TaqMan™ probe contains a fluorescent reporter group on one end (F1) and a quencher molecule (Q) on the other end that are in close enough proximity to each other in the intact probe that the quencher molecule quenches fluorescence of the reporter group. During amplification, the TaqMan™ probe and upstream primer hybridize to their complementary regions of the ligation product. The 5′→3′ nuclease activity of the polymerase extends the hybridized primer and liberates the fluorescent group of the TaqMan™ probe to generate a detectable signal (FIG. 2, step H). In a preferred embodiment, the Taqman probe contains a second quencher group (ZEN) about 9 bases in from the fluorescent reporter group, and the probe is designed such that the ZEN group is at or adjacent to the mutant base. Use of dUTP during the amplification reaction generates products containing dU, which can subsequently be destroyed using UDG for carryover prevention.

As shown in FIG. 3 step D, target-specific oligonucleotide probes are hybridized to the amplified products and ligase (filled circle) covalently seals the two oligonucleotides together when hybridized to their complementary sequence. In this embodiment, the upstream oligonucleotide probe having a sequence specific for detecting the mutation of interest further contains a 5′ primer-specific portion (Ai) to facilitate subsequent detection of the ligation product. Once again, the presence of blocking LNA or PNA probe comprising wild-type sequence suppresses ligation to wild-type target sequence if present after the enrichment of mutant sequence during the PCR amplification step. The downstream oligonucleotide probe, having a sequence common to both mutant and wild-type sequences contains a 3′ primer-specific portion (Bi-Ci′) that, together with the 5′ primer specific portion (Ai) of the upstream probe having a sequence specific for detecting the mutation, permit subsequent amplification and detection of only mutant ligation products. As illustrated in step D of FIG. 3, another layer of specificity can be incorporated into the method by including a 3′ cleavable blocking group (Blk 3′, e.g. C3 spacer), and an RNA base (r), in the upstream ligation probe. Upon target-specific hybridization, RNase H (star symbol) removes the RNA base to generate a ligation competent 3′OH group (FIG. 3, step D).

In this embodiment, the ligation probes are designed to contain UniTaq primer and tag sequences to facilitate detections. The UniTaq system is fully described in U.S. Patent Application Publication No. 2011/0212846 to Spier, which is hereby incorporated by reference in its entirety. The UniTaq system involves the use of three unique “tag” sequences, where at least one of the unique tag sequences (Ai) is present in the first oligonucleotide probe, and the second and third unique tag portions (Bi′ and Ci′) are in the second oligonucleotide probe sequence as shown in FIG. 3, step D & E. Upon ligation of oligonucleotide probes in a probe set, the resulting ligation product will contain the Ai sequence—target specific sequences—Bi′ sequence—Ci′ sequence. The essence of the UniTaq approach is that both oligonucleotide probes of a ligation probe set need to be correct in order to get a positive signal, which allows for highly multiplexed nucleic acid detection. For example, and as described herein, this is achieved by requiring hybridization of two parts, i.e., two of the tags, to each other.

Prior to detecting the ligation product, the sample is treated with UDG to destroy original target amplicons allowing only authentic ligation products to be detected. Following ligation, the ligation products are aliquoted into separate wells, micro-pores or droplets containing one or more tag-specific primer pairs. For the detection step, the ligation product containing Ai (a first primer-specific portion), Bi′ (a UniTaq detection portion), and Ci′ (a second primer-specific portion) is primed on both strands using a first oligonucleotide primer having the same nucleotide sequence as Ai, and a second oligonucleotide primer that is complementary to Ci′ (i.e., Ci). The first oligonucleotide primer also includes a UniTaq detection probe (Bi) that has a detectable label F1 on one end and a quencher molecule (Q) on the other end (F1-Bi-Q-Ai). Optionally positioned proximal to the quencher is a polymerase-blocking unit, e.g., HEG, THF, Sp-18, ZEN, or any other blocker known in the art that is sufficient to stop polymerase extension. In another embodiment, a ZEN quencher group is also positioned about 9 bases from the fluorescent reporter group to assure more complete quenching. PCR amplification results in the formation of double stranded products as shown in FIG. 3, step G). In this example, a polymerase-blocking unit prevents a polymerase from copying the 5′ portion (Bi) of the first universal primer, such that the bottom strand of product cannot form a hairpin when it becomes single-stranded. Formation of such a hairpin would result in the 3′ end of the stem annealing to the amplicon such that polymerase extension of this 3′ end would terminate the PCR reaction.

The double stranded PCR products are denatured, and when the temperature is subsequently decreased, the upper strand of product forms a hairpin having a stem between the 5′ portion (Bi) of the first oligonucleotide primer and portion Bi′ at the opposite end of the strand (FIG. 3, step H). Also, during this step, the second oligonucleotide primer anneals to the 5′-primer specific portion (Ci′) of the hairpinned product. Upon extension of the second universal primer in step H, 5′ nuclease activity of the polymerase cleaves the detectable label D1 or the quencher molecule from the 5′ end of the amplicon, thereby increasing the distance between the label and the quencher and permitting detection of the label.

The ligation reaction used in the methods of the present application is well known in the art. Ligases suitable for ligating oligonucleotide probes of a probe set together (optionally following cleavage of a 3′ ribose and blocking group on the first oligonucleotide probe, or the 5′ flap on the second oligonucleotide probe) include, without limitation Thermus aquaticus ligase, E. coli ligase, T4 DNA ligase, T4 RNA ligase, Tag ligase, 9 N ligase, and Pyrococcus ligase, or any other thermostable ligase known in the art. In accordance with the present application, the nuclease-ligation process of the present application can be carried out by employing an oligonucleotide ligation assay (OLA) reaction (see Landegren, et al., “A Ligase-Mediated Gene Detection Technique,” Science 241:1077-80 (1988); Landegren, et al., “DNA Diagnostics—Molecular Techniques and Automation,” Science 242:229-37 (1988); and U.S. Pat. No. 4,988,617 to Landegren, et al., which are hereby incorporated by reference in their entirety), a ligation detection reaction (LDR) that utilizes one set of complementary oligonucleotide probes (see e.g., WO 90/17239 to Barany et al, which is hereby incorporated by reference in its entirety), or a ligation chain reaction (LCR) that utilizes two sets of complementary oligonucleotide probes see e.g., WO 90/17239 to Barany et al, which is hereby incorporated by reference in its entirety).

The oligonucleotide probes of a probe sets can be in the form of ribonucleotides, deoxynucleotides, modified ribonucleotides, modified deoxyribonucleotides, peptide nucleotide analogues, modified peptide nucleotide analogues, modified phosphate-sugar-backbone oligonucleotides, nucleotide analogs, and mixtures thereof.

The hybridization step in the ligase detection reaction, which is preferably a thermal hybridization treatment, discriminates between nucleotide sequences based on a distinguishing nucleotide at the ligation junctions. The difference between the target nucleotide sequences can be, for example, a single nucleic acid base difference, a nucleic acid deletion, a nucleic acid insertion, or rearrangement. Such sequence differences involving more than one base can also be detected. Preferably, the oligonucleotide probe sets have substantially the same length so that they hybridize to target nucleotide sequences at substantially similar hybridization conditions.

Ligase discrimination can be further enhanced by employing various probe design features. For example, an intentional mismatch or nucleotide analogue (e.g., Inosine, Nitroindole, or Nitropyrrole) can be incorporated into the first oligonucleotide probe at the 2^{nd} or 3^{rd} base from the 3′ junction end to slightly destabilize hybridization of the 3′ end if it is perfectly matched at the 3′ end, but significantly destabilize hybridization of the 3′ end if it is mis-matched at the 3′ end. This design reduces inappropriate misligations when mutant probes hybridize to wild-type target. Alternatively, RNA bases that are cleaved by RNases can be incorporated into the oligonucleotide probes to ensure template-dependent product formation. For example, Dobosy et. al. “RNase H-Dependent PCR (rhPCR): Improved Specificity and Single Nucleotide Polymorphism Detection Using Blocked Cleavable Primers,” BMC Biotechnology 11(80): 1011 (2011), which is hereby incorporated by reference in its entirety, describes using an RNA-base close to the 3′ end of an oligonucleotide probe with 3′-blocked end, and cutting it with RNase H2 generating a PCR-extendable and ligatable 3′-OH. This approach can be used to generate either ligation-competent 3′OH (for standard DNA ligases) or 5′-P, or both, in the latter case, provided a ligase that can ligate 5′-RNA base is utilized.

Other possible modifications included abasic sites, e.g., internal abasic furan or oxo-G. These abnormal “bases” are removed by specific enzymes to generate ligation-competent 3′-OH or 5′P sites. Endonuclease IV, Tth EndoIV (NEB) will remove abasic residues after the ligation oligonucleotides anneal to the target nucleic acid, but not from a single-stranded DNA. Similarly, one can use oxo-G with Fpg or inosine/uracil with EndoV or Thymine glycol with EndoVIII.

Ligation discrimination can also be enhanced by using the coupled nuclease-ligase reaction described in WO2013/123220 to Barany et al. or U.S. Patent Application Publication No. 2006/0234252 to Anderson et al., which are hereby incorporated by reference in their entirety. In this embodiment, the first oligonucleotide probe bears a ligation competent 3′ OH group while the second oligonucleotide probe bears a ligation incompetent 5′ end (i.e., an oligonucleotide probe without a 5′ phosphate). The oligonucleotide probes of a probe set are designed such that the 3′-most base of the first oligonucleotide probe is overlapped by the immediately flanking 5′-most base of the second oligonucleotide probe that is complementary to the target nucleic acid molecule. The overlapping nucleotide is referred to as a “flap”. When the overlapping flap nucleotide of the second oligonucleotide probe is complementary to the target nucleic acid molecule sequence and the same sequence as the terminating 3′ nucleotide of the first oligonucleotide probe, the phosphodiester bond immediately upstream of the flap nucleotide of the second oligonucleotide probe is discriminatingly cleaved by an enzyme having flap endonuclease (FEN) or 5′ nuclease activity. That specific FEN activity produces a novel ligation competent 5′ phosphate end on the second oligonucleotide probe that is precisely positioned alongside the adjacent 3′ OH of the first oligonucleotide probe to allow ligation of the two probes to occur. In accordance with this embodiment, flap endonucleases or 5′ nucleases that are suitable for cleaving the 5′ flap of the second oligonucleotide probe prior to ligation include, without limitation, polymerases with 5′ nuclease activity such as E. coli DNA polymerase and polymerases from Taq and T. thermophilus, as well as T4 RNase H and TaqExo. In another embodiment, the second probe of the probe set has a 3′ primer-specific portion, a target specific portion, and a 5′ nucleotide sequence, where the 5′ nucleotide sequence is complementary to at least a portion of the 3′ primer-specific portion, and where the 5′ nucleotide sequence hybridizes to its complementary portion of the 3′ primer-specific portion to form a hair-pinned second oligonucleotide probe when the second probe is not hybridized to a target nucleotide sequence.

For insertions or deletions, incorporation of a matched base or nucleotide analogues (e.g., -amino-dA or 5-propynyl-dC) in the first oligonucleotide probe at the 2^{nd} or 3^{rd} position from the junction improves stability and may improve discrimination of such frameshift mutations from wild-type sequences. For insertions, use of one or more thiophosphate-modified nucleotides downstream from the desired scissile phosphate bond of the second oligonucleotide probe will prevent inappropriate cleavage by the 5′ nuclease enzyme when the probes are hybridized to wild-type DNA, and thus reduce false-positive ligation on wild-type target. Likewise, for deletions, use of one or more thiophosphate-modified nucleotides upstream from the desired scissile phosphate bond of the second oligonucleotide probe will prevent inappropriate cleavage by the 5′ nuclease enzyme when the probes are hybridized to wild-type DNA, and thus reduce false-positive ligation on wild-type target.

Another aspect of the present application is directed to a method for identifying, in a sample, one or more parent nucleic acid molecules containing a target nucleotide sequence differing from nucleotide sequences in other parent nucleic acid molecules in the sample by one or more nucleotides, one or more copy numbers, one or more transcript sequences, and/or one or more methylated residues. The method involves providing a sample containing one or more parent nucleic acid molecules potentially containing the target nucleotide sequence differing from the nucleotide sequences of other parent nucleic acid molecules by one or more nucleotides, one or more copy numbers, one or more transcript sequences, and/or one or more methylated residues. One or more enzymes capable of digesting deoxyuracil (dU) containing nucleic acid molecules, one or more nucleases capable of digesting nucleic acid molecules not comprising modified nucleotides, and one or more first primary oligonucleotide primer(s) are provided. The one or more first primary oligonucleotide primer(s) comprise a nucleotide sequence that is complementary to a sequence in the parent nucleic acid molecule adjacent to the target nucleotide sequence. The sample, the one or more first primary oligonucleotide primers, the one or more enzymes capable of digesting deoxyuracil (dU)-containing nucleic acid molecules, a deoxynucleotide mix that comprises one or more modified nucleotides that protect extension products but not target DNA from nuclease digestion, and a DNA polymerase are blended to form one or more polymerase extension reaction mixtures, and the one or more polymerase extension reaction mixtures are subjected to conditions suitable for digesting deoxyuracil (dU)-containing nucleic acid molecules present in the polymerase extension reaction mixture and for carrying out one or more polymerase extension reaction cycles comprising a denaturation treatment, a hybridization treatment, and an extension treatment, thereby forming primary extension products comprising the complement of the target nucleotide sequence. The method further comprises providing one or more secondary oligonucleotide primer sets. Each secondary oligonucleotide primer set comprises (a) a first secondary oligonucleotide primer having a first 5′ primer-specific portion and a 3′ portion that is complementary to a portion of a primary extension product formed from the first primary oligonucleotide primer and (b) a second secondary oligonucleotide primer having a second 5′ primer-specific portion and a 3′ portion that comprises a nucleotide sequence that is complementary to a portion of an extension product formed from the first secondary oligonucleotide primer. The one or more polymerase extension reaction mixtures comprising the primary extension products, the one or more secondary oligonucleotide primer sets, the one or more nucleases, a deoxynucleotide mix, and a DNA polymerase are blended to form one or more first polymerase chain reaction mixtures, and the one or more first polymerase chain reaction mixtures are subjected to conditions suitable for digesting nucleic acid molecules present in the first polymerase chain reaction mixtures, but not primary extension products comprising modified nucleotides and for carrying out two or more polymerase chain reaction cycles comprising a denaturation treatment, a hybridization treatment, and an extension treatment, thereby forming one or more first polymerase chain reaction products comprising the first 5′ primer-specific portion, a target-specific nucleotide sequence or a complement thereof, and a complement of the second 5′ primer-specific portion. One or more tertiary oligonucleotide primer sets are provided. Each tertiary oligonucleotide primer set comprises (a) a first tertiary oligonucleotide primer comprising the same nucleotide sequence as the first 5′ primer-specific portion of the one or more first polymerase chain reaction products and (b) a second tertiary oligonucleotide primer comprising a nucleotide sequence that is complementary to the 3′ primer-specific portion of the one or more first polymerase chain reaction products. The one or more first polymerase chain reaction products, the one or more tertiary oligonucleotide primer sets, the one or more enzymes capable of digesting deoxyuracil (dU) containing nucleic acid molecules, a deoxynucleotide mix including dUTP, and a DNA polymerase are blended to form one or more second polymerase chain reaction mixtures, and the one or more second polymerase chain reaction mixtures are subjected to conditions suitable for digesting deoxyuracil (dU) containing nucleic acid molecules present in the second polymerase chain reaction mixtures and for carrying out one or more polymerase chain reaction cycles comprising a denaturation treatment, a hybridization treatment, and an extension treatment, thereby forming one or more second polymerase chain reaction products. The method further involves detecting and distinguishing the one or more second polymerase chain reaction products in the one or more second polymerase chain reaction mixtures to identify the presence of one or more parent nucleic acid molecules containing target nucleotide sequences differing from nucleotide sequences in other parent nucleic acid molecules in the sample by one or more nucleotides, one or more copy numbers, one or more transcript sequences, and/or one or more methylated residues.

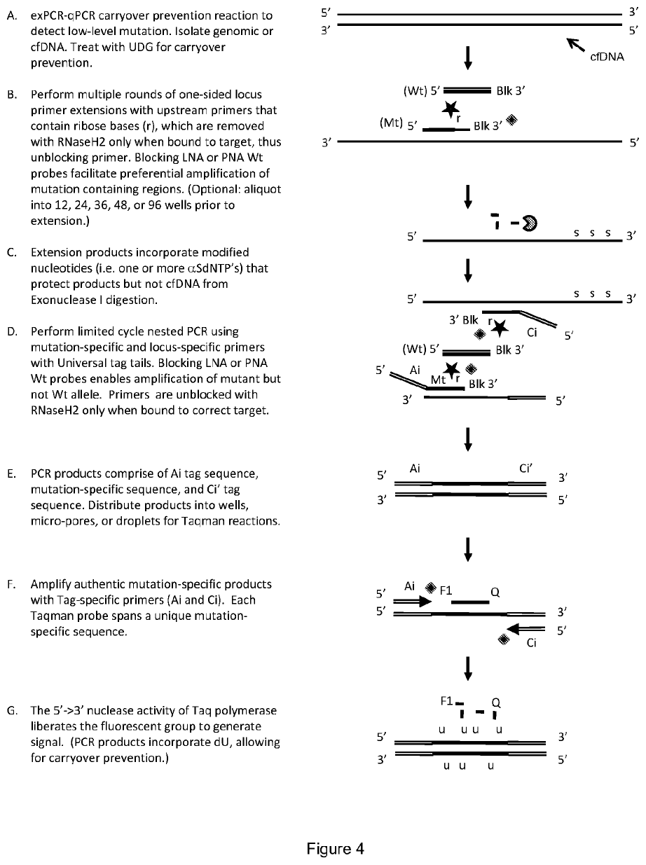

FIGS. 4-8 illustrate various embodiments of this aspect of the present application.

FIG. 4 illustrates an exemplary exPCR-qPCR carryover prevention reaction to detect low-level mutations. Genomic or cfDNA is isolated (FIG. 4, step A), and the isolated DNA sample is treated with UDG to digest dU containing nucleic acid molecules that may be present in the sample (FIG. 4, step A). The sample is then subject to a linear amplification reaction, e.g., one or more polymerase extension reactions to generate complementary copies of mutation containing regions of interest. The regions of interest are selectively extended using locus-specific upstream primers, a blocking LNA or PNA probe comprising wild-type sequence, and a deoxynucleotide mix that includes one of more modified nucleotides. In this embodiment, another layer of selectivity can be incorporated into the method by including a 3′ cleavable blocking group (Blk 3′, e.g. C3 spacer), and an RNA base (r), in the upstream primer. Upon target-specific hybridization, RNase H (star symbol) removes the RNA base to liberate a 3′OH group which is a few bases upstream of the mutation, and suitable for polymerase extension (FIG. 4, step B). A blocking LNA or PNA probe comprising wild-type sequence that partially overlaps with the upstream PCR primer will preferentially compete in binding to wild-type sequence over the upstream primer, but not as much to mutant DNA, and thus suppresses extension of wild-type DNA during each round of primer extension. Optionally aliquot sample into 12, 24, 36, 48, or 96 wells prior to the initial extension step.

The initial extension products incorporate one or more modified nucleotides, such as α-thio-dNTPs, that protect the initial extension products (but not the original cfDNA or genomic DNA) from exonuclease I digestion (FIG. 4, step C). Using just upstream locus-specific primers in the presence of blocking LNA or PNA probes enriches for extension of mutation-containing products with each extension cycle. The exonuclease digestion destroys wild-type DNA present in the original genomic or cfDNA sample, and thus the enriched extension products will not be diluted by subsequent extension or amplification off original wild-type DNA (see step D below).

As shown in FIG. 4 step D, mutation-specific and locus-specific oligonucleotide primers are added to then perform limited cycle nested PCR to amplify the mutation-containing sequence, if present in the sample. In this embodiment, the upstream mutation-specific primer having a sequence specific for detecting the mutation of interest further contains a 5′ primer-specific portion (Ai) to facilitate subsequent detection of the nested PCR product. Once again, the presence of blocking LNA or PNA probe comprising wild-type sequence suppresses extension of wild-type target sequence if present after the enrichment of mutant sequence during the initial extension step. The reverse locus-specific primer, having a sequence common to both mutant and wild-type sequences contains a 5′ primer-specific portion (Ci) that, together with the 5′ primer specific portion (Ai) of the upstream primer having a sequence specific for detecting the mutation, permit subsequent amplification and detection of only mutant PCR products. As illustrated in step D of FIG. 4, another layer of specificity can be incorporated into the method by including a 3′ cleavable blocking group (Blk 3′, e.g. C3 spacer), and an RNA base (r), in the mutation-specific and locus-specific primers. Upon target-specific hybridization, RNase H (star symbol) removes the RNA base to generate a polymerase extension competent 3′OH group (FIG. 4, step D). In the initial primer extension (step B) the liberated 3′OH base is a few bases upstream from the mutation position, and thus would extend both wild-type and mutant sequences if cleaved (although the blocking LNA or PNA should limit cleavage of primer hybridized to wild-type sequence). In contrast, in the nested PCR (step D), the mutation-specific base of the primer is at the 3′OH base, such that extension on wild-type sequence would be less likely, since the base is mismatched. The specificity for polymerase extension of mutant over wild-type sequence may be further improved by: (i) using mutation-specific PCR Primers containing a mismatch in the 2^{nd} or 3^{rd} position from the 3′OH base, (ii) using LNA or PNA probes to wild-type sequence that would reduce hybridization of mutation-specific PCR primers to wild-type sequences, (iii) using PCR primers to wild-type sequence that are blocked and do not undergo additional amplification, and (iv) avoiding G:T or T:G mismatches between primer and wild-type sequence at the 3′OH base.

As shown in FIG. 4 step E, nested PCR products comprise a 5′ primer-specific portion (Ai) target-specific sequence, and a 3′ primer-specific portion (Ci′) that permits subsequent amplification of the nested PCR product. Following the limited cycle PCR, the PCR products are aliquoted into separate wells, micro-pores or droplets containing one or more tag-specific primer pairs, each pair comprising matched primers Ai and Ci, treated with UDG or similar enzyme to remove dU containing amplification products or contaminants, PCR amplified, and detected. As shown in FIG. 4, steps F & G, detection of the ligation product can be carried out using traditional TaqMan™ detection assay (see U.S. Pat. No. 6,270,967 to Whitcombe et al., and U.S. Pat. No. 7,601,821 to Anderson et al., which are hereby incorporated by reference in their entirety). For detection using TaqMan™ an oligonucleotide probe spanning the mutation-specific region is used in conjunction with primers suitable for hybridization on the primer-specific portions of the nested PCR products for amplification and detection. The TaqMan™ probe contains a fluorescent reporter group on one end (F1) and a quencher molecule (Q) on the other end that are in close enough proximity to each other in the intact probe that the quencher molecule quenches fluorescence of the reporter group. During amplification, the TaqMan™ probe and upstream primer hybridize to their complementary regions of the nested PCR product. The 5′→3′ nuclease activity of the polymerase extends the hybridized primer and liberates the fluorescent group of the TaqMan™ probe to generate a detectable signal (FIG. 4, step G). In a preferred embodiment, the TaqMan™ probe contains a second quencher group (ZEN) about 9 bases in from the fluorescent reporter group, and the probe is designed such that the ZEN group is at or adjacent to the mutant base. Use of dUTP during the amplification reaction generates products containing dU, which can subsequently be destroyed using UDG for carryover prevention.

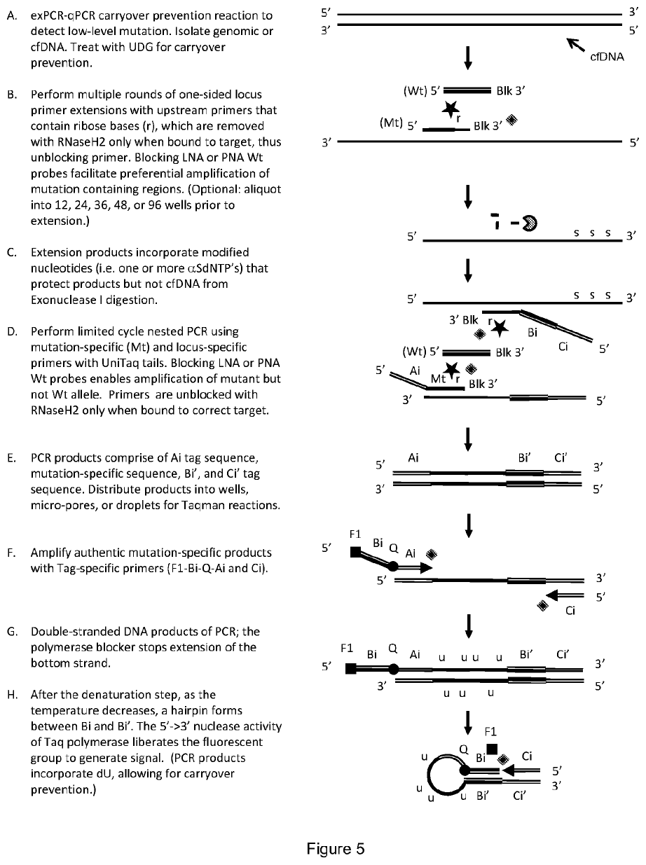

FIG. 5 illustrates an another exPCR-qPCR carryover prevention reaction to detect low-level mutations. Genomic or cfDNA is isolated (FIG. 5, step A), and the isolated DNA sample is treated with UDG to digest dU containing nucleic acid molecules that may be present in the sample (FIG. 5, step A). The sample is then subject to a linear amplification reaction, e.g., one or more polymerase extension reactions to generate complementary copies of mutation containing regions of interest. The regions of interest are selectively extended using locus-specific upstream primers, a blocking LNA or PNA probe comprising wild-type sequence, and a deoxynucleotide mix that includes one of more modified nucleotides. In this embodiment, another layer of selectivity can be incorporated into the method by including a 3′ cleavable blocking group (Blk 3′, e.g. C3 spacer), and an RNA base (r), in the upstream primer. Upon target-specific hybridization, RNase H (star symbol) removes the RNA base to liberate a 3′OH group which is a few bases upstream of the mutation, and suitable for polymerase extension (FIG. 5, step B). A blocking LNA or PNA probe comprising wild-type sequence that partially overlaps with the upstream PCR primer will preferentially compete in binding to wild-type sequence over the upstream primer, but not as much to mutant DNA, and thus suppresses extension of wild-type DNA during each round of primer extension. Optionally aliquot sample into 12, 24, 36, 48, or 96 wells prior to the initial extension step.

The initial extension products incorporate one or more modified nucleotides, such as α-thio-dNTPs, that protect the initial extension products (but not the original cfDNA or genomic DNA) from exonuclease I digestion (FIG. 5, step C). Using just upstream locus-specific primers in the presence of blocking LNA or PNA probes enriches for extension of mutation-containing products with each extension cycle. The exonuclease digestion destroys wild-type DNA present in the original genomic or cfDNA sample, and thus the enriched extension products will not be diluted by subsequent extension or amplification off original wild-type DNA (see step D below).

As shown in FIG. 5 step D, mutation-specific and locus-specific oligonucleotide primers are added to then perform limited cycle nested PCR to amplify the mutation-containing sequence, if present in the sample. In this embodiment, the upstream mutation-specific primer having a sequence specific for detecting the mutation of interest further contains a 5′ primer-specific portion (Ai) to facilitate subsequent detection of the nested PCR product. Once again, the presence of blocking LNA or PNA probe comprising wild-type sequence suppresses extension of wild-type target sequence if present after the enrichment of mutant sequence during the initial extension step. The reverse locus-specific primer, having a sequence common to both mutant and wild-type sequences contains a 3′ primer-specific portion (Bi-Ci) that, together with the 5′ primer specific portion (Ai) of the upstream primer having a sequence specific for detecting the mutation, permit subsequent amplification and detection of only mutant PCR products. As illustrated in step D of this Figure, another layer of specificity can be incorporated into the method by including a 3′ cleavable blocking group (Blk 3′, e.g. C3 spacer), and an RNA base (r), in the mutation-specific and locus-specific primers. Upon target-specific hybridization, RNase H (star symbol) removes the RNA base to generate a polymerase extension competent 3′OH group (FIG. 5, step D). In the initial primer extension (step B) the liberated 3′OH base is a few bases upstream from the mutation position, and thus would extend both wild-type and mutant sequences if cleaved (although the blocking LNA or PNA should limit cleavage of primer hybridized to wild-type sequence). In contrast, in the nested PCR (step D), the mutation-specific base of the primer is at the 3′OH base, such that extension on wild-type sequence would be less likely, since the base is mismatched. The specificity for polymerase extension of mutant over wild-type sequence may be further improved by: (i) using mutation-specific PCR Primers containing a mismatch in the 2^{nd} or 3^{rd} position from the 3′OH base, (ii) using LNA or PNA probes to wild-type sequence that would reduce hybridization of mutation-specific PCR primers to wild-type sequences, (iii) using PCR primers to wild-type sequence that are blocked and do not undergo additional amplification, and (iv) avoiding G:T or T:G mismatches between primer and wild-type sequence at the 3′OH base.

As shown in FIG. 5 step E, nested PCR products comprise a 5′ primer-specific portion (Ai) target-specific sequence, and a 3′ primer-specific portion (Bi′-Ci′) that permits subsequent amplification of the nested PCR product. Following the limited cycle PCR, the PCR products are aliquoted into separate wells, micro-pores or droplets containing one or more tag-specific primer pairs, each pair comprising matched primers F1-Bi-Q-Ai and Ci, treated with UDG or similar enzyme to remove dU containing amplification products or contaminants, PCR amplified (FIG. 5, step F), and detected. PCR amplification results in the formation of double stranded products as shown in FIG. 5, step G. In this example, a polymerase-blocking unit prevents a polymerase from copying the 5′ portion (Bi) of the first universal primer, such that the bottom strand of product cannot form a hairpin when it becomes single-stranded. Formation of such a hairpin would result in the 3′ end of the stem annealing to the amplicon such that polymerase extension of this 3′ end would terminate the PCR reaction.

The double stranded PCR products are denatured, and when the temperature is subsequently decreased, the upper strand of product forms a hairpin having a stem between the 5′ portion (Bi) of the first oligonucleotide primer and portion Bi′ at the opposite end of the strand (FIG. 5, step H). Also, during this step, the second oligonucleotide primer anneals to the 5′-primer specific portion (Ci′) of the hairpinned product. Upon extension of the second universal primer in step H, 5′ nuclease activity of the polymerase cleaves the detectable label D1 or the quencher molecule from the 5′ end of the amplicon, thereby increasing the distance between the label and the quencher and permitting detection of the label.

Another aspect of the present application is directed to a method for identifying, in a sample, one or more parent nucleic acid molecules containing a target nucleotide sequence differing from nucleotide sequences of other parent nucleic acid molecules in the sample by one or more nucleotides, one or more copy numbers, one or more transcript sequences, and/or one or more methylated residues. The method involves providing a sample containing one or more parent nucleic acid molecules potentially containing the target nucleotide sequence differing from the nucleotide sequences of other parent nucleic acid molecules by one or more nucleotides, one or more copy numbers, one or more transcript sequences, and/or one or more methylated residues. One or more enzymes capable of digesting deoxyuracil (dU)-containing nucleic acid molecules and one or more nucleases capable of digesting nucleic acid molecules present not comprising modified nucleotides are provided. The method also involves providing one or more primary oligonucleotide primer sets. Each primary oligonucleotide primer set comprises (a) a first primary oligonucleotide primer that comprises a nucleotide sequence that is complementary to a sequence in the parent nucleic acid molecule adjacent to the target nucleotide sequence and (b) a second primary oligonucleotide primer that comprises a nucleotide sequence that is complementary to a portion of an extension product formed from the first primary oligonucleotide primer. The sample, the one or more first primary oligonucleotide primers of the primary oligonucleotide primer sets, the one or more enzymes capable of digesting deoxyuracil (dU)-containing nucleic acid molecules, a deoxynucleotide mix that comprises one or more modified nucleotides that protect extension product but not target DNA from nuclease digestion, and a DNA polymerase are blended to form one or more polymerase extension reaction mixtures, and the one or more polymerase extension reaction mixtures are subjected to conditions suitable for digesting deoxyuracil (dU)-containing nucleic acid molecules present in the polymerase extension reaction mixtures and for carrying out one or more polymerase extension reaction cycles comprising a denaturation treatment, a hybridization treatment, and an extension treatment, thereby forming primary extension products comprising the complement of the target nucleotide sequence. The method further comprises blending the one or more polymerase extension reaction mixtures comprising the primary extension products, the one or more second primary oligonucleotide primers of the one or more primary oligonucleotide primer sets, the one or more nucleases, a deoxynucleotide mix, and a DNA polymerase to form one or more first polymerase chain reaction mixtures. The one or more first polymerase chain reaction mixtures are subjected to conditions suitable for digesting nucleic acid molecules present in the polymerase chain reaction mixtures, but not primary extension products comprising modified nucleotides and for carrying out two or more polymerase chain reaction cycles comprising a denaturation treatment, a hybridization treatment, and an extension treatment, thereby forming first polymerase chain reaction products comprising the target nucleotide sequence or a complement thereof. One or more secondary oligonucleotide primer sets are then provided. Each secondary oligonucleotide primer set comprises (a) a first secondary oligonucleotide primer having a 3′ portion that is complementary to a portion of an extension product formed from the first primary oligonucleotide primer and (b) a second secondary oligonucleotide primer having a 3′ portion that comprises a nucleotide sequence that is complementary to a portion of an extension product formed from the first secondary oligonucleotide primer. The first polymerase chain reaction products, the one or more secondary oligonucleotide primer sets, the one or more enzymes capable of digesting deoxyuracil (dU) containing nucleic acid molecules, a deoxynucleotide mix including dUTP, and a DNA polymerase are blended to form one or more second polymerase chain reaction mixtures. The one or more second polymerase chain reaction mixtures are subjected to conditions suitable for digesting deoxyuracil (dU)-containing nucleic acid molecules present in the second polymerase chain reaction mixtures and for carrying out two or more polymerase chain reaction cycles comprising a denaturation treatment, a hybridization treatment, and an extension treatment thereby forming second polymerase chain reaction products. The method further comprises detecting and distinguishing the second polymerase chain reaction products in the one or more second polymerase chain reaction mixtures to identify the presence of one or more parent nucleic acid molecules containing target nucleotide sequences differing from nucleotide sequences in other parent nucleic acid molecules in the sample by one or more nucleotides, one or more copy numbers, one or more transcript sequences, and/or one or more methylated residues.

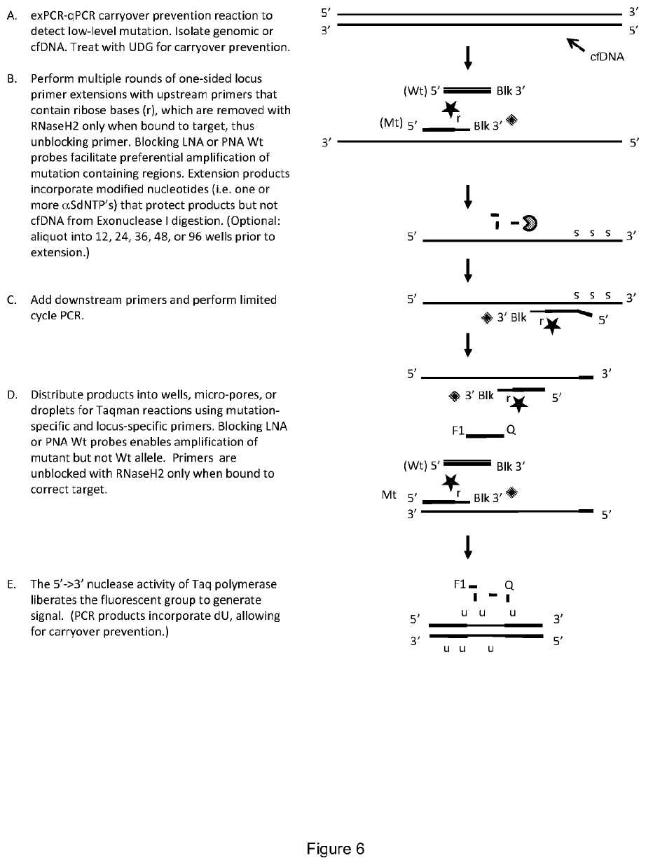

FIGS. 6, 7, and 8 illustrate various embodiments of this aspect of the present application.

FIG. 6 illustrates another exemplary exPCR-qPCR carryover prevention reaction to detect low-level mutations. Genomic or cfDNA is isolated (FIG. 6, step A), and the isolated DNA sample is treated with UDG to digest dU containing nucleic acid molecules that may be present in the sample (FIG. 6, step A). The sample is then subject to a linear amplification reaction, e.g., one or more polymerase extension reactions to generate complementary copies of mutation containing regions of interest. The regions of interest are selectively extended using locus-specific upstream primers, a blocking LNA or PNA probe comprising wild-type sequence, and a deoxynucleotide mix that includes one of more modified nucleotides. In this embodiment, another layer of selectivity can be incorporated into the method by including a 3′ cleavable blocking group (Blk 3′, e.g. C3 spacer), and an RNA base (r), in the upstream primer. Upon target-specific hybridization, RNase H (star symbol) removes the RNA base to liberate a 3′OH group which is a few bases upstream of the mutation, and suitable for polymerase extension (FIG. 6, step B). A blocking LNA or PNA probe comprising wild-type sequence that partially overlaps with the upstream PCR primer will preferentially compete in binding to wild-type sequence over the upstream primer, but not as much to mutant DNA, and thus suppresses extension of wild-type DNA during each round of primer extension. The initial extension products incorporate one or more modified nucleotides, such as α-thio-dNTPs, that protect the initial extension products (but not the original cfDNA or genomic DNA) from exonuclease I digestion (FIG. 6, step B). Optionally aliquot sample into 12, 24, 36, 48, or 96 wells prior to the initial extension step.

Subsequently, the locus-specific downstream primers are added, followed by limited cycle PCR (8 to 12 cycles, FIG. 6, step C). In the preferred embodiment, the locus-specific downstream primers are approximately 20 to 40 bases downstream from the locus-specific upstream primers. Optionally, the downstream primers contain identical 8-11 base tails to prevent primer dimers.

Following the limited cycle PCR, the PCR products are aliquoted into separate wells, micro-pores or droplets containing Taqman™ probes, mutation-specific, and locus-specific primers, to amplify the mutation-containing sequence, if present in the sample (FIG. 6, step D). Once again, the presence of blocking LNA or PNA probe comprising wild-type sequence suppresses extension of wild-type target sequence if present after the enrichment of mutant sequence during the initial extension-amplification steps (FIG. 6, steps B & C). As illustrated in step D of this FIG. 6, another layer of specificity can be incorporated into the method by including a 3′ cleavable blocking group (Blk 3′, e.g. C3 spacer), and an RNA base (r), in the mutation-specific and locus-specific primers. Upon target-specific hybridization, RNase H (star symbol) removes the RNA base to generate a polymerase extension competent 3′OH group (FIG. 6, step D). In the initial primer extension (step B), the liberated 3′OH is a few bases upstream from the mutation position, and thus would extend both wild-type and mutant sequences if cleaved (although the blocking LNA or PNA should limit cleavage of primer hybridized to wild-type sequence). In contrast, in the nested PCR (step D), the mutation-specific base of the primer is at the 3′OH, such that extension on wild-type sequence would be less likely, since the base is mismatched. The specificity for polymerase extension of mutant over wild-type sequence may be further improved by: (i) using mutation-specific PCR Primers containing a mismatch in the 2^{nd} or 3^{rd} position from the 3′OH base, (ii) using LNA or PNA probes to wild-type sequence that would reduce hybridization of mutation-specific PCR primers to wild-type sequences, (iii) using PCR primers to wild-type sequence that are blocked and do not undergo additional amplification, and (iv) avoiding G:T or T:G mismatches between primer and wild-type sequence at the 3′OH base. The TaqMan™ probe spans the mutation region and contains a fluorescent reporter group on one end (F1) and a quencher molecule (Q) on the other end that are in close enough proximity to each other in the intact probe that the quencher molecule quenches fluorescence of the reporter group. During amplification, the TaqMan™ probe and upstream primer hybridize to their complementary regions of the initial PCR product. The 5′→3′ nuclease activity of the polymerase extends the hybridized primer and liberates the fluorescent group of the TaqMan™ probe to generate a detectable signal (FIG. 6, step E). In a preferred embodiment, the Taqman™ probe contains a second quencher group (ZEN) about 9 bases in from the fluorescent reporter group, and the probe is designed such that the ZEN group is at or adjacent to the mutant base. Use of dUTP during the amplification reaction generates products containing dU, which can subsequently be destroyed using UDG for carryover prevention

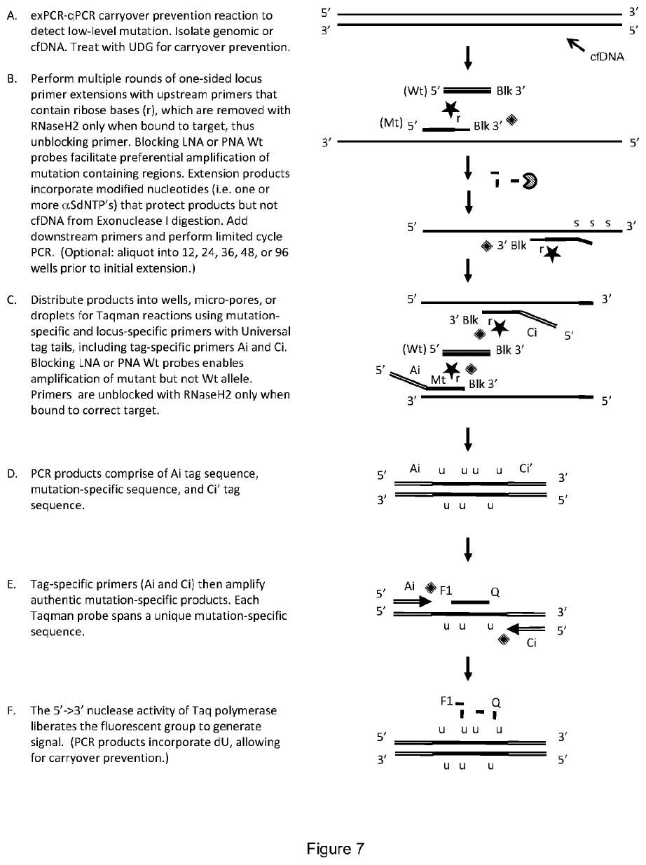

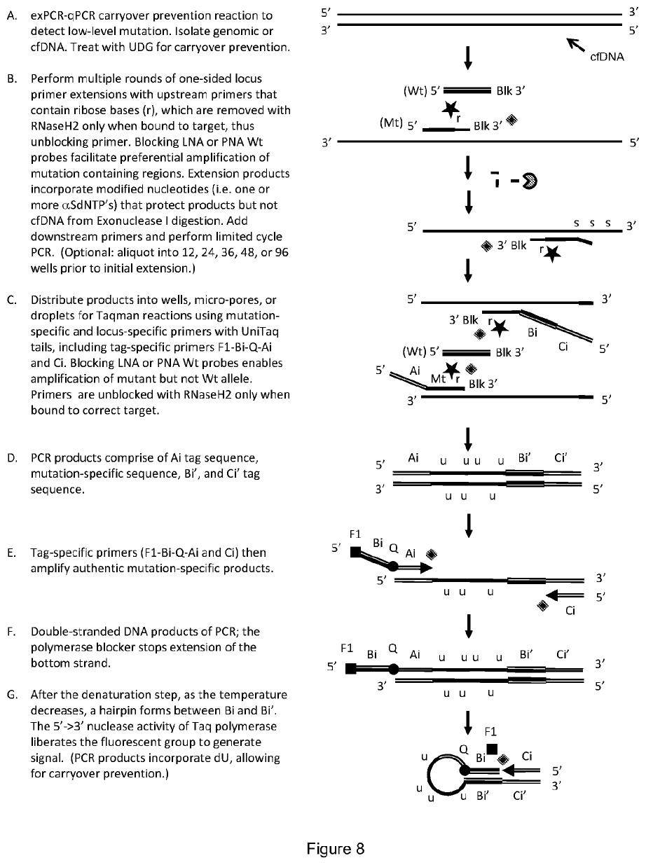

FIGS. 7 and 8 illustrate additional exemplary exPCR-qPCR carryover prevention reaction to detect low-level mutations. Genomic or cfDNA is isolated (FIGS. 7 and 8, step A), and the isolated DNA sample is treated with UDG to digest dU containing nucleic acid molecules that may be present in the sample (FIG. 7, step A). The regions of interest are selectively extended using locus-specific upstream primers, a blocking LNA or PNA probe comprising wild-type sequence, and a deoxynucleotide mix that includes one of more modified nucleotides. In this embodiment, another layer of selectivity can be incorporated into the method by including a 3′ cleavable blocking group (Blk 3′, e.g. C3 spacer), and an RNA base (r), in the upstream primer. Upon target-specific hybridization, RNase H (star symbol) removes the RNA base to liberate a 3′OH group which is a few bases upstream of the mutation, and suitable for polymerase extension (FIGS. 7 and 8, step B). A blocking LNA or PNA probe comprising wild-type sequence that partially overlaps with the upstream PCR primer will preferentially compete in binding to wild-type sequence over the upstream primer, but not as much to mutant DNA, and thus suppresses extension of wild-type DNA during each round of primer extension. The initial extension products incorporate one or more modified nucleotides, such as α-thio-dNTPs, that protect the initial extension products (but not the original cfDNA or genomic DNA) from exonuclease I digestion (FIGS. 7 and 8, step B). Optionally aliquot sample into 12, 24, 36, 48, or 96 wells prior to the initial extension step. Subsequently, the locus-specific downstream primers are added, followed by limited cycle PCR (8 to 12 cycles, FIGS. 7 and 8, step B).

For the protocol illustrated in FIG. 7, following the limited cycle PCR, the PCR products are aliquoted into separate wells, micro-pores or droplets containing Taqman™ probes, mutation-specific primers comprising 5′ primer-specific portions (Ai), locus-specific primers comprising 5′ primer-specific portions (Ci) and matching primers Ai and Ci. These primers combine to amplify the mutation-containing sequence, if present in the sample (FIG. 7, step C). Once again, the presence of blocking LNA or PNA probe comprising wild-type sequence suppresses extension of wild-type target sequence if present after the enrichment of mutant sequence during the initial extension-amplification steps (FIG. 7, step B). As illustrated in step C of this Figure, another layer of specificity can be incorporated into the method by including a 3′ cleavable blocking group (Blk 3′, e.g. C3 spacer), and an RNA base (r), in the mutation-specific and locus-specific primers. Upon target-specific hybridization, RNase H (star symbol) removes the RNA base to generate a polymerase extension competent 3′OH group (FIG. 7, step C). In the initial primer extension (step B) the liberated 3′OH is a few bases upstream from the mutation position, and thus would extend both wild-type and mutant sequences if cleaved (although the blocking LNA or PNA should limit cleavage of primer hybridized to wild-type sequence). In contrast, in the combined Taqman™—universal tag PCR amplification (steps C-F), the mutation-specific base of the upstream primer is at the 3′OH, such that extension on wild-type sequence would be less likely, since the base is mismatched. Following the mutation-specific and locus-specific extensions to generate products comprising the Ai tag sequence, target-specific sequence and Ci′ tag sequence (FIG. 7, step D), the products can be detected by the pairs of matched primers Ai and Ci, and TaqMan™ probes that span the ligation junction as described supra for FIG. 4 steps F-G (see FIG. 7, steps E & F), or using other suitable means known in the art. The specificity for polymerase extension of mutant over wild-type sequence may be further improved by: (i) using mutation-specific PCR Primers containing a mismatch in the 2^{nd} or 3^{rd} position from the 3′OH base, (ii) using LNA or PNA probes to wild-type sequence that would reduce hybridization of mutation-specific PCR primers to wild-type sequences, (iii) using PCR primers to wild-type sequence that are blocked and do not undergo additional amplification, and (iv) avoiding G:T or T:G mismatches between primer and wild-type sequence at the 3′OH base. Further, the longer target-specific primers are at a significantly lower concentration than the Taqman™ probe and tag-specific primers (Ai, Ci), such that the longer mutation-specific primers are depleted, allowing the Taqman™ probe and tag-specific primers to hybridize and enable target-dependent detection.

For the protocol illustrated in FIG. 8, following the limited cycle PCR, the PCR products are aliquoted into separate wells, micro-pores, or droplets containing Taqman™ probes, mutation-specific primers comprising 5′ primer-specific portions (Ai), locus-specific primers comprising 5′ primer-specific portions (Bi-Ci) and matching UniTaq primers F1-Bi-Q-Ai and Ci. These primers combine to amplify the mutation-containing sequence, if present in the sample (FIG. 8, step C). Once again, the presence of blocking LNA or PNA probe comprising wild-type sequence suppresses extension of wild-type target sequence if present after the enrichment of mutant sequence during the initial extension-amplification steps (FIG. 8, step B). As illustrated in step C of this Figure, another layer of specificity can be incorporated into the method by including a 3′ cleavable blocking group (Blk 3′, e.g. C3 spacer), and an RNA base (r), in the mutation-specific and locus-specific primers. Upon target-specific hybridization, RNase H (star symbol) removes the RNA base to generate a polymerase extension competent 3′OH group (FIG. 8, step C). In the initial primer extension (step B) the liberated 3′OH is a few bases upstream from the mutation position, and thus would extend both wild-type and mutant sequences if cleaved (although the blocking LNA or PNA should limit cleavage of primer hybridized to wild-type sequence). In contrast, in the combined Taqman™—UniTaq PCR amplification (steps C-G), the mutation-specific base of the upstream primer is at the 3′OH, such that extension on wild-type sequence would be less likely, since the base is mismatched. Following the mutation-specific and locus-specific extensions to generate products comprising the Ai tag sequence, target-specific sequence and Bi′-Ci′ tag sequence (FIG. 8, step D), the products can be detected by the pairs of matched UniTaq primers (i.e. F1-Bi-Q-Ai and Ci), as described supra for FIG. 5 steps F-H (see FIG. 8, steps E-G), or using other suitable means known in the art. The specificity for polymerase extension of mutant over wild-type sequence may be further improved by: (i) using mutation-specific PCR Primers containing a mismatch in the 2^{nd} or 3^{rd} position from the 3′OH base, (ii) using LNA or PNA probes to wild-type sequence that would reduce hybridization of mutation-specific PCR primers to wild-type sequences, (iii) using PCR primers to wild-type sequence that are blocked and do not undergo additional amplification, and (iv) avoiding G:T or T:G mismatches between primer and wild-type sequence at the 3′OH base. Further, the longer target-specific primers are at a significantly lower concentration than the UniTaq primers (F1-Bi-Q-Ai, Ci), such that the longer mutation-specific primers are depleted, allowing the UniTaq primers to hybridize and enable target-dependent detection.

Another aspect of the present application is directed to a method for identifying, in a sample, one or more parent nucleic acid molecules containing a target nucleotide sequence differing from nucleotide sequences in other parent nucleic acid molecules in the sample by one or more methylated residues. The method involves providing a sample containing one or more parent nucleic acid molecules potentially containing the target nucleotide sequence differing from the nucleotide sequences in other parent nucleic acid molecules by one or more methylated residues and subjecting the nucleic acid molecules in the sample to a bisulfite treatment under conditions suitable to convert unmethylated cytosine residues to uracil residues. One or more enzymes capable of digesting deoxyuracil (dU)-containing nucleic acid molecules are then provided. The method further involves providing one or more primary oligonucleotide primer sets. Each primary oligonucleotide primer set comprises (a) a first primary oligonucleotide primer that comprises a nucleotide sequence that is complementary to a sequence in the bisulfite-treated parent nucleic acid molecules adjacent to the bisulfite-treated target nucleotide sequence containing the one or more methylated residue and (b) a second primary oligonucleotide primer that comprises a nucleotide sequence that is complementary to a portion of an extension product formed from the first primary oligonucleotide primer. The bisulfite-treated sample, the one or more first primary oligonucleotide primers of the one or more primary oligonucleotide primer sets, a deoxynucleotide mix, and a DNA polymerase are blended to form one or more polymerase extension reaction mixtures, and the one or more polymerase extension reaction mixtures are subjected to conditions suitable for one or more polymerase extension reaction cycles comprising a denaturation treatment, a hybridization treatment, and an extension treatment, thereby forming primary extension products comprising the complement of the bisulfite-treated target nucleotide sequence. The one or more polymerase extension reaction mixtures comprising the primary extension products, the one or more secondary primary oligonucleotide primers of the one or more primary oligonucleotide primer sets, the one or more enzymes capable of digesting deoxyuracil (dU)-containing nucleic acid molecules, a deoxynucleotide mix including dUTP, and a DNA polymerase are blended to form one or more first polymerase chain reaction mixtures. The one or more first polymerase chain reaction mixtures are subjected to conditions suitable for digesting deoxyuracil (dU)-containing nucleic acid molecules present in the first polymerase chain reaction mixtures and for carrying out one or more polymerase chain reaction cycles comprising a denaturation treatment, a hybridization treatment, and an extension treatment, thereby forming first polymerase chain reaction products comprising the bisulfite-treated target nucleotide sequence or a complement thereof. The method further involves providing one or more oligonucleotide probe sets. Each probe set comprises (a) a first oligonucleotide probe having a 5′ primer-specific portion and a 3′ bisulfite-treated target nucleotide sequence-specific or complement sequence-specific portion, and (b) a second oligonucleotide probe having a 5′ bisulfite-treated target nucleotide sequence-specific or complement sequence-specific portion and a 3′ primer-specific portion, and wherein the first and second oligonucleotide probes of a probe set are configured to hybridize, in a base specific manner, on a complementary nucleotide sequence of a first polymerase chain reaction product. The first polymerase chain reaction products are blended with a ligase and the one or more oligonucleotide probe sets to form one or more ligation reaction mixtures. The one or more ligation reaction mixtures are subjected to one or more ligation reaction cycles whereby the first and second oligonucleotide probes of the one or more oligonucleotide probe sets are ligated together, when hybridized to complementary sequences, to form ligated product sequences in the ligation reaction mixture wherein each ligated product sequence comprises the 5′ primer-specific portion, the bisulfite-treated target nucleotide sequence-specific or complement sequence-specific portions, and the 3′ primer-specific portion. The method further comprises providing one or more secondary oligonucleotide primer sets. Each secondary oligonucleotide primer set comprises (a) a first secondary oligonucleotide primer comprising the same nucleotide sequence as the 5′ primer-specific portion of the ligated product sequence and (b) a second secondary oligonucleotide primer comprising a nucleotide sequence that is complementary to the 3′ primer-specific portion of the ligated product sequence. The ligated product sequences, the one or more secondary oligonucleotide primer sets, the one or more enzymes capable of digesting deoxyuracil (dU)-containing nucleic acid molecules, a deoxynucleotide mix including dUTP, and a DNA polymerase are blended to form one or more second polymerase chain reaction mixtures, and the one or more second polymerase chain reaction mixtures are subjected to conditions suitable for digesting deoxyuracil (dU)-containing nucleic acid molecules present in the second polymerase chain reaction mixtures and for carrying out one or more polymerase chain reaction cycles comprising a denaturation treatment, a hybridization treatment, and an extension treatment thereby forming a second polymerase chain reaction products. The method further involves detecting and distinguishing the second polymerase chain reaction products in the one or more second polymerase chain reaction mixtures to identify the presence of one or more nucleic acid molecules containing target nucleotide sequences differing from nucleotide sequences in other parent nucleic acid molecules in the sample by one or more methylated residues.

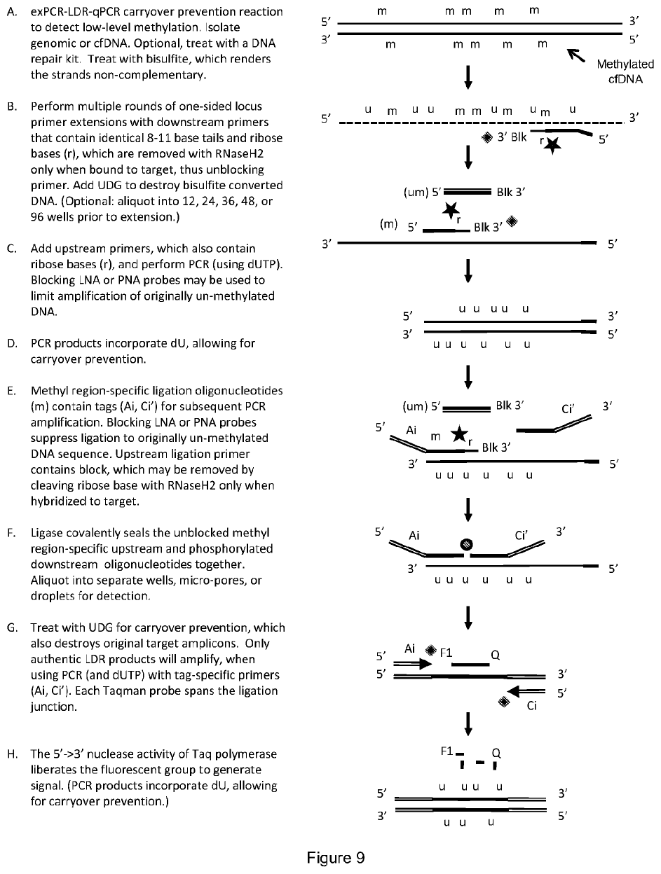

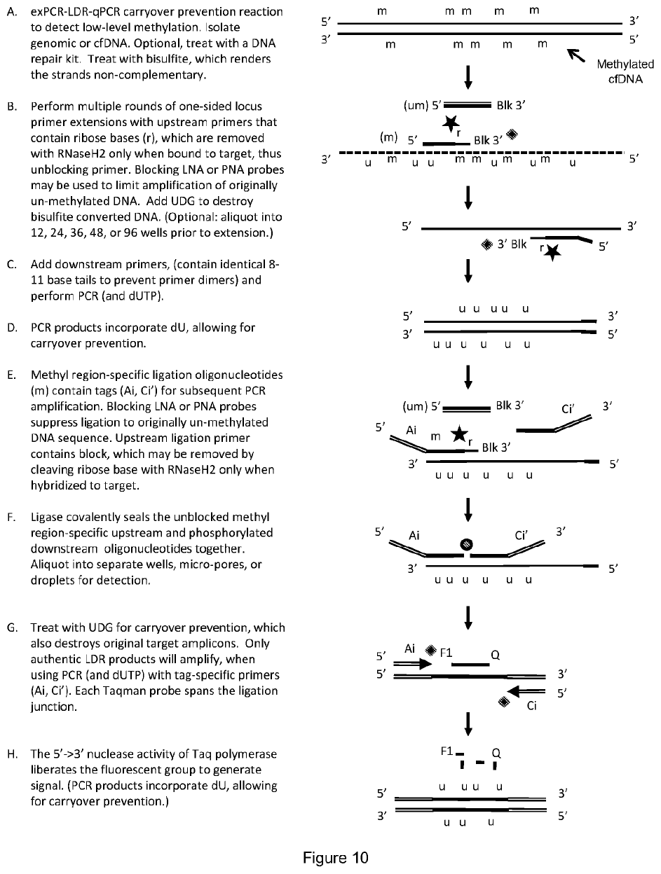

FIGS. 9 and 10 illustrate exPCR-LDR-qPCR carryover prevention reaction to detect low-level methylation in accordance with this aspect of the present application. The steps are similar to those steps described for FIG. 2, with two key differentiators. First, after isolating the genomic or cfDNA, it is optionally treated with a DNA repair kit prior to bisulfite conversion (FIGS. 9 and 10, Step A). Bisulfite converts unmethylated cytosines, but not 5-methyl cytosines (5meC) nor 5-hydroxymethyl cytosine (5hmC) into a uracil base, which base-pairs with A. Thus, after a single cycle of PCR amplification, unmethylated Cm but not 5meC nor 5hmC is converted to a “T” base, thus allowing for both modified forms of cytosine to be distinguished from unmodified cytosine. Second, the regions of interest are selectively extended using locus-specific downstream primers (with optional identical 8-11 base tails), and a deoxynucleotide mix that does NOT include dUTP. In this embodiment, another layer of selectivity can be incorporated into the method by including a 3′ cleavable blocking group (Blk 3′, e.g. C3 spacer), and an RNA base (r), in the downstream primer. Upon target-specific hybridization, RNase H (star symbol) removes the RNA base to liberate a 3′OH group which is suitable for polymerase extension (FIG. 9, step B). Add UDG, which destroys the bisulfite converted DNA (but not the primer extension products). Optionally aliquot sample into 12, 24, 36, 48, or 96 wells prior to the initial extension step. Subsequently, the locus-specific upstream primers are added, followed by limited (8 to 20 cycles) or full (20-40 cycles) PCR using a deoxynucleotide mix that includes dUTP (FIG. 9, step C). Upon target-specific hybridization, RNase H removes the RNA base to liberate a 3′OH group which is a few bases upstream of the bisulfite converted methylated target base, and suitable for polymerase extension (FIG. 9, step C). A blocking LNA or PNA probe comprising the bisulfite converted unmethylated sequence (or its complement) that partially overlaps with the upstream PCR primer will preferentially compete for binding to the bisulfite converted unmethylated sequence over the upstream primer, thus suppressing amplification of bisulfite converted unmethylated sequence DNA during each round of PCR. Optionally, the downstream primers contain identical 8-11 base tails to prevent primer dimers. Further, such tails provide the option for asymmetric PCR at the end of the PCR cycles, by raising the hybridization temperature above that for the forward primers, but at or below that for the reverse primers—which at 8-11 bases longer will have higher Tm values. This generates more bottom strand products, which are suitable substrates for the subsequent LDR step. The amplified products contain dU as shown in FIG. 9, step D, which allows for subsequent treatment with UDG or a similar enzyme for carryover prevention.

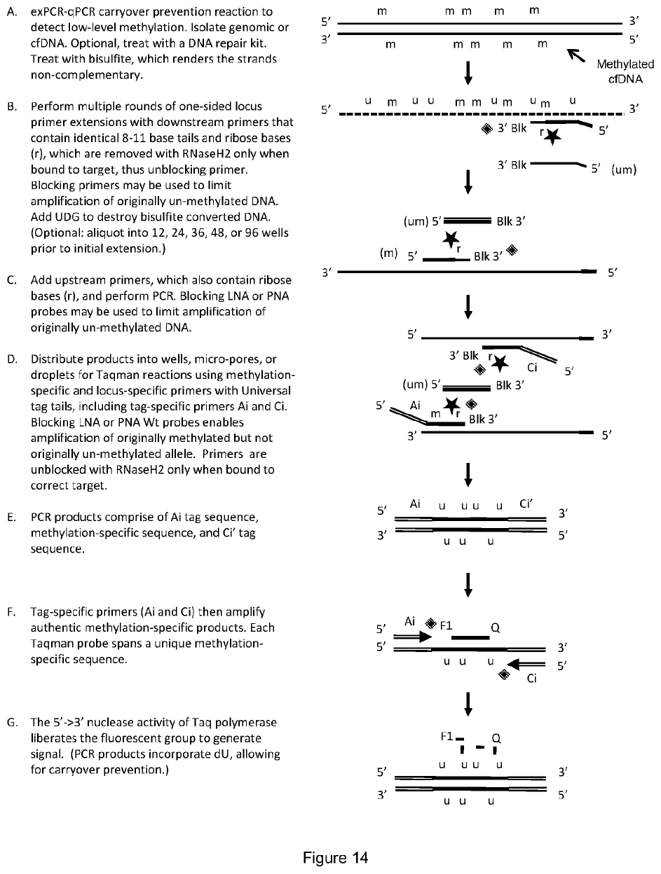

Alternatively, as shown in FIG. 10, the regions of interest are selectively extended using locus-specific upstream primers, a blocking LNA or PNA probe comprising bisulfite converted unmethylated sequence (or its complement), and a deoxynucleotide mix that does NOT include dUTP. In this embodiment, another layer of selectivity can be incorporated into the method by including a 3′ cleavable blocking group (Blk 3′, e.g. C3 spacer), and an RNA base (r), in the upstream primer. Upon target-specific hybridization, RNase H (star symbol) removes the RNA base to liberate a 3′OH group which is a few bases upstream of the bisulfite converted methylated target base, and suitable for polymerase extension (FIG. 10, step B). A blocking LNA or PNA probe comprising the bisulfite converted unmethylated sequence (or its complement) that partially overlaps with the upstream PCR primer will preferentially compete for binding to the bisulfite converted unmethylated sequence over the upstream primer, thus suppressing amplification of bisulfite converted unmethylated sequence DNA during each round of PCR. Add UDG, which destroys the bisulfite converted DNA (but not the primer extension products). Optionally aliquot sample into 12, 24, 36, 48, or 96 wells prior to the initial extension step. Subsequently, the locus-specific downstream primers are added, followed by limited (8 to 20 cycles) or full (20-40 cycles) PCR using a deoxynucleotide mix that includes dUTP (FIG. 14, step C). Optionally, the downstream primers contain identical 8-11 base tails to prevent primer dimers.

For FIGS. 9 and 10, methylation-specific upstream and locus-specific downstream probes containing tails (Ai, Ci′) enable formation of a ligation product in the presence of bisulfite converted methylated base-containing PCR products. Following ligation, the ligation products can be detected using pairs of matched primers Ai and Ci, and TaqMan™ probes that span the ligation junction as described supra for FIG. 2 (see FIG. 9, steps E-H), or using other suitable means known in the art.

Alternatively, methylation-specific upstream and locus-specific downstream probes containing tails (Ai, Bi′-Ci′) enable formation of a ligation product in the presence of bisulfite converted methylated base-containing PCR products. Following ligation, the ligation products are amplified using UniTaq-specific primers (i.e., F1-Bi-Q-Ai, Ci) and detected as described supra for FIG. 3, or using other suitable means known in the art.

Another aspect of the present application is directed to a method for identifying, in a sample, one or more parent nucleic acid molecules containing a target nucleotide sequence differing from nucleotide sequences in other parent nucleic acid molecules in the sample by one or more methylated residues. The method involves providing a sample containing one or more parent nucleic acid molecules potentially containing the target nucleotide sequence differing from the nucleotide sequences in other parent nucleic acid molecules by one or more methylated residues. The nucleic acid molecules in the sample are subjected to a bisulfite treatment under conditions suitable to convert unmethylated cytosine residues to uracil residues. One or more enzymes capable of digesting deoxyuracil (dU)-containing nucleic acid molecules are provided, and one or more first primary oligonucleotide primer(s) are provided. Each first primary oligonucleotide primer comprises a nucleotide sequence that is complementary to a sequence in the bisulfite-treated parent nucleic acid molecules adjacent to the bisulfite-treated target nucleotide sequence containing the one or more methylated residue. The bisulfite-treated sample, the one or more first primary oligonucleotide primers, a deoxynucleotide mix, and a DNA polymerase are blended to form one or more polymerase extension reaction mixtures, and the one or more polymerase extension reaction mixtures are subjected to conditions suitable for one or more polymerase extension reaction cycles comprising a denaturation treatment, a hybridization treatment, and an extension treatment, to form primary extension products comprising the complement of the bisulfite-treated target nucleotide sequence. The method further comprises providing one or more secondary oligonucleotide primer sets. Each secondary oligonucleotide primer set comprises (a) a first secondary oligonucleotide primer having a 5′ primer-specific portion and a 3′ portion that is complementary to a portion of the polymerase extension reaction product formed from the first primary oligonucleotide primer and (b) a second secondary oligonucleotide primer having a 5′ primer-specific portion and a 3′ portion that comprises a nucleotide sequence that is complementary to a portion of an extension product formed from the first secondary oligonucleotide primer. The one or more polymerase extension reaction mixtures comprising the primary extension products, the one or more secondary oligonucleotide primer sets, the one or more enzymes capable of digesting deoxyuracil (dU)-containing nucleic acid molecules, a deoxynucleotide mix, and a DNA polymerase are blended to form one or more first polymerase chain reaction mixtures, and the one or more first polymerase chain reaction mixtures are subjected to conditions suitable for digesting nucleic acid molecules present in the first polymerase chain reaction mixtures, but not primary extension products comprising modified nucleotides and for carrying out two or more polymerase chain reaction cycles comprising a denaturation treatment, a hybridization treatment, and an extension treatment, thereby forming first polymerase chain reactions products comprising a 5′ primer-specific portion of the first secondary oligonucleotide primer, the bisulfite-treated target nucleotide sequence-specific or complement sequence-specific portion, and a complement of the 5′ primer-specific portion of the second secondary oligonucleotide primer. The method further involves providing one or more tertiary oligonucleotide primer sets. Each tertiary oligonucleotide primer set comprises (a) a first tertiary oligonucleotide primer comprising the same nucleotide sequence as the 5′ primer-specific portion of the first polymerase chain reaction products and (b) a second tertiary oligonucleotide primer comprising a nucleotide sequence that is complementary to the 3′ primer-specific portion of the first polymerase chain reactions product sequence. The first polymerase chain reaction products, the one or more tertiary oligonucleotide primer sets, the one or more enzymes capable of digesting deoxyuracil (dU) containing nucleic acid molecules, a deoxynucleotide mix including dUTP, and a DNA polymerase are blended to form one or more second polymerase chain reaction mixtures. The one or more second polymerase chain reaction mixtures are subjected to conditions suitable for digesting deoxyuracil (dU)-containing nucleic acid molecules present in the second polymerase chain reaction mixtures and for carrying out one or more polymerase chain reaction cycles comprising a denaturation treatment, a hybridization treatment, and an extension treatment thereby forming secondary polymerase chain reaction products. The method further involves detecting and distinguishing the secondary polymerase chain reactions products in the one or more second polymerase chain reaction mixtures to identify the presence of one or more parent nucleic acid molecules containing target nucleotide sequences differing from nucleotide sequences in other parent nucleic acid molecules in the sample by one or more methylated residues.

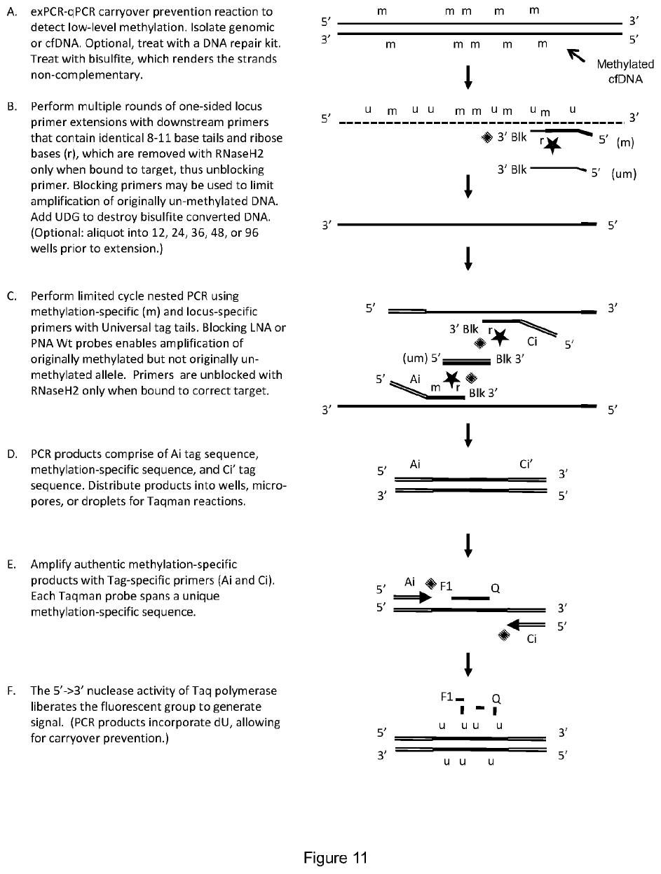

FIGS. 11, 12, 18, and 19 illustrate various embodiments of this aspect of the present application.

FIG. 11 illustrates an exemplary exPCR-qPCR carryover prevention reaction to detect low-level methylations. Genomic or cfDNA is isolated and is optionally treated with a DNA repair kit prior to bisulfite conversion (FIG. 11, Step A). The regions of interest are selectively extended using locus-specific downstream primers (with optional identical 8-11 base tails), and a deoxynucleotide mix that does NOT include dUTP. In this embodiment, another layer of selectivity can be incorporated into the method by including a 3′ cleavable blocking group (Blk 3′, e.g. C3 spacer), and an RNA base (r), in the downstream primer. Upon target-specific hybridization, RNase H (star symbol) removes the RNA base to liberate a 3′OH group which is suitable for polymerase extension (FIG. 11, step B). If the locus-specific downstream primer covers one or more methylation sites, another layer of specificity may be added by using blocking primers whose sequence corresponds to the bisulfite-converted unmethylated sequence. After the extension cycles, add UDG, which destroys the bisulfite converted DNA (but not the primer extension products). Optionally aliquot sample into 12, 24, 36, 48, or 96 wells prior to the initial extension step.

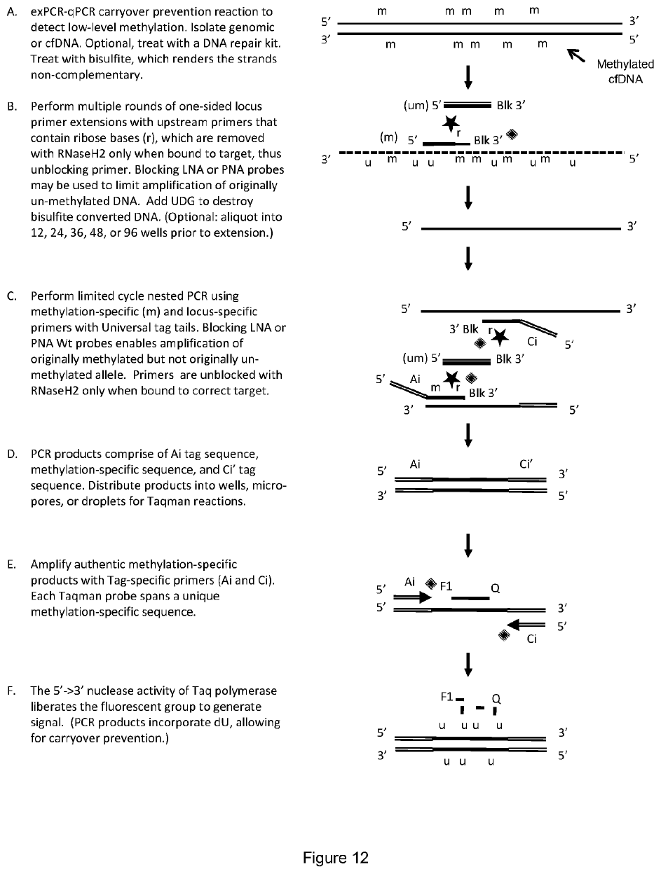

Alternatively, as shown in FIG. 12, regions of interest are selectively extended using locus-specific upstream primers, a blocking LNA or PNA probe comprising bisulfite converted unmethylated sequence (or its complement), and a deoxynucleotide mix that does not include dUTP. In this embodiment, another layer of selectivity can be incorporated into the method by including a 3′ cleavable blocking group (Blk 3′, e.g. C3 spacer), and an RNA base (r), in the upstream primer. Upon target-specific hybridization, RNase H (star symbol) removes the RNA base to liberate a 3′OH group which is a few bases upstream of the bisulfite converted methylated target base, and suitable for polymerase extension (FIG. 12, step B). A blocking LNA or PNA probe comprising the bisulfite converted unmethylated sequence (or its complement) that partially overlaps with the upstream PCR primer will preferentially compete for binding to the bisulfite converted unmethylated sequence over the upstream primer, thus suppressing amplification of bisulfite converted unmethylated sequence DNA during each round of PCR. Add UDG, which destroys the bisulfite converted DNA (but not the primer extension products). Optionally aliquot sample into 12, 24, 36, 48, or 96 wells prior to the initial extension step.

As shown in FIGS. 11 and 12 step C, bisulfite converted methylation base-specific primers (comprising 5′ primer-specific portions Ai) and bisulfite converted locus-specific primers (comprising 5′ primer-specific portions Ci) are added to then perform limited cycle nested PCR to amplify the bisulfite converted methylation-containing sequence, if present in the sample. Blocking LNA or PNA probes comprising the bisulfite converted unmethylated sequence (or its complement) enables amplification of originally methylated but not originally un-methylated allele. Primers are unblocked with RNaseH2 only when bound to correct target. Following PCR, the products can be detected using pairs of matched primers Ai and Ci, and TaqMan™ probes that span the bisulfite-converted methylation target regions as described supra for FIG. 4 (see FIGS. 11 and 12, steps D-F), or using other suitable means known in the art.

Alternatively, bisulfite converted methylation base-specific primers (comprising 5′ primer-specific portions Ai) and bisulfite converted locus-specific primers (comprising 5′ primer-specific portions Bi-Ci) are added to then perform limited cycle nested PCR to amplify the bisulfite converted methylation-containing sequence, if present in the sample. Blocking LNA or PNA probes comprising the bisulfite converted unmethylated sequence (or its complement) enables amplification of originally methylated but not originally un-methylated alleles. Primers are unblocked with RNaseH2 only when bound to correct target. Following PCR, the products are amplified using UniTaq-specific primers (i.e., F1-Bi-Q-Ai, Ci) and detected as described supra for FIG. 5, or using other suitable means known in the art.

Another aspect of the present application is directed to a method for identifying, in a sample, one or more parent nucleic acid molecules containing a target nucleotide sequence differing from nucleotide sequences in other parent nucleic acid molecules in the sample by one or more methylated residues. The method involves providing a sample containing one or more parent nucleic acid molecules potentially containing the target nucleotide sequence differing from the nucleotide sequences in other parent nucleic acid molecules by one or more methylated residues. The nucleic acid molecules in the sample are subjected to a bisulfite treatment under conditions suitable to convert unmethylated cytosine residues to uracil residues. One or more enzymes capable of digesting deoxyuracil (dU)-containing nucleic acid molecules present in the sample are provided, and one or more primary oligonucleotide primer sets are provided. Each primary oligonucleotide primer set comprises (a) a first primary oligonucleotide primer that comprises a nucleotide sequence that is complementary to a sequence in the bisulfite-treated parent nucleic acid molecules adjacent to the bisulfite-treated target nucleotide sequence containing the one or more methylated residue and (b) a second primary oligonucleotide primer that comprises a nucleotide sequence that is complementary to a portion of an extension product formed from the first primary oligonucleotide primer. The bisulfite-treated sample, the one or more first primary oligonucleotide primers of the one or more primary oligonucleotide primer sets, a deoxynucleotide mix, and a DNA polymerase are blended to form one or more polymerase extension reaction mixtures. The one or more polymerase extension reaction mixtures to are subjected to conditions suitable for one or more polymerase extension reaction cycles comprising a denaturation treatment, a hybridization treatment, and an extension treatment, thereby forming primary extension products comprising the complement of the bisulfite treated target nucleotide sequence. The one or more polymerase extension reaction mixtures comprising the primary extension products, the one or more secondary primary oligonucleotide primers of the one or more primary oligonucleotide primer sets, the one or more enzymes capable of digesting deoxyuracil (dU)-containing nucleic acid molecules in the reaction mixture, a deoxynucleotide mix, and a DNA polymerase are blended to form one or more first polymerase chain reaction mixtures. The one or more first polymerase chain reaction mixtures are subjected to conditions suitable for digesting deoxyuracil (dU)-containing nucleic acid molecules present in the first polymerase chain reaction mixtures and for carrying out one or more polymerase chain reaction cycles comprising a denaturation treatment, a hybridization treatment, and an extension treatment, thereby forming first polymerase chain reaction products comprising the bisulfite-treated target nucleotide sequence or a complement thereof. The method further comprises providing one or more secondary oligonucleotide primer sets. Each secondary oligonucleotide primer set comprises (a) a first secondary oligonucleotide primer having a 3′ portion that is complementary to a portion of a first polymerase chain reaction product formed from the first primary oligonucleotide primer and (b) a second secondary oligonucleotide primer having a 3′ portion that comprises a nucleotide sequence that is complementary to a portion of a first polymerase chain reaction product formed from the first secondary oligonucleotide primer. The first polymerase chain reaction products, the one or more secondary oligonucleotide primer sets, the one or more enzymes capable of digesting deoxyuracil (dU)-containing nucleic acid molecules, a deoxynucleotide mix including dUTP, and a DNA polymerase are blended to form one or more second polymerase chain reaction mixtures. The one or more second polymerase chain reaction mixtures are subjected to conditions suitable for digesting deoxyuracil (dU)-containing nucleic acid molecules present in the second polymerase chain reaction mixtures and for carrying out two or more polymerase chain reaction cycles comprising a denaturation treatment, a hybridization treatment, and an extension treatment thereby forming second polymerase chain reaction products. The method further involves detecting and distinguishing the second polymerase chain reactions products in the one or more second polymerase chain reaction mixtures to identify the presence of one or more parent nucleic acid molecules containing target nucleotide sequences differing from nucleotide sequences in other parent nucleic acid molecules in the sample by one or more methylated residues.

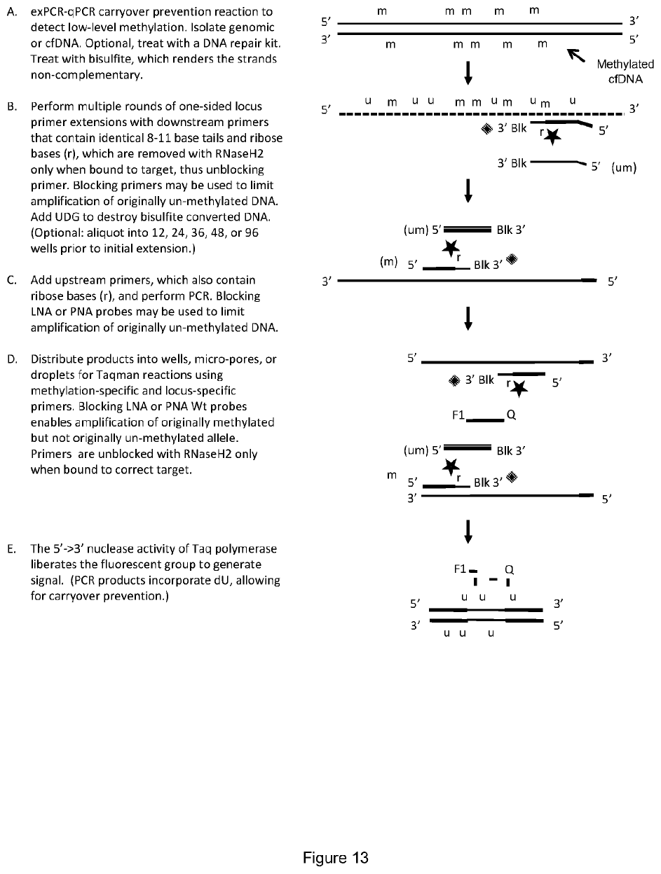

FIGS. 13-15, 20, and 21 illustrate various embodiments of this aspect of the present application.

FIG. 13 illustrates another exemplary exPCR-qPCR carryover prevention reaction to detect low-level methylation. Genomic or cfDNA is isolated, and optionally treated with a DNA repair kit prior to bisulfite conversion (FIG. 13, Step A). The regions of interest are selectively extended using locus-specific downstream primers (with optional identical 8-11 base tails), and a deoxynucleotide mix that does NOT include dUTP. In this embodiment, another layer of selectivity can be incorporated into the method by including a 3′ cleavable blocking group (Blk 3′, e.g. C3 spacer), and an RNA base (r), in the downstream primer. Upon target-specific hybridization, RNase H (star symbol) removes the RNA base to liberate a 3′OH group which is suitable for polymerase extension (FIG. 13, step B). If the locus-specific downstream primer covers one or more methylation sites, another layer of specificity may be added by using blocking primers whose sequence corresponds to the bisulfite-converted unmethylated sequence. After the extension cycles, add UDG, which destroys the bisulfite converted DNA (but not the primer extension products). Optionally aliquot sample into 12, 24, 36, 48, or 96 wells prior to the initial extension step. Subsequently, the regions of interest are selectively amplified in a limited cycle PCR (8-20 cycles) using locus-specific upstream primers, a blocking LNA or PNA probe comprising bisulfite converted unmethylated sequence (or its complement), and a deoxynucleotide mix that does not include dUTP. In this embodiment, another layer of selectivity can be incorporated into the method by including a 3′ cleavable blocking group (Blk 3′, e.g. C3 spacer), and an RNA base (r), in the upstream primer. Upon target-specific hybridization, RNase H (star symbol) removes the RNA base to liberate a 3′OH group which is a few bases upstream of the bisulfite converted methylated target base, and suitable for polymerase extension (FIG. 13, step C). A blocking LNA or PNA probe comprising the bisulfite converted unmethylated sequence (or its complement) that partially overlaps with the upstream PCR primer will preferentially compete for binding to the bisulfite converted unmethylated sequence over the upstream primer, thus suppressing amplification of bisulfite converted unmethylated sequence DNA during each round of PCR.

Following the limited cycle PCR, the PCR products are aliquoted into separate wells, micro-pores or droplets containing Taqman™ probes, bisulfite-converted, methylation base-specific, and bisulfite converted locus-specific primers, to amplify the bisulfite converted methylation-containing sequence, if present in the sample (FIG. 13, step D) The bisulfite converted methylation-containing products are amplified and detected as described supra for FIG. 6 (see FIG. 13, steps D-E), or using other suitable means known in the art.

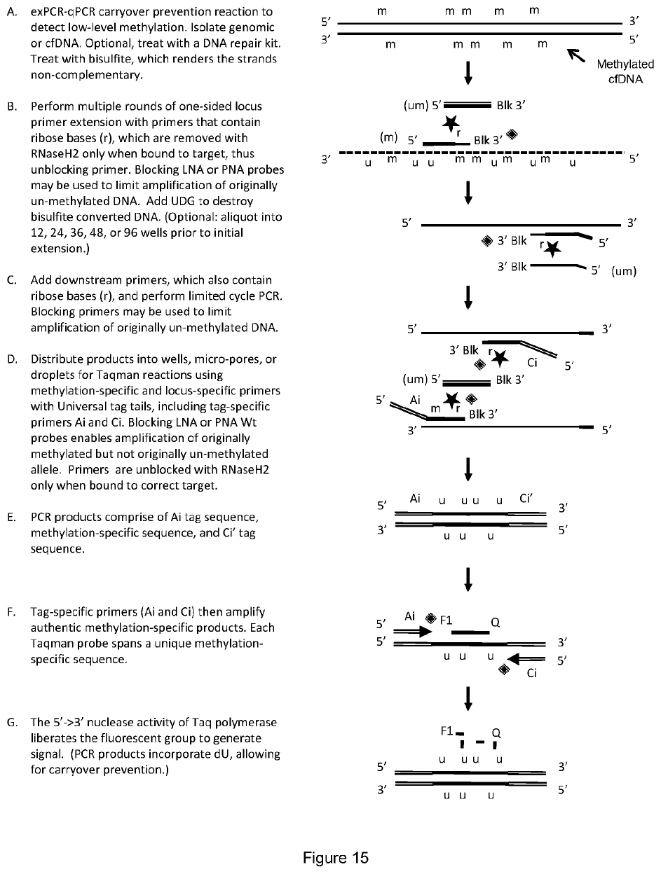

FIGS. 14 and 15 illustrate additional exemplary exPCR-qPCR carryover prevention reaction to detect low-level methylation. Genomic or cfDNA is isolated, and optionally treated with a DNA repair kit prior to bisulfite conversion (FIGS. 14 and 15, Step A). The regions of interest are selectively extended using locus-specific downstream primers (with optional identical 8-11 base tails), and a deoxynucleotide mix that does NOT include dUTP. In this embodiment, another layer of selectivity can be incorporated into the method by including a 3′ cleavable blocking group (Blk 3′, e.g. C3 spacer), and an RNA base (r), in the downstream primer. Upon target-specific hybridization, RNase H (star symbol) removes the RNA base to liberate a 3′ OH group which is suitable for polymerase extension (FIG. 14, step B). If the locus-specific downstream primer covers one or more methylation sites, another layer of specificity may be added by using blocking primers whose sequence corresponds to the bisulfite-converted unmethylated sequence. After the extension cycles, add UDG, which destroys the bisulfite converted DNA (but not the primer extension products). Optionally aliquot sample into 12, 24, 36, 48, or 96 wells prior to the initial extension step. Subsequently, the regions of interest are selectively amplified in a limited cycle PCR (8-20 cycles) using locus-specific upstream primers, a blocking LNA or PNA probe comprising bisulfite converted unmethylated sequence (or its complement), and a deoxynucleotide mix that does not include dUTP. In this embodiment, another layer of selectivity can be incorporated into the method by including a 3′ cleavable blocking group (Blk 3′, e.g. C3 spacer), and an RNA base (r), in the upstream primer. Upon target-specific hybridization, RNase H (star symbol) removes the RNA base to liberate a 3′OH group which is a few bases upstream of the bisulfite converted methylated target base, and suitable for polymerase extension (FIG. 14, step C). A blocking LNA or PNA probe comprising the bisulfite converted unmethylated sequence (or its complement) that partially overlaps with the upstream PCR primer will preferentially compete for binding to the bisulfite converted unmethylated sequence over the upstream primer, thus suppressing amplification of bisulfite converted unmethylated sequence DNA during each round of PCR.

Alternatively, as shown in FIG. 15, the regions of interest are selectively extended using locus-specific upstream primers, a blocking LNA or PNA probe comprising bisulfite converted unmethylated sequence (or its complement), and a deoxynucleotide mix that does not include dUTP. In this embodiment, another layer of selectivity can be incorporated into the method by including a 3′ cleavable blocking group (Blk 3′, e.g. C3 spacer), and an RNA base (r), in the upstream primer. Upon target-specific hybridization, RNase H (star symbol) removes the RNA base to liberate a 3′OH group which is a few bases upstream of the bisulfite converted methylated target base, and suitable for polymerase extension (FIG. 15, step B). A blocking LNA or PNA probe comprising the bisulfite converted unmethylated sequence (or its complement) that partially overlaps with the upstream PCR primer will preferentially compete for binding to the bisulfite converted unmethylated sequence over the upstream primer, thus suppressing amplification of bisulfite converted unmethylated sequence DNA during each round of PCR. Add UDG, which destroys the bisulfite converted DNA (but not the primer extension products). Subsequently, the locus-specific downstream primers are added, followed by limited cycle PCR (8 to 12 cycles, FIG. 15, step C). If the locus-specific downstream primer covers one or more methylation sites, another layer of specificity may be added by using blocking primers whose sequence corresponds to the bisulfite-converted unmethylated sequence. Optionally aliquot sample into 12, 24, 36, 48, or 96 wells prior to the initial extension step.

For the protocol illustrated in FIGS. 14 and 15, following the limited cycle PCR, the PCR products are aliquoted into separate wells, micro-pores or droplets containing Taqman™ probes, bisulfite converted methylation base-specific primers comprising 5′ primer-specific portions (Ai), bisulfite converted locus-specific primers comprising 5′ primer-specific portions (Ci) and matching primers Ai and Ci. These primers combine to amplify the bisulfite converted methylation-containing sequence, if present in the sample (FIGS. 14 and 15, step D) Blocking LNA or PNA probes comprising the bisulfite converted unmethylated sequence (or its complement) enables amplification of originally methylated but not originally un-methylated allele. Primers are unblocked with RNaseH2 only when bound to correct target. Following PCR, the products can be detected using pairs of matched primers Ai and Ci, and TaqMan™ probes that span the bisulfite-converted methylation target regions as described supra for FIG. 4 (see FIG. 14, steps E-G), or using other suitable means known in the art.

Alternatively, following the limited cycle PCR, the PCR products are aliquoted into separate wells, micro-pores or droplets containing Taqman™ probes, bisulfite converted methylation base-specific primers comprising 5′ primer-specific portions (Ai), bisulfite converted locus-specific primers comprising 5′ primer-specific portions (Bi-Ci) and matching UniTaq primers F1-Bi-Q-Ai and Ci. Blocking LNA or PNA probes comprising the bisulfite converted unmethylated sequence (or its complement) enables amplification of originally methylated but not originally un-methylated alleles. Primers are unblocked with RNaseH2 only when bound to correct target. Following PCR, the products are amplified using UniTaq-specific primers (i.e., F1-Bi-Q-Ai, Ci) and detected as described supra for FIG. 5, or using other suitable means known in the art.

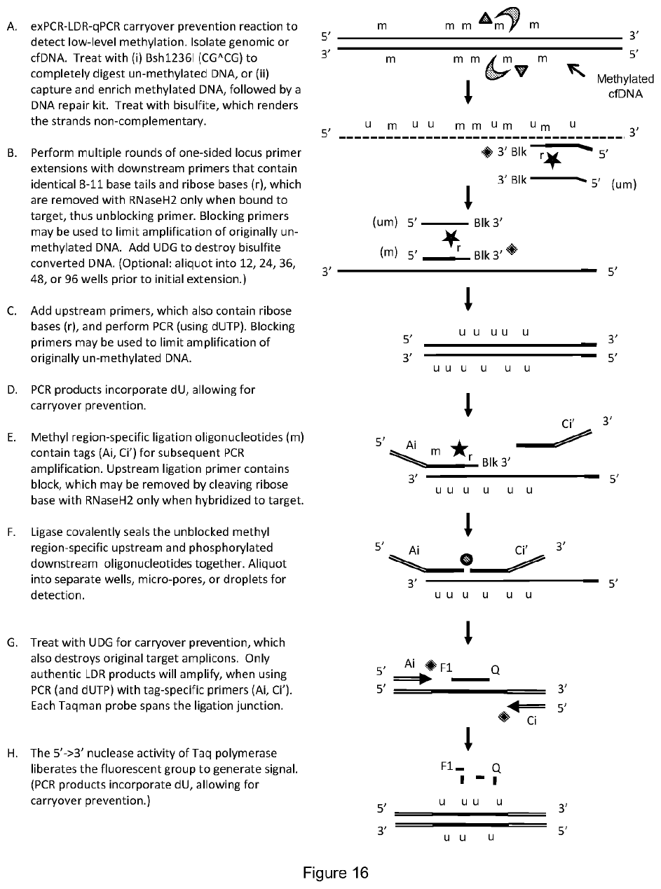

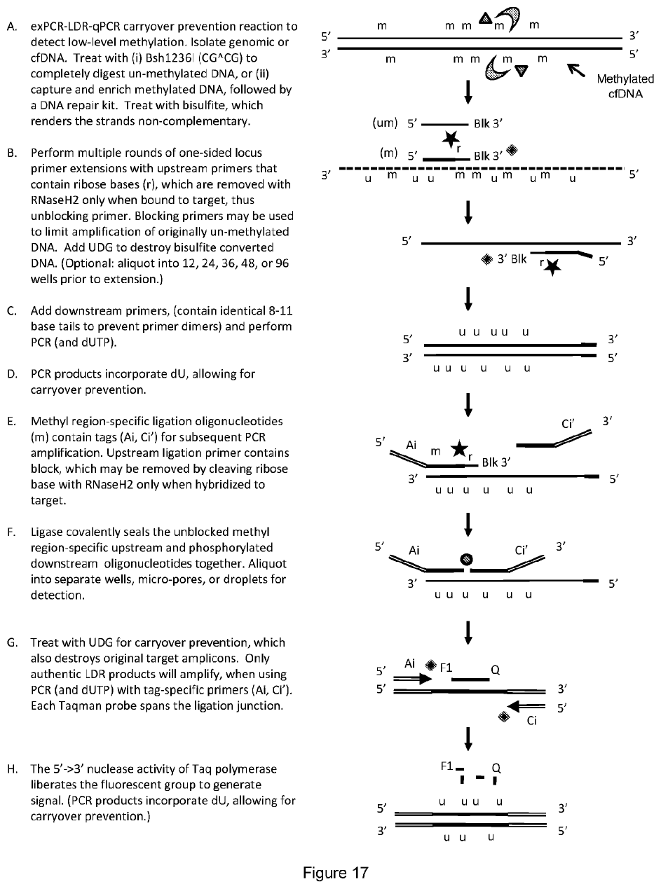

FIGS. 16 and 17 illustrate additional exemplary exPCR-LDR-qPCR carryover prevention reactions to detect low-level methylation. Genomic or cfDNA is isolated and then either treated with: (i) methyl-sensitive restriction endonucleases, e.g., Bsh1236I (CG\hat{ }CG), to completely digest unmethylated DNA and prevent carryover, or (ii) capture and enrich for methylated DNA, (iii) followed by a DNA repair kit (FIGS. 16 and 17, step A). The DNA is bisulfite-treated to convert unmethylated residues to uracil thereby rendering the double stranded DNA non-complementary. The regions of interest are selectively extended using locus-specific downstream primers (with optional identical 8-11 base tails), and a deoxynucleotide mix that does NOT include dUTP. In this embodiment, another layer of selectivity can be incorporated into the method by including a 3′ cleavable blocking group (Blk 3′, e.g. C3 spacer), and an RNA base (r), in the downstream primer. Upon target-specific hybridization, RNase H (star symbol) removes the RNA base to liberate a 3′OH group which is suitable for polymerase extension (FIG. 16, step B). If the locus-specific downstream primer covers one or more methylation sites, another layer of specificity may be added by using blocking primers whose sequence corresponds to the bisulfite-converted unmethylated sequence. After the extension cycles, add UDG, which destroys the bisulfite converted DNA (but not the primer extension products). Optionally aliquot sample into 12, 24, 36, 48, or 96 wells prior to the initial extension step. Subsequently, the regions of interest are selectively amplified in a limited cycle PCR (8-20 cycles) using locus-specific upstream primers, and a deoxynucleotide mix that does not include dUTP. In this embodiment, another layer of selectivity can be incorporated into the method by including a 3′ cleavable blocking group (Blk 3′, e.g. C3 spacer), and an RNA base (r), in the upstream primer. Upon target-specific hybridization, RNase H (star symbol) removes the RNA base to liberate a 3′OH group which is a few bases upstream of the bisulfite converted methylated target base, and suitable for polymerase extension (FIG. 16, step C). If the locus-specific upstream primer covers one or more methylation sites, another layer of specificity may be added by using blocking primers whose sequence corresponds to the bisulfite-converted unmethylated sequence.

Alternatively, as shown in FIG. 17, the regions of interest are selectively extended using locus-specific upstream primers for bisulfite converted DNA, and a deoxynucleotide mix that does not include dUTP. In this embodiment, another layer of selectivity can be incorporated into the method by including a 3′ cleavable blocking group (Blk 3′, e.g. C3 spacer), and an RNA base (r), in the upstream primer. Upon target-specific hybridization, RNase H (star symbol) removes the RNA base to liberate a 3′OH group which is a few bases upstream of the bisulfite converted methylated target base, and suitable for polymerase extension (FIG. 17, step B). If the locus-specific upstream primer covers one or more methylation sites, another layer of specificity may be added by using blocking primers whose sequence corresponds to the bisulfite-converted unmethylated sequence. Add UDG, which destroys the bisulfite converted DNA (but not the primer extension products). Subsequently, the locus-specific downstream primers are added, followed by limited (8 to 20 cycles) or full (20-40 cycles) PCR using a deoxynucleotide mix that includes dUTP (FIG. 17, step C). Optionally, the downstream primers contain identical 8-11 base tails to prevent primer dimers. Optionally aliquot sample into 12, 24, 36, 48, or 96 wells prior to the initial extension step. The amplified products contain dU as shown in FIG. 17, step D, which allows for subsequent treatment with UDG or a similar enzyme for carryover prevention.

For FIG. 16, methylation-specific upstream and locus-specific downstream probes containing tails (Ai, Ci′) enable formation of a ligation product in the presence of bisulfite converted methylated base-containing PCR products. Following ligation, the ligation products can be detected using pairs of matched primers Ai and Ci, and TaqMan™ probes that span the ligation junction as described supra for FIG. 2 (see FIG. 16, steps E-H), or using other suitable means known in the art.

Alternatively, methylation-specific upstream and locus-specific downstream probes containing tails (Ai, Bi′-Ci′) enable formation of a ligation product in the presence of bisulfite converted methylated base-containing PCR products. Following ligation, the ligation products are amplified using UniTaq-specific primers (i.e., F1-Bi-Q-Ai, Ci) and detected as described supra for FIG. 3, or using other suitable means known in the art.

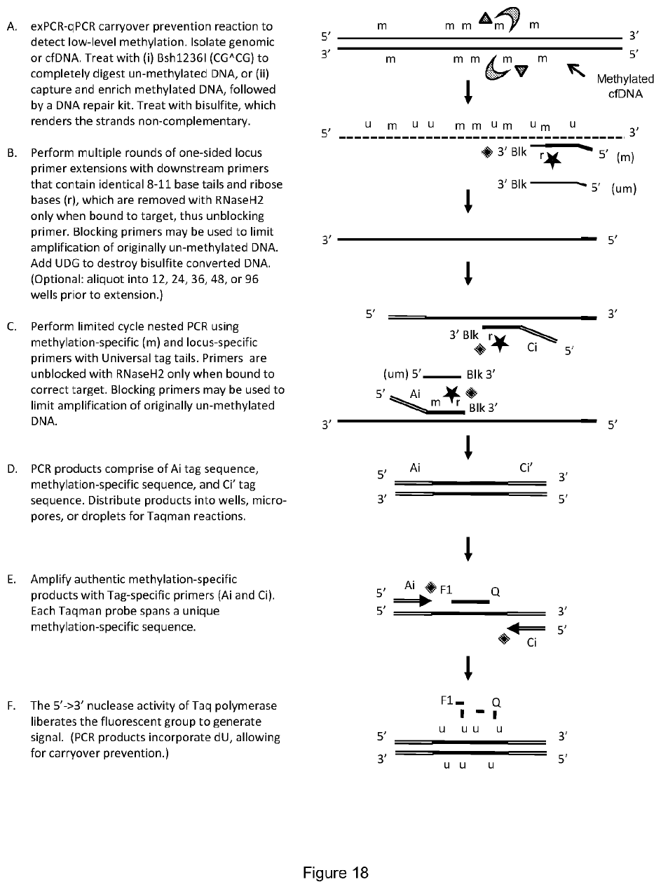

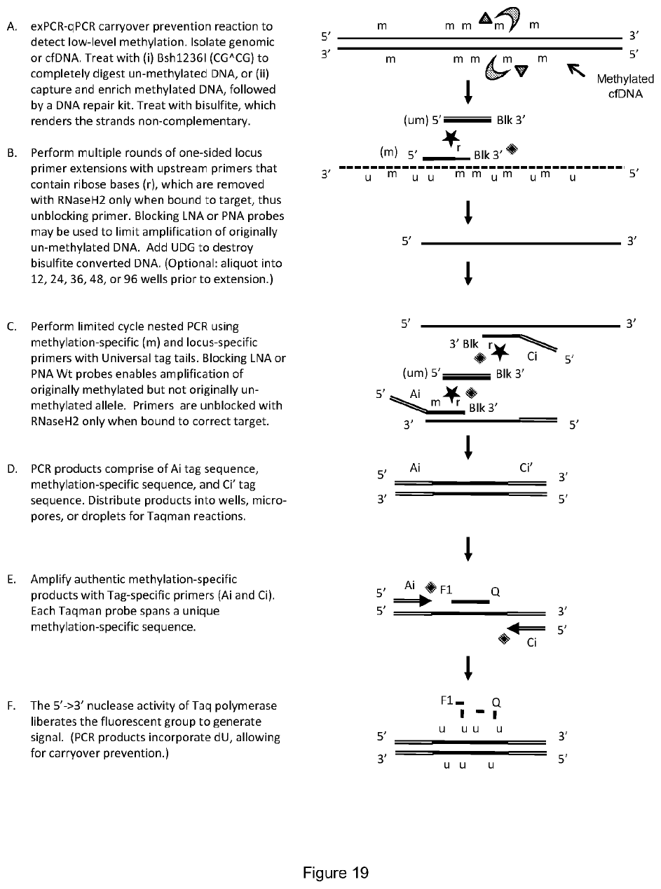

FIGS. 18 and 19 illustrate additional exemplary exPCR-LDR-qPCR carryover prevention reactions to detect low-level methylation. Genomic or cfDNA is isolated, and then either treated with: (i) methyl-sensitive restriction endonucleases, e.g., Bsh1236I (CG\hat{ }CG), to completely digest unmethylated DNA and prevent carryover, or (ii) capture and enrich for methylated DNA, (iii) followed by a DNA repair kit (FIGS. 18 and 19, step A). The DNA is bisulfite-treated to convert unmethylated residues to uracil thereby rendering the double stranded DNA non-complementary. The regions of interest are selectively extended using locus-specific downstream primers (with optional identical 8-11 base tails), and a deoxynucleotide mix that does NOT include dUTP. In this embodiment, another layer of selectivity can be incorporated into the method by including a 3′ cleavable blocking group (Blk 3′, e.g. C3 spacer), and an RNA base (r), in the downstream primer. Upon target-specific hybridization, RNase H (star symbol) removes the RNA base to liberate a 3′OH group which is suitable for polymerase extension (FIG. 18, step B). If the locus-specific downstream primer covers one or more methylation sites, another layer of specificity may be added by using blocking primers whose sequence corresponds to the bisulfite-converted unmethylated sequence. After the extension cycles, add UDG, which destroys the bisulfite converted DNA (but not the primer extension products). Optionally aliquot sample into 12, 24, 36, 48, or 96 wells prior to the initial extension step.

Alternatively, as shown in FIG. 19, the regions of interest are selectively extended using locus-specific upstream primers for bisulfite converted DNA, and a deoxynucleotide mix that does not include dUTP. Blocking LNA or PNA probes comprising the bisulfite converted unmethylated sequence (or its complement) enables amplification of originally methylated but not originally un-methylated alleles. In this embodiment, another layer of selectivity can be incorporated into the method by including a 3′ cleavable blocking group (Blk 3′, e.g. C3 spacer), and an RNA base (r), in the upstream primer. Upon target-specific hybridization, RNase H (star symbol) removes the RNA base to liberate a 3′OH group which is a few bases upstream of the bisulfite converted methylated target base, and suitable for polymerase extension (FIG. 19, step B). Add UDG, which destroys the bisulfite converted DNA (but not the primer extension products). Optionally aliquot sample into 12, 24, 36, 48, or 96 wells prior to the initial extension step.

As shown in FIG. 18, step C, bisulfite converted methylation base-specific primers (comprising 5′ primer-specific portions Ai) and bisulfite converted locus-specific primers (comprising 5′ primer-specific portions Ci) are added to then perform limited cycle nested PCR to amplify the bisulfite converted methylation-containing sequence, if present in the sample. Primers are unblocked with RNaseH2 only when bound to correct target. Following PCR, the products can be detected using pairs of matched primers Ai and Ci, and TaqMan™ probes that span the bisulfite-converted methylation target regions as described supra for FIG. 4 (see FIG. 18, steps D-F), or using other suitable means known in the art.

Alternatively, bisulfite converted methylation base-specific primers (comprising 5′ primer-specific portions Ai) and bisulfite converted locus-specific primers (comprising 5′ primer-specific portions Bi-Ci) are added to then perform limited cycle nested PCR to amplify the bisulfite converted methylation-containing sequence, if present in the sample. Primers are unblocked with RNaseH2 only when bound to correct target. Following PCR, the products are amplified using UniTaq-specific primers (i.e., F1-Bi-Q-Ai, Ci) and detected as described supra for FIG. 5, or using other suitable means known in the art.

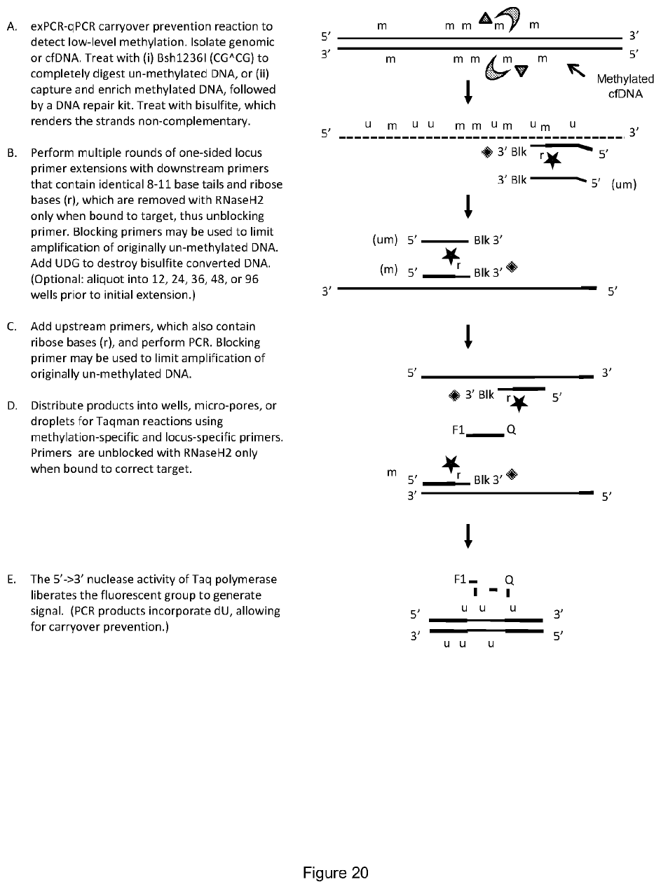

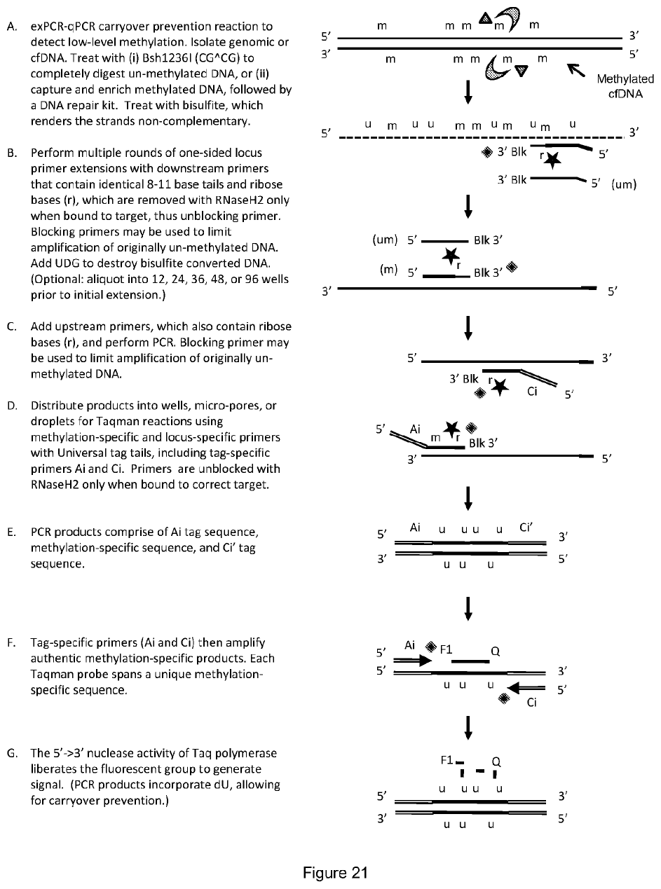

FIG. 20 illustrates another exemplary exPCR-qPCR carryover prevention reaction to detect low-level methylation. Genomic or cfDNA is isolated and then either treated with: (i) methyl-sensitive restriction endonucleases, e.g., Bsh12361 (CG\hat{ }CG), to completely digest unmethylated DNA and prevent carryover, or (ii) capture and enrich for methylated DNA, (iii) followed by a DNA repair kit (FIG. 20, step A). The DNA is bisulfite-treated to convert unmethylated residues to uracil thereby rendering the double stranded DNA non-complementary. The regions of interest are selectively extended using locus-specific downstream primers (with optional identical 8-11 base tails), and a deoxynucleotide mix that does not include dUTP. In this embodiment, another layer of selectivity can be incorporated into the method by including a 3′ cleavable blocking group (Blk 3′, e.g. C3 spacer), and an RNA base (r), in the downstream primer. Upon target-specific hybridization, RNase H (star symbol) removes the RNA base to liberate a 3′OH group which is suitable for polymerase extension (FIG. 20, step B). If the locus-specific downstream primer covers one or more methylation sites, another layer of specificity may be added by using blocking primers whose sequence corresponds to the bisulfite-converted unmethylated sequence. After the extension cycles, add UDG, which destroys the bisulfite converted DNA (but not the primer extension products). Optionally aliquot sample into 12, 24, 36, 48, or 96 wells prior to the initial extension step. Subsequently, the regions of interest are selectively amplified in a limited cycle PCR (8-20 cycles) using locus-specific upstream primers, and a deoxynucleotide mix that does not include dUTP. In this embodiment, another layer of selectivity can be incorporated into the method by including a 3′ cleavable blocking group (Blk 3′, e.g. C3 spacer), and an RNA base (r), in the upstream primer. Upon target-specific hybridization, RNase H (star symbol) removes the RNA base to liberate a 3′OH group which is a few bases upstream of the bisulfite converted methylated target base, and suitable for polymerase extension (FIG. 20, step C). If the locus-specific upstream primer covers one or more methylation sites, another layer of specificity may be added by using blocking primers whose sequence corresponds to the bisulfite-converted unmethylated sequence.

Following the limited cycle PCR, the PCR products are aliquoted into separate wells, micro-pores or droplets containing Taqman™ probes, bisulfite-converted, methylation base-specific, and bisulfite converted locus-specific primers, to amplify the bisulfite converted methylation-containing sequence, if present in the sample (FIG. 20, step D). The bisulfite converted methylation-containing products are amplified and detected as described supra for FIG. 6 (see FIG. 20, steps D-E), or using other suitable means known in the art.

FIG. 21 illustrate additional exemplary exPCR-qPCR carryover prevention reaction to detect low-level methylation. Genomic or cfDNA is isolated, and then either treated with: (i) methyl-sensitive restriction endonucleases, e.g., Bsh1236I (CG\hat{ }CG), to completely digest unmethylated DNA and prevent carryover, or (ii) capture and enrich for methylated DNA, (iii) followed by a DNA repair kit (FIG. 21, step A). The DNA is bisulfite-treated to convert unmethylated residues to uracil thereby rendering the double stranded DNA non-complementary. The regions of interest are selectively extended using locus-specific downstream primers (with optional identical 8-11 base tails), and a deoxynucleotide mix that does not include dUTP. In this embodiment, another layer of selectivity can be incorporated into the method by including a 3′ cleavable blocking group (Blk 3′, e.g. C3 spacer), and an RNA base (r), in the downstream primer. Upon target-specific hybridization, RNase H (star symbol) removes the RNA base to liberate a 3′OH group which is suitable for polymerase extension (FIG. 21, step B). If the locus-specific downstream primer covers one or more methylation sites, another layer of specificity may be added by using blocking primers whose sequence corresponds to the bisulfite-converted unmethylated sequence. After the extension cycles, UDG, which destroys the bisulfite converted DNA (but not the primer extension products) is added. Optionally, samples are aliquoted into 12, 24, 36, 48, or 96 wells prior to the initial extension step. Subsequently, the regions of interest are selectively amplified in a limited cycle PCR (8-20 cycles) using locus-specific upstream primers, and a deoxynucleotide mix that does not include dUTP. In this embodiment, another layer of selectivity can be incorporated into the method by including a 3′ cleavable blocking group (Blk 3′, e.g. C3 spacer), and an RNA base (r), in the upstream primer. Upon target-specific hybridization, RNase H (star symbol) removes the RNA base to liberate a 3′OH group which is a few bases upstream of the bisulfite converted methylated target base, and suitable for polymerase extension (FIG. 21, step C). If the locus-specific upstream primer covers one or more methylation sites, another layer of specificity may be added by using blocking primers whose sequence corresponds to the bisulfite-converted unmethylated sequence.

For the protocol illustrated in FIG. 21, following the limited cycle PCR, the PCR products are aliquoted into separate wells, micro-pores or droplets containing Taqman™ probes, bisulfite converted methylation base-specific primers comprising 5′ primer-specific portions (Ai), bisulfite converted locus-specific primers comprising 5′ primer-specific portions (Ci) and matching primers Ai and Ci. These primers combine to amplify the bisulfite converted methylation-containing sequence, if present in the sample (FIG. 21, step D). Primers are unblocked with RNaseH2 only when bound to correct target. Following PCR, the products can be detected using pairs of matched primers Ai and Ci, and TaqMan™ probes that span the bisulfite-converted methylation target regions as described supra for FIG. 4 (see FIG. 21, steps E-G), or using other suitable means known in the art.

Alternatively, following the limited cycle PCR, the PCR products are aliquoted into separate wells, micro-pores or droplets containing Taqman™ probes, bisulfite converted methylation base-specific primers comprising 5′ primer-specific portions (Ai), bisulfite converted locus-specific primers comprising 5′ primer-specific portions (Bi-Ci) and matching UniTaq primers F1-Bi-Q-Ai and Ci. Primers are unblocked with RNaseH2 only when bound to correct target. Following PCR, the products are amplified using UniTaq-specific primers (i.e., F1-Bi-Q-Ai, Ci) and detected as described supra for FIG. 5, or using other suitable means known in the art.

Another aspect of the present application is directed to a method for identifying, in a sample, one or more parent nucleic acid molecules containing a target nucleotide sequence differing from nucleotide sequences in other parent nucleic acid molecules in the sample by one or more methylated residues. The method involves providing a sample containing one or more parent nucleic acid molecules potentially containing the target nucleotide sequence differing from the nucleotide sequences in other parent nucleic acid molecules by one or more methylated residues, and subjecting the nucleic acid molecules in the sample to a bisulfite treatment under conditions suitable to convert unmethylated cytosine residues to uracil residues. One or more enzymes capable of digesting deoxyuracil (dU)-containing nucleic acid molecules present in the sample are provided. One or more primary oligonucleotide primer sets are also provided. Each primary oligonucleotide primer set comprises (a) a first primary oligonucleotide primer having a 5′ primer-specific portion and a 3′ portion that comprises a nucleotide sequence that is complementary to a sequence in the bisulfite-treated parent nucleic acid molecules adjacent to the bisulfite-treated target nucleotide sequence containing the one or more methylated residue and (b) a second primary oligonucleotide primer having a 5′ primer-specific portion and a 3′ portion that comprises a nucleotide sequence that is complementary to a portion of an extension product formed from the first primary oligonucleotide primer. The bisulfite treated sample, the one or more first primary oligonucleotide primers of the one or more primary oligonucleotide primer sets, a deoxynucleotide mix, and a DNA polymerase are blended to form one or more polymerase extension reaction mixtures. The one or more polymerase extension reaction mixtures are subjected to conditions suitable for one or more polymerase extension reaction cycles comprising a denaturation treatment, a hybridization treatment, and an extension treatment, thereby forming primary extension products comprising the complement of the bisulfite treated target nucleotide sequence. The one or more polymerase extension reaction mixtures comprising the primary extension products, the one or more secondary primary oligonucleotide primers of the one or more primary oligonucleotide primer sets, the one or more enzymes capable of digesting deoxyuracil (dU)-containing nucleic acid molecules in the reaction mixture, a deoxynucleotide mix, and a DNA polymerase are blended to form one or more first polymerase chain reaction mixtures. The method further comprises subjecting the one or more first polymerase chain reaction mixtures to conditions suitable for digesting deoxyuracil (dU)-containing nucleic acid molecules present in the polymerase chain reaction mixtures and for carrying out one or more polymerase chain reaction cycles comprising a denaturation treatment, a hybridization treatment, and an extension treatment, thereby forming first polymerase chain reactions products comprising the bisulfite-treated target nucleotide sequence or a complement thereof. One or more secondary oligonucleotide primer sets are provided. Each secondary oligonucleotide primer set comprises (a) a first secondary oligonucleotide primer comprising the same nucleotide sequence as the 5′ primer-specific portion of the first polymerase chain reaction products or their complements and (b) a second secondary oligonucleotide primer comprising a nucleotide sequence that is complementary to the 3′ primer-specific portion of the first polymerase chain reaction products or their complements. The primary polymerase chain reaction product sequences, the one or more secondary oligonucleotide primer sets, the one or more enzymes capable of digesting deoxyuracil (dU)-containing nucleic acid molecules, a deoxynucleotide mix including dUTP, and a DNA polymerase are blended to form one or more second polymerase chain reaction mixtures. The one or more second polymerase chain reaction mixtures are subjected to conditions suitable for digesting deoxyuracil (dU)-containing nucleic acid molecules present in the second polymerase chain reaction mixtures and for carrying out one or more polymerase chain reaction cycles comprising a denaturation treatment, a hybridization treatment, and an extension treatment thereby forming second polymerase chain reaction products. The method further involves detecting and distinguishing the second polymerase chain reactions products in the one or more second polymerase chain reaction mixtures to identify the presence of one or more parent nucleic acid molecules containing target nucleotide sequences differing from nucleotide sequences in other parent nucleic acid molecules in the sample by one or more methylated residues.

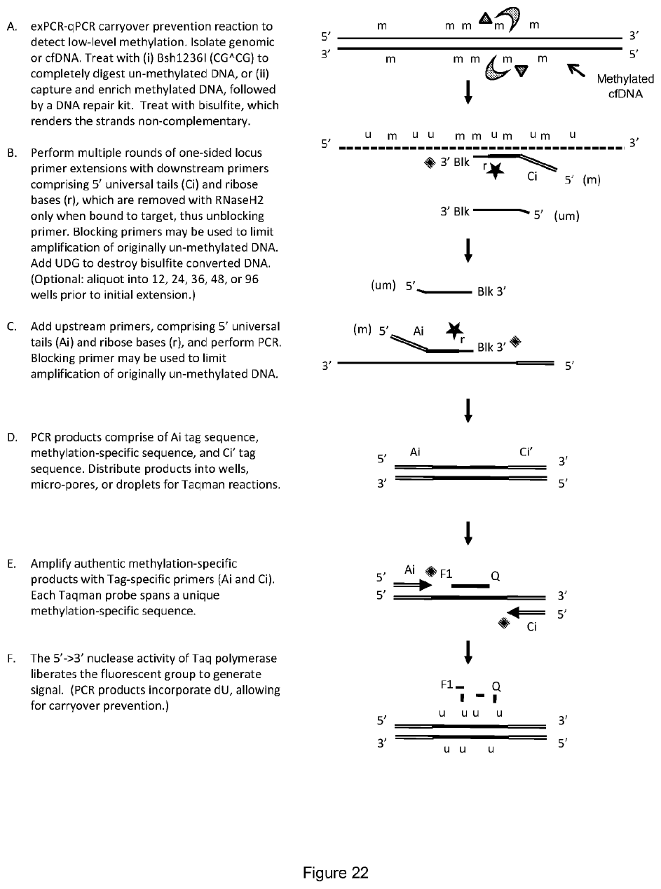

FIG. 22 illustrates an embodiment of this aspect of the present application.

FIG. 22 illustrates an additional exemplary exPCR-qPCR carryover prevention reaction to detect low-level methylation. Genomic or cfDNA is isolated, and then either treated with: (i) methyl-sensitive restriction endonucleases, e.g., Bsh12361 (CG\hat{ }CG), to completely digest unmethylated DNA and prevent carryover, or (ii) capture and enrich for methylated DNA, (iii) followed by a DNA repair kit (FIG. 22, step A). The DNA is bisulfite-treated to convert unmethylated residues to uracil thereby rendering the double stranded DNA non-complementary. The regions of interest are selectively extended using bisulfite converted locus-specific downstream primers comprising 5′ primer-specific portions (Ci for FIG. 22), and a deoxynucleotide mix that does not include dUTP. In this embodiment, another layer of selectivity can be incorporated into the method by including a 3′ cleavable blocking group (Blk 3′, e.g. C3 spacer), and an RNA base (r), in the downstream primer. Upon target-specific hybridization, RNase H (star symbol) removes the RNA base to liberate a 3′OH group which is suitable for polymerase extension (FIG. 22, step B). In this embodiment, the locus-specific downstream primer covers one or more methylation sites, and another layer of specificity may be added by using blocking primers whose sequence corresponds to the bisulfite-converted unmethylated sequence. After the extension cycles, add UDG, which destroys the bisulfite converted DNA (but not the primer extension products). Optionally aliquot sample into 12, 24, 36, 48, or 96 wells prior to the initial extension step. Subsequently, the regions of interest are selectively amplified in a limited cycle PCR (8-20 cycles) using bisulfite converted methylation base-specific upstream primers comprising 5′ primer-specific portions (Ai), and a deoxynucleotide mix that does not include dUTP. In this embodiment, another layer of selectivity can be incorporated into the method by including a 3′ cleavable blocking group (Blk 3′, e.g. C3 spacer), and an RNA base (r), in the upstream primer. Upon target-specific hybridization, RNase H (star symbol) removes the RNA base to liberate a 3′OH group which is a few bases upstream of the bisulfite converted methylated target base, and suitable for polymerase extension (FIG. 22, step C). Since the methylation base-specific upstream primer covers one or more methylation sites, another layer of specificity may be added by using blocking primers whose sequence corresponds to the bisulfite-converted unmethylated sequence.

As shown in FIG. 22 step D, the limited cycle PCR products comprise of Ai tag sequence, methylation-specific sequence, and Ci′ tag sequence, and are distributed into wells, micro-pores, or droplets for Taqman™ reactions. Following PCR, the products can be detected using pairs of matched primers Ai and Ci, and TaqMan™ probes that span the bisulfite-converted methylation target regions as described supra for FIG. 4 (see FIG. 22, steps D-F), or using other suitable means known in the art.

Alternatively, the limited cycle PCR products comprise of Ai tag sequence, methylation-specific sequence, and Bi′-Ci′ tag sequence, and are distributed into wells, micro-pores, or droplets for Taqman™ reactions. Following PCR, the products are amplified using UniTaq-specific primers (i.e., F1-Bi-Q-Ai, Ci) and detected as described supra for FIG. 5, or using other suitable means known in the art.

In one embodiment, the method further comprises contacting the sample with DNA repair enzymes to repair damaged DNA, abasic sites, oxidized bases, or nicks in the DNA.

In another embodiment, the method further comprises contacting the sample with at least a first methylation sensitive enzyme to form a restriction enzyme reaction mixture prior to, or concurrent with, said blending to form one or more polymerase extension reaction mixtures, wherein said first methylation sensitive enzyme cleaves nucleic acid molecules in the sample that contain one or more unmethylated residues within at least one methylation sensitive enzyme recognition sequence, and whereby said detecting involves detection of one or more parent nucleic acid molecules containing the target nucleotide sequence, wherein said parent nucleic acid molecules originally contained one or more methylated residues.

In accordance with this and all aspects of the present invention, a “methylation sensitive enzyme” is an endonuclease that will not cleave or has reduced cleavage efficiency of its cognate recognition sequence in a nucleic acid molecule when the recognition sequence contains a methylated residue (i.e., it is sensitive to the presence of a methylated residue within its recognition sequence). A “methylation sensitive enzyme recognition sequence” is the cognate recognition sequence for a methylation sensitive enzyme. In some embodiments, the methylated residue is a 5-methyl-C, within the sequence CpG (i.e., 5-methyl-CpG). A non-limiting list of methylation sensitive restriction endonuclease enzymes that are suitable for use in the methods of the present invention include, without limitation, AciI, HinP1I, Hpy99I, HpyCH4IV, BstUI, HpaII, HhaI, or any combination thereof.

In a further embodiment, the method further comprises contacting the sample with an immobilized methylated nucleic acid binding protein or antibody to selectively bind and enrich for methylated nucleic acid in the sample.

In one embodiment, the primers from the one or more primary or secondary oligonucleotide primer sets comprise a portion that has no or one nucleotide sequence mismatch when hybridized in a base-specific manner to the target nucleic acid sequence or bisulfite-converted methylated nucleic acid sequence or complement sequence thereof, but have one or more additional nucleotide sequence mismatches that interferes with polymerase extension when primers from said one or more primary or secondary oligonucleotide primer sets hybridize in a base-specific manner to a corresponding nucleotide sequence portion in wildtype nucleic acid sequence or bisulfate-converted unmethylated nucleic acid sequence or complement sequence thereof.

One or both primary oligonucleotide primers of the primary oligonucleotide primer set and/or one or both secondary oligonucleotide primers of the secondary oligonucleotide primer set may have a 3′ portion comprising a cleavable nucleotide or nucleotide analogue and a blocking group, such that the 3′ end of said primer or primers is unsuitable for polymerase extension. This embodiment of the method further comprises cleaving the cleavable nucleotide or nucleotide analog of one or both oligonucleotide primers during said hybridization treatment, thereby liberating free 3′OH ends on one or both oligonucleotide primers prior to said extension treatment. In one embodiment, the cleavable nucleotide comprises one or more RNA bases.

In another embodiment, primers from the one or more primary or secondary oligonucleotide primer sets comprise a sequence that differs from the target nucleic acid sequence or bisulfite-converted methylated nucleic acid sequence or complement sequence thereof, said difference is located two or three nucleotide bases from the liberated free 3′OH end.

In another embodiment, the method further comprises providing one or more blocking oligonucleotide primers comprising one or more mismatched bases at the 3′ end or one or more nucleotide analogs and a blocking group at the 3′ end, such that the 3′ end of said blocking oligonucleotide primer is unsuitable for polymerase extension when hybridized in a base-specific manner to wildtype nucleic acid sequence or bisulfite-converted unmethylated nucleic acid sequence or complement sequence thereof, wherein said blocking oligonucleotide primer comprises a portion having a nucleotide sequence that is the same as a nucleotide sequence portion in the wildtype nucleic acid sequence or bisulfite-converted unmethylated nucleic acid sequence or complement sequence thereof to which the blocking oligonucleotide primer hybridizes but has one or more nucleotide sequence mismatches to a corresponding nucleotide sequence portion in the target nucleic acid sequence or bisulfate-converted methylated nucleic acid sequence or complement sequence thereof. The one or more blocking oligonucleotide primers are blended with the sample or subsequent products prior to a polymerase extension reaction, polymerase chain reaction, or ligation reaction, whereby during hybridization the one or more blocking oligonucleotide primers preferentially hybridize in a base-specific manner to a wildtype nucleic acid sequence or bisulfite-converted unmethylated nucleic acid sequence or complement sequence thereof, thereby interfering with polymerase extension or ligation during reaction of a primer or probes hybridized in a base-specific manner to the wildtype sequence or bisulfite-converted unmethylated sequence or complement sequence thereof.

In certain embodiments, the first secondary oligonucleotide primer has a 5′ primer-specific portion and the second secondary oligonucleotide primer has a 5′ primer-specific portion, said one or more secondary oligonucleotide primer sets further comprising a third secondary oligonucleotide primer comprising the same nucleotide sequence as the 5′ primer-specific portion of the first secondary oligonucleotide primer and (d) a fourth secondary oligonucleotide primer comprising the same nucleotide sequence as the 5′ primer-specific portion of the second secondary oligonucleotide primer.

Another aspect of the present application is directed to a method for identifying in a sample, one or more parent ribonucleic acid molecules containing a target ribonucleic acid sequence differing from ribonucleic acid sequences of other parent ribonucleic acid molecules in the sample due to alternative splicing, alternative transcript, alternative start site, alternative coding sequence, alternative non-coding sequence, exon insertion, exon deletion, intron insertion, translocation, mutation, or other rearrangement at the genome level. The method involves providing a sample containing one or more parent ribonucleic acid molecules containing a target ribonucleic acid molecule potentially differing in sequence from other parent ribonucleic acid molecules and providing one or more enzymes capable of digesting deoxyuracil (dU) containing nucleic acid molecules present in the sample. The sample is contacted with one or more enzymes capable of digesting dU containing nucleic acid molecules potentially present in the sample. One or more primary oligonucleotide primer sets are then provided. Each primary oligonucleotide primer set comprises (a) a first primary oligonucleotide primer that comprises a nucleotide sequence that is complementary to the RNA sequence in the parent ribonucleic acid molecule adjacent to the target ribonucleotide sequence and (b) a second primary oligonucleotide primer that comprises a nucleotide sequence that is complementary to a portion of the cDNA extension product formed from the first primary oligonucleotide primer. The contacted sample, the one or more primary oligonucleotide primer sets, a deoxynucleotide mix including dUTP, a reverse transcriptase, and a DNA polymerase or a DNA polymerase with reverse-transcriptase activity are blended to form one or more reverse-transcription/polymerase chain reaction mixtures. The one or more reverse-transcription/polymerase chain reaction mixtures are subjected to conditions suitable for generating complementary deoxyribonucleic acid (cDNA) molecules to the target ribonucleic nucleic acid and to carry out one or more polymerase chain reaction cycles comprising a denaturation treatment, a hybridization treatment, and an extension treatment thereby forming one or more different reverse transcription/polymerase products. The method further comprises providing one or more oligonucleotide probe sets. Each probe set comprises (a) a first oligonucleotide probe having a 5′ primer-specific portion and a 3′ target sequence-specific portion, and (b) a second oligonucleotide probe having a 5′ target sequence-specific portion and a 3′ primer-specific portion, wherein the first and second oligonucleotide probes of a probe set are configured to hybridize, in a base specific manner, on complementary portions of a reverse transcriptase/polymerase product corresponding to the target ribonucleic acid molecule sequence. The reverse transcriptase/polymerase products are contacted with a ligase and the one or more oligonucleotide probe sets to form one or more ligation reaction mixtures, and the one or more ligation reaction mixtures are subjected to one or more ligation reaction cycles whereby the first and second probes of the one or more oligonucleotide probe sets, when hybridized to their complement, are ligated together to form ligated product sequences in the ligase reaction mixture, wherein each ligated product sequence comprises the 5′ primer-specific portion, the target-specific portions, and the 3′ primer-specific portion. The method further involves providing one or more secondary oligonucleotide primer sets. Each secondary oligonucleotide primer set comprises (a) a first secondary oligonucleotide primer comprising the same nucleotide sequence as the 5′ primer-specific portion of the ligated product sequence and (b) a second secondary oligonucleotide primer comprising a nucleotide sequence that is complementary to the 3′ primer-specific portion of the ligated product sequence. The ligated product sequences, the one or more secondary oligonucleotide primer sets with one or more enzymes capable of digesting deoxyuracil (dU)-containing nucleic acid molecules, a deoxynucleotide mix including dUTP, and a DNA polymerase are blended to form one or more first polymerase chain reaction mixtures, and the one or more first polymerase chain reaction mixtures are subjected to conditions suitable for digesting deoxyuracil (dU)-containing nucleic acid molecules present in the first polymerase chain reaction mixtures and for carrying out one or more polymerase chain reaction cycles comprising a denaturation treatment, a hybridization treatment, and an extension treatment thereby forming first polymerase chain reaction products. The method further comprises detecting and distinguishing the first polymerase chain reaction products, thereby identifying the presence of one or more parent ribonucleic acid molecules containing a target ribonucleic acid sequence differing from ribonucleic acid sequences of other parent ribonucleic acid molecules in the sample due to alternative splicing, alternative transcript, alternative start site, alternative coding sequence, alternative non-coding sequence, exon insertion, exon deletion, intron insertion, translocation, mutation, or other rearrangement at the genome level.

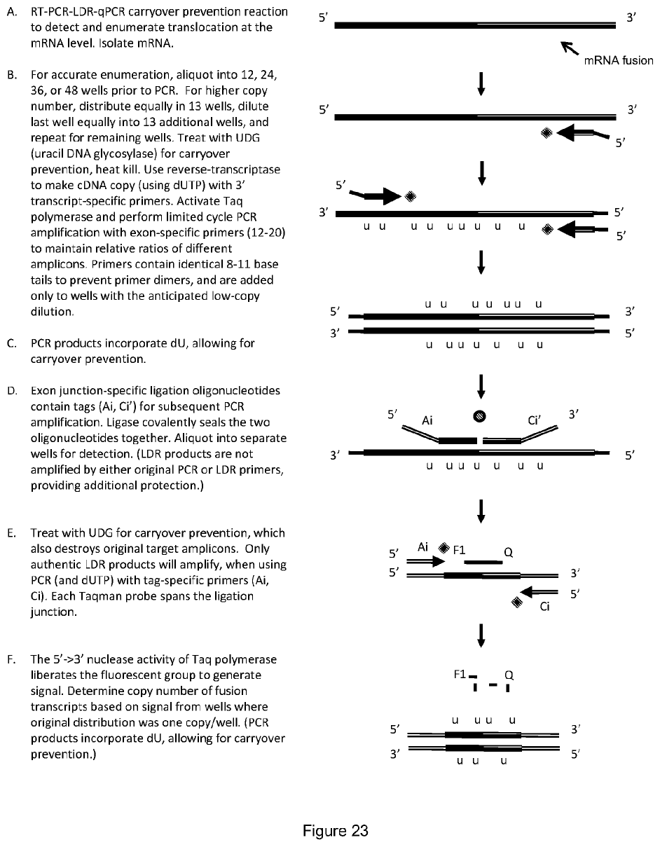

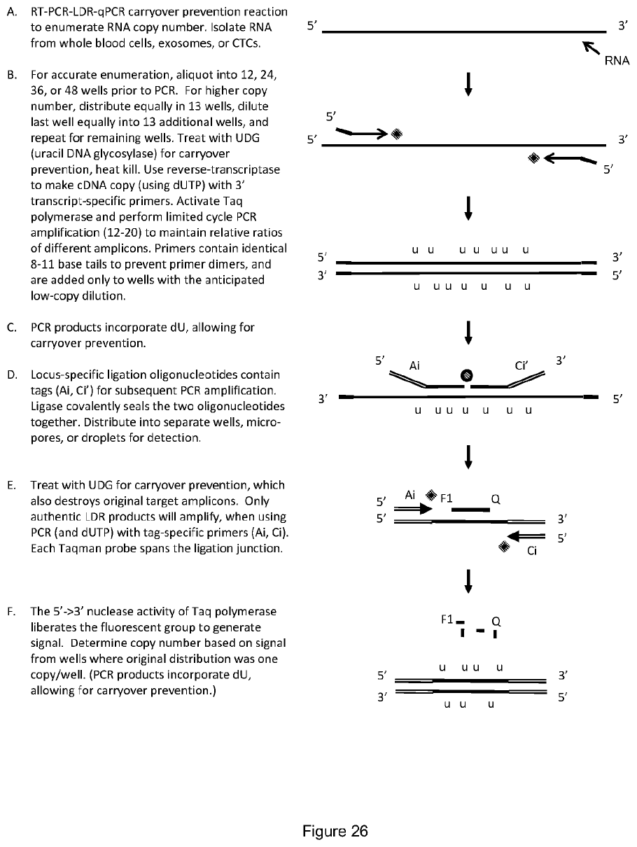

FIGS. 23 and 26 illustrate embodiments of this aspect of the present application.

FIG. 23 illustrates an exemplary RT-PCR-LDR-qPCR carryover prevention reaction to detect translocations at the mRNA level. Such fusion mRNA may be isolated from circulating tumor cells, exosomes or from other plasma fractions. For accurate enumeration, aliquot into 12, 24, 36, or 48 wells prior to PCR. For higher copy number, distribute equally in 13 wells, dilute last well equally into 13 additional wells, and repeat for remaining wells. In this embodiment, mRNA is isolated (FIG. 23, step A), and treated with UDG for carryover prevention (FIG. 23, step B). cDNA is generated using 3′ transcript-specific primers and reverse-transcriptase in the presence of dUTP. Suitable reverse transcriptases include but are not limited to Moloney Murine Leukemia Virus Reverse Transcriptase (M-MLV RT, New England Biolabs), or Superscript II or III Reverse Transcriptase (Life Technologies). Taq polymerase is activated to perform limited cycle PCR amplification (12-20) to maintain relative ratios of different amplicons (FIG. 23, step B). The primers contain identical 8-11 base tails to prevent primer dimers. PCR products incorporate dUTP, allowing for carryover prevention (FIG. 23, step C).

As shown in FIG. 23, step D, exon junction-specific ligation oligonucleotide probes containing primer-specific portions (Ai, Ci′) suitable for subsequent PCR amplification, hybridize to their corresponding target sequence in a base-specific manner. Ligase covalently seals the two oligonucleotides together (FIG. 23, step D), and ligation products are aliquoted into separate wells for detection using tag-primers (Ai, Ci) and TaqMan™ probe (F1-Q) which spans the ligation junction (FIG. 23, step E). Treat samples with UDG for carryover prevention, which also destroys original target amplicons (FIG. 23, step E). Only authentic LDR products will amplify, when using PCR in the presence of dUTP. Copy number of fusion transcripts is determined based on signal from wells where original distribution was one copy/well. Neither original PCR primers nor LDR probes amplify LDR products, providing additional carryover protection.

Another aspect of the present application is directed to a method for identifying in a sample, one or more parent ribonucleic acid molecules containing a target ribonucleic acid sequence differing from ribonucleic acid sequences of other parent ribonucleic acid molecules in the sample due to alternative splicing, alternative transcript, alternative start site, alternative coding sequence, alternative non-coding sequence, exon insertion, exon deletion, intron insertion, translocation, mutation, or other rearrangement at the genome level. The method involves providing a sample containing one or more parent ribonucleic acid molecules containing a target ribonucleic acid molecule potentially differing in sequence from other parent ribonucleic acid molecules and providing one or more enzymes capable of digesting deoxyuracil (dU)-containing nucleic acid molecules present in the sample. The sample is contacted with one or more enzymes capable of digesting dU containing nucleic acid molecules potentially present in the sample. The method further involves providing one or more primary oligonucleotide primer sets, each primary oligonucleotide primer set comprising (a) a first primary oligonucleotide primer that comprises a nucleotide sequence that is complementary to the RNA sequence in the parent ribonucleic acid molecule adjacent to the target nucleotide sequence and (b) a second primary oligonucleotide primer that comprises a nucleotide sequence that is complementary to a portion of the cDNA extension product formed from the first primary oligonucleotide primer. The contacted sample, the one or more primary oligonucleotide primer sets, a deoxynucleotide mix, a reverse transcriptase and a DNA polymerase or a DNA polymerase with reverse-transcriptase activity are blended to form one or more reverse-transcription/polymerase chain reaction mixtures, and the one or more reverse-transcription/polymerase chain reaction mixtures are subjected to conditions suitable for generating complementary deoxyribonucleic acid (cDNA) molecules to the target RNA and to carry out one or more polymerase chain reaction cycles comprising a denaturation treatment, a hybridization treatment, and an extension treatment thereby forming one or more different reverse-transcription/primary polymerase chain reaction products. The method further comprises providing one or more secondary oligonucleotide primer sets. Each secondary oligonucleotide primer set comprises (a) a first secondary oligonucleotide primer having a 3′ portion that is complementary to a portion of a reverse-transcription/primary polymerase chain reaction product formed from the first primary oligonucleotide primer and (b) a second secondary oligonucleotide primer having a 3′ portion that comprises a nucleotide sequence that is complementary to a portion of a reverse-transcription/primary polymerase chain reaction product formed from the first secondary oligonucleotide primer. The reverse-transcription/primary polymerase chain reaction products, the one or more secondary oligonucleotide primer sets, the one or more enzymes capable of digesting deoxyuracil (dU) containing nucleic acid molecules, a deoxynucleotide mix including dUTP, and a DNA polymerase are blended to form one or more first polymerase chain reaction mixtures, and the one or more first polymerase chain reaction mixtures are subjected to conditions suitable for digesting deoxyuracil (dU)-containing nucleic acid molecules present in the first polymerase chain reaction mixtures and for carrying out two or more polymerase chain reaction cycles comprising a denaturation treatment, a hybridization treatment, and an extension treatment thereby forming first polymerase chain reaction products. The method further involves detecting and distinguishing the first polymerase chain reaction products, thereby identifying the presence of one or more parent ribonucleic acid molecules containing a target ribonucleic acid sequences differing from ribonucleic acid sequences of other parent ribonucleic acid molecules in the sample due to alternative splicing, alternative transcript, alternative start site, alternative coding sequence, alternative non-coding sequence, exon insertion, exon deletion, intron insertion, translocation, mutation, or other rearrangement at the genome level.

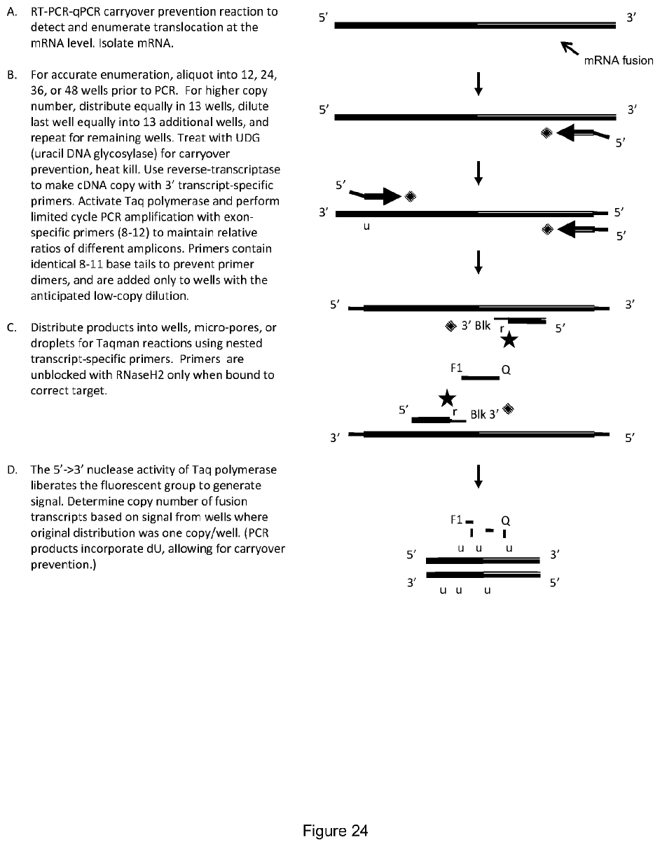

FIGS. 24, 25, 27, and 28 illustrate various embodiments of this aspect of the present application.

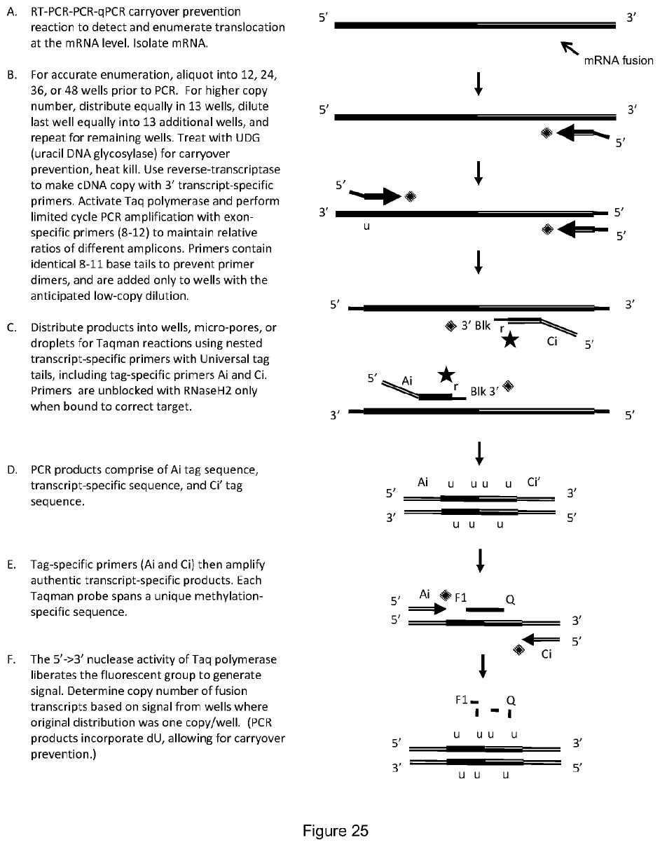

FIGS. 24 and 25 illustrate additional exemplary RT-PCR-LDR-qPCR carryover prevention reaction to detect translocations at the mRNA level. For accurate enumeration, aliquot into 12, 24, 36, or 48 wells prior to PCR. For higher copy number, distribute equally in 13 wells, dilute last well equally into 13 additional wells, and repeat for remaining wells. In this embodiment, mRNA is isolated (FIGS. 24 and 25, step A), and treated with UDG for carryover prevention (FIGS. 24 and 25, step B). cDNA is generated using 3′ transcript-specific primers and reverse-transcriptase. Taq polymerase is activated to perform limited cycle PCR amplification (8-20) to maintain relative ratios of different amplicons (FIGS. 24 and 25, step B). The primers contain identical 8-11 base tails to prevent primer dimers and are added only to wells with the anticipated low-copy dilution.

For the protocol illustrated in FIG. 24, following the limited cycle PCR, the PCR products are aliquoted into separate wells, micro-pores or droplets containing Taqman™ probes across the cDNA fusion junction, cDNA-specific primers, to amplify the junction sequence, if present in the sample (FIG. 24, step C). The fusion cDNA products are amplified and detected as described supra for FIG. 6 (see FIG. 24, steps C-D), or using other suitable means known in the art.

For the protocol illustrated in FIG. 25, following the limited cycle PCR, the PCR products are aliquoted into separate wells, micro-pores or droplets containing Taqman™ probes across the cDNA fusion junction, cDNA-specific (forward) primers comprising 5′ primer-specific portions (Ai), cDNA-specific (reverse) primers comprising 5′ primer-specific portions (Ci) and matching primers Ai and Ci. These primers combine to amplify the fusion cDNA sequence, if present in the sample (FIG. 25, step C). Primers are unblocked with RNaseH2 only when bound to correct target. Following PCR, the products can be detected using pairs of matched primers Ai and Ci, and TaqMan™ probes that span the fusion cDNA regions as described supra for FIG. 4 (see FIG. 25, steps D-F), or using other suitable means known in the art.

FIG. 26 illustrates an exemplary RT-PCR-LDR-qPCR carryover prevention reaction to enumerate mRNA, ncRNA, or lncRNA copy number. RNA is isolated from whole blood cells, exosomes, CTCs or other plasma fractions. For accurate enumeration, aliquot into 12, 24, 36, or 48 wells prior to PCR. For higher copy number, distribute equally in 13 wells, dilute last well equally into 13 additional wells, and repeat for remaining wells. In this embodiment, mRNA is isolated (FIG. 26, step A), and treated with UDG for carryover prevention (FIG. 26, step B). cDNA is generated using 3′ transcript-specific primers and reverse-transcriptase in the presence of dUTP. Taq polymerase is activated to perform limited cycle PCR amplification (12-20) to maintain relative ratios of different amplicons (FIG. 26, step B). The primers contain identical 8-11 base tails to prevent primer dimers. PCR products incorporate dUTP, allowing for carryover prevention (FIG. 26, step C).

As shown in FIG. 26, step D, exon junction-specific ligation oligonucleotide probes containing primer-specific portions (Ai, Ci′) suitable for subsequent PCR amplification, hybridize to their corresponding target sequence in a base-specific manner. Ligase covalently seals the two oligonucleotides together (FIG. 26, step D), and ligation products are aliquoted into separate wells for detection using tag-primers (Ai, Ci) and TaqMan™ probe (F1-Q) which spans the ligation junction (FIG. 26, step E). Treat samples with UDG for carryover prevention, which also destroys original target amplicons (FIG. 26, step E). Only authentic LDR products will amplify, when using PCR in presence of dUTP. Determine copy number of mRNA, ncRNA, or lncRNA transcripts based on signal from wells where original distribution was one copy/well. Neither original PCR primers nor LDR probes amplify LDR products, providing additional carryover protection.

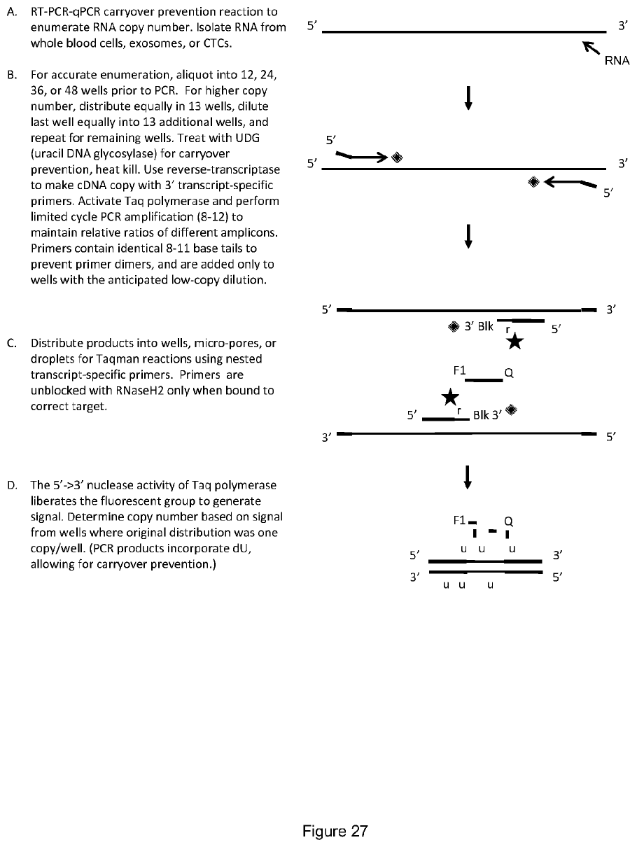

FIG. 27 illustrates an exemplary RT-PCR-LDR-qPCR carryover prevention reaction to enumerate mRNA, ncRNA, or lncRNA copy number. Isolate RNA from whole blood cells, exosomes, CTCs or other plasma fractions. For accurate enumeration, aliquot into 12, 24, 36, or 48 wells prior to PCR. For higher copy number, distribute equally in 13 wells, dilute last well equally into 13 additional wells, and repeat for remaining wells. In this embodiment, mRNA is isolated (FIG. 27, step A), and treated with UDG for carryover prevention (FIG. 27, step B). cDNA is generated using 3′ transcript-specific primers and reverse-transcriptase in the presence of dUTP. Taq polymerase is activated to perform limited cycle PCR amplification (8-20) to maintain relative ratios of different amplicons (FIG. 27, step B). The primers contain identical 8-11 base tails to prevent primer dimers. PCR products incorporate dUTP, allowing for carryover prevention (FIG. 27, step C).

For the protocol illustrated in FIG. 27, following the limited cycle PCR, the PCR products are aliquoted into separate wells, micro-pores or droplets containing Taqman™ probes across the cDNA target region, cDNA-specific primers, to amplify the target sequence, if present in the sample (FIG. 27, step C). The cDNA target products are amplified and detected as described supra for FIG. 6 (see FIG. 27, steps C-D), or using other suitable means known in the art.

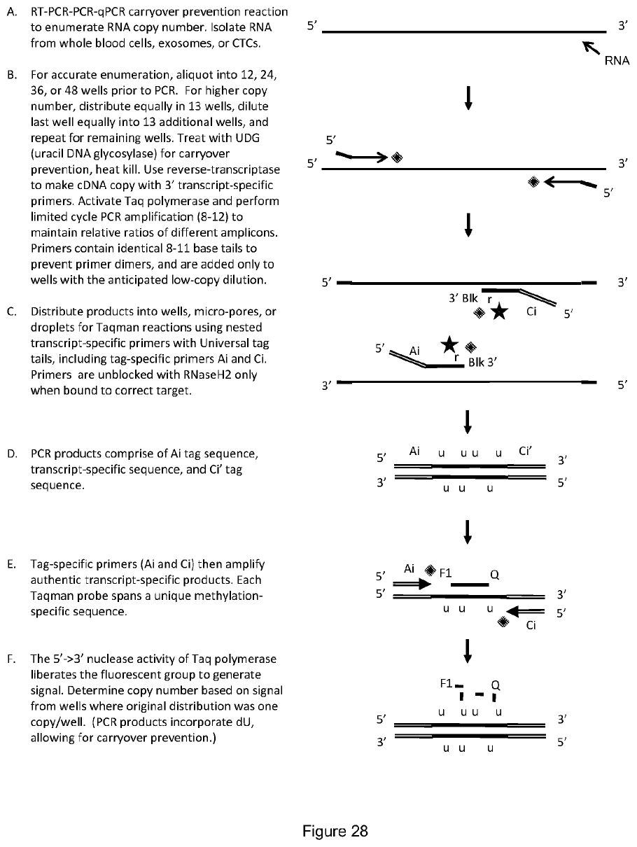

For the protocol illustrated in FIG. 28, following the limited cycle PCR, the PCR products are aliquoted into separate wells, micro-pores or droplets containing Taqman™ probes across the cDNA target region, cDNA-specific (forward) primers comprising 5′ primer-specific portions (Ai), cDNA-specific (reverse) primers comprising 5′ primer-specific portions (Ci) and matching primers Ai and Ci. These primers combine to amplify the target cDNA sequence, if present in the sample (FIG. 28, step C). Primers are unblocked with RNaseH2 only when bound to correct target. Following PCR, the products can be detected using pairs of matched primers Ai and Ci, and TaqMan™ probes that span the target cDNA regions as described supra for FIG. 4 (see FIG. 28, steps D-F), or using other suitable means known in the art.

Alternatively, following the limited cycle PCR, the PCR products are aliquoted into separate wells, micro-pores or droplets containing Taqman™ probes across the cDNA target region, cDNA-specific (forward) primers comprising 5′ primer-specific portions (Ai), cDNA-specific (reverse) primers comprising 5′ primer-specific portions (Bi-Ci) and matching primers F1-Bi-Q-Ai and Ci (Figure not shown). These primers combine to amplify the target cDNA sequence, if present in the sample. Primers are unblocked with RNaseH2 only when bound to correct target. Following PCR, the products can be detected using pairs of matched primers F1-Bi-Q-Ai and Ci, and TaqMan™ probes that span the target cDNA regions as described supra for FIG. 4, or using other suitable means known in the art.

Another aspect of the present application is directed to a method for identifying, in a sample, one or more target micro-ribonucleic acid (miRNA) molecules differing in sequence from other miRNA molecules in the sample by one or more bases. The method involves providing a sample containing one or more target miRNA molecules potentially differing in sequence from other miRNA molecules in the sample by one or more bases and providing one or more enzymes capable of digesting deoxyuracil (dU)-containing nucleic acid molecules present in the sample. The sample is contacted with one or more enzymes capable of digesting dU-containing nucleic acid molecules potentially present in the sample, and the contacted sample is blended with a ligase and one or more first oligonucleotide preliminary probes comprising a 5′ phosphate, a 5′ stem-loop portion, an internal primer-specific portion within the loop region, a blocking group, and a 3′ nucleotide sequence that is complementary to a 3′ portion of the target miRNA molecule sequence to form one or more first ligation reaction mixtures. The method further comprises ligating, in the one or more first ligation reaction mixtures, the one or more target miRNA molecules at their 3′ end to the 5′ phosphate of the one or more first oligonucleotide preliminary probes to generate chimeric nucleic acid molecules comprising the target miRNA molecule sequence, if present in the sample, appended to the one or more first oligonucleotide preliminary probes. One or more primary oligonucleotide primer sets are then provided. Each primer set comprises (a) a first primary oligonucleotide primer comprising a nucleotide sequence that is complementary to the internal primer-specific portion of the first oligonucleotide preliminary probe, and (b) a second primary oligonucleotide primer comprising a 5′ primer-specific portion and a 3′ portion, wherein the second primary oligonucleotide primer may be the same or may differ from other second primary oligonucleotide primers in other sets. The one or more first ligation reaction mixtures comprising chimeric nucleic acid molecules, the one or more primary oligonucleotide primer sets, the one or more enzymes capable of digesting deoxyuracil (dU)-containing nucleic acid molecules in the sample, a deoxynucleotide mix including dUTP, and a reverse transcriptase and a DNA polymerase or a DNA polymerase with reverse-transcriptase activity are blended to form one or more reverse-transcription/polymerase chain reaction mixtures. The one or more reverse-transcription/polymerase chain reaction mixtures are subjected to conditions suitable for digesting deoxyuracil (dU)-containing nucleic acid molecules present in the reverse-transcription/polymerase chain reaction mixtures to conditions suitable for generating complementary deoxyribonucleic acid (cDNA) molecules to the chimeric nucleic acid molecules, and to one or more polymerase chain reaction cycles comprising a denaturation treatment, a hybridization treatment, and an extension treatment thereby forming one or more different primary reverse-transcription/polymerase chain reaction products comprising the 5′ primer-specific portion, a nucleotide sequence corresponding to the target miRNA molecule sequence, and the complement of the internal primer-specific portion, and complements thereof. The method further involves providing one or more oligonucleotide probe sets. Each probe set comprises (a) a first oligonucleotide probe having a 5′ primer-specific portion and a 3′ target sequence-specific portion, and (b) a second oligonucleotide probe having a 5′ target sequence-specific portion, a portion complementary to a primary extension product, and a 3′ primer-specific portion, wherein the first and second oligonucleotide probes of a probe set are configured to hybridize, in a base specific manner, on complementary portions of a primary reverse-transcription/polymerase chain reaction product corresponding to the target miRNA molecule sequence, or complement thereof. The primary reverse-transcription/polymerase chain reaction products are contacted with a ligase and the one or more oligonucleotide probe sets to form one or more second ligation reaction mixtures, and the one or more second ligation reaction mixtures are subjected to one or more ligation reaction cycles whereby the first and second oligonucleotide probes of the one or more oligonucleotide probe sets, when hybridized to their complement, are ligated together to form ligated product sequences in the ligation reaction mixture, wherein each ligated product sequence comprises the 5′ primer-specific portion, the target-specific portions, and the 3′ primer-specific portion. The method further involves providing one or more secondary oligonucleotide primer sets. Each secondary oligonucleotide primer set comprises (a) a first secondary oligonucleotide primer comprising the same nucleotide sequence as the 5′ primer-specific portion of the ligated product sequence and (b) a second secondary oligonucleotide primer comprising a nucleotide sequence that is complementary to the 3′ primer-specific portion of the ligated product sequence. The ligated product sequences and the one or more secondary oligonucleotide primer sets are blended with one or more enzymes capable of digesting deoxyuracil (dU)-containing nucleic acid molecules, a deoxynucleotide mix including dUTP, and a DNA polymerase to form one or more second polymerase chain reaction mixtures. The one or more second polymerase chain reaction mixtures are subjected to conditions suitable for digesting deoxyuracil (dU)-containing nucleic acid molecules present in the second polymerase chain reaction mixtures and for carrying out one or more polymerase chain reaction cycles comprising a denaturation treatment, a hybridization treatment, and an extension treatment thereby forming secondary polymerase chain reaction products. The method further comprises detecting and distinguishing the secondary polymerase chain reaction products in the one or more reactions thereby identifying one or more target miRNA molecules differing in sequence from other miRNA molecules in the sample by one or more bases.

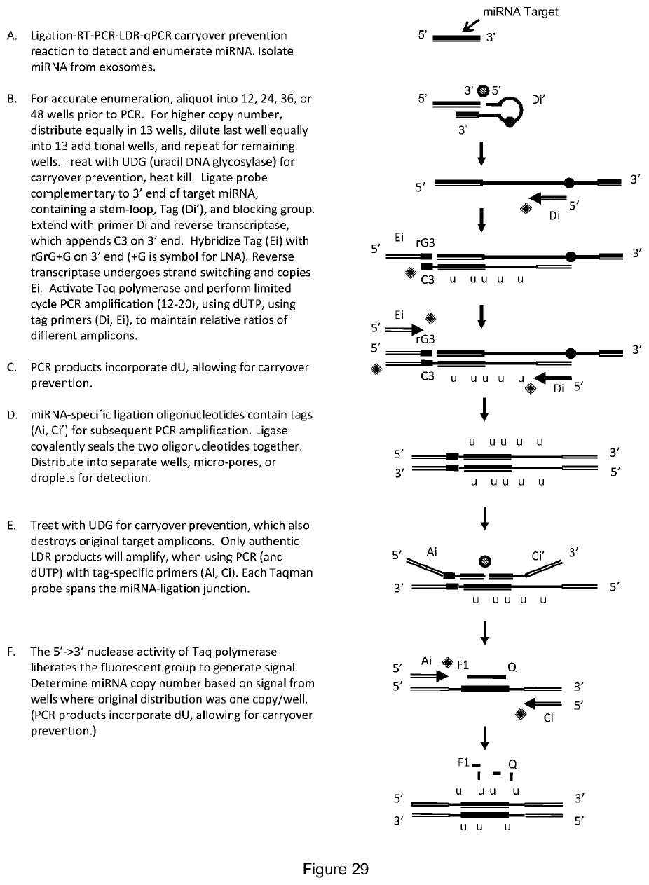

FIG. 29 illustrates an embodiment of this aspect of the present application.

FIG. 29 illustrates an exemplary Ligation-RT-PCR-LDR-qPCR carryover prevention reaction to quantify miRNA. For accurate enumeration, aliquot into 12, 24, 36, or 48 wells prior to PCR. For higher copy number, distribute equally in 13 wells, dilute last well equally into 13 additional wells, and repeat for remaining wells. This method involves isolating miRNA from exosomes and treating with UDG for carryover prevention (FIG. 29, step B). An oligonucleotide probe having a portion that is complementary to the 3′ end of the target miRNA, and containing a stem-loop, tag (Di′), and blocking group (filled circle) is ligated at its 5′ end to the 3′ end of the target miRNA. The ligation product comprises the miRNA, Di′ tag, the blocking group, and a sequence complementary to the 3′ portion of the miRNA (FIG. 29, step B). A reverse transcriptase such as Moloney Murine Leukemia Virus Reverse Transcriptase (M-MLV RT, New England Biolabs), or Superscript II or III Reverse Transcriptase (Life Technologies) extends primer (Di) to make a full-length copy of the target, and appends three C bases to the 3′ end of extended target sequence (FIG. 29, step B). Tag oligonucleotide (Ei) having the 3′ rGrG+G (+G is the symbol for LNA) hybridizes to the three C bases of the extended target sequence as shown in FIG. 29, step B. The reverse transcriptase undergoes strand switching and copies the Ei tag sequence. Activate Taq polymerase and perform limited cycle PCR amplification (12-20), using dUTP, using tag primers (Di, Ei), to maintain relative ratios of different amplicons. The PCR products incorporate dU, allowing for carryover prevention (FIG. 29, step C).

As shown in FIG. 29, step D miRNA sequence-specific ligation probes containing primer-specific portions (Ai, Ci′) suitable for subsequent PCR amplification, hybridize to their corresponding target sequence in a base-specific manner. Following ligation, the ligation products can be detected using pairs of matched primers Ai and Ci, and TaqMan™ probes that span the ligation junction as described supra for FIG. 2 (see FIG. 29, steps D-F), or using other suitable means known in the art.

Another aspect of the present application is directed to a method for identifying, in a sample, one or more target micro-ribonucleic acid (miRNA) molecules differing in sequence from other miRNA molecules in the sample by one or more bases. The method involves providing a sample containing one or more target miRNA molecules potentially differing in sequence from other miRNA molecules in the sample by one or more bases and providing one or more enzymes capable of digesting deoxyuracil (dU)-containing nucleic acid molecules present in the sample. The sample is contacted with one or more enzymes capable of digesting dU-containing nucleic acid molecules potentially present in the sample, and the contacted sample is blended with a ligase and one or more first oligonucleotide probes comprising a 5′ phosphate, a 5′ stem-loop portion, an internal primer-specific portion within the loop region, a blocking group, and a 3′ nucleotide sequence that is complementary to a 3′ portion of the target miRNA molecule sequence to form one or more ligation reaction mixtures. The method further involves ligating, in the one or more ligation reaction mixtures, the one or more target miRNA molecules at their 3′ end to the 5′ phosphate of the one or more first oligonucleotide probes to generate chimeric nucleic acid molecules comprising the target miRNA molecule sequence, if present in the sample, appended to the one or more first oligonucleotide probes. One or more primary oligonucleotide primer sets are then provided. Each primer set comprises (a) a first primary oligonucleotide primer comprising a nucleotide sequence that is complementary to the internal primer-specific portion of the first oligonucleotide probe, and (b) a second primary oligonucleotide primer comprising a 5′ primer-specific portion and a 3′ portion, wherein the second primary oligonucleotide primer may be the same or may differ from other second primary oligonucleotide primers in other sets. The one or more ligation reaction mixtures comprising chimeric nucleic acid molecules, the one or more primary oligonucleotide primer sets, a deoxynucleotide mix, and a reverse transcriptase and a DNA polymerase or a DNA polymerase with reverse-transcriptase activity are blended to form one or more reverse-transcription/polymerase chain reaction mixtures. The one or more reverse-transcription/polymerase chain reaction mixtures are subjected to conditions suitable for generating complementary deoxyribonucleic acid (cDNA) molecules to the chimeric nucleic acid molecules, and to one or more polymerase chain reaction cycles comprising a denaturation treatment, a hybridization treatment, and an extension treatment thereby forming one or more different primary reverse-transcription/polymerase chain reaction products comprising the 5′ primer-specific portion, a nucleotide sequence corresponding to the target miRNA molecule sequence, and the complement of the internal primer-specific portion, and complements thereof. The method further comprises providing one or more secondary oligonucleotide primer sets. Each secondary oligonucleotide primer set comprises (a) a first secondary oligonucleotide primer having a 5′ primer-specific portion and a 3′ portion that is complementary to a portion of an extension product formed from the first primary oligonucleotide primer and (b) a second secondary oligonucleotide primer having a 5′ primer-specific portion and a 3′ portion that comprises a nucleotide sequence that is complementary to a portion of an extension product formed from the first secondary oligonucleotide primer. The primary reverse-transcription/polymerase chain reaction products, the one or more secondary oligonucleotide primer sets, a deoxynucleotide mix, and a DNA polymerase are blended to form one or more first polymerase chain reaction mixtures, and the one or more first polymerase chain reaction mixtures are subjected to conditions suitable for two or more polymerase chain reaction cycles comprising a denaturation treatment, a hybridization treatment, and an extension treatment, thereby forming first polymerase chain reaction products comprising a 5′ primer-specific portion of the first secondary oligonucleotide primer, a nucleotide sequence corresponding to the target miRNA molecule sequence or a complement thereof, and a complement of the other 5′ primer-specific portion second secondary oligonucleotide primer. The method further involves providing one or more tertiary oligonucleotide primer sets. Each tertiary oligonucleotide primer set comprises (a) a first tertiary oligonucleotide primer comprising the same nucleotide sequence as the 5′ primer-specific portion of the first polymerase chain reaction products or their complements and (b) a second tertiary oligonucleotide primer comprising a nucleotide sequence that is complementary to the 3′ primer-specific portion of the first polymerase chain reaction products or their complements. The first polymerase chain reaction process products, the one or more tertiary oligonucleotide primer sets, the one or more enzymes capable of digesting deoxyuracil (dU)-containing nucleic acid molecules, a deoxynucleotide mix including dUTP, and a DNA polymerase are blended to form one or more second polymerase chain reaction mixtures, and the one or more second polymerase chain reaction mixtures are subjected to conditions suitable for digesting deoxyuracil (dU) containing nucleic acid molecules present in the second polymerase chain reaction mixtures and for carrying out one or more polymerase chain reaction cycles comprising a denaturation treatment, a hybridization treatment, and an extension treatment thereby forming second polymerase chain reaction products. The method further comprises detecting and distinguishing the second polymerase chain reaction products, thereby identifying one or more target miRNA molecules differing in sequence from other miRNA molecules in the sample by one or more bases.

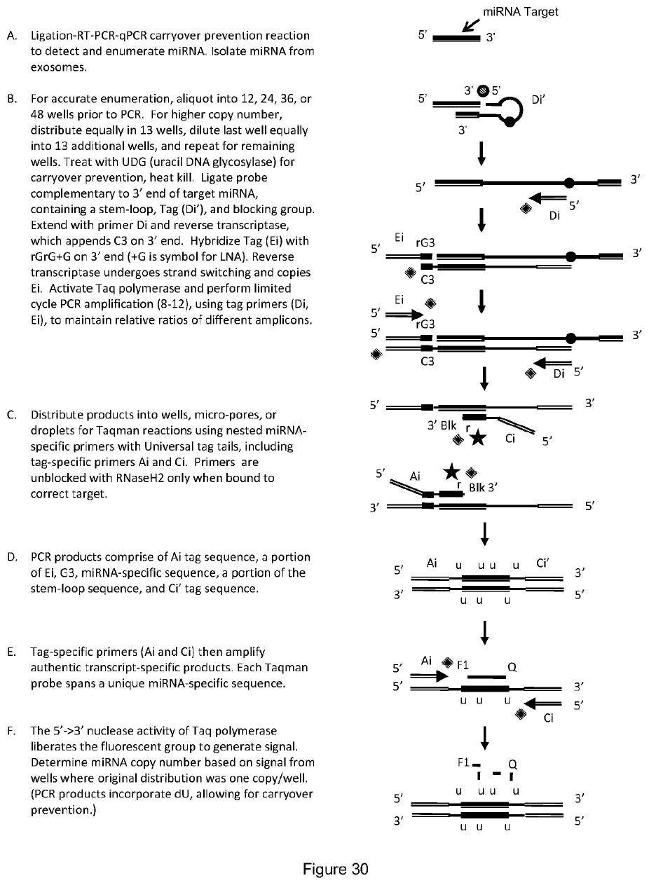

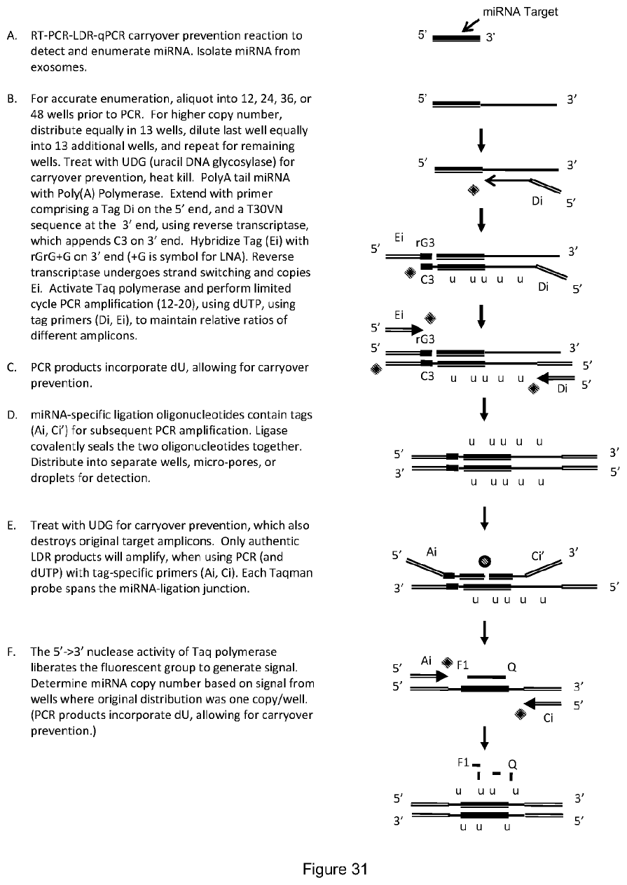

FIG. 30 illustrates an embodiment of this aspect of the present application.

FIG. 30 illustrates an exemplary Ligation-RT-PCR-qPCR carryover prevention reaction to quantify miRNA. For accurate enumeration, aliquot into 12, 24, 36, or 48 wells prior to PCR. For higher copy number, distribute equally in 13 wells, dilute last well equally into 13 additional wells, and repeat for remaining wells. This method involves isolating miRNA from exosomes and treating with UDG for carryover prevention (FIG. 30, step B). An oligonucleotide probe having a portion that is complementary to the 3′ end of the target miRNA, and containing a stem-loop, tag (Di′), and blocking group (filled circle) is ligated at its 5′ end to the 3′ end of the target miRNA. The ligation product comprises the miRNA, Di′ tag, the blocking group, and a sequence complementary to the 3′ portion of the miRNA (FIG. 30, step B). A reverse transcriptase extends primer (Di) to make a full-length copy of the target and appends three C bases to the 3′ end of extended target sequence (FIG. 30, step B). Tag oligonucleotide (Ei) having the 3′ rGrG+G hybridizes to the three C bases of the extended target sequence as shown in FIG. 30, step B. The reverse transcriptase undergoes strand switching and copies the Ei tag sequence. Activate Taq polymerase and perform limited cycle PCR amplification (8-20), using tag primers (Di, Ei), to maintain relative ratios of different amplicons.

As shown in FIG. 30, step C, following the limited cycle PCR, the PCR products are aliquoted into separate wells, micro-pores or droplets containing Taqman™ probes across the miRNA target region, miRNA-specific (forward) primers comprising 5′ primer-specific portions (Ai), cDNA-specific (reverse) primers comprising 5′ primer-specific portions (Ci) and matching primers Ai and Ci. These primers combine to amplify the target miRNA sequence, if present in the sample (FIG. 30, step C). Primers are unblocked with RNaseH2 only when bound to correct target. Following PCR, the products can be detected using pairs of matched primers Ai and Ci, and TaqMan™ probes that span the target miRNA regions as described supra for FIG. 4 (see FIG. 30, steps C-F), or using other suitable means known in the art.

In one embodiment, the 3′ portion of the second primary oligonucleotide primer comprises ribo-G and/or G nucleotide analogue, wherein the reverse transcriptase appends two or three cytosine nucleotides to the 3′ end of the complementary deoxyribonucleic acid products of the target miRNAs, enabling transient hybridization to the 3′ end of the second primary oligonucleotide primer, enabling the reverse transcriptase to undergo strand switching and to extend the complementary deoxyribonucleic acid products to include the complementary sequence of the 5′ primer-specific portion of the second primary oligonucleotide primer to form the one or more different first polymerase chain reaction products comprising a 5′ primer-specific portion, a nucleotide sequence portion corresponding to the target miRNA molecule sequence or a complement thereof, a further portion, and a complement of the other 5′ primer-specific portion.

In another embodiment, the 3′ portion of the second primary oligonucleotide primers contains from 6 to 14 bases comprising, from 5′ to 3′, three ribo-G or G bases, followed by additional bases that are the same as the 5′ end of the target miRNA sequences, wherein the reverse transcriptase appends two or three cytosine residues to the 3′ end of the initial complementary deoxyribonucleic acid extension products of the target miRNAs, and wherein subsequent to when the denaturation treatment of the polymerase chain reaction is initiated the conditions are adjusted to enable transient hybridization to the 3′ end of the second primary oligonucleotide primers to the 3′ end of the complementary deoxyribonucleic acid extension products, allowing for extension of either one or both the second primary oligonucleotide primers and the complementary deoxyribonucleic acid extension products to form the one or more different primary reverse-transcription/polymerase chain reaction products comprising a 5′ primer-specific portion, a nucleotide sequence portion corresponding to the target miRNA molecule sequence or a complement thereof, a further portion, and a complement of the other 5′ primer-specific portion.

In certain embodiments, the second oligonucleotide probe of the oligonucleotide probe set further comprises a unitaq detection portion, thereby forming ligated product sequences comprising the 5′ primer-specific portion, the target-specific portions, the unitaq detection portion, and the 3′ primer-specific portion. In accordance with this embodiment, one or more unitaq detection probes are provided, wherein each unitaq detection probe hybridizes to a complementary unitaq detection portion and said detection probe comprises a quencher molecule and a detectable label separated from the quencher molecule. The one or more unitaq detection probes are added to the second polymerase chain reaction mixture, and the one or more unitaq detection probes are hybridized to complementary unitaq detection portions on the ligated product sequence or complement thereof during said subjecting the second polymerase chain reaction mixture to conditions suitable for one or more polymerase chain reaction cycles, wherein the quencher molecule and the detectable label are cleaved from the one or more unitaq detection probes during the extension treatment and said detecting involves the detection of the cleaved detectable label.

In another embodiment, one primary oligonucleotide primer or one secondary oligonucleotide primer further comprises a unitaq detection portion, thereby forming extension product sequences comprising the 5′ primer-specific portion, the target-specific portions, the unitaq detection portion, and the complement of the other 5′ primer-specific portion, and complements thereof. In accordance with this embodiment, one or more unitaq detection probes are provided, wherein each unitaq detection probe hybridizes to a complementary unitaq detection portion and said detection probe comprises a quencher molecule and a detectable label separated from the quencher molecule. The one or more unitaq detection probes are added to the one or more first or second polymerase chain reaction mixtures, and the one or more unitaq detection probes are hybridized to complementary unitaq detection portions on the ligated product sequence or complement thereof during polymerase chain reaction cycles after the first polymerization chain reaction, wherein the quencher molecule and the detectable label are cleaved from the one or more unitaq detection probes during the extension treatment and said detecting involves the detection of the cleaved detectable label.

In another embodiment, one or both oligonucleotide probes of the oligonucleotide probe set comprises a portion that has no or one nucleotide sequence mismatch when hybridized in a base-specific manner to the target nucleic acid sequence or bisulfite-converted methylated nucleic acid sequence or complement sequence thereof, but have one or more additional nucleotide sequence mismatches that interferes with ligation when said oligonucleotide probe hybridizes in a base-specific manner to a corresponding nucleotide sequence portion in the wildtype nucleic acid sequence or bisulfite-converted unmethylated nucleic acid sequence or complement sequence thereof.

In one embodiment, the 3′ portion of the first oligonucleotide probe of the oligonucleotide probe set comprises a cleavable nucleotide or nucleotide analogue and a blocking group, such that the 3′ end is unsuitable for polymerase extension or ligation. The cleavable nucleotide or nucleotide analog of the first oligonucleotide probe is cleaved when said probe is hybridized to it complementary target nucleotide sequence of the primary extension product, thereby liberating a 3′OH on the first oligonucleotide probe prior to the ligating.

The one or more first oligonucleotide probe of the oligonucleotide probe set may comprises a sequence that differs from the target nucleic acid sequence or bisulfite-converted methylated nucleic acid sequence or complement sequence thereof, said difference is located two or three nucleotide bases from the liberated free 3′OH end.

In a further embodiment, the second oligonucleotide probe has, at its 5′ end, an overlapping identical nucleotide with the 3′ end of the first oligonucleotide probe, and, upon hybridization of the first and second oligonucleotide probes of a probe set at adjacent positions on a complementary target nucleotide sequence of a primary extension product to form a junction, the overlapping identical nucleotide of the second oligonucleotide probe forms a flap at the junction with the first oligonucleotide probe. This embodiment further comprises cleaving the overlapping identical nucleotide of the second oligonucleotide probe with an enzyme having 5′ nuclease activity thereby liberating a phosphate at the 5′ end of the second oligonucleotide probe prior to said ligating.

In other embodiments, the one or more oligonucleotide probe sets further comprise a third oligonucleotide probe having a target-specific portion, wherein the second and third oligonucleotide probes of a probe set are configured to hybridize adjacent to one another on the target nucleotide sequence with a junction between them to allow ligation between the second and third oligonucleotide probes to form a ligated product sequence comprising the first, second, and third oligonucleotide probes of a probe set.

In certain embodiments, the sample is selected from the group consisting of tissue, cells, serum, blood, plasma, amniotic fluid, sputum, urine, bodily fluids, bodily secretions, bodily excretions, cell-free circulating nucleic acids, cell-free circulating tumor nucleic acids, cell-free circulating fetal nucleic acids in pregnant woman, circulating tumor cells, tumor, tumor biopsy, and exosomes.

The one or more target nucleotide sequences may be low-abundance nucleic acid molecules comprising one or more nucleotide base mutations, insertions, deletions, translocations, splice variants, miRNA variants, alternative transcripts, alternative start sites, alternative coding sequences, alternative non-coding sequences, alternative splicings, exon insertions, exon deletions, intron insertions, or other rearrangement at the genome level and/or methylated nucleotide bases.

As used herein “low abundance nucleic acid molecule” refers to a target nucleic acid molecule that is present at levels as low as 1% to 0.01% of the sample. In other words, a low abundance nucleic acid molecule with one or more nucleotide base mutations, insertions, deletions, translocations, splice variants, miRNA variants, alternative transcripts, alternative start sites, alternative coding sequences, alternative non-coding sequences, alternative splicings, exon insertions, exon deletions, intron insertions, other rearrangement at the genome level, and/or methylated nucleotide bases can be distinguished from a 100 to 10,000-fold excess of nucleic acid molecules in the sample (i.e., high abundance nucleic acid molecules) having a similar nucleotide sequence as the low abundance nucleic acid molecules but without the one or more nucleotide base mutations, insertions, deletions, translocations, splice variants, miRNA variants, alternative transcripts, alternative start sites, alternative coding sequences, alternative non-coding sequences, alternative splicings, exon insertions, exon deletions, intron insertions, other rearrangement at the genome level, and/or methylated nucleotide bases.

In some embodiments of the present invention, the copy number of one or more low abundance target nucleotide sequences are quantified relative to the copy number of high abundance nucleic acid molecules in the sample having a similar nucleotide sequence as the low abundance nucleic acid molecules. In other embodiments of the present invention, the one or more target nucleotide sequences are quantified relative to other nucleotide sequences in the sample. In other embodiments of the present invention, the relative copy number of one or more target nucleotide sequences is quantified. Methods of relative and absolute (i.e., copy number) quantitation are well known in the art.

The low abundance target nucleic acid molecules to be detected can be present in any biological sample, including, without limitation, tissue, cells, serum, blood, plasma, amniotic fluid, sputum, urine, bodily fluids, bodily secretions, bodily excretions, cell-free circulating nucleic acids, cell-free circulating tumor nucleic acids, cell-free circulating fetal nucleic acids in pregnant woman, circulating tumor cells, tumor, tumor biopsy, and exosomes.

The methods of the present invention are suitable for diagnosing or prognosing a disease state and/or distinguishing a genotype or disease predisposition.

Another aspect of the present application is directed to a method for identifying, in a sample, one or more target micro-ribonucleic acid (miRNA) molecules differing in sequence from other miRNA molecules in the sample by one or more bases. The method involves providing a sample containing one or more target miRNA molecules potentially differing in sequence from other miRNA molecules in the sample by one or more bases and providing one or more enzymes capable of digesting deoxyuracil (dU)-containing nucleic acid molecules present in the sample. The sample is contacted with one or more enzymes capable of digesting dU-containing nucleic acid molecules potentially present in the sample. The contacted sample is blended with ATP and a Poly(A) polymerase to form a Poly(A) polymerase reaction mixture, and the Poly(A) polymerase reaction mixture is subjected to conditions suitable for appending homopolymer A to the 3′ ends of the one or more target miRNA molecules potentially present in the sample. The method further involves providing one or more primary oligonucleotide primer sets. Each primer set comprises (a) a first primary oligonucleotide primer comprising a 5′ primer-specific portion, an internal poly dT portion, and a 3′ portion comprising from 1 to 10 bases complementary to the 3′ end of the target miRNA, wherein the first primary oligonucleotide primer may be the same or may differ from other first primary oligonucleotide primers in other sets, and (b) a second primary oligonucleotide primer comprising a 5′ primer-specific portion and a 3′ portion, wherein the second primary oligonucleotide primer may be the same or may differ from other second primary oligonucleotide primers in other sets. The Poly(A) polymerase reaction mixture, the one or more primary oligonucleotide primer sets, the one or more enzymes capable of digesting deoxyuracil (dU)-containing nucleic acid molecules in the sample, a deoxynucleotide mix including dUTP, and a reverse transcriptase and a DNA polymerase or a DNA polymerase with reverse-transcriptase activity are blended to form one or more reverse-transcription/polymerase chain reaction mixtures. The one or more reverse-transcription/polymerase chain reaction mixtures are subjected to conditions suitable for digesting deoxyuracil (dU)-containing nucleic acid molecules present in the reverse-transcription/polymerase chain reaction mixtures, then to conditions suitable for generating complementary deoxyribonucleic acid (cDNA) molecules to the target miRNA sequences with 3′ polyA tails, and to one or more polymerase chain reaction cycles comprising a denaturation treatment, a hybridization treatment, and an extension treatment thereby forming one or more different reverse-transcription/polymerase chain reaction products comprising the 5′ primer-specific portion of the second primary oligonucleotide primer, a nucleotide sequence corresponding to the target miRNA molecule sequence, a poly dA region, and the complement of the 5′ primer-specific portion of the first primary oligonucleotide primer, and complements thereof. The method further comprises providing one or more oligonucleotide probe sets. Each probe set comprises (a) a first oligonucleotide probe having a 5′ primer-specific portion and a 3′ target sequence-specific portion, and (b) a second oligonucleotide probe having a 5′ target sequence-specific portion, a portion complementary to the one or more reverse-transcription/polymerase chain reaction products, and a 3′ primer-specific portion, wherein the first and second oligonucleotide probes of a probe set are configured to hybridize, in a base specific manner, to complementary portions of the one or more reverse-transcription/polymerase chain reaction products corresponding to the target miRNA molecule sequence, or complement thereof. The one or more reverse-transcription/polymerase chain reaction products are contacted with a ligase and the one or more oligonucleotide probe sets to form one or more ligation reaction mixtures, and the one or more ligation reaction mixtures are subjected to one or more ligation reaction cycles whereby the first and second oligonucleotide probes of the one or more oligonucleotide probe sets, when hybridized to their complement, are ligated together to form ligated product sequences in the ligation reaction mixture, wherein each ligated product sequence comprises the 5′ primer-specific portion, the target-specific portions, and the 3′ primer-specific portion. The method further involves providing one or more secondary oligonucleotide primer sets. Each secondary oligonucleotide primer set comprises (a) a first secondary oligonucleotide primer comprising the same nucleotide sequence as the 5′ primer-specific portion of the ligated product sequence and (b) a second secondary oligonucleotide primer comprising a nucleotide sequence that is complementary to the 3′ primer-specific portion of the ligated product sequence. The ligated product sequences and the one or more secondary oligonucleotide primer sets are blended with one or more enzymes capable of digesting deoxyuracil (dU)-containing nucleic acid molecules, a deoxynucleotide mix including dUTP, and a DNA polymerase to form one or more first polymerase chain reaction mixtures, and the one or more first polymerase chain reaction mixtures are subjected to conditions suitable for digesting deoxyuracil (dU)-containing nucleic acid molecules present in the first polymerase chain reaction mixtures and for carrying out one or more polymerase chain reaction cycles comprising a denaturation treatment, a hybridization treatment, and an extension treatment thereby forming secondary polymerase chain reaction products. The method further comprises detecting and distinguishing the secondary polymerase chain reaction products, thereby identifying one or more target miRNA molecules differing in sequence from other miRNA molecules in the sample by one or more bases.

FIG. 31 illustrates an embodiment of this aspect of the present application.

FIG. 31 illustrates an exemplary RT-PCR-LDR-qPCR carryover prevention reaction to quantify miRNA. For accurate enumeration, aliquot into 12, 24, 36, or 48 wells prior to PCR. For higher copy number, distribute equally in 13 wells, dilute last well equally into 13 additional wells, and repeat for remaining wells. This method involves isolating miRNA from exosomes and treating with UDG for carryover prevention (FIG. 31, step B). PolyA tail miRNA with E. coli Poly(A) polymerase. A reverse transcriptase extends primer comprising a Tag Di on the 5′ end, and a T30VN sequence at the 3′ end, to make a full-length copy of the target, and appends three C bases to the 3′ end of extended target sequence (FIG. 31, step B). Tag oligonucleotide (Ei) having the 3′ rGrG+G hybridizes to the three C bases of the extended target sequence as shown in FIG. 31, step B. The reverse transcriptase undergoes strand switching and copies the Ei tag sequence. Activate Taq polymerase and perform limited cycle PCR amplification (12-20), using dUTP, using tag primers (Di, Ei), to maintain relative ratios of different amplicons. The PCR products incorporate dU, allowing for carryover prevention (FIG. 31, step C).

As shown in FIG. 31, step D miRNA sequence-specific ligation probes containing primer-specific portions (Ai, Ci′) suitable for subsequent PCR amplification, hybridize to their corresponding target sequence in a base-specific manner. Following ligation, the ligation products can be detected using pairs of matched primers Ai and Ci, and TaqMan™ probes that span the ligation junction as described supra for FIG. 2 (see FIG. 31, steps D-F), or using other suitable means known in the art.

Another aspect of the present application is directed to a method for identifying, in a sample, one or more target micro-ribonucleic acid (miRNA) molecules differing in sequence from other miRNA molecules in the sample by one or more bases. The method involves providing a sample containing one or more target miRNA molecules potentially differing in sequence from other miRNA molecules in the sample by one or more bases and providing one or more enzymes capable of digesting deoxyuracil (dU)-containing nucleic acid molecules present in the sample. The sample is contacted with one or more enzymes capable of digesting dU-containing nucleic acid molecules potentially present in the sample. The contacted sample is blended with ATP and a Poly(A) polymerase to form a Poly(A) polymerase reaction mixture, and the Poly(A) polymerase reaction mixture is subjected to conditions suitable for appending a homopolymer A to the 3′ ends of the one or more target miRNA molecules potentially present in the sample. The method further involves providing one or more primary oligonucleotide primer sets. Each primer set comprises (a) a first primary oligonucleotide primer comprising a 5′ primer-specific portion, an internal poly dT portion, and a 3′ portion comprising from 1 to 10 bases complementary to the 3′ end of the target miRNA, wherein the first primary oligonucleotide primer may be the same or may differ from other first primary oligonucleotide primers in other sets, and (b) a second primary oligonucleotide primer comprising a 5′ primer-specific portion and a 3′ portion, wherein the second primary oligonucleotide primer may be the same or may differ from other second primary oligonucleotide primers in other sets. The Poly(A) polymerase reaction mixture potentially comprising target miRNA sequences is blended with 3′ polyA tails, the one or more primary oligonucleotide primer sets, a deoxynucleotide mix, and a reverse transcriptase and a DNA polymerase or a DNA polymerase with reverse-transcriptase activity to form one or more reverse-transcription/polymerase chain reaction mixtures. The one or more reverse-transcription/polymerase chain reaction mixtures are subjected to conditions suitable for generating complementary deoxyribonucleic acid (cDNA) molecules to the target miRNA sequences with 3′ polyA tails, and to one or more polymerase chain reaction cycles comprising a denaturation treatment, a hybridization treatment, and an extension treatment, thereby forming one or more different reverse-transcription/polymerase chain reaction products comprising the 5′ primer-specific portion of the second primary oligonucleotide primer, a nucleotide sequence corresponding to the target miRNA molecule sequence, a poly dA region, and the complement of the 5′ primer-specific portion of the first primary oligonucleotide primer, and complements thereof. The method further comprises providing one or more secondary oligonucleotide primer sets. Each secondary oligonucleotide primer set comprises (a) a first secondary oligonucleotide primer having a 5′ primer-specific portion and a 3′ portion that is complementary to a portion of a reverse-transcription/polymerase chain reaction product formed from the first primary oligonucleotide primer and (b) a second secondary oligonucleotide primer having a 5′ primer-specific portion and a 3′ portion that comprises a nucleotide sequence that is complementary to a portion of a reverse-transcription/polymerase chain reaction product formed from the first secondary oligonucleotide primer. The reverse-transcription/polymerase chain reaction products, the one or more secondary oligonucleotide primer sets, a deoxynucleotide mix, and a DNA polymerase are blended to form one or more first polymerase chain reaction mixtures, and the one or more first polymerase chain reaction mixtures are subjected to conditions suitable for two or more polymerase chain reaction cycles comprising a denaturation treatment, a hybridization treatment, and an extension treatment, thereby forming first polymerase chain reaction products comprising a 5′ primer-specific portion, a nucleotide sequence corresponding to the target miRNA molecule sequence or a complement thereof, and a complement of the other 5′ primer-specific portion. The method further involves providing one or more tertiary oligonucleotide primer sets. Each tertiary oligonucleotide primer set comprises (a) a first tertiary oligonucleotide primer comprising the same nucleotide sequence as the 5′ primer-specific portion of the first polymerase chain reaction product sequence and (b) a second tertiary oligonucleotide primer comprising a nucleotide sequence that is complementary to the 3′ primer-specific portion of the first polymerase chain reaction product sequence. The first polymerase chain reaction products, the one or more tertiary oligonucleotide primer sets, the one or more enzymes capable of digesting deoxyuracil (dU) containing nucleic acid molecules, a deoxynucleotide mix including dUTP, and a DNA polymerase are blended to form one or more second polymerase chain reaction mixtures. The one or more second polymerase chain reaction mixtures are subjected to conditions suitable for digesting deoxyuracil (dU)-containing nucleic acid molecules present in the first polymerase chain reaction mixtures, and one or more polymerase chain reaction cycles comprising a denaturation treatment, a hybridization treatment, and an extension treatment thereby forming second polymerase chain reaction products. The method further comprises detecting and distinguishing the second polymerase chain reaction products in the one or more reactions thereby identifying one or more target miRNA molecules differing in sequence from other miRNA molecules in the sample by one or more bases.

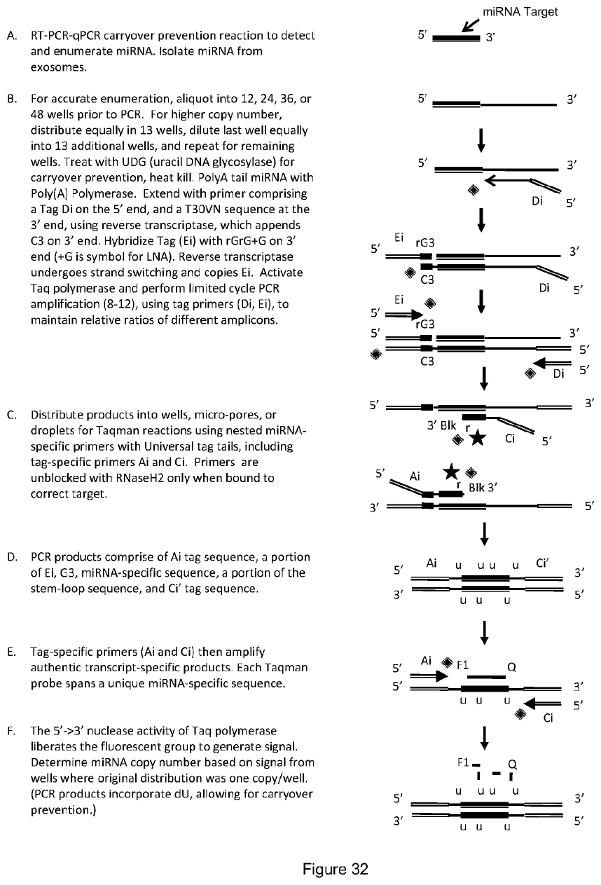

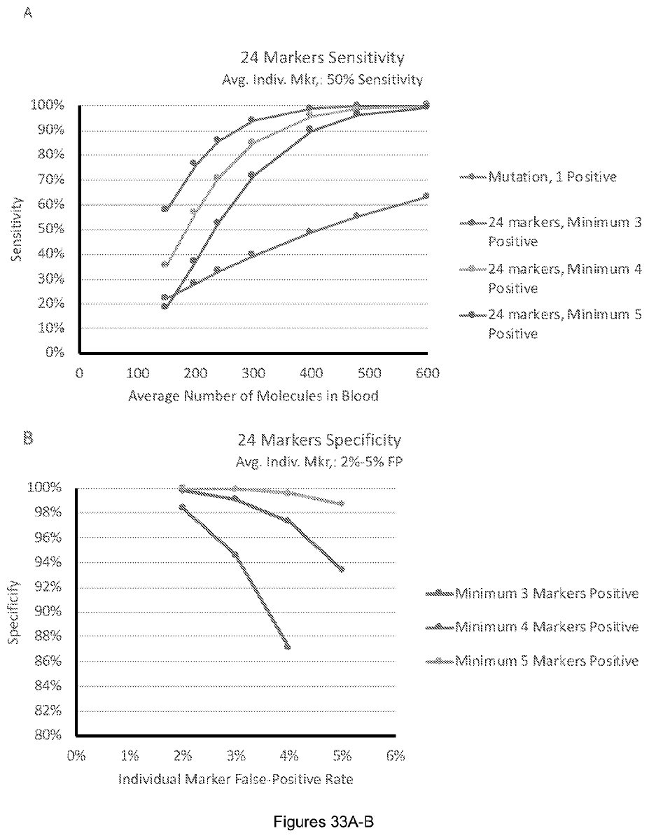

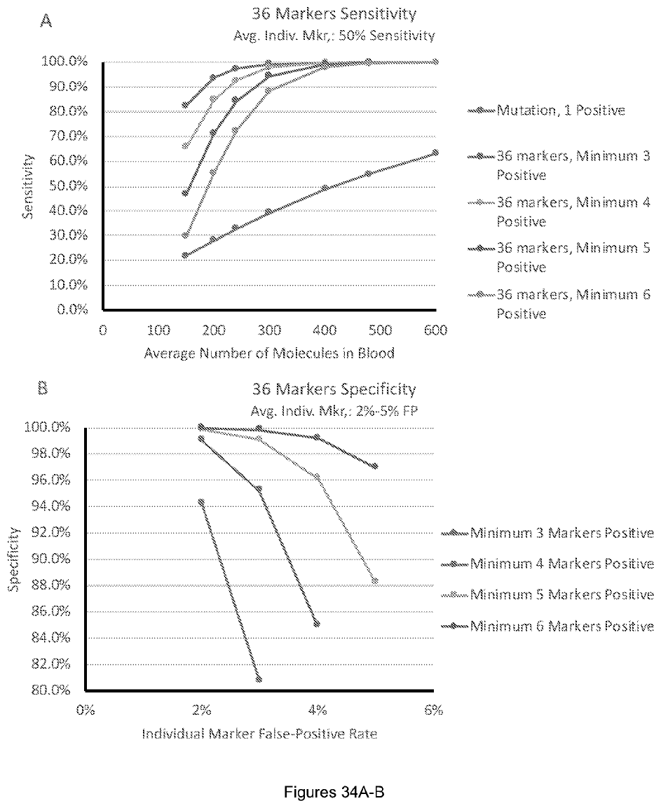

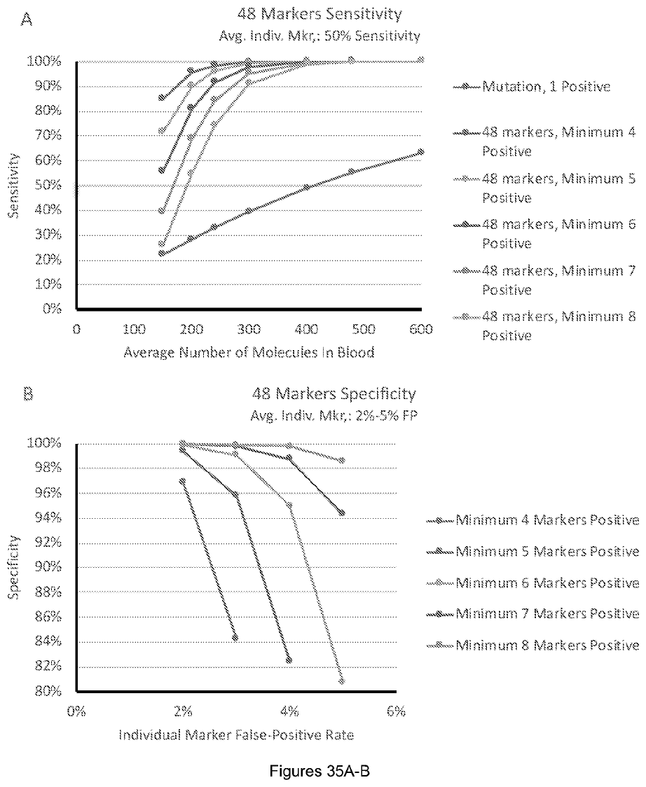

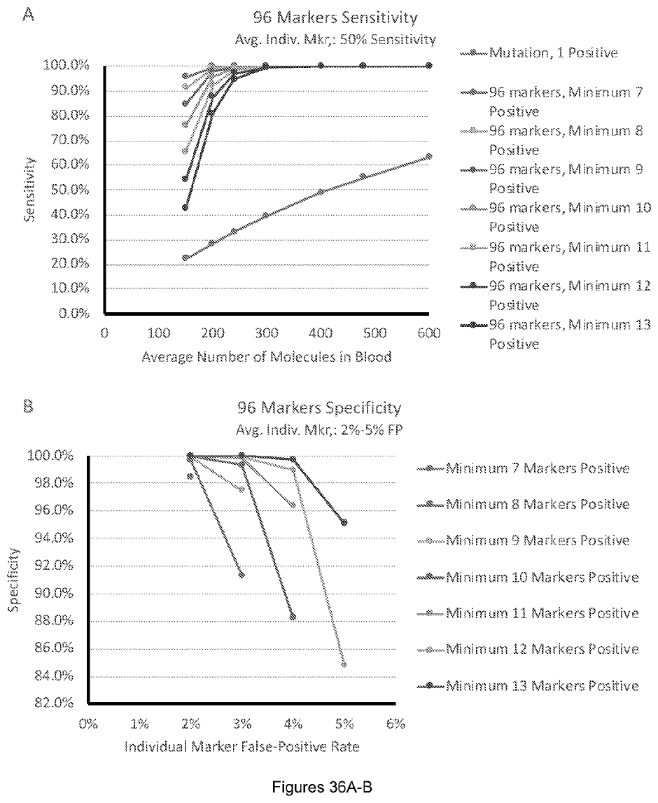

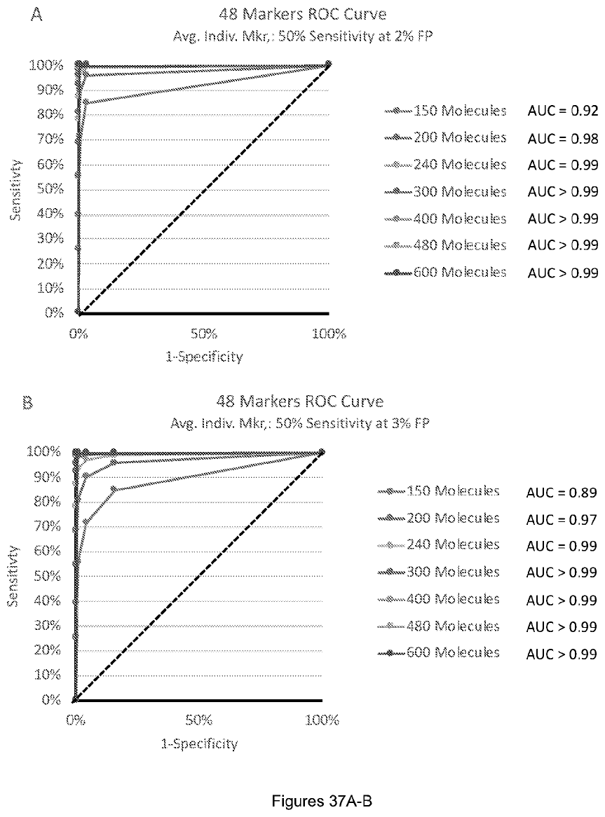

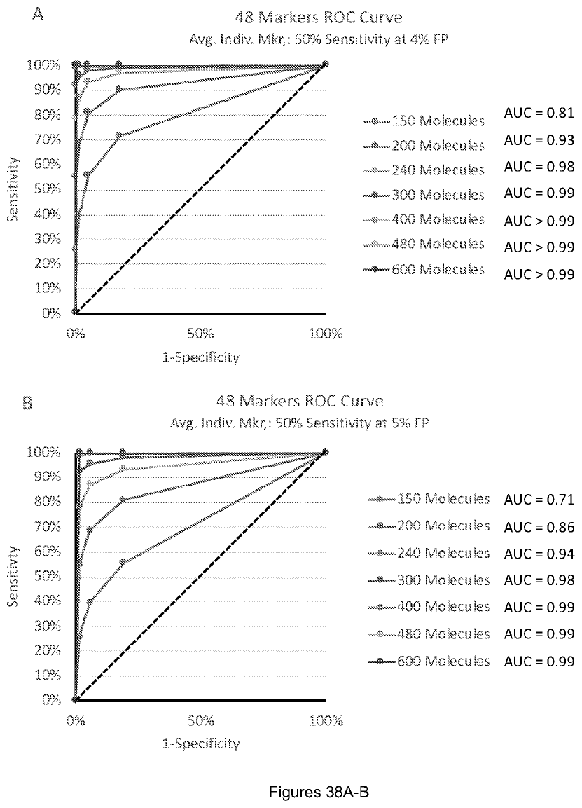

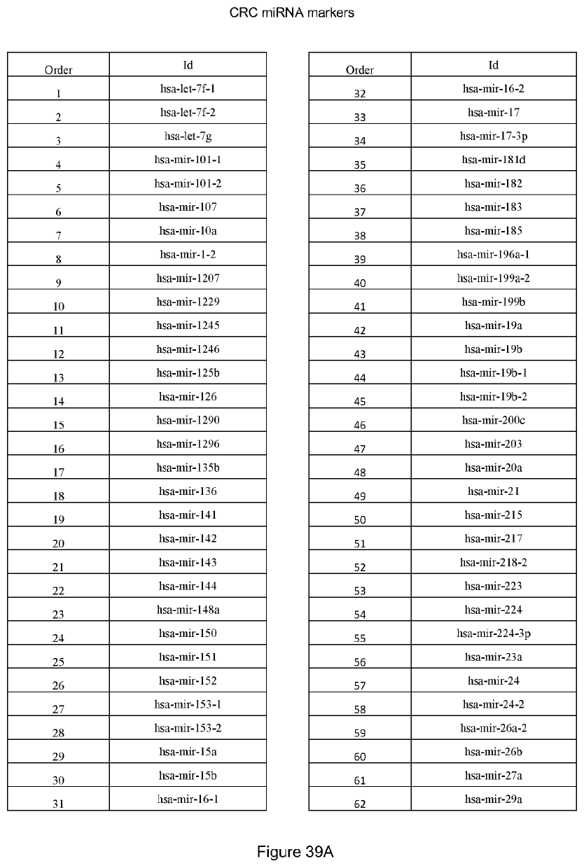

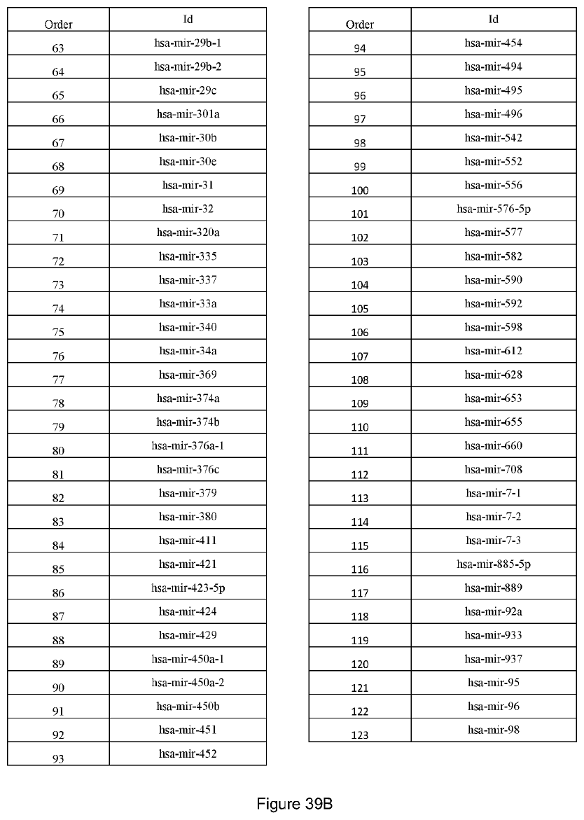

FIG. 32 illustrates an embodiment of this aspect of the present application.

FIG. 32 illustrates an exemplary RT-PCR-qPCR carryover prevention reaction to quantify miRNA. For accurate enumeration, aliquot into 12, 24, 36, or 48 wells prior to PCR. For higher copy number, distribute equally in 13 wells, dilute last well equally into 13 additional wells, and repeat for remaining wells. This method involves isolating miRNA from exosomes and treating with UDG for carryover prevention (FIG. 32, step B). PolyA tail miRNA with E. coli Poly(A) polymerase. A reverse transcriptase extends primer comprising a Tag Di on the 5′ end, and a T30VN sequence at the 3′ end, to make a full-length copy of the target, and appends three C bases to the 3′ end of extended target sequence (FIG. 32, step B). Tag oligonucleotide (Ei) having the 3′ rGrG+G hybridizes to the three C bases of the extended target sequence as shown in FIG. 32, step B. The reverse transcriptase undergoes strand switching and copies the Ei tag sequence. Activate Taq polymerase and perform limited cycle PCR amplification (8-20), using dUTP, using tag primers (Di, Ei), to maintain relative ratios of different amplicons.

As shown in FIG. 32, step C, following the limited cycle PCR, the PCR products are aliquoted into separate wells, micro-pores or droplets containing Taqman™ probes across the miRNA target region, miRNA-specific (forward) primers comprising 5′ primer-specific portions (Ai), cDNA-specific (reverse) primers comprising 5′ primer-specific portions (Ci) and matching primers Ai and Ci. These primers combine to amplify the target miRNA sequence, if present in the sample (FIG. 32, step C). Primers are unblocked with RNaseH2 only when bound to correct target. Following PCR, the products can be detected using pairs of matched primers Ai and Ci, and TaqMan™ probes that span the target miRNA regions as described supra for FIG. 6 (see FIG. 32, steps C-F), or using other suitable means known in the art.

Another aspect of the present application is directed to a method of diagnosing or prognosing a disease state of cells or tissue based on identifying the presence or level of a plurality of disease-specific and/or cell/tissue-specific DNA, RNA, and/or protein markers in a biological sample of an individual. The plurality of markers is in a set comprising from 6-12 markers, 12-24 markers, 24-36 markers, 36-48 markers, 48-72 markers, 72-96 markers, or >96 markers. Each marker in a given set is selected by having any one or more of the following criteria: present, or above a cutoff level, in >50% of biological samples of the disease cells or tissue from individuals diagnosed with the disease state; absent, or below a cutoff level, in >95% of biological samples of the normal cells or tissue from individuals without the disease state; present, or above a cutoff level, in >50% of biological samples comprising cells, serum, blood, plasma, amniotic fluid, sputum, urine, bodily fluids, bodily secretions, bodily excretions, or fractions thereof, from individuals diagnosed with the disease state; absent, or below a cutoff level, in >95% of biological samples comprising cells, serum, blood, plasma, amniotic fluid, sputum, urine, bodily fluids, bodily secretions, bodily excretions, or fractions thereof, from individuals without the disease state; present with a z-value of >1.65 in the biological sample comprising cells, serum, blood, plasma, amniotic fluid, sputum, urine, bodily fluids, bodily secretions, bodily excretions, or fractions thereof, from individuals diagnosed with the disease state. At least 50% of the markers in a set each comprise one or more methylated residues, and/or at least 50% of the markers in a set that are present, or above a cutoff level, or present with a z-value of >1.65, comprise of one or more methylated residues, in the biological sample comprising cells, serum, blood, plasma, amniotic fluid, sputum, urine, bodily fluids, bodily secretions, bodily excretions, or fractions thereof, from at least 50% of individuals diagnosed with the disease state. The method involves obtaining a biological sample. The biological sample includes cell-free DNA, RNA, and/or protein originating from the cells or tissue and from one or more other tissues or cells, and the biological sample is selected from the group consisting of cells, serum, blood, plasma, amniotic fluid, sputum, urine, bodily fluids, bodily secretions, and bodily excretions, or fractions thereof. The sample is fractionated into one or more fractions, wherein at least one fraction comprises exosomes, tumor-associated vesicles, other protected states, or cell-free DNA, RNA, and/or protein. Nucleic acid molecules in the one or more fractions are subjected to a bisulfite treatment under conditions suitable to convert unmethylated cytosine residues to uracil residues. At least two enrichment steps are carried out for 50% or more disease-specific and/or cell/tissue-specific DNA, RNA, and/or protein markers during either said fractionating and/or by carrying out a nucleic acid amplification step. The method further involves performing one or more assays to detect and distinguish the plurality of disease-specific and/or cell/tissue-specific DNA, RNA, and/or protein markers, thereby identifying their presence or levels in the sample, wherein individuals are diagnosed or prognosed with the disease state if a minimum of 2 or 3 markers are present or are above a cutoff level in a marker set comprising from 6-12 markers; or a minimum of 3, 4, or 5 markers are present or are above a cutoff level in a marker set comprising from 12-24 markers; or a minimum of 3, 4, 5, or 6 markers are present or are above a cutoff level in a marker set comprising from 24-36 markers; or a minimum of 4, 5, 6, 7, or 8 markers are present or are above a cutoff level in a marker set comprising from 36-48 markers; or a minimum of 6, 7, 8, 9, 10, 11, or 12 markers are present or are above a cutoff level in a marker set comprising from 48-72 markers, or a minimum of 7, 8, 9, 10, 11, 12 or 13 markers are present or are above a cutoff level in a marker set comprising from 72-96 markers, or a minimum of 8, 9, 10, 11, 12, 13 or “n”/12 markers are present or are above a cutoff level in a marker set comprising 96−“n” markers, when “n”>168 markers.

Another aspect of the present application is directed to a method of diagnosing or prognosing a disease state of a solid tissue cancer including colorectal adenocarcinoma, stomach adenocarcinoma, esophageal carcinoma, breast lobular and ductal carcinoma, uterine corpus endometrial carcinoma, ovarian serous cystadenocarcinoma, cervical squamous cell carcinoma and adenocarcinoma, uterine carcinosarcoma, lung adenocarcinoma, lung squamous cell carcinoma, head & neck squamous cell carcinoma, prostate adenocarcinoma, invasive urothelial bladder cancer, liver hepatoceullular carcinoma, pancreatic ductal adenocarcinoma, or gallbladder adenocarcinoma based on identifying the presence or level of a plurality of disease-specific and/or cell/tissue-specific DNA, RNA, and/or protein markers in a biological sample of an individual. The plurality of markers is in a set comprising from 48-72 total cancer markers, 72-96 total cancer markers or ≥96 total cancer markers, wherein on average greater than one quarter such markers in a given set cover each of the aforementioned major cancers being tested. Each marker in a given set for a given solid tissue cancer is selected by having any one or more of the following criteria for that solid tissue cancer: present, or above a cutoff level, in >50% of biological samples of a given cancer tissue from individuals diagnosed with a given solid tissue cancer; absent, or below a cutoff level, in >95% of biological samples of the normal tissue from individuals without that given solid tissue cancer; present, or above a cutoff level, in >50% of biological samples comprising cells, serum, blood, plasma, amniotic fluid, sputum, urine, bodily fluids, bodily secretions, bodily excretions, or fractions thereof, from individuals diagnosed with a given solid tissue cancer; absent, or below a cutoff level, in >95% of biological samples comprising cells, serum, blood, plasma, amniotic fluid, sputum, urine, bodily fluids, bodily secretions, bodily excretions, or fractions thereof, from individuals without that given solid tissue cancer; present with a z-value of >1.65 in the biological sample comprising cells, serum, blood, plasma, amniotic fluid, sputum, urine, bodily fluids, bodily secretions, bodily excretions, or fractions thereof, from individuals diagnosed with a given solid tissue cancer. At least 50% of the markers in a set each comprise one or more methylated residues, and/or at least 50% of the markers in a set that are present, or above a cutoff level, or present with a z-value of >1.65, comprise of one or more methylated residues, in the biological sample comprising cells, serum, blood, plasma, amniotic fluid, sputum, urine, bodily fluids, bodily secretions, bodily excretions, or fractions thereof, from at least 50% of individuals diagnosed with a given solid tissue cancer. The method involves obtaining a biological sample including cell-free DNA, RNA, and/or protein originating from the cells or tissue and from one or more other tissues or cells, wherein the biological sample is selected from the group consisting of cells, serum, blood, plasma, amniotic fluid, sputum, urine, bodily fluids, bodily secretions, and bodily excretions, or fractions thereof. The sample is fractionated into one or more fractions, wherein at least one fraction comprises exosomes, tumor-associated vesicles, other protected states, or cell-free DNA, RNA, and/or protein. The nucleic acid molecules in the one or more fractions are subjected to a bisulfite treatment under conditions suitable to convert unmethylated cytosine residues to uracil residues. At least two enrichment steps are carried out for 50% or more of the given solid tissue cancer-specific and/or cell/tissue-specific DNA, RNA, and/or protein markers during either said fractionating and/or by carrying out a nucleic acid amplification step. The method further involves performing one or more assays to detect and distinguish the plurality of cancer-specific and/or cell/tissue-specific DNA, RNA, and/or protein markers, thereby identifying their presence or levels in the sample, wherein individuals are diagnosed or prognosed with the a solid-tissue cancer if a minimum of 4 markers are present or are above a cutoff level in a marker set comprising from 48-72 total cancer markers; or a minimum of 5 markers are present or are above a cutoff level in a marker set comprising from 72-96 total cancer markers; or a minimum of 6 or “n”/18 markers are present or are above a cutoff level in a marker set comprising 96 to “n” total cancer markers, when “n”>96 total cancer markers.

In accordance with this aspect of the present application, each marker in a given set for a given solid tissue cancer may be selected by having any one or more of the following criteria for that solid tissue cancer: present, or above a cutoff level, in >66% of biological samples of a given cancer tissue from individuals diagnosed with a given solid tissue cancer; absent, or below a cutoff level, in >95% of biological samples of the normal tissue from individuals without that given solid tissue cancer; present, or above a cutoff level, in >66% of biological samples comprising cells, serum, blood, plasma, amniotic fluid, sputum, urine, bodily fluids, bodily secretions, bodily excretions, or fractions thereof, from individuals diagnosed with a given solid tissue cancer; absent, or below a cutoff level, in >95% of biological samples comprising cells, serum, blood, plasma, amniotic fluid, sputum, urine, bodily fluids, bodily secretions, bodily excretions, or fractions thereof, from individuals without that given solid tissue cancer; present with a z-value of >1.65 in the biological sample comprising cells, serum, blood, plasma, amniotic fluid, sputum, urine, bodily fluids, bodily secretions, bodily excretions, or fractions thereof, from individuals diagnosed with a given solid tissue cancer.

Another aspect of the present application is directed to a method of diagnosing or prognosing a disease state of and identifying the most likely specific tissue(s) of origin of a solid tissue cancer in the following groups: Group 1 (colorectal adenocarcinoma, stomach adenocarcinoma, esophageal carcinoma); Group 2 (breast lobular and ductal carcinoma, uterine corpus endometrial carcinoma, ovarian serous cystadenocarcinoma, cervical squamous cell carcinoma and adenocarcinoma, uterine carcinosarcoma); Group 3 (lung adenocarcinoma, lung squamous cell carcinoma, head & neck squamous cell carcinoma); Group 4 (prostate adenocarcinoma, invasive urothelial bladder cancer); and/or Group 5 (liver hepatoceullular carcinoma, pancreatic ductal adenocarcinoma, or gallbladder adenocarcinoma) based on identifying the presence or level of a plurality of disease-specific and/or cell/tissue-specific DNA, RNA, and/or protein markers in a biological sample of an individual, wherein the plurality of markers is in a set comprising from 36-48 group-specific cancer markers, 48-64 group-specific cancer markers, or ≥64 group-specific cancer markers, wherein on average greater than one third of such markers in a given set cover each of the aforementioned cancers being tested within that group. Each marker in a given set for a given solid tissue cancer is selected by having any one or more of the following criteria for that solid tissue cancer: present, or above a cutoff level, in >50% of biological samples of a given cancer tissue from individuals diagnosed with a given solid tissue cancer; absent, or below a cutoff level, in >95% of biological samples of the normal tissue from individuals without that given solid tissue cancer; present, or above a cutoff level, in >50% of biological samples comprising cells, serum, blood, plasma, amniotic fluid, sputum, urine, bodily fluids, bodily secretions, bodily excretions, or fractions thereof, from individuals diagnosed with a given solid tissue cancer; absent, or below a cutoff level, in >95% of biological samples comprising cells, serum, blood, plasma, amniotic fluid, sputum, urine, bodily fluids, bodily secretions, bodily excretions, or fractions thereof, from individuals without that given solid tissue cancer; present with a z-value of >1.65 in the biological sample comprising cells, serum, blood, plasma, amniotic fluid, sputum, urine, bodily fluids, bodily secretions, bodily excretions, or fractions thereof, from individuals diagnosed with a given solid tissue cancer. At least 50% of the markers in a set each comprise one or more methylated residues, and/or at least 50% of the markers in a set that are present, or above a cutoff level, or present with a z-value of >1.65 comprise one or more methylated residues, in the biological sample comprising cells, serum, blood, plasma, amniotic fluid, sputum, urine, bodily fluids, bodily secretions, bodily excretions, or fractions thereof, from at least 50% of individuals diagnosed with a given solid tissue cancer. The method involves obtaining the biological sample. The biological sample includes cell-free DNA, RNA, and/or protein originating from the cells or tissue and from one or more other tissues or cells, wherein the biological sample is selected from the group consisting of cells, serum, blood, plasma, amniotic fluid, sputum, urine, bodily fluids, bodily secretions, and bodily excretions, or fractions thereof. The sample is fractionated into one or more fractions, wherein at least one fraction comprises exosomes, tumor-associated vesicles, other protected states, or cell-free DNA, RNA, and/or protein. The nucleic acid molecules in the one or more fractions are subjected to a bisulfite treatment under conditions suitable to convert unmethylated cytosine residues to uracil residues. At least two enrichment steps are carried out for 50% or more of the given solid tissue cancer-specific and/or cell/tissue-specific DNA, RNA, and/or protein markers during either said fractionating and/or by carrying out a nucleic acid amplification step. The method further involves performing one or more assays to detect and distinguish the plurality of cancer-specific and/or cell/tissue-specific DNA, RNA, and/or protein markers, thereby identifying their presence or levels in the sample, wherein individuals are diagnosed or prognosed with a solid-tissue cancer if a minimum of 4 markers are present or are above a cutoff level in a marker set comprising from 36-48 group-specific cancer markers; or a minimum of 5 markers are present or are above a cutoff level in a marker set comprising from 48-64 group-specific cancer markers; or a minimum of 6 or “n”/12 markers are present or are above a cutoff level in a marker set comprising 64 to “n” total cancer markers, when “n”>64 group-specific cancer markers.

In accordance with this aspect of the present application, each marker in a given set for a given solid tissue cancer may be selected by having any one or more of the following criteria for that solid tissue cancer: present, or above a cutoff level, in >66% of biological samples of a given cancer tissue from individuals diagnosed with a given solid tissue cancer; absent, or below a cutoff level, in >95% of biological samples of the normal tissue from individuals without that given solid tissue cancer; present, or above a cutoff level, in >66% of biological samples comprising cells, serum, blood, plasma, amniotic fluid, sputum, urine, bodily fluids, bodily secretions, bodily excretions, or fractions thereof, from individuals diagnosed with a given solid tissue cancer; absent, or below a cutoff level, in >95% of biological samples comprising cells, serum, blood, plasma, amniotic fluid, sputum, urine, bodily fluids, bodily secretions, bodily excretions, or fractions thereof, from individuals without that given solid tissue cancer; present with a z-value of >1.65 in the biological sample comprising cells, serum, blood, plasma, amniotic fluid, sputum, urine, bodily fluids, bodily secretions, bodily excretions, or fractions thereof, from individuals diagnosed with a given solid tissue cancer.

In one embodiment, the at least two enrichment steps comprise of two or more of the following steps: capturing or separating exosomes or extracellular vesicles or markers in other protected states; capturing or separating a platelet fraction; capturing or separating circulating tumor cells; capturing or separating RNA-containing complexes; capturing or separating cfDNA-nucleosome or differentially modified cfDNA-histone complexes; capturing or separating protein targets or protein target complexes; capturing or separating auto-antibodies; capturing or separating cytokines; capturing or separating methylated cfDNA; capturing or separating marker specific DNA, cDNA, miRNA, lncRNA, ncRNA, or mRNA, or amplified complements, by hybridization to complementary capture probes in solution, on magnetic beads, or on a microarray; amplifying miRNA markers, non-coding RNA markers (lncRNA & ncRNA markers), mRNA markers, exon markers, splice-variant markers, translocation markers, or copy number variation markers in a linear or exponential manner via a polymerase extension reaction, polymerase chain reaction, bisulfite-methyl-specific polymerase chain reaction, reverse-transcription reaction, bisulfite-methyl-specific ligation reaction, and/or ligation reaction, using DNA polymerase, reverse transcriptase, DNA ligase, RNA ligase, DNA repair enzyme, RNase, RNaseH2, endonuclease, restriction endonuclease, exonuclease, CRISPR, DNA glycosylase or combinations thereof; selectively amplifying one or more target regions containing mutation markers or bisulfite-converted DNA methylation markers, while suppressing amplification of the target regions containing wild-type sequence or bisulfite-converted unmethylated sequence or complement sequence thereof, in a linear or exponential manner via a polymerase extension reaction, polymerase chain reaction, bisulfite-methyl-specific polymerase chain reaction, reverse-transcription reaction, bisulfite-methyl-specific ligation reaction, and/or ligation reaction, using DNA polymerase, reverse transcriptase, DNA ligase, RNA ligase, DNA repair enzyme, RNase, RNaseH2, endonuclease, restriction endonuclease, exonuclease, CRISPR, DNA glycosylase or combinations thereof; preferentially extending, ligating, or amplifying one or more primers or probes whose 3′-OH end has been liberated in an enzyme and sequence-dependent process; using one or more blocking oligonucleotide primers comprising one or more mismatched bases at the 3′ end or comprising one or more nucleotide analogs and a blocking group at the 3′ end under conditions that interfere with polymerase extension or ligation during said reaction of target-specific primer or probes hybridized in a base-specific manner to wildtype sequence or bisulfite-converted unmethylated sequence or complement sequence thereof.

In certain embodiment, the one or more assays to detect and distinguish the plurality of disease-specific and/or cell/tissue-specific DNA, RNA, or protein markers comprise one or more of the following: a quantitative real-time PCR method (qPCR); a reverse transcriptase-polymerase chain reaction (RTPCR) method; a bisulfite qPCR method; a digital PCR method (dPCR); a bisulfite dPCR method; a ligation detection method, a ligase chain reaction, a restriction endonuclease cleavage method; a DNA or RNA nuclease cleavage method; a micro-array hybridization method; a peptide-array binding method; an antibody-array method; a mass spectrometry method; a liquid chromatography-tandem mass spectrometry (LC-MS/MS) method; a capillary or gel electrophoresis method; a chemiluminescence method; a fluorescence method; a DNA sequencing method; a bisulfite conversion-DNA sequencing method; an RNA sequencing method; a proximity ligation method; a proximity PCR method; a method comprising immobilizing an antibody-target complex; a method comprising immobilizing an aptamer-target complex; an immunoassay method; a method comprising a Western blot assay; a method comprising an enzyme linked immunosorbent assay (ELISA); a method comprising a high-throughput microarray-based enzyme-linked immunosorbent assay (ELISA); or a method comprising a high-throughput flow-cytometry-based enzyme-linked immunosorbent assay (ELISA).

In certain embodiments, the one or more cutoff levels of the one or more assays to detect and distinguish the plurality of disease-specific and/or cell/tissue-specific DNA, RNA, or protein markers comprise one or more of the following calculations, comparisons, or determinations, in the one or more marker assays comparing samples from the disease vs. normal individual: marker ΔCt value is >2; marker ΔCt value is >4; ratio of detected marker-specific signal is >1.5; ratio of detected marker-specific signal is >3; ratio of marker concentrations is >1.5; ratio of marker concentrations is >3; enumerated marker-specific signals differ by >20%; enumerated marker-specific signals differ by >50%; marker-specific signal from a given disease sample is >85%; >90%; >95%; >96%; >97%; or >98% of the same marker-specific signals from a set of normal samples; or marker-specific signal from a given disease sample has a z-score of >1.03; >1.28; >1.65; >1.75; >1.88; or >2.05 compared to the same marker-specific signals from a set of normal samples.

Another aspect of the present application relates to a two-step method of diagnosing or prognosing a disease state of cells or tissue based on identifying the presence or level of a plurality of disease-specific and/or cell/tissue-specific DNA, RNA, and/or protein markers in a biological sample of an individual. The method involves obtaining a biological sample, the biological sample including exosomes, tumor-associated vesicles, markers within other protected states, cell-free DNA, RNA, and/or protein originating from the cells or tissue and from one or more other tissues or cells, wherein the biological sample is selected from the group consisting of cells, serum, blood, plasma, amniotic fluid, sputum, urine, bodily fluids, bodily secretions, and bodily excretions, or fractions thereof. A first step is applied to the biological samples with an overall sensitivity of >80% and an overall specificity of >90% or an overall Z-score of >1.28 to identify individuals more likely to be diagnosed or prognosed with the disease state. A second step is then applied to biological samples from those individuals identified in the first step with an overall specificity of >95% or an overall Z-score of >1.65 to diagnose or prognose individuals with the disease state. The first step and the second step are carried out using a method of the present application. The first step uses markers to cover many cancers, where the aim is to obtain high sensitivity for early cancers where the number of marker molecules in the blood may be limited. The second step then would score for additional markers to verify that the initial result was a true positive, as well as to identify the likely tissue of origin. The second step may include the methods described herein, and/or additional methods such as next-generation sequencing.

Fluorescent Labeling.

Consider an instrument that can detect 5 fluorescent signals, F1, F2, F3, F4, and F5, respectively. As an example, in the case of colon cancer, the highest frequency mutations will be found for K-ras, p53, APC and BRAF. Mutations in these four genes could be detected with a single fluorescent signal; F1, F2, F3, F4. If the scale is 1000 FU, then primer would be added using ratios of labeled and unlabeled UniTaq primers, such that amplification of LDR products on mutant target of these genes yields about 300 FU at the plateau. For the controls, the F5 would be calibrated to give a signal of 100 FU for a 1:1,000 dilution quantification control, and an additional 300 FU for ligation of mutant probe on wild-type control (should give no or low background signal).

For the other genes commonly mutated in colon cancer as shown below, (or even lower abundance mutations in the p53 gene) the following coding system may be used: Two fluorescent signals in equimolar amount at the 5′ end of the same UniTaq, with unlabeled primer titrated in, such that both fluorescent signals plateau at 100 FU. If fluorescent signals are F1, F2, F3, F4, then that gives the ability to detect mutations in 4 genes using a single fluorescent signal, and in mutations in 6 genes using combinations of fluorescent signal:

| | |
|---|---|
| Gene 1 = F1 (300 FU) | (p53, Hot Spots) |
| Gene 2 = F2 (300 FU) | (KRAS) |
| Gene 3 = F3 (300 FU) | (APC) |
| Gene 4 = F4 (300 FU) | (BRAF) |
| Gene 5 = F1 (100 FU), F2 (100 FU) | (PIK3CA) |
| Gene 6 = F1 (100 FU), F3 (100 FU) | (FBXW7) |
| Gene 7 = F1 (100 FU), F4 (100 FU) | (SMAD4) |
| Gene 8 = F2 (100 FU), F3 (100 FU) | (p53, additional) |
| Gene 9 = F2 (100 FU), F4 (100 FU) | (CTNNB1) |
| Gene 10 = F3 (100 FU), F4 (100 FU) | (NRAS) |

Suppose there is a second mutation, combined with a mutation in one of the top genes. This is easy to distinguish, since the top gene will always give more signal, independent if it is overlapping with the other fluorescent signals or not. For example, if the fluorescent signal is F1 100 FU, and F2 400 FU, that would correspond to mutations in Gene 2 and Gene 5.

If there are two mutations from the less commonly mutated genes (Gene 5-Gene 10) then the results will appear either as an overlap in fluorescent signals, i.e. F1 200 FU, F2 100 FU, F4 100 FU, or all 4 fluorescent signals. If the fluorescent signals are in the ratio of 2:1:1, then it is rather straightforward to figure out the 2 mutations: in the above example, F1 200 FU, F2 100 FU, F4 100 FU, would correspond to mutations in Gene 5 and Gene 7. A similar approach for multiplexing different colors has been described by the Kartalov group (Raj agopal et al., “Supercolor Coding Methods for Large-Scale Multiplexing of Biochemical Assays,” Anal. Chem. 85(16):7629-36 (2013); U.S. Patent Application Publication No. 20140213471A1, which are hereby incorporated by reference in their entirety).

More recently, digital droplet PCR (ddPCR) has been used to provide accurate quantification of the number of mutant or methylated molecules in a clinical sample. In general, amplification in a droplet implies at least a single molecule of the target was present in that droplet. Thus, when using a sufficient number of droplets that way exceed the number of initial targets, it is assumed that a given droplet only had a single molecule of the target. Thus, end-point PCR is often used to monitor the number of products.

Consider an instrument that can detect 5 fluorescent signals, F1, F2, F3, F4, and F5, respectively. Methylation in the promoter region of some genes often methylated in colon cancer could be used for the first four channels, for example F1=VIM, F2=SEPT9, F3=CLIP4, and F4=GSG1L. The last channel, F5 would be used as a control to assure a given droplet contained proper reagents, etc. Once again, combinations of fluorescent signal may be used to simultaneously detect methylation at 10 different promoter regions.

| | |
|---|---|
| Gene 1 = F1 | (VIM) |
| Gene 2 = F2 | (SEPT9) |
| Gene 3 = F3 | (CLIP4 |
| Gene 4 = F4 | (GSG1L) |
| Gene 5 = F1 + F2 | (PP1R16B) |
| Gene 6 = F1 + F3 | (KCNA3) |
| Gene 7 = F1 + F4 | (GDF6) |
| Gene 8 = F2 + F3 | (ZNF677) |
| Gene 9 = F2 + F4 | (CCNA1) |
| Gene 10 = F3 + F4 | (STK32B) |

For simplicity, consider how ddPCR may be used to accurately enumerate the number of original methylated molecules at 4 promoter regions using exPCR-ddPCR (see for example, FIGS. 4, 5, 6, 7, 8, 11, 12, 13, 14, 15, 18, 19, 20, 21 & 22). The approach also works using PCR-LDR-qPCR or exPCR-LDR-qPCR (see FIGS. 2, 3, 9, 10, 16 & 17). For this illustration, consider a total of 48 methylated regions are being detected, with 4 promoter regions in a single ddPCR reaction comprising 10,000 droplets or micro-pores or micro-wells. Consider a sample with 2, 4, 5, and 1 molecule(s) of methylated promoter regions for VIM, SEPT9, CLIP4, and GSG1L, respectively. Assume the initial one-sided primer extension with blocking primer has an efficiency of 50%, so after 20 cycles, there are=20; 40; 50; and 10 extension products of methylated promoter regions for VIM, SEPT9, CLIP4, and GSG1L, respectively. Also, with blocking primer for the top strand, again, assuming a general efficiency of 50%, after 10 cycles of PCR, there are (1.5 to the 10^{th}=57×number of initial extension products)=1,140; 2,280; 2,850; and 570 copies of the PCR products for methylated VIM, SEPT9, CLIP4, and GSG1L respectively. When such products are then diluted into 12 ddPCR reactions, on average, a given chamber will comprise of 95; 190; 237; and 48 copies of the PCR products for methylated VIM, SEPT9, CLIP4, and GSG1L, respectively. This is a total of about 570 of molecules that would be amplified with primers for the total PCR products for methylated VIM, SEPT9, CLIP4, and GSG1L. If the ddPCR comprises 10,000 droplets or micro-pores or micro-wells, on average, only 1 in 20 will actually comprise a PCR reaction; the chances of a given droplet having two amplicons that would compete with each other for resources would be about 1 in 400, or about 25 droplets would comprise 2 amplicons, which would be only 5% of the total number of droplets with only a single amplicon. Since there are 6 combinations of 2 different amplicons, on average, less than 2% of the droplets would contain two amplicons. In other words, the rare droplet comprising 2 or 3 or 4 colors would not need to be de-convoluted, they could simply be ignored as they represent approximately 4-6 droplets compared to about 48 droplets arising from a single molecule in the original sample. While it may be a bit difficult to distinguish 190 from 237 droplets, i.e. starting with 4 or 5 molecules of a given methylated target, it should be relatively straightforward to distinguish 95; 190; and 48 copies, corresponding to 2, 4, and 1 target molecules in the original sample.

For distinguishing and enumerating 10 methylation markers simultaneously in a single ddPCR reaction, consider a total of 50 methylated regions are being detected, with 10 promoter regions in a single ddPCR reaction comprising 10,000 droplets or micro-pores or micro-wells. Consider a sample with 2, 4, 5, 1, 0, 1, 3, 2, 0, and 1 molecule(s) of methylated promoter regions for VIM, SEPT9, CLIP4, GSG1L, PP1R16B, KCNA3, GDF6, ZNF677, CCNA1, and STK32B, respectively. Assume the initial one-sided primer extension with blocking primer has an efficiency of 50%, so after 20 cycles, there are=20; 40; 50; 10; 0; 10; 30; 20; 0; and 10 extension products of methylated promoter regions for VIM, SEPT9, CLIP4, GSG1L, PP1R16B, KCNA3, GDF6, ZNF677, CCNA1, and STK32B, respectively. Also, with blocking primer for the top strand, again, assuming a general efficiency of 50%, after 6 cycles of PCR, there are (1.5 to the 6^{th}=11×number of initial extension products)=220; 440; 550; 110; 0; 110; 330; 220; 0; and 110 copies of the PCR products for methylated VIM, SEPT9, CLIP4, GSG1L, PP1R16B, KCNA3, GDF6, ZNF677, CCNA1, and STK32B, respectively. When such products are then diluted into 5 ddPCR reactions, on average, a given chamber will comprise of 44; 88; 110; 22; 0; 22; 66; 44; 0; and 22 copies of the PCR products for methylated VIM, SEPT9, CLIP4, GSG1L, PP1R16B, KCNA3, GDF6, ZNF677, CCNA1, and STK32B, respectively. This is a total of about 418 of molecules that would be amplified with primers for the total PCR products for methylated VIM, SEPT9, CLIP4, GSG1L, PP1R16B, KCNA3, GDF6, ZNF677, CCNA1, and STK32B. If the ddPCR comprises 10,000 droplets or micro-pores or micro-wells, on average, only 1 in 25 will actually comprise a PCR reaction; the chances of a given droplet having two amplicons that would compete with each other for resources would be about 1 in 625, or about 16 droplets would comprise 2 amplicons, which would be only 4% of the total number of droplets with only a single amplicon. Since there are 45 combinations of 2 different amplicons, on average, less than 0.1% of the droplets would contain a given two amplicons. In other words, the rare droplet comprising 2 or 3 or 4 colors would not need to be de-convoluted, they could simply be ignored as they represent one or two droplets compared to about 22 droplets arising from a single molecule in the original sample. While it may be a bit difficult to distinguish 88 from 110 droplets, i.e. starting with 4 or 5 molecules of a given methylated target, it should be relatively straightforward to distinguish 44, 88, and 22 copies, corresponding to 2, 4, and 1 target molecules in the original sample.

The above approach would also work for accurately enumerating mRNA, miRNA, ncRNA, or lncRNA target molecules. Instead of aliquoting the sample as described in step B of FIGS. 23-32, the sample is used directly for subsequent ddPCR enumeration. For distinguishing and enumerating 10 mRNA, ncRNA, or lncRNA markers simultaneously in a single ddPCR reaction, consider a total of 50 mRNA, ncRNA, or lncRNA regions are being detected in a single ddPCR reaction comprising 10,000 droplets or micro-pores or micro-wells. Once again, combinations of fluorescent signal may be used to simultaneously detect 10 mRNA or lncRNA markers.

| | |
|---|---|
| Gene 1 = F1 | (mRNA1) |
| Gene 2 = F2 | (mRNA2) |
| Gene 3 = F3 | (mRNA3) |
| Gene 4 = F4 | (mRNA4) |
| Gene 5 = F1 + F2 | (ncRNA5) |
| Gene 6 = F1 + F3 | (ncRNA6) |
| Gene 7 = F1 + F4 | (ncRNA7) |
| Gene 8 = F2 + F3 | (ncRNA8) |
| Gene 9 = F2 + F4 | (ncRNA9) |
| Gene 10 = F3 + F4 | (ncRNA10) |

Consider a sample with 2, 4, 15, 1, 0, 10, 3, 20, 0, and 1 molecule(s) of mRNA1-4 and ncRNA5-10, respectively, using essentially the RT-PCR-PCR-qPCR example illustrated in FIG. 28. Six cycles of RT-PCR will generate 64 cDNA copies of each transcript generating=128; 256; 960; 64; 0; 640; 192; 1280; 0; and 64 copies of mRNA1-4 and ncRNA5-10, respectively. When such products are then diluted into 5 ddPCR reactions, on average, a given chamber will comprise of 25; 51; 192; 13; 0; 128; 28; 256; 0; and 13 copies of the PCR products for mRNA1-4 and ncRNA5-10, respectively. This is a total of about 706 of molecules that would be amplified with primers for the total PCR products for methylated mRNA1-4 and ncRNA5-10. If the ddPCR comprises 10,000 droplets or micro-pores or micro-wells, on average, only 1 in 14 will actually comprise a PCR reaction. The two most common RNA's in this example; mRNA 3 and ncRNA5 would be present on average of 1 in 52 and 1 in 39, thus the chances of a given droplet having these two amplicons that would compete with each other for resources would be about 1 in 2028, or about 5 droplets would comprise 2 amplicons, which is still less than for a single molecule after amplification—which will generate 13 copies. In other words, the rare droplet comprising 2 or 3 or 4 colors would not need to be de-convoluted, they could simply be ignored as they represent from 1 to 5 droplets compared to at least 13 droplets arising from a single molecule in the original sample. If some RNA molecules are present in higher amounts, one can still de-convolute multiple signals arising from 2 amplicons in a given droplet, using the same approach of different color probes at different levels of FU (i.e. 300 FU for products with a single color; 100 FU each for products using 2 colors) as articulated earlier.

Another aspect of the present application relates to the ability to distinguish cancer at the earliest stages when analyzing markers within a blood sample. The average body contains about 6 liters (6,000 ml) of blood. A 10 ml sample will then comprise 1/600th of the sample. While some cancers (i.e. lung cancer, melanoma) have a high mutational load, other cancers (i.e. breast, ovarian) have few mutations, and even fewer at the earliest stages. In contrast, methylation changes in promoter regions (i.e. methylation markers) appear to be early events. For the purposes of the calculations below, assume that if a marker is present in the sample, it can be detected down to the single molecule level, independent of the technology that is being used to identify the marker.

On a practical level, different cancers have different frequencies for different mutational markers. For example, the mutation rate for gene K-ras is ˜30% and >90% for colorectal cancer and pancreatic cancer, respectively. While p53 is found mutated in about 50% of all cancers, more often than not, such a mutation is manifested in late-stage tumors. As a benchmark, a given cancer during its earliest stage, generates at least one detectable mutation. Suppose that at any given time, there are 200 mutated molecules circulating in the plasma of the patient. Given the total volume, if there is a 10 ml sample, taken, then there is about a ⅓^{rd} chance that the sample will contain at least 1 mutated molecule. A more accurate prediction would be based on the Poisson distribution. If there are 200 objects (i.e. mutated molecules) distributed into 600 bins (i.e. 600 aliquots of 10 ml representing the total blood volume of a patient), Poisson calculation would indicate that: 72% of wells will have 0 objects, 23.7% will have 1 object, 3.9% will have 2 objects, 0.4% will have 3 objects, etc. In other words, 28.1% of the aliquots would have at least one mutated molecule. If the assay is capable of detecting every single mutated molecule, then its sensitivity would be 28.1%. Likewise, if there were 300 objects (i.e. mutated molecules) distributed into 600 bins (i.e. 600 aliquots of 10 ml), then: 61% of wells will have 0 objects, 30.3% will have 1 object, 7.6% will have 2 objects, 1.3% will have 3 objects, etc. In other words, 39.4% of the aliquots would have at least one mutated molecule. If the assay is capable of detecting every single mutated molecule, its sensitivity is at 39.4%. Likewise, if there were 400 objects (i.e. mutated molecules) distributed into 600 bins (i.e. 600 aliquots of 10 ml), then: 51% of wells will have 0 objects, 34.3% will have 1 object, 11.5% will have 2 objects, 2.5% will have 3 objects, etc. In other words, 49% of the aliquots would have at least one mutated molecule. If the assay detects every single mutated molecule, its sensitivity would be 49%. Likewise, if there were 600 objects (i.e. mutated molecules) distributed into 600 bins (i.e. 600 aliquots of 10 ml), then the Poisson calculation would be: 36.8% of wells will have 0 objects, 36.8% will have 1 object, 18.3% will have 2 objects, 6.1% will have 3 objects, etc. In other words, 63.2% of the aliquots would have at least one mutated molecule. If the assay detects every single mutated molecule, then its sensitivity will be 63.2%. Nevertheless, on a practical level, even with a detectable marker load as high as 600 molecules, the assay would still miss 36.8% of early cancers for that type of tumor. Recent literature results have argued what constitutes “early cancer”, with some groups claiming stage I & II cancers are early cancer, while others claiming that stages I, II, and III cancers are early cancer, both the definition and type varies, but general when scoring form mutations the results have reported sensitivities ranging from around 20% to around 70%—which translates into missing 30% to 80% of early cancers (Klein et al., “Development of a Comprehensive Cell-free DNA (cfDNA) Assay for Early Detection of Multiple Tumor Types: The Circulating Cell-free Genome Atlas (CCGA) Study,” Journal of Clinical Oncology 36(15):12021-12021 (2018); Liu et al., “Breast Cancer Cell-free DNA (cfDNA) Profiles Reflect Underlying Tumor Biology: The Circulating Cell-free Genome Atlas (CCGA) Study,” Journal of Clinical Oncology 36(15):536-536 (2018), which are hereby incorporated by reference in their entirety).

The above calculations are performed based on the assumption that detecting even a single mutation is sufficient to call a patient positive. Initial work identifying mutations in the blood from patients with metastatic disease revealed an average of 5 mutations not only in the patients, but also in age-matched controls (Razavi, et al., “Cell-free DNA (cfDNA) Mutations From Clonal Hematopoiesis: Implications for Interpretation of Liquid Biopsy Tests,” Journal of Clinical Oncology 35(15): 11526-11526 (2017), which is hereby incorporated by reference in its entirety). This phenomenon, known as clonal hematopoiesis, results from accumulation of mutations in white-blood cells, that then undergo clonal expansion. Once the presence of such mutations are accounted for (by sequencing an aliquot of WBC DNA from the same individual), the accuracy or specificity of these tests has been set at 98%. For some cancers like ovarian cancer, which exhibit low mutation load, an estimated 60% of the disease at its early stage would be missed. To put these number in perspective, there were 20,240 new cases of ovarian cancer in the U.S. in 2018. Thus, about 55 million women (over the age of 50) should be tested for the disease. Such test would identify 8,096 women with ovarian cancer. However, there would be about 1.1 million false-positives. The positive predictive value of such a test would be around 0.74%. In other words, only one in 136 women who tested positive would actually have ovarian cancer, the rest would be false-positives.

For a multi-marker test of the present application, two or more markers need to be deemed positive in order the overall screening result to be deemed positive. By increasing the total number of individual markers used, as well as the number of markers required to call the overall screening test positive, both sensitivity and specificity for detecting early cancer may be improved. The overall early cancer detection sensitivity is a function of the average number of each marker in the blood, the average number of markers positive, the minimum number of markers required to call the sample positive, and the total number of markers scored. For example, if the test uses 12 methylation markers, that on average are methylated in >50% of tumors for that cancer type, then on average, about 6 markers will be methylated for a given sample. If on average there are 600 methylated molecules in the blood for each marker, then on average a total of 600×600=3,600 objects (i.e. methylated molecules) are distributed into 600 bins (i.e. 600 aliquots of 10 ml). As an approximate calculation based on the Poisson calculation, the distribution would be: 0.2% of wells will have 0 objects, 1.5% will have 1 object, 4.5% will have 2 objects, 8.9% will have 3 objects, 13.3% will have 4 objects, 16.0% will have 5 objects, 16.0% will have 6 objects, 13.8% will have 7 objects, 10.3% will have 8 objects etc. Suppose that at least two markers need to be called positive. In this case, 1.7% (=0.2%+1.5%) of the aliquots with either 0 or 1 object (i.e. methylation markers) would be called negative. Thus, if a minimum of two markers are required to call the sample positive, then the sensitivity of the assay would be 100%−1.7%=98.3% sensitivity. Suppose that at least three markers need to be called positive. In this case, aliquots with either 0, 1 or 2 objects (i.e. methylation markers) would be called negative=0.2%+1.5%+4.5%=6.2%. Thus, if a minimum of two markers are required to call the sample positive, then the sensitivity of the assay would be 100%−6.2%=93.8% sensitivity. It is understood that a small fraction of aliquots with 3 markers positive will contain 2 molecules of one marker, and one molecule of a second marker, and thus not contain the minimum of three different markers positive, nevertheless, this is a small deviation from the approximate calculation above.

The overall early cancer detection specificity is a function of the average number of markers positive, the false-positive rate for each individual marker, the minimum number of markers required to call the sample positive, and the total number of markers scored. To estimate the overall false-positive rate, a formula is used based on the probability of binning different color socks into a number of drawers. Consider the percentage of false positives for each marker=“% FP”; the total number of markers “m”, and the minimum number of markers required to call the sample positive “n”. Then the formula for calculating overall false positive would be: (% FP)ⁿ×[m!/(m−n)!n!]. Suppose that percentage of false positives for each marker=“% FP” is at 4%; the total number of markers “m” is 12, and the minimum number of markers required to call the sample positive “n” is 3. Then the above formula for overall false-positives would be (4%)³×[12!/9!3!]=(4%)³×[12×11×10/6]=1.4%. Thus, the overall specificity would be [100%−1.4%]=98.6%. The actual individual false-positive rate may differ for different markers. Further, it may depend on the source of the false-positive signal. If for example, age-related methylation is due to clonal hematopoiesis, i.e. results from accumulation of methylations in white-blood cells, that then undergo clonal expansion. This type of false-positive may be mitigated by also scoring for methylation changes in white blood cells from the same patient. On the other hand, if the source of the false-positive signal is due to release of DNA into the plasma due to tissue inflammation, or for example breakdown of muscle tissue from weight-lifting, then mitigating that signal may require sampling the blood at a different time when the body is rested, or a month later when inflammation has subsided.

FIGS. 33, 34, 35, and 36 illustrate results for calculated overall Sensitivity and Specificity for 24, 36, 48, and 96 markers, respectively. These graphs are based on the assumption that the average individual marker sensitivity is 50%, and the average individual marker false-positive rate is from 2% to 5%. The sensitivity curves provide overall sensitivity as a function of the average number of molecules in the blood for each marker, with separate curves for each minimum number of markers needed to call a sample as positive. The specificity curves provide overall specificity as a function of individual marker false-positive rates, again with separate curves for each minimum number of markers needed to call a sample as positive. The calculated numbers for overall Sensitivity and Specificity for 12, 24, 36, 48, 72 and 96 markers, respectively are provided in the tables below.

TABLE 1
12 Markers Sensitivity;
Avg. Indiv. Mkr,: 50% Sensitivity

| Average | | | |
|---|---|---|---|
| Number of | | 12 markers, | 12 markers, |
| Molecules in | Mutation, 1 | Minimum 2 | Minimum 3 |
| Blood | Positive | Positive | Positive |
| 150 | 22.1% | 44.2% | 19.1% |
| 200 | 28.1% | 59.4% | 32.3% |
| 240 | 33.0% | 69.2% | 43.0% |
| 300 | 39.4% | 80.1% | 57.7% |
| 400 | 48.8% | 90.8% | 76.2% |
| 480 | 55.1% | 95.2% | 85.7% |
| 600 | 63.2% | 98.3% | 93.8% |

TABLE 2
12 Marker Specificity

| Individual | Minimum 2 | Minimum 3 |
|---|---|---|
| marker FP | Markers | Markers |
| rate | Positive | Positive |
| 2% | 97.4% | 99.9% |
| 3% | 94.1% | 99.5% |
| 4% | 89.4% | 98.6% |
| 5% | | 97.2% |

TABLE 3
24 Markers Sensitivity;
Avg. Indiv. Mkr,: 50% Sensitivity

| Average | | | | |
|---|---|---|---|---|
| Number of | | 24 markers, | 24 markers, | 24 markers, |
| Molecules in | Mutation, 1 | Minimum 3 | Minimum 4 | Minimum 5 |
| Blood | Positive | Positive | Positive | Positive |
| 150 | 22.1% | 57.7% | 35.3% | 18.5% |
| 200 | 28.1% | 76.2% | 56.7% | 37.1% |
| 240 | 33.0% | 85.7% | 70.6% | 52.4% |
| 300 | 39.4% | 93.8% | 84.9% | 71.5% |
| 400 | 48.8% | 98.6% | 95.8% | 90.0% |
| 480 | 55.1% | 99.6% | 98.6% | 96.2% |
| 600 | 63.2% | 99.9% | 99.8% | 99.2% |

TABLE 4
24 Marker Specificity

| Individual | Minimum 3 | Minimum 4 | Minimum 5 |
|---|---|---|---|
| marker FP | Markers | Markers | Markers |
| rate | Positive | Positive | Positive |
| 2% | 98.4% | 99.8% | 99.9% |
| 3% | 94.6% | 99.1% | 99.9% |
| 4% | 87.1% | 97.3% | 99.6% |
| 5% | | 93.4% | 98.7% |

TABLE 5
36 Marker Sensitivity;
Avg. Indiv. Mkr,: 50% Sensitivity

| Average | | | | | |
|---|---|---|---|---|---|
| Number of | | 36 markers, | 36 markers, | 36 markers, | 36 markers, |
| Molecules in | Mutation, 1 | Minimum 3 | Minimum 4 | Minimum 5 | Minimum 6 |
| Blood | Positive | Positive | Positive | Positive | Positive |
| 150 | 22.1% | 82.6% | 65.8% | 46.8% | 29.7% |
| 200 | 28.1% | 93.8% | 84.9% | 71.5% | 55.4% |
| 240 | 33.0% | 97.5% | 92.8% | 84.4% | 72.4% |
| 300 | 39.4% | 99.4% | 97.9% | 94.5% | 88.4% |
| 400 | 48.8% | 99.9% | 99.8% | 99.2% | 98.0% |
| 480 | 55.1% | 100.0% | 100.0% | 99.9% | 99.6% |
| 600 | 63.2% | 100.0% | 100.0% | 100.0% | 100.0% |

TABLE 6
36 Marker Specificity

| Individual | Minimum 3 | Minimum 4 | Minimum 5 | Minimum 6 |
|---|---|---|---|---|
| marker FP | Markers | Markers | Markers | Markers |
| rate | Positive | Positive | Positive | Positive |
| 2% | 94.3% | 99.1% | 99.9% | 100.0% |
| 3% | 80.7% | 95.2% | 99.1% | 99.9% |
| 4% | | 84.9% | 96.1% | 99.2% |
| 5% | | | 88.2% | 97.0% |

TABLE 7
48 Marker Sensitivity;
Avg. Indiv. Mkr,: 50% Sensitivity

| Average | | | | | | |
|---|---|---|---|---|---|---|
| Number of | | 48 markers, | 48 markers, | 48 markers, | 48 markers, | 48 markers, |
| Molecules | Mutation, 1 | Minimum 4 | Minimum 5 | Minimum 6 | Minimum 7 | Minimum 8 |
| in Blood | Positive | Positive | Positive | Positive | Positive | Positive |
| 150 | 22.1% | 84.9% | 71.6% | 55.6% | 39.6% | 25.8% |
| 200 | 28.1% | 95.8% | 90.1% | 80.9% | 68.7% | 54.8% |
| 240 | 33.0% | 99.1% | 97.2% | 93.4% | 87.1% | 78.1% |
| 300 | 39.4% | 99.8% | 99.3% | 98.1% | 95.6% | 92.3% |
| 400 | 48.8% | 99.9% | 99.9% | 99.8% | 99.7% | 99.1% |
| 480 | 55.1% | 99.9% | 99.9% | 99.9% | 99.9% | 99.9% |
| 600 | 63.2% | 99.9% | 99.9% | 99.9% | 99.9% | 99.9% |

TABLE 8
48 Marker Specificity

| Individual | Minimum 4 | Minimum 5 | Minimum 6 | Minimum 7 | Minimum 8 |
|---|---|---|---|---|---|
| marker FP | Markers | Markers | Markers | Markers | Markers |
| rate | Positive | Positive | Positive | Positive | Positive |
| 2% | 96.9% | 99.4% | 99.9% | 99.9% | 99.9% |
| 3% | 84.3% | 95.8% | 99.1% | 99.8% | 99.9% |
| 4% | | 82.5% | 95.0% | 98.8% | 99.8% |
| 5% | | | | 94.3% | 98.6% |

TABLE 9
72 Marker Sensitivity;
Avg. Indiv. Mkr,: 50% Sensitivity

| Average | | | | | | | | |
|---|---|---|---|---|---|---|---|---|
| Number of | | 72 markers, | 72 markers, | 72 markers, | 72 markers, | 72 markers, | 72 markers, | 72 markers, |
| Molecules | Mutation, 1 | Minimum 6 | Minimum 7 | Minimum 8 | Minimum 9 | Minimum 10 | Minimum 11 | Minimum 12 |
| in Blood | Positive | Positive | Positive | Positive | Positive | Positive | Positive | Positive |
| 150 | 22.1% | 88.4% | 79.3% | 67.6% | 54.4% | 41.3% | 29.4% | 19.7% |
| 200 | 28.1% | 98.0% | 95.4% | 91.0% | 84.5% | 75.8% | 65.3% | 53.8% |
| 240 | 33.0% | 99.6% | 98.9% | 97.5% | 94.9% | 90.8% | 84.9% | 77.2% |
| 300 | 39.4% | 99.9% | 99.9% | 99.7% | 99.3% | 98.5% | 97.0% | 94.5% |
| 400 | 48.8% | 99.9% | 99.9% | 99.9% | 99.9% | 99.9% | 99.9% | 99.7% |
| 480 | 55.1% | 99.9% | 99.9% | 99.9% | 99.9% | 99.9% | 99.9% | 99.9% |
| 600 | 63.2% | 99.9% | 99.9% | 99.9% | 99.9% | 99.9% | 99.9% | 99.9% |

TABLE 10
72 Marker Specificity

| Individual | Minimum | Minimum | Minimum | Minimum | Minimum | Minimum | Minimum |
|---|---|---|---|---|---|---|---|
| marker | 6 Markers | 7 Markers | 8 Markers | 9 Markers | 10 Markers | 11 Markers | 12 Markers |
| FP rate | Positive | Positive | Positive | Positive | Positive | Positive | Positive |
| 2% | 99.0% | 99.8% | 100.0% | 100.0% | 100.0% | 100.0% | 100.0% |
| 3% | 88.6% | 96.8% | 99.2% | 99.8% | 100.0% | 100.0% | 100.0% |
| 4% | | | 92.2% | 97.8% | 99.4% | 99.9% | 100.0% |
| 5% | | | | 83.4% | 94.8% | 98.5% | 99.6% |

TABLE 11
96 Marker Sensitivity;
Avg. Indiv. Mkr,: 50% Sensitivity

| Average | | | | | | | | |
|---|---|---|---|---|---|---|---|---|
| Number of | | 96 markers, | 96 markers, | 96 markers, | 96 markers, | 96 markers, | 96 markers, | 96 markers, |
| Molecules | Mutation, 1 | Minimum 7 | Minimum 8 | Minimum 9 | Minimum 10 | Minimum 11 | Minimum 12 | Minimum 13 |
| in Blood | Positive | Positive | Positive | Positive | Positive | Positive | Positive | Positive |
| 150 | 22.1% | 95.4% | 91.0% | 84.5% | 75.8% | 65.3% | 53.8% | 42.4% |
| 200 | 28.1% | 99.6% | 99.0% | 97.8% | 95.7% | 92.3% | 87.3% | 80.7% |
| 240 | 33.0% | 99.9% | 99.9% | 99.7% | 99.2% | 98.3% | 96.8% | 94.4% |
| 300 | 39.4% | 99.9% | 99.9% | 99.9% | 99.9% | 99.9% | 99.7% | 99.5% |
| 400 | 48.8% | 99.9% | 99.9% | 99.9% | 99.9% | 99.9% | 99.9% | 99.9% |
| 480 | 55.1% | 99.9% | 99.9% | 99.9% | 99.9% | 99.9% | 99.9% | 99.9% |
| 600 | 63.2% | 99.9% | 99.9% | 99.9% | 99.9% | 99.9% | 99.9% | 99.9% |

TABLE 12
96 Marker Specificity

| Individual | Minimum | Minimum | Minimum | Minimum | Minimum | Minimum | Minimum |
|---|---|---|---|---|---|---|---|
| marker | 7 Markers | 8 Markers | 9 Markers | 10 Markers | 11 Markers | 12 Markers | 13 Markers |
| FP rate | Positive | Positive | Positive | Positive | Positive | Positive | Positive |
| 2% | 98.5% | 99.7% | 99.9% | 100.0% | 100.0% | 100.0% | 100.0% |
| 3% | | 91.3% | 97.4% | 99.3% | 99.8% | 100.0% | 100.0% |
| 4% | | | | 88.2% | 96.3% | 99.0% | 99.7% |
| 5% | | | | | | 84.7% | 95.1% |

From the above tables, the receiver operating characteristic (ROC) curves may be calculated by plotting Sensitivity vs. 1-Specificity. Since these are theoretical calculations, the curves were generated for different levels of average marker false-positive rates of 2%, 3%, 4%, and 5%. To assist in visualizing the graphs and calculating the AUC (Area under curve), the edges were set at 100% and 0%, respectively. The ROC curves for 24 marker, 3% & 4% average marker false-positives, 36 marker, 3% & 4% average marker false-positives, and 48 marker, 2%, 3%, 4% & 5% average marker false-positives are provided in Table 13 below and for 48 Markers illustrated in FIGS. 37 and 38, respectively. Generally, the closer the AUC is to 1, the more accurate the test−values of >95% are desirable, and values >99% are superb. Using the benchmark of an average of 300 molecules in the blood for early cancer (Stage I & II), AUC values are at 95% with 24 markers, improve to 99% with 36 markers, and range from 98% to >99% with 48 markers, depending on average marker false-positive values. These graphs provide an illustration of the power of multiple marker assays for achieving good sensitivities and specificities.

TABLE 13
24, 36, & 48 Marker AUC Values from ROC Curves;
Avg. Indiv. Mkr,: 50% Sensitivity

| Total | | | | | | | |
|---|---|---|---|---|---|---|---|
| Markers: | | | | | | | |
| Individual | | | | | | | |
| marker | 150 | 200 | 240 | 300 | 400 | 480 | 600 |
| FP rate | Molecules | Molecules | Molecules | Molecules | Molecules | Molecules | Molecules |
| 24 Mkrs: 3% | 77% | 87% | | 96% | 99% | >99% | >99% |
| 24 Mkrs: 4% | 74% | 85% | | 95% | 99% | >99% | >99% |
| 36 Mkrs: 3% | 87% | 95% | 98% | 99% | >99% | >99% | >99% |
| 36 Mkrs: 4% | 78% | 89% | 95% | 98% | 99% | >99% | >99% |
| 48 Mkrs: 2% | 92% | 98% | 99% | >99% | >99% | >99% | >99% |
| 48 Mkrs: 3% | 89% | 97% | 99% | >99% | >99% | >99% | >99% |
| 48 Mkrs: 4% | 81% | 93% | 98% | 99% | >99% | >99% | >99% |
| 48 Mkrs: 5% | 71% | 86% | 94% | 98% | 99% | 99% | 99% |

How would the above markers work in a one-step cancer assay? To illustrate the challenges of developing an early cancer detection screen, consider the challenge of screening 107 million adults in the U.S. over the age of 50 for colorectal cancer—of which there are about 135,000 new cases that are diagnosed a year. In this example, if there is an average of 300 molecules in the blood for early cancer (Stage I & II), and taking the best-case scenario of individual marker FP rate is 2%, then if there is a 3-marker minimum, then overall FP rate is 1.6% for 24 markers, for a specificity of 98.4% (See FIG. 33B). At 3 markers, for Stage I & II cancer (at about 300 molecules of each positive marker in the blood), the test would miss 6.2%; i.e. for Stage I & II cancer the overall sensitivity would be 93.8% (See FIG. 33A), e.g. the test would correctly identify 93.8% of individuals with disease, which would be 126,630 individuals (out of 135,000 new cases). At a specificity of 98.4%, for 107 million individuals screened, the test would also generate 1.6%×107,000,000=1,712,000 false positives. Thus, the positive predictive value would be 126,630/(126,630+1,712,000)=around 6.8%, in other words, only one in 14 individuals who tested positive would actually have colorectal cancer, the rest would be false-positives.

However, if the individual marker FP rate is more realistic, say 4%, then more markers will be required to achieve better than 98% specificity, and this will be at the cost of sensitivity. If individual marker FP rate is 4%, then if there is a 5-marker minimum, then overall FP rate is 0.4% for 24 markers, for a specificity of 99.6% (See FIG. 33B). At 5 markers, for Stage I & II cancer (at about 300 molecules of each positive marker in the blood), the test would miss 28.5%; i.e. for Stage I & II cancer the overall sensitivity would be 71.5% (See FIG. 33A), e.g. the test would correctly identify 71.5% of individuals with disease, which would be 90,540 individuals (out of 135,000 new cases). At a specificity of 99.6%, for 107 million individuals screened, the test would also generate 0.4%×107,000,000=428,000 false positives. Thus, the positive predictive value would be 90,540/(90,540+428,000)=around 17.5%, in other words, only one in 5.7 individuals who tested positive would actually have colorectal cancer, the rest would be false-positives. A PPV of 17.5% is quite respectable, however, it would be achieved at the cost of missing 28.5% of early cancer.

Another aspect of the present application relates to a two-step method of diagnosing or prognosing a disease state of cells or tissue based on identifying the presence or level of a plurality of disease-specific and/or cell/tissue-specific DNA, RNA, and/or protein markers in a biological sample of an individual. The method involves obtaining a biological sample, the biological sample including exosomes, tumor-associated vesicles, markers within other protected states, cell-free DNA, RNA, and/or protein originating from the cells or tissue and from one or more other tissues or cells, wherein the biological sample is selected from the group consisting of cells, serum, blood, plasma, amniotic fluid, sputum, urine, bodily fluids, bodily secretions, and bodily excretions, or fractions thereof. A first step is applied to the biological samples with an overall sensitivity of >80% and an overall specificity of >85% or an overall Z-score of >1.03 to identify individuals more likely to be diagnosed or prognosed with the disease state. A second step is the applied to biological samples from those individuals identified in the first step with an overall specificity of >95% or an overall Z-score of >1.65 to diagnose or prognose individuals with the disease state. The first step and the second step are carried out using a method of the present application. The first step uses markers to cover many cancers, where the aim is to obtain high sensitivity for early cancers where the number of marker molecules in the blood may be limited. The second step then would score for additional markers to verify that the initial result was a true positive, as well as to identify the likely tissue of origin. The second step may include the methods described herein, and/or additional methods such as next-generation sequencing. The first step uses markers to cover many cancers, where the aim is to obtain high sensitivity for early cancers where the number of marker molecules in the blood may be limited. The second step then would score for additional markers to verify that the initial result was a true positive, as well as to identify the likely tissue of origin. The second step may include the methods described herein, and/or additional methods such as next-generation sequencing.

To illustrate one embodiment of how such a two-step cancer test may be designed, consider again the challenge of identifying patients with early colorectal cancer. In 2017, there were an estimated 95,520 new cases of colon cancer and 39,910 cases of rectal cancer diagnosed in the U.S.—for a total of about 135,000 new cases. Consider an initial test using 24 markers. In this example, if there is an average of 300 molecules in the blood for early cancer (Stage I & II), and if that would cover at least one mutation, then the sensitivity for identifying such a cancer by next generation sequencing would be 39.4% (See FIG. 33A). If the individual marker FP rate is 3%, then if there is a 3-marker minimum, then overall FP rate is 5.4% for 24 markers, for a specificity of 94.6% (See FIG. 33B). At 3 markers, for Stage I & II cancer (at about 300 molecules of each positive marker in the blood), the test would miss 6.2%; i.e. for Stage I & II cancer the overall sensitivity would be 93.8% (See FIG. 33A). Note that these levels of sensitivity and specificity are better than the current tests on the market. However, if the individual marker FP rate is 5%, then if there is a 4-marker minimum, then overall FP rate is 6.6% for 24 markers, for a specificity of 93.4% (See FIG. 33B). At 4 markers, for Stage I & II cancer (at about 300 molecules of each positive marker in the blood), the test would miss 15.1%; i.e. for Stage I & II cancer the sensitivity would be 84.9% (See FIG. 33A). These graphs illustrate a basic conflict of most diagnostic tests—improve the sensitivity of a test (i.e. less false-negatives), but sacrifice the test specificity (i.e. more false-positives), or improve the specificity of a test (less false-positives) at the risk of losing the test sensitivity (i.e. more false-negatives).

By using a two-step cancer test, the parameters may be adjusted to improve BOTH sensitivity and specificity. For example, the aforementioned 24 marker test, using 3 markers, for Stage I & II cancer (at about 300 molecules of each positive marker in the blood), the overall sensitivity would be 93.8%. Those samples that are scored as positives in the first step (24-markers specific to GI cancers)—including the false-positives would be retested in the second step with an expanded panel of 48 markers to provide coverage of colorectal cancers. If the individual marker FP rate is 3%, then if there is a 5-marker minimum, then overall FP rate is 4.2% for 48 markers, for a specificity of 95.8% (See FIG. 35B). At 5 markers, for Stage I & II cancer (at about 300 molecules of each positive marker in the blood), the test would miss 0.7%; i.e. for Stage I & 2 cancer the sensitivity would be 99.3% (See FIG. 35A). Technically, since the samples were already culled in the first step, the overall sensitivity is 93.8%×99.3%=93.1%. If the individual marker FP rate is 3%, then if there is a 6-marker minimum, then overall FP rate is <1% for 48 markers, for a specificity of 99.1% (See FIG. 35B). At 6 markers, for Stage I & II cancer (at about 300 molecules of each positive marker in the blood), the test would miss 1.9%; i.e. for Stage I & II cancer the sensitivity would be 98.1% (See FIG. 35A). Since the samples were already culled in the first step, the overall sensitivity is 93.8%×98.1%=92.0%. If the individual marker FP rate is 3%, then if there is a 7-marker minimum, then overall FP rate is <0.2% for 48 markers, for a specificity of 99.8% (See FIG. 35B). At 7 markers, for Stage I & II cancer (at about 300 molecules of each positive marker in the blood), the test would miss 4.4%; i.e. for Stage I & II cancer the sensitivity would be 95.6% (See FIG. 35A). Since the samples were already culled in the first step, the overall sensitivity is 93.8%×95.6%=89.7%.

Returning to the example of colorectal cancer, in particular the cases of microsatellite stable tumors (MSS) where the mutation load is low, for these calculations when relying on NGS sequencing alone (assuming 300 molecules with one mutation in the blood), an estimated 60% of early colorectal cancer would be missed. Again, to put these number in perspective, in the U.S., about 135,000 new cases of colorectal cancer are predicted in 2018. About 107 million individuals in the U.S. are over the age of 50 and should be tested for colorectal cancer. With the assumption of these samples containing at least 300 molecules with one mutation in the blood, such a test would find 54,000 men and women (out of 135,000 new cases) with colorectal cancer. However, with a specificity for sequencing at 98%, there would be about 2.1 million false-positives. The positive predictive value of such a test would be around 2.6%, in other words, only one in 39 individuals who tested positive would actually have colorectal cancer, the rest would be false-positives. In contrast, consider the two-step methylation marker test described above, wherein the first step has 24 methylation markers specific to GI cancers, while the second step has 48 methylation markers specific to colorectal cancer. In this example, as above, the calculations are done with the anticipation of an average of 300 methylated molecules per positive marker in the blood. Assuming individual marker false-positive rates of 3%, and with the first step requiring a minimum of 3 markers positive, then with an overall specificity of 94.6%, the first step would identify 5,778,000 individuals (out of 107,000,000 total adults over 50 in the U.S.) which would include at 93.8% sensitivity about 126,630 individuals with Stage I & II colorectal cancer (out of 135,000 total). However, those 5,778,000 presumptive positive individuals would be evaluated in the second step of 48 markers requiring a minimum of 6 markers positive, then the two-step test would identify 98.1%×93.8%=92.0%=124,200 individuals (out of 135,000 new cases) with colorectal cancer. With a specificity of 99.1%, the second test would also generate 5,778,000×0.9%=52,000 false-positives. The positive predictive value of such a test would be 124,200/176,200=70.5%, in other words, 2 in 3 individuals who tested positive would actually have colorectal cancer, an extraordinarily successful screen to focus on those patients who would most benefit from follow-up colonoscopy. The benefit in lives saved would be of incalculable value.

While the foregoing discussion has focused on methylation markers, with an average sensitivity of 50%, and individual marker false-positives ranging from 2%-5%, there are many other markers of cancer with varying sensitivities and specificities. In general, protein markers (with the exception of PSA and PSMA) have been of limited clinical utility for detection of early cancer because the false-positives are so high, resulting in very low positive predictive value. Cancer markers from bodily fluids (i.e. plasma, urine) include, but are not limited to plasma microRNAs (miRNA); mutations or methylation in cfDNA; exosomes with surface cancer-specific protein markers, or internal miRNA, ncRNA, lncRNA, mRNA, DNA; circulating cytokines, circulating proteins, or circulating antibodies against cancer-antigens; or nucleic-acid markers in whole blood (for review, see Nikolaou et al., “Systematic Review of Blood Diagnostic Markers in Colorectal Cancer,” Techniques in Coloproctology (2018), which is hereby incorporated by reference in its entirety). Several methods have been reported for detecting cancer-specific miRNAs in the serum or plasma of patients with colorectal (or others) cancers; these miRNAs include, but are not limited to: miR-1290; miR-21; miR-24; miR-320a; miR-423-5p; miR-29a; miR-125b; miR-17-3p; miR-92a; miR-19a; miR-19b; miR-15b; mir23a; miR-150; miR-223; miR-1229; miR-1246; miR-612; miR-1296; miR-933; miR-937; miR-1207; miR-31; miR-141; miR-224-3p; miR-576-5p; miR-885-5p; miR-200c; miR-203 (Imaoka et al., “Circulating MicroRNA-1290 as a Novel Diagnostic and Prognostic Biomarker in Human Colorectal Cancer,” Ann. Oncol. 27(10):1879-1886 (2016); Zhang et al., “Diagnostic and Prognostic Value of MicroRNA-21 in Colorectal Cancer: an Original Study and Individual Participant Data Meta-Analysis,” Cancer Epidemiol. Biomark. Prev. 23(12):2783-2792 (2016); Fang et al., “Plasma Levels of MicroRNA-24, MicroRNA-320a, and Micro-RNA-423-5p are Potential Biomarkers for Colorectal Carcinoma,” J. Exp. Clin. Cancer Res. 34:86 (2015); Toiyama et al., “MicroRNAs as Potential Liquid Biopsy Biomarkers in Colorectal Cancer: A Systematic Review,” Biochim. Biophys. Acta. pii: 50304-419X(18)30067-2 (2018); Nagy et al., “Comparison of Circulating miRNAs Expression Alterations in Matched Tissue and Plasma Samples During Colorectal Cancer Progression,” Pathol. Oncol. Res. doi: 10.1007/s12253-017-0308-1 (2017); Wang et al., “Novel Circulating MicroRNAs Expression Profile in Colon Cancer: a Pilot Study,” Eur. J. Med. Res. 22(1):51 (2017); U.S. Pat. No. 9,689,036 to Getts, et al.; U.S. Pat. No. 9,708,643 to Duttagupta, et al.; U.S. Pat. No. 9,868,992 to Goel, et al; U.S. Pat. No. 9,926,603 to Sozzi et al., which are hereby incorporated by reference in their entirety). Additional approaches for detecting low abundance miRNA are described in WO2016057832A2, which is hereby incorporated by reference in its entirety, or using other suitable means known in the art. FIG. 39 provides a list of blood-based, colon cancer-specific microRNA markers derived through analysis of TCGA microRNA datasets, which may be present in exosomes, tumor-associated vesicles, Argonaute complexes, or other protected states in the blood.

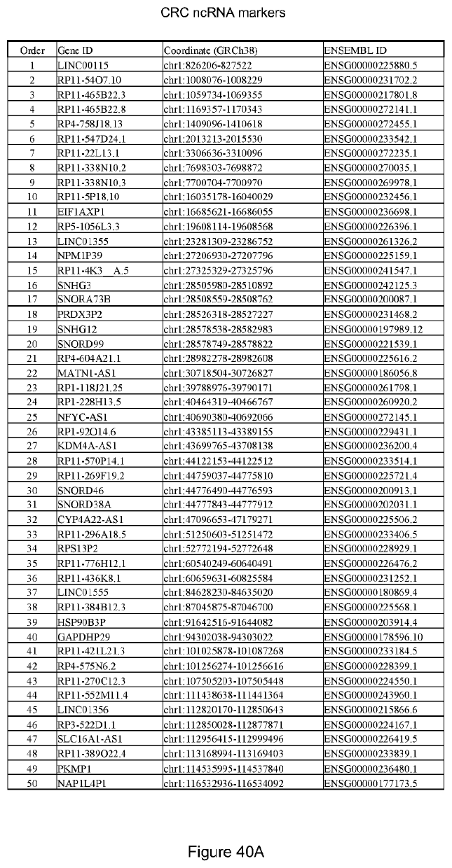

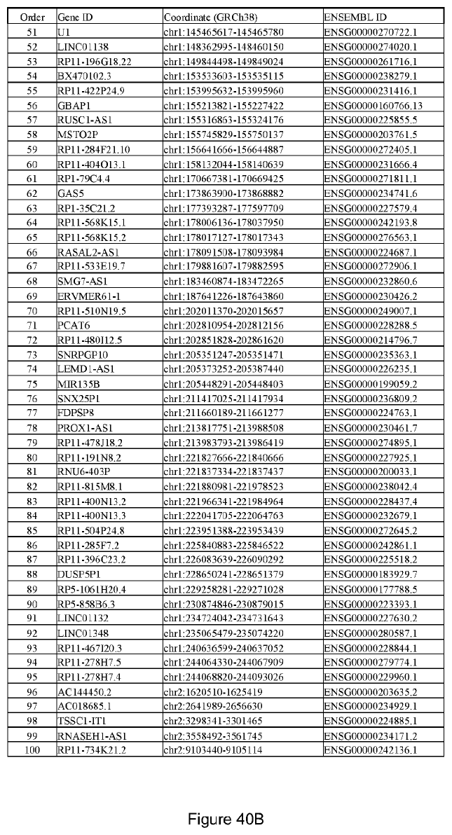

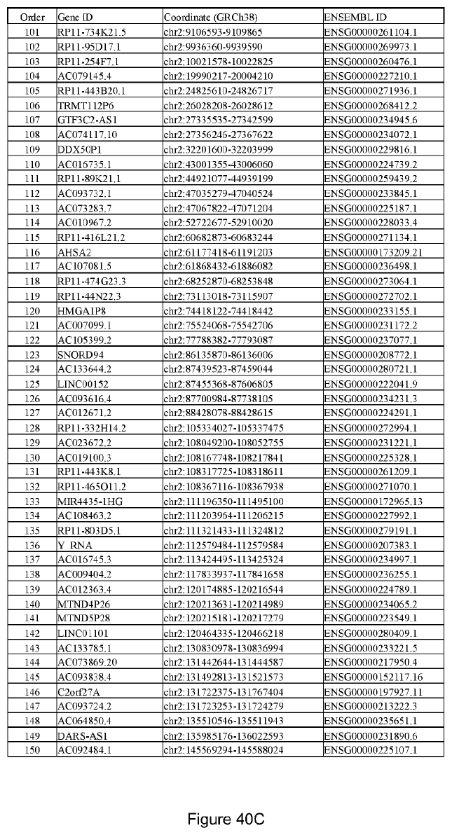

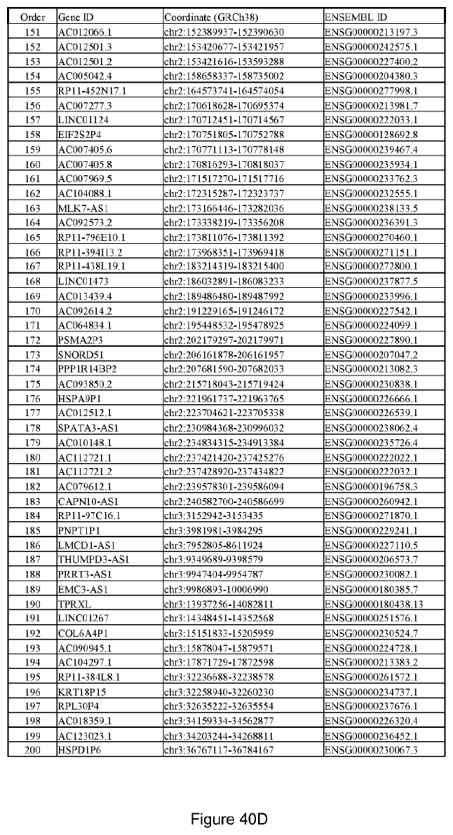

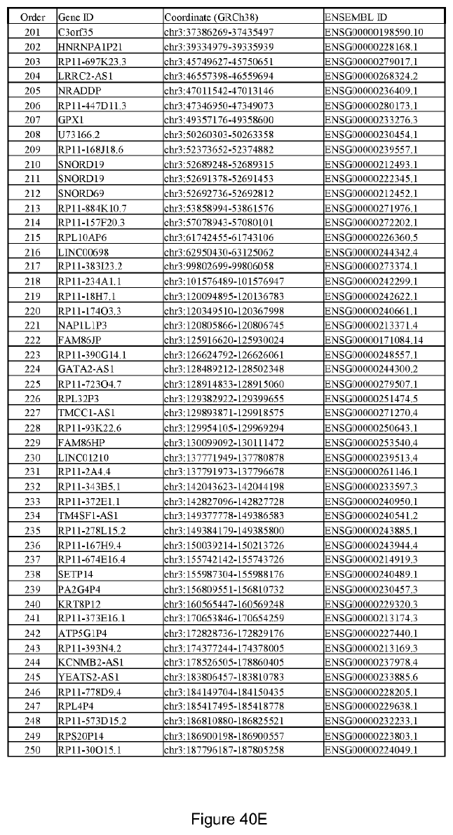

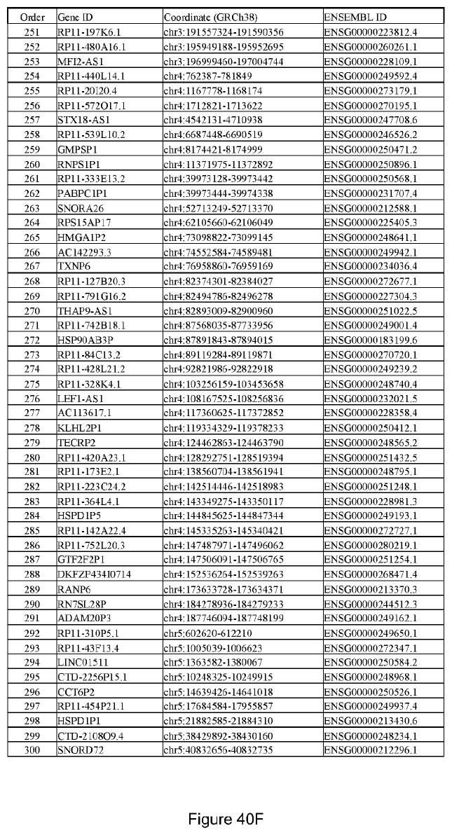

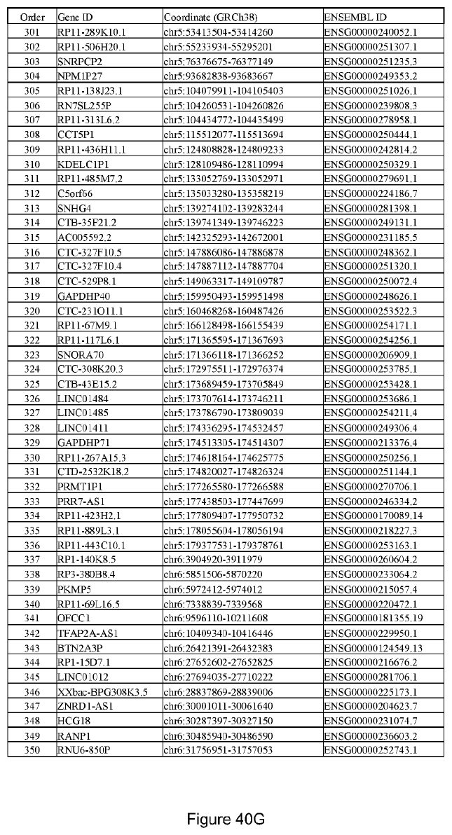

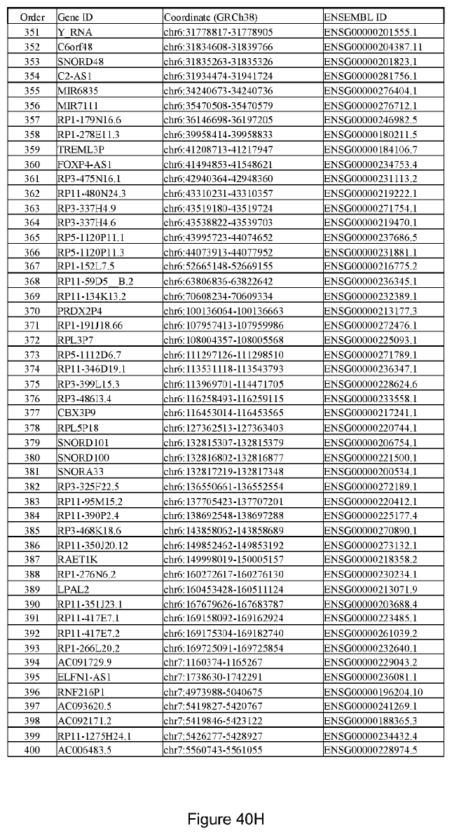

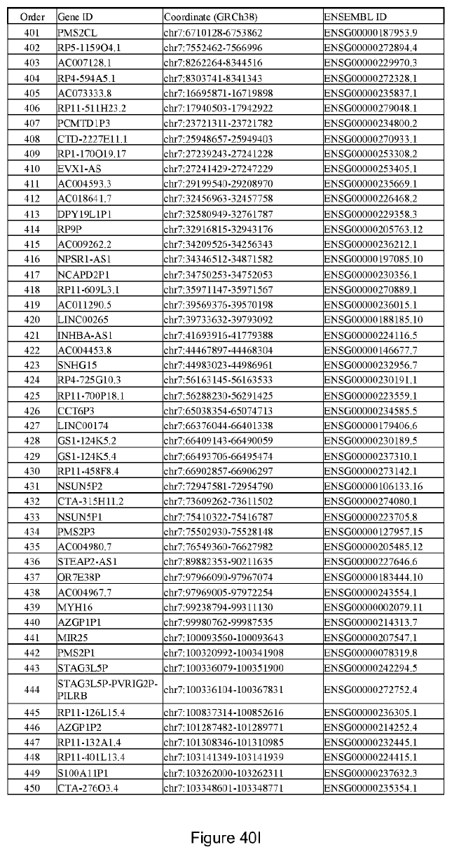

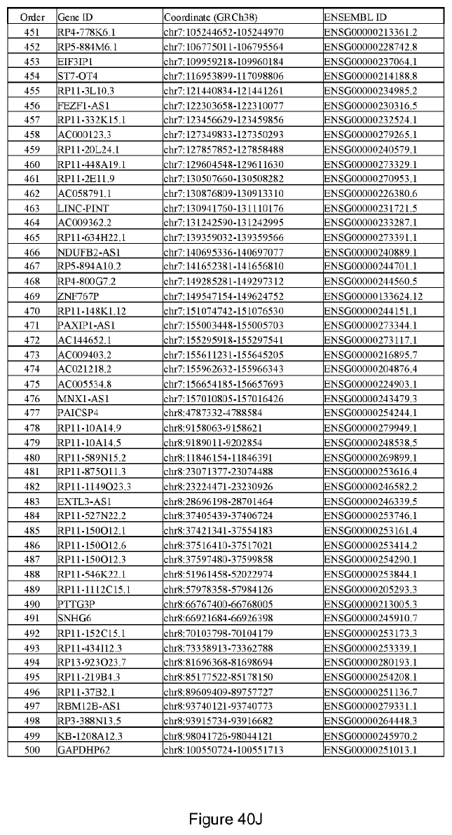

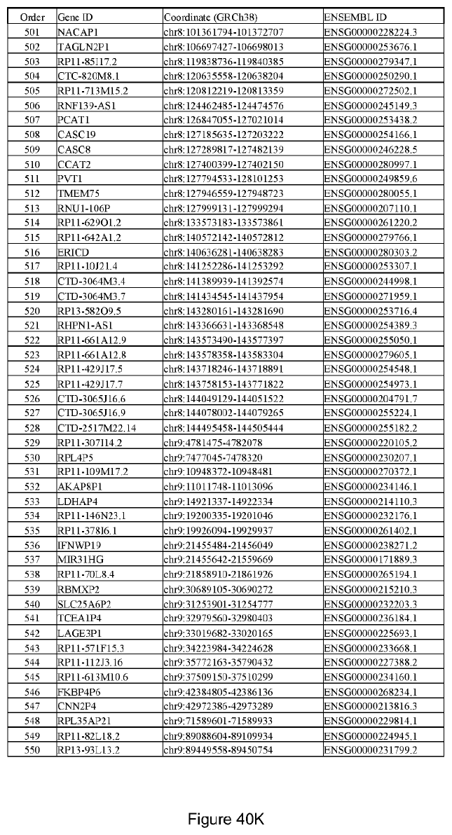

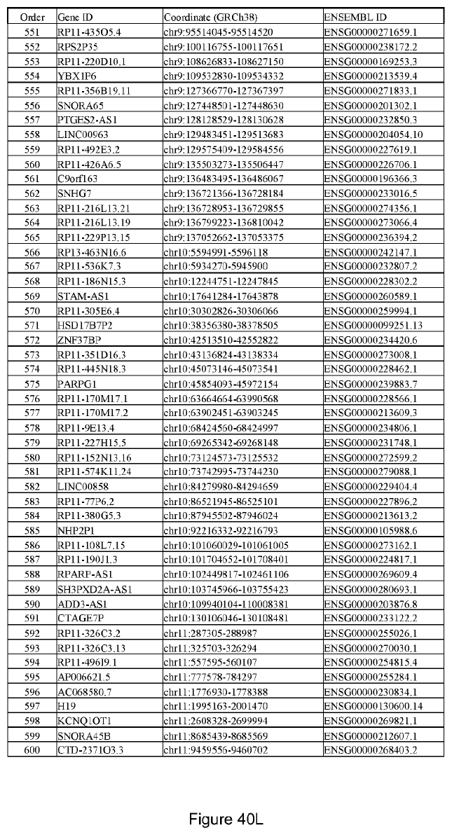

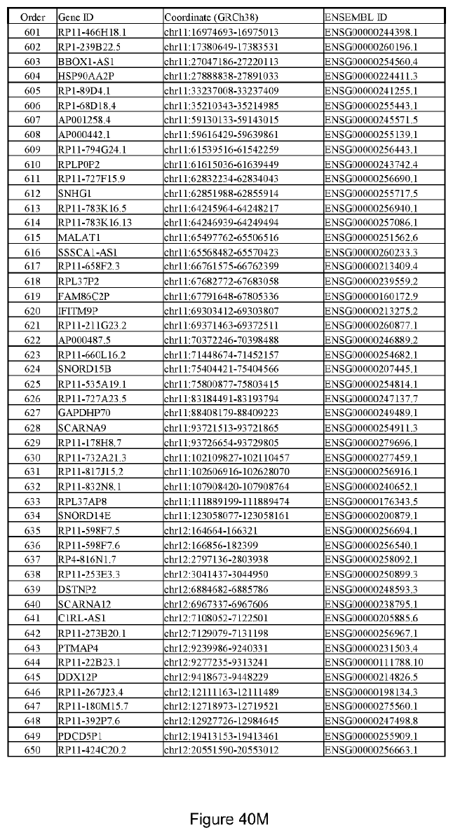

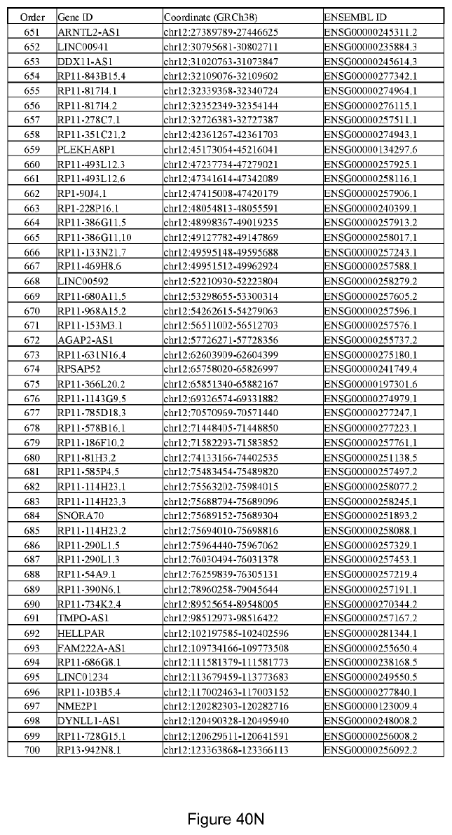

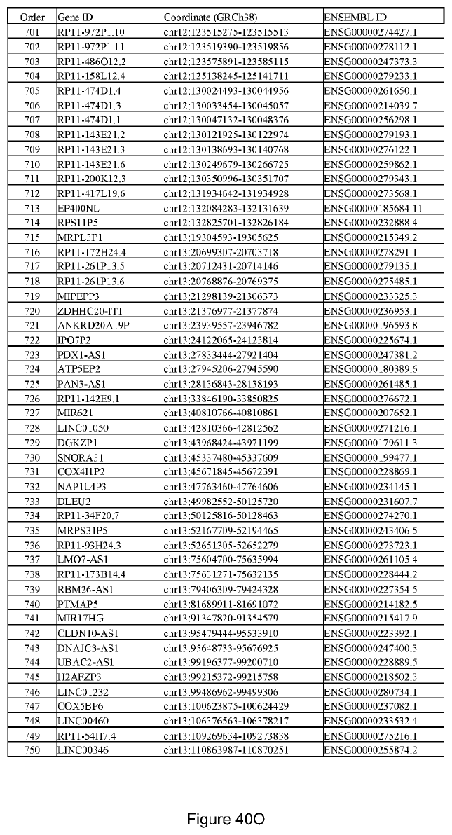

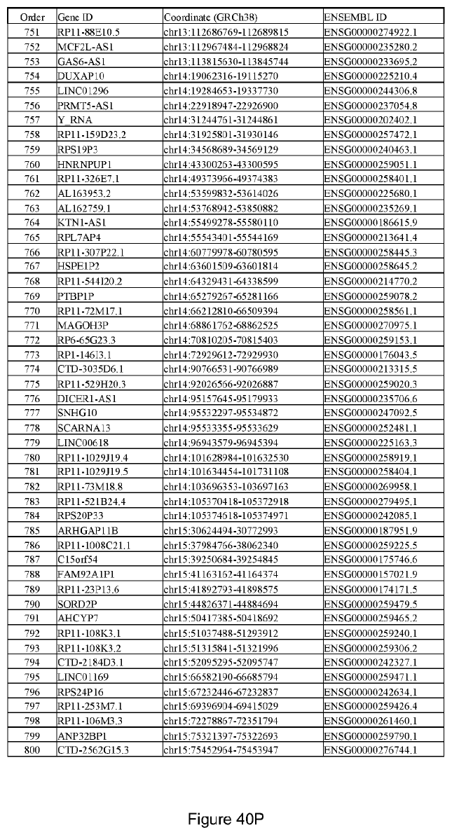

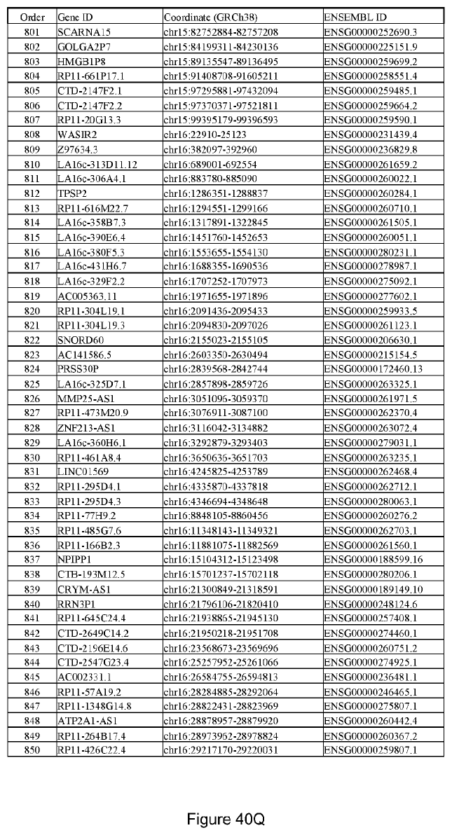

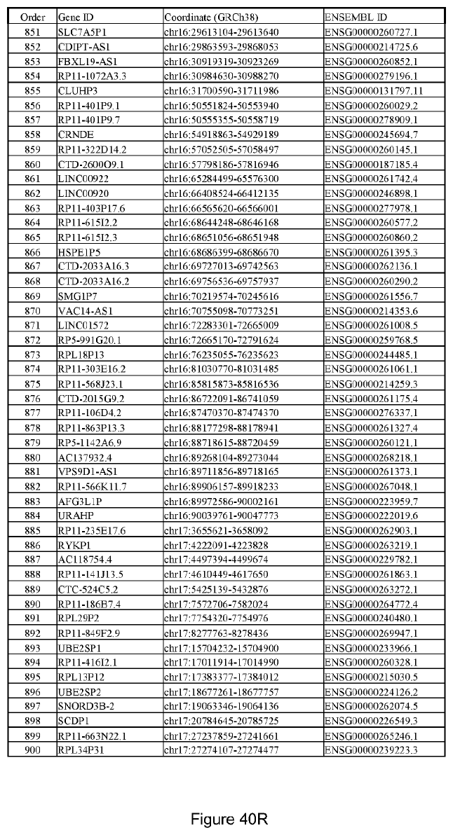

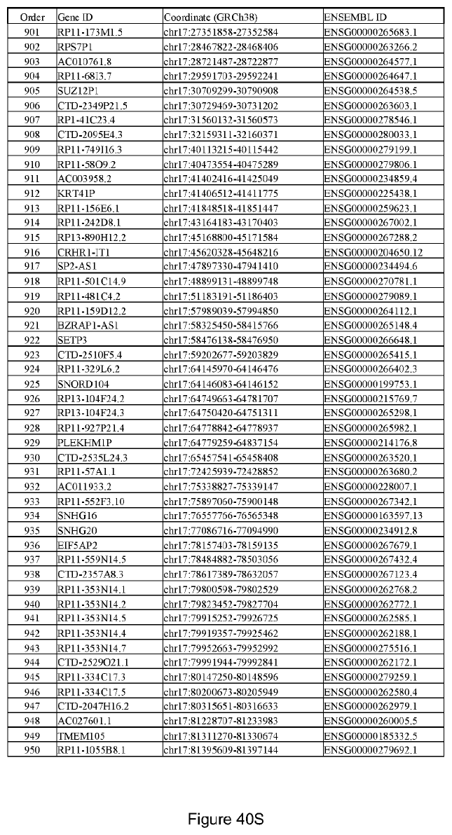

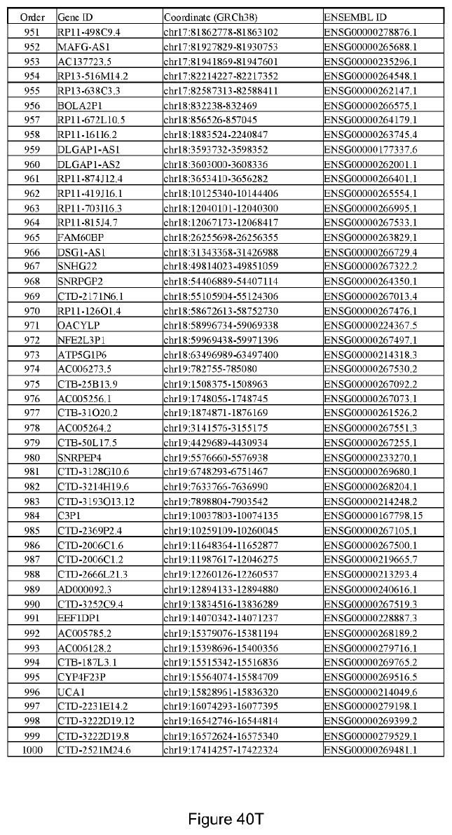

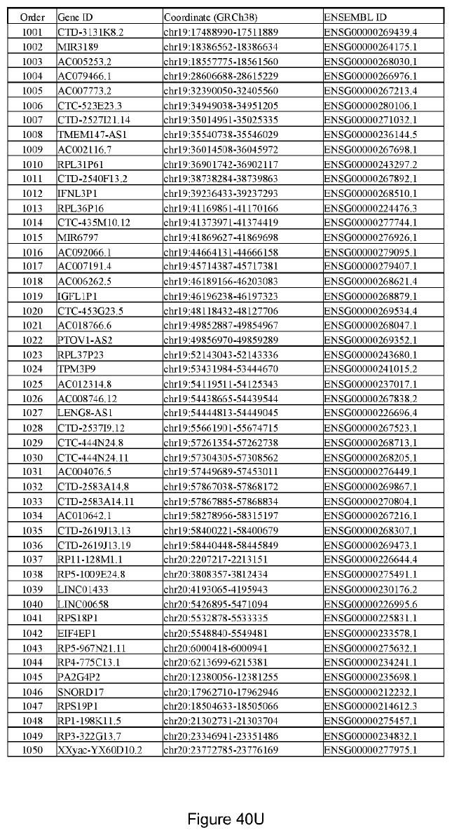

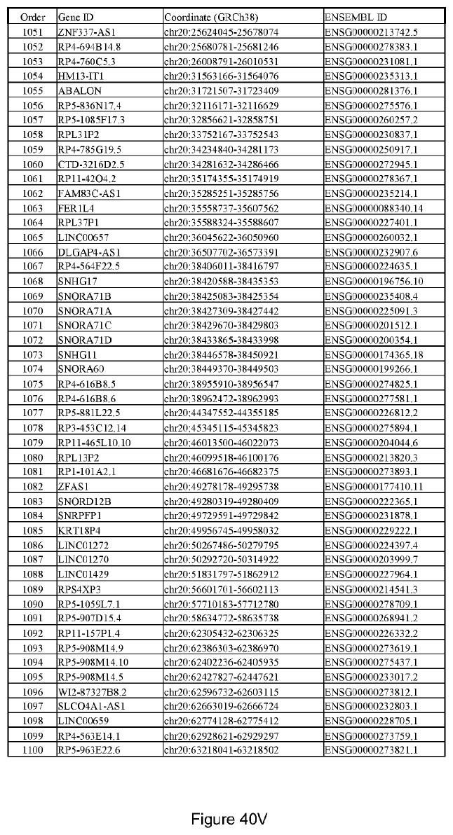

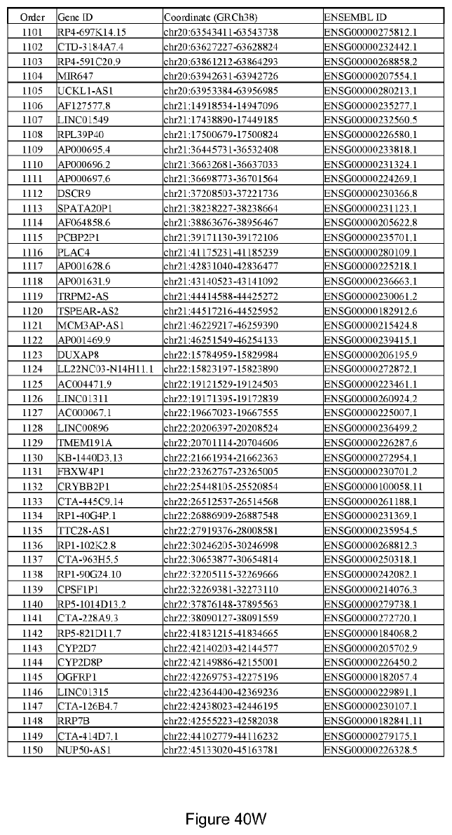

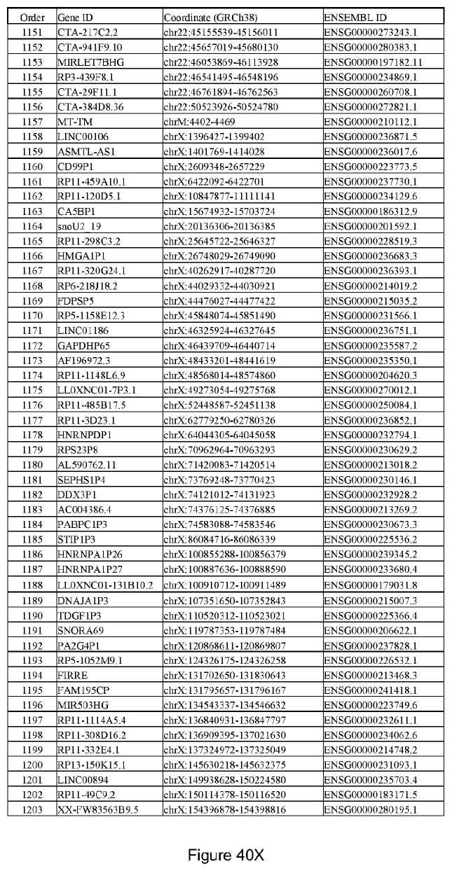

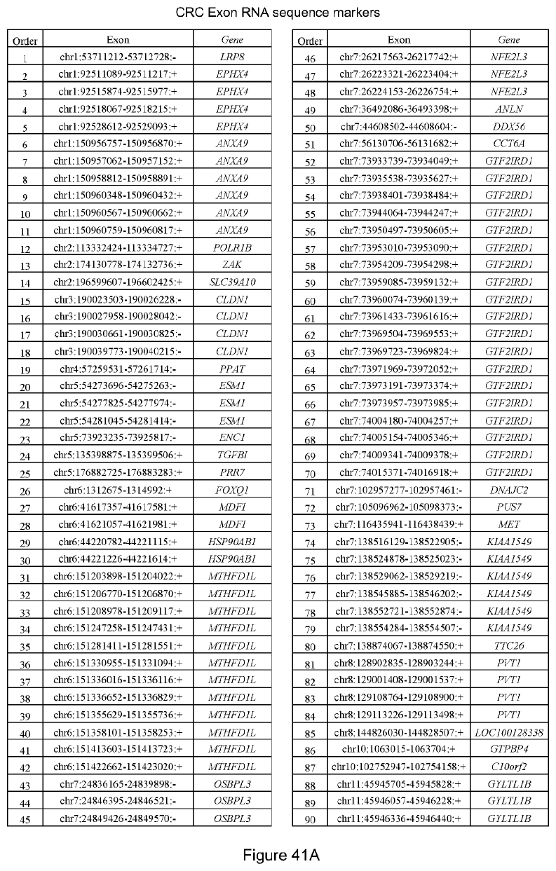

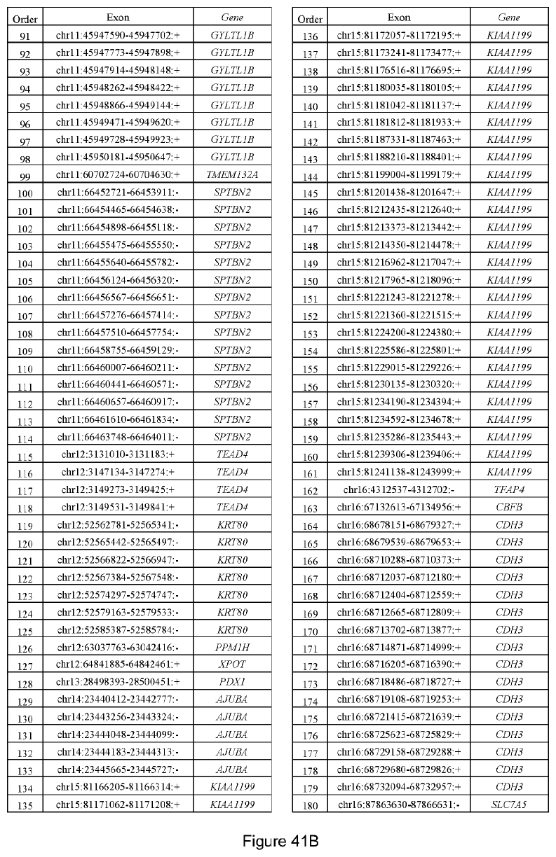

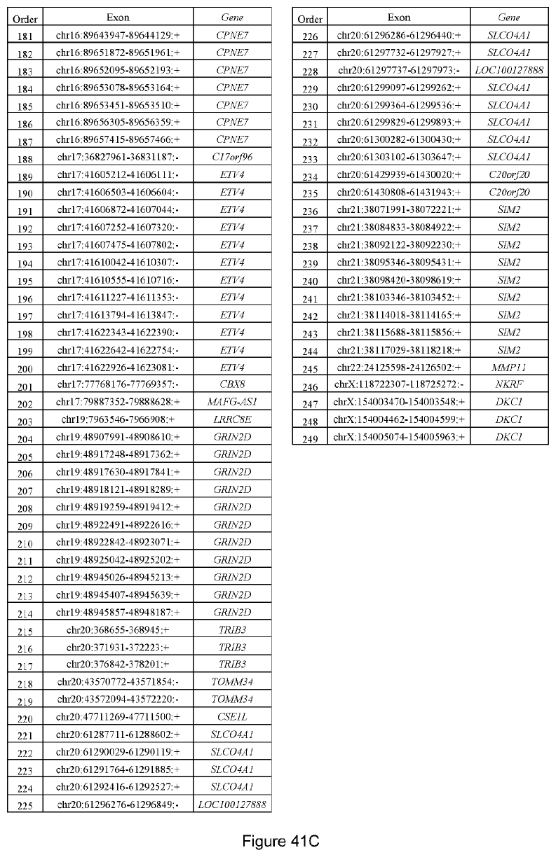

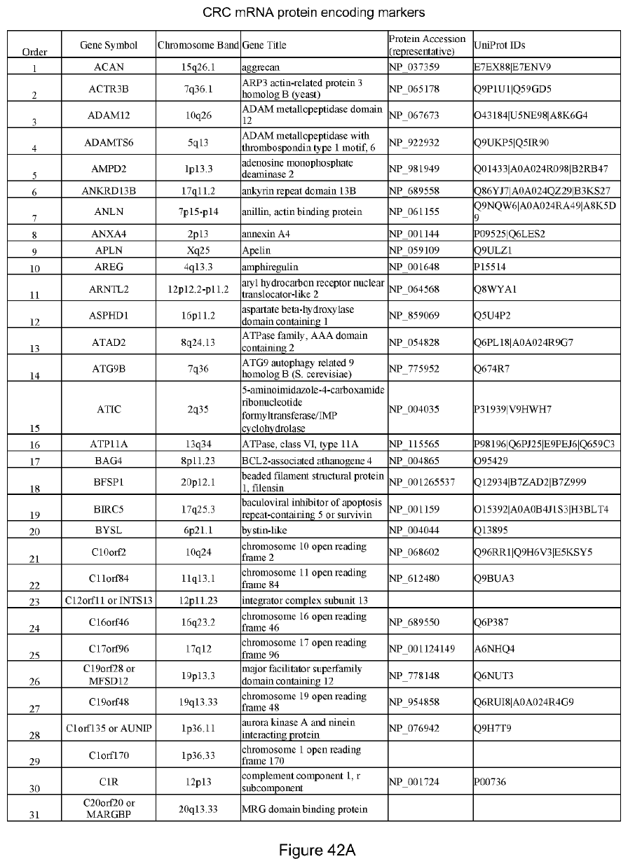

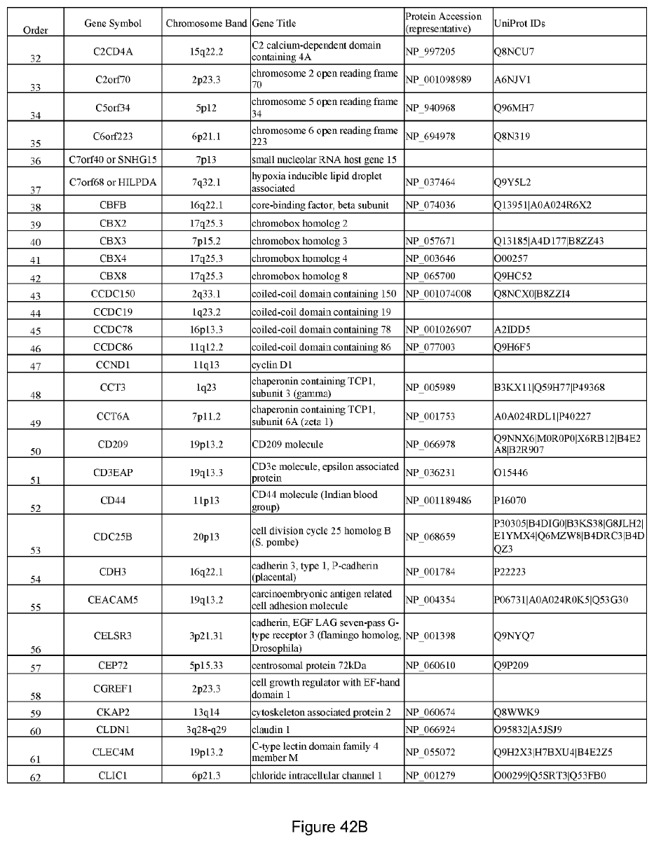

Several methods have been reported for detecting cancer-specific ncRNA or lncRNAs in the serum, plasma, or exosomes of patients with colorectal (and other) cancers; these ncRNAs include but are not limited to: NEAT_v1; NEAT_v2; CCAT1; HOTAIR; CRNDE-h; H19; MALAT1; 91H; GAS5 (Wu et al., “Nuclear-enriched Abundant Transcript 1 as a Diagnostic and Prognostic Biomarker in Colorectal Cancer,” Mol. Cancer 14:191 (2015); Zhao et al., “Combined Identification of Long Non-Coding RNA CCAT1 and HOTAIR in Serum as an Effective Screening for Colorectal Carcinoma,” Int. J. Clin. Exp. Pathol. 8(11):14131-40 (2015); Liu et al., “Exosomal Long Noncoding RNA CRNDE-h as a Novel Serum-Based Biomarker for Diagnosis and Prognosis of Colorectal Cancer,” Oncotarget 7(51):85551-85563 (2016); Slaby O, “Non-coding RNAs as Biomarkers for Colorectal Cancer Screening and Early Detection,” Adv Exp Med Biol. 937:153-70 (2016); Gao et al., “Exosomal lncRNA 91H is Associated With Poor Development in Colorectal Cancer by Modifying HNRNPK Expression,” Cancer Cell Int. 23; 18.11 (2018); Liu et al., “Prognostic and Predictive Value of Long Non-Coding RNA GAS5 and MicroRNA-221 in Colorectal Cancer and Their Effects on Colorectal Cancer Cell Proliferation, Migration and Invasion,” Cancer Biomark. 22(2):283-299 (2018); U.S. Pat. No. 9,410,206 to Hoon, et al.; U.S. Pat. No. 9,921,223 to Kalluri et al., which are hereby incorporated by reference in their entirety). Additional approaches for detecting low abundance lncRNA, ncRNA, mRNA translocations, splice variants, alternative transcripts, alternative start sites, alternative coding sequences, alternative non-coding sequences, alternative splicing, exon insertions, exon deletions, and intron insertions are described in WO2016057832A2, which is hereby incorporated by reference in its entirety, or using other suitable means known in the art. FIG. 40 provides a list of blood-based, colon cancer-specific ncRNA and lncRNA markers derived through analysis of various publicly available Affymetrix Exon ST CEL data, which were aligned to GENCODE annotations to generate ncRNA and lncRNA transriptome datasets. Comparative analyses across these datasets (various cancer types, along with normal tissues, and peripheral blood) were conducted to generate the ncRNA and lncRNA markers list (FIG. 40). Such lncRNA and ncRNA may be enriched in exosomes or other protected states in the blood. In addition, FIG. 41 provides a list of blood-based colon cancer-specific exon transcripts that may be enriched in exosomes, tumor-associated vesicles, or other protected states in the blood.

The most common protein marker for colorectal cancer is based on detecting hemoglobin from blood in the stool and is known as the FOBT or FIT test. Sensitivity and specificities (Sens.: Spec.) for these tests have been reported as: OC-Light iFOB Test (also called OC Light S FIT), manufactured by Polymedco (78.6%-97.0%:88.0%-92.8%); QuickVue iFOB, manufactured by Quidel (91.9%:74.9%); Hemosure One-Step iFOB Test, manufactured by Hemosure, Inc. (54.5%:90.5%); InSure FIT, manufactured by ClinicalGenomics (75.0%:96.6%); Hemoccult-ICT, manufactured by Beckman Coulter (23.2%-81.8%:95.8%-96.9%); Cologuard—stool FIT-DNA, manufactured by Exact Sciences (92.3%; 84.4%). The large ranges and differences in sensitivities and specificities may reflect the range from early to late cancer, as well as differences in methodology, number of samples collected, and clinical study size. Cut-off values for FIT tests may range from 10 ug protein/gram stool to 300 ug protein/gram stool (See Robertson et al., “Recommendations on Fecal Immunochemical Testing to Screen for Colorectal Neoplasia: a Consensus Statement by the US Multi-Society Task Force on Colorectal Cancer,” Gastrointest. Endow. 85(1):2-21 (2017), which is hereby incorporated by reference in its entirety).

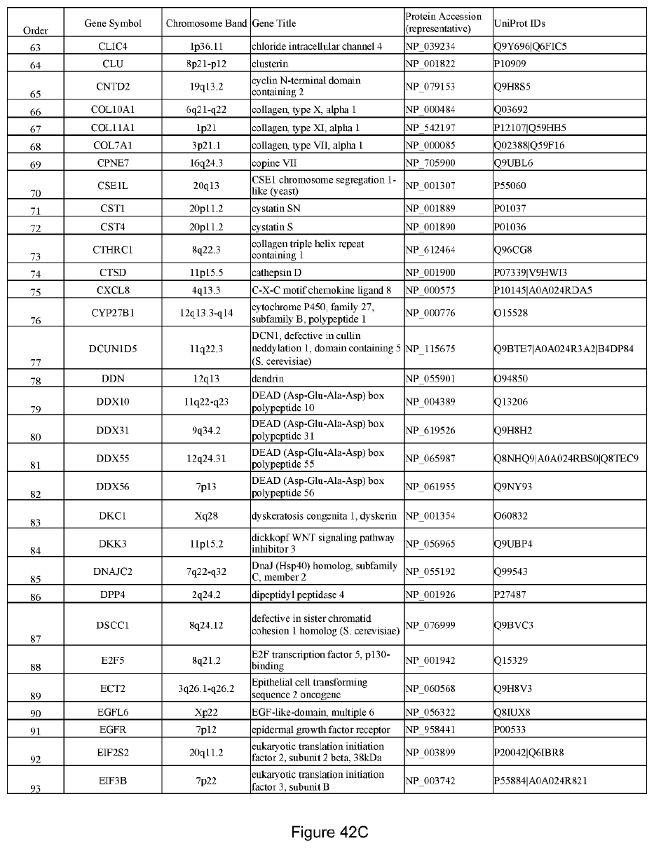

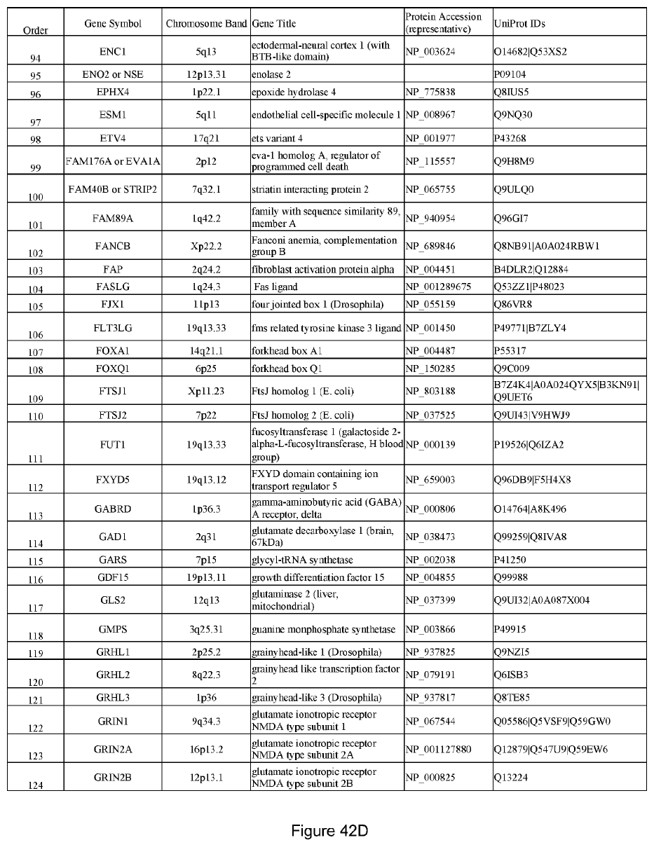

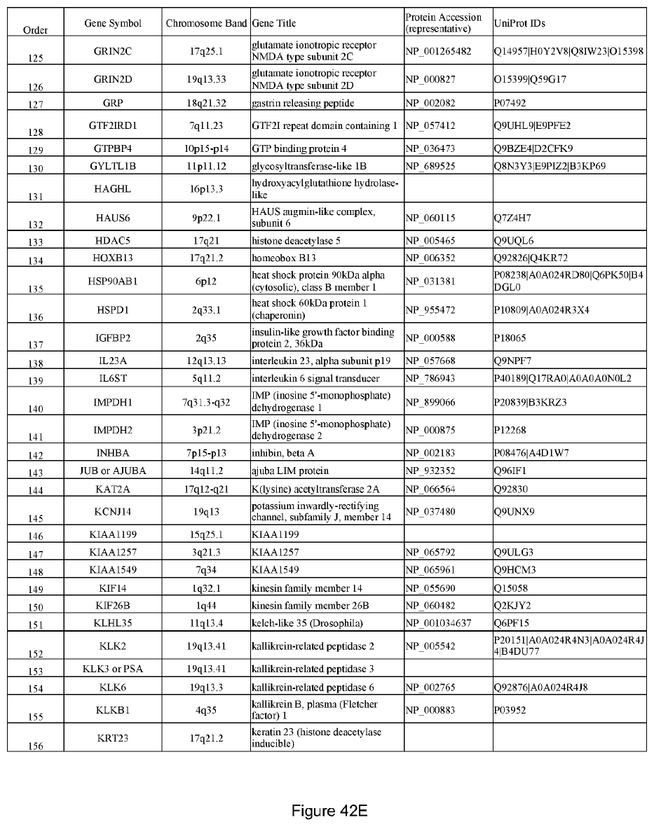

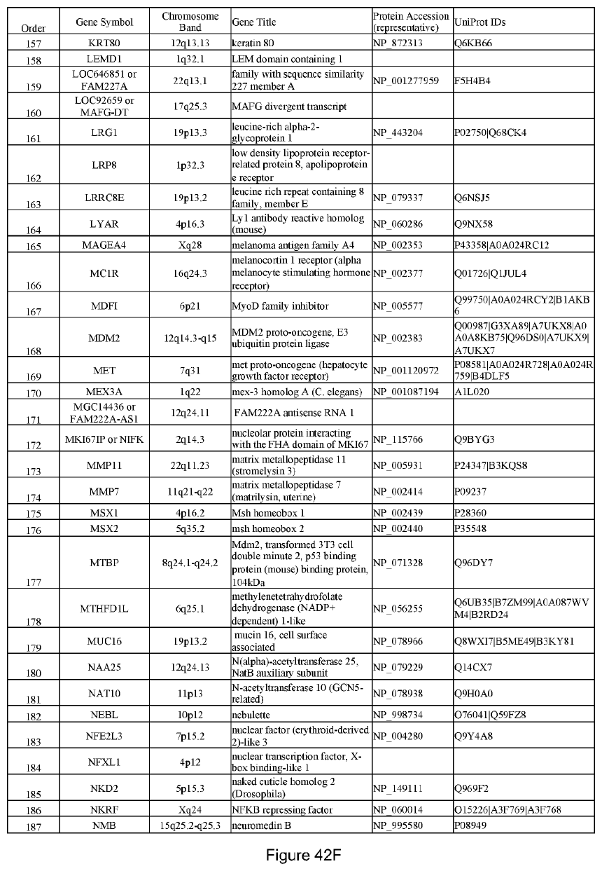

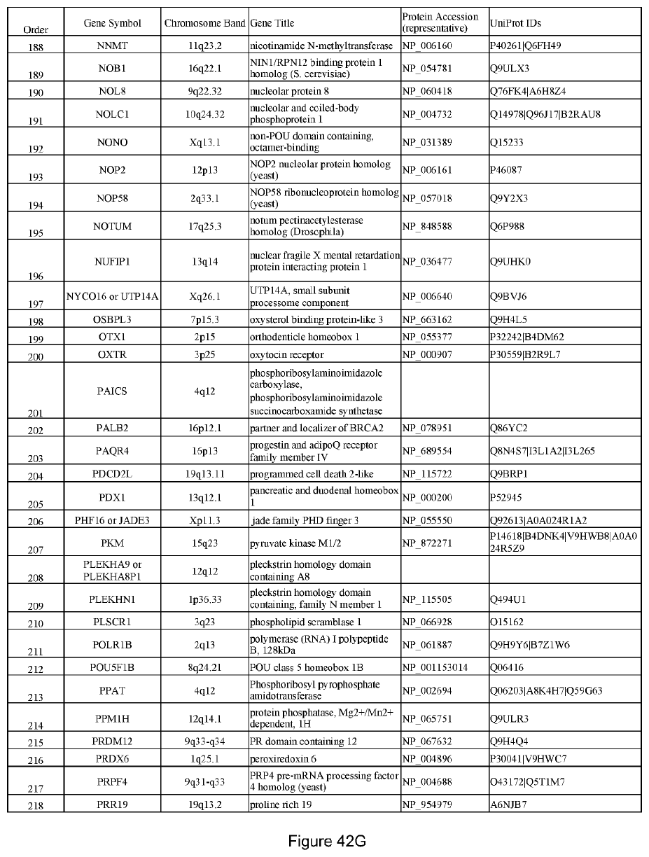

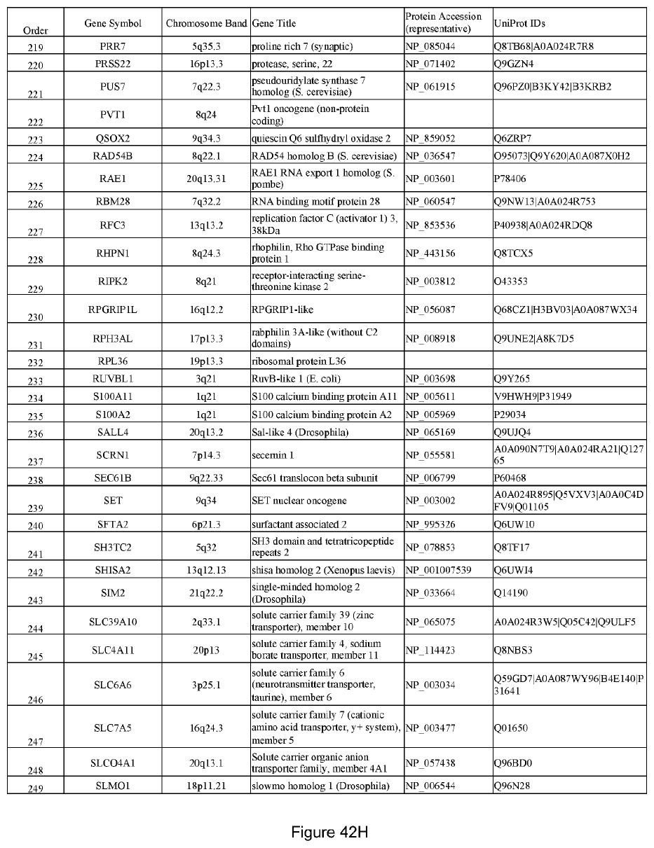

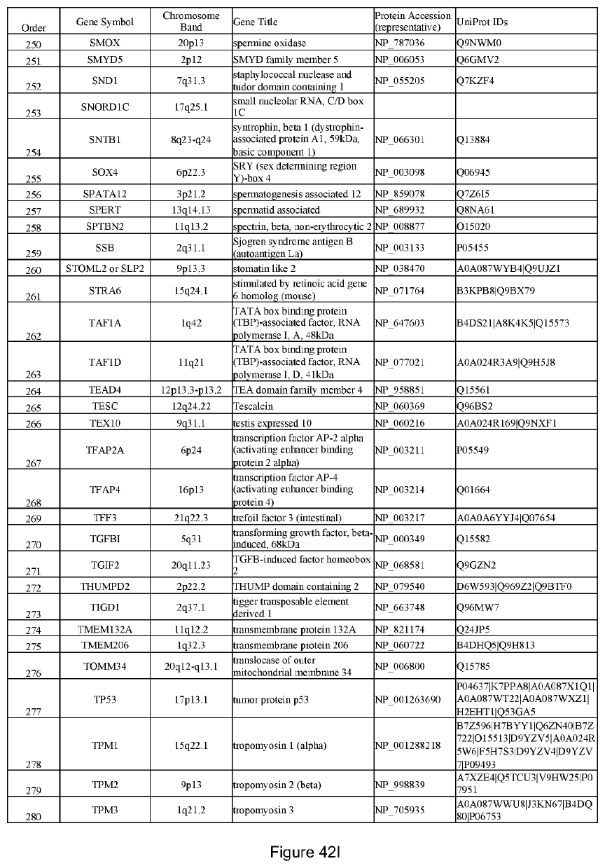

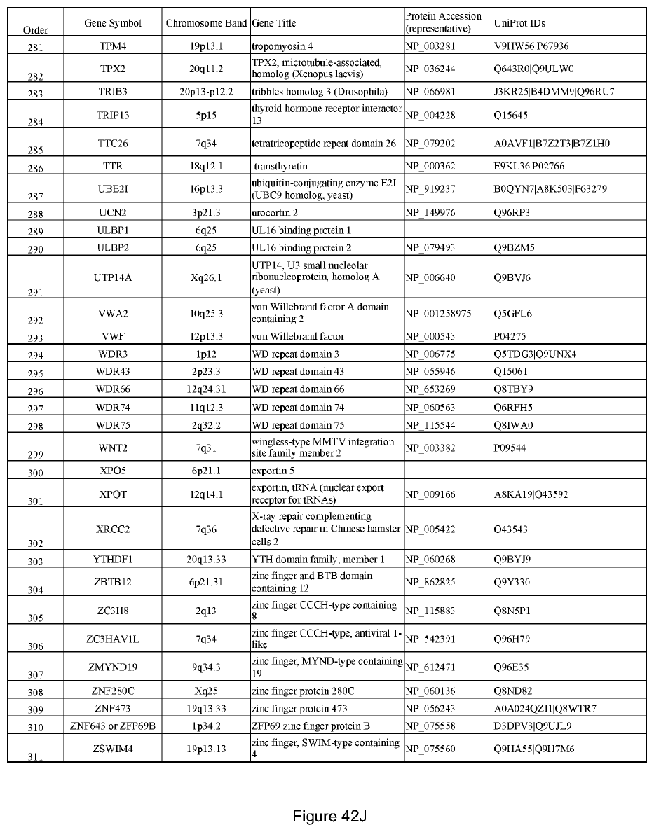

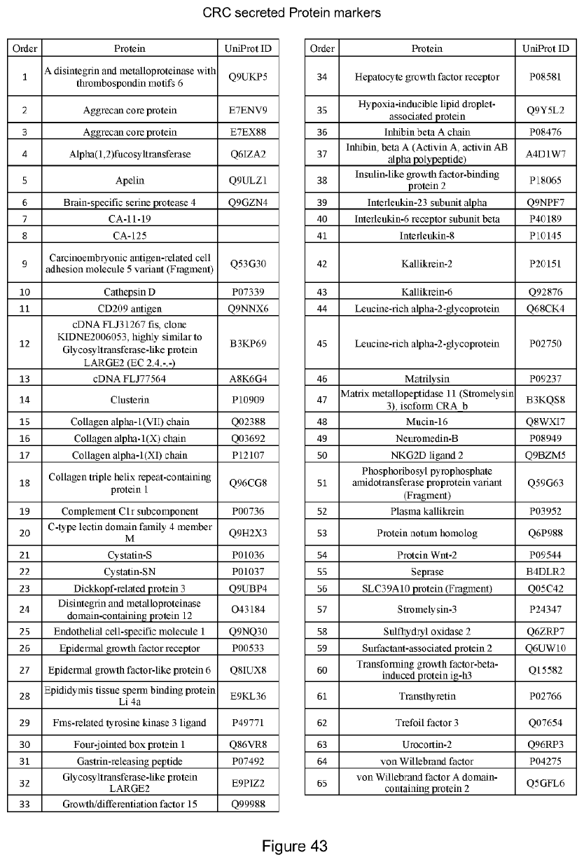

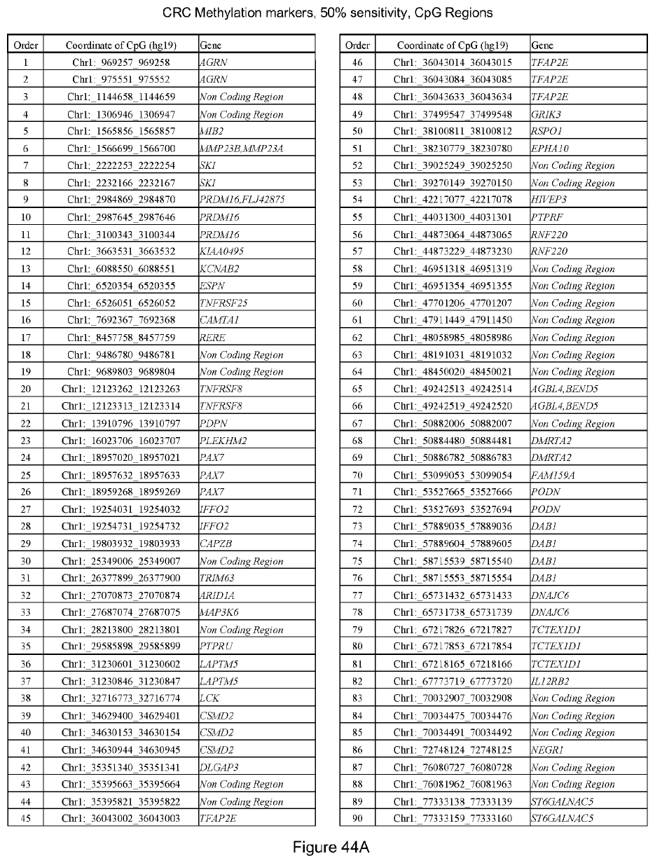

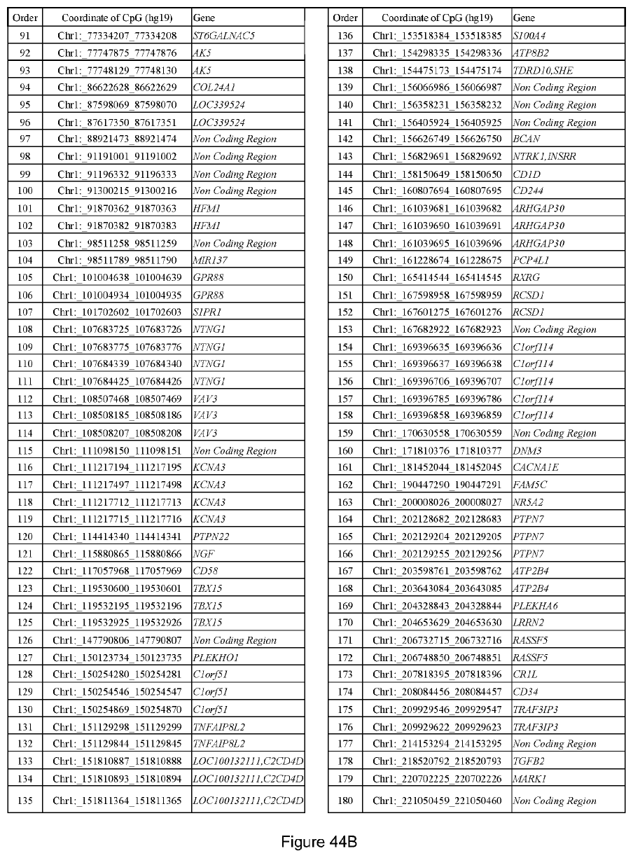

A number of tumor-associated antigens elicit an immune response within the patient, and these may be identified as autoantibodies, or indirectly as increased cytokines in the serum. Some tumor antigens may be detected directly within the serum, or on the surface of cancer-associated exosomes or extracellular vesicles, while others may be detected indirectly, for example by an increase in mRNA within cancer-associated exosomes or extracellular vesicles. These cancer-specific proteins markers may be identified through, mRNA sequences, protein expression levels, protein product concentrations, cytokines, or autoantibody to the protein product, and these markers include but are not limited to: RPH3AL; RPL36; SLP2; TP53; Survivin; ANAXA4; SEC61B; CCCAP; NYCO16; NMDAR; PLSCR1; HDAC5; MDM2; STOML2; SEC61-beta; IL8; TFF3; CA11-19; IGFBP2; DKK3; PKM2; DC-SIGN; DC-SIGNR; GDF-15; AREG; FasL; Flt3L; IMPDH2; MAGEA4; BAG4; IL6ST; VWF; EGFR; CD44; CEA; NSE; CA 19-9, CA 125; NMMT; PSA; proGRP; DPPIV/seprase complex; TFAP2A; E2F5; CLIC4; CLIC1; TPM1; TPM2; TPM3; TPM4; CTSD-30; PRDX-6; LRG1; TTR; CLU; KLKB1; C1R; KLK3; KLK2; HOXB13; GHRL2; FOXA1 (Fan et al., “Development of a Multiplexed Tumor-Associated Autoantibody-Based Blood Test for the Detection of Colorectal Cancer,” Clin. Chim. Acta. 475:157-163 (2017); Xia et al., “Prognostic Value, Clinicopathologic Features and Diagnostic Accuracy of Interleukin-8 in Colorectal Cancer: a Meta-Analysis,” PLoS One 10(4):e0123484 (2015); Li et al., “Serum Trefoil Factor 3 as a Protein Biomarker for the Diagnosis of Colorectal Cancer,” Technol. Cancer. Res. Treat. 16(4):440-445 (2017); Overholt et al., “CA11-19: a Tumor Marker for the Detection of Colorectal Cancer,” Gastrointest. Endosc. 83(3):545-551 (2016); Fung et al., “Blood-based Protein Biomarker Panel for the Detection of Colorectal Cancer,” PLoS One 10(3): e0120425 (2015); Jiang et al., “The Clinical Significance of DC-SIGN and DC-SIGNR, Which are Novel Markers Expressed in Human Colon Cancer,” PLoS One 9(12):e11474 (2014).; Chen et al., “Development and Validation of a Panel of Five Proteins as Blood Biomarkers for Early Detection of Colorectal Cancer,” Clin. Epidemiol. 9:517-526 (2017); Chen et al., “Prospective Evaluation of 64 Serum Autoantibodies as Biomarkers for Early Detection of Colorectal Cancer in a True Screening Setting,” Oncotarget 7(13):16420-32 (2016); Rho et al., “Protein and Glycomic Plasma Markers for Early Detection of Adenoma and Colon Cancer,” Gut 67(3):473-484 (2018); U.S. Pat. No. 9,518,990 to Wild et al.; U.S. Pat. No. 9,835,636 to Chan et al.; U.S. Pat. No. 9,885,718 to Man et al.; U.S. Pat. No. 9,889,135 to Andy Koff et al.; U.S. Pat. No. 9,903,870 to Speicher et al.; U.S. Pat. No. 9,914,974 to Bajic et al.; U.S. Pat. No. 9,983,208 to Choi et al; U.S. Pat. No. 10,030,271 to Scher et al., which are hereby incorporated by reference in their entirety). Additional approaches for detecting low abundance mRNA translocations, splice variants, alternative transcripts, alternative start sites, alternative coding sequences, alternative non-coding sequences, alternative splicing, exon insertions, exon deletions, and intron insertions are described in WO2016057832A2, which is hereby incorporated by reference in its entirety, or using other suitable means known in the art, FIG. 42 provides a list of cancer protein markers, identified through mRNA sequences, protein expression levels, protein product concentrations, cytokines, or autoantibody to the protein product arising from Colorectal tumors, which may be identified in the blood, either within exosomes, other protected states, tumor-associated vesicles, or free within the plasma. FIG. 43 provides protein markers that can be secreted by Colorectal tumors into the blood. A comparative analysis was performed across various TCGA datasets (tumors, normals), followed by an additional bioinformatics filter (Meinken et al., “Computational Prediction of Protein Subcellular Locations in Eukaryotes: an Experience Report,” Computational Molecular Biology 2(1):1-7 (2012), which is hereby incorporated by reference in its entirety), which predicts the likelihood that the translated protein is secreted by the cells.

The distribution of mutations in colorectal cancers are available in the public COSMIC database, with the 20 most commonly altered genes listed as: APC; TP53; KRAS; FAT4; LRP1B; PIK3CA; TGFBR2; ACVR2A; BRAF; ZFHX3; KMT2C; KMT2D; FBXW7; SMAD4; ARID1A; TRRAP; RNF43: FAT1; TCF7L2; PREX2 (Forbes et al., “COSMIC: Exploring the World's Knowledge of Somatic Mutations in Human Cancer,” Nucleic Acids Res. 43(Database issue):D805-811 (2015), which is hereby incorporated by reference in its entirety). Analysis of TCGA COADREAD mutational dataset, however indicate the following genes have at least 10% mutation rate among colorectal cancer primary tumors: APC, TP53, KRAS, TTN, SYNE1, PIK3CA, FAT4, MUC16, FBXW7, LRP1B, LRP2, DNAH5, DMD, ANK2, RYR2, FLG, HMCN1, FAT2, TCF7L2, CSMD3, USH2A, SDK1, CSMD1, COL6A3, DNAH2, SMAD4, PKHD1, FAM123B, ATM, ACVR2A, MDN1, DCHS2, ZFHX4, CUBN, CSMD2, FREM2, RYR1, TGFBR2, RYR3, SACS, DNAH10, ABCA12, BRAF, ODZ1, PCDH9, MACF1, AHNAK2. In addition to the approaches described herein, there are several approaches for enriching for and detecting low-abundance mutations either at the DNA or mRNA level (for example, mRNA within exosomes), including but not limited to next generation sequencing, allele-specific PCR, ARMS, primer-extension PCR, using blocking primers, full-COLD-PCR, fast-COLD-PCR, ice-COLD-PCR, E-ice-COLD-PCR, TT-COLD-PCR, etc. (Mauger et al., “COLD-PCR Technologies in the Area of Personalized Medicine: Methodology and Applications,” Mol. Diagn Ther. (3):269-283 (2017); Sefrioui et al., “Comparison of the Quantification of KRAS Mutations by Digital PCR and E-ice-COLD-PCR in Circulating-Cell-Free DNA From Metastatic Colorectal Cancer Patients,” Clin. Chim. Acta. 465:1-4 (2017); U.S. Pat. No. 9,062,350 to Platica; U.S. Pat. No. 9,598,735 to Song et al., which are hereby incorporated by reference in their entirety). Additional approaches for detecting low abundance mutations are described in WO2016057832A2, which is hereby incorporated by reference in its entirety, or using other suitable means known in the art.

The best studied blood-based methylation markers for CRC detection are located in the promoter region of the SEPT9 gene (Church et al., “Prospective Evaluation of Methylated SEPT9 in Plasma for Detection of Asymptomatic Colorectal Cancer,” Gut 63(2):317-325 (2014); Lofton-Day et al., “DNA Methylation Biomarkers for Blood-Based Colorectal Cancer Screening,” Clinical Chemistry 54(2):414-423 (2008); Potter et al., “Validation of a Real-time PCR-based Qualitative Assay for the Detection of Methylated SEPT9 DNA in Human Plasma,” Clinical Chemistry 60(9):1183-1191 (2014); Ravegnini et al., “Simultaneous Analysis of SEPT9 Promoter Methylation Status, Micronuclei Frequency, and Folate-Related Gene Polymorphisms: The Potential for a Novel Blood-Based Colorectal Cancer Biomarker,” International Journal of Molecular Sciences 16(12):28486-28497 (2015); Toth et al., “Detection of Methylated SEPT9 in Plasma is a Reliable Screening Method for Both Left- and Right-sided Colon Cancers,” PloS One 7(9):e46000 (2012); Toth et al., “Detection of Methylated Septin 9 in Tissue and Plasma of Colorectal Patients With Neoplasia and the Relationship to the Amount of Circulating Cell-free DNA,” PloS One 9(12):e115415 (2014); Warren et al., “Septin 9 Methylated DNA is a Sensitive and Specific Blood Test for Colorectal Cancer,” BMC Medicine 9:133 (2011), which are hereby incorporated by reference in their entirety), and other potential markers for CRC diagnostics include CpG sites on promoter regions of THBD, C9orf50, ZNF154, AGBL4, FLI1, and TWIST1 (Lange et al., “Genome-scale Discovery of DNA-methylation Biomarkers for Blood-based Detection of Colorectal Cancer,” PloS One 7(11):e50266 (2012); Margolin et al., “Robust Detection of DNA Hypermethylation of ZNF154 as a Pan-Cancer Locus with in Silico Modeling for Blood-Based Diagnostic Development,” The Journal of Molecular Diagnostics: JMD 18(2):283-298 (2016); Lin et al., “Clinical Relevance of Plasma DNA Methylation in Colorectal Cancer Patients Identified by Using a Genome-Wide High-Resolution Array,” Ann. Surg. Oncol. 22 Suppl 3:S1419-1427 (2015), which are hereby incorporated by reference in their entirety).

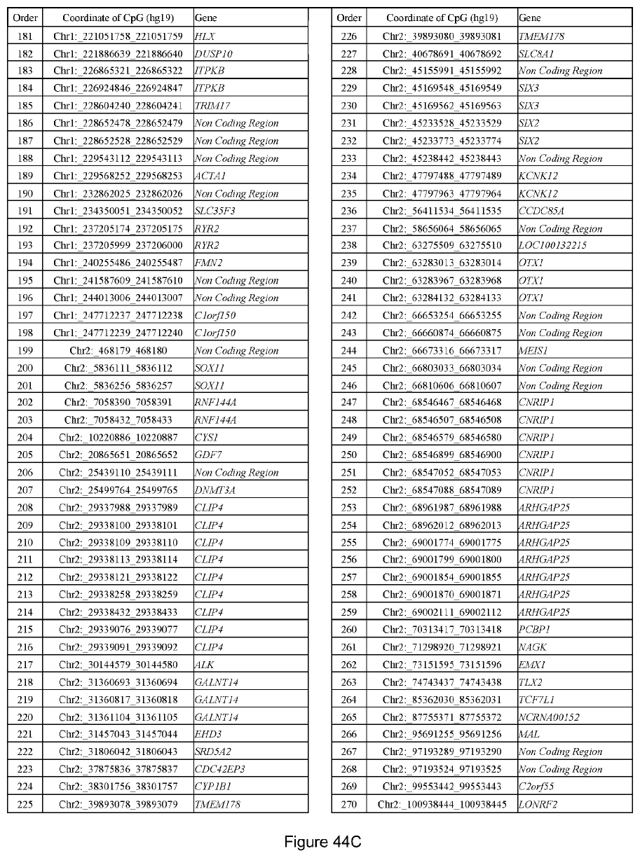

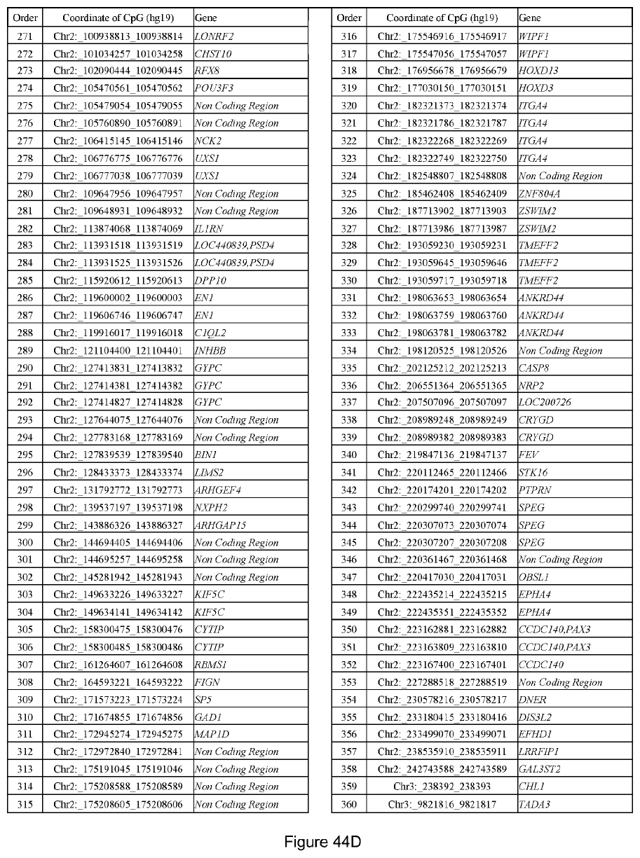

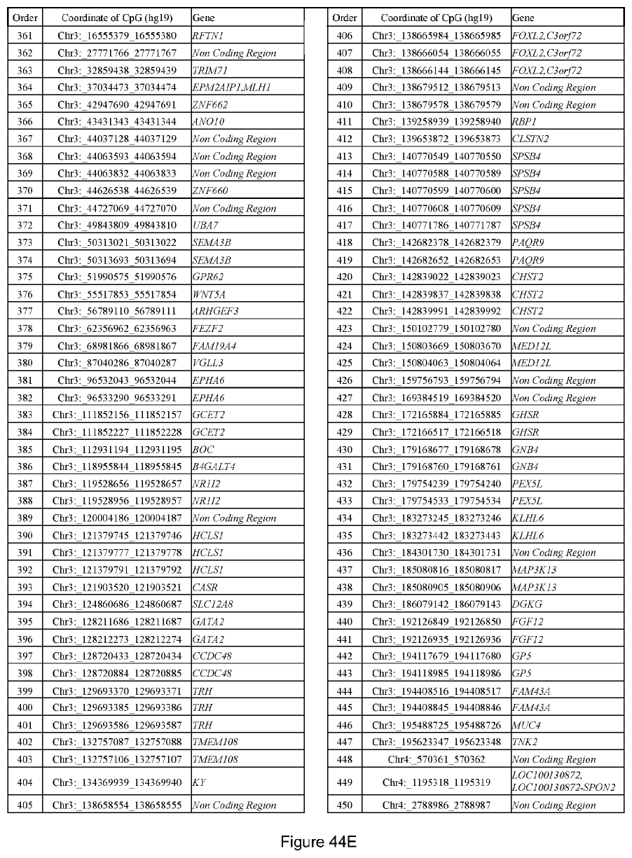

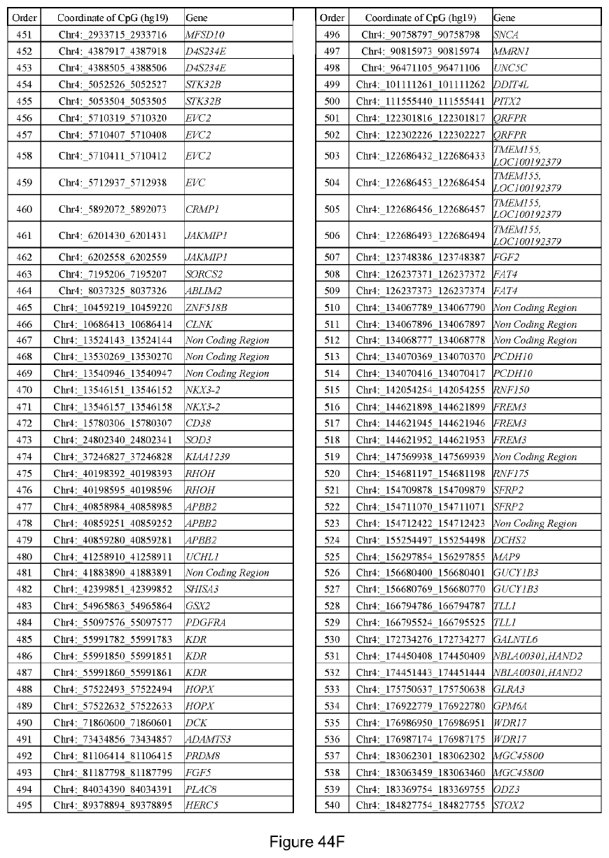

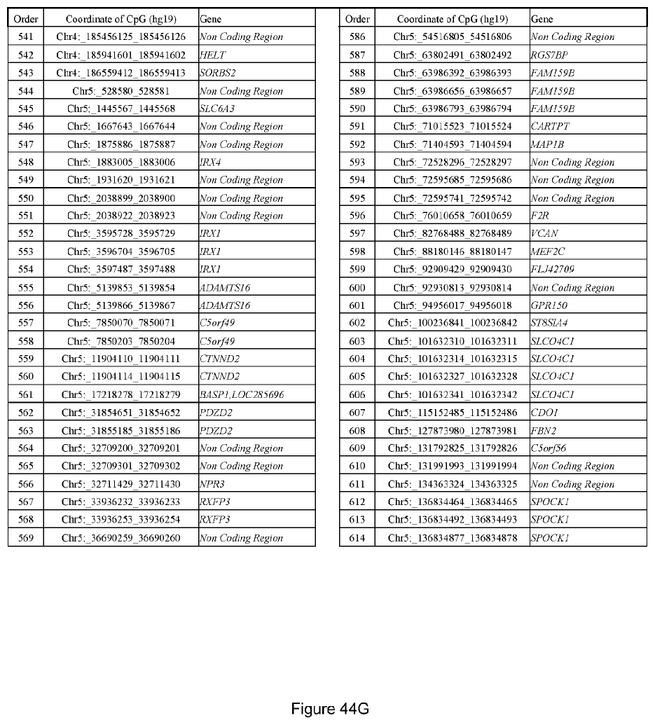

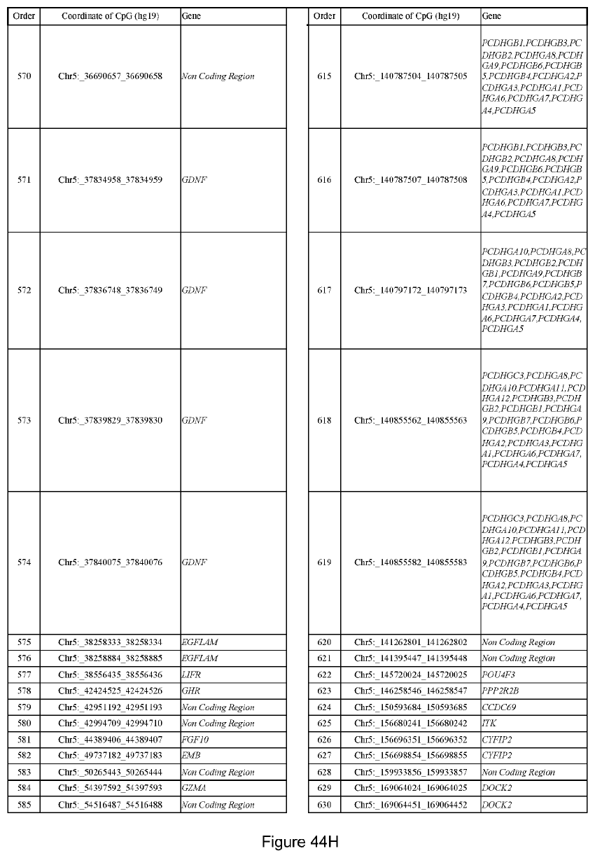

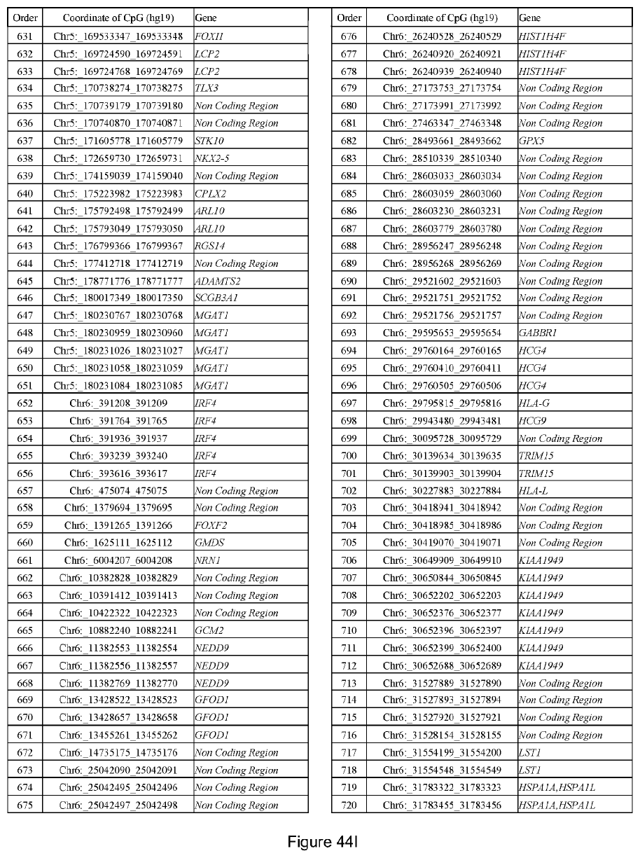

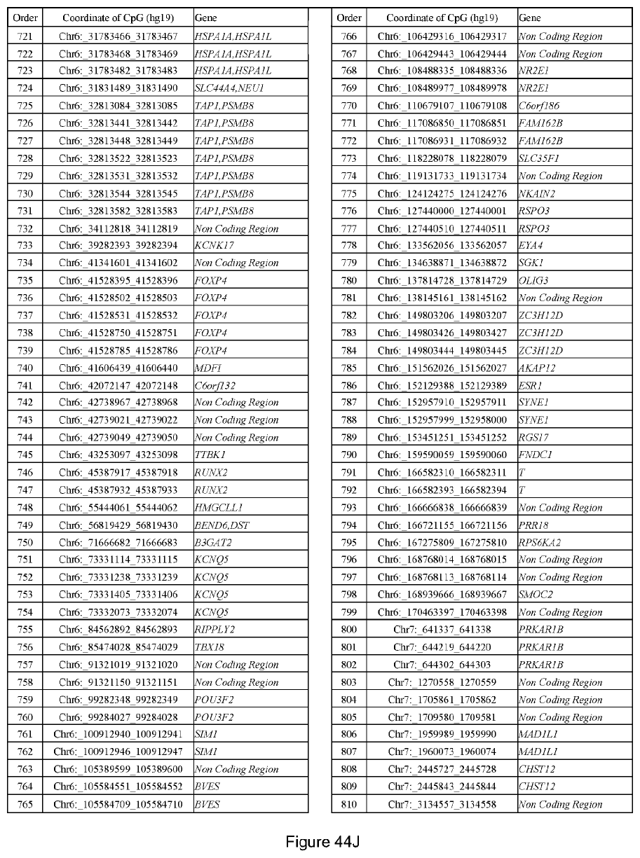

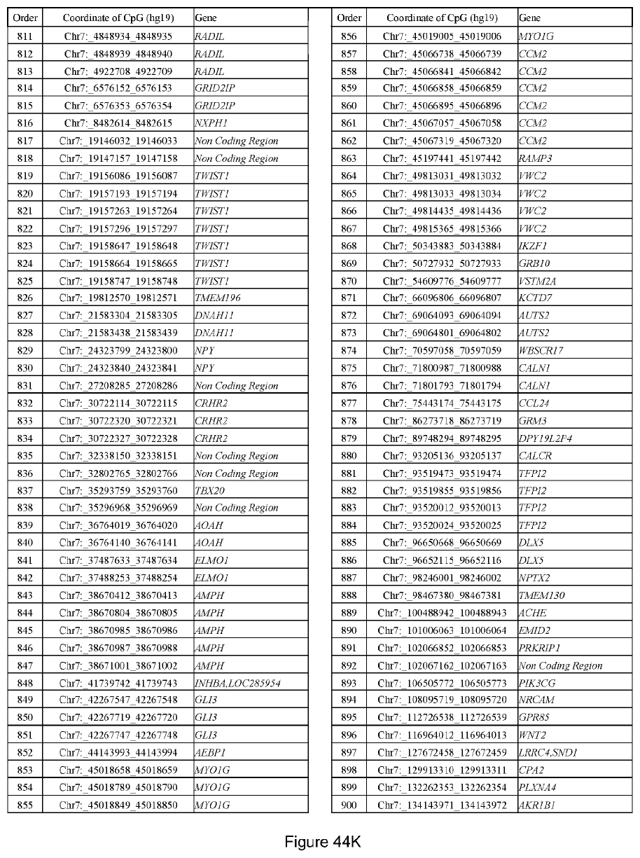

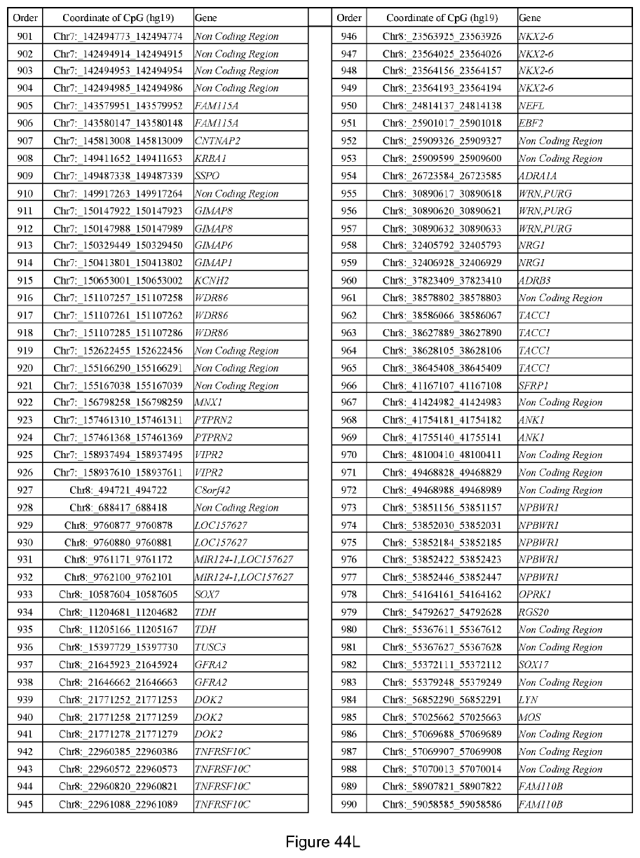

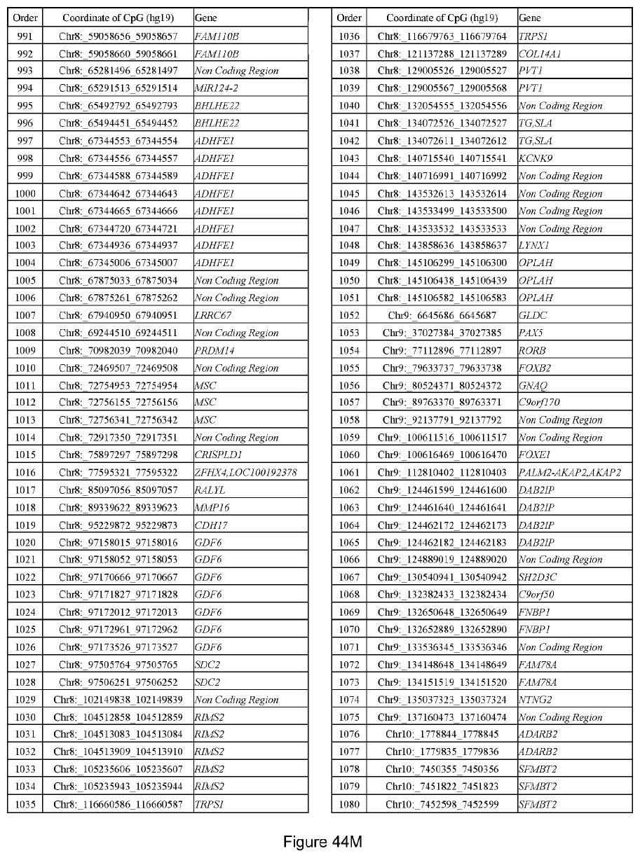

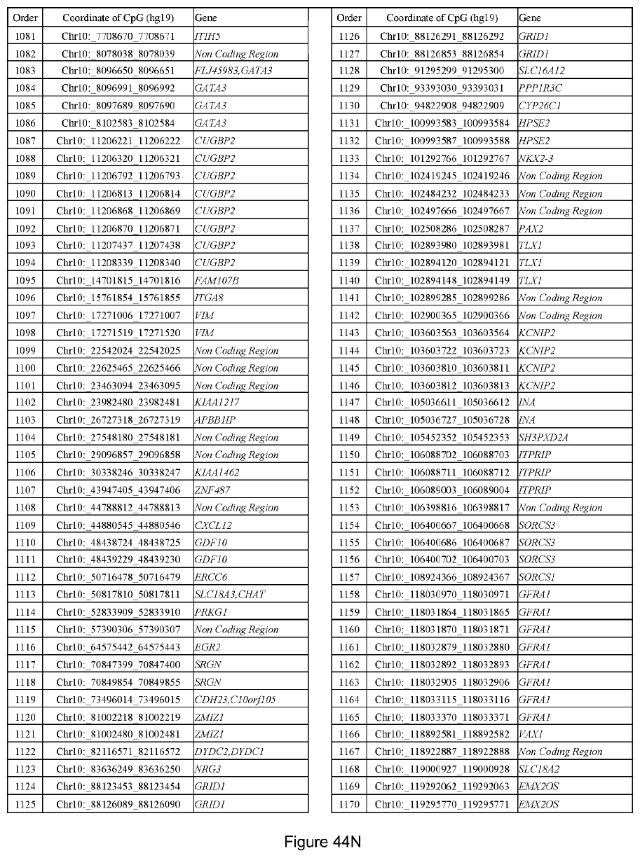

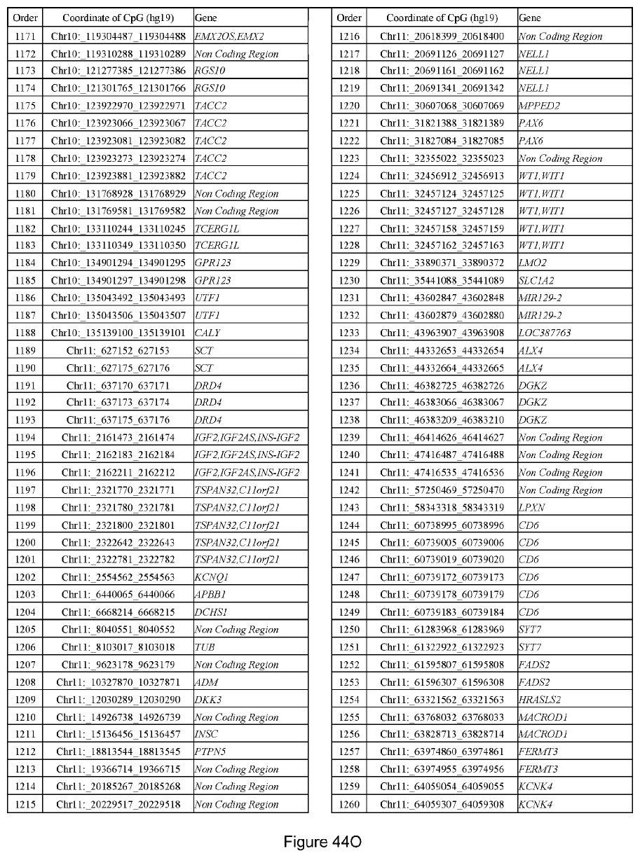

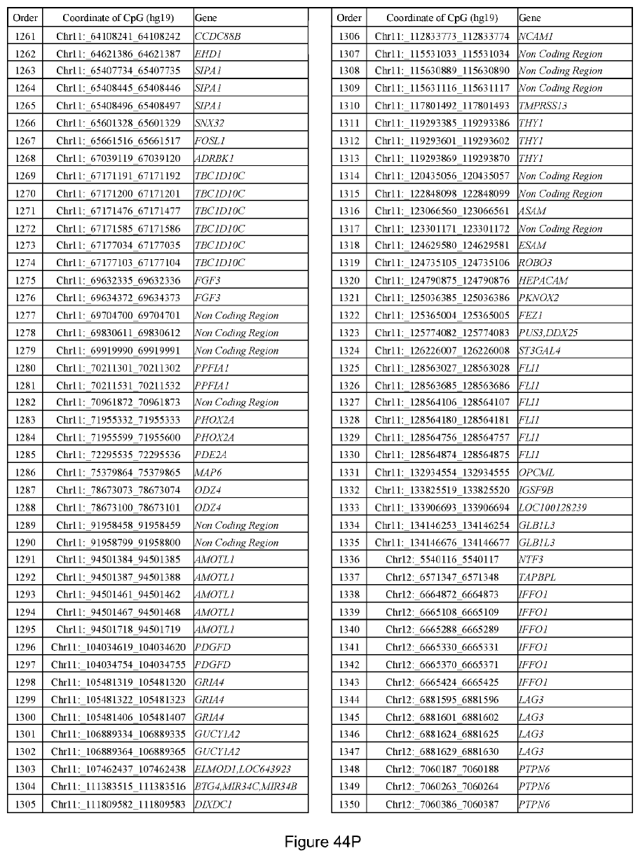

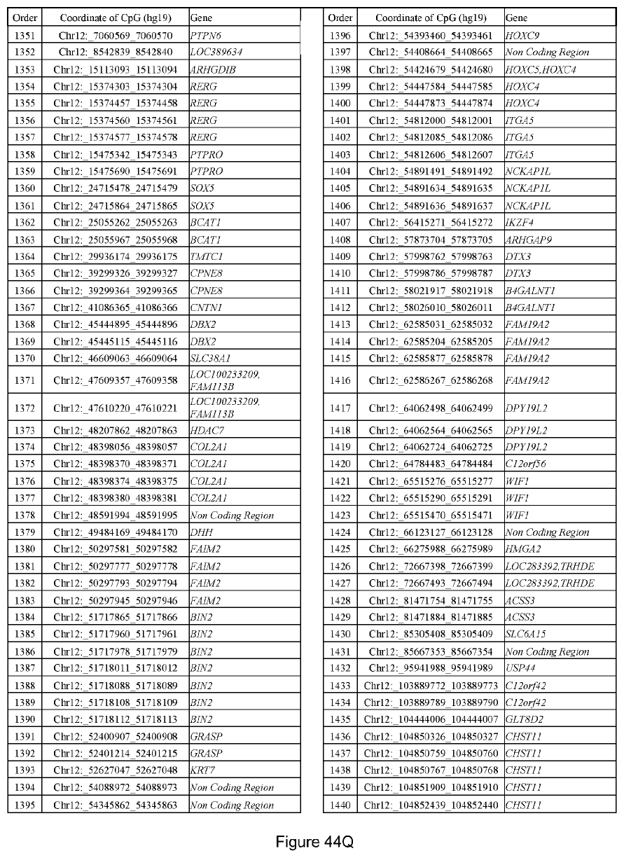

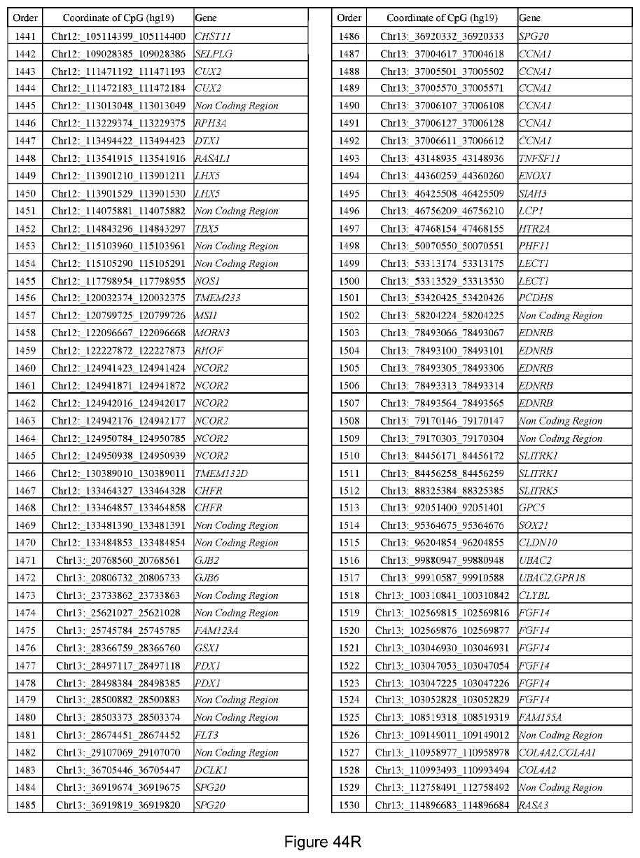

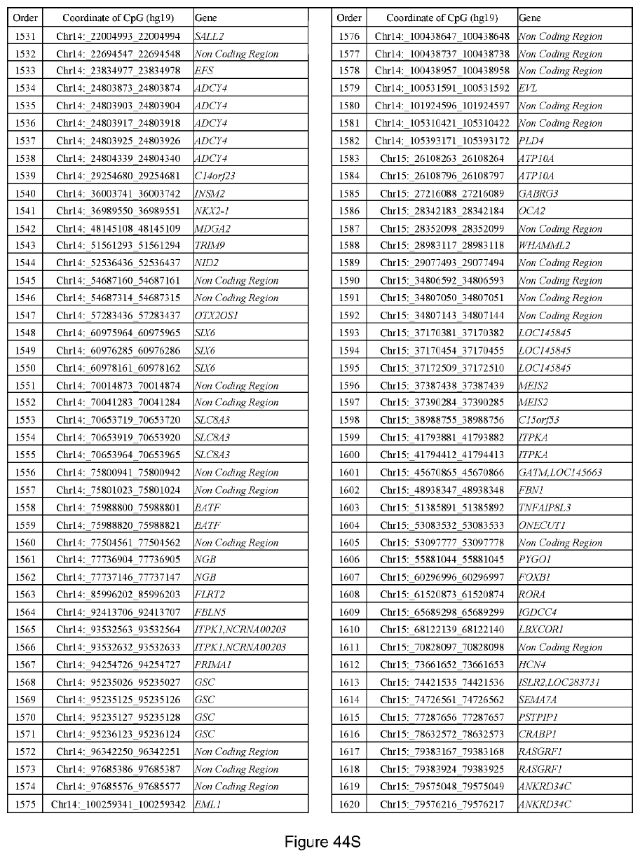

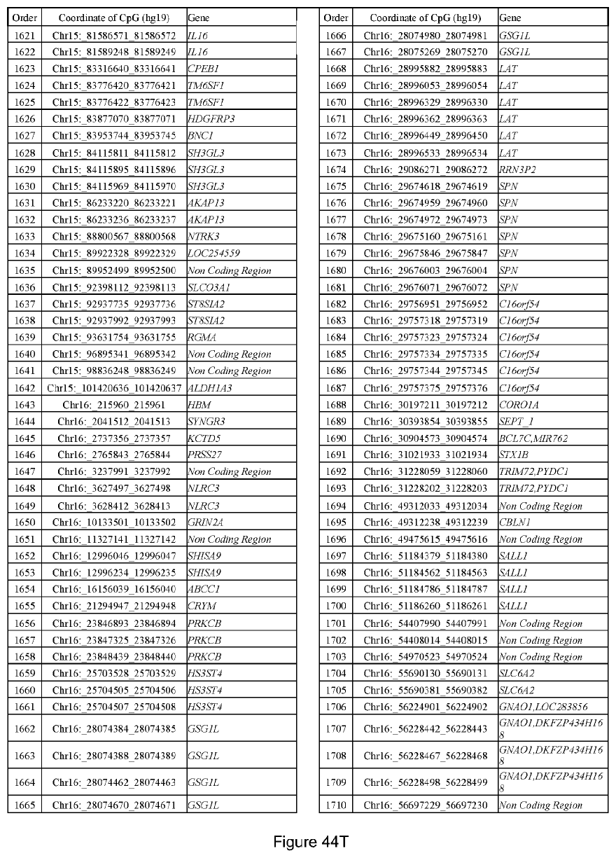

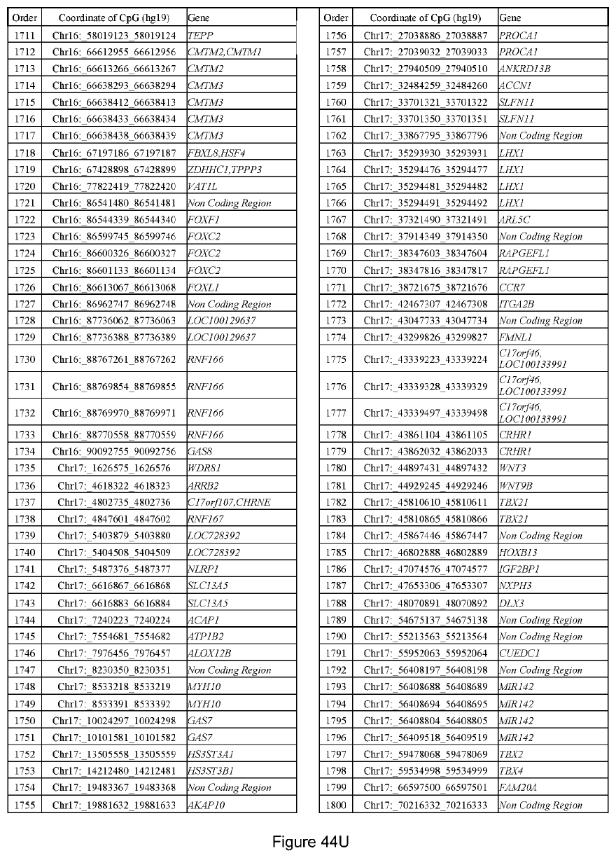

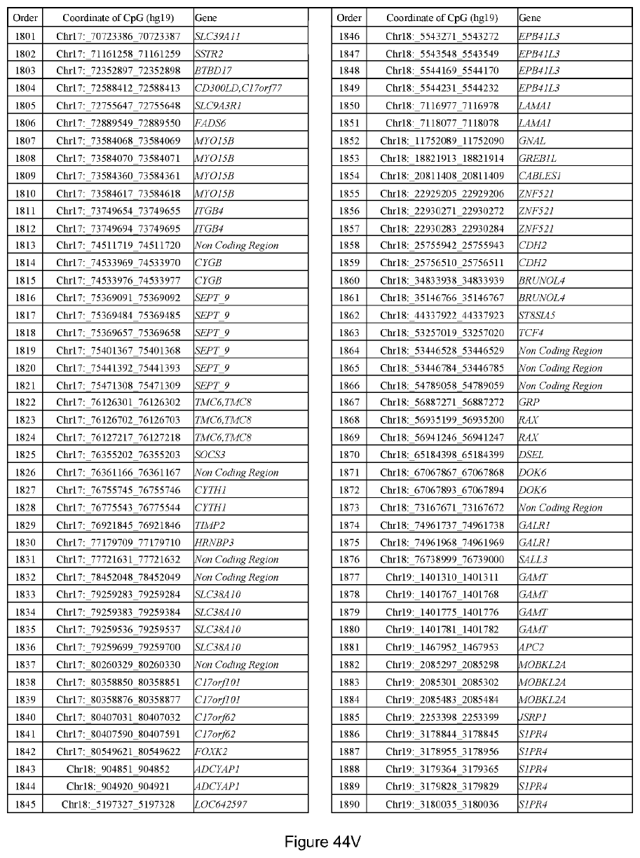

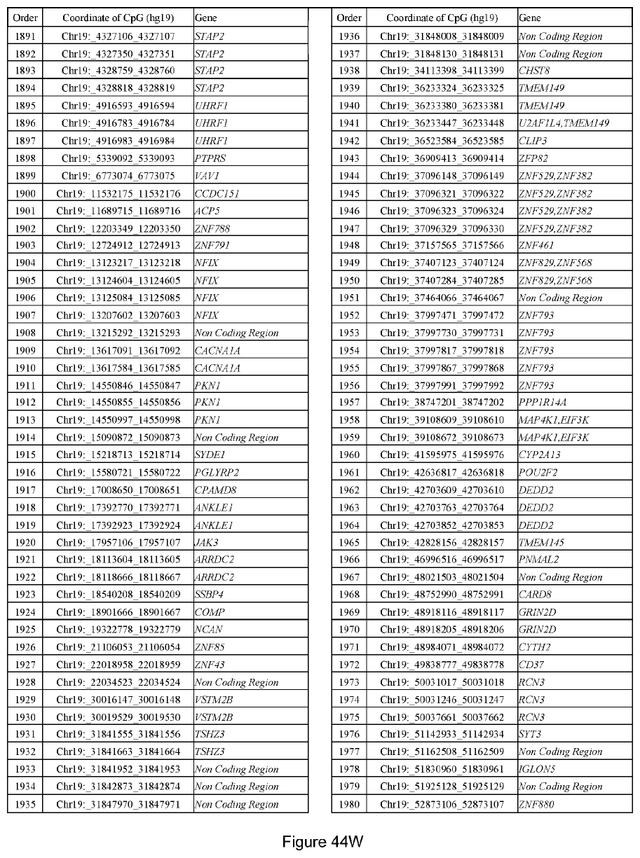

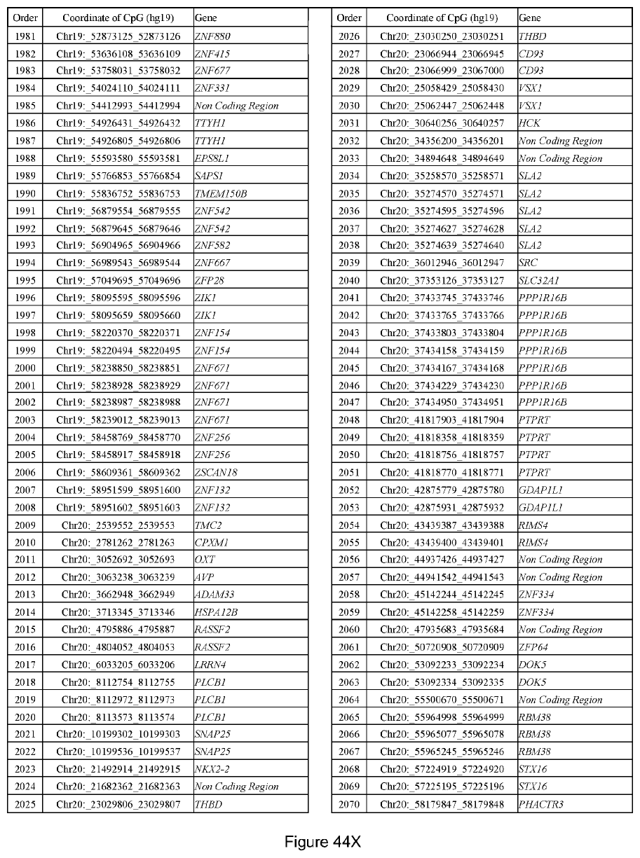

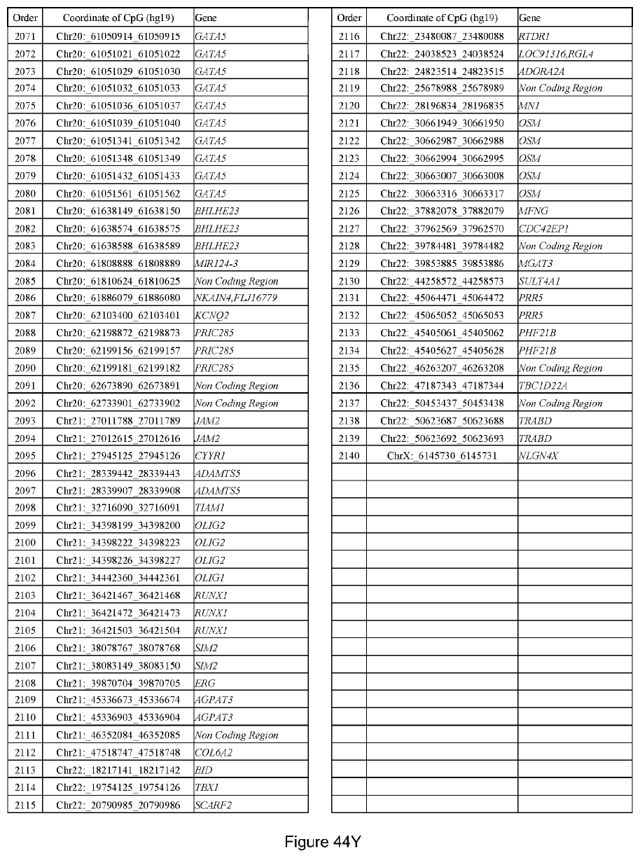

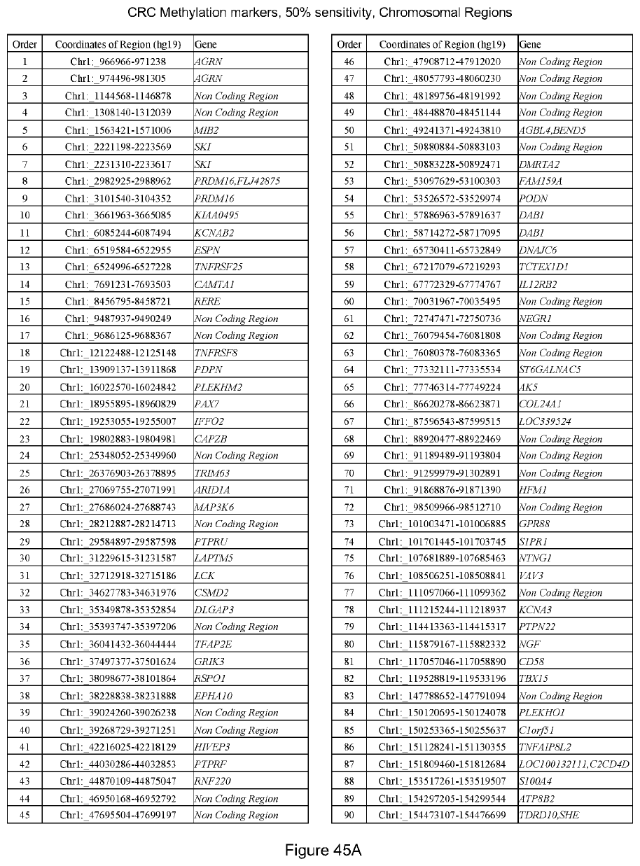

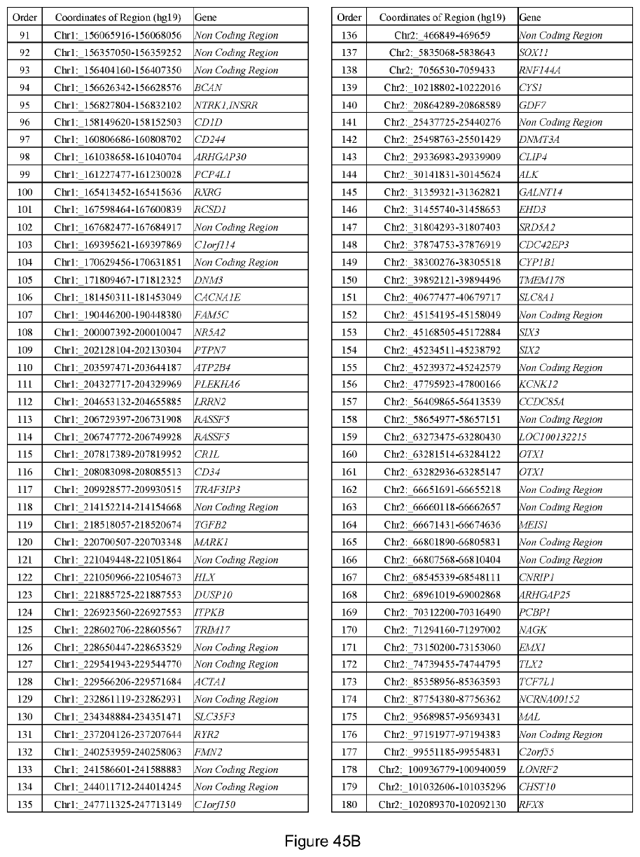

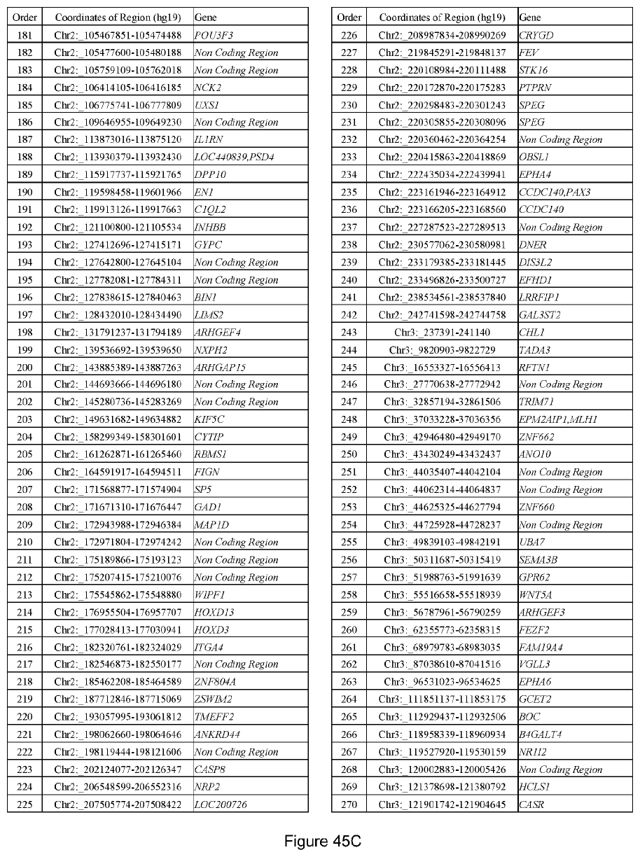

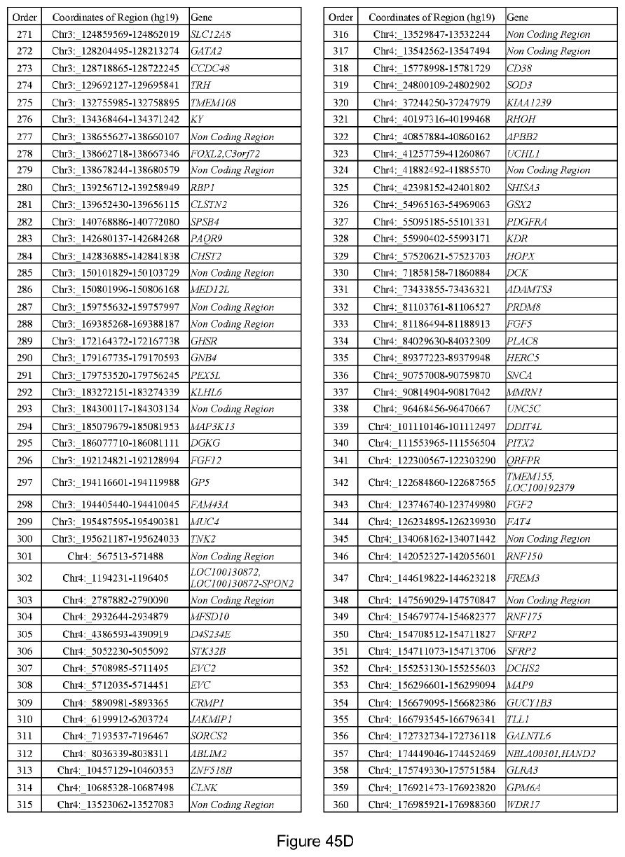

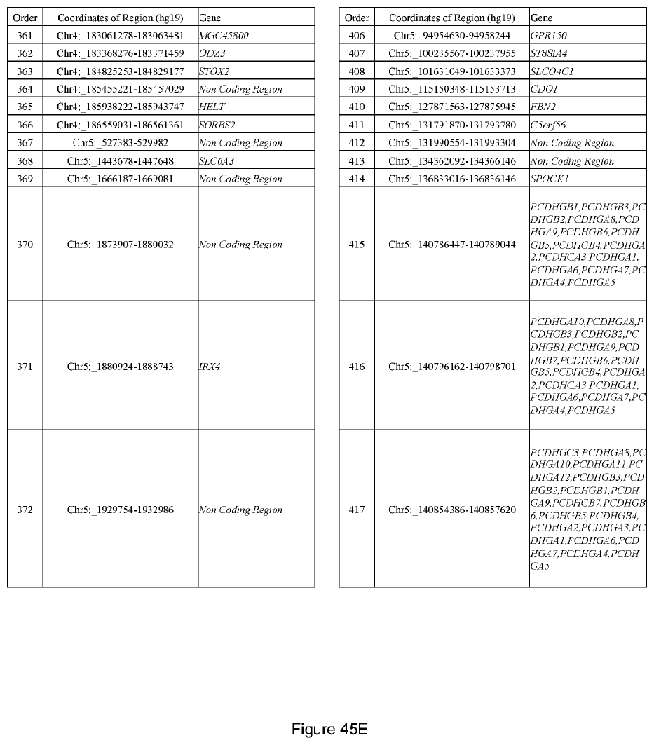

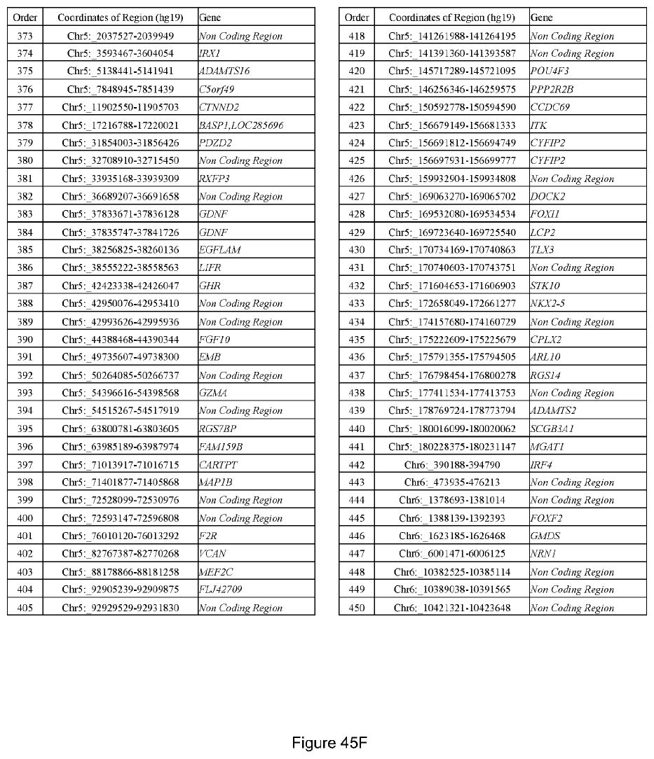

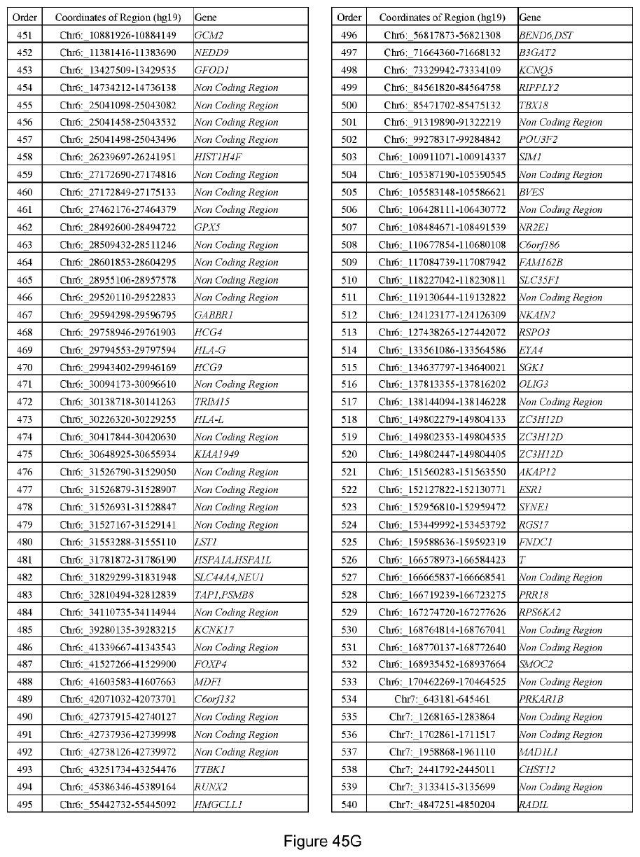

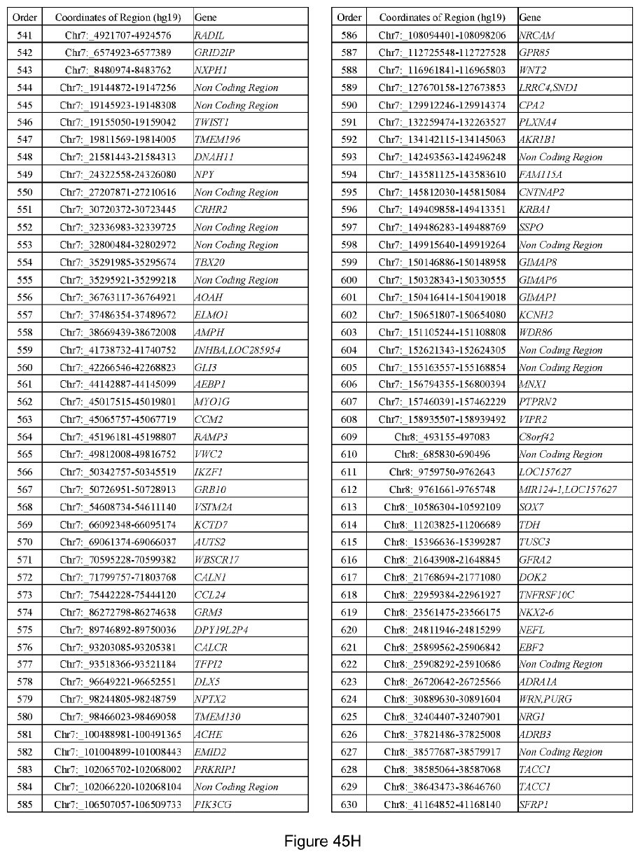

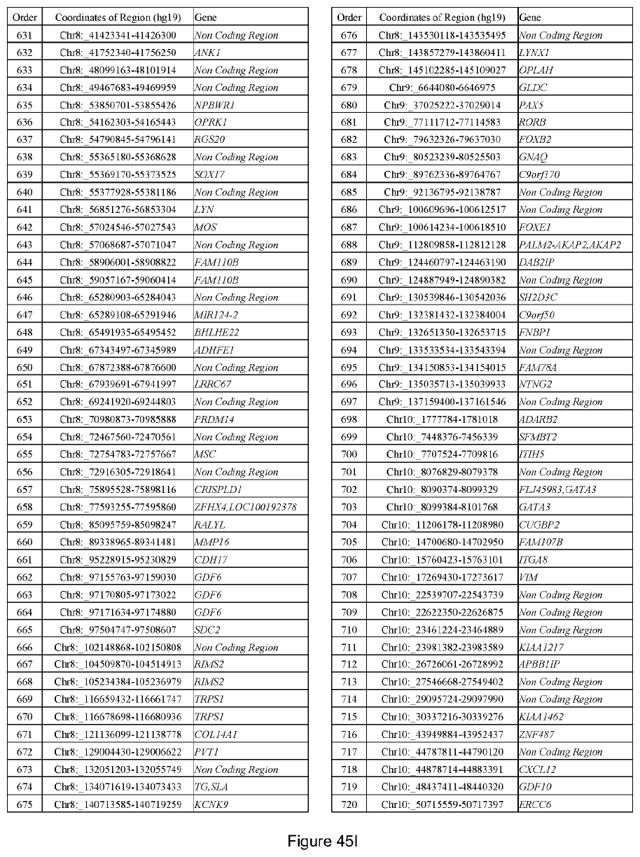

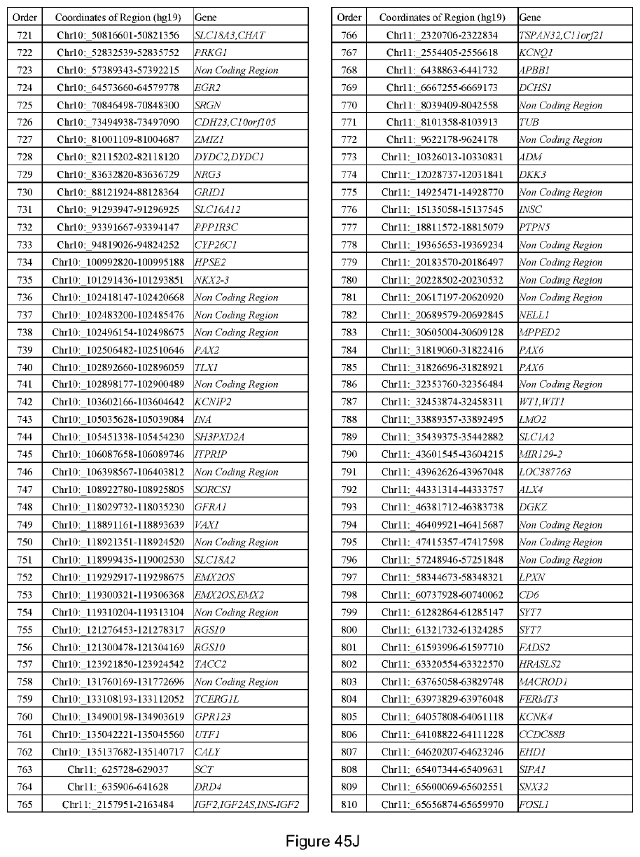

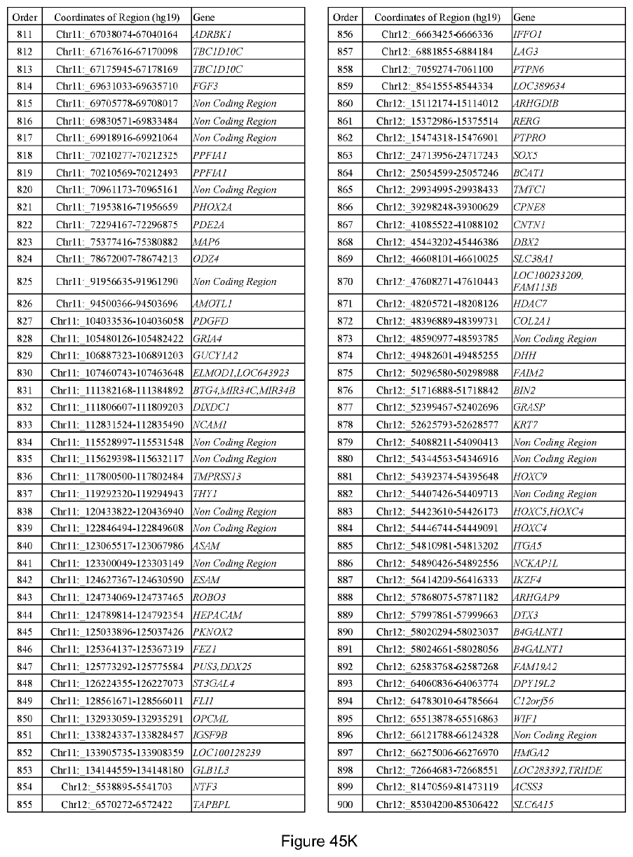

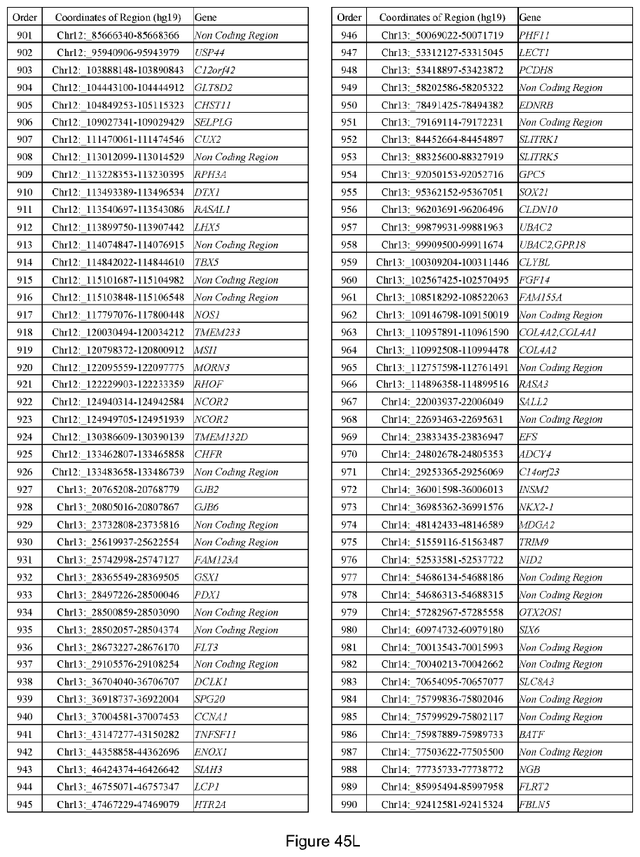

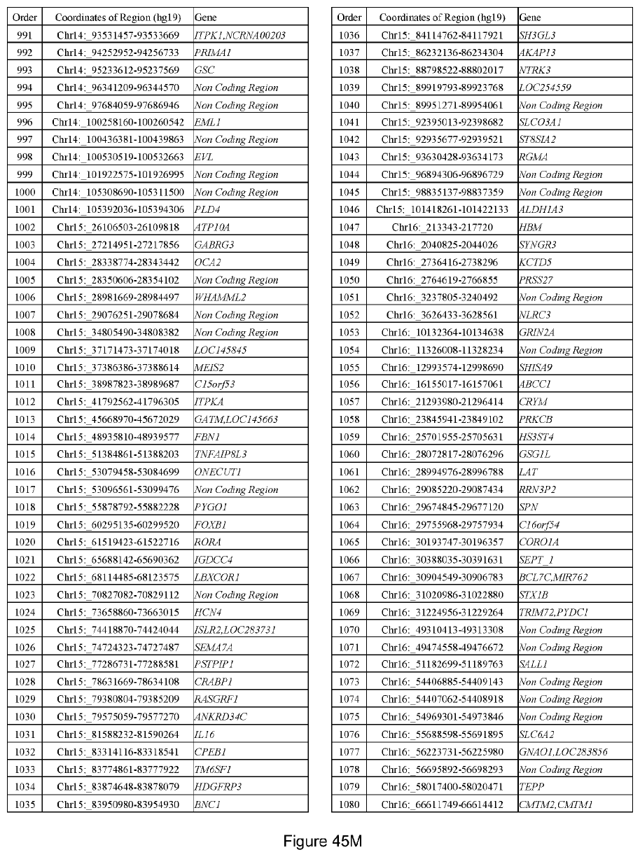

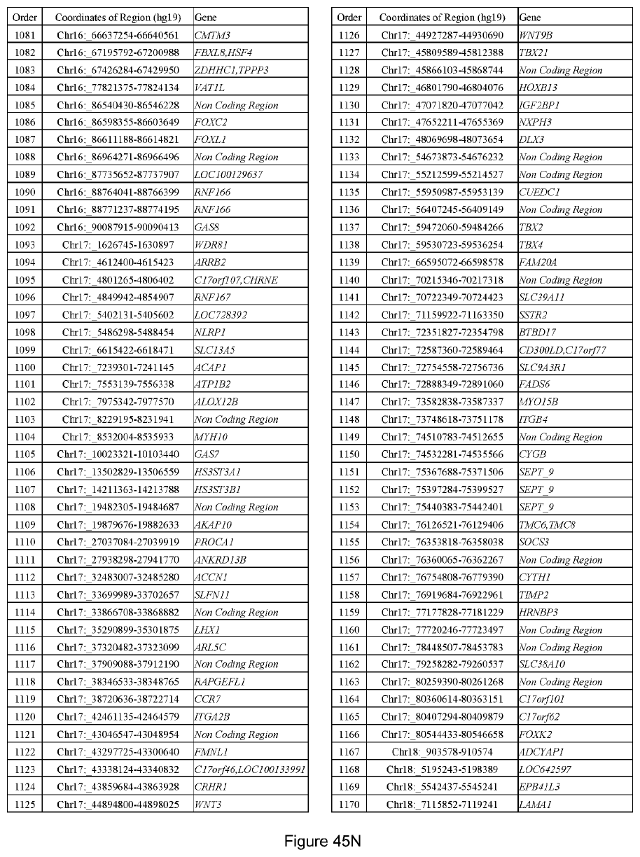

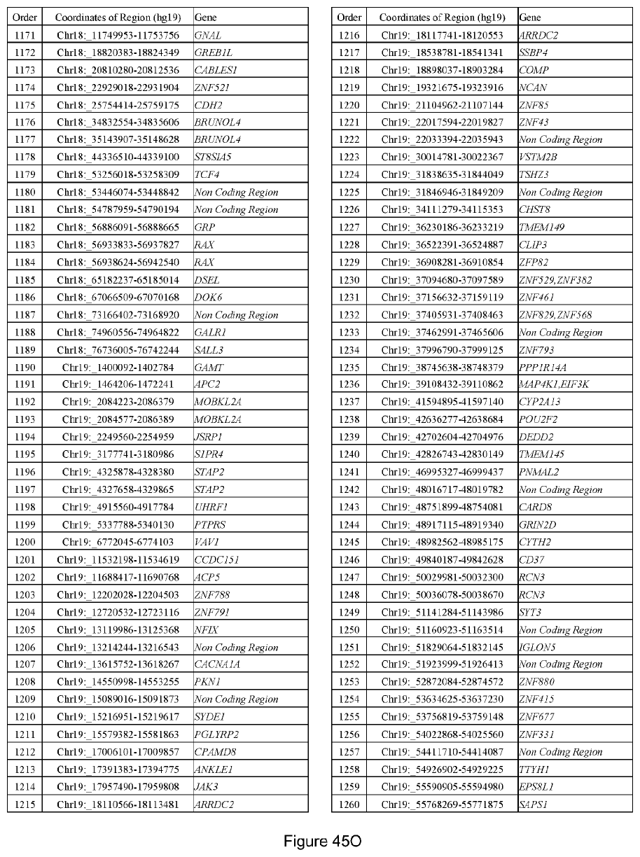

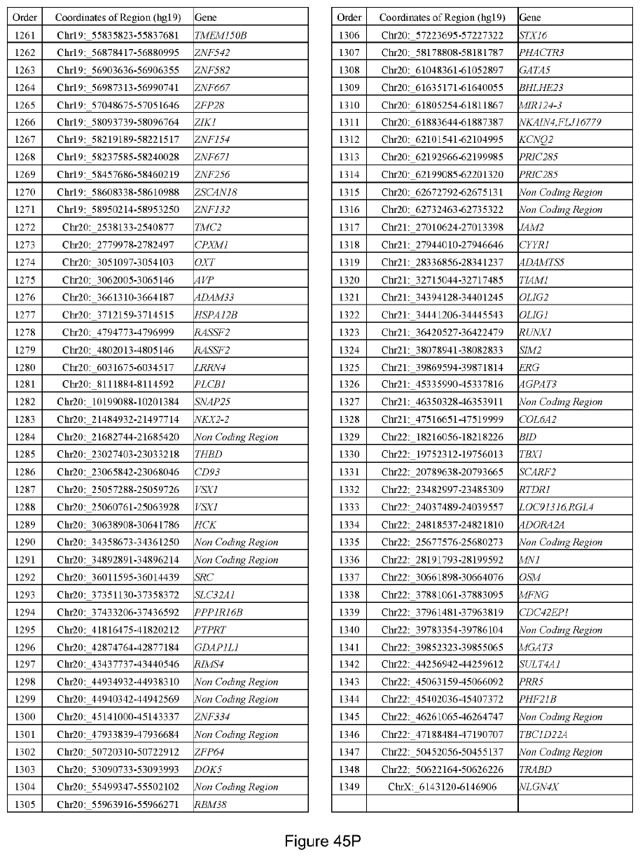

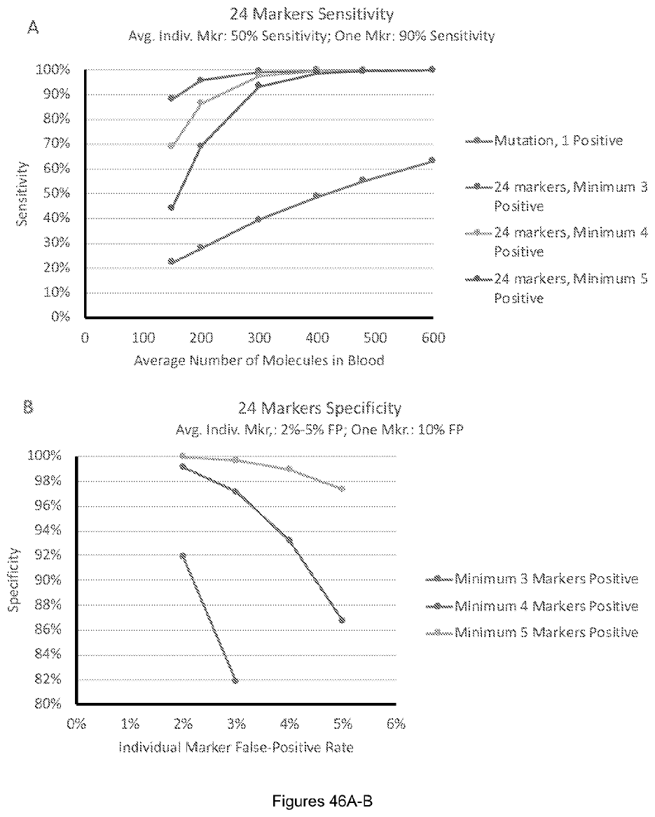

SEPT9 methylation is the basis for Epi proColon test, a CRC-detection assay by Epigenomics (Lofton-Day et al, “DNA Methylation Biomarkers for Blood-based Colorectal Cancer Screening,” Clinical Chemistry 54(2):414-423 (2008), which is hereby incorporated by reference in its entirety). While initial results on smaller sample sets showed promise, large-scale studies with 1,544 plasma samples showed a sensitivity of 64% for stage I-III CRC, and a specificity of 78%-82%, effectively sending 180 to 220 out of 1,000 individuals to unnecessary colonoscopies (Potter et al., “Validation of a Real-time PCR-based Qualitative Assay for the Detection of Methylated SEPT9 DNA in Human Plasma,” Clinical Chemistry 60(9):1183-1191 (2014), which is hereby incorporated by reference in its entirety). ClinicalGenomics is currently developing blood-based CRC detection test based on the methylation of the BCAT1 and IKZF1 genes (Pedersen et al., “Evaluation of an Assay for Methylated BCAT1 and IKZF1 in Plasma for Detection of Colorectal Neoplasia,” BMC Cancer 15:654 (2015), which is hereby incorporated by reference in its entirety). Large-scale studies using 2,105 plasma samples of this two-marker test showed an overall sensitivity of 66%, with 38% for stage I CRC, and an impressive specificity of 94%. Exact Sciences and collaborators have slightly improved the sensitivity of CRC fecal tests (Bosch et al., “Analytical Sensitivity and Stability of DNA Methylation Testing in Stool Samples for Colorectal Cancer Detection,” Cell Oncol. (Dordr) 35(4):309-315 (2012); Hong et al., “DNA Methylation Biomarkers of Stool and Blood for Early Detection of Colon Cancer,” Genet. Test. Mol. Biomarkers 17(5):401-406 (2013); Imperiale et al., “Multitarget Stool DNA Testing for Colorectal-Cancer Screening,” N. Engl. J. Med. 370(14):1287-1297 (2014); Xiao et al., “Validation of Methylation-Sensitive High-resolution Melting (MS-FIRM) for the Detection of Stool DNA Methylation in Colorectal Neoplasms,” Clin. Chim. Acta. 431:154-163 (2014); Yang et al., “Diagnostic Value of Stool DNA Testing for Multiple Markers of Colorectal Cancer and Advanced Adenoma: a Meta-Analysis,” Can. J. Gastroenterol. 27(8):467-475 (2013), which are hereby incorporated by reference in their entirety), by adding K-ras mutation as well as BMP3 and NDRG4 methylation markers (Lidgard et al., “Clinical Performance of an Automated Stool DNA Assay for Detection of Colorectal Neoplasia,” Clin. Gastroenterol. Hepatol. 11(10): 1313-1318 (2013), which is hereby incorporated by reference in its entirety). Epigenetic changes may mark not only the DNA (as methylation or hydroxy-methylation of promoter CpG sites) but also by appending methyl or acetyl groups on the histone proteins that bind to these promoters. These different epigenetic marks may be detected in circulating nucleosomes of colorectal cancer patients (Rahier et al., “Circulating Nucleosomes as New Blood-based Biomarkers for Detection of Colorectal Cancer,” Clin Epigenetics 9:53 (2017), which is hereby incorporated by reference in its entirety). The identification of blood-based, cancer-specific methylation markers has employed the entire TCGA Illumina 450K methylation datasets (consisting of primary tumors, matching normal for 33 types of cancer including CRC), along with additional methylation datasets (primary tumors, normal tissues, cell lines, peripheral blood, immune cells) from the Gene Expression Omnibus (GEO). In order to identify CRC-specific methylation markers, comparative statistical analyses of these datasets were used to identify candidate methylation markers with the following characteristics: highly methylated in CRC tissues and cell lines, unmethylated in normal colon, unmethylated in peripheral blood and immune infiltrates, unmethylated in most other cancer types. Validating the bioinformatic scheme, these methodologies also identified CpG sites previously reported to be hypermethylated in plasma from CRC patients (Church et al., “Prospective Evaluation of Methylated SEPT9 in Plasma for Detection of Asymptomatic Colorectal Cancer,” Gut 63(2):317-325 (2014); Lofton-Day et al., “DNA Methylation Biomarkers for Blood-based Colorectal Cancer Screening,” Clinical Chemistry 54(2):414-423 (2008); Toth et al., “Detection of Methylated SEPT9 in Plasma is a Reliable Screening Method for Both Left- and Right-sided Colon Cancers,” PloS One 7(9):e46000 (2012); Warren et al., “Septin 9 Methylated DNA is a Sensitive and Specific Blood Test for Colorectal Cancer,” BMC Medicine 9:133 (2011); Lange et al., “Genome-scale Discovery of DNA-methylation Biomarkers for Blood-based Detection of Colorectal Cancer,” PloS One 7(11):e50266 (2012); Margolin et al., “Robust Detection of DNA Hypermethylation of ZNF154 as a Pan-Cancer Locus with in Silico Modeling for Blood-Based Diagnostic Development,” The Journal of Molecular Diagnostics: JMD 18(2):283-298 (2016); Lin et al., “Clinical Relevance of Plasma DNA Methylation in Colorectal Cancer Patients Identified by Using a Genome-Wide High-Resolution Array,” Ann. Surg. Oncol. 22 Suppl 3:S1419-1427 (2015); Pedersen et al., “Evaluation of an Assay for Methylated BCAT1 and IKZF1 in Plasma for Detection of Colorectal Neoplasia,” BMC Cancer 15:654 (2015), which are hereby incorporated by reference in their entirety). To ensure that these methylation sites were specific to CRC and not a result of aging-related methylation (McClay et al., “A Methylome-wide Study of Aging Using Massively Parallel Sequencing of the Methyl-CpG-enriched Genomic Fraction From Blood in Over 700 subjects,” Hum. Mol. Genet. 23(5):1175-1185 (2014), which is hereby incorporated by reference in its entirety), the Pearson correlation was calculated between levels of methylation and patient age. Furthermore, hypermethylation of these sites did not significantly correlate with MSI status, implying that markers have been identified for all CRC subtypes. Overall, ˜10,000 tissue samples, >4 billion datapoints (datapoint=CpG percentage methylation per sample) were analyzed to identify an initial list of few hundred CRC-specific markers. CpG markers consistently show up in many types of cancer and are labeled as potential Pan-Oncology markers. Additional approaches for detecting low abundance 5 mC (or 5 hmC) are described in WO2016057832A2, which is hereby incorporated by reference in its entirety, or using other suitable means known in the art. FIG. 44 provides a list of primary CpG sites that are Colorectal Cancer and Colon-tissue specific markers, that may be used to identify the presence of colorectal cancer from cfDNA, or DNA within exosomes, or DNA in other protected states (such as within CTCs) within the blood. FIG. 45 provides a list of chromosomal regions or sub-regions within which are primary CpG sites that are Colorectal Cancer and Colon-tissue specific markers, that may be used to identify the presence of colorectal cancer from cfDNA, or DNA within exosomes, or DNA in other protected states (such as within CTCs) within the blood.

Mutation or methylation status may give a clear analytical cut-off, i.e. the assay either records a mutation or CpG methylation event, and false-positives are a consequence of biology, for example from age-related methylation. With other markers there may be a greater overlap between marker level for individuals with cancer and their matched normal controls, especially in attempting to identify cancer at the earliest stages. In such cases, cut-offs may be defined by “Z-score”, 2 standard deviations above normal values, or by setting the false-positive rate at an arbitrary level, i.e. 5% when evaluating a suitable set of age-matched normal samples. Generally, the set of age-matched normal should be suitably large enough to set cut-off of the marker-specific signal from a given disease sample at >85%; >90%; >95%; >96%; >97%; or >98% of the same marker-specific signals from the set of normal samples. The “Z-score” may be calculated using the formula: Z=(X−μ)/σ; where Z=Z-score, X=each value in the dataset, μ=mean of all values in the dataset, and σ=standard deviation of a sample. Likewise, when using the Z-score, the cut-off for marker-specific signal from a given disease sample may be set at a z-score of >1.03; >1.28; >1.65; >1.75; >1.88; or >2.05 compared to the same marker-specific signals from the set of normal samples. In some assays, marker levels, (i.e. DNA methylation levels for several gene promoter regions in plasma, or miRNA levels in urine) are quantified in relation to another marker, either internal or externally added in a qPCR reaction, where the cut-off is determined as a ΔCt value in the assay (Fackler et al., “Novel Methylated Biomarkers and a Robust Assay to Detect Circulating Tumor DNA in Metastatic Breast Cancer,” Cancer Res. 74(8):2160-70 (2014); U.S. Pat. No. 9,416,404 to Sukumar et al., which are hereby incorporated by reference in their entirety). Methylation status at defined promoter regions may also be determined using digital bisulfite genomic sequencing and digital MethyLight assays; using bisulfite conversion and preferential amplification of converted methylated sequences by blocking primers that interfere with amplification of converted unmethylated sequences; or depletion of unmethylated DNA using methyl-sensitive restriction endonucleases, followed by PCR (see U.S. Pat. No. 9,290,803 to Laird et al.; U.S. Pat. No. 9,476,100 to Frumkin, et al.; U.S. Pat. No. 9,765,397 to McEvoy et al.; U.S. Pat. No. 9,896,732 to Tabori et al.; U.S. Pat. No. 9,957,575 to Kottwitz et al., which are hereby incorporated by reference in their entirety).

The genome-wide methylation profile of cfDNA (known as the methylome) can be determined using next-generation sequencing, and the methylation pattern may be used to identify the presence of fetal, tumor, or other tissue DNA in the plasma (Sun et al., “Plasma DNA Tissue Mapping by Genome-wide Methylation Sequencing for Noninvasive Prenatal, Cancer, and Transplantation Assessments,” Proc. Natl. Acad. Sci. USA 112(40):E5503-12 (2015); Lehmann-Werman et al., “Identification of Tissue-specific Cell Death Using Methylation Patterns of Circulating DNA,” Proc. Natl. Acad. Sci. USA 113(13):E1826-34 (2016); U.S. Pat. No. 9,732,390 to Lo et al.; U.S. Pat. No. 9,984,201 to Zhang et al., which are hereby incorporated by reference in their entirety).

The aforementioned two-step screening assay sensitivities and specificities were calculated based on having an initial screen (with fewer markers) that cast a wide net to maximize sensitivity, followed by a second test (with more markers) on the initial presumptive positive samples, but the second test not only maintains the sensitivity, but also achieves high specificity to obtain a respectable positive predictive value. While colorectal cancer is a more frequent cancer, other cancers are less common, so achieving a good positive predictive value is critical to avoiding unnecessary follow-up diagnostic procedures. These initial calculations (in FIGS. 33-37) focused on methylation markers, with an average sensitivity of 50%, and individual marker false-positives ranging from 2%-5%, and average number of molecules in the blood set at 300 molecules. As a benchmark for these initial calculations, a mutation marker would give an overall sensitivity of 40%. In order to explore the influence of combining the methylation markers with other markers that may differ in both these values, additional calculations were performed, with an emphasis on potentially identifying the earliest (i.e. Stage I cancers), where the average number of molecules in the blood may be as low as 150 molecules. Four types of calculations were performed: (A) Average individual markers at 50% sensitivity and 2%-5% false-positives, with one marker (i.e. a protein marker) at 90% sensitivity but with 10% false-positives; (B) Average individual markers at 50% sensitivity and 2%-5% false-positives, with one marker at 80% sensitivity but with 15% false-positives; (C) Average individual markers at 50% sensitivity and 2%-5% false-positives, with two markers at 90% sensitivity each but with 10% false-positives each; and (D) Average individual markers at 50% sensitivity and 2%-5% false-positives, with two markers at 80% sensitivity each but with 15% false-positives each.

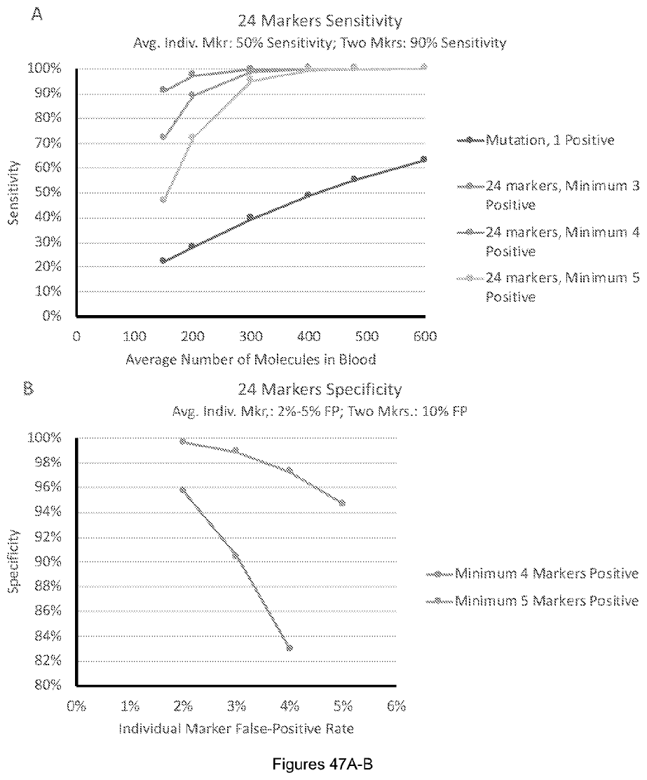

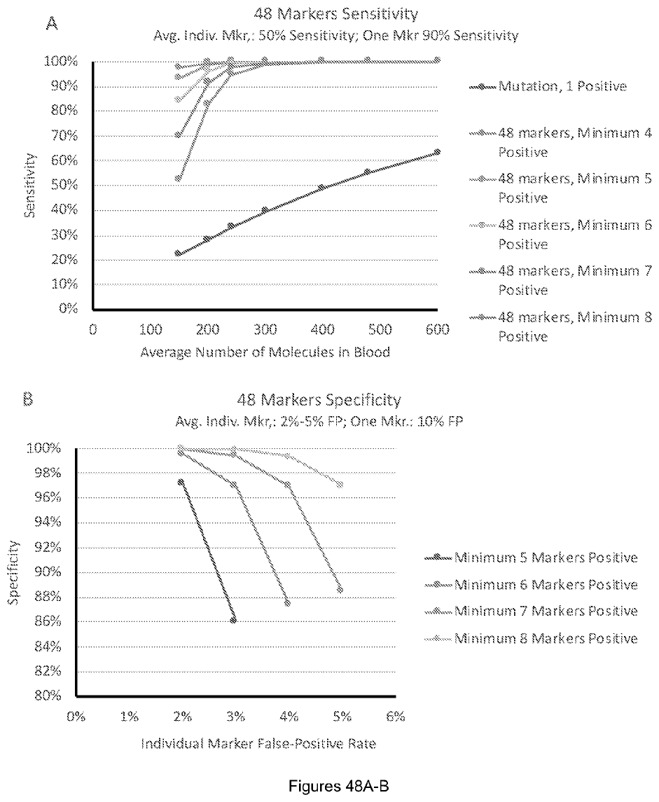

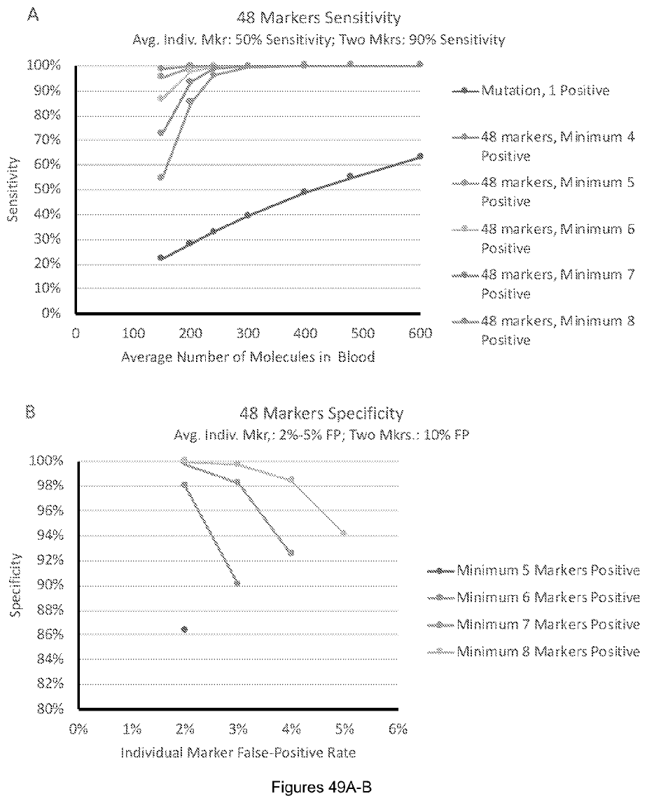

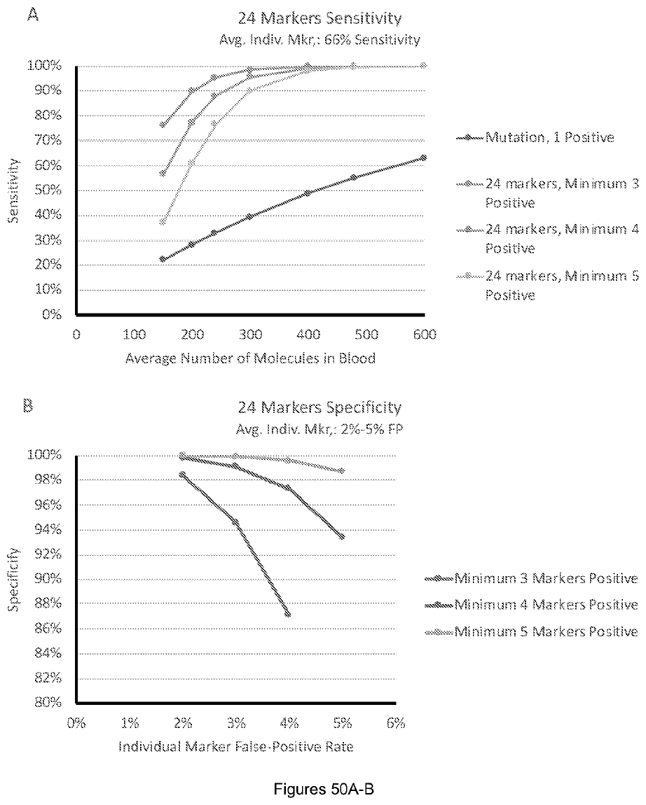

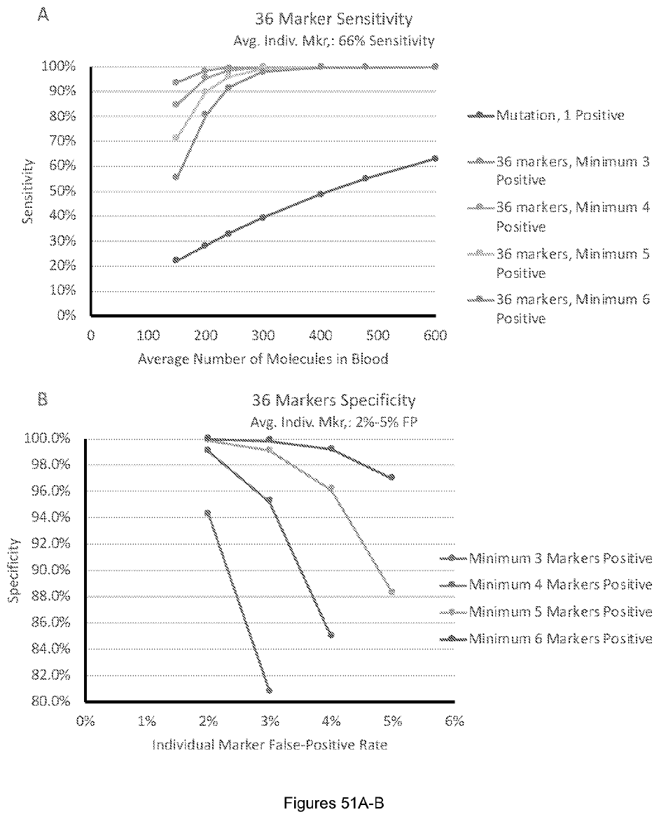

FIGS. 47-48 illustrate results for calculated overall Sensitivity and Specificity for 24 markers using conditions (A) and (C). The sensitivity curves provide overall sensitivity as a function of the average number of molecules in the blood for each marker, with separate curves for each minimum number of markers needed to call a sample as positive. The specificity curves provide overall specificity as a function of individual marker false-positive rates, again with separate curves for each minimum number of markers needed to call a sample as positive. The calculated numbers for overall Sensitivity and Specificity for 24 markers using the above 4 conditions are provided in the tables below.

TABLE 14
24 Markers Sensitivity
Avg. Indiv. Mkr,: 50% Sensitivity; One Mkr 90% Sensitivity

| Average | | | | |
|---|---|---|---|---|
| Number of | | 24 markers, | 24 markers, | 24 markers, |
| Molecules in | Mutation, 1 | Minimum 3 | Minimum 4 | Minimum 5 |
| Blood | Positive | Positive | Positive | Positive |
| 150 | 22.1% | 88.2% | 68.9% | 44.4% |
| 200 | 28.1% | 95.7% | 86.4% | 69.2% |
| 300 | 39.4% | 99.3% | 97.6% | 93.3% |
| 400 | 48.8% | 99.9% | 99.5% | 98.6% |
| 480 | 55.1% | 100.0% | 99.9% | 99.5% |
| 600 | 63.2% | 100.0% | 100.0% | 99.9% |

TABLE 15
24 Markers Specificity
Avg. Indiv. Mkr,: 2%-5% FP; One Mkr.: 10% FP

| Individual | Minimum 3 | Minimum 4 | Minimum 5 |
|---|---|---|---|
| marker FP | Markers | Markers | Markers |
| rate | Positive | Positive | Positive |
| 2% | 91.9% | 99.1% | 99.9% |
| 3% | 81.8% | 97.1% | 99.7% |
| 4% | | 93.2% | 98.9% |
| 5% | | 86.7% | 97.3% |

TABLE 16
24 Markers Sensitivity
Avg. Indiv. Mkr: 50% Sensitivity; One Mkr: 80% Sensitivity

| Average | | | | |
|---|---|---|---|---|
| Number of | | 24 markers, | 24 markers, | 24 markers, |
| Molecules in | Mutation, 1 | Minimum 3 | Minimum 4 | Minimum 5 |
| Blood | Positive | Positive | Positive | Positive |
| 150 | 22.1% | 84.8% | 65.1% | 41.5% |
| 200 | 28.1% | 93.5% | 83.1% | 65.6% |
| 300 | 39.4% | 98.7% | 96.2% | 90.9% |
| 400 | 48.8% | 99.7% | 99.1% | 97.7% |
| 480 | 55.1% | 99.9% | 99.7% | 99.2% |
| 600 | 63.2% | 100.0% | 100.0% | 99.8% |

TABLE 17
24 Markers Specificity
Avg. Indiv. Mkr,: 2%-5% FP; One Mkr.: 15% FP

| Individual | Minimum 3 | Minimum 4 | Minimum 5 |
|---|---|---|---|
| marker FP | Markers | Markers | Markers |
| rate | Positive | Positive | Positive |
| 2% | 87.9% | 98.7% | 99.9% |
| 3% | | 95.7% | 99.5% |
| 4% | | 89.8% | 98.4% |
| 5% | | 80.1% | 96.0% |

TABLE 18
24 Markers Sensitivity
Avg. Indiv. Mkr: 50% Sensitivity; Two Mkrs: 90% Sensitivity

| Average | | | | |
|---|---|---|---|---|
| Number of | | 24 markers, | 24 markers, | 24 markers, |
| Molecules in | Mutation, 1 | Minimum 3 | Minimum 4 | Minimum 5 |
| Blood | Positive | Positive | Positive | Positive |
| 150 | 22.1% | 90.9% | 71.9% | 46.7% |
| 200 | 28.1% | 97.4% | 89.0% | 72.0% |
| 300 | 39.4% | 99.8% | 98.8% | 95.2% |
| 400 | 48.8% | 100.0% | 99.9% | 99.4% |
| 480 | 55.1% | 100.0% | 100.0% | 99.8% |
| 600 | 63.2% | 100.0% | 100.0% | 100.0% |

TABLE 19
24 Markers Specificity
Avg. Indiv. Mkr,: 2%-5% FP; Two Mkrs.: 10% FP

| Individual | Minimum 3 | Minimum 4 | Minimum 5 |
|---|---|---|---|
| marker FP | Markers | Markers | Markers |
| rate | Positive | Positive | Positive |
| 2% | | 95.7% | 99.7% |
| 3% | | 90.4% | 98.9% |
| 4% | | 83.0% | 97.3% |
| 5% | | | 94.7% |

TABLE 20
24 Markers Sensitivity
Avg. Indiv. Mkr,: 50% Sensitivity; Two Mkrs: 80% Sensitivity

| Average | | | | |
|---|---|---|---|---|
| Number of | | 24 markers, | 24 markers, | 24 markers, |
| Molecules in | Mutation, 1 | Minimum 3 | Minimum 4 | Minimum 5 |
| Blood | Positive | Positive | Positive | Positive |
| 150 | 22.1% | 90.2% | 71.1% | 46.1% |
| 200 | 28.1% | 97.0% | 88.4% | 71.3% |
| 300 | 39.4% | 99.6% | 98.5% | 94.7% |
| 400 | 48.8% | 99.9% | 99.8% | 99.2% |
| 480 | 55.1% | 100.0% | 99.9% | 99.8% |
| 600 | 63.2% | 100.0% | 100.0% | 100.0% |

TABLE 21
24 Markers Specificity
Avg. Indiv. Mkr,: 2%-5% FP; Two Mkrs.: 15% FP

| Individual | Minimum 3 | Minimum 4 | Minimum 5 |
|---|---|---|---|
| marker FP | Markers | Markers | Markers |
| rate | Positive | Positive | Positive |
| 2% | | 90.4% | 99.2% |
| 3% | | | 97.4% |
| 4% | | | 93.9% |
| 5% | | | 88.0% |

Before evaluating what advantages, if any, there are to combining protein (or other markers) with methylation markers, an analysis is performed on the original 24 marker set, with average individual marker sensitivity at 50%, and 2%-5% false positive rates. In this example, if there is an average of 150 molecules in the blood for the earliest cancer (Stage I), and if that would cover at least one mutation, then the sensitivity for identifying such a cancer by next generation sequencing would be 22.1% (See FIG. 33A). If the individual marker FP rate is 3%, then if there is a 3-marker minimum, then overall FP rate is 5.4% for 24 markers, for a specificity of 94.6% (See FIG. 33B). At 3 markers, for Stage I cancer (at about 150 molecules of each positive marker in the blood), the test would miss 42.3%; i.e. for Stage I cancer the overall sensitivity would be 57.7% (See FIG. 33A). However, if the individual marker FP rate is 5%, then if there is a 4-marker minimum, then overall FP rate is 6.6% for 24 markers, for a specificity of 93.4% (See FIG. 33B). At 4 markers, for Stage I cancer (at about 150 molecules of each positive marker in the blood), the test would miss 64.7%; i.e. for Stage I cancer the sensitivity would be 35.3% (See FIG. 33A). While the specificity is reasonable, limiting the number of samples that would need to be retested in the second step of the assay, the assay would miss ⅔^{rd} of the earliest cancers.

The above numbers are compared to the graph in FIG. 46, i.e. condition (A), with average individual markers at 50% sensitivity and 2%-5% false-positives, with one marker (i.e. a protein marker) at 90% sensitivity but with 10% false-positives. While use of only 3 markers positive out of 24 markers provides a sensitivity of 88.2%, even with an individual marker 2% FP rate, the specificity would be 91.9%, if the FP rate were 3%, the overall specificity drops to 81.8%. This is the negative influence of the single marker with the high FP rate of 10%. Use of 4 markers positive out of 24 markers provides a sensitivity of 68.9%—still better than the original number of 57.7%, but now specificity improves to 97.1% with individual marker FP rates of 3%.

For condition (B), the average individual markers are at 50% sensitivity and 2%-5% false-positives, with one marker at 80% sensitivity but with 15% false-positives. Under these conditions, the specificity for 3 markers positive out of 24 markers would be at 87.9%, and thus would most likely not be used. Use of 4 markers positive out of 24 markers provides a sensitivity of 65.1%—still better than the original number of 57.7%, but now specificity improves to 95.7% with individual marker FP rates of 3%.

What if there are two markers with higher sensitivity (as well as higher FP rates)? For condition (C), with average individual markers at 50% sensitivity and 2%-5% false-positives, with two markers at 90% sensitivity each but with 10% false-positives each, see graph in FIG. 47. Under these conditions, the specificity for 3 markers positive out of 24 markers would be below 80%, and thus would not be used. Use of 4 markers positive out of 24 markers provides a sensitivity of 71.9%—still better than the original number of 57.7%, but now specificity is at 95.7% with individual marker FP rates of 2%. Should the individual marker FP rates rise to 3%, then overall specificity drops to 90.4%.

For condition (D), the average individual markers are at 50% sensitivity and 2%-5% false-positives, with two markers at 80% sensitivity each but with 15% false-positives each. Under these conditions, the specificity for 3 markers positive out of 24 markers would be below 80%, and thus would not be used. Use of 4 markers positive out of 24 markers provides a sensitivity of 71.1%—still better than the original number of 57.7%, but now specificity is at 90.4% with individual marker FP rates of 2%. Should the individual marker FP rates rise to 3%, then 5 markers would be required, and while overall specificity would rise to 97.4%, the sensitivity would drop to 46.1%, which is worse than the original number of 57.7%.

Thus, from analysis of the above 4 conditions (A-D), condition (C) provided the best improvement in overall sensitivity (71.9%) for detecting Stage I cancer, while still keeping overall specificity reasonable (95.7%) for the initial 24 marker screen, should it now include two markers with higher sensitivity (90%), but worse FP rate of 10% for each of these markers.

The calculated numbers for two of the overall Sensitivity and Specificity for 36 markers using two of the aforementioned 4 conditions: (A) Average individual markers at 50% sensitivity and 2%-5% false-positives, with one marker (i.e. a protein marker) at 90% sensitivity but with 10% false-positives; and (C) Average individual markers at 50% sensitivity and 2%-5% false-positives, with two markers at 90% sensitivity each but with 10% false-positives each—are provided in the tables below. The sensitivity curves provide overall sensitivity as a function of the average number of molecules in the blood for each marker, with separate curves for each minimum number of markers needed to call a sample as positive. The specificity curves provide overall specificity as a function of individual marker false-positive rates, again with separate curves for each minimum number of markers needed to call a sample as positive. The calculated numbers for overall Sensitivity and Specificity for 36 markers using the above two conditions are provided in the tables below.

TABLE 22
36 Markers Sensitivity
Avg. Indiv. Mkr,: 50% Sensitivity; One Mkr 90% Sensitivity

| Average | | | | | |
|---|---|---|---|---|---|
| Number of | | 36 markers, | 36 markers, | 36 markers, | 36 markers, |
| Molecules in | Mutation, 1 | Minimum 3 | Minimum 4 | Minimum 5 | Minimum 6 |
| Blood | Positive | Positive | Positive | Positive | Positive |
| 150 | 22.1% | 96.8% | 89.2% | 73.9% | 53.2% |
| 200 | 28.1% | 99.3% | 97.6% | 93.3% | 84.1% |
| 240 | 33.0% | 99.7% | 99.1% | 97.4% | 93.4% |
| 300 | 39.4% | 99.9% | 99.8% | 99.3% | 98.3% |
| 400 | 48.8% | 100.0% | 100.0% | 99.9% | 99.8% |
| 480 | 55.1% | 100.0% | 100.0% | 100.0% | 100.0% |
| 600 | 63.2% | 100.0% | 100.0% | 100.0% | 100.0% |

TABLE 23
36 Markers Specificity
Avg. Indiv. Mkr,: 2%-5% FP; One Mkr.: 10% FP

| | Minimum | Minimum | Minimum | Minimum |
|---|---|---|---|---|
| Individual | 4 | 5 | 6 | 7 |
| marker FP | Markers | Markers | Markers | Markers |
| rate | Positive | Positive | Positive | Positive |
| 2% | 95.3% | 99.4% | 99.9% | 100.0% |
| 3% | 84.1% | 96.9% | 99.5% | 99.9% |
| 4% | | 90.3% | 98.0% | 99.7% |
| 5% | | | 93.9% | 98.7% |

TABLE 24
36 Markers Sensitivity
Avg. Indiv. Mkr: 50% Sensitivity; Two Mkrs: 90% Sensitivity

| | | 36 | 36 | 36 | 36 |
|---|---|---|---|---|---|
| Average | | markers, | markers, | markers, | markers, |
| Number of | Mutation, | Minimum | Minimum | Minimum | Minimum |
| Molecules | 1 | 3 | 4 | 5 | 6 |
| in Blood | Positive | Positive | Positive | Positive | Positive |
| 150 | 22.1% | 98.1% | 91.3% | 76.3% | 55.3% |
| 200 | 28.1% | 99.8% | 98.8% | 95.2% | 86.7% |
| 240 | 33.0% | 99.9% | 99.7% | 98.6% | 95.2% |
| 300 | 39.4% | 100.0% | 99.9% | 99.8% | 99.1% |
| 400 | 48.8% | 100.0% | 100.0% | 100.0% | 99.9% |
| 480 | 55.1% | 100.0% | 100.0% | 100.0% | 100.0% |
| 600 | 63.2% | 100.0% | 100.0% | 100.0% | 100.0% |

TABLE 25
36 Markers Specificity
Avg. Indiv. Mkr,: 2%-5% FP; Two Mkrs.: 10% FP

| | Minimum | Minimum | Minimum | Minimum |
|---|---|---|---|---|
| Individual | 4 | 5 | 6 | 7 |
| marker FP | Markers | Markers | Markers | Markers |
| rate | Positive | Positive | Positive | Positive |
| 2% | | 97.0% | 99.7% | 100.0% |
| 3% | | 89.8% | 98.4% | 99.8% |
| 4% | | | 95.0% | 99.1% |
| 5% | | | 87.8% | 97.4% |

What are the advantages to combining protein (or other markers) with methylation markers, an analysis is performed on the original 36 marker set, with average individual marker sensitivity at 50%, and 2%-5% false positive rates? In this example, if there is an average of 150 molecules in the blood for the earliest cancer (Stage I), and if that would cover at least one mutation, then the sensitivity for identifying such a cancer by next generation sequencing would be 22.1% (See FIG. 34A). If the individual marker FP rate is 2%, then if there is a 3-marker minimum, then overall FP rate is 5.7% for 36 markers, for a specificity of 94.3% (See FIG. 34B). At 3 markers, for Stage I cancer (at about 150 molecules of each positive marker in the blood), the test would miss 17.4%; i.e. for Stage I cancer the overall sensitivity would be 82.6% (See FIG. 34A). However, if the individual marker FP rate is 3%, then if there is a 4-marker minimum, then overall FP rate is 4.8% for 36 markers, for a specificity of 95.2% (See FIG. 34B). At 4 markers, for Stage I cancer (at about 150 molecules of each positive marker in the blood), the test would miss 34.1%; i.e. for Stage I cancer the sensitivity would be 65.8% (See FIG. 34A). While the specificity is reasonable, limiting the number of samples that would need to be retested in the second step of the assay, the assay would miss ⅓^{rd} of the earliest cancers.

The above numbers are compared to the results for condition (A), with average individual markers at 50% sensitivity and 2%-5% false-positives, with one marker (i.e. a protein marker) at 90% sensitivity but with 10% false-positives. Use of only 4 markers positive out of 36 markers provides a sensitivity of 89.2%, and with an individual marker 2% FP rate, the specificity would be 95.3%. If the FP rate were 3%, this would require use of 5 markers positive out of 36 markers to provide a sensitivity of 73.9%—still better than the original number of 65.8%, but now specificity improves to 96.9% with individual marker FP rates of 3%.

For condition (C), the average individual markers are at 50% sensitivity and 2%-5% false-positives, with two markers at 90% sensitivity each but with 10% false-positives each. Under these conditions, use of 5 markers positive out of 36 markers provides a sensitivity of 76.3%—still better than the original number of 65.8%, but now specificity is at 97.0% with individual marker FP rates of 2%. Should the individual marker FP rates rise to 3%, then overall specificity drops to 89.8%.

Thus, from analysis of the above conditions (A, C), condition (C) provided the best improvement in overall sensitivity (76.3%) for detecting Stage I cancer, while still keeping overall specificity reasonable (97.0%) for the initial 36 marker screen, should it now include two markers with higher sensitivity (90%), but worse FP rate of 10% for each of these markers.

FIGS. 49-50 illustrate results for calculated overall Sensitivity and Specificity for 48 markers using the aforementioned 2 conditions: (A) Average individual markers at 50% sensitivity and 2%-5% false-positives, with one marker (i.e. a protein marker) at 90% sensitivity but with 10% false-positives; and (C) Average individual markers at 50% sensitivity and 2%-5% false-positives, with two markers at 90% sensitivity each but with 10% false-positives each. The sensitivity curves provide overall sensitivity as a function of the average number of molecules in the blood for each marker, with separate curves for each minimum number of markers needed to call a sample as positive. The specificity curves provide overall specificity as a function of individual marker false-positive rates, again with separate curves for each minimum number of markers needed to call a sample as positive. The calculated numbers for overall Sensitivity and Specificity for 48 markers using the above two conditions are provided in the tables below.

TABLE 26
48 Markers Sensitivity
Avg. Indiv. Mkr,: 50% Sensitivity; One Mkr 90% Sensitivity

| Average | | | | | | |
|---|---|---|---|---|---|---|
| Number of | | 48 markers, | 48 markers, | 48 markers, | 48 markers, | 48 markers, |
| Molecules | Mutation, 1 | Minimum 4 | Minimum 5 | Minimum 6 | Minimum 7 | Minimum 8 |
| in Blood | Positive | Positive | Positive | Positive | Positive | Positive |
| 150 | 22.1% | 97.6% | 93.3% | 84.2% | 69.7% | 52.1% |
| 200 | 28.1% | 99.5% | 98.6% | 96.4% | 91.5% | 82.7% |
| 240 | 33.0% | 99.9% | 99.7% | 99.1% | 97.7% | 94.7% |
| 300 | 39.4% | 100.0% | 99.9% | 99.8% | 99.5% | 98.9% |
| 400 | 48.8% | 100.0% | 100.0% | 100.0% | 100.0% | 99.9% |
| 480 | 55.1% | 100.0% | 100.0% | 100.0% | 100.0% | 100.0% |
| 600 | 63.2% | 100.0% | 100.0% | 100.0% | 100.0% | 100.0% |

TABLE 27
48 Markers Specificity
Avg. Indiv. Mkr,: 2%-5% FP; One Mkr.: 10% FP

| | Minimum | Minimum | Minimum | Minimum |
|---|---|---|---|---|
| Individual | 5 | 6 | 7 | 8 |
| marker FP | Markers | Markers | Markers | Markers |
| rate | Positive | Positive | Positive | Positive |
| 2% | 97.3% | 99.6% | 100.0% | 100.0% |
| 3% | 86.1% | 97.0% | 99.5% | 99.9% |
| 4% | | 87.4% | 97.0% | 99.4% |
| 5% | | | 88.5% | 97.1% |

TABLE 28
48 Markers Sensitivity
Avg. Indiv. Mkr: 50% Sensitivity; Two Mkrs: 90% Sensitivity

| Average | | | | | | |
|---|---|---|---|---|---|---|
| Number of | | 48 markers, | 48 markers, | 48 markers, | 48 markers, | 48 markers, |
| Molecules | Mutation, 1 | Minimum 4 | Minimum 5 | Minimum 6 | Minimum 7 | Minimum 8 |
| in Blood | Positive | Positive | Positive | Positive | Positive | Positive |
| 150 | 22.1% | 98.8% | 95.2% | 86.7% | 72.4% | 54.4% |
| 200 | 28.1% | 99.9% | 99.4% | 97.8% | 93.5% | 85.2% |
| 240 | 33.0% | 100.0% | 99.9% | 99.6% | 98.7% | 96.2% |
| 300 | 39.4% | 100.0% | 100.0% | 99.9% | 99.8% | 99.5% |
| 400 | 48.8% | 100.0% | 100.0% | 100.0% | 100.0% | 100.0% |
| 480 | 55.1% | 100.0% | 100.0% | 100.0% | 100.0% | 100.0% |
| 600 | 63.2% | 100.0% | 100.0% | 100.0% | 100.0% | 100.0% |

TABLE 29
48 Markers Specificity
Avg. Indiv. Mkr,: 2%-5% FP; Two Mkrs.: 10% FP

| | Minimum | Minimum | Minimum | Minimum |
|---|---|---|---|---|
| Individual | 5 | 6 | 7 | 8 |
| marker FP | Markers | Markers | Markers | Markers |
| rate | Positive | Positive | Positive | Positive |
| 2% | 86.3% | 98.0% | 99.8% | 100.0% |
| 3% | | 90.1% | 98.2% | 99.7% |
| 4% | | | 92.5% | 98.5% |
| 5% | | | | 94.1% |

What are the advantages to combining protein (or other markers) with methylation markers, an analysis is performed on the original 48-marker set, with average individual marker sensitivity at 50%, and 2%-5% false positive rates? In this example, if there is an average of 150 molecules in the blood for the earliest cancer (Stage I), and if that would cover at least one mutation, then the sensitivity for identifying such a cancer by next generation sequencing would be 22.1% (See FIG. 35A). If the individual marker FP rate is 2%, then if there is a 4-marker minimum, then overall FP rate is 3.1% for 48 markers, for a specificity of 96.9% (See FIG. 35B). At 4 markers, for Stage I cancer (at about 150 molecules of each positive marker in the blood), the test would miss 15.1%; i.e. for Stage I cancer the overall sensitivity would be 84.9% (See FIG. 35A). However, if the individual marker FP rate is 3%, then if there is a 5-marker minimum, then overall FP rate is 4.2% for 48 markers, for a specificity of 95.8% (See FIG. 35B). At 5 markers minimum, for Stage I cancer (at about 150 molecules of each positive marker in the blood), the test would miss 28.4%; i.e. for Stage I cancer the sensitivity would be 71.6% (See FIG. 35A). While the specificity is reasonable, limiting the number of samples that would need to be retested in the second step of the assay, the assay would miss a little over ¼^{th} of the earliest cancers.

The above numbers are compared to the graph in FIG. 48, i.e. condition (A), with average individual markers at 50% sensitivity and 2%-5% false-positives, with one marker (i.e. a protein marker) at 90% sensitivity but with 10% false-positives. Use of only 5 markers positive out of 48 markers provides a sensitivity of 93.3%, and with an individual marker 2% FP rate, the specificity would be 97.3%. If the FP rate were 3%, this would require use of 6 markers positive out of 48 markers to provide a sensitivity of 84.2%—still better than the original number of 71.6%, but now specificity improves to 97.0% with individual marker FP rates of 3%.

For condition (C), with average individual markers at 50% sensitivity and 2%-5% false-positives, with two markers at 90% sensitivity each but with 10% false-positives each, see graph in FIG. 49. Under these conditions, use of 5 markers positive out of 48 markers provides a sensitivity of 90.9%—still better than the original number of 71.6%, but now specificity is at 97.0% with individual marker FP rates of 2%. If the FP rate were 3%, this would require use of 6 markers positive out of 48 markers to provide a sensitivity of 81.0%—still better than the original number of 71.6%, but now specificity changes to 95.5% with individual marker FP rates of 3%.

From the above charts, the receiver operating characteristic (ROC) curves may be calculated by plotting Sensitivity vs. 1-Specificity. Since these are theoretical calculations, the curves were generated for different levels of average marker false-positive rates of 2%, 3%, 4%, and 5%. The AUC (Area under curve) were calculated for ROC curves for 24 markers, with average individual marker at 50% Sensitivity with 2%-3% FP, and one marker at 90% Sensitivity with 10% FP; 24 markers, with average individual marker at 50% Sensitivity with 2%-3% FP, and two markers at 90% Sensitivity with 10% FP; 36 markers, with average individual marker at 50% Sensitivity with 2%-3% FP, and one marker at 90% Sensitivity with 10% FP; 36 markers, with average individual marker at 50% Sensitivity with 2%-3% FP, and two markers at 90% Sensitivity with 10% FP; 48 markers, with average individual marker at 50% Sensitivity with 2%-3% FP, and one marker at 90% Sensitivity with 10% FP; and 48 markers, with average individual marker at 50% Sensitivity with 2%-3% FP, and two markers at 90% Sensitivity with 10% FP; and are provided in the Tables below. Using the benchmark of an average of 150 molecules in the blood for the earliest cancer (Stage I), and looking only at the 3% individual marker FP rate AUC values are at 77% with 24 markers (average individual marker at 50% Sensitivity), improve to 91% with 24 markers (average individual marker at 50% Sensitivity, and one marker at 90% Sensitivity with 10% FP), but decrease to 83% with 24 markers (average individual marker at 50% Sensitivity, and two markers at 90% Sensitivity with 10% FP); AUC values are at 87% with 36 markers (average individual marker at 50% Sensitivity), improve to 91% with 36 markers (average individual marker at 50% Sensitivity, and one marker at 90% Sensitivity with 10% FP), but decrease to 85% with 36 markers (average individual marker at 50% Sensitivity, and two markers at 90% Sensitivity with 10% FP); and AUC values are at 89% with 48 markers (average individual marker at 50% Sensitivity), improve to 91% with 48 markers (average individual marker at 50% Sensitivity, and one marker at 90% Sensitivity with 10% FP), and improve slightly to 92% with 48 markers (average individual marker at 50% Sensitivity, and two markers at 90% Sensitivity with 10% FP). These results illustrate that for multiple marker assays achieving good sensitivities and specificities for the earliest cancers is aided by having a single marker with above average sensitivities (i.e. 90%), even at the cost of a higher false-positive rate (i.e. 10%). There is no major benefit in sensitivity increasing the number of markers in the first step of the assay from 24 to 36 to 48 markers—but the increase in markers does improve specificity, which is important in limiting the number of samples that undergo the second step of the test.

TABLE 30
24, 36, & 48 Marker AUC Values from ROC Curves;
Avg. Indiv. Mkr,: 50% Sensitivity; One Mkr: 90% Sensitivity at 10% FP

| Total | | | | | | | |
|---|---|---|---|---|---|---|---|
| Markers: | | | | | | | |
| Individual | | | | | | | |
| marker | 150 | 200 | 240 | 300 | 400 | 480 | 600 |
| FP rate | Molecules | Molecules | Molecules | Molecules | Molecules | Molecules | Molecules |
| 24 Mkrs: 2% | 93% | 97% | | 99% | >99% | >99% | >99% |
| 24 Mkrs: 3% | 91% | 96% | | 99% | >99% | >99% | >99% |
| 35 Mkrs: 2% | 94% | 99% | >99% | >99% | >99% | >99% | >99% |
| 35 Mkrs: 3% | 91% | 96% | 99% | >99% | >99% | >99% | >99% |
| 48 Mkrs: 2% | 96% | 99% | >99% | >99% | >99% | >99% | >99% |
| 48 Mkrs: 3% | 91% | 96% | 99% | >99% | >99% | >99% | >99% |

TABLE 31
24, 36, & 48 Marker AUC Values from ROC Curves;
Avg. Indiv. Mkr,: 50% Sensitivity; Two Mkrs: 90% Sensitivity at 10% FP

| Total | | | | | | | |
|---|---|---|---|---|---|---|---|
| Markers: | | | | | | | |
| Individual | | | | | | | |
| marker | 150 | 200 | 240 | 300 | 400 | 480 | 600 |
| FP rate | Molecules | Molecules | Molecules | Molecules | Molecules | Molecules | Molecules |
| 24 Mkrs: 2% | 85% | 94% | | 99% | >99% | >99% | >99% |
| 24 Mkrs: 3% | 83% | 93% | | 99% | >99% | >99% | >99% |
| 36 Mkrs: 2% | 87% | 97% | 99% | >99% | >99% | >99% | >99% |
| 36 Mkrs: 3% | 85% | 96% | 98% | 99% | >99% | >99% | >99% |
| 48 Mkrs: 2% | 96% | 99% | >99% | >99% | >99% | >99% | >99% |
| 48 Mkrs: 3% | 92% | 98% | >99% | >99% | >99% | >99% | >99% |

While the above calculations are based on increasing the sensitivity of one or two markers, what if the average sensitivity of individual markers was increased from 50% to 66%? FIGS. 51-53 illustrate results for calculated overall Sensitivity and Specificity for 24, 36, and 48 markers, respectively. These graphs are based on the assumption that the average individual marker sensitivity is 66%, and the average individual marker false-positive rate is from 2% to 5%. The sensitivity curves provide overall sensitivity as a function of the average number of molecules in the blood for each marker, with separate curves for each minimum number of markers needed to call a sample as positive. The specificity curves provide overall specificity as a function of individual marker false-positive rates, again with separate curves for each minimum number of markers needed to call a sample as positive. The calculated numbers for overall Sensitivity and Specificity for 24, 36, and 48 markers, respectively, where the average individual marker sensitivity is 50% (as described previously) or 66% are provided in the tables below.

TABLE 32
24 Markers Sensitivity;
Avg. Indiv. Mkr,: 50% Sensitivity

| | | 24 | 24 | 24 |
|---|---|---|---|---|
| Average | | markers, | markers, | markers, |
| Number of | Mutation, | Minimum | Minimum | Minimum |
| Molecules | 1 | 3 | 4 | 5 |
| in Blood | Positive | Positive | Positive | Positive |
| 150 | 22.1% | 57.7% | 35.3% | 18.5% |
| 200 | 28.1% | 76.2% | 56.7% | 37.1% |
| 240 | 33.0% | 85.7% | 70.6% | 52.4% |
| 300 | 39.4% | 93.8% | 84.9% | 71.5% |
| 400 | 48.8% | 98.6% | 95.8% | 90.0% |
| 480 | 55.1% | 99.6% | 98.6% | 96.2% |
| 600 | 63.2% | 99.9% | 99.8% | 99.2% |

TABLE 33
24 Markers Sensitivity;
Avg. Indiv. Mkr,: 66% Sensitivity

| | | 24 | 24 | 24 |
|---|---|---|---|---|
| Average | | markers, | markers, | markers, |
| Number of | Mutation, | Minimum | Minimum | Minimum |
| Molecules | 1 | 3 | 4 | 5 |
| in Blood | Positive | Positive | Positive | Positive |
| 150 | 22.1% | 76.2% | 56.7% | 37.1% |
| 200 | 28.1% | 89.8% | 77.5% | 61.0% |
| 240 | 33.0% | 95.4% | 88.1% | 76.5% |
| 300 | 39.4% | 98.6% | 95.8% | 90.0% |
| 400 | 48.8% | 99.8% | 99.3% | 98.0% |
| 480 | 55.1% | 100.0% | 99.9% | 99.6% |
| 600 | 63.2% | 100.0% | 100.0% | 100.0% |

TABLE 34
24 Marker Specificity

| | Minimum | Minimum | Minimum |
|---|---|---|---|
| Individual | 3 | 4 | 5 |
| marker FP | Markers | Markers | Markers |
| rate | Positive | Positive | Positive |
| 2% | 98.4% | 99.8% | 99.9% |
| 3% | 94.6% | 99.1% | 99.9% |
| 4% | 87.1% | 97.3% | 99.6% |
| 5% | | 93.4% | 98.7% |

TABLE 35
36 Marker Sensitivity;
Avg. Indiv. Mkr,: 50% Sensitivity

| | | 36 | 36 | 36 | 36 |
|---|---|---|---|---|---|
| Average | | markers, | markers, | markers, | markers, |
| Number of | Mutation, | Minimum | Minimum | Minimum | Minimum |
| Molecules | 1 | 3 | 4 | 5 | 6 |
| in Blood | Positive | Positive | Positive | Positive | Positive |
| 150 | 22.1% | 82.6% | 65.8% | 46.8% | 29.7% |
| 200 | 28.1% | 93.8% | 84.9% | 71.5% | 55.4% |
| 240 | 33.0% | 97.5% | 92.8% | 84.4% | 72.4% |
| 300 | 39.4% | 99.4% | 97.9% | 94.5% | 88.4% |
| 400 | 48.8% | 99.9% | 99.8% | 99.2% | 98.0% |
| 480 | 55.1% | 100.0% | 100.0% | 99.9% | 99.6% |
| 600 | 63.2% | 100.0% | 100.0% | 100.0% | 100.0% |

TABLE 36
36 Marker Sensitivity;
Avg. Indiv. Mkr,: 66% Sensitivity

| | | 36 | 36 | 36 | 36 |
|---|---|---|---|---|---|
| Average | | markers, | markers, | markers, | markers, |
| Number of | Mutation, | Minimum | Minimum | Minimum | Minimum |
| Molecules | 1 | 3 | 4 | 5 | 6 |
| in Blood | Positive | Positive | Positive | Positive | Positive |
| 150 | 22.1% | 93.8% | 84.9% | 71.5% | 55.4% |
| 200 | 28.1% | 98.6% | 95.8% | 90.0% | 80.9% |
| 240 | 33.0% | 99.6% | 98.6% | 96.2% | 91.6% |
| 300 | 39.4% | 99.9% | 99.8% | 99.2% | 98.0% |
| 400 | 48.8% | 100.0% | 100.0% | 100.0% | 99.9% |
| 480 | 55.1% | 100.0% | 100.0% | 100.0% | 100.0% |
| 600 | 63.2% | 100.0% | 100.0% | 100.0% | 100.0% |

TABLE 37
36 Marker Specificity

| | Minimum | Minimum | Minimum | Minimum |
|---|---|---|---|---|
| Individual | 3 | 4 | 5 | 6 |
| marker FP | Markers | Markers | Markers | Markers |
| rate | Positive | Positive | Positive | Positive |
| 2% | 94.3% | 99.1% | 99.9% | 100.0% |
| 3% | 80.7% | 95.2% | 99.1% | 99.9% |
| 4% | | 84.9% | 96.1% | 99.2% |
| 5% | | | 88.2% | 97.0% |

TABLE 38
48 Marker Sensitivity;
Avg. Indiv. Mkr,: 50% Sensitivity

| Average | | | | | | |
|---|---|---|---|---|---|---|
| Number of | | 48 markers, | 48 markers, | 48 markers, | 48 markers, | 48 markers, |
| Molecules | Mutation, 1 | Minimum 4 | Minimum 5 | Minimum 6 | Minimum 7 | Minimum 8 |
| in Blood | Positive | Positive | Positive | Positive | Positive | Positive |
| 150 | 22.1% | 84.9% | 71.6% | 55.6% | 39.6% | 25.8% |
| 200 | 28.1% | 95.8% | 90.1% | 80.9% | 68.7% | 54.8% |
| 240 | 33.0% | 99.1% | 97.2% | 93.4% | 87.1% | 78.1% |
| 300 | 39.4% | 99.8% | 99.3% | 98.1% | 95.6% | 92.3% |
| 400 | 48.8% | 99.9% | 99.9% | 99.8% | 99.7% | 99.1% |
| 480 | 55.1% | 99.9% | 99.9% | 99.9% | 99.9% | 99.9% |
| 600 | 63.2% | 99.9% | 99.9% | 99.9% | 99.9% | 99.9% |

TABLE 39
48 Marker Sensitivity;
Avg. Indiv. Mkr,: 65% Sensitivity

| Average | | | | | | |
|---|---|---|---|---|---|---|
| Number of | | 48 markers, | 48 markers, | 48 markers, | 48 markers, | 48 markers, |
| Molecules | Mutation, 1 | Minimum 4 | Minimum 5 | Minimum 6 | Minimum 7 | Minimum 8 |
| in Blood | Positive | Positive | Positive | Positive | Positive | Positive |
| 150 | 22.1% | 95.8% | 90.0% | 80.9% | 68.7% | 54.7% |
| 200 | 28.1% | 99.3% | 98.0% | 95.2% | 90.3% | 82.9% |
| 240 | 33.0% | 99.9% | 99.6% | 98.8% | 97.1% | 94.0% |
| 300 | 39.4% | 100.0% | 100.0% | 99.9% | 99.6% | 99.0% |
| 400 | 48.8% | 100.0% | 100.0% | 100.0% | 100.0% | 100.0% |
| 480 | 55.1% | 100.0% | 100.0% | 100.0% | 100.0% | 100.0% |
| 600 | 63.2% | 100.0% | 100.0% | 100.0% | 100.0% | 100.0% |

TABLE 40
48 Marker Specificity

| | Minimum | Minimum | Minimum | Minimum | Minimum |
|---|---|---|---|---|---|
| Individual | 4 | 5 | 6 | 7 | 8 |
| marker FP | Markers | Markers | Markers | Markers | Markers |
| rate | Positive | Positive | Positive | Positive | Positive |
| 2% | 96.9% | 99.4% | 99.9% | 99.9% | 99.9% |
| 3% | 84.3% | 95.8% | 99.1% | 99.8% | 99.9% |
| 4% | | 82.5% | 95.0% | 98.8% | 99.8% |
| 5% | | | | 94.3% | 98.6% |

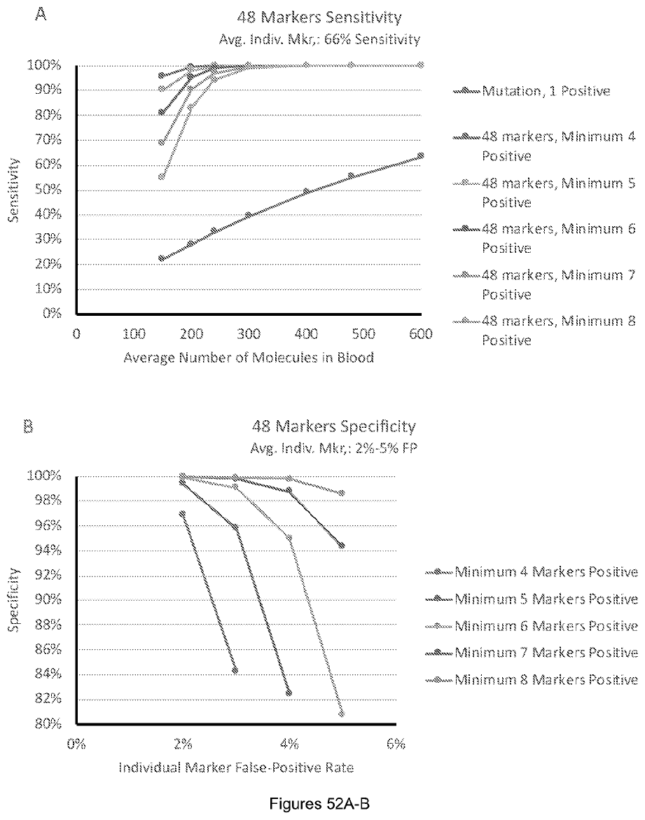

The above tables, and FIGS. 51-53, as well as FIGS. 33-35, allow for a direct comparison in the overall improvement in sensitivity when the average individual marker sensitivity improves from 50% to 66%. In this example, if there is an average of 150 molecules in the blood for the earliest cancer (Stage I), and if that would cover at least one mutation, then the sensitivity for identifying such a cancer by next generation sequencing would be 22.1% (See any of the aforementioned figures). For 24 markers, with a minimum of 3 markers positive and a 3% FP rate, overall sensitivity improves from 57.7% to 76.2%, when the average individual marker sensitivity improves from 50% to 66%, for detecting Stage I cancer (at about 150 molecules of each positive marker in the blood, see FIGS. 33A and 51A, orange line). If the individual marker FP rate is 3%, then if there is a 3-marker minimum, then overall FP rate is 5.4% for 24 markers, for a specificity of 94.6% (See FIG. 33B or 51B). However, if the individual marker FP rate is 5%, then if there is a 4-marker minimum, then overall FP rate is 6.6% for 24 markers, for a specificity of 93.4% (See FIG. 33B). At 4 markers, for Stage I cancer (at about 150 molecules of each positive marker in the blood), overall sensitivity improves from 35.3% to 56.7%, when the average individual marker sensitivity improves from 50% to 66% (See FIG. 33A and FIG. 50A). For 36 markers, with a minimum of 3 markers positive and a 2% FP rate, overall sensitivity improves from 82.6% to 93.8%, when the average individual marker sensitivity improves from 50% to 66%, for detecting Stage I cancer (at about 150 molecules of each positive marker in the blood, see FIGS. 34A and 52A). If the individual marker FP rate is 2%, then if there is a 3-marker minimum, then overall FP rate is 5.7% for 36 markers, for a specificity of 94.3% (See FIG. 34B or 52B). However, if the individual marker FP rate is 3%, then the assay requires a 4-marker minimum, then overall FP rate is 4.8% for 36 markers, for a specificity of 95.2% (See FIG. 34B). At 4 markers, for Stage I cancer (at about 150 molecules of each positive marker in the blood), overall sensitivity improves from 65.8% to 84.9%, when the average individual marker sensitivity improves from 50% to 66% (See FIG. 34A and FIG. 51A). For 48 markers, with a minimum of 4 markers positive and a 2% FP rate, overall sensitivity improves from 84.9% to 95.8%, when the average individual marker sensitivity improves from 50% to 66%, for detecting Stage I cancer (at about 150 molecules of each positive marker in the blood, see FIGS. 35A and 52A). If the individual marker FP rate is 2%, then if there is a 4-marker minimum, then overall FP rate is 3.1% for 48 markers, for a specificity of 96.9% (See FIG. 35B or 52B). However, if the individual marker FP rate is 3%, then the assay requires a 5-marker minimum, then overall FP rate is 4.2% for 48 markers, for a specificity of 95.8% (See FIG. 35B). At 5 markers, for Stage I cancer (at about 150 molecules of each positive marker in the blood), overall sensitivity improves from 71.6% to 90.0%, when the average individual marker sensitivity improves from 50% to 66% (See FIG. 35A and FIG. 52A).

From the above charts, the receiver operating characteristic (ROC) curves may be calculated by plotting Sensitivity vs. 1-Specificity. Since these are theoretical calculations, the curves were generated for different levels of average marker false-positive rates of 2%, 3%, 4%, and 5%. The AUC values, calculated for ROC curves for 24 markers, with average individual marker at 66% Sensitivity with 2%-3% FP; 36 markers, with average individual marker at 66% Sensitivity with 2%-3% FP; and 48 markers, with average individual marker at 66% Sensitivity with 2%-3% FP; are provided in the Table below. Using the benchmark of an average of 150 molecules in the blood for the earliest cancer (Stage I), and looking only at the 3% individual marker FP rate AUC values are at 77% with 24 markers (average individual marker at 50% Sensitivity), improve to 87% with 24 markers (average individual marker at 66% Sensitivity); AUC values are at 87% with 36 markers (average individual marker at 50% Sensitivity), improve to 95% with 36 markers (average individual marker at 66% Sensitivity); and AUC values are at 89% with 48 markers (average individual marker at 50% Sensitivity), improve to 97% with 48 markers (average individual marker at 66% Sensitivity). These results illustrate that for multiple marker assays achieving good sensitivities and specificities for the earliest cancers is aided when the average individual marker sensitivity improves from 50% to 66%.

TABLE 41
24, 36, & 48 Marker AUC Values from ROC Curves;
Avg. Indiv. Mkr,: 66% Sensitivity

| Total | | | | | | | |
|---|---|---|---|---|---|---|---|
| Markers: | | | | | | | |
| Individual | | | | | | | |
| marker | 150 | 200 | 240 | 300 | 400 | 480 | 600 |
| FP rate | Molecules | Molecules | Molecules | Molecules | Molecules | Molecules | Molecules |
| 24 Mkrs: 2% | 88% | 95% | 98% | >99% | >99% | >99% | >99% |
| 24 Mkrs: 3% | 87% | 94% | 97% | 99% | >99% | >99% | >99% |
| 36 Mkrs: 2% | 96% | 99% | >99% | >99% | >99% | >99% | >99% |
| 36 Mkrs: 3% | 95% | 99% | >99% | >99% | >99% | >99% | >99% |
| 48 Mkrs: 2% | 98% | >99% | >99% | >99% | >99% | >99% | >99% |
| 48 Mkrs: 3% | 97% | 99% | >99% | >99% | >99% | >99% | >99% |

How would increasing the average individual marker sensitivity from 50% sensitivity to 66% sensitivity improve upon a one-step cancer assay? To review: the challenge is to screen 107 million adults in the U.S. over the age of 50 for colorectal cancer—of which there are about 135,000 new cases that are diagnosed a year. In this example, if there is an average of 300 molecules in the blood for early cancer (Stage I & II), and taking the best-case scenario of individual marker FP rate is 2%, then if there is a 3-marker minimum, then overall FP rate is 1.6% for 24 markers, for a specificity of 98.4% (See FIG. 33B or 50B). At 3 markers, for Stage I & II cancer (at about 300 molecules of each positive marker in the blood), for average marker sensitivity of 50%, the test would miss 6.2%; i.e. for Stage I & II cancer the overall sensitivity would be 93.8% (See FIG. 33A), e.g. the test would correctly identify 93.8% of individuals with disease, which would be 126,630 individuals (out of 135,000 new cases). At a specificity of 98.4%, for 107 million individuals screened, the test would also generate 1.6%×107,000,000=1,712,000 false positives. Thus, the positive predictive value would be 126,630/(126,630+1,712,000)=around 6.8%, in other words, only one in 14 individuals who tested positive would actually have colorectal cancer, the rest would be false-positives. At 3 markers, for Stage I & II cancer (at about 300 molecules of each positive marker in the blood), for average marker sensitivity of 66%, the test would miss 1.4%; i.e. for Stage I & II cancer the overall sensitivity would be 98.6% (See FIG. 50A), e.g. the test would correctly identify 98.6% of individuals with disease, which would be 133,110 individuals (out of 135,000 new cases). At a specificity of 98.4%, for 107 million individuals screened, the test would also generate 1.6%×107,000,000=1,712,000 false positives. Thus, the positive predictive value would be 133,110/(133,110+1,712,000)=around 7.2%, in other words, only one in 14 individuals who tested positive would actually have colorectal cancer, the rest would be false-positives. Thus, if the FP is low, i.e. 2%, then there is marginal benefit in going from an average marker sensitivity of 50% to an average marker sensitivity of 66%.

However, if the individual marker FP rate is more realistic, say 4%, then more markers will be required to achieve better than 98% specificity, and this will be at the cost of sensitivity. If individual marker FP rate is 4%, then if there is a 5-marker minimum, then overall FP rate is 0.4% for 24 markers, for a specificity of 99.6% (See FIG. 33B). At 5 markers, for Stage I & II cancer (at about 300 molecules of each positive marker in the blood), at an average marker sensitivity of 50%, the test would miss 28.5%; i.e. for Stage I & II cancer the overall sensitivity would be 71.5% (See FIG. 33A), e.g. the test would correctly identify 71.5% of individuals with disease, which would be 90,540 individuals (out of 135,000 new cases). At a specificity of 99.6%, for 107 million individuals screened, the test would also generate 0.4%×107,000,000=428,000 false positives. Thus, the positive predictive value would be 90,540/(90,540+428,000)=around 17.5%, in other words, one in 5.7 individuals who tested positive would actually have colorectal cancer, the rest would be false-positives. A PPV of 17.5% is quite respectable, however, it would be achieved at the cost of missing 28.5% of early cancer. At 3 markers, for Stage I & II cancer (at about 300 molecules of each positive marker in the blood), for average marker sensitivity of 66%, the test would miss 10.0%; i.e. for Stage I & II cancer the overall sensitivity would be 90.0% (See FIG. 50A), e.g. the test would correctly identify 90.0% of individuals with disease, which would be 121,500 individuals (out of 135,000 new cases). At a specificity of 99.6%, for 107 million individuals screened, the test would also generate 0.4%×107,000,000=428,000 false positives. Thus, the positive predictive value would be 121,500/(121,500+428,000)=around 22.1%, in other words, one in 4.5 individuals who tested positive would actually have colorectal cancer, the rest would be false-positives. A PPV of 22.1% is excellent, and further, it would be achieved at the cost of missing only 10% of early cancer. Thus, if the FP is more realistic i.e. 4%, then there is a significant benefit in going from an average marker sensitivity of 50% to an average marker sensitivity of 66%.

Returning to the example of colorectal cancer, in particular the cases of microsatellite stable tumors (MSS) where the mutation load is low, for these calculations when relying on NGS sequencing alone (assuming 150 molecules with one mutation in the blood), an estimated 78% of early colorectal cancer would be missed. Again, to put these number in perspective, in the U.S., about 135,000 new cases of colorectal cancer were diagnosed in 2018, of which about 60% is late cancer (i.e. Stage III & IV). About 107 million individuals in the U.S. are over the age of 50 and should be tested for colorectal cancer. While it cannot be predicted how many individuals have a hidden cancer (i.e. Stage I) within them, who are non-compliant to testing, for the purposes of this calculation, assume that the average late cancer was once the average early cancer, and thus individuals with Stage I cancer would be about 40,500 individuals. With the assumption of these samples containing at least 150 molecules with one mutation in the blood, such a test would find 8,910 individuals (out of 40,500 individuals with Stage I cancer) with colorectal cancer. However, with a specificity for sequencing at 98%, there would be about 2.1 million false-positives. The positive predictive value of such a test would be around 0.4%, in other words, only one in 236 individuals who tested positive would actually have Stage I colorectal cancer, the rest would be false-positives. In contrast, consider the two-step methylation marker test described above, wherein the first step has 24 methylation markers specific to GI cancers, while the second step has 48 methylation markers specific to colorectal cancer. In this example, the average individual marker sensitivity is set at 66%. In this example, as above, the calculations are done with the anticipation of an average of 150 methylated molecules per positive marker in the blood. Assuming individual marker false-positive rates of 3%, and with the first step requiring a minimum of 3 markers positive, then with an overall specificity of 94.6%, the first step would identify 5,778,000 individuals (out of 107,000,000 total adults over 50 in the U.S.) which would include at 76.2% sensitivity or about 30,861 individuals with Stage I colorectal cancer (out of 40,500 individuals with Stage I cancer). However, those 5,778,000 presumptive positive individuals would be evaluated in the second step of 48 markers requiring a minimum of 5 markers positive, then the two-step test would identify 76.2%×90.0%=68.6%=27,775 individuals (out of 40,500 individuals with Stage I cancer) with colorectal cancer. With a specificity of 95.8%, the second test would also generate 5,778,000×4.2%=242,676 false-positives. The positive predictive value of such a test would be 27,775/270,451=10.3%, in other words, 1 in 10 individuals who tested positive would actually have Stage I colorectal cancer, an extraordinarily successful screen to focus on those patients who would most benefit from follow-up colonoscopy. Since >90% of individuals identified with Stage I colon cancer have long-term survival after just surgery, the benefit in lives saved would be of incalculable value.

How would the above numbers change if the initial test in the two-step assay uses 36 markers? In this example, as above, the calculations are done with the anticipation of an average of 150 methylated molecules per positive marker in the blood. Assuming individual marker false-positive rates of 3%, and with the first step requiring a minimum of 4 markers positive, then with an overall specificity of 95.2%, the first step would identify 5,136,000 individuals (out of 107,000,000 total adults over 50 in the U.S.) which would include at 84.9% sensitivity or about 34,385 individuals with Stage I colorectal cancer (out of 40,500 individuals with Stage I cancer). However, those 5,136,000 presumptive positive individuals would be evaluated in the second step of 48 markers requiring a minimum of 5 markers positive, then the two-step test would identify 84.9%×90.0%=76.4%=30,946 individuals (out of 40,500 individuals with Stage I cancer) with colorectal cancer. With a specificity of 95.8%, the second test would also generate 5,136,000×4.2%=215,712 false-positives. The positive predictive value of such a test would be 30,946/246,658=12.5%, in other words, 1 in 8 individuals who tested positive would actually have Stage I colorectal cancer. In reality, one would need to also include the success for identifying Stage 2 and higher cancers. In expanding this example, the calculations are done with the anticipation that Stage I CRC has an average of 150 methylated molecules per positive marker in the blood, Stage II CRC has an average of 200 methylated molecules per positive marker, and the higher stages (III & IV) have at least an average of 300 methylated molecules per positive marker, and the higher stages. Also, to be consistent with the idea that as the test is used repeatedly, more of early and less of late CRC will be detected, then an estimate of 40,500 individuals with Stage I cancer, 40,500 individuals with Stage II cancer, and the remaining 54,000 individuals have late-stage cancer=135,000 total individuals with colorectal cancer identified per year in the U.S. The above calculation already provided the false-positive rate for the early cancer. For Stage II cancer, 95.8% would be identified in the first step, of which 95.8%×98.0%=93.9%=38,023 individuals with Stage II cancer would be verified in the second step. For Stage III and IV cancer, 99.8% would be identified in the first step, of which 99.8%×(100%)=53,892 individuals with late cancer would be identified. This brings the total identified at 30,946+38,023+53,892=122,861 individuals out of 135,000 with colorectal cancer. Overall, the positive predictive value of such a test would be 122,861/369,519=33.2%, in other words, 1 in 3 individuals who tested positive would actually have colorectal cancer, and this test would identify 68,969/81,000 or 85% of those individuals with early cancer—which would be unprecedented in diagnostic approaches to detect early cancer.

The ultimate goal is to develop a high-throughput scalable test to detect the majority of cancers that occur worldwide. The solid tumor cancers have been grouped into the following subclasses, as listed below in Tables 42, 43, and 44 for both sexes, for men, and for women.

TABLE 42
Global cancer incidence; Both Sexes
(Numbers in thousands; most common cancers
have incidence above 100,000 per year)

| | Incidence | % Group | % total All | (Total) |
|---|---|---|---|---|
| Group 1: | | | | |
| Colorectal (1,801) | 1801 | 52.9% | 12.9% | 13981 |
| Stomach (1,033) | 1033 | 30.3% | 7.4% | 13981 |
| Esophagus (572) | 572 | 16.8% | 4.1% | 13981 |
| Total, Group 1: | 3406 | | | |
| Group 2: | | | | |
| Breast (2,089) | 2089 | 62.6% | 14.9% | 13981 |
| Endometrial & Cervical | 570 | 17.1% | 4.1% | 13981 |
| (570) | | | | |
| Uterine (382) | 382 | 11.5% | 2.7% | 13981 |
| Ovarian (295) | 295 | 8.8% | 2.1% | 13981 |
| Total, Group 2: | 3336 | | | |
| Group 3: | | | | |
| Lung (2,093) | 2093 | 59.9% | 15.0% | 13981 |
| Head & Neck (832) | 832 | 23.8% | 6.0% | 13981 |
| Thyroid (567) | 567 | 16.2% | 4.1% | 13981 |
| Total, Group 3: | 3492 | | | |
| Group 4: | | | | |
| Prostate (1,276) | 1276 | 57.3% | 9.1% | 13981 |
| Bladder (549) | 549 | 24.6% | 3.9% | 13981 |
| Kidney (403) | 403 | 18.1% | 2.9% | 13981 |
| Total, Group 4: | 2228 | | | |
| Group 5: | | | | |
| Liver (841) | 841 | 55.4% | 6.0% | 13981 |
| Pancreas (459) | 459 | 30.2% | 3.3% | 13981 |
| Gallbladder (219) | 219 | 14.4% | 1.6% | 13981 |
| Total, Group 5: | 1519 | | | |
| Total | 13981 | | | |

TABLE 43
Global cancer incidence; Male
(Numbers in thousands; most common cancers
have incidence above 100,000 per year)

| | Incidence | % Group | % total All | (Total) |
|---|---|---|---|---|
| Group 1: | | | | |
| Colorectal (1,801) | 1006 | 48.2% | 14.1% | 7114 |
| Stomach (1,033) | 683 | 32.7% | 9.6% | 7114 |
| Esophagus (572) | 400 | 19.1% | 5.6% | 7114 |
| Total, Group 1: | 2089 | | | |
| Group 2: | | | | |
| Breast (2,089) | | | | |
| Endometrial & Cervical | | | | |
| (570) | | | | |
| Uterine (382) | | | | |
| Ovarian (295) | | | | |
| Total, Group 2: | | | | |
| Group 3: | | | | |
| Lung (2,093) | 1368 | 64.1% | 19.2% | 7114 |
| Head & Neck (832) | 635 | 29.8% | 8.9% | 7114 |
| Thyroid (567) | 131 | 6.1% | 1.8% | 7114 |
| Total, Group 3: | 2134 | | | |
| Group 4: | | | | |
| Prostate (1,276) | 1276 | 65.3% | 17.9% | 7114 |
| Bladder (549) | 424 | 21.7% | 6.0% | 7114 |
| Kidney (403) | 254 | 13.0% | 3.6% | 7114 |
| Total, Group 4: | 1954 | | | |
| Group 5: | | | | |
| Liver (841) | 597 | 63.7% | 8.4% | 7114 |
| Pancreas (459) | 243 | 25.9% | 3.4% | 7114 |
| Gallbladder (219) | 97 | 10.4% | 1.4% | 7114 |
| Total, Group 5: | 937 | | | |
| Total | 7114 | | | |

TABLE 44
Global cancer incidence; Female
(Numbers in thousands; most common cancers
have incidence above 100,000 per year)

| | Incidence | % Group | % total All | (Total) |
|---|---|---|---|---|
| Group 1: | | | | |
| Colorectal (1,801) | 795 | 60.4% | 11.5% | 6930 |
| Stomach (1,033) | 350 | 26.6% | 5.1% | 6930 |
| Esophagus (572) | 172 | 13.1% | 2.5% | 6930 |
| Total, Group 1: | 1317 | | | |
| Group 2: | | | | |
| Breast (2,089) | 2089 | 62.6% | 30.1% | 6930 |
| Endometrial & Cervical | 570 | 17.1% | 8.2% | 6930 |
| (570) | | | | |
| Uterine (382) | 382 | 11.5% | 5.5% | 6930 |
| Ovarian (295) | 295 | 8.8% | 4.3% | 6930 |
| Total, Group 2: | 3336 | | | |
| Group 3: | | | | |
| Lung (2,093) | 725 | 53.4% | 10.5% | 6930 |
| Head & Neck (832) | 196 | 14.4% | 2.8% | 6930 |
| Thyroid (567) | 436 | 32.1% | 6.3% | 6930 |
| Total, Group 3: | 1357 | | | |
| Group 4: | | | | |
| Prostate (1,276) | 0 | 0.0% | 0.0% | 6930 |
| Bladder (549) | 216 | 63.9% | 3.1% | 6930 |
| Kidney (403) | 122 | 36.1% | 1.8% | 6930 |
| Total, Group 4: | 338 | | | |
| Group 5: | | | | |
| Liver (841) | 244 | 41.9% | 3.5% | 6930 |
| Pancreas (459) | 216 | 37.1% | 3.1% | 6930 |
| Gallbladder (219) | 122 | 21.0% | 1.8% | 6930 |
| Total, Group 5: | 582 | | | |
| Total | 6930 | | | |

The above list does not include liquid cancers, nor some of the less common solid tumors. Worldwide incidence (numbers in thousands) of liquid tumors include Non-Hodgkin lymphoma (225), leukemia (187), multiple myeloma (70), and Hodgkin lymphoma (33). These would be detected in a separate test not discussed herein. Further, the list excludes melanoma (287) and brain tumors (134). Testing for these would be done with separate sets of markers, optimized as described above for colorectal cancer. In addition, while some cancers listed in the tables above are of extreme medical importance (e.g., mesothelioma, thyroid cancer, and the three different subcategories of kidney cancer), their biology is sufficiently different as to usually merit a separate set of markers, again, optimized as described above for colorectal cancer.

Thus, for the present application, a Pan-Oncology test is developed that would include the following major cancers by the following groupings: Group 1 (colorectal, stomach, and esophagus); Group 2 (breast, endometrial, ovarian, cervical, and uterine); Group 3 (lung and head & neck); Group 4 (prostate and bladder); and Group 5 (liver, pancreatic, or gall bladder). Note that some cancers within Group 3 may be tested as a sputum sample, and cancers in Group 4 may be tested as a urine sample.

Careful analysis of the TCGA methylation database revealed a general commonality in methylation patterns among cancers within these 5 separate groups. Further, there are some methylation markers that are common among several cancers, while absent in normal white blood cells. The following strategy was used to design a multi-step pan-oncology test.

The first step is to identify markers that cover multiple cancers in one or more of the above groups. The markers should be sufficiently diverse as to cover cancers in all 5 groups. For example, a first step of the assay would use a set of 96 markers that on average comprise of at least 36 markers with 50% sensitivity that covers each of the aforementioned 16 types of solid tumors (covered in the 5 Groups; see FIG. 1E; for 66% sensitivity, see FIG. 1C). If at least 5 markers are positive, the assay would then move to a second step that would be used to verify the initial results and identify the most probable tissue of origin. In most cases, more than 5 markers would be positive, and then pattern of distribution of these methylation markers would guide the choice of which groups to test in the second step. The second step of the assay would test, on average, 2 or more sets of the group-specific markers. For example, the second step of the assay would use 2 or more sets of 64 group-specific markers that, on average, comprise of at least 36 markers with 50% sensitivity that covers each of the aforementioned types of solid tumors that may be present in that group (for 66% sensitivity, see FIG. 1D). By scoring the markers that are positive and comparing to predicted positives for each cancer type within the group tested, the physician can identify the most probable tissue of origin, and subsequently send the patient to the appropriate imaging.

A close evaluation of the TCGA database reveals pan-oncology markers that meet the criteria for use in a set of 96 markers that on average comprise of at least 36 markers with 50% sensitivity that covers each of the aforementioned 16 types of solid tumors. These pan-oncology markers include but are not limited to cancer-specific microRNA markers, cancer-specific ncRNA and lncRNA markers, cancer-specific exon transcripts, tumor-associated antigens, cancer protein markers, protein markers that can be secreted by solid tumors into the blood, common mutations, primary CpG sites that are solid tumor and tissue specific markers, chromosomal regions or sub-regions within which are primary CpG sites that are solid tumor and tissue specific markers, and primary and flanking CpG sites that are solid tumor and tissue specific markers. Methods for detecting said markers have been discussed supra, and these markers are listed below and in accompanying figures.

Blood-based, solid tumor-specific microRNA markers derived through analysis of TCGA microRNA datasets, includes, but is not limited to, the following markers: (mir ID, Gene ID); hsa-mir-21, MIR21; hsa-mir-182, MIR182; hsa-mir-454, MIR454; hsa-mir-96, MIR96; hsa-mir-183, MIR183; hsa-mir-549, MIR549; hsa-mir-301^{a}, MIR301A; hsa-mir-548f-1, MIR548F1; hsa-mir-301b, MIR301B; hsa-mir-103-1, MIR1031; hsa-mir-18^{a}, MIR18A; hsa-mir-147b, MIR147B; hsa-mir-4326, MIR4326; and hsa-mir-573, MIR573 These markers may be present in exosomes, tumor-associated vesicles, Argonaute complexes, or other protected states in the blood.

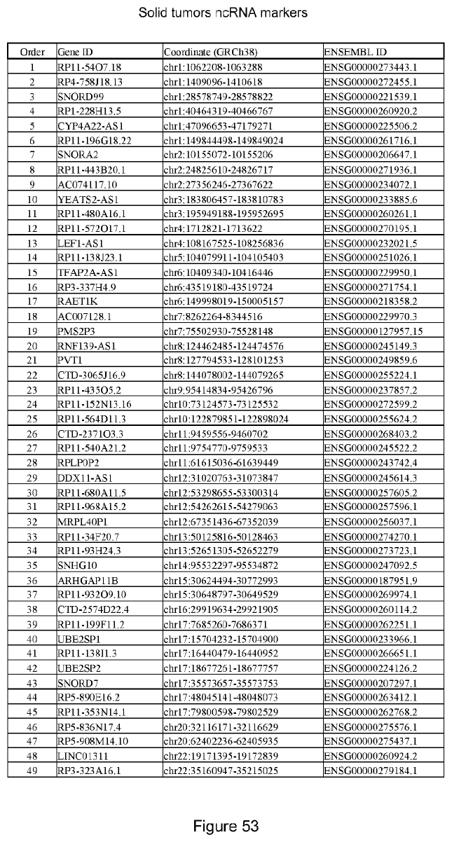

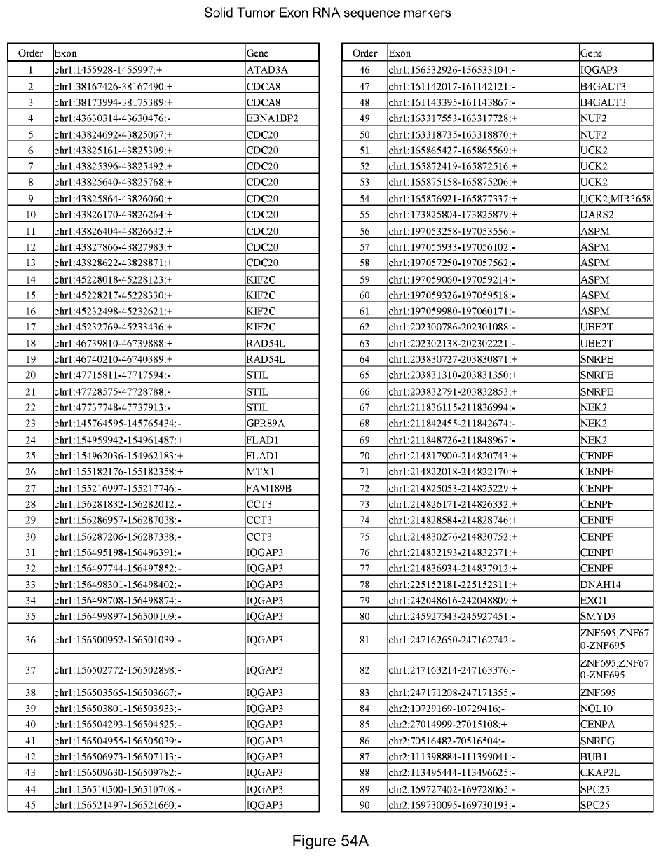

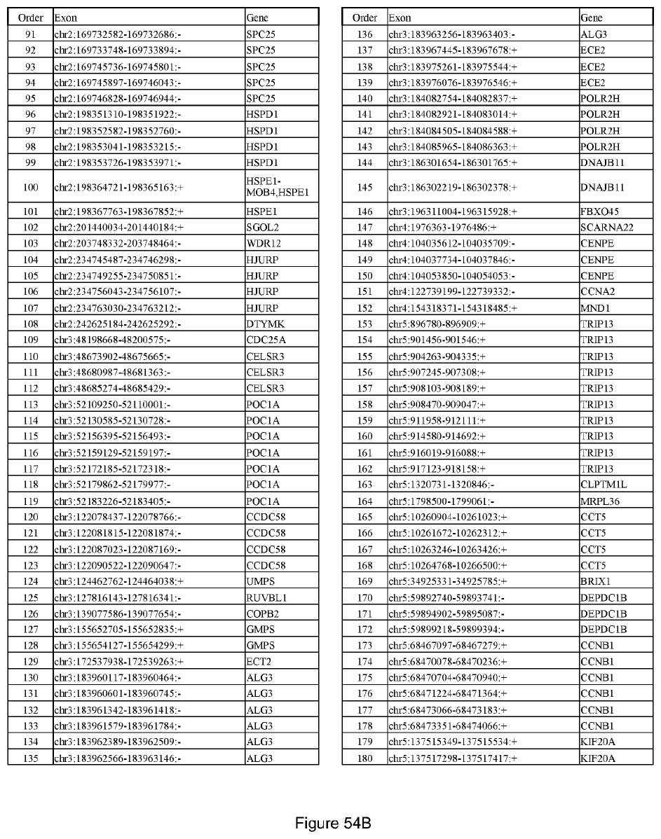

FIG. 53 provides a list of blood-based, solid tumor-specific ncRNA and lncRNA markers derived through analysis of various publicly available Affymetrix Exon ST CEL data, which were aligned to GENCODE annotations to generate ncRNA and lncRNA transcriptome datasets. Comparative analyses across these datasets (various cancer types, along with normal tissues, and peripheral blood) were conducted to generate the ncRNA and lncRNA markers list. Such lncRNA and ncRNA may be enriched in exosomes or other protected states in the blood.

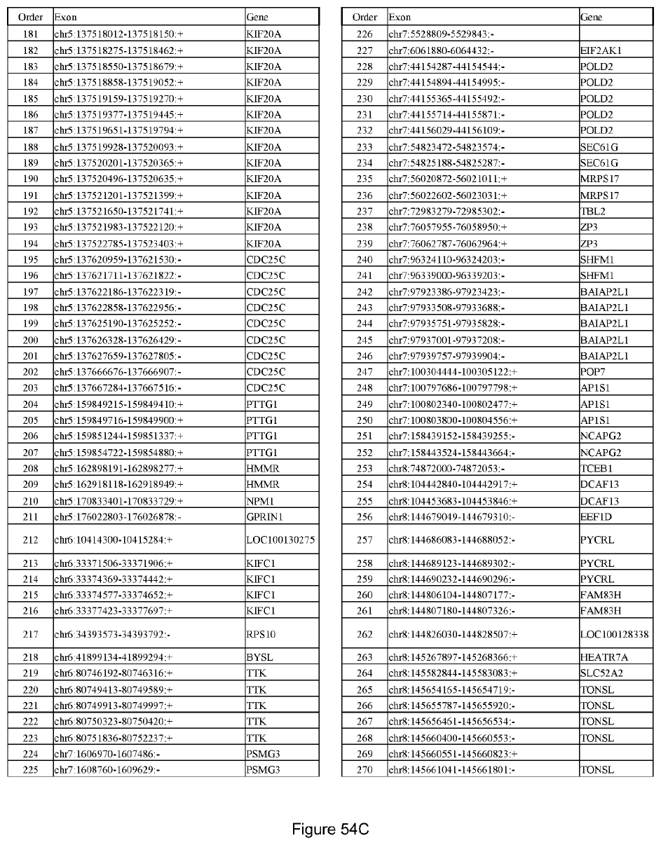

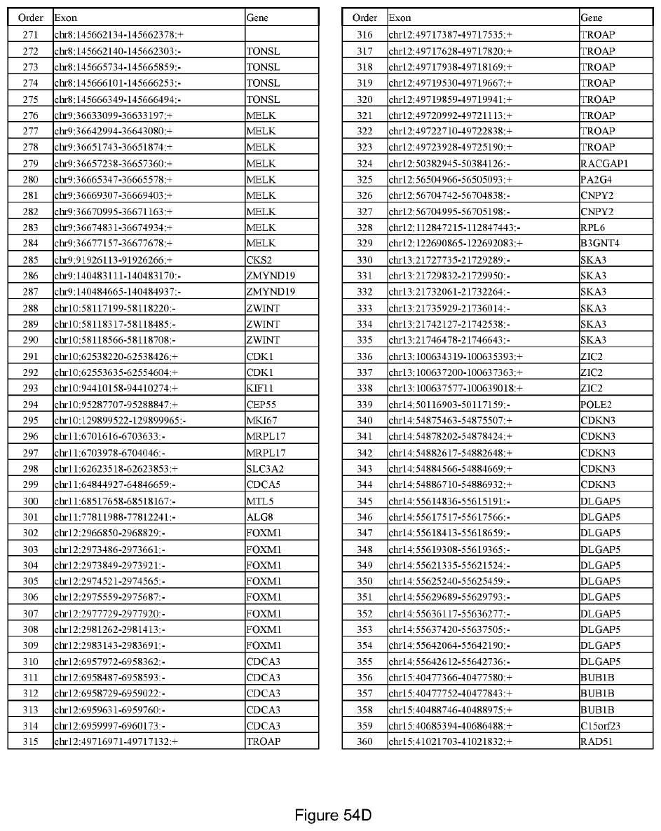

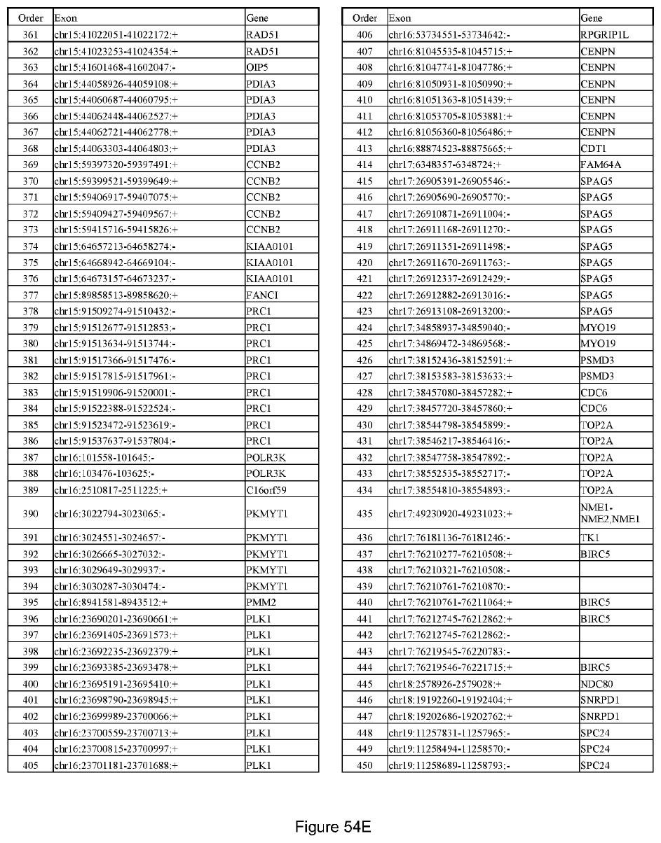

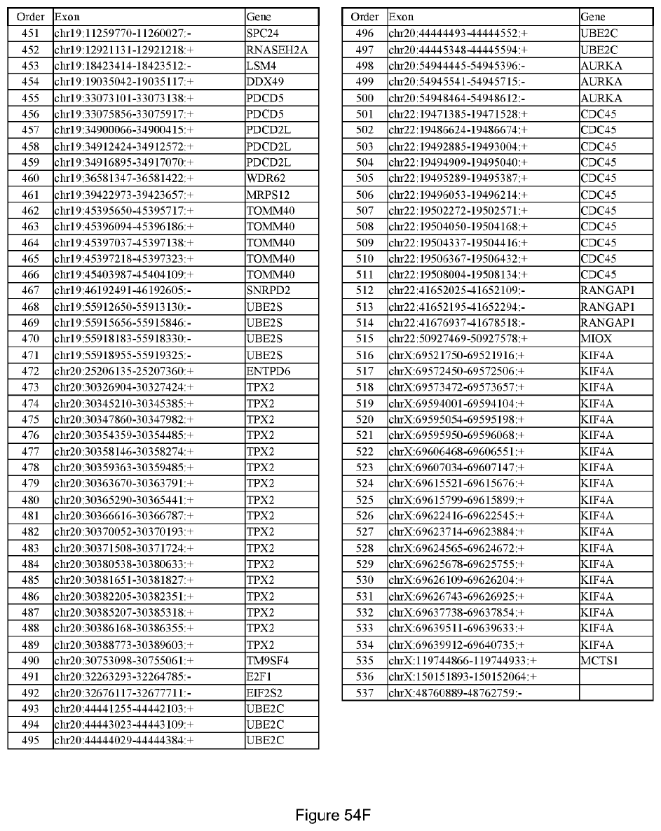

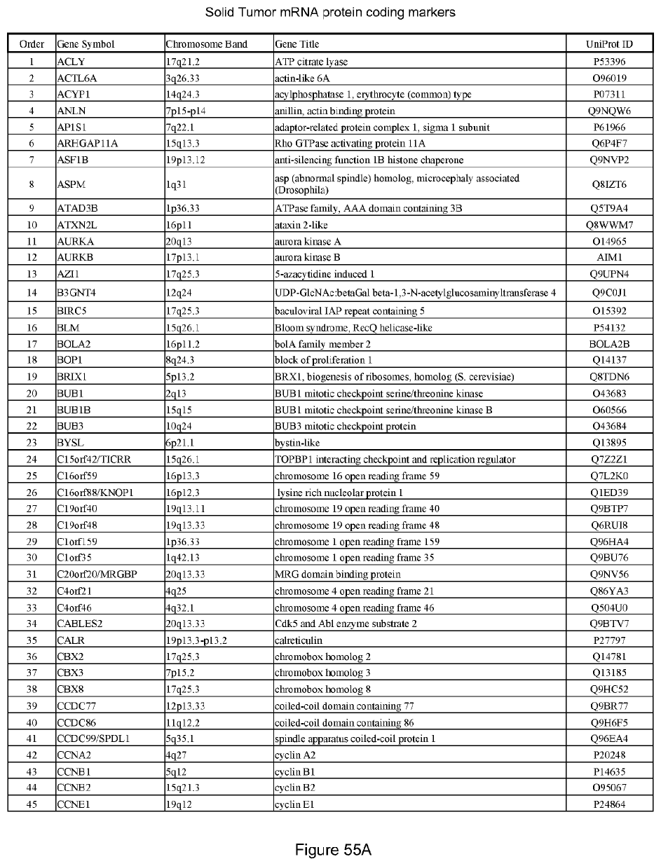

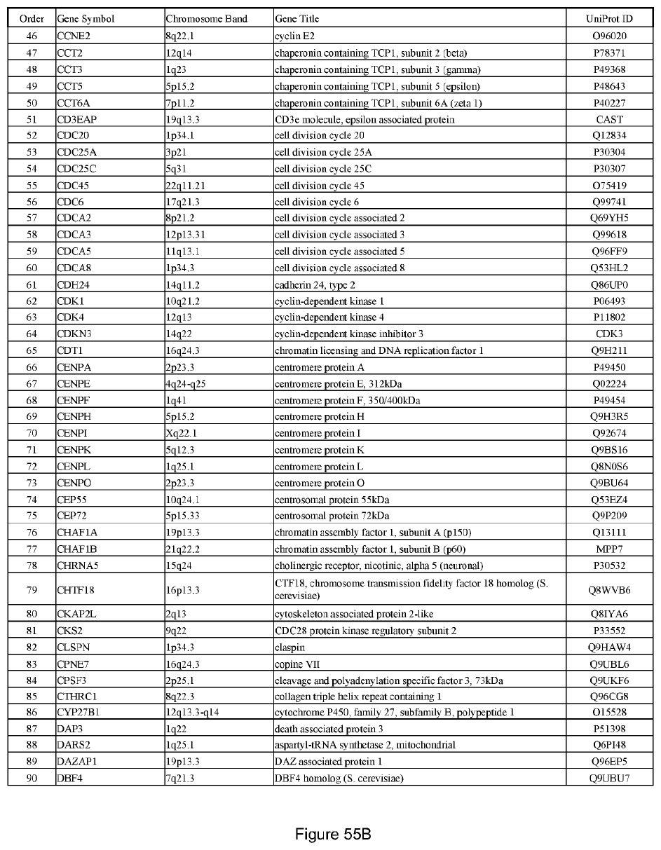

In addition, FIG. 54 provides a list of blood-based solid tumor-specific exon transcripts that may be enriched in exosomes, tumor-associated vesicles, or other protected states in the blood. Overexpressed oncogene transcripts, or transcripts of mutant oncogenes may be enriched in exosomes, as they may drive spread of the cancer.

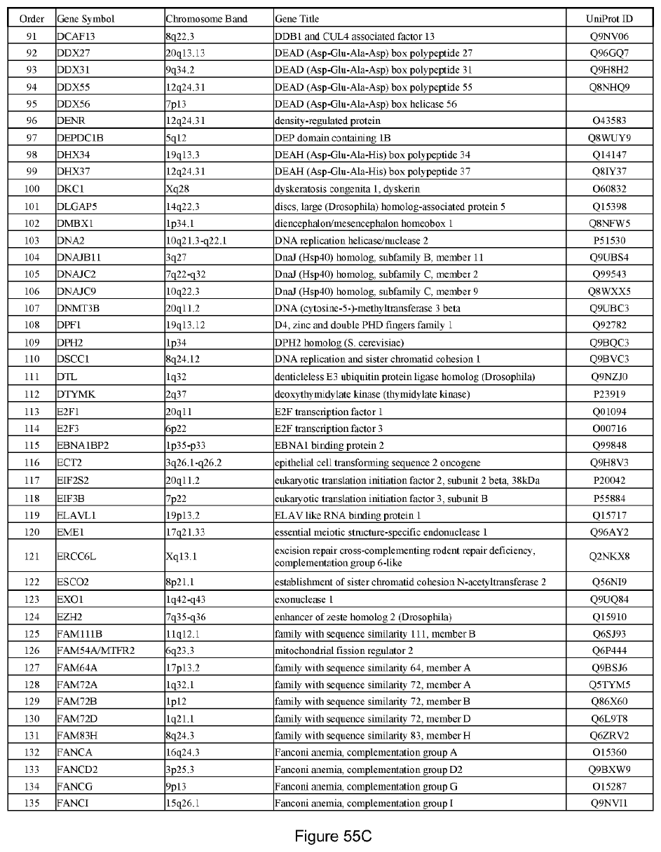

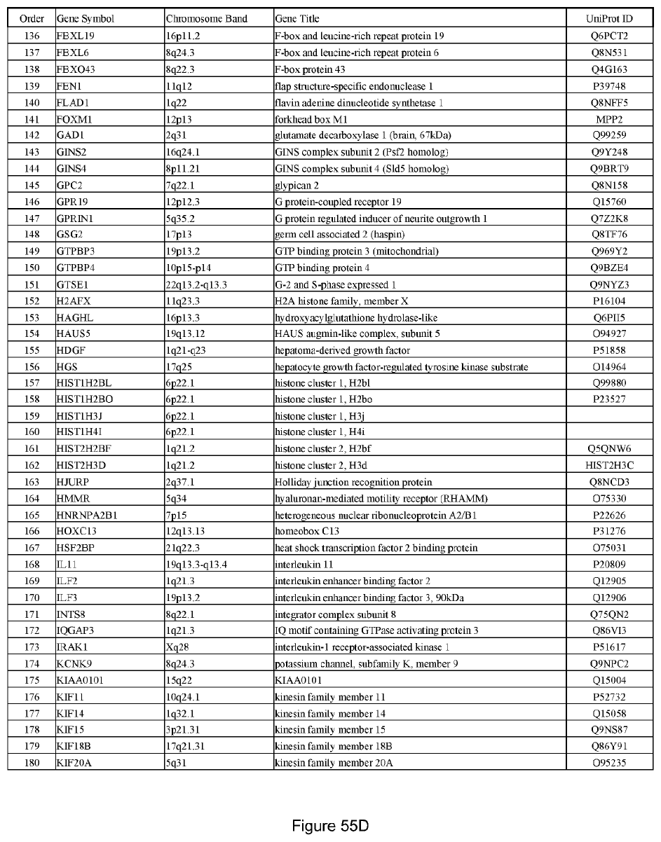

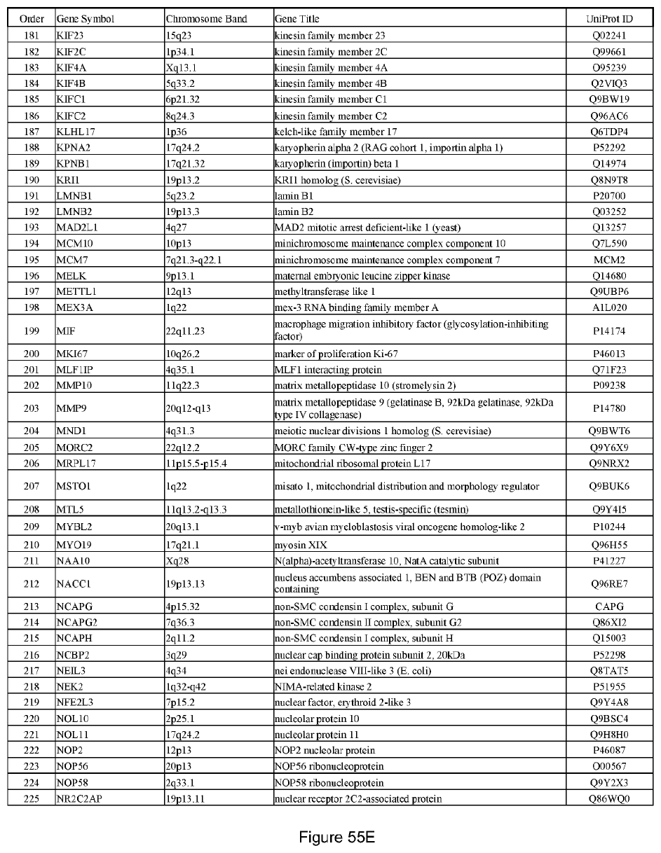

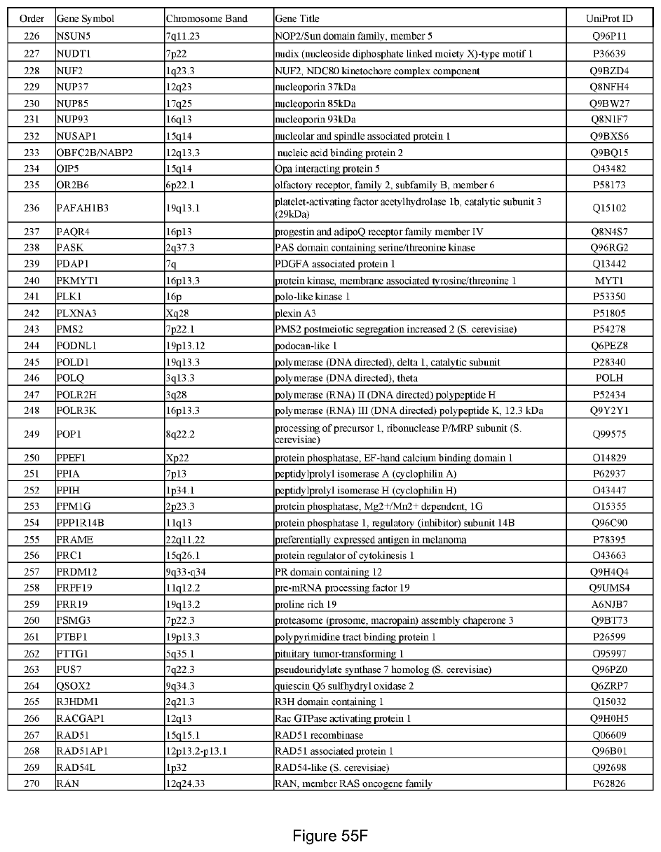

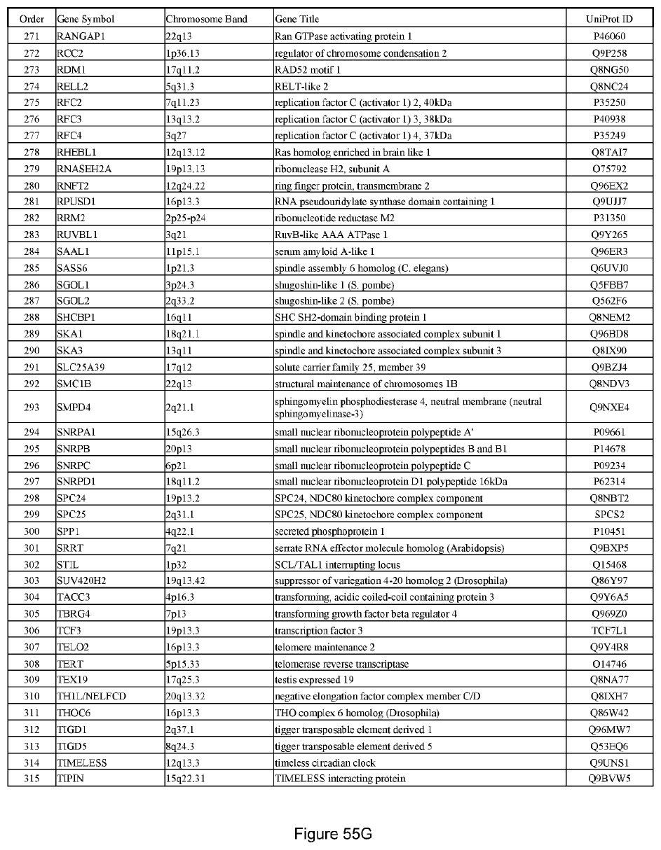

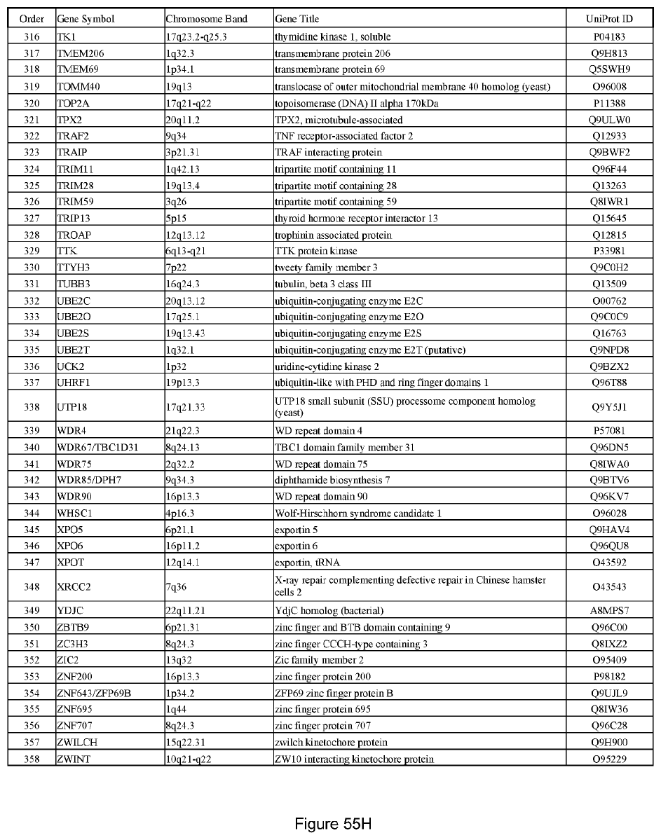

FIG. 55 provides a list of cancer protein markers, identified through mRNA sequences, protein expression levels, protein product concentrations, cytokines, or autoantibody to the protein product arising from solid tumors, which may be identified in the blood, either within exosomes, other protected states, tumor-associated vesicles, or free within the plasma.

Protein markers that can be secreted by solid tumors into the blood include, but are not limited to: (Protein name, UniProt ID); Uncharacterized protein C19orf48, Q6RUI8; Protein FAM72B, Q86X60; Protein FAM72D, Q6L9T8; Hydroxyacylglutathione hydrolase-like protein, Q6PII5; Putative methyltransferase NSUN5, Q96P11; RNA pseudouridylate synthase domain-containing protein 1, Q9UJJ7; Collagen triple helix repeat-containing protein 1, Q96CG8; Interleukin-11, P20809; Stromelysin-2, P09238; Matrix metalloproteinase-9, P14780; Podocan-like protein 1, Q6PEZ8; Putative peptide YY-2, Q9NRI6; Osteopontin, P10451; Sulfhydryl oxidase 2, Q6ZRP7; Glypican-2, Q8N158; Macrophage migration inhibitory factor, P14174; Peptidyl-prolyl cis-trans isomerase A, P62937; Calreticulin, P27797. A comparative analysis was performed across various TCGA datasets (tumors, normals), followed by an additional bioinformatics filter (Meinken et al., “Computational Prediction of Protein Subcellular Locations in Eukaryotes: an Experience Report,” Computational Molecular Biology 2(1):1-7 (2012), which is hereby incorporated by reference in its entirety), which predicts the likelihood that the translated protein is secreted by the cells.

The distribution of mutations in solid tumors are available in the public COSMIC database, with the commonly altered genes across solid tumors listed as: TP53 (tumor protein p53), TTN (titin), MUC16 (mucin 16), and KRAS (Ki-ras2 Kirsten rat sarcoma viral oncogene homolog).

A deep analysis of the TCGA database of methylation markers that are absent in blood but on average are present in solid tumor types at 50% sensitivity show three general categories of clusters: (i) Markers that are present in colorectal cancers, and related GI cancer (stomach & esophagus), (ii) Markers that are present in colorectal cancers, and related GI cancer (stomach & esophagus), as well as other tumors, and (iii) Markers that are mostly absent in colorectal cancers, but present in other tumors. Second, while for some tumor types one could readily identify markers that were unique to that group, such as Group 2 (breast, endometrial, ovarian, cervical, and uterine), for other tumor types such as lung cancer or pancreatic cancer, it was difficult to identify methylation markers that were unique to that cancer. Consequently, to assemble a set of 96 markers that satisfied the criteria of at least 36 markers with 50% sensitivity that covers each of the aforementioned 16 types of solid tumors, the first 48 markers comprised of about 12 markers that were strongly represented in Group 2 tumors, about 12 markers that were strongly represented in Group 3 tumors, about 12 markers that were strongly represented in Group 4 tumors, and about 12 markers that were strongly represented in Group 5 tumors. The remaining 48 markers comprised of about 12 markers that were strongly represented in Groups 1 & 2 tumors, about 12 markers that were strongly represented in Groups 1 & 3 tumors, about 12 markers that were strongly represented in Groups 1 & 4 tumors, and about 12 markers that were strongly represented in Groups 1 & 5 tumors.

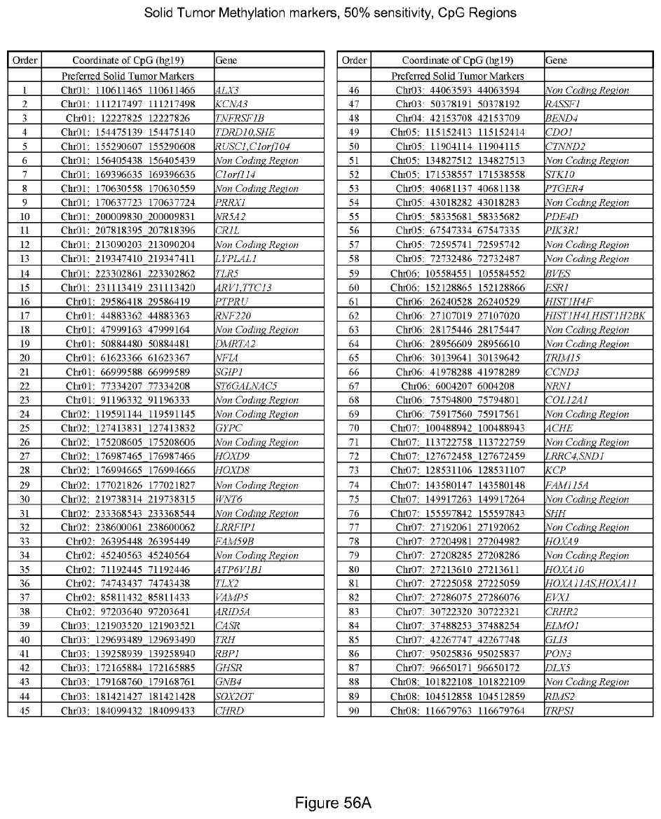

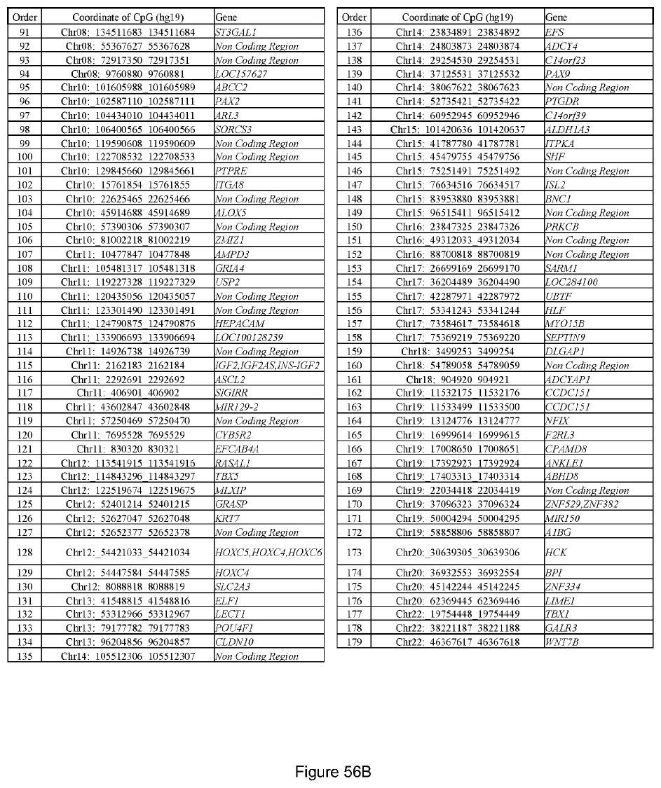

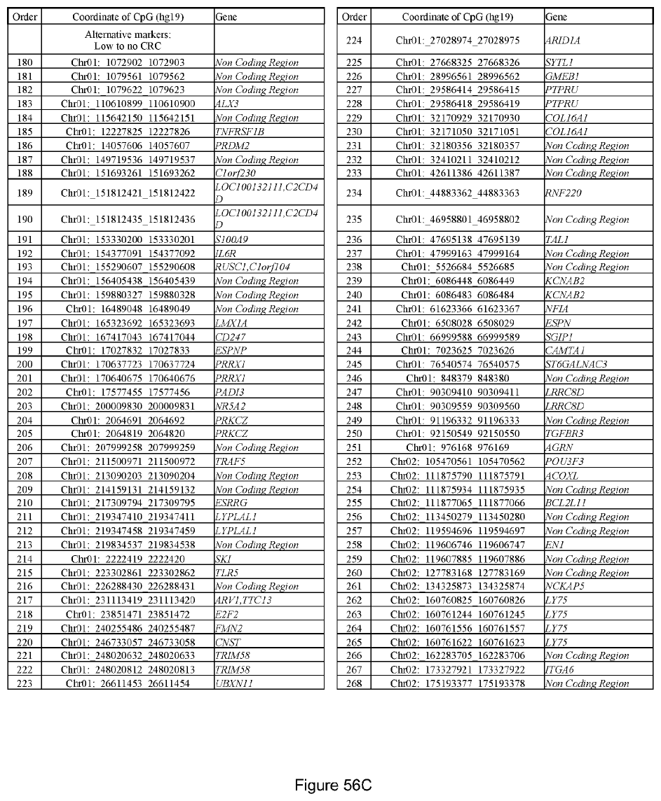

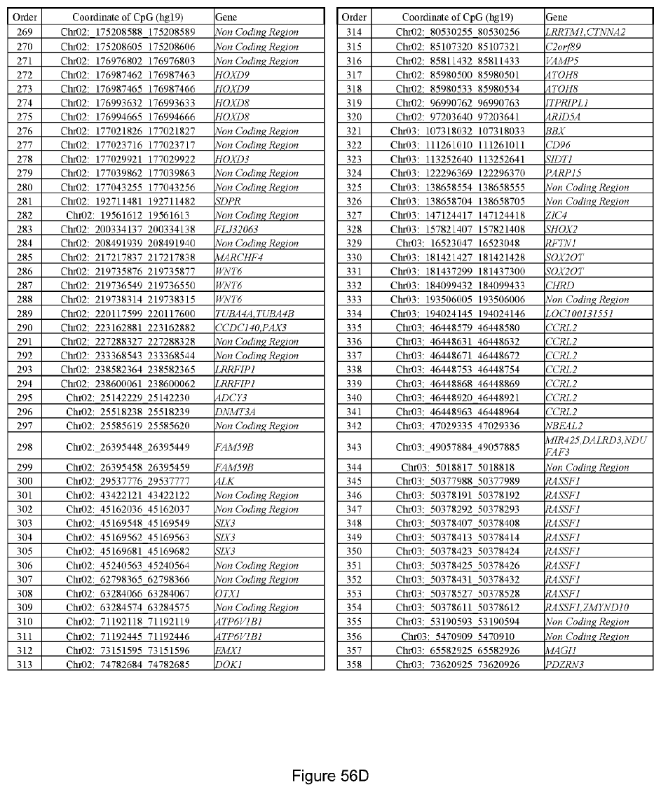

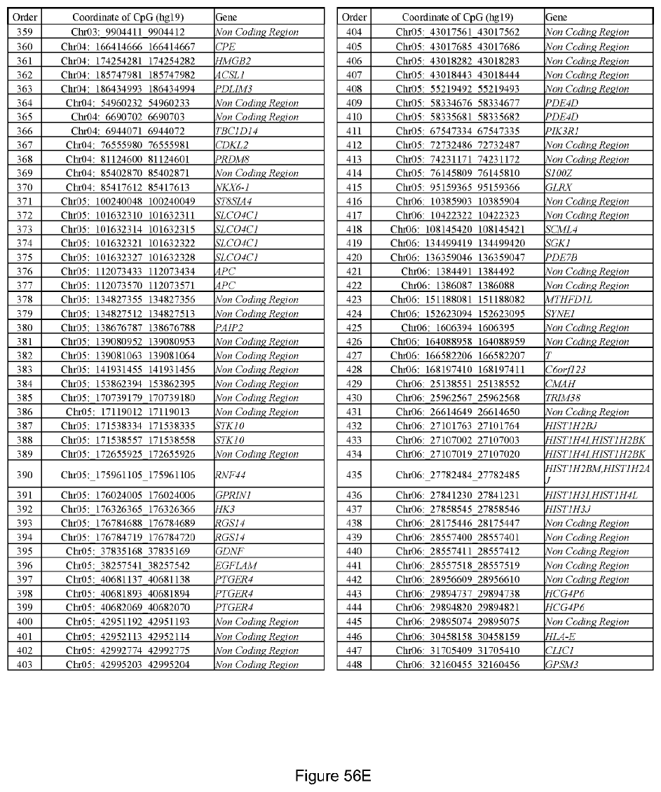

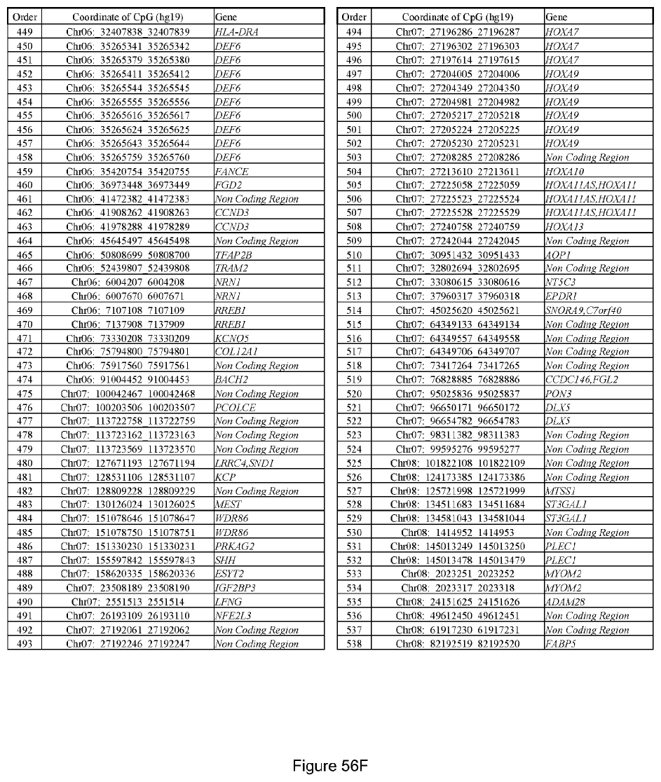

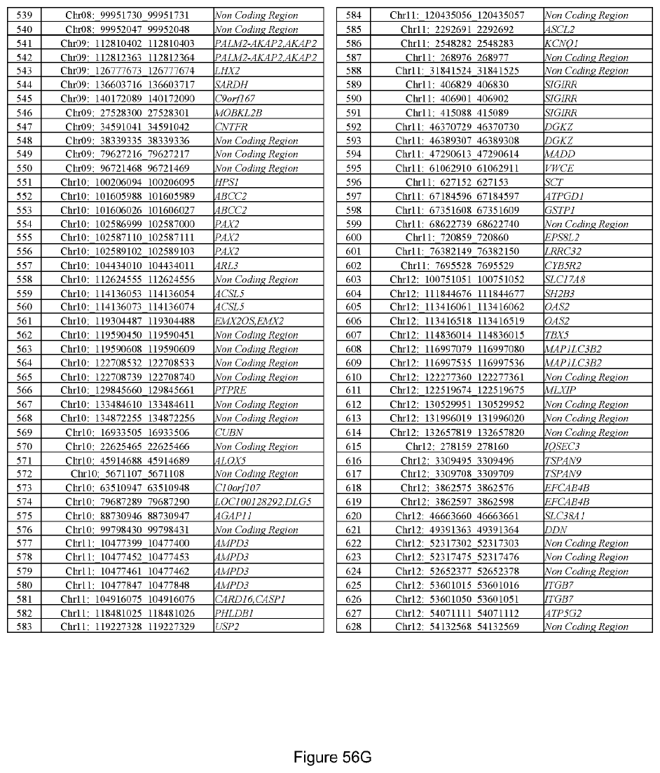

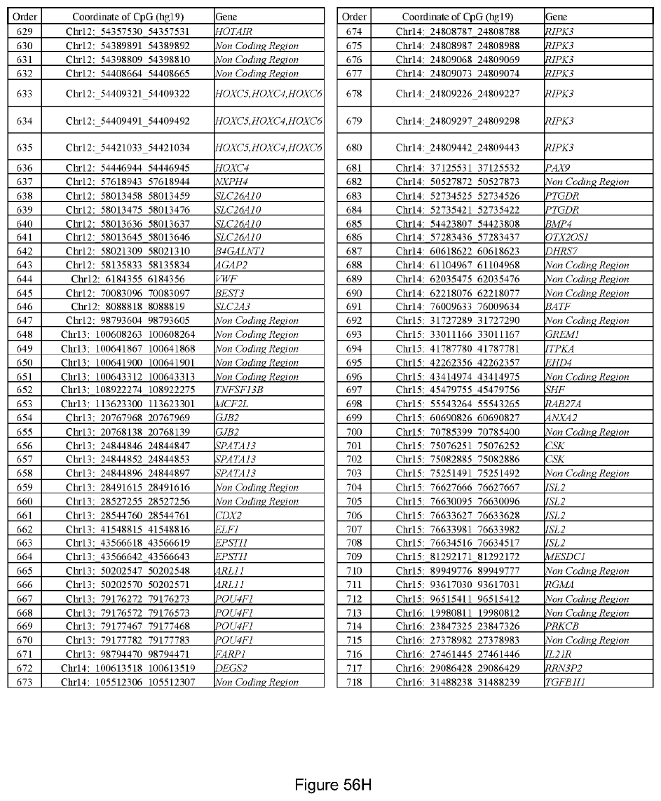

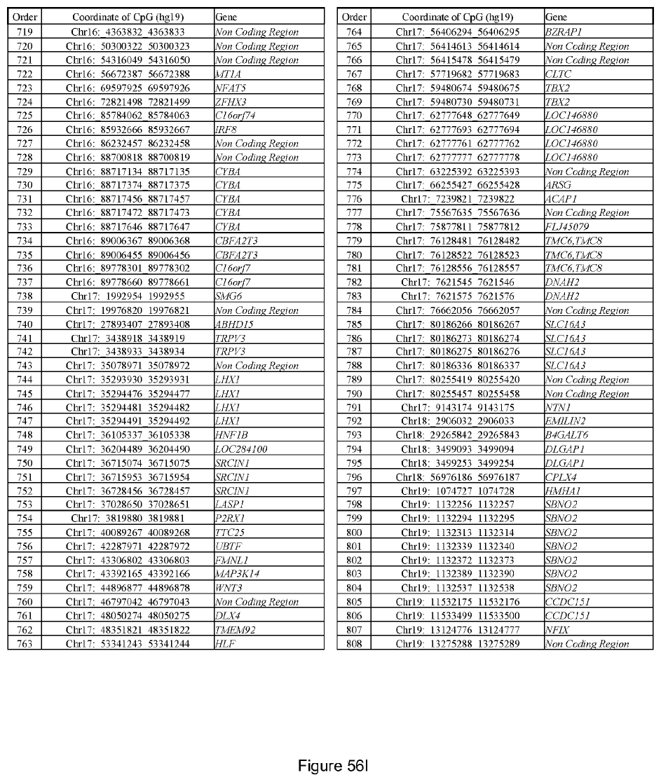

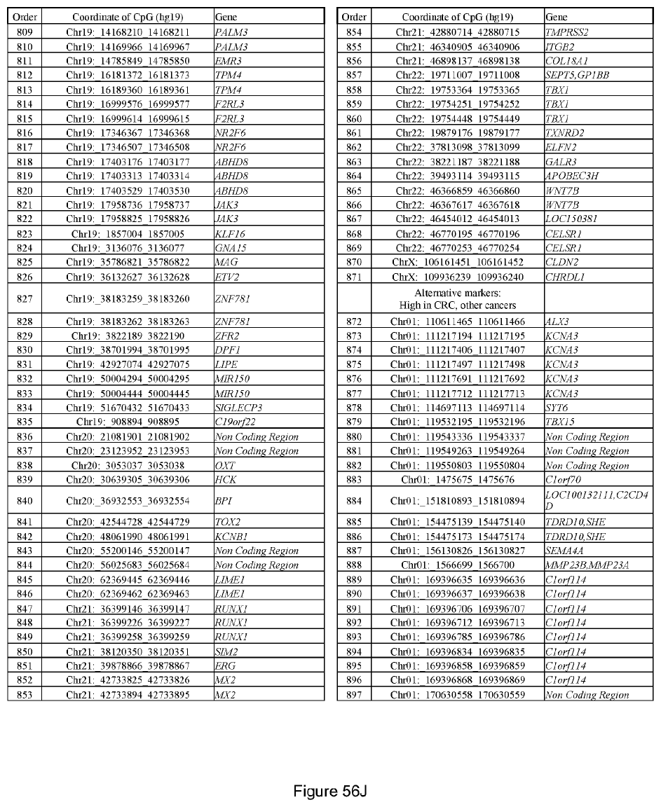

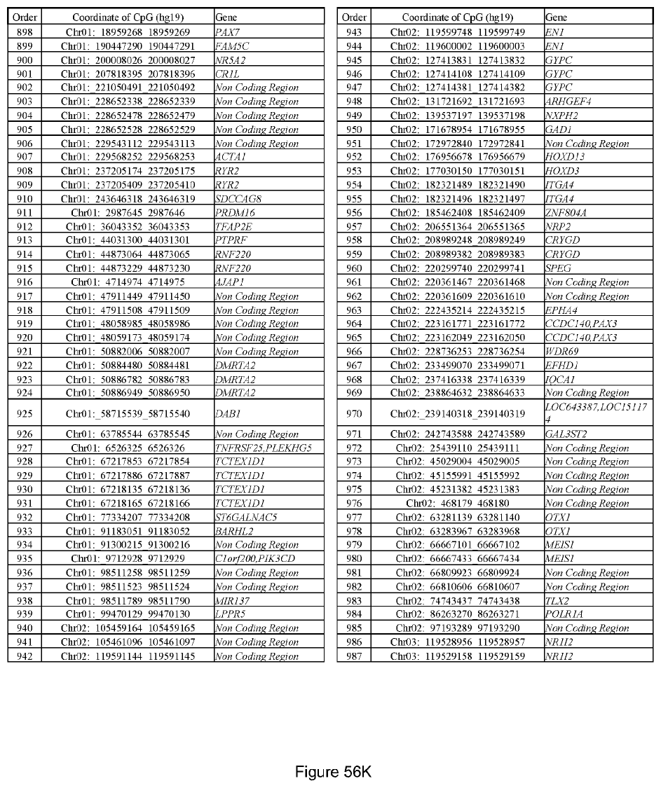

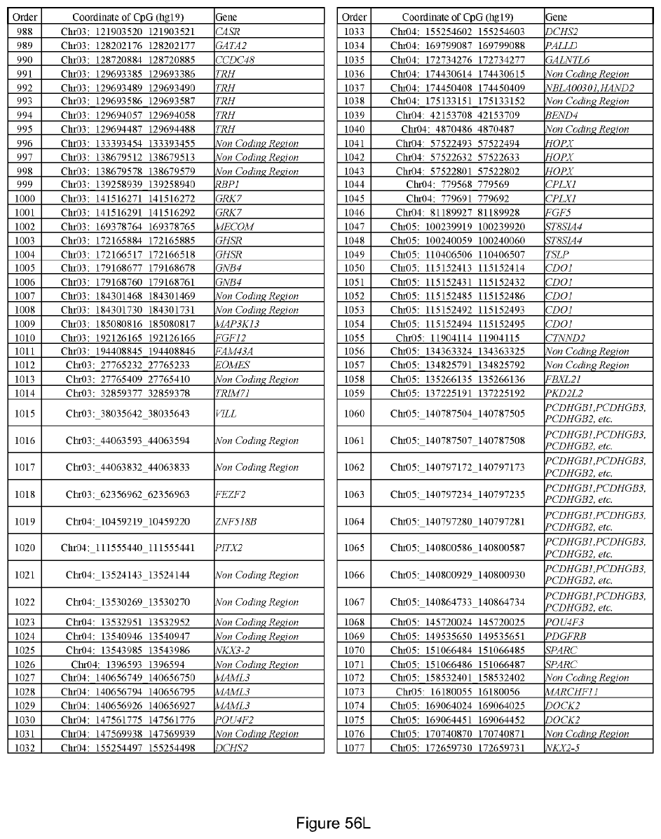

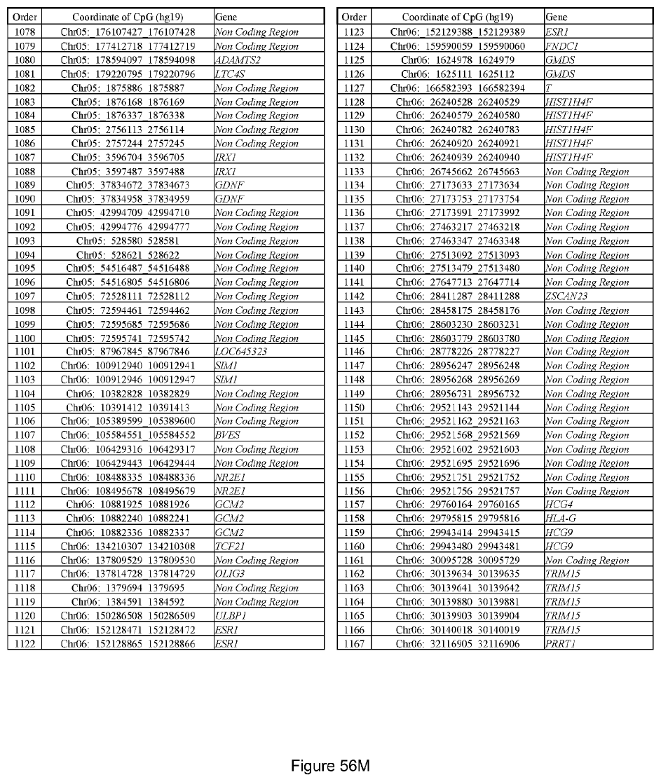

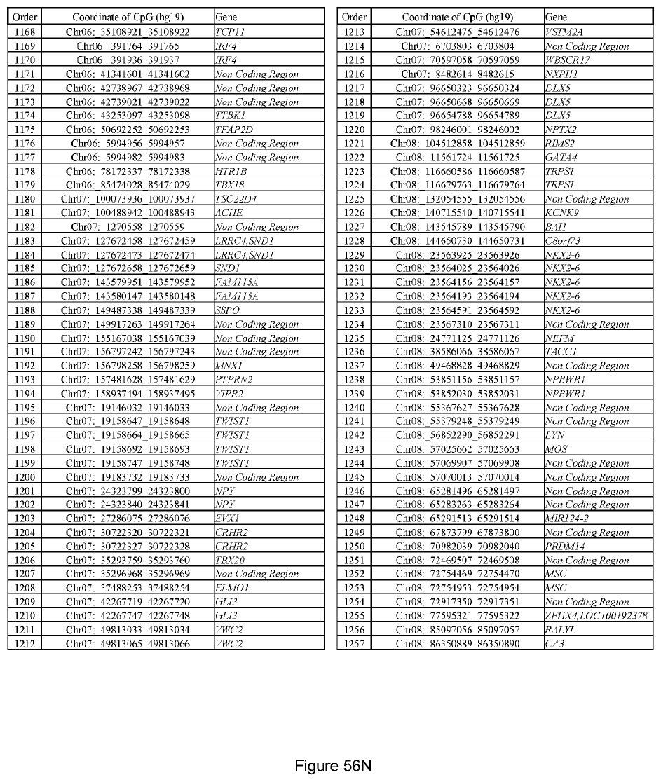

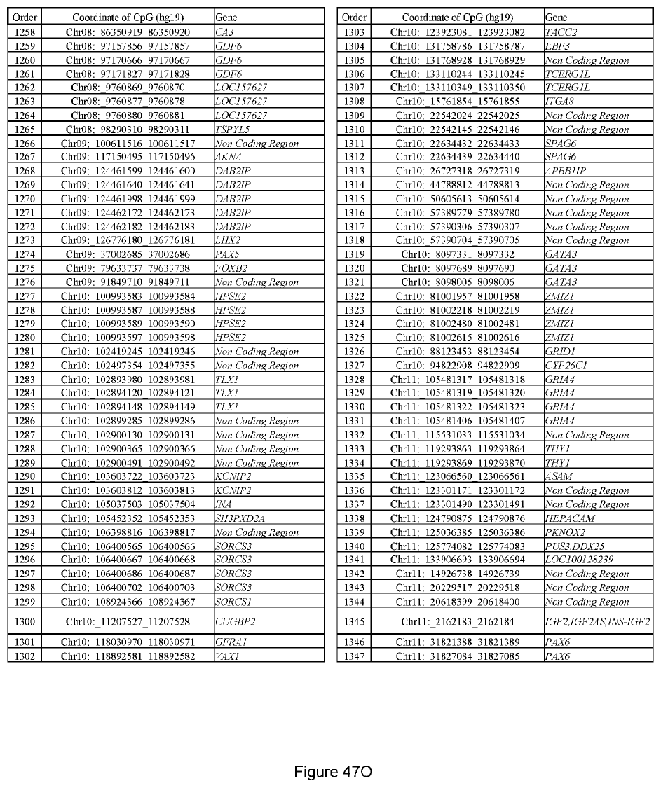

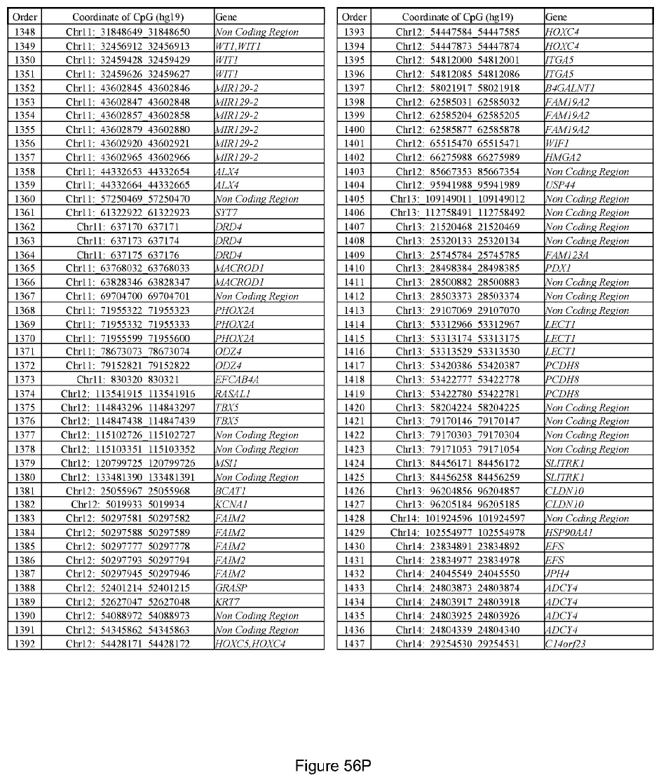

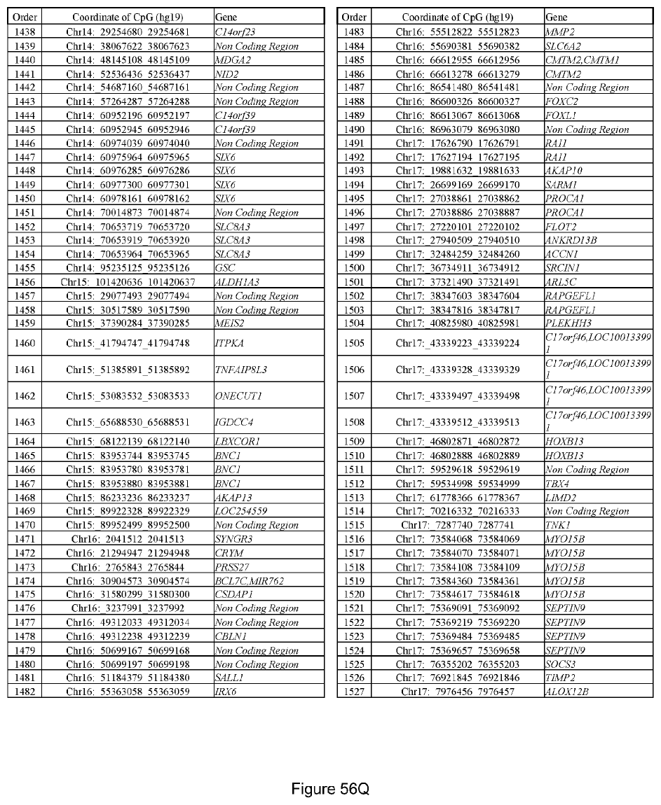

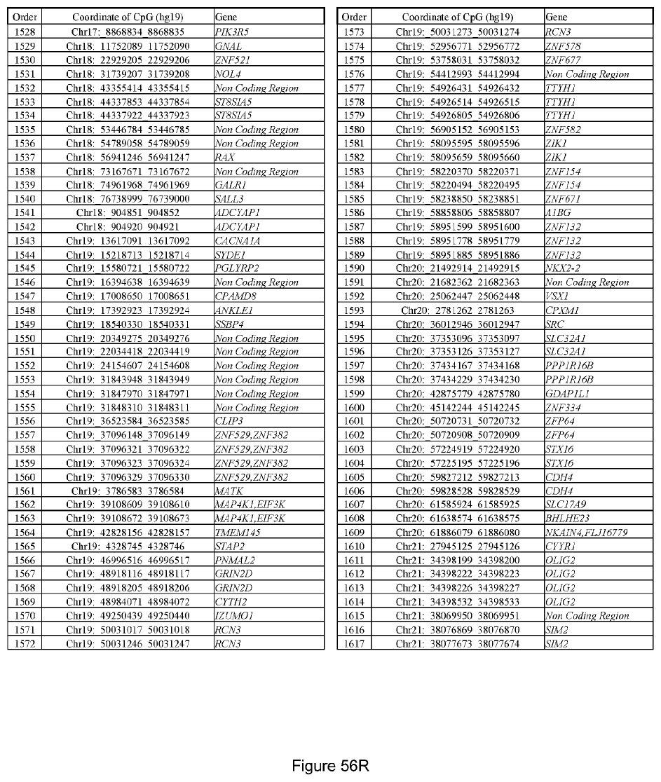

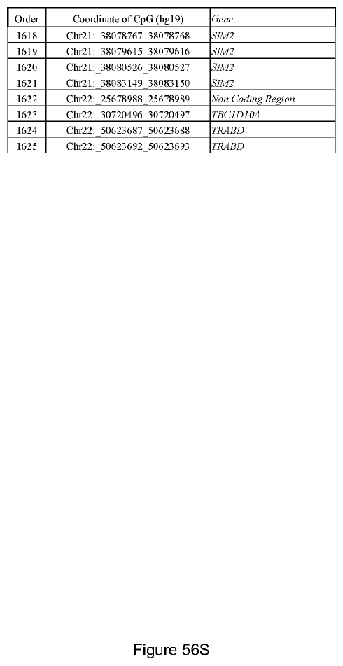

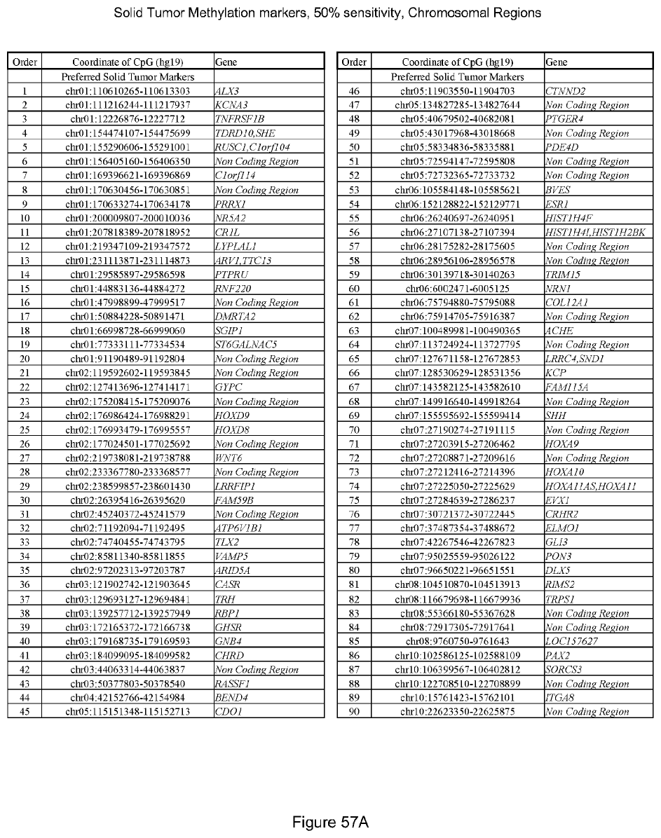

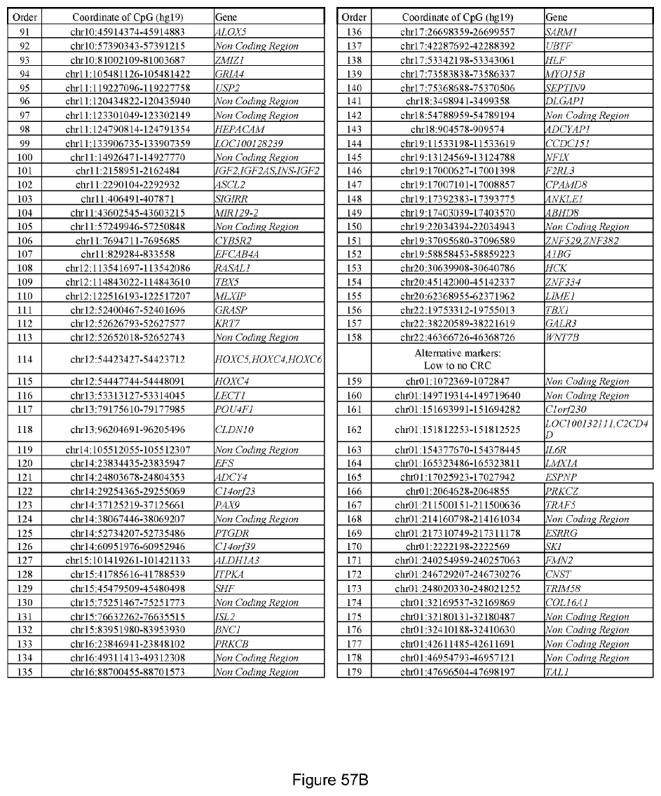

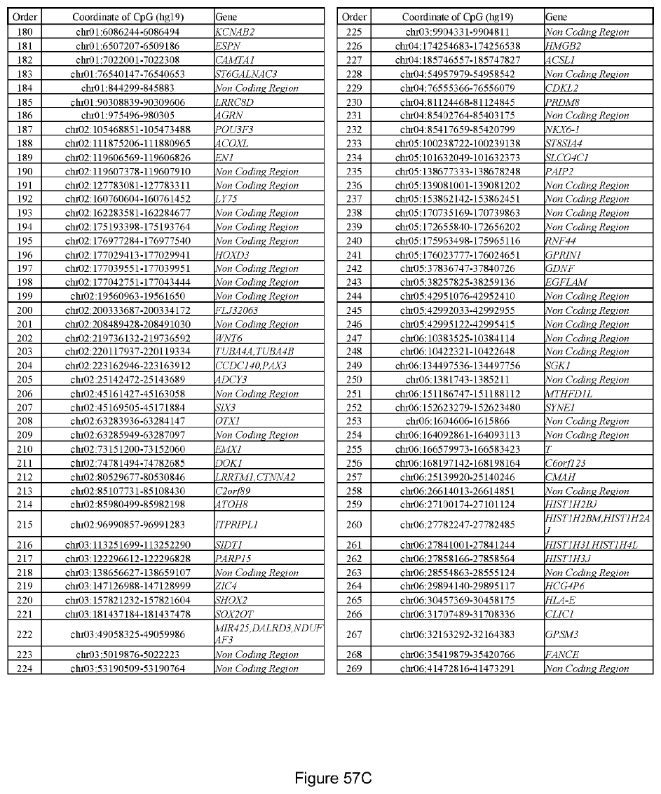

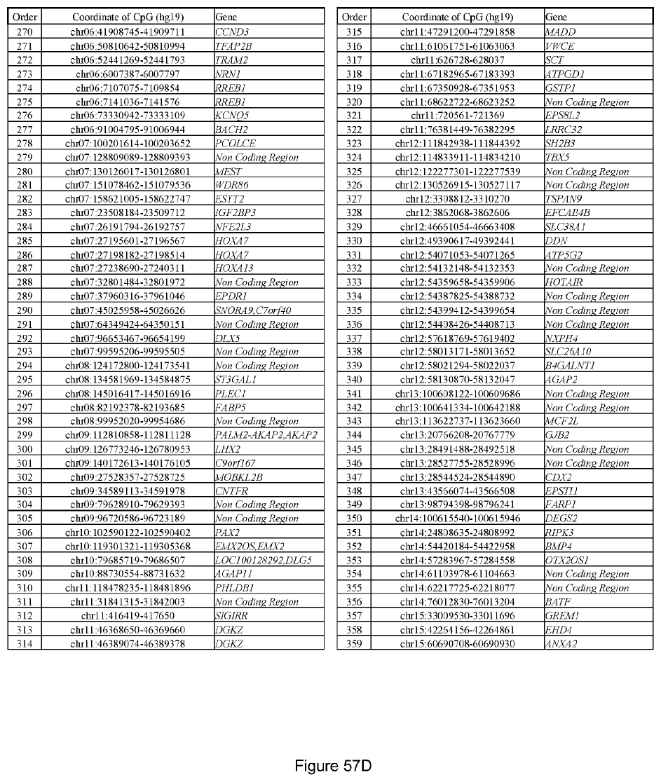

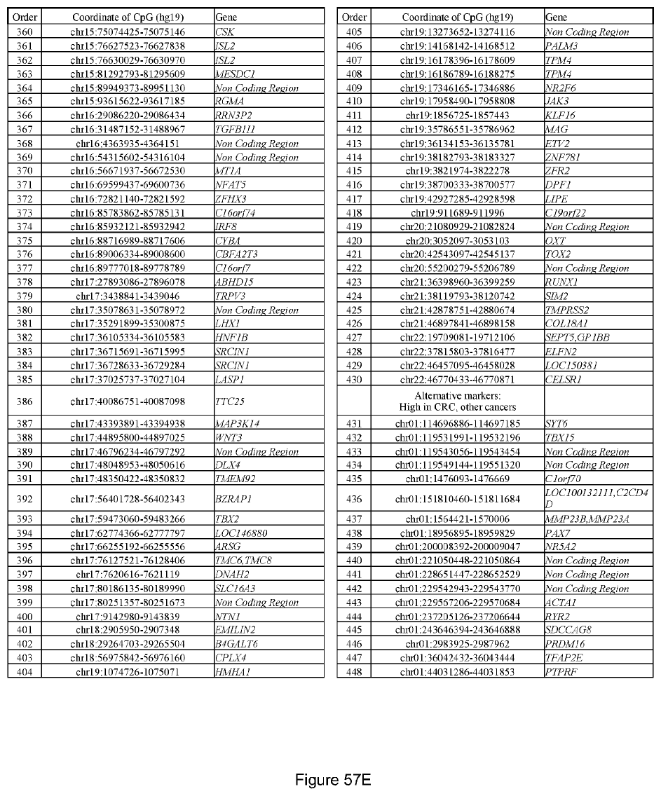

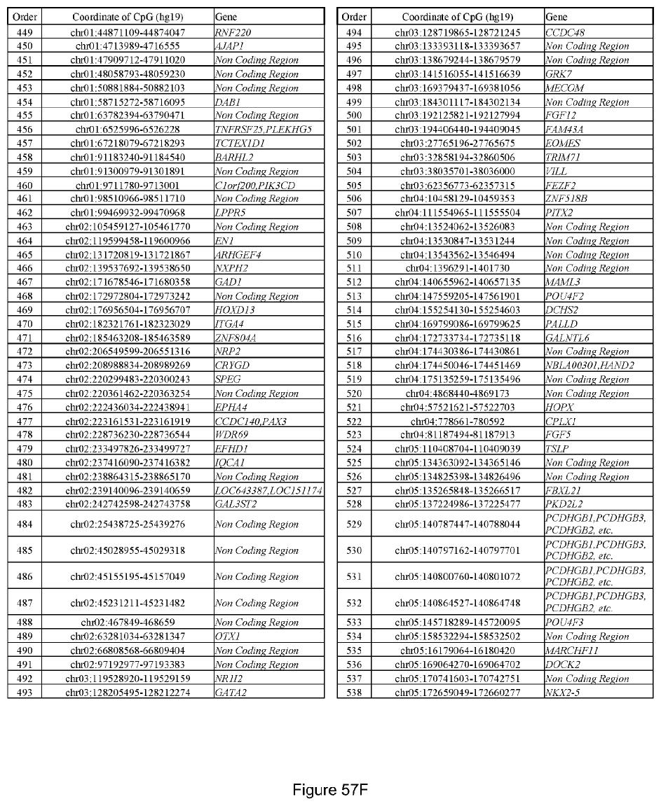

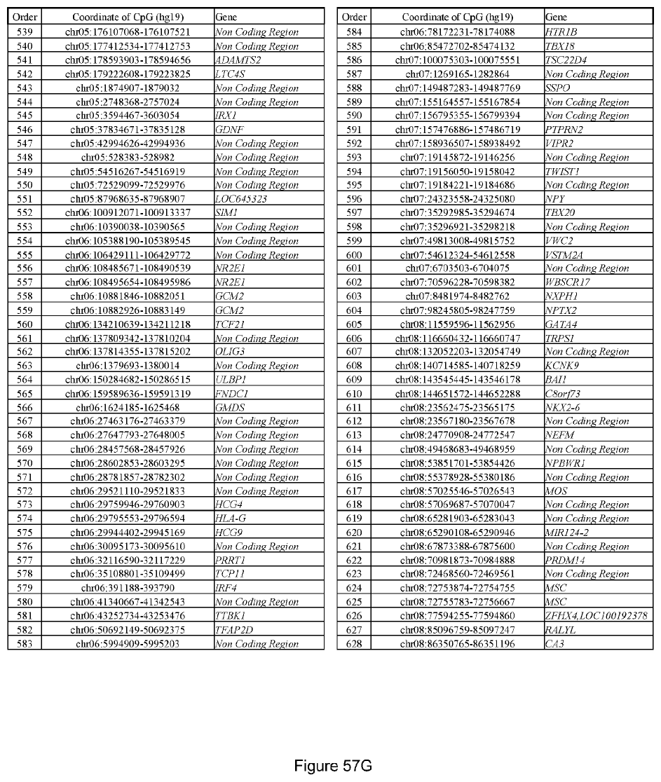

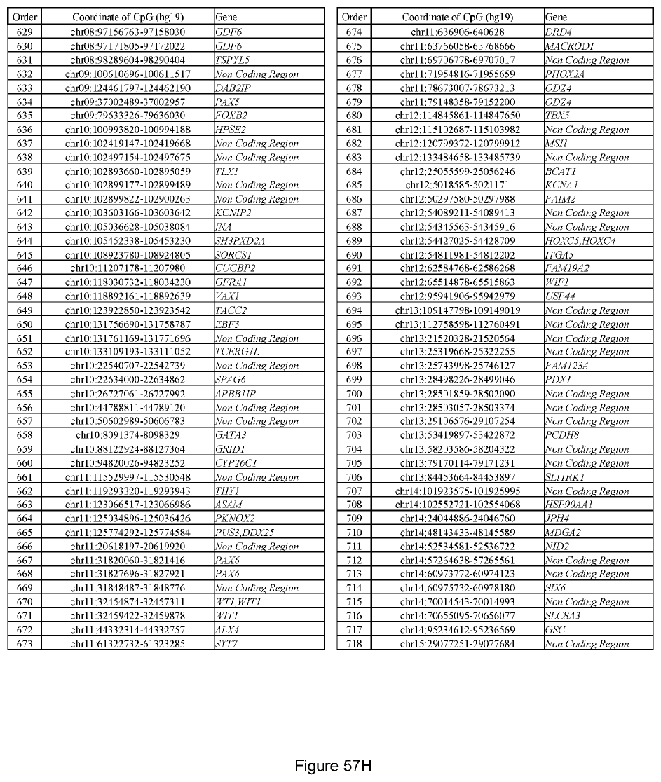

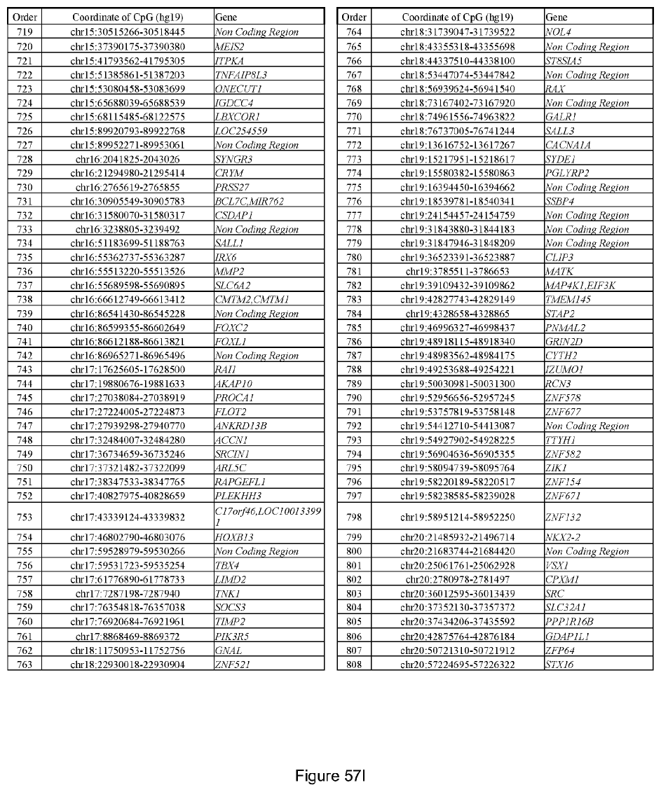

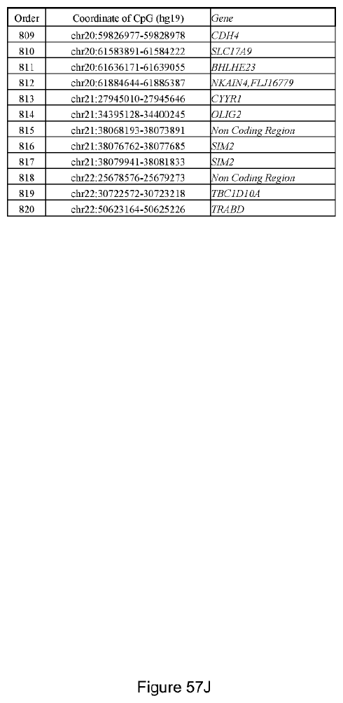

FIG. 56 provides a list of primary CpG sites that are solid tumors and tissue-specific markers, that may be used to identify the presence of solid tumors from cfDNA, DNA within exosomes, or DNA in other protected states (such as within CTCs) within the blood. FIG. 57 provides a list of chromosomal regions or sub-regions within which are primary CpG sites that are solid tumors and tissue-specific markers, that may be used to identify the presence of solid tumors from cfDNA, DNA within exosomes, or DNA in other protected states (such as within CTCs) within the blood. These lists contain preferred primary CpG sites and their flanking sites, as well as alternative markers that are low to no-CRC, and alternative markers that are high is CRC, with or without being high for other cancers as well. Primer sets for exemplary preferred and alternate methylation markers are listed in Table 46 in the experimental section.

Table 47, in the experimental section, provides simulations of the 96-marker assay, with average sensitivities of 50%, for identifying most probably group for tissue of origin, for both sexes. A set of 96 markers was assembled as above and the percentage of samples positive within each of the cancer patients in the TCGA and GEO databases was assessed. The total number of patients for each cancer analyzed are: Group 1 (colorectal, CRC-PT=395; stomach, ST-Pt=260; esophagus, ES-Pt=185); Group 2 (breast, BR-Pt=668; endometrial, END-Pt=431; ovarian, OV-Pt=79; cervical, CERV-Pt=307; uterine, UTCS-Pt=57); Group 3 (lung adenocarcinoma, LUAD=450; lung squamous cell carcinoma, LUSC=372; head & neck, HNSC-Pt=528); Group 4 (prostate, PROS-Pt=192; bladder, BLAD-Pt=412); and Group 5 (liver, LIV-Pt=377; pancreatic, PANC-Pt=184; and gall bladder, BILE-Pt=36). The columns reflect the total percent patients positive for each of the markers divided by the total number of markers used—for the first row of all cancers, that would be 96 markers. Thus, on average, of the 96 markers chosen, the number of average sensitivity scores are: Group 1 (colorectal=44, stomach=45, esophagus=40); Group 2 (breast=38, endometrial=40, ovarian=22, cervical=39, uterine=33); Group 3 (lung adenocarcinoma=31, lung squamous cell carcinoma=31, head & neck=33); Group 4 (prostate=45, bladder=36); and Group 5 (liver=38, pancreatic=27, gall bladder=47). This translates into the following number of marker equivalents with average sensitivities of 50% (=96×score/50); (colorectal=85 marker equivalents; stomach=86 marker equivalents; esophagus=78 marker equivalents); Group 2 (breast=74 marker equivalents; endometrial=76 marker equivalents; ovarian=42 marker equivalents; cervical=75 marker equivalents; uterine=64 marker equivalents); Group 3 (lung adenocarcinoma=60 marker equivalents; lung squamous cell carcinoma=59 marker equivalents; head & neck=64 marker equivalents); Group 4 (prostate=86 marker equivalents; bladder=70 marker equivalents); and Group 5 (liver=74 marker equivalents; pancreatic=51 marker equivalents; gall bladder=91 marker equivalents). Thus, cancers were well represented, ranging from 42 to 91 marker equivalents for the different cancer types, and all well above the minimum of 36 markers with average sensitivities of 50%.

The above numbers translate into the following number of marker equivalents with average sensitivities of 66% (=96×score/66); (colorectal=65 marker equivalents; stomach=65 marker equivalents; esophagus=59 marker equivalents); Group 2 (breast=56 marker equivalents; endometrial=58 marker equivalents; ovarian=32 marker equivalents; cervical=57 marker equivalents; uterine=48 marker equivalents); Group 3 (lung adenocarcinoma=45 marker equivalents; lung squamous cell carcinoma=45 marker equivalents; head & neck=48 marker equivalents); Group 4 (prostate=65 marker equivalents; bladder=53 marker equivalents); and Group 5 (liver=56 marker equivalents; pancreatic=39 marker equivalents; gall bladder=69 marker equivalents). Thus, cancers were well represented, ranging from 32 to 69 marker equivalents for the different cancer types, and with the exception of ovarian cancer at 32, the other cancer types are above the minimum of 36 markers with average sensitivities of 66%.

The aforementioned markers were then re-ordered for each of the above cancer types such that the most prevalent markers were listed first. For example, with CRC, of the 96 markers, 54 markers gave scores above 55 (i.e. were positive in greater than 55% of the 395 patients) and 9 gave scores of between 25 and 54 (i.e. were positive for from 25% to 54% of the 395 patients). Half of the higher, and a third of the lower set, for a total of 30 markers were distributed into two marker test sets, designated “CRC1” and “CRC2” (Table 47, rows 2 & 3). These marker sets would reflect an ideal result if half the markers with the potential to be positive are detected in the assay. This does not account for the chances that earlier stage tumors would have a lower number of marker molecules in the plasma, and thus consequently the actual number of markers positive would be less than the ideal result in this simulation. The percent of patients positive for each of the cancers were recorded and then divided by the total number of markers used for that cancer type. As anticipated, when selecting markers for a given tumor type, those markers should give a higher score than the average, i.e. 66 for CRC in each of the two sets of selected 30 markers, compared with a score of 44 for the unselected 96 markers. These markers form a diagonal across Table 47 and are highlighted in bold and light grey background.

For each column, marker sets that are in the same range or higher than the number of positive markers for that cancer type are also shown with a light grey background. For example, a patient with colorectal, stomach, or esophageal cancer will be scored as potentially positive with stomach cancer. This makes sense as the markers for these three cancers ovelap (i.e., they all bin to Group 1). They could be distinguished in step 2 of the assay on the group 1 markers, where these markers are more cancer types specific and tease out the most probable cancer type. Evaluation of the ST-Pt column shows that simulations for one of the two LUAD, BLAD, and both PANC also gave scores that might be interpreted as stomach cancer. Thus, the first step is not always able to pinpoint what Groups should be tested in the second step of the assay. However, most of the ambiguity is within group members (i.e. Group 2), and this makes sense, since the markers were chosen to maximize the ability to chose which groups to test in the second step.

Tables 48 and 49 (see prophetic experimental section) takes the aforementioned results in the simulations in Table 47 and multiplies them by the percent incidence of the given cancer type for that gender (see tables 37 and 38 respectively), and the result is adjusted to the same order of magnitude (multiple by 10). The concept is for the physician to take into account that a lower score for a high incidence cancer (such as CRC) may be a more common tissue of origin for a higher score for a low incidence cancer (such as lung squamous cell carcinoma). Tables 48 and 49 show the level of ambiguity in identifying tissue of origin is higher among female patients than among male patients, as indicated by the number of cells highlighted in grey that are not on the diagonal. In all cases, the physician will need to incorporate all data, such as smoking history, and not just molecular data to determine the most likely tissue of origin before sending the patient to confirmatory imaging.

Tables 50, 51, and 52 (see prophetic experimental section) takes the aforementioned results in the simulations in Tables 47, 48, and 49 and determines the percent deviation from the neutral result by taking the percentage of (=score specific cancer type simulation/score all cancer for that type−1). Thus, the first row of each of these tables should be 0%. Again, those percentages that are higher than, or in the same range as, the percentages across the diagonal are highlighted in light gray. While this set of marker selection may be less than ideal for distinguishing esophageal or gall bladder cancers as the tissue of origin, they are nevertheless quite informative for guiding the physician to which groups of the Step 2 assays should be tested. This simple scoring may be augmented by using AI approaches based on a database of results with clinical samples using the aforementioned 96-marker set.

For the second step of the assay, one, two, or more of the following groups will be tested, each group with a set of 64 markers that on average comprise of at least 36 markers with 50% sensitivity that covers each of the aforementioned 16 types of solid tumors in the following groups: Group 1 (colorectal, stomach, and esophagus); Group 2 (breast, endometrial, ovarian, cervical, and uterine); Group 3 (lung and head & neck); Group 4 (prostate and bladder); and Group 5 (liver, pancreatic, or gall bladder). These Group-specific and cancer type-specific markers include, but are not limited to, cancer-specific microRNA markers, cancer-specific ncRNA and lncRNA markers, cancer-specific exon transcripts, tumor-associated antigens, cancer protein markers, protein markers that can be secreted by solid tumors into the blood, common mutations, primary CpG sites that are solid tumor and tissue specific markers, chromosomal regions or sub-regions within which are primary CpG sites that are solid tumor and tissue specific markers, and primary and flanking CpG sites that are solid tumor and tissue specific markers. Methods for detecting said markers have been discussed supra, and listing of these markers are described for each of the groups below as well as in the corresponding figures.

Group 1 (colorectal, stomach, and esophagus): Blood-based, colorectal, stomach, and esophageal cancer-specific microRNA markers that may be used to distinguish group 1 from other groups include, but are not limited to: (mir ID, Gene ID): hsa-mir-624, MIR624. This miRNA was identified through analysis of TCGA microRNA datasets, and may be present in exosomes, tumor-associated vesicles, Argonaute complexes, or other protected states in the blood.

Blood-based, colorectal, stomach, and esophageal cancer-specific ncRNA and lncRNA markers that may be used to distinguish group 1 from other groups include, but are not limited to: [Gene ID, Coordinate (GRCh38)], ENSEMBL ID: LINC01558, chr6:167784537-167796859, ENSG00000146521.8. This ncRNA was identified through comparative analysis of various publicly available Affymetrix Exon ST CEL data, which were aligned to GENCODE annotations to generate ncRNA and lncRNA transcriptome datasets. Such lncRNA and ncRNA may be enriched in exosomes or other protected states in the blood.

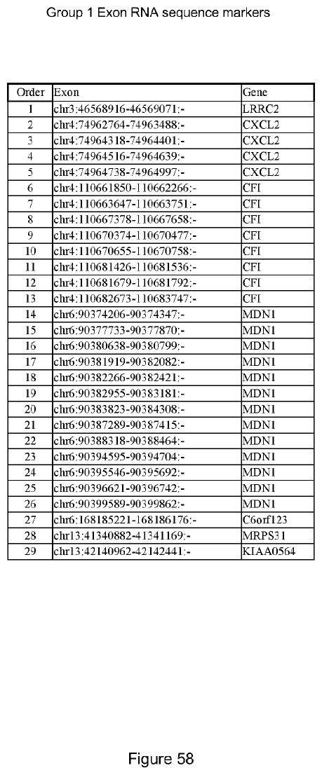

In addition, FIG. 58 provides a list of blood-based colorectal, stomach, and esophageal cancer-specific exon transcripts that may be enriched in exosomes, tumor-associated vesicles, or other protected states in the blood.

Colorectal, stomach, and esophageal cancer protein encoding markers that may be used to distinguish group 1 from other groups include, but are not limited to: (Gene Symbol, Chromosome Band, Gene Title, UniProt ID): SELE, 1q22-q25, selectin E, P16581; OTUD4, 4q31.21, OTU domain containing 4, Q01804; BPI, 20q11.23, bactericidal/permeability-increasing protein, P17213; ASB4, 7q21-q22, ankyrin repeat and SOCS box containing 4, Q9Y574; C6orf123, 6q27, chromosome 6 open reading frame 123, Q9Y6Z2; KPNA3, 13q14.3, karyopherin alpha 3 (importin alpha 4), O00505; NUP98, 11p15, nucleoporin 98 kDa, P52948, identified through mRNA sequences, protein expression levels, protein product concentrations, cytokines, or autoantibody to the protein product arising from colorectal, stomach, and esophageal cancers, which may be identified in the blood, either within exosomes, other protected states, tumor-associated vesicles, or free within the plasma.

Protein markers that can be secreted by colorectal, stomach, and esophageal cancer into the blood, and may be used to distinguish group 1 from other groups include, but are not limited to: (Protein name, UniProt ID); Bactericidal permeability-increasing protein (BPI) (CAP 57), P1721. A comparative analysis was performed across various TCGA datasets (tumors, normals), followed by an additional bioinformatics filter (Meinken et al., “Computational Prediction of Protein Subcellular Locations in Eukaryotes: an Experience Report,” Computational Molecular Biology 2(1):1-7 (2012), which is hereby incorporated by reference in its entirety), which predicts the likelihood that the translated protein is secreted by the cells.

The distribution of mutations in colorectal, stomach, and esophageal cancer are available in the public COSMIC database, with the most common being: APC (APC regulator of WNT signaling pathway), ATM (ATM serine/threonine kinase), CSMD1 (CUB and Sushi multiple domains 1), DNAH11 (dynein axonemal heavy chain 11), DST (dystonin), EP400 (E1A binding protein p400), FAT3 (FAT atypical cadherin 3), FAT4 (FAT atypical cadherin 4), FLG (filaggrin), GLI3 (GLI family zinc finger 3), KRAS (Ki-ras2 Kirsten rat sarcoma viral oncogene homolog), LRP1B (LDL receptor related protein 1B), MUC16 (mucin 16, cell surface associated), OBSCN (obscurin, cytoskeletal calmodulin and titin-interacting RhoGEF), PCLO (piccolo presynaptic cytomatrix protein), PIK3CA (phosphatidylinositol-4,5-bisphosphate 3-kinase catalytic subunit alpha), RYR2 (ryanodine receptor 2), SYNE1 (spectrin repeat containing nuclear envelope protein 1), TP53 (tumor protein p53), TTN (titin), and UNC13C (unc-13 homolog C).

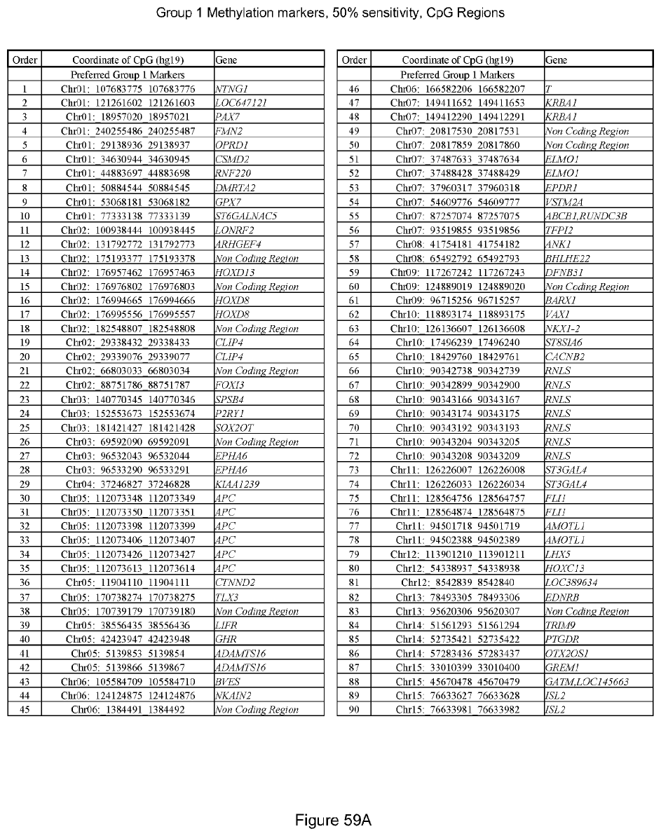

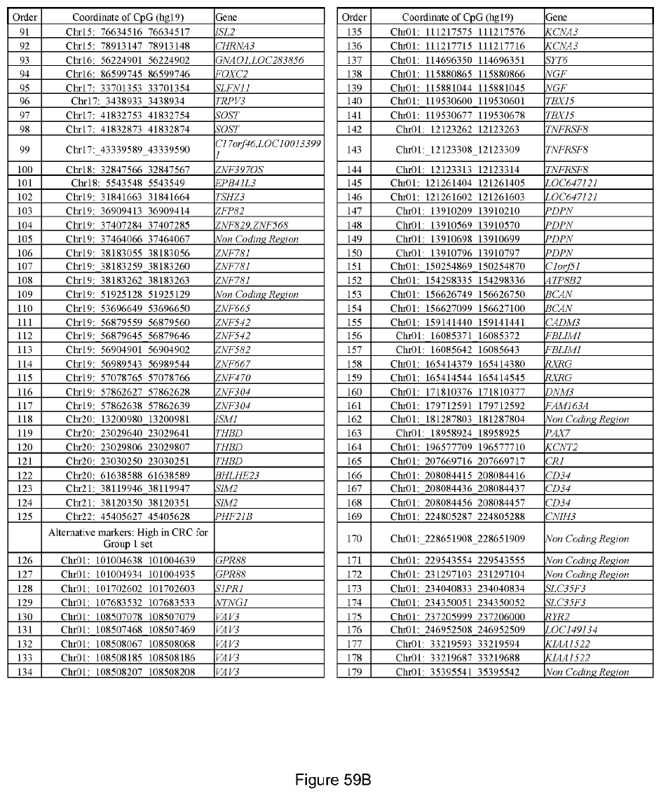

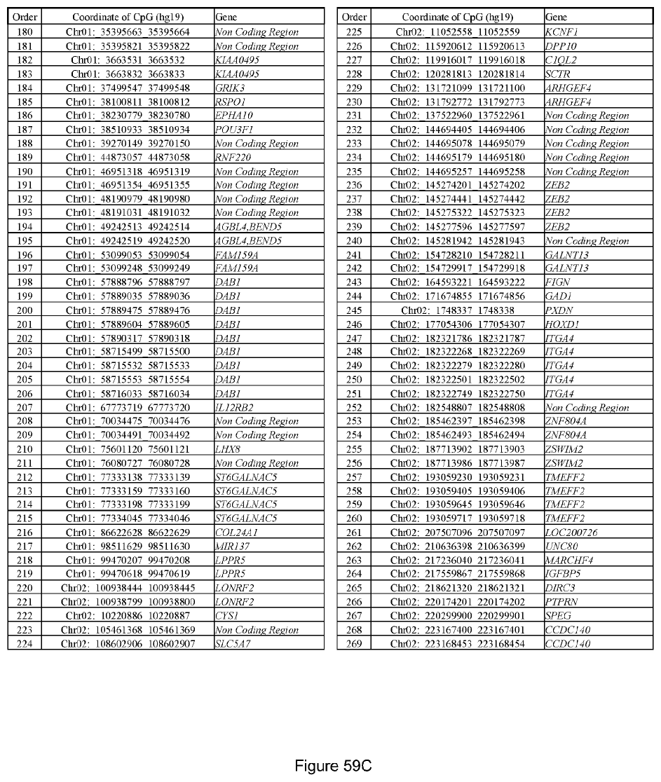

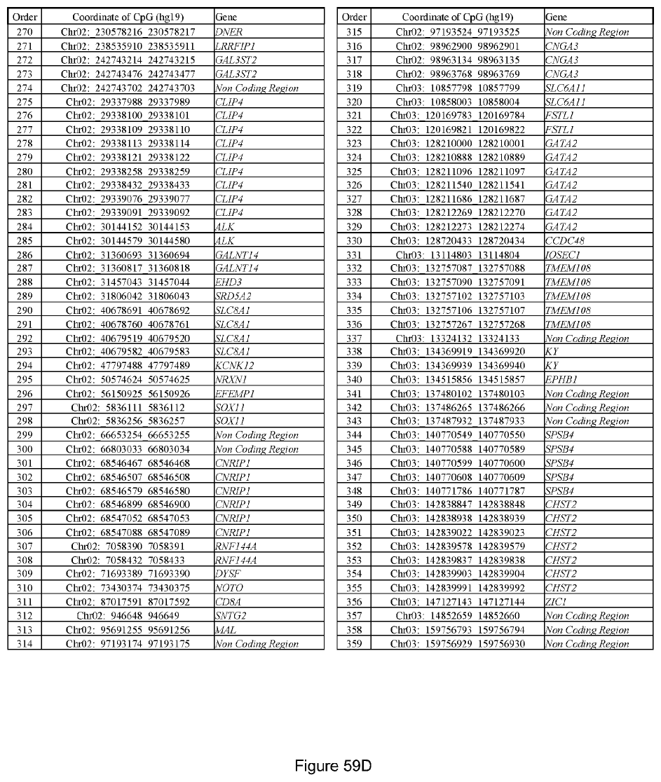

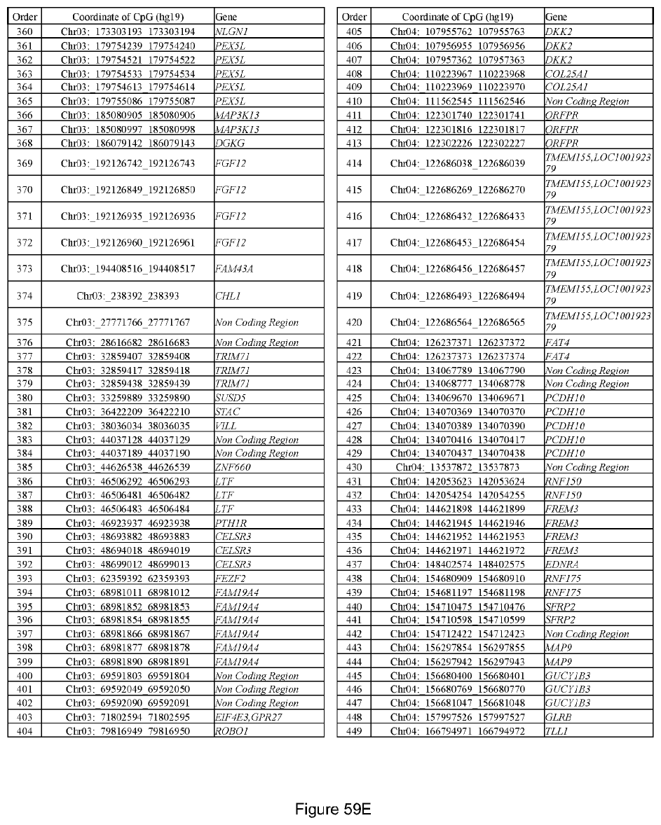

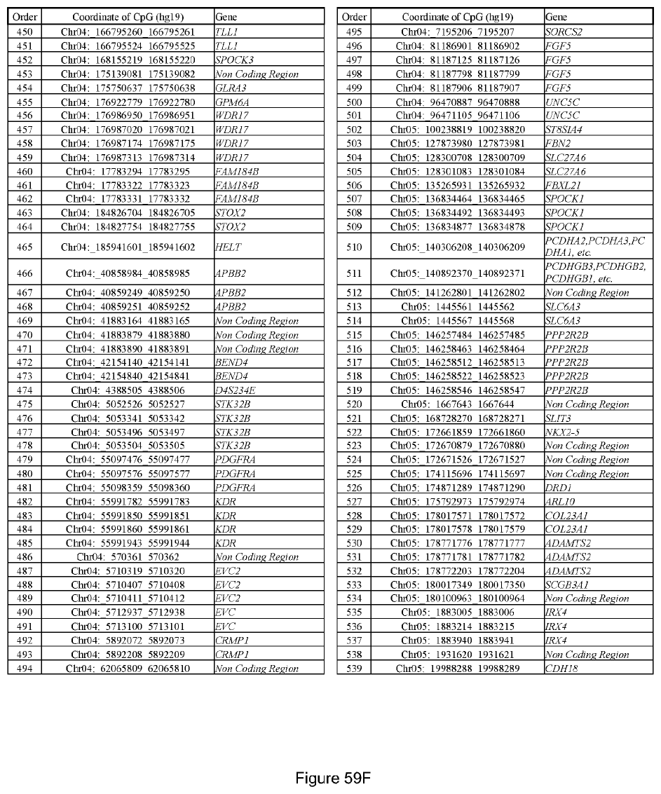

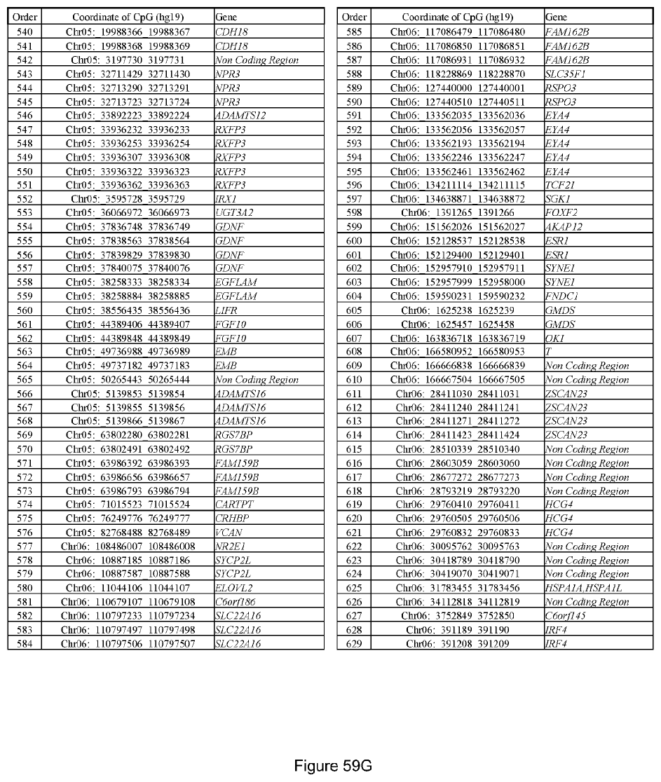

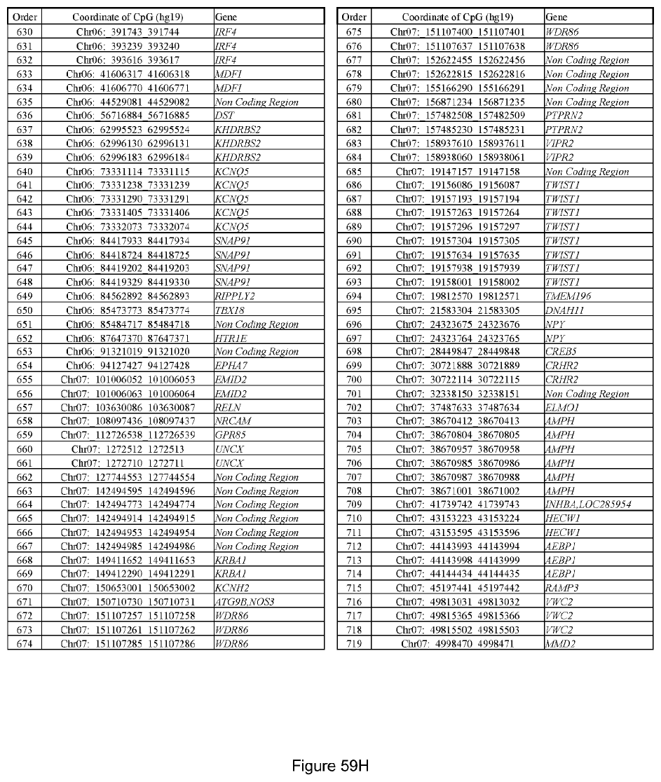

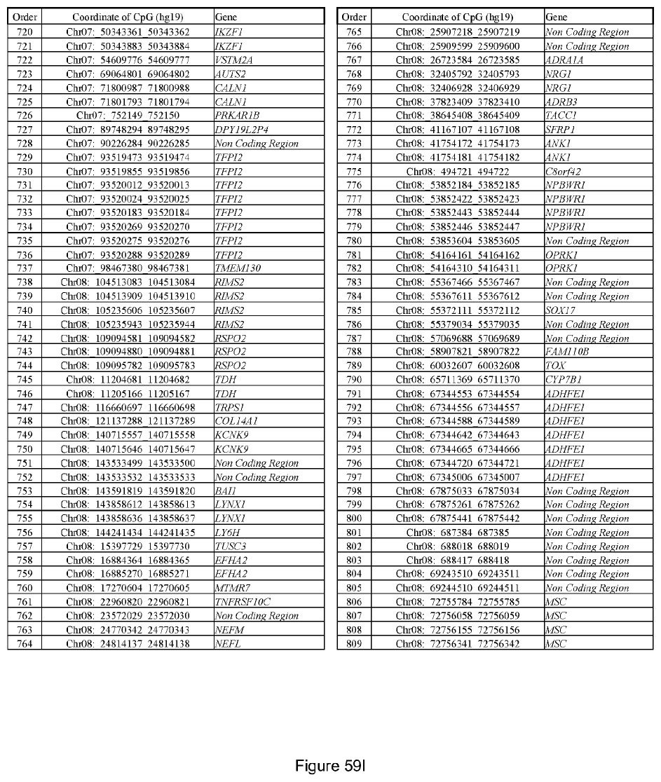

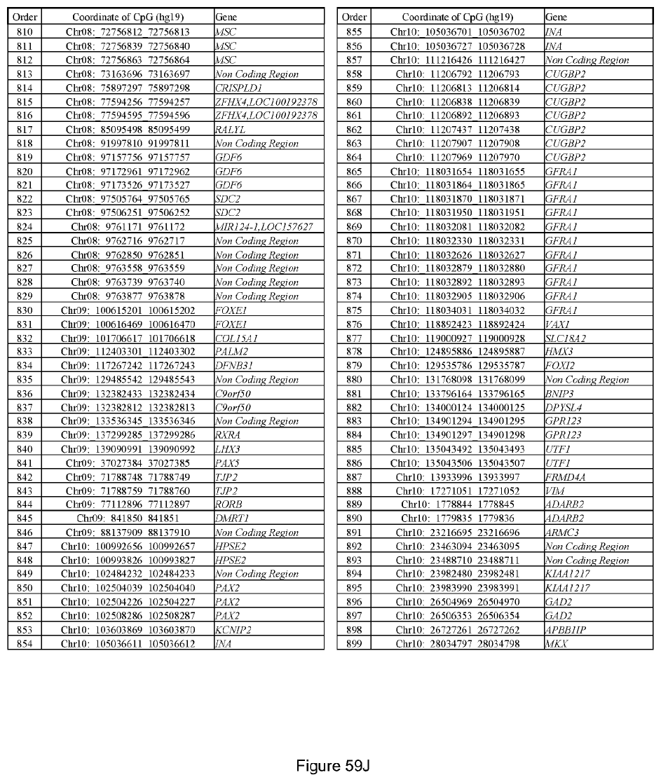

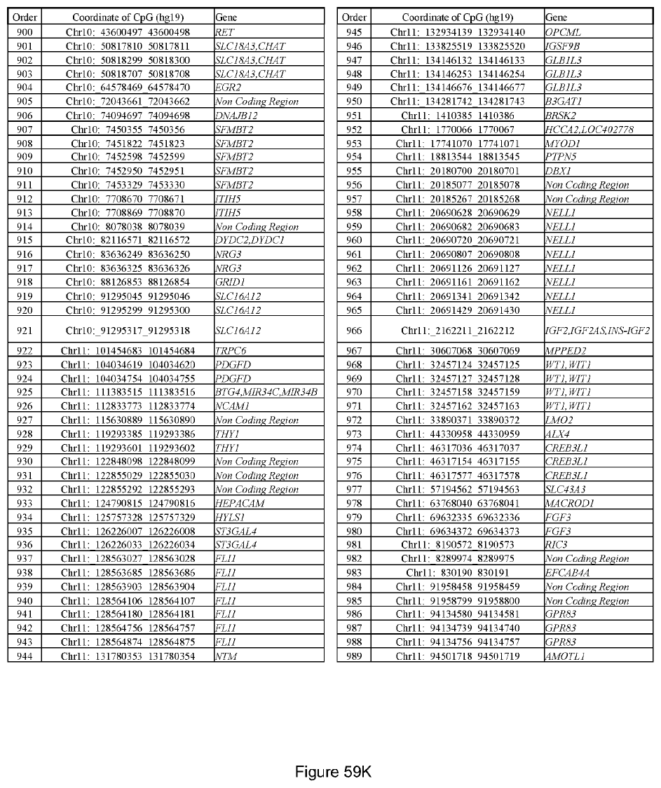

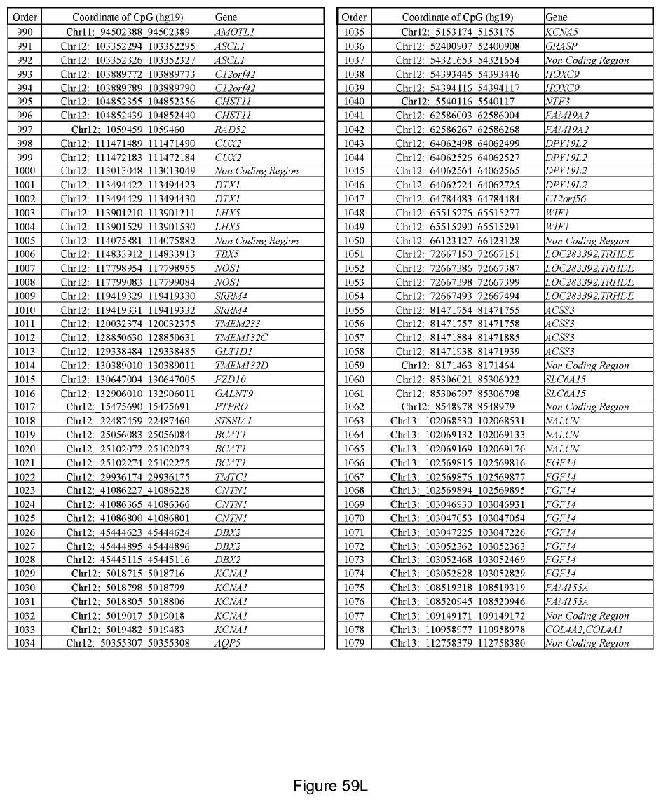

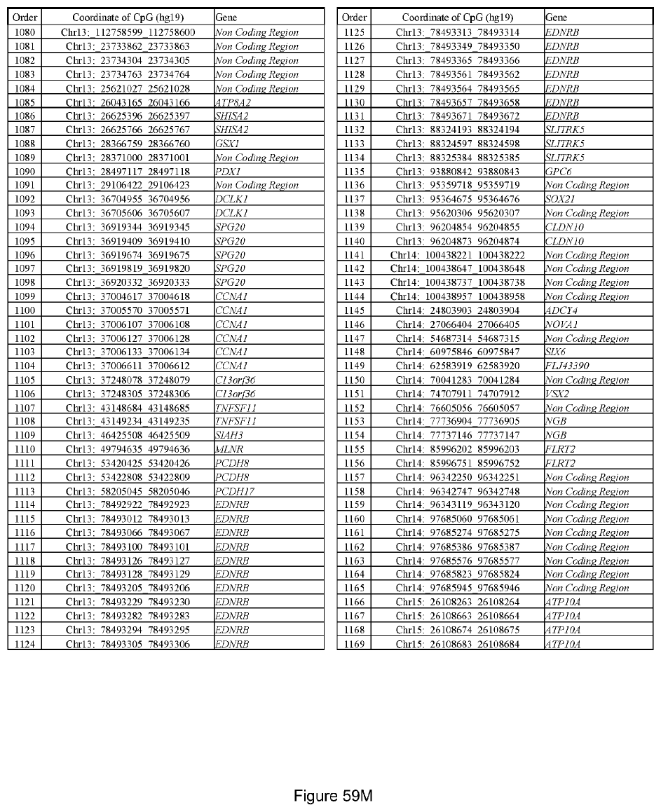

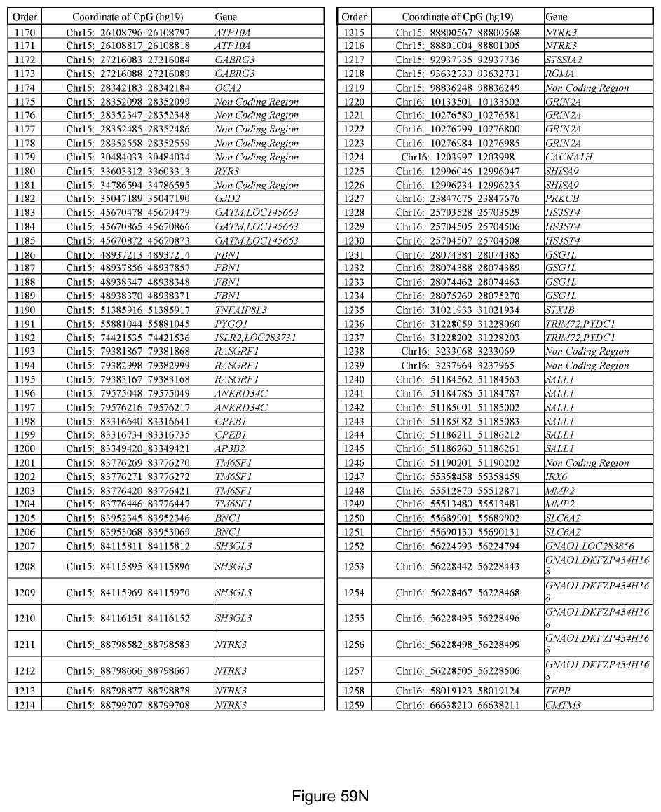

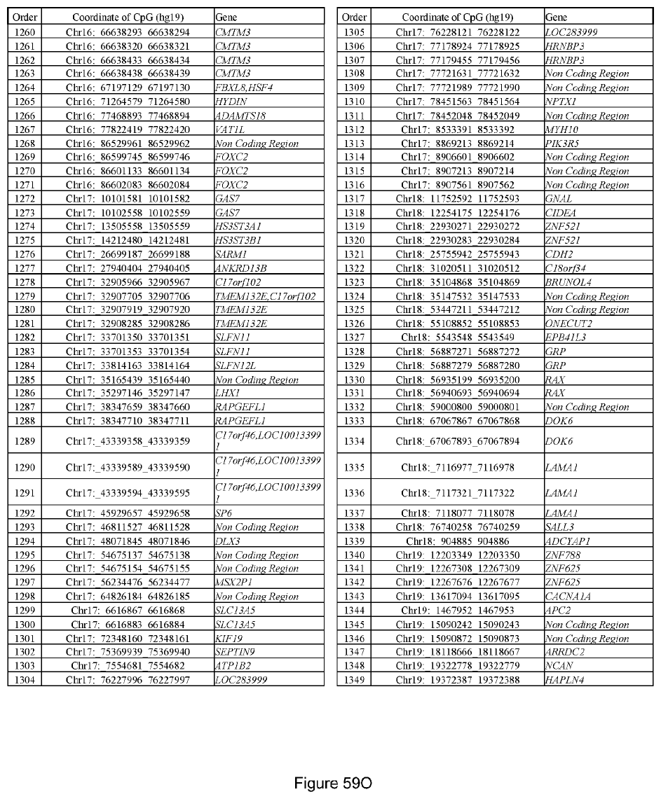

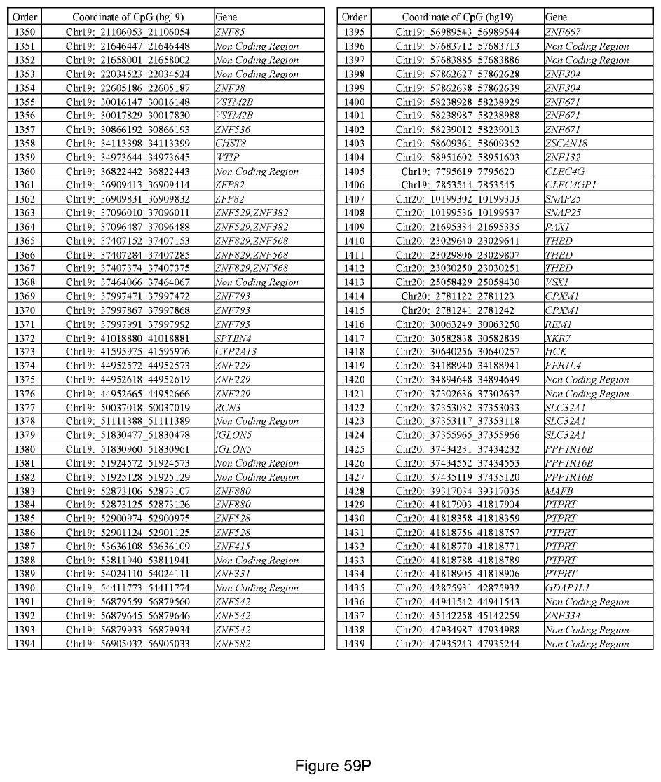

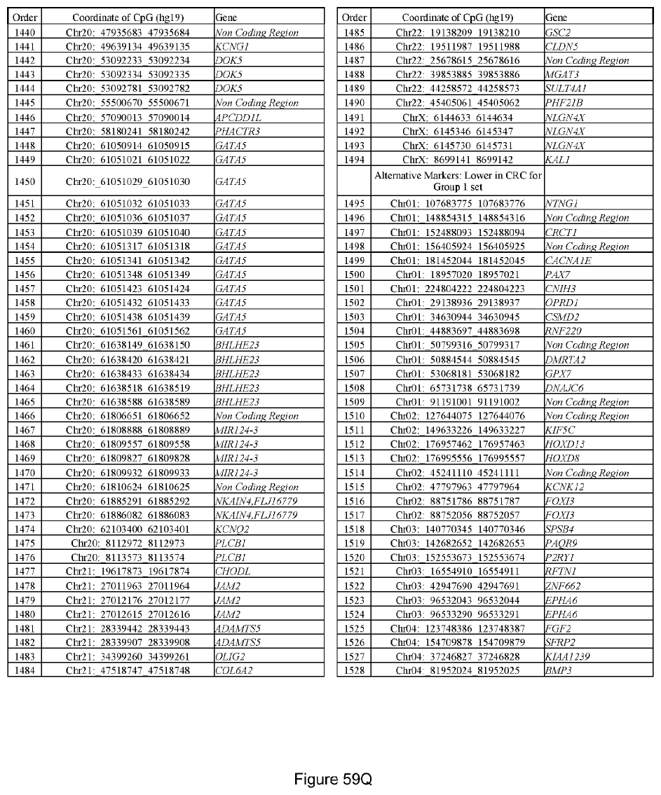

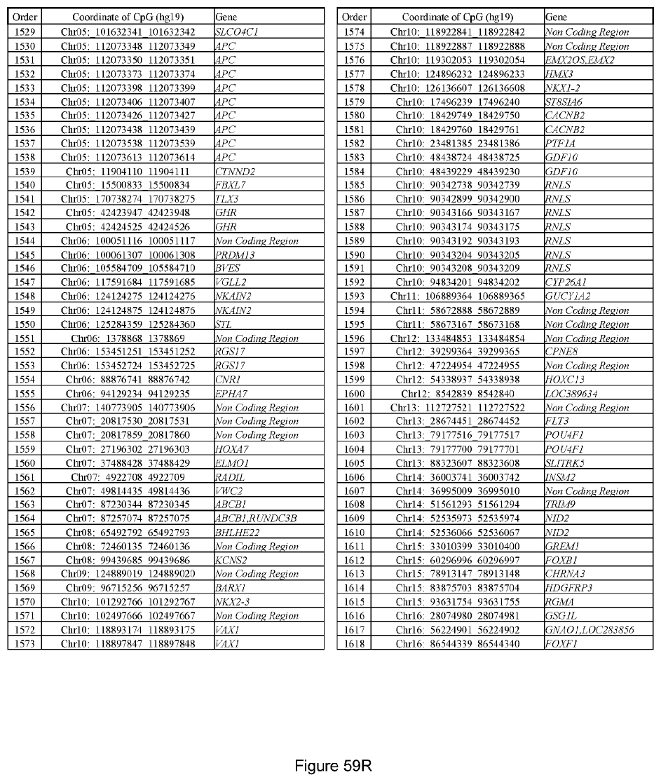

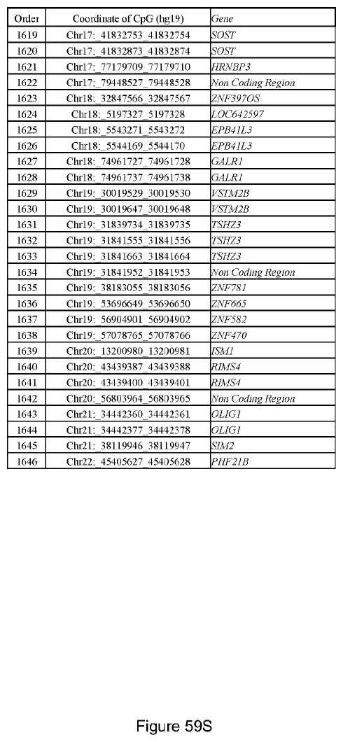

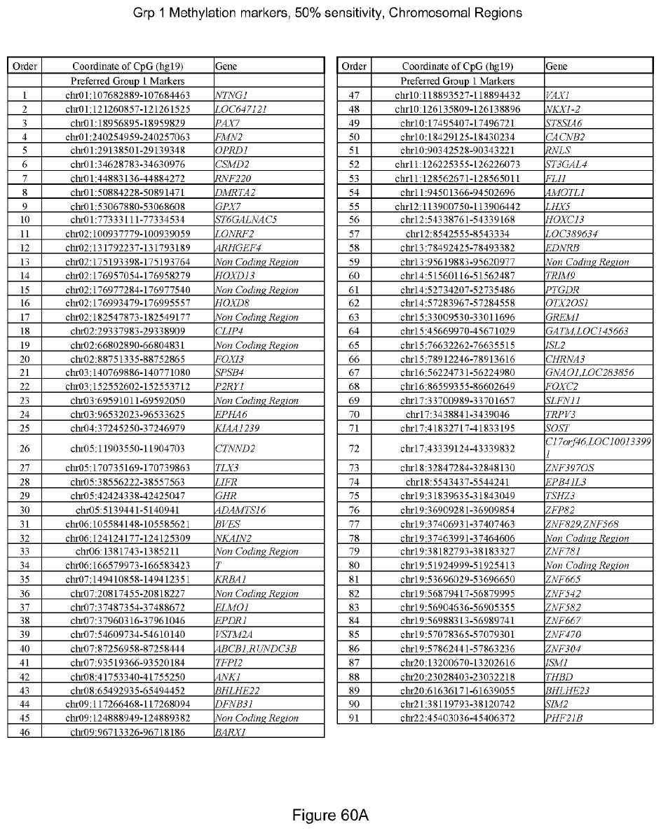

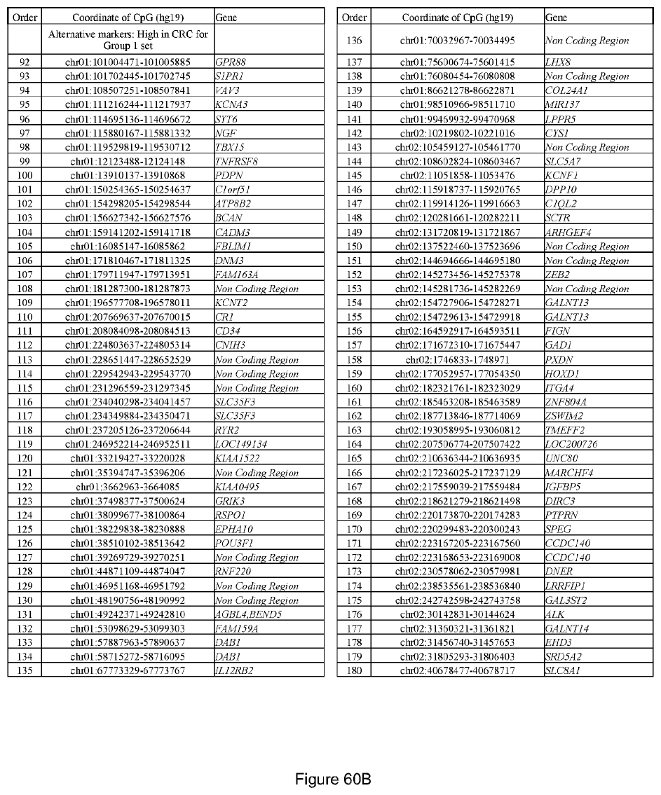

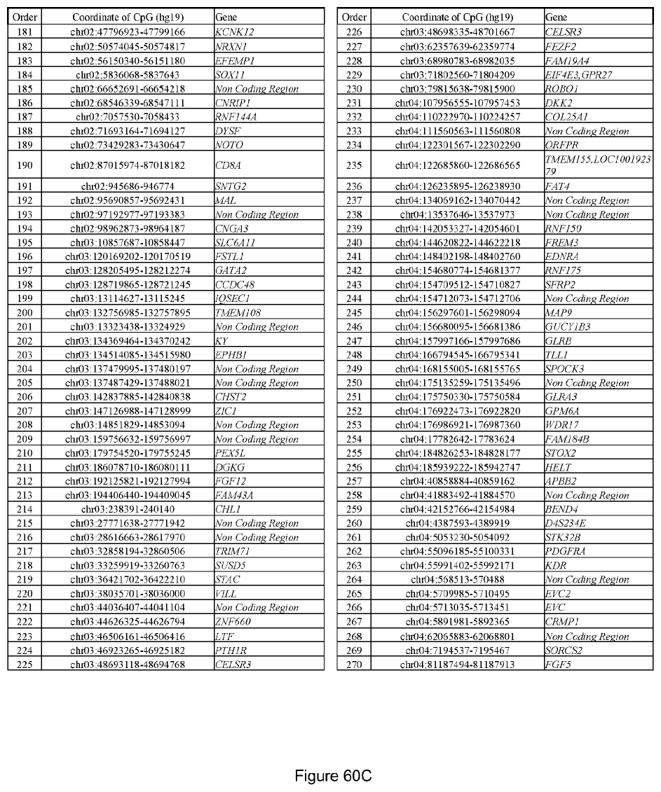

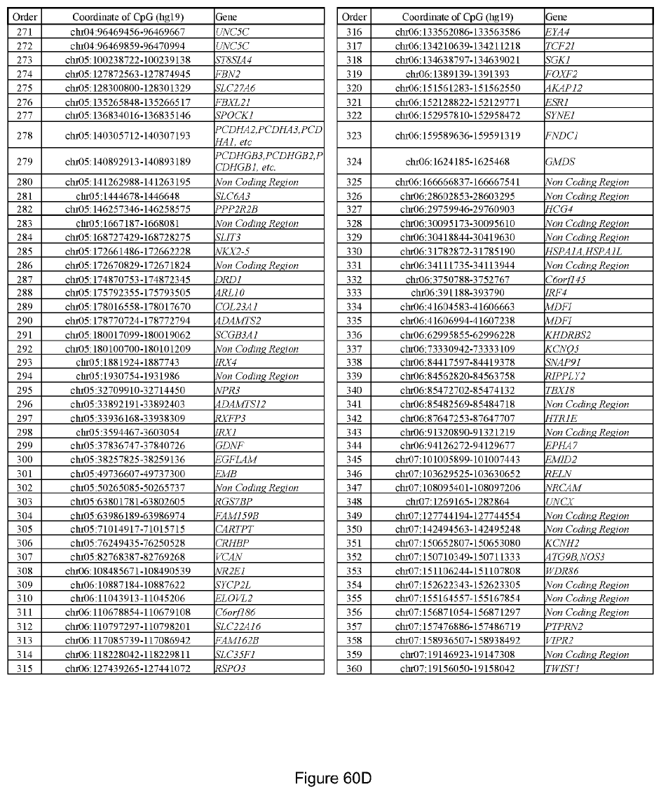

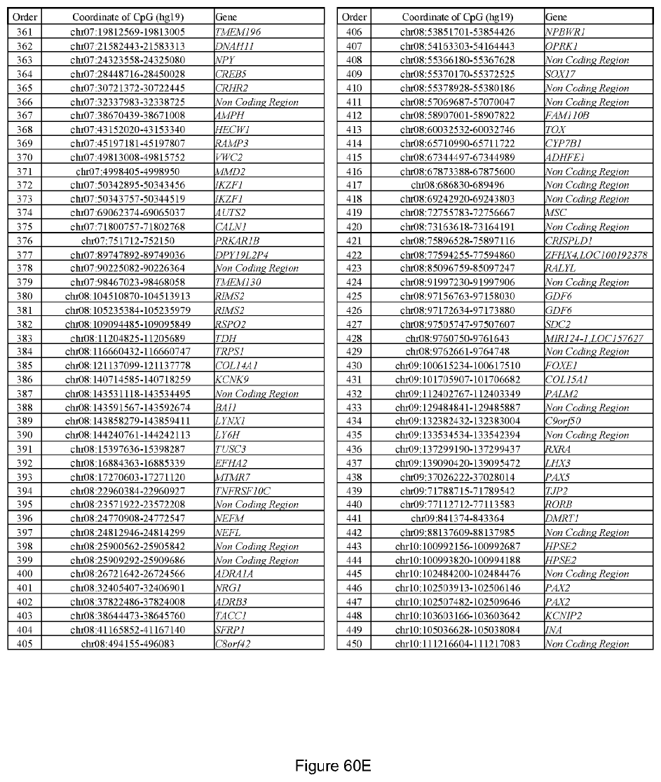

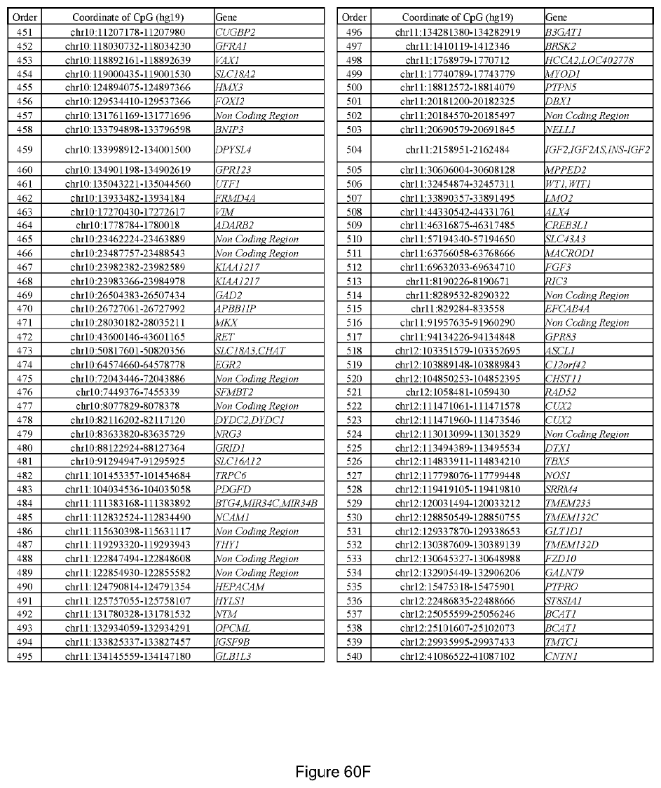

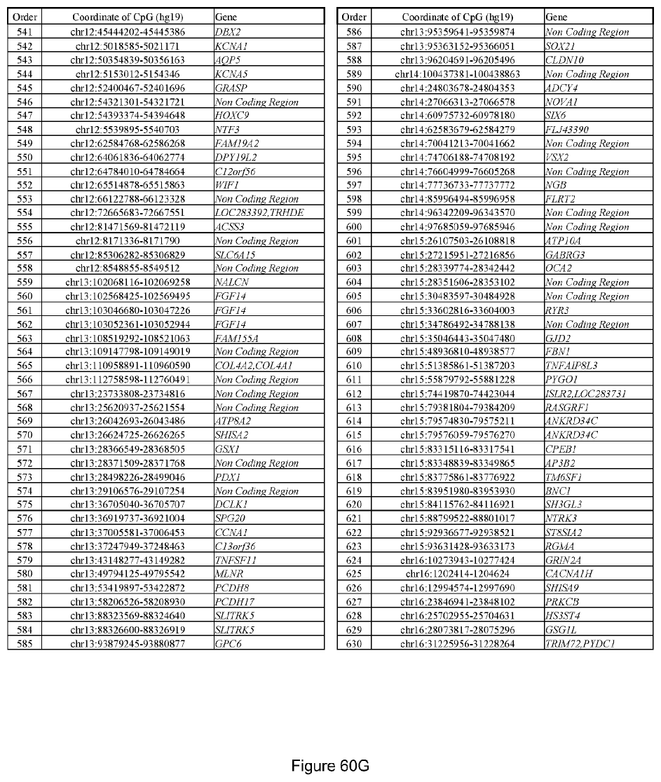

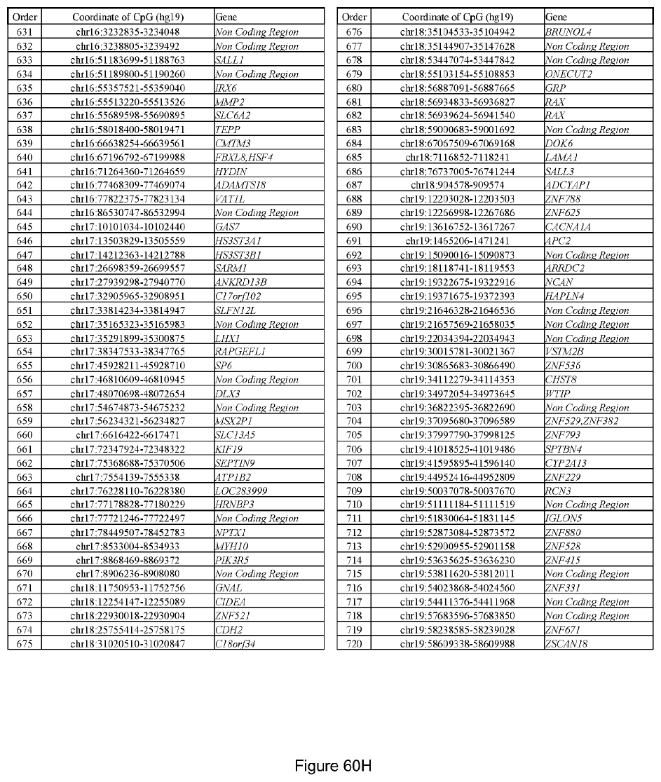

FIG. 59 provides a list of primary CpG sites that are colorectal, stomach, and esophageal cancer and tissue-specific markers, that may be used to identify the presence of colorectal, stomach, and esophageal cancer from cfDNA, DNA within exosomes, or DNA in other protected states (such as within CTCs) within the blood. FIG. 60 provides a list of chromosomal regions or sub-regions within which are primary CpG sites that are colorectal, stomach, and esophageal cancer and tissue-specific markers, that may be used to identify the presence of colorectal, stomach, and esophageal cancer from cfDNA, DNA within exosomes, or DNA in other protected states (such as within CTCs) within the blood. These lists contain preferred primary CpG sites and their flanking sites, as well as alternative markers that are high in CRC, and alternative markers that are low to no-CRC, but high in stomach and/or esophageal cancers. Primer sets for exemplary preferred and alternate methylation markers are listed in Table 53 in the experimental section. A selection of 64 of these markers with average sensitivities of 50% gave the following scores for Group 1: (colorectal=48, stomach=51, esophagus=43), which would translate into the following number of marker equivalents with average sensitivities of 50% (=64×score/50); (colorectal=62 marker equivalents; stomach=65 marker equivalents; esophagus=55 marker equivalents) and thus all were well above the average 36-marker equivalents minimum. The marker equivalents with average sensitivities of 66% gave the following scores (=64×score/66); (colorectal=47 marker equivalents; stomach=50 marker equivalents; esophagus=42 marker equivalents). Thus, all were well above the average 36-marker equivalents minimum.

Group 2 (breast, endometrial, ovarian, cervical, and uterine): Blood-based, breast, endometrial, ovarian, cervical, and uterine cancer-specific microRNA markers that may be used to distinguish group 2 from other groups include, but are not limited to: (mir ID, Gene ID): hsa-mir-1265, MIR1265. This marker was identified through analysis of TCGA microRNA datasets, which may be present in exosomes, tumor-associated vesicles, Argonaute complexes, or other protected states in the blood.

Blood-based breast, endometrial, ovarian, cervical, and uterine cancer-specific exon transcripts that may be used to distinguish group 2 from other groups include, but are not limited to: (mir ID, Gene ID): hsa-mir-1265, MIR1265. This marker was identified through analysis of TCGA microRNA datasets, which may be present in exosomes, tumor-associated vesicles, Argonaute complexes, or other protected states in the blood.

Breast, endometrial, ovarian, cervical, and uterine cancer protein markers that may be used to distinguish group 2 from other groups include, but are not limited to: (Gene Symbol, Chromosome Band, Gene Title, UniProt ID): RSPO2, 8q23.1, R-spondin 2, Q6UXX9; KLC4, 6p21.1, kinesin light chain 4, Q9NSK0; GLRX, 5q14, glutaredoxin (thioltransferase), P35754. These markers may be identified through mRNA sequences, protein expression levels, protein product concentrations, cytokines, or autoantibody to the protein product arising from breast, endometrial, ovarian, cervical, and uterine cancers, which may be identified in the blood, either within exosomes, other protected states, tumor-associated vesicles, or free within the plasma.

Protein markers that can be secreted by breast, endometrial, ovarian, cervical, and uterine cancer into the blood that may be used to distinguish group 2 from other groups include, but are not limited to: (Protein name, UniProt ID); R-spondin-2 (Roof plate-specific spondin-2) (hRspo2), Q6UXX9. A comparative analysis was performed across various TCGA datasets (tumors, normals), followed by an additional bioinformatics filter (Meinken et al., 2012, as described above), which predicts the likelihood that the translated protein is secreted by the cells.

The distribution of mutations in breast, endometrial, ovarian, cervical, and uterine cancer are available in the public COSMIC database, with the most common being: PIK3CA (phosphatidylinositol-4,5-bisphosphate 3-kinase catalytic subunit alpha), and TTN (titin).

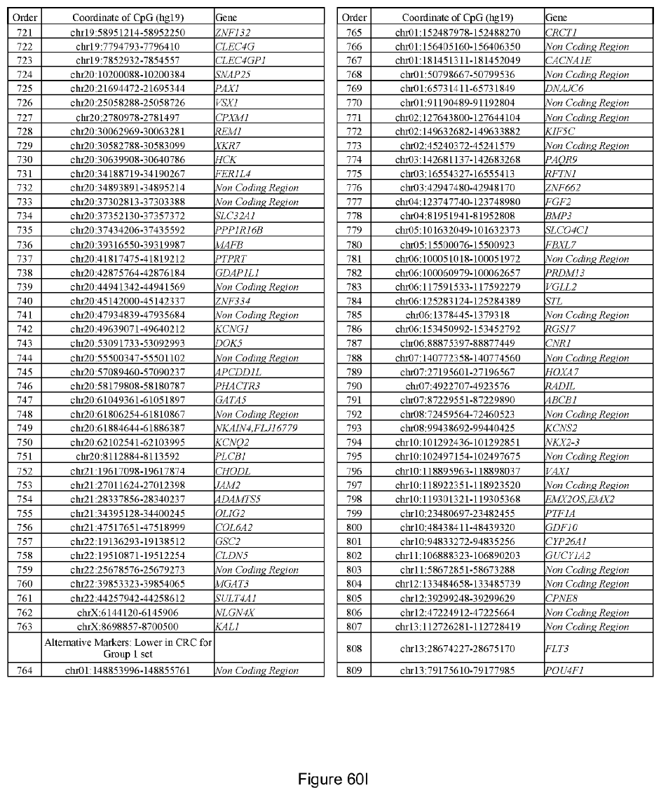

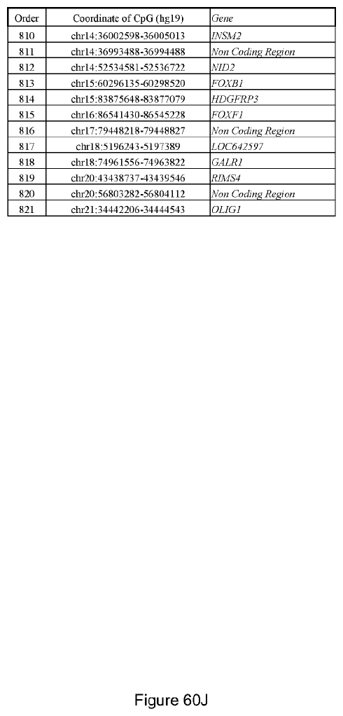

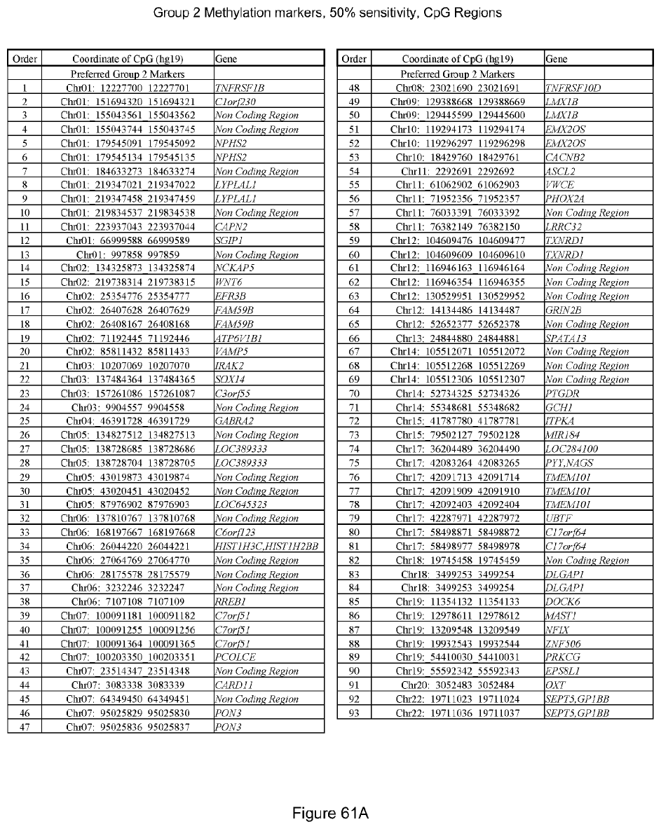

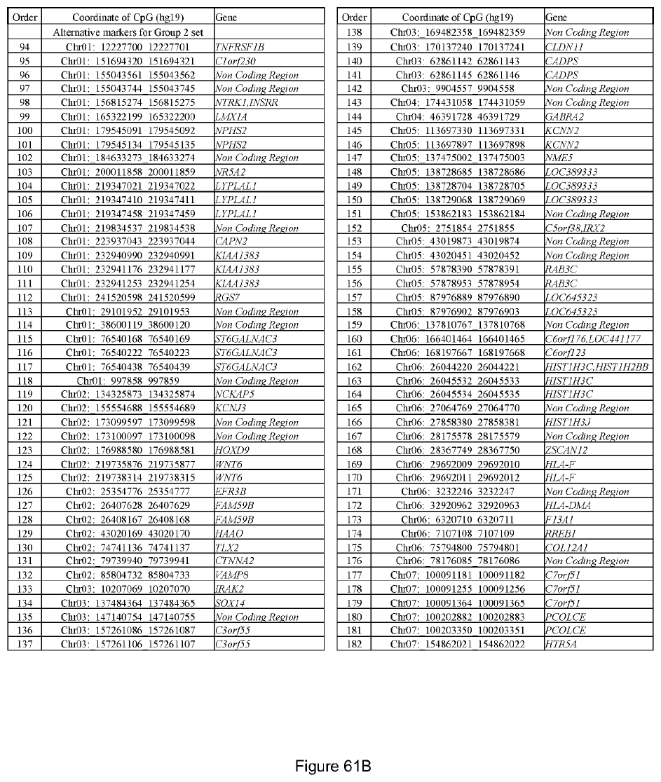

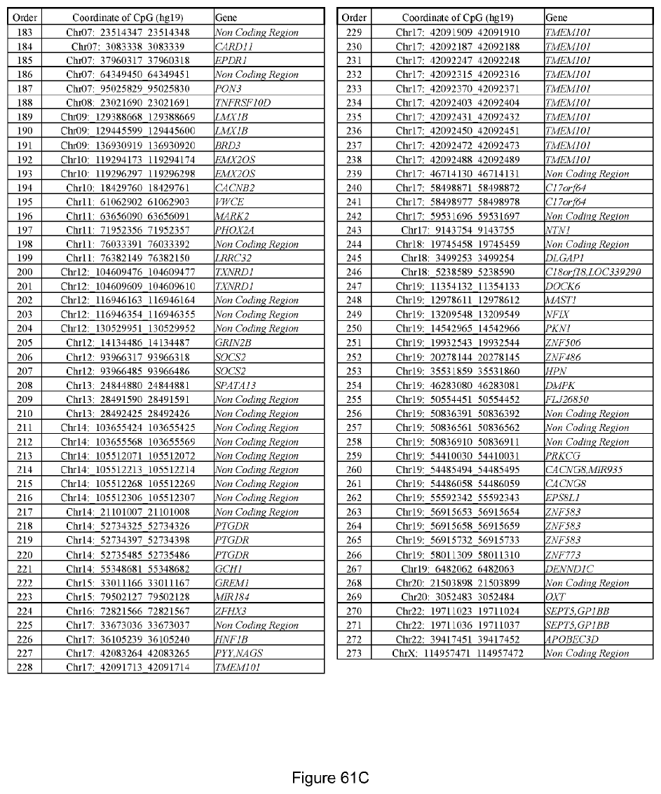

FIG. 61 provides a list of primary CpG sites that are breast, endometrial, ovarian, cervical, and uterine cancer and tissue-specific markers, that may be used to identify the presence of breast, endometrial, ovarian, cervical, and uterine cancer from cfDNA, DNA within exosomes, or DNA in other protected states (such as within CTCs) within the blood. FIG. 62 provides a list of chromosomal regions or sub-regions within which are primary CpG sites that are breast, endometrial, ovarian, cervical, and uterine cancer and tissue-specific markers, that may be used to identify the presence of breast, endometrial, ovarian, cervical, and uterine cancer from cfDNA, DNA within exosomes, or DNA in other protected states (such as within CTCs) within the blood. These lists contain preferred primary CpG sites and their flanking sites, as well as alternative markers that may be used to distinguish breast, endometrial, ovarian, cervical, and uterine cancers. Primer sets for exemplary preferred and alternate methylation markers are listed in Table 54 in the experimental section. A selection of 64 of these markers with average sensitivities of 50% gave the following scores for Group 2: (breast=36, endometrial=49, ovarian=32, cervical=33, uterine=47), which would translate into the following number of marker equivalents with average sensitivities of 50% (=64×score/50); (breast=47 marker equivalents; endometrial=63 marker equivalents; ovarian=41 marker equivalents; cervical=42 marker equivalents; uterine=61 marker equivalents). Thus, all were well above the average 36-marker equivalents minimum. The marker equivalents with average sensitivities of 66% gave the following scores (=64×score/66); (breast=35 marker equivalents; endometrial=48 marker equivalents; ovarian=31 marker equivalents; cervical=32 marker equivalents; uterine=46 marker equivalents). Thus three markers are below and two markers are above the average 36-marker equivalents minimum. However, such scores may be improved by selection of different markers.

Group 3 (lung adenocarcinoma, lung squamous cell carcinoma, and head & neck): Blood-based, lung, head, and neck cancer-specific microRNA markers that may be used to distinguish group 3 from other groups include, but are not limited to: (mir ID, Gene ID): hsa-mir-28, MIR28. This marker was identified through analysis of TCGA microRNA datasets, and may be present in exosomes, tumor-associated vesicles, Argonaute complexes, or other protected states in the blood.

Blood-based lung, head, and neck cancer-specific exon transcripts that may be used to distinguish group 3 from other groups include, but are not limited to: (Exon location, Gene); chr2: chr1:93307721-93309752:−, FAM69A; chr1:93312740-93312916:−, FAM69A; chr1:93316405-93316512:−, FAM69A; chr1:93341853-93342152:−, FAM69A; chr1:93426933-93427079:−, FAM69A; chr7:40221554-40221627:+, C7orf10; chr7:40234539-40234659:+, C7orf10; chr8:22265823-22266009:+, SLC39A14; chr8:22272293-22272415:+, SLC39A14; chr14:39509936-39510091:−, SEC23A; chr14:39511990-39512076:−, SEC23A, and may be enriched in exosomes, tumor-associated vesicles, or other protected states in the blood.

Lung, head, and neck cancer protein encoding markers that may be used to distinguish group 3 from other groups include, but are not limited to: (Gene Symbol, Chromosome Band, Gene Title, UniProt ID): STRN3, 14q13-q21, striatin, calmodulin binding protein 3, Q13033; LRRC17, 7q22.1, leucine rich repeat containing 17, Q8N6Y2; FAM69A, 1p22, family with sequence similarity 69, member A, Q5T7M9; ATF2, 2q32, activating transcription factor 2, P15336; BHMT, 5q14.1, betaine-homocysteine S-methyltransferase, Q93088; ODZ3/TENM3, 4q34.3-q35.1, teneurin transmembrane protein 3, Q9P273; ZFHX4, 8q21.11, zinc finger homeobox 4, Q86UP3. These markers may be identified through mRNA sequences, protein expression levels, protein product concentrations, cytokines, or autoantibody to the protein product arising from lung, head, and neck cancers, which may be identified in the blood, either within exosomes, other protected states, tumor-associated vesicles, or free within the plasma.

Protein markers that can be secreted by lung, head, and neck cancer into the blood may be used to distinguish group 3 from other groups include, but are not limited to: (Protein name, UniProt ID); Leucine-rich repeat-containing protein 17 (p37NB), Q8N6Y2. A comparative analysis was performed across various TCGA datasets (tumors, normals), followed by an additional bioinformatics filter (Meinken et al., “Computational Prediction of Protein Subcellular Locations in Eukaryotes: an Experience Report,” Computational Molecular Biology 2(1):1-7 (2012), which is hereby incorporated by reference in its entirety), which predicts the likelihood that the translated protein is secreted by the cells.

The distribution of mutations in lung, head, and neck cancer are available in the public COSMIC database, with the common being: CSMD3 (CUB and Sushi multiple domains 3), DNAH5 (dynein axonemal heavy chain 5), FAT1 (FAT atypical cadherin 1), FLG (filaggrin), KRAS (Ki-ras2 Kirsten rat sarcoma viral oncogene homolog), LRP1B (LDL receptor related protein 1B), MUC16 (mucin 16, cell surface associated), PCLO (piccolo presynaptic cytomatrix protein), PM-DILI (PKHD1 like 1), RELN (reelin), RYR2 (ryanodine receptor 2), SI (sucrase-isomaltase), SYNE1 (spectrin repeat containing nuclear envelope protein 1), TP53 (tumor protein p53), TTN (titin), USH2A (usherin), and XIRP2 (xin actin binding repeat containing 2).

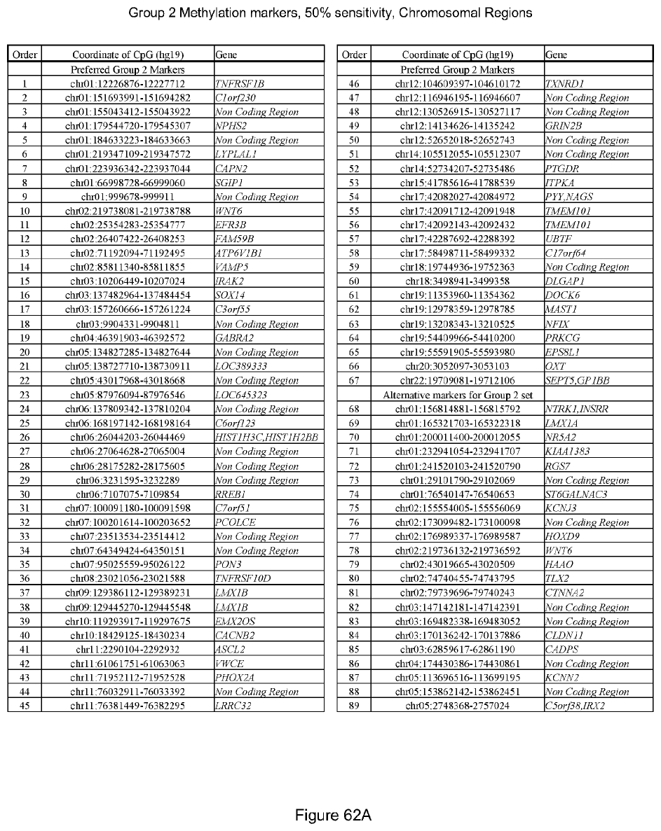

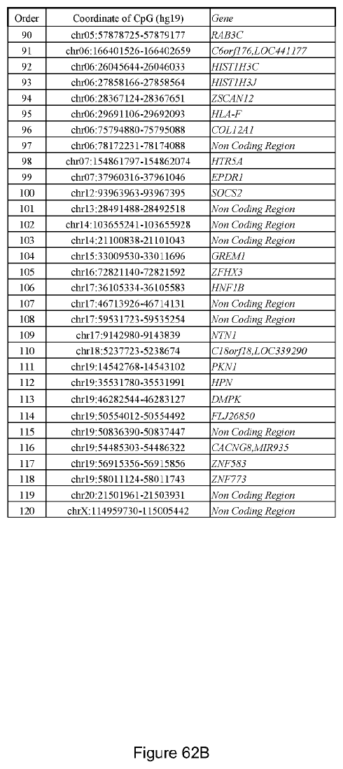

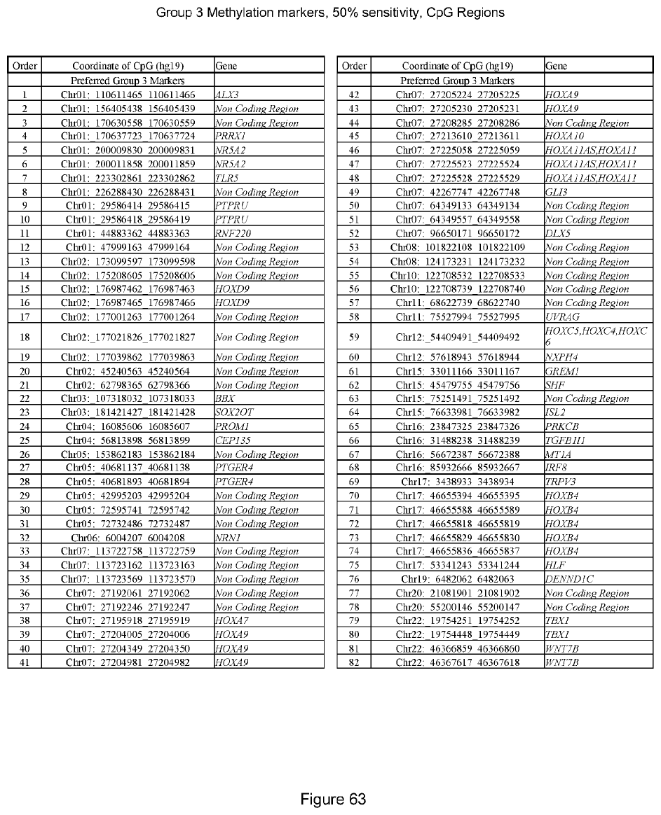

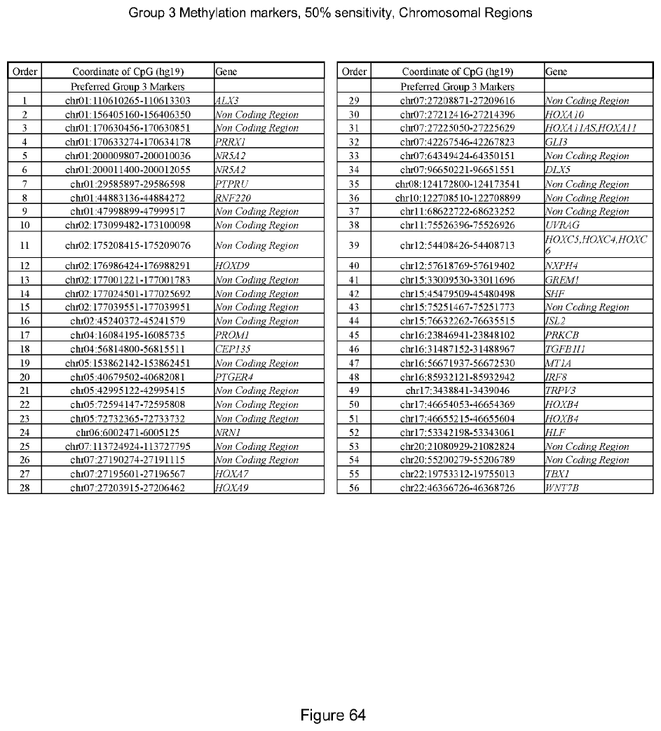

FIG. 63 provides a list of primary CpG sites that are lung, head, and neck cancer and tissue-specific markers, that may be used to identify the presence of lung, head, and neck cancer from cfDNA, DNA within exosomes, or DNA in other protected states (such as within CTCs) within the blood. FIG. 64 provides a list of chromosomal regions or sub-regions within which are primary CpG sites that are lung, head, and neck cancer and tissue-specific markers, that may be used to identify the presence of lung, head, and neck from cfDNA, DNA within exosomes, or DNA in other protected states (such as within CTCs) within the blood. These lists contain preferred primary CpG sites and their flanking sites that may be used to distinguish lung, head, and neck cancers. Primer sets for exemplary methylation markers are listed in Table 55 in the experimental section. A selection of 64 of these markers with average sensitivities of 50% gave the following scores for Group 3: (lung adenocarcinoma=41, lung squamous cell carcinoma=49, head & neck=53), which would translate into the following number of marker equivalents with average sensitivities of 50% (=64×score/50); (lung adenocarcinoma=52 marker equivalents; lung squamous cell carcinoma=62 marker equivalents; head & neck=67 marker equivalents). Thus, all were well above the average 36-marker equivalents minimum. The marker equivalents with average sensitivities of 66% gave the following scores (=64×score/66); (lung adenocarcinoma=40 marker equivalents; lung squamous cell carcinoma=47 marker equivalents; head & neck=51 marker equivalents). Thus all were well above the average 36-marker equivalents minimum.

Group 4 (prostate and bladder): Blood or urine-based, prostate and bladder cancer-specific microRNA markers may be used to distinguish group 4 from other groups include, but are not limited to: (mir ID, Gene ID): hsa-mir-491, MIR491; hsa-mir-1468, MIR1468. These markers were identified through analysis of TCGA microRNA datasets, and may be present in exosomes, tumor-associated vesicles, Argonaute complexes, or other protected states in the blood or urine.

Blood or urine-based, prostate and bladder cancer-specific ncRNA and lncRNA markers that may be used to distinguish group 4 from other groups include, but are not limited to: [Gene ID, Coordinate (GRCh38), ENSEMBL ID]: AC007383.3, chr2:206084605-206086564, ENSG00000227946.1; LINC00324, chr17:8220642-8224043, ENSG00000178977.3. These markers were identified through comparative analysis of various publicly available Affymetrix Exon ST CEL data, which were aligned to GENCODE annotations to generate ncRNA and lncRNA transcriptome datasets. Such lncRNA and ncRNA may be enriched in exosomes or other protected states in the blood or urine.

Blood or urine-based prostate and bladder cancer-specific exon transcripts that may be used to distinguish group 4 from other groups include, but are not limited to: (Exon location, Gene); chr21:45555942-45556055:+, C21orf33 and may be enriched in exosomes, tumor-associated vesicles, or other protected states in the blood or urine.

Prostate and bladder cancer protein markers that may be used to distinguish group 4 from other groups include, but are not limited to: (Gene Symbol, Chromosome Band, Gene Title, UniProt ID): PMM1, 22q13, phosphomannomutase 1, Q92871. This marker may be identified through mRNA sequences, protein expression levels, protein product concentrations, cytokines, or autoantibody to the protein product arising from lung, head, and neck cancers, which may be identified in the blood, either within exosomes, other protected states, tumor-associated vesicles, or free within the plasma, or within the urine.

The distribution of mutations in prostate and bladder cancer are available in the public COSMIC database, with the most common being BAGE2 (BAGE family member 2), DNM1P47 (dynamin 1 pseudogene 47), FRG1BP (region gene 1 family member B, pseudogene), KRAS (Ki-ras2 Kirsten rat sarcoma viral oncogene homolog), RP11-156P1.3, TTN (titin), and TUBB8P7 (tubulin beta 8 class VIII pseudogene 7).

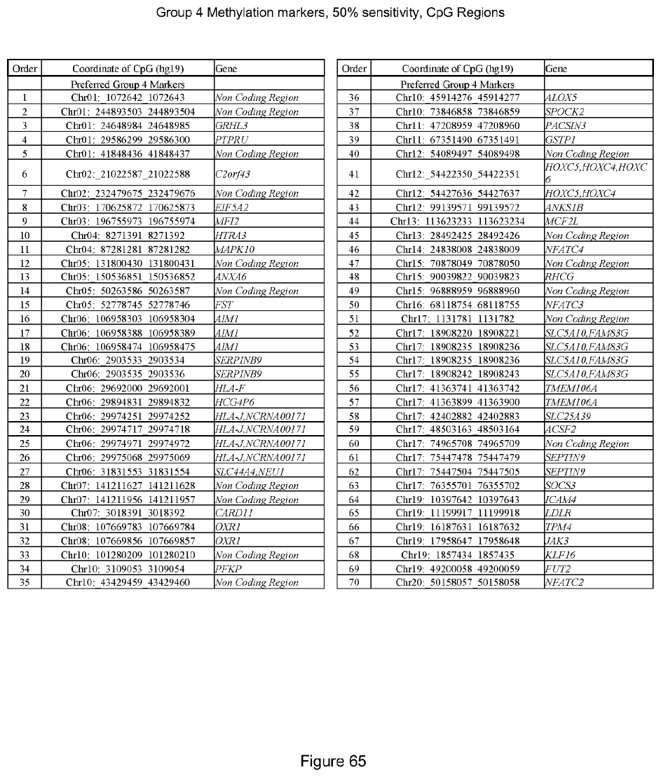

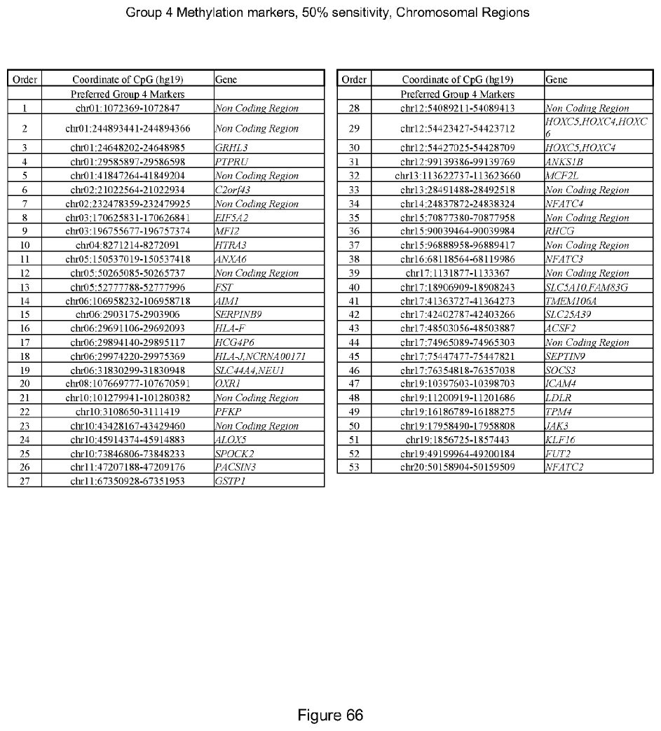

FIG. 65 provides a list of primary CpG sites that are prostate and bladder cancer-specific markers, that may be used to identify the presence of prostate and bladder cancer from cfDNA, DNA within exosomes, or DNA in other protected states (such as within CTCs) within the blood or urine. FIG. 66 provides a list of chromosomal regions or sub-regions within which are primary CpG sites that are prostate and bladder cancer specific markers, that may be used to identify the presence of prostate and bladder from cfDNA, or DNA within exosomes, or DNA in other protected states (such as within CTCs) within the blood or urine. These lists contain preferred primary CpG sites and their flanking sites that may be used to distinguish prostate and bladder cancers. Primer sets for exemplary methylation markers are listed in Table 56 in the experimental section. A selection of 48 of these markers with average sensitivities of 50% gave the following scores for Group 4: (prostate=48, bladder=22), which would translate into the following number of marker equivalents with average sensitivities of 50% (=48×score/50); (prostate=46 marker equivalents; bladder=21 marker equivalents). Thus, bladder was below the average 36-marker equivalents minimum. Likewise, the marker equivalents with average sensitivities of 66% gave the following scores (=48×score/60); (prostate=35 marker equivalents; bladder=16 marker equivalents). Thus, bladder was well below the average 36-marker equivalents minimum. However, a different selection of markers, for example by increasing from 48 to 64 markers and including markers that were positive for both prostate and bladder, would rectify this situation. The markers were limited to those that were not methylated in normal prostate, bladder, or kidney tissue to minimize false-positive results from urine samples.

Group 5 (liver, pancreatic and gall-bladder): Blood-based, liver, pancreatic and gall-bladder cancer-specific microRNA markers that may be used to distinguish group 5 from other groups include, but are not limited to: (mir ID, Gene ID): hsa-mir-132, MIR132. This marker was identified through analysis of TCGA microRNA datasets, which may be present in exosomes, tumor-associated vesicles, Argonaute complexes, or other protected states in the blood.

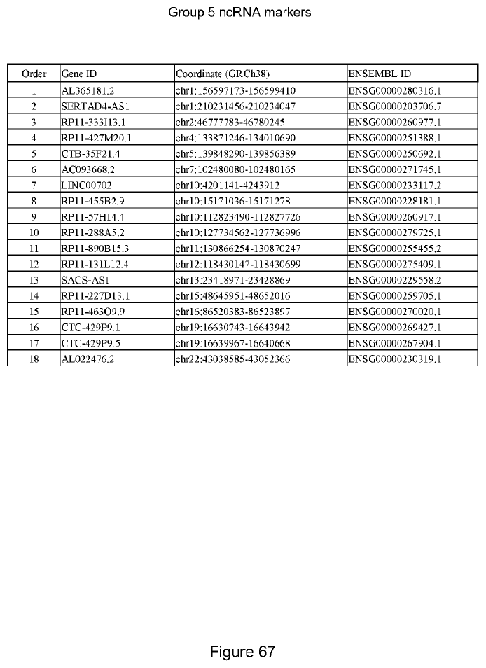

FIG. 67 provides a list of blood-based, liver, pancreatic and gall-bladder cancer-specific ncRNA and lncRNA markers derived through comparative analysis of various publicly available Affymetrix Exon ST CEL data, which were aligned to GENCODE annotations to generate ncRNA and lncRNA transcriptome datasets. Such lncRNA and ncRNA may be enriched in exosomes or other protected states in the blood.

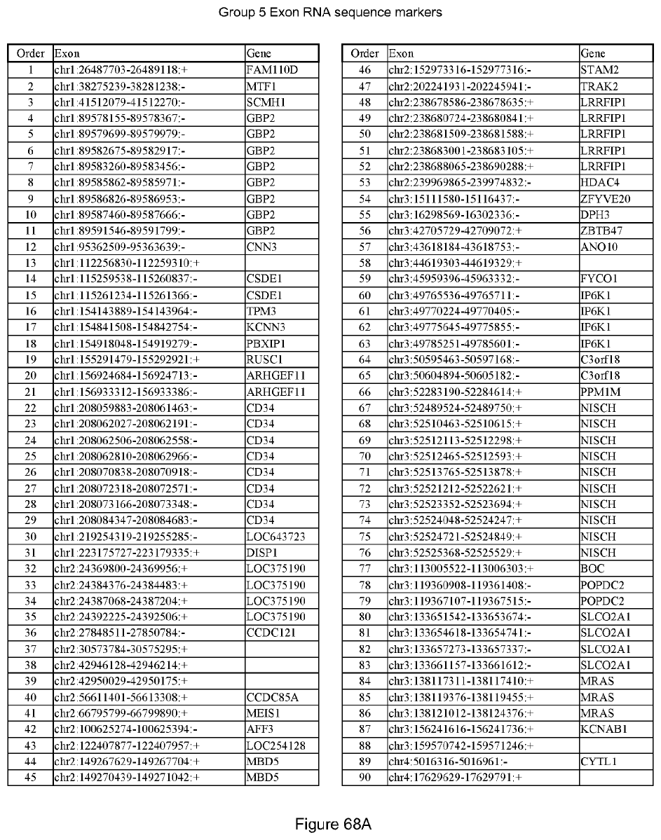

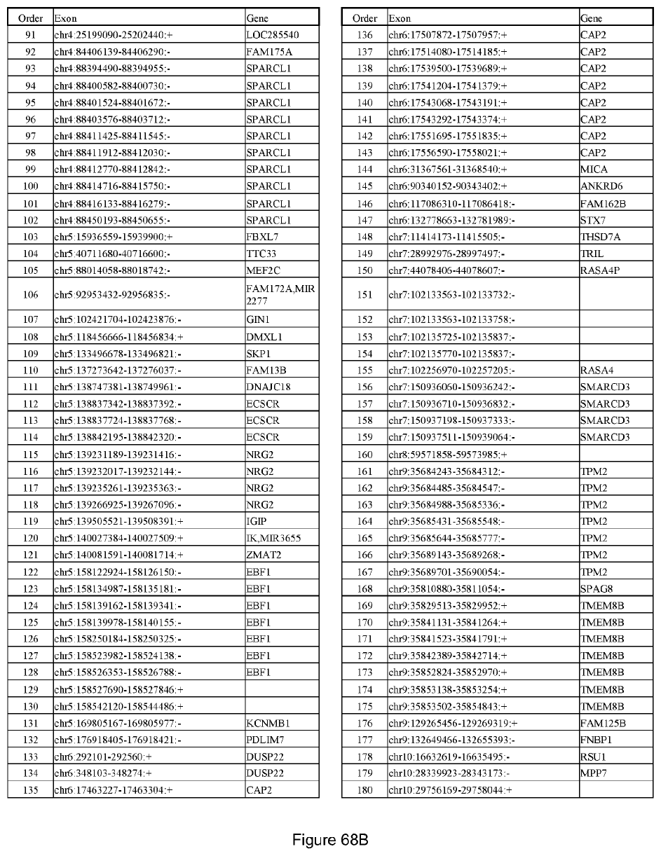

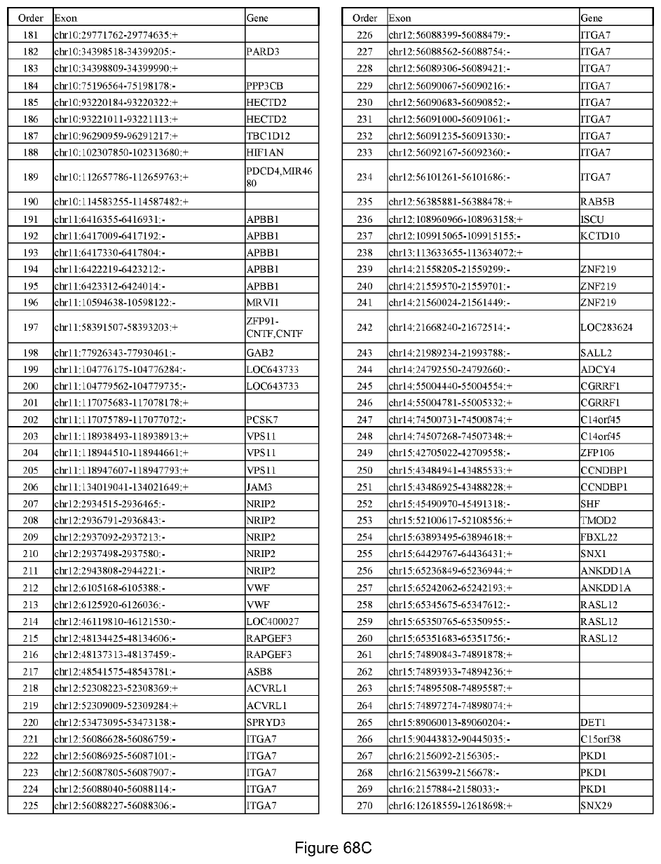

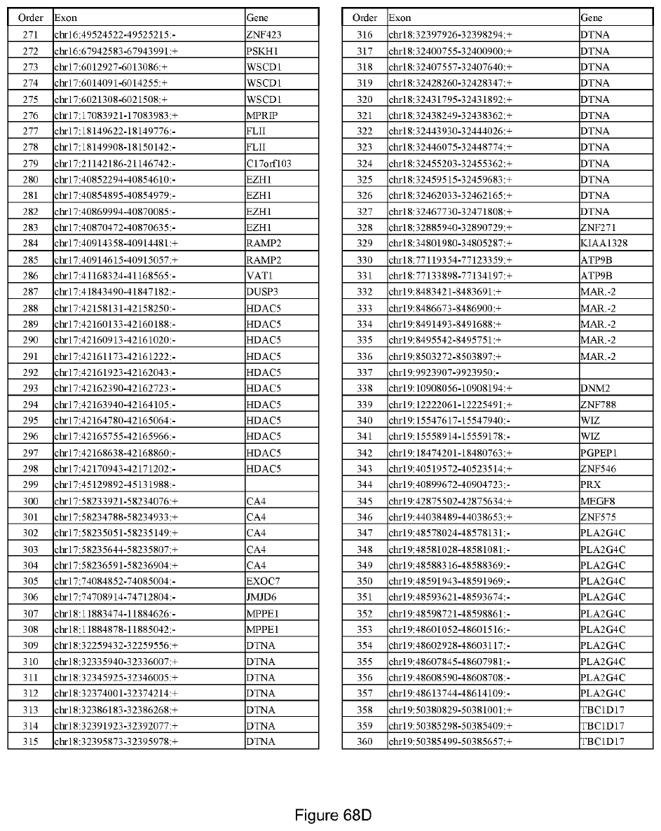

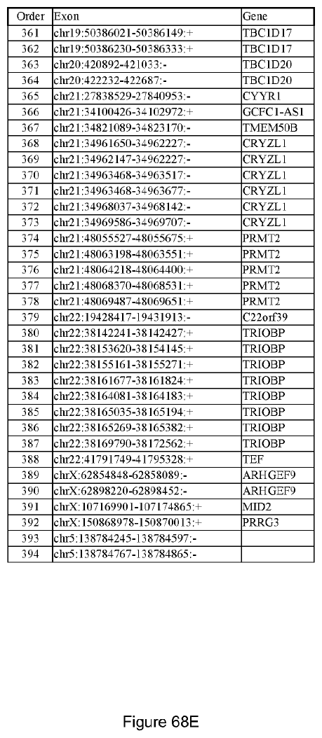

In addition, FIG. 68 provides a list of blood-based liver, pancreatic and gall-bladder cancer-specific exon transcripts that may be enriched in exosomes, tumor-associated vesicles, or other protected states in the blood.

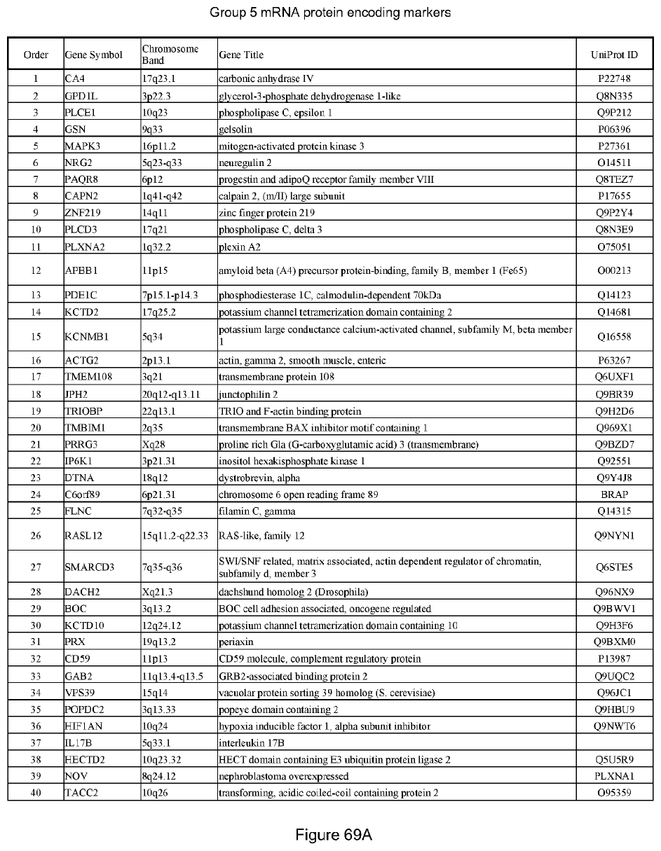

FIG. 69 provides a list of liver, pancreatic and gall-bladder cancer protein markers, identified through mRNA sequences, protein expression levels, protein product concentrations, cytokines, or autoantibody to the protein product arising from liver, pancreatic and gall-bladder cancers, which may be identified in the blood, either within exosomes, other protected states, tumor-associated vesicles, or free within the plasma.

Protein markers that can be secreted by liver, pancreatic and gall-bladder cancer into the blood that may be used to distinguish group 5 from other groups include, but are not limited to: (Protein name, UniProt ID); Gelsolin (AGEL) (Actin-depolymerizing factor) (ADF) (Brevin), P06396; Pro-neuregulin-2, O14511; CD59 glycoprotein (1F5 antigen) (20 kDa homologous restriction factor) (HRF-20) (HRF20) (MAC-inhibitory protein) (MAC-IP) (MEM43 antigen) (Membrane attack complex inhibition factor) (MACIF) (Membrane inhibitor of reactive lysis) (MIRL) (Protectin) (CD antigen CD59), P13987; Divergent protein kinase domain 2B (Deleted in autism-related protein 1), Q9H7Y0. A comparative analysis was performed across various TCGA datasets (tumors, normals), followed by an additional bioinformatics filter (Meinken et al., “Computational Prediction of Protein Subcellular Locations in Eukaryotes: an Experience Report,” Computational Molecular Biology 2(1):1-7 (2012), which is hereby incorporated by reference in its entirety), which predicts the likelihood that the translated protein is secreted by the cells.

The distribution of mutations in liver, pancreatic and gall-bladder cancer are available in the public COSMIC database, with the most common being: KRAS (Ki-ras2 Kirsten rat sarcoma viral oncogene homolog), MUC16 (mucin 16, cell surface associated), MUC4 (mucin 4, cell surface associated), TP53 (tumor protein p53), and TTN (titin).

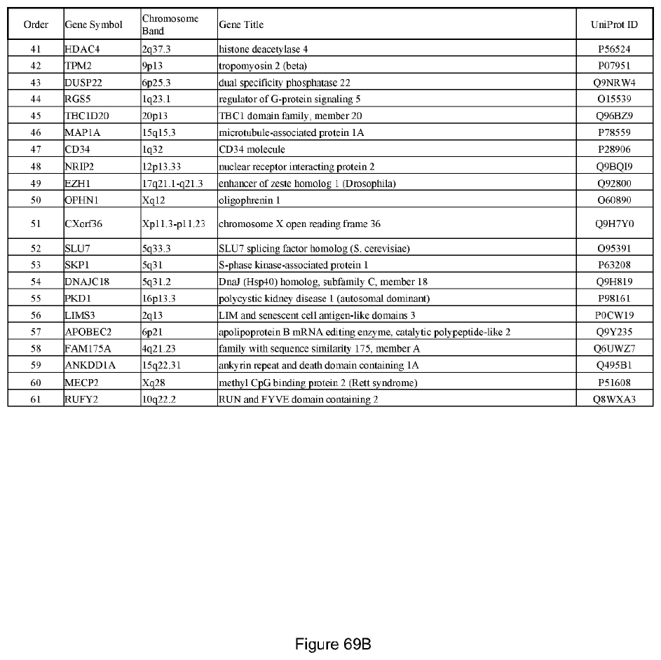

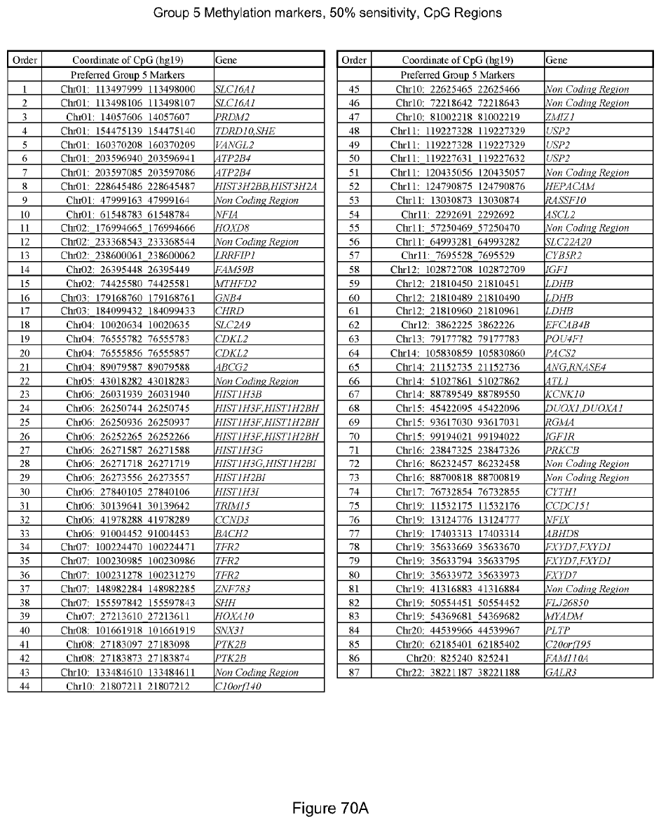

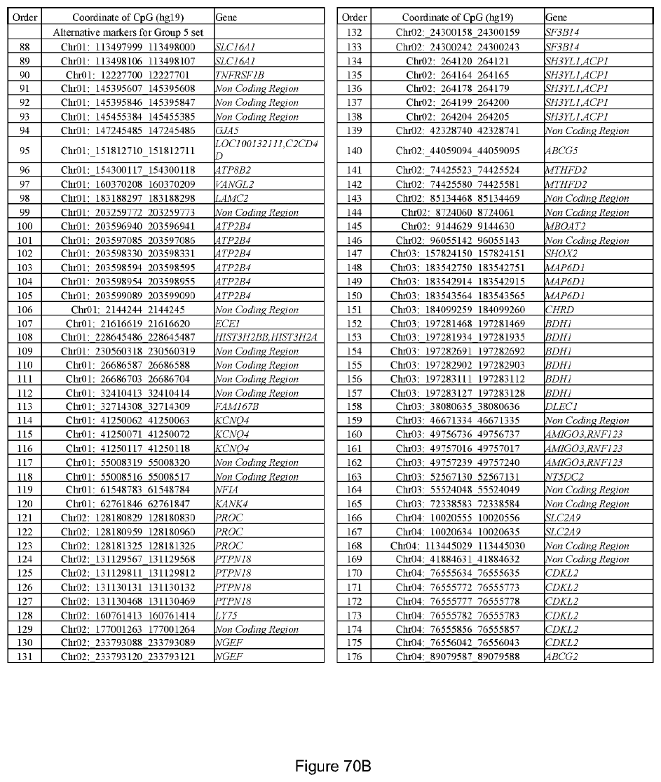

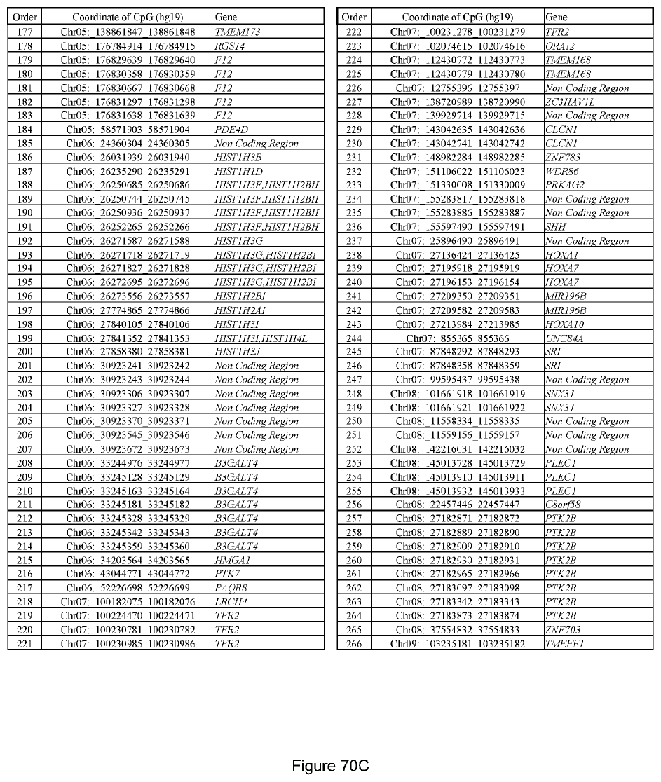

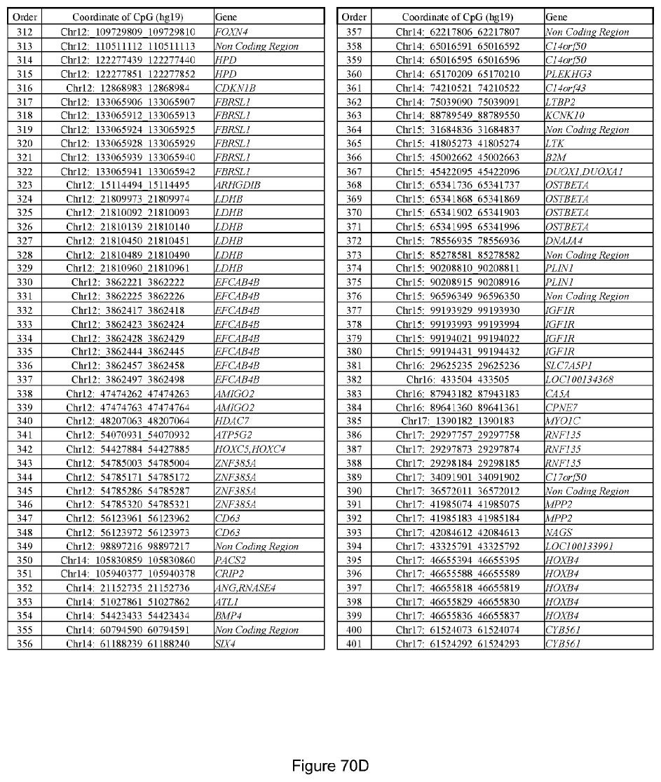

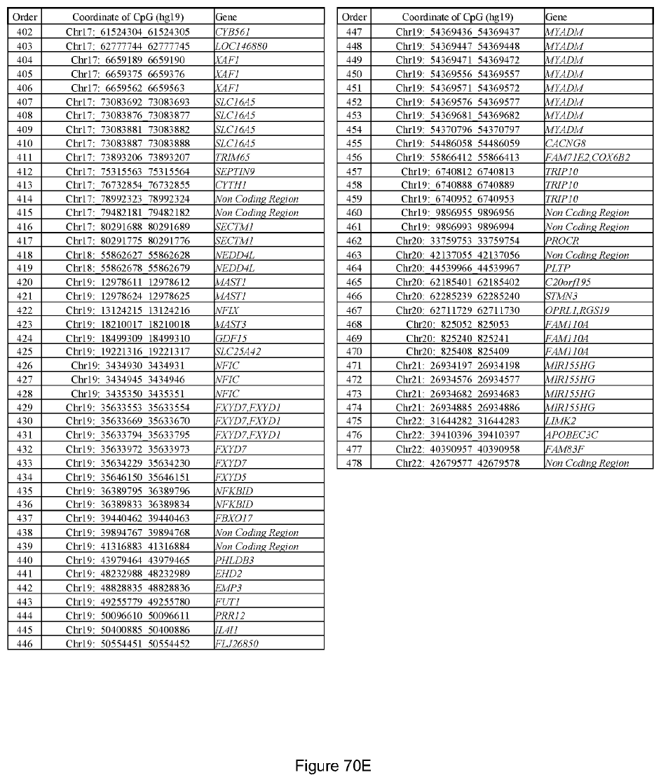

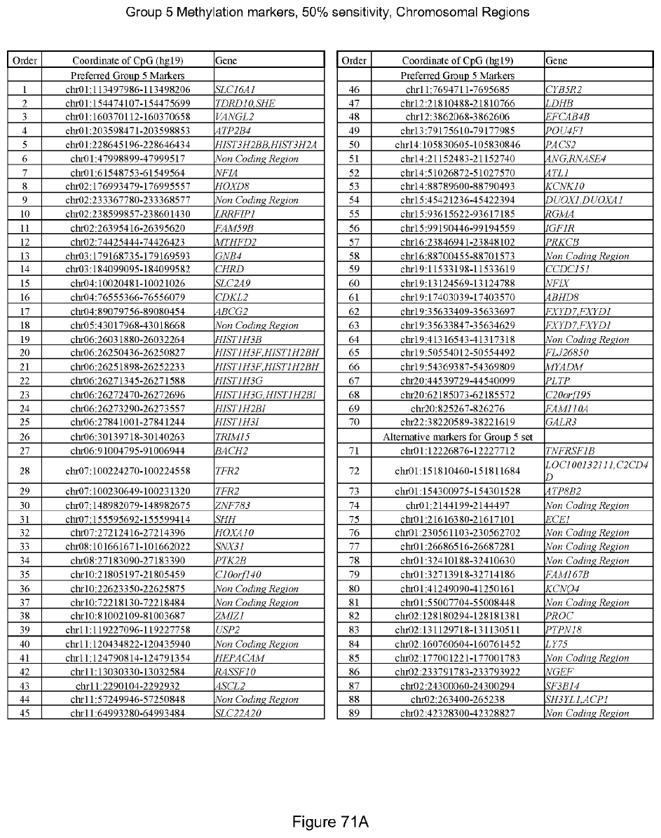

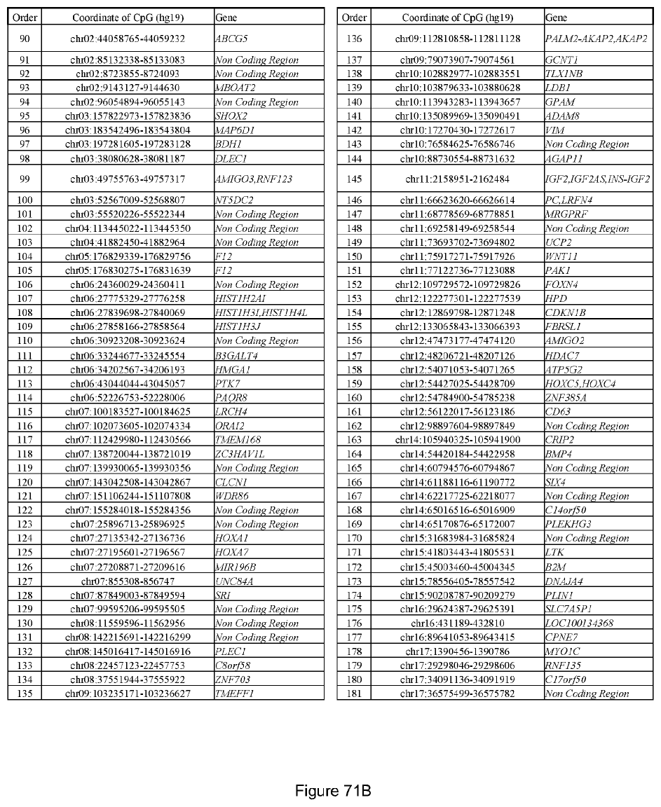

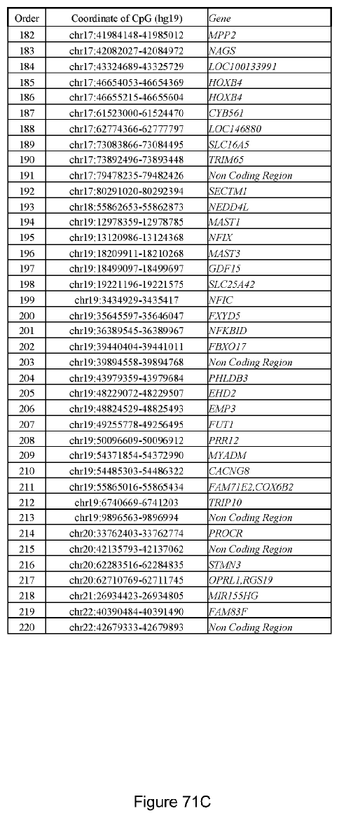

FIG. 70 provides a list of primary CpG sites that are liver, pancreatic and gall-bladder cancer and tissue-specific markers, that may be used to identify the presence of lung, head, and neck cancer from cfDNA, DNA within exosomes, or DNA in other protected states (such as within CTCs) within the blood. FIG. 71 provides a list of chromosomal regions or sub-regions within which are primary CpG sites that are liver, pancreatic and gall-bladder cancer and tissue-specific markers, that may be used to identify the presence of liver, pancreatic and gall-bladder from cfDNA, DNA within exosomes, or DNA in other protected states (such as within CTCs) within the blood. These lists contain preferred primary CpG sites and their flanking sites, as well as alternative markers that may be used to distinguish liver, pancreatic and gall-bladder cancers. Primer sets for exemplary preferred and alternate methylation markers are listed in Table 57 in the experimental section. A selection of 64 of these markers with average sensitivities of 50% gave the following scores for Group 5: (liver=57, pancreatic=30, gall bladder=60), which would translate into the following number of marker equivalents with average sensitivities of 50% (=64×score/50); (liver=73 marker equivalents; pancreatic=38 marker equivalents; gall bladder=77 marker equivalents). Thus, all were above the average 36-marker equivalents minimum. The marker equivalents with average sensitivities of 66% gave the following scores (=64×score/66); (liver=56 marker equivalents; pancreatic=29 marker equivalents; gall bladder=58 marker equivalents). Thus, liver and gall bladder were above the average 36-marker equivalents minimum, while pancreatic was below.

Consider the first strategy using the 96 pan-oncology markers of detecting early colorectal cancer (FIG. 1C). The calculations are done with the anticipation of an average of 150 methylated molecules per positive marker in the blood. As described supra, for the example of colorectal cancer, in particular the cases of microsatellite stable tumors (MSS) where the mutation load is low, for these calculations when relying on NGS sequencing alone (assuming 150 molecules with one mutation in the blood), an estimated 78% of early colorectal cancer would be missed. Again, to put these number in perspective, in the U.S., about 135,000 new cases of colorectal cancer were diagnosed in 2018, of which about 60% is late cancer (i.e. Stage III & IV). About 107 million individuals in the U.S. are over the age of 50 and should be tested for colorectal cancer. While it cannot be predicted how many individuals have a hidden cancer (i.e. Stage I) within them, who are non-compliant to testing, for the purposes of this calculation, assume that the average late cancer was once the average early cancer, and thus individuals with Stage I cancer would be about 40,500 individuals. Assuming individual marker false-positive rates of 3%, and with the first step using 96 markers (48 markers for CRC) with average sensitivities of 50%, requiring a minimum of 5 markers positive, then with an overall specificity of 95.8%, the first step would identify 4,494,000 individuals (out of 107,000,000 total adults over 50 in the U.S.) which would include at 71.6% sensitivity or about 28,998 individuals with Stage I colorectal cancer (out of 40,500 individuals with Stage I cancer). However, those 4,494,000 presumptive positive individuals would be evaluated in a second step of 64 markers (48 markers for CRC) with average sensitivities of 50%, requiring a minimum of 5 markers positive, then the two-step test would identify 71.6%×71.6%=51.2%=20,762 individuals (out of 40,500 individuals with Stage I cancer) with colorectal cancer. With a specificity of 95.8%, the second test would also generate 4,494,000×4.2%=188,748 false-positives. The positive predictive value of such a test would be 20,762/(188,748+20,762)=9.9%, in other words, 1 in 10 individuals who tested positive would actually have Stage I colorectal cancer. In reality, one would need to also include the success for identifying Stage 2 and higher cancers. In expanding this example, the calculations are done with the anticipation that Stage I CRC has an average of 150 methylated molecules per positive marker in the blood, Stage II CRC has an average of 200 methylated molecules per positive marker, and the higher stages (III & IV) have at least an average of 300 methylated molecules per positive marker, and the higher stages. Also, to be consistent with the idea that as the test is used repeatedly, more of early and less of late CRC will be detected, than an estimate of 40,500 individuals with Stage I cancer, 40,500 individuals with Stage II cancer, and the remaining 54,000 individuals have late-stage cancer=135,000 total individuals with colorectal cancer identified per year in the U.S. The above calculation already provided the false-positive rate for the early cancer. For Stage II cancer, 90.1% would be identified in the first step, of which 90.1%×90.1%=81.0%=32,877 individuals with Stage II cancer would be verified in the second step. For Stage III and IV cancer, 99.3% would be identified in the first step, of which 99.3%×99.3%=98.6%=53,246 individuals with late cancer would be identified. This brings the total identified at 20,762+32,877+53,246=106,885 individuals out of 135,000 with colorectal cancer, for an overall sensitivity of 79%. Overall, the positive predictive value of such a test would be 106,885/(188,748+106,885)=36.1%, in other words, 1 in 3 individuals who tested positive would actually have colorectal cancer, and this test would identify 53,639/81,000 or 66% of those individuals with early cancer, compared with the current rate of 40%.

How would these results vary for using this strategy (FIG. 1C) for detection of early colorectal cancer using 50% average marker sensitivities, with the anticipation of Stage I CRC having an average of 200 methylated molecules per positive marker in the blood, Stage II CRC having an average of 240 methylated molecules per positive marker, and the higher stages (III & IV) having at least an average of 300 methylated molecules per positive marker?

Assuming individual marker false-positive rates of 3%, the first step using 96 markers (48 markers for CRC) with average sensitivities of 50%, requiring a minimum of 5 markers positive, and an overall specificity of 95.8%, the first step would identify 4,494,000 individuals (out of 107,000,000 total adults over 50 in the U.S.). This would include, at 90.1% sensitivity, or about 36,490 individuals with Stage I colorectal cancer (out of 40,500 individuals with Stage I cancer). However, those 4,494,000 presumptive positive individuals would be evaluated in a second step of 64 markers (48 markers for CRC) with average sensitivities of 50%, requiring a minimum of 5 markers positive. The two-step test would identify 90.1%×90.1%=81.2%=32,877 individuals (out of 40,500 individuals with Stage I cancer) with colorectal cancer. With a specificity of 95.8%, the second test would also generate 4,494,000×4.2%=188,748 false-positives. The positive predictive value of such a test would be 32,877/(188,748+32,877)=14.8%. In other words, 1 in 6.7 individuals who tested positive would actually have Stage I colorectal cancer. In reality, one would need to also include the success for identifying Stage 2 and higher cancers. To be consistent with the idea that, as the test is used repeatedly, more of early and less of late CRC will be detected, an estimated 40,500 individuals with Stage I cancer, 40,500 individuals with Stage II cancer, and 54,000 individuals with late-stage cancer (135,000 total individuals with colorectal cancer) would be identified per year in the U.S. The above calculation already provided the false-positive rate for the early cancer. For Stage II cancer, 90.1% would be identified in the first step, of which 97.2%×97.2%=94.5%=38,263 individuals with Stage II cancer would be verified in the second step. For Stage III and IV cancer, 99.3% would be identified in the first step, of which 99.3%×99.3%=98.6%=53,246 individuals with late cancer would be identified. This brings the total identified to 32,877+38,263+53,246=124,386 individuals out of 135,000 with colorectal cancer, for an overall sensitivity of 92.1%. Overall, the positive predictive value of such a test would be 124,386/(188,748+124,386)=39.7%. In other words, 1 in 2.5 individuals who tested positive would actually have colorectal cancer, and this test would identify 71,104/81,000 or 87.7% of those individuals with early cancer, compared with the current rate of 40%.

How would these results vary when using this strategy (FIG. 1C) for detection of early ovarian cancer, with the anticipation of an average of 150 methylated molecules per positive marker in the blood? When relying on NGS sequencing alone (assuming 150 molecules with one mutation in the blood), an estimated 78% of early ovarian cancer would be missed. Again, to put these number in perspective, in the U.S., about 22,000 new cases of ovarian cancer were diagnosed in 2018, of which about 85% was late cancer (i.e. Stage III & IV). About 54 million women in the U.S. are between the ages of 50 and 79 and should be tested for ovarian cancer. While it cannot be predicted how many individuals have a hidden cancer (i.e. Stage I), for the purposes of this calculation, assume that the stages are evenly divided. Thus, the number of individuals with Stage I ovarian cancer would be about 5,500 individuals. Assuming individual marker false-positive rates of 3%, the first step using 96 markers (36 markers for ovarian) with average sensitivities of 50%, requiring a minimum of 5 markers positive, and an overall specificity of 99.1%, the first step would identify 486,000 individuals (out of 54,000,000 total women ages 50-79 in the U.S.) with ovarian cancer. This would include, at 46.8% sensitivity, or about 2,574 individuals with Stage I ovarian cancer (out of 5,500 individuals with Stage I ovarian cancer). However, those 486,000 presumptive positive individuals would be evaluated in a second step of 64 markers (36 markers for ovarian cancer) with average sensitivities of 50%, requiring a minimum of 5 markers positive. The two-step test would identify 46.8%×46.8%=21.9%=1,204 individuals (out of 5,500 individuals with Stage I ovarian cancer) with ovarian cancer. With a specificity of 99.1%, the second test would also generate 486,000×0.9%=4,374 false-positives. The positive predictive value of such a test would be 1,204/(4,374+1,204)=21.6%. In other words, 1 in 4.6 individuals who tested positive would actually have Stage I ovarian cancer. In reality, one would need to also include the success for identifying Stage 2 and higher ovarian cancers. In expanding this example, the calculations are done with the anticipation that Stage I ovarian cancer has an average of 150 methylated molecules per positive marker in the blood, Stage II ovarian cancer has an average of 200 methylated molecules per positive marker, and the higher stages (III & IV) have at least an average of 300 methylated molecules per positive marker. To be consistent with the idea that, as the test is used repeatedly, more cancer will be detected and all four stages are at 5,500, then 5,500×4=22,000 total individuals with ovarian cancer would be identified per year in the U.S. The above calculation already provided the false-positive rate for the early cancer. For Stage II cancer, 71.5% would be identified in the first step, of which 71.5%×71.5%=51.1%=2,810 individuals with Stage II ovarian cancer would be verified in the second step. For Stage III and IV ovarian cancer, 94.5% would be identified in the first step, of which 94.5%×94.5%=89.3%=9,823 individuals with late ovarian cancer would be identified. This brings the total identified at 1,204+2,810+9,823=13,837 individuals out of 22,000 with ovarian cancer, for an overall sensitivity of 62.9%. Overall, the positive predictive value of such a test would be 13,837/(13,837+4,374)=76.0%. In other words, 3 in 4 women who tested positive would actually have ovarian cancer, and this test would identify 4,014/11,000, or 36.5%, of those individuals with early cancer, compared with the current rate of 15%.

How would these results vary for using this strategy (FIG. 1C) for detection of early ovarian cancer using 50% average marker sensitivities, with the anticipation that Stage I ovarian cancer has an average of 200 methylated molecules per positive marker in the blood, Stage II ovarian cancer has an average of 240 methylated molecules per positive marker, and the higher stages (III & IV) have at least an average of 300 methylated molecules per positive marker?

Assuming individual marker false-positive rates of 3%, the first step using 96 markers (36 markers for ovarian) with average sensitivities of 50%, and requiring a minimum of 5 markers positive, with an overall specificity of 99.1%, the first step would identify 486,000 individuals (out of 54,000,000 total women ages 50-79 in the U.S.) with ovarian cancer. This would include at, 71.5% sensitivity, about 3,932 individuals with Stage I ovarian cancer (out of 5,500 individuals with Stage I ovarian cancer). However, those 486,000 presumptive positive individuals would be evaluated in a second step of 64 markers (36 markers for ovarian cancer) with average sensitivities of 50%, requiring a minimum of 5 markers positive. The two-step test would identify 71.5%×71.5%=51.1%=2,810 individuals (out of 5,500 individuals with Stage I ovarian cancer) with ovarian cancer. With a specificity of 99.1%, the second test would also generate 486,000×0.9%=4,374 false-positives. The positive predictive value of such a test would be 2,810/(4,374+2,810)=39.1%. In other words, 1 in 2.5 individuals who tested positive would actually have Stage I ovarian cancer. In reality, one would need to also include the success for identifying Stage 2 and higher ovarian cancers. As the test is used repeatedly, assume all four stages are at 5,500, and, therefore, 5,500×4=22,000 total individuals with ovarian cancer would be identified per year in the U.S. The above calculation already provided the false-positive rate for the early cancer. For Stage II cancer, 84.4% would be identified in the first step, of which 84.4%×84.4%=71.2%=3,916 individuals with Stage II ovarian cancer would be verified in the second step. For Stage III and IV ovarian cancer, 94.5% would be identified in the first step, of which 94.5%×94.5%=89.3%=9,823 individuals with late ovarian cancer would be identified. This brings the total identified to 2,810+3,916+9,823=16,549 individuals out of 22,000 with ovarian cancer, for an overall sensitivity of 75.2%. Overall, the positive predictive value of such a test would be 16,549/(16,549+4,374)=79.0%. In other words, 4 in 5 women who tested positive would actually have ovarian cancer. This test would identify 6,006/11,000 or 54.6% of those individuals with early cancer, compared with the current rate of 15%.

The above calculations worked under the assumption of limiting at least one set of markers to an average of 50% sensitivity. How would the results improve if the average of 50% sensitivity was improved to 66% sensitivity?

Consider the strategy of using the 96 pan-oncology markers for detecting early colorectal cancer (FIG. 1D). The calculations are done with the anticipation of an average of 150 methylated molecules per positive marker in the blood. As described supra, assume that the average late cancer was once the average early cancer, and thus individuals with Stage I cancer would be about 40,500 individuals. Assuming individual marker false-positive rates of 3%, the first step using 96 markers (48 markers for CRC) with average sensitivities of 66%, requiring a minimum of 5 markers positive, and an overall specificity of 95.8%, the first step would identify 4,494,000 individuals (out of 107,000,000 total adults over 50 in the U.S.) with Stage I colorectal cancer. This would include, at 90.0% sensitivity, about 36,450 individuals with Stage I colorectal cancer (out of 40,500 individuals with Stage I cancer). However, those 4,494,000 presumptive positive individuals would be evaluated in a second step of 64 markers (48 markers for CRC) with average sensitivities of 66%, and requiring a minimum of 5 markers positive. The two-step test would identify 90.0%×90.0%=89.0%=32,805 individuals (out of 40,500 individuals with Stage I cancer) with colorectal cancer. With a specificity of 95.8%, the second test would also generate 4,494,000×4.2%=188,748 false-positives. The positive predictive value of such a test would be 32,805/(188,748+32,805)=14.8%. In other words, 1 in 7 individuals who tested positive would actually have Stage I colorectal cancer. In reality, one would need to also include the success for identifying Stage 2 and higher cancers. In expanding this example, the calculations are done with the anticipation that Stage I CRC has an average of 150 methylated molecules per positive marker in the blood, Stage II CRC has an average of 200 methylated molecules per positive marker, and the higher stages (III & IV) have at least an average of 300 methylated molecules per positive marker. Also, to be consistent with the idea that as the test is used repeatedly, more of early and less of late CRC will be detected, an estimate of 40,500 individuals would be identified with Stage I cancer, 40,500 individuals would be identified with Stage II cancer, and the remaining 54,000 individuals would be identified with late-stage cancer. This equals 135,000 total individuals with colorectal cancer identified per year in the U.S. The above calculation already provided the false-positive rate for the early cancer. For Stage II cancer, 98.0% would be identified in the first step, of which 98.0%×98.0%=96.0%=38,896 individuals with Stage II cancer would be verified in the second step. For Stage III and IV cancer, 99.6% would be identified in the first step, of which 99.6%×99.6%=99.2%=53,568 individuals with late cancer would be identified. This brings the total identified to 32,805+38,896+53,568=125,269 individuals out of 135,000 with colorectal cancer, for an overall sensitivity of 92.7%. Overall, the positive predictive value of such a test would be 125,269/(188,748+125,269)=39.9%. In other words, 1 in 2.5 individuals who tested positive would actually have colorectal cancer. This test would identify 71,701/81,000, or 88%, of those individuals with early cancer, compared with the current rate of 40%.

How would these results vary for using this strategy (FIG. 1D) for detection of early colorectal cancer using 66% average marker sensitivities, with the anticipation of Stage I CRC having an average of 200 methylated molecules per positive marker in the blood, Stage II CRC having an average of 240 methylated molecules per positive marker in the blood, and the higher stages (III & IV) having at least an average of 300 methylated molecules per positive marker in the blood?

Assuming individual marker false-positive rates of 3%, the first step using 96 markers (48 markers for CRC) with average sensitivities of 66%, and requiring a minimum of 5 markers positive, then, with an overall specificity of 95.8%, the first step would identify 4,494,000 individuals with colorectal cancer (out of 107,000,000 total adults over 50 in the U.S.). This would include, at 98.0% sensitivity, about 39,690 individuals with Stage I colorectal cancer (out of 40,500 individuals with Stage I cancer). However, those 4,494,000 presumptive positive individuals would be evaluated in a second step of 64 markers (48 markers for CRC) with average sensitivities of 66%, requiring a minimum of 5 markers positive. The two-step test would identify 98.0%×98.0%=96.0%=38,896 individuals (out of 40,500 individuals with Stage I cancer) with colorectal cancer. With a specificity of 95.8%, the second test would also generate 4,494,000×4.2%=188,748 false-positives. The positive predictive value of such a test would be 38,896/(188,748+38,896)=17.81%. In other words, 1 in 6 individuals who tested positive would actually have Stage I colorectal cancer. In reality, one would need to also include the success for identifying Stage 2 and higher cancers. To be consistent with the idea that, as the test is used repeatedly, more of early and less of late CRC will be detected, an estimate of 40,500 individuals with Stage I cancer would be identified, 40,500 individuals with Stage II cancer would be identified, and the remaining 54,000 individuals would be identified as having late-stage cancer (i.e., 135,000 total individuals with colorectal cancer identified per year in the U.S.). The above calculation already provided the false-positive rate for the early cancer. For Stage II cancer, 98.0% would be identified in the first step, of which 99.6%×99.6%=99.2%=40,176 individuals with Stage II cancer would be verified in the second step. For Stage III and IV cancer, 99.9% would be identified in the first step, of which 99.9%×99.9%=99.8%=53,568 individuals with late cancer would be identified. This brings the total identified to 38,896+40,176+53,892=132,964 individuals out of 135,000 with colorectal cancer, for an overall sensitivity of 98.5%. Overall, the positive predictive value of such a test would be 132,964/(188,748+132,964)=41.3%. In other words, 1 in 2.5 individuals who tested positive would actually have colorectal cancer, and this test would identify 79,072/81,000 or 97.6% of those individuals with early cancer, compared with the current rate of 40%.

How would these results vary for using the first strategy (FIG. 1D) for detection of early ovarian cancer, with the anticipation of an average of 150 methylated molecules per positive marker in the blood? Again, assume that the stages are evenly divided, and thus, individuals with Stage I ovarian cancer would be about 5,500 individuals. Assuming individual marker false-positive rates of 3%, the first step using 96 markers (36 markers for ovarian) with average sensitivities of 66%, and requiring a minimum of 5 markers positive, then, with an overall specificity of 99.1%, the first step would identify 486,000 individuals (out of 54,000,000 total women ages 50-79 in the U.S.). This would include, at 71.5% sensitivity, about 3,932 individuals with Stage I ovarian cancer (out of 5,500 individuals with Stage I ovarian cancer). However, those 486,000 presumptive positive individuals would be evaluated in a second step of 64 markers (36 markers for ovarian cancer) with average sensitivities of 66%, and requiring a minimum of 5 markers positive. The two-step test would identify 71.5%×71.5%=51.1%=2,810 individuals (out of 5,500 individuals with Stage I ovarian cancer) with ovarian cancer. With a specificity of 99.1%, the second test would also generate 486,000×0.9%=4,374 false-positives. The positive predictive value of such a test would be 2,810/(4,374+2,810)=39.1%. In other words, 1 in 2.5 individuals who tested positive would actually have Stage I ovarian cancer. In reality, one would need to also include the success for identifying Stage 2 and higher ovarian cancers. In expanding this example, the calculations are done with the anticipation that Stage I ovarian cancer has an average of 150 methylated molecules per positive marker in the blood, Stage II ovarian cancer has an average of 200 methylated molecules per positive marker, and the higher stages (III & IV) have at least an average of 300 methylated molecules per positive marker. Also, assuming all four stages are at 5,500, then 5,500×4=22,000 total individuals with ovarian cancer would be identified per year in the U.S. The above calculation already provided the false-positive rate for the early cancer. For Stage II cancer, 90.0% would be identified in the first step, of which 90.0%×90.0%=81.0%=4,485 individuals with Stage II ovarian cancer would be verified in the second step. For Stage III and IV ovarian cancer, 99.2% would be identified in the first step, of which 99.2%×99.2%=98.4%=10,824 individuals with late ovarian cancer would be identified. This brings the total identified at 2,810+4,485+10,824=18,119 individuals out of 22,000 with ovarian cancer, for an overall sensitivity of 82.4%. Overall, the positive predictive value of such a test would be 18,119/(18,119+4,374)=80.5%. In other words, 4 in 5 women who tested positive would actually have ovarian cancer, and this test would identify 7,295/11,000 or 66.3% of those individuals with early cancer, compared with the current rate of 15%.

How would these results vary for using this strategy (FIG. 1D) for detection of early ovarian cancer using 66% average marker sensitivities, with the anticipation that Stage I ovarian cancer has an average of 200 methylated molecules per positive marker in the blood, Stage II ovarian cancer has an average of 240 methylated molecules per positive marker, and the higher stages (III & IV) have at least an average of 300 methylated molecules per positive marker?

Assuming individual marker false-positive rates of 3%, the first step using 96 markers (36 markers for ovarian) with average sensitivities of 66%, and requiring a minimum of 5 markers positive, then, with an overall specificity of 99.1%, the first step would identify 486,000 individuals (out of 54,000,000 total women ages 50-79 in the U.S). This would include, at 90.0% sensitivity, about 4,950 individuals with Stage I ovarian cancer (out of 5,500 individuals with Stage I ovarian cancer). However, those 486,000 presumptive positive individuals would be evaluated in a second step of 64 markers (36 markers for ovarian cancer) with average sensitivities of 66%, requiring a minimum of 5 markers positive. The two-step test would identify 90.0%×90.0%=81.0%=4,895 individuals (out of 5,500 individuals with Stage I ovarian cancer) with ovarian cancer. With a specificity of 99.1%, the second test would also generate 486,000×0.9%=4,374 false-positives. The positive predictive value of such a test would be 4,895/(4,374+4,895)=52.8%. In other words, 1 in 2 individuals who tested positive would actually have Stage I ovarian cancer. In reality, one would need to also include the success for identifying Stage 2 and higher ovarian cancers. Assuming all four stages are at 5,500, then 5,500×4=22,000 total individuals with ovarian cancer would be identified per year in the U.S. The above calculation already provided the false-positive rate for the early cancer. For Stage II cancer, 96.2% would be identified in the first step, of which 96.2%×96.2%=92.5%=5,087 individuals with Stage II ovarian cancer would be verified in the second step. For Stage III and IV ovarian cancer, 99.2% would be identified in the first step, of which 99.2%×99.2%=98.4%=10,824 individuals with late ovarian cancer would be identified. This brings the total identified at 4,895+5087+10,824=20,806 individuals out of 22,000 with ovarian cancer, for an overall sensitivity of 94.6%. Overall, the positive predictive value of such a test would be 20,806/(20,806+4,374)=87.4%. In other words, 7 in 8 women who tested positive would actually have ovarian cancer, and this test would identify 9,982/11,000 or 90.1% of those individuals with early cancer, compared with the current rate of 15%.

The biology of each cancer is different, and thus the observed sensitivity and specificity for detecting early cancer, monitoring treatment, and detecting early recurrence may be higher or lower from the idealized calculations described herein.

### EXAMPLES

### Examples: Multiplex PCR-LDR-qPCR Detection of Cancer-Related Methylation Markers

### General Methods for Examples 1-5

HT-29 colon adenocarcinoma cells were seeded in 60 cm² culture dishes in McCoy's 5A medium containing 4.5 g/l glucose, supplemented with 10% fetal calf serum, and kept in a humidified atmosphere containing 5% CO₂. Once cells reached 80-90% confluence, they were washed in Phosphate Buffered Saline (×3), and collected by centrifugation (500×g). Genomic DNA was isolated using the DNeasy Blood & Tissue Kit (Qiagen; Valencia, Calif.), and its concentration measured using Quant-iT Pico green Assay (Life Technologies/Thermo-Fisher; Waltham, Mass.).

High molecular weight (>50 kb) genomic DNA (0.2 mg/ml) isolated from human blood (buffy coat) (Roche human genomic DNA) was purchased from Roche (Indianapolis, Ind.) Its concentration was similarly determined using Quant-iT PicoGreen dsDNA Assay Kit.

Cell free DNA was isolated from 5 ml plasma sample (with K₂EDTA additive as anti-coagulant) using the QIA amp Circulating Nucleic Acid Kit according to manufacturer's instructions, and quantified using Quant-iT Pico Green Assay (Life Technologies/ThermoFisher; Waltham, Mass.).

0.5-1.0 μg HT29 cell line genomic DNA was digested with 10 units of the restriction enzyme Bsh1236I in 20 μl of reaction solution containing 1×CutSmart buffer (50 mM Potassium Acetate, 20 mM Tris-Acetate, 10 mM Magnesium Acetate, 100 μg/ml BSA, pH 7.9 at 25° C.). The digestion reaction was carried out at 37° C. for 1 hour, followed by enzyme inactivation at 80° C. for 20 min. Alternatively, genomic DNAs can be fragmented through non random sonication method, using Covaris ultra sonicator (Woburn, Mass.). After shearing, the quality of the resulting DNA fragments (length ranged from 50 to 1 kb base pairs) was assessed with Agilent Bioanalyzer system. This is followed by an enrichment step wherein the DNA fragments containing methylated CpGs are then captured by methylation-specific antibodies, using the EpiMark® Methylated DNA Enrichment Kit according to manufacturer's instructions (New England Biolabs; Ipswich, Mass.).

PCR Primers, LDR Probes, and LNA or PNA Blocking Primers.

All primers used for the one or two-step assay to detect colorectal cancer are listed in Table 45 below. All primers were purchased from Integrated DNA Technologies Inc. (IDT) (Coralville, Iowa), except for LNA1 and LNA2, which were purchased from Exiqon Inc. (Woburn, Mass.), and PNA, which was purchased from PNA Bio (Thousand Oaks, Calif.). Primers designed for use in Step 1 of the 96-marker assay, with average sensitivities of 50%, detect solid tumors are listed in Table 46 below. Primers designed for use in Step 2 of the Group 1-64-marker assay, with average sensitivities of 50%, to detect and identify colorectal, stomach, and esophageal cancers are listed in Table 53 below. Primers designed for use in Step 2 of the Group 2-48-64-marker assay, with average sensitivities of 50%, to detect and identify breast, endometrial, ovarian, cervical, and uterine cancers are listed in Table 54 below. Primers designed for use in Step 2 of the Group 3-48-64-marker assay, with average sensitivities of 50%, to detect and identify lung adenocarcinomas, lung squamous cell carcinoma, and head & neck cancers are listed in Table 55 below. Primers designed for use in Step 2 of the Group 4-36-48-marker assay, with average sensitivities of 50%, to detect and identify prostate and bladder cancers are listed in Table 56 below. Primers designed for use in Step 2 of the Group 5-48-64-marker assay, with average sensitivities of 50%, to detect and identify liver, pancreatic, and gall-bladder cancers are listed in Table 57 below.

Lengthy table referenced here
US20220243263A1-20220804-T00001
Please refer to the end of the specification for access instructions.

Lengthy table referenced here
US20220243263A1-20220804-T00002
Please refer to the end of the specification for access instructions.

Lengthy table referenced here
US20220243263A1-20220804-T00003
Please refer to the end of the specification for access instructions.

Lengthy table referenced here
US20220243263A1-20220804-T00004
Please refer to the end of the specification for access instructions.

Lengthy table referenced here
US20220243263A1-20220804-T00005
Please refer to the end of the specification for access instructions.

Lengthy table referenced here
US20220243263A1-20220804-T00006
Please refer to the end of the specification for access instructions.

Lengthy table referenced here
US20220243263A1-20220804-T00007
Please refer to the end of the specification for access instructions.

Lengthy table referenced here
US20220243263A1-20220804-T00008
Please refer to the end of the specification for access instructions.

Lengthy table referenced here
US20220243263A1-20220804-T00009
Please refer to the end of the specification for access instructions.

Lengthy table referenced here
US20220243263A1-20220804-T00010
Please refer to the end of the specification for access instructions.

Lengthy table referenced here
US20220243263A1-20220804-T00011
Please refer to the end of the specification for access instructions.

Lengthy table referenced here
US20220243263A1-20220804-T00012
Please refer to the end of the specification for access instructions.

Lengthy table referenced here
US20220243263A1-20220804-T00013
Please refer to the end of the specification for access instructions.

### Example 1: Detection of VIM Promoter Methylation in HT29 Colorectal Cancer Cell Line Using exPCR-LDR-qPCR at the Single Molecule Level

500 ng HT29 cell line genomic DNA was digested with 10 units of restriction enzyme Bsh1236I in 20 μl of reaction solution containing 1×CutSmart buffer (50 mM Potassium Acetate, 20 mM Tris-Acetate, 10 mM Magnesium Acetate, 100 μg/ml BSA, pH 7.9 at 25° C.). The digestion reactions were carried out at 37° C. for 1 hour, followed by enzyme inactivation at 80° C. for 20 min.

The digests were then bisulfite converted using EZ DNA Methylation-Lightning kit from Zymo Research Corporation (Irvine, Calif.). In this reaction, 130 μl of Lightning Conversion Reagent was added to 20 μl of digested genomic DNA. The reaction was incubated at 98° C. for 8 minutes, 54° C. for one hour, and stopped at 4° C.

600 μl of M-Binding Buffer was added to a Zymo-Spin™ Column and the column inserted into a collection tube. The digested DNA (150 μl) from previous step was then loaded into the column. Capped, the column was inverted several times to mix the solution, then centrifuged at full speed (>10,000×g) for 30 sec. After discarding the flow through, 100 μl of M-washing buffer was added to the column, followed by another round of centrifugation at full speed for 30 second. The resulting flow through was then discarded. 200 μl of L-Desulphonation Buffer was then added to the column, and the column allowed to stand at room temperature for 15-20 minutes. After the incubation, the column was centrifuged at full speed for 30 seconds. 200 μl of M-Wash Buffer was then added to the column, followed by centrifugation at full speed for 30 second. The flow through was discarded. This wash step was repeated once. The column was then inserted into a 1.5 ml micro centrifuge tube, and 10 μl of M-Elution buffer was added to the column matrix. Finally, the column was centrifuged at full speed for 30 second to elute the DNA.

All the necessary primers (listed in Table 42) were purchased from Integrated DNA Technologies Inc. (IDT) (Coralville, Iowa), except for LNA1 and LNA2, which was purchased from Exiqon Inc. (Woburn, Mass.), and PNA, which was purchased from PNA Bio (Thousand Oaks, Calif.).

A 130 μl-volume PCR reaction was set up as follows: 23.14 μl of nuclease free water (IDT), 26 μl of Gotaq Flexi buffer 5× without Magnesium (Pro-mega, Madison, Wis.), 10.4 μl of MgCl₂ at 25 mM (Pro-mega, Madison, Wis.), 2.6 μl of dNTPs (10 mM each of dATP, dCTP, dGTP and dUTP) (Promega, Madison, Wis.), 3.25 μl of iCDx-2031-VIM-S3-FP forward primer at 2 μM, 3.25 μl of iCDx-2032-VIM-S3-PR reverse primer at 2 μM, 16.25 μl of iCDx-VIM-S3-LNA2 blocking primer at 2 μM, 3.25 μl of RNAseH2 (IDT) at 20 mU/μl, 2.86 μl of Klentaql polymerase (DNA Polymerase Technology, St. Louis, Mo.) mixed with Plantinim Taq Antibody (Invitrogen, Carlsbad, Calif.) (the mixture is prepared by adding 0.3 μl of Klentaql polymerase at 50 U/μl to 3 μl of Platinum Taq Antibody at 5 U/μl), and 39 μl of corresponding template. 39 μl of templates contains: (1) 0.070 ng (20 copy of Genome Equivalent GE) HT-29 DNA mixed with 9 ng (2500 GE) Roche hgDNA. (2) 0.035 ng (10 GE) HT-29 cell line DNA mixed with 9 ng (2500 GE) Roche hgDNA. (3) 9 ng (2500 GE) Roche DNA, (4) nuclease free water for the Non Template Control (NTC).

Each 130 μl PCR mixture was divided into 12 tubes, 10 μl each, and then the PCR reactions were run in a Proflex PCR system thermo-cycler (Applied Biosystems/ThermoFisher; Waltham, Mass.) with the following program: 2 min at 95° C., 35 cycles of (10 sec at 94° C., 30 sec at 60° C., and 30 sec at 72° C.), 10 min at 99.5° C., and a final hold at 4° C.

The LDR step was performed in a 10 μl reaction prepared by adding: 5.82 μl of nuclease free water (IDT), 1 μl of 10×AK16D ligase reaction buffer [1× buffer contains 20 mM Tris-HCl at pH 8.5 (Bio-Rad, Hercules, Calif.), 5 mM MgCl₂ (Sigma-Aldrich, St. Louis, Mo.), 50 mM KCl (Sigma-Aldrich), 10 mM DTT (Sigma-Aldrich) and 20 μg/ml of BSA (Sigma-Aldrich), 0.25 μl of DTT (Sigma-Aldrich) at 40 mM, 0.25 μl of NAD⁺ (Sigma-Aldrich) at 40 mM, 0.25 of RNAseH2 (IDT) at 20 mU/μl, 0.2 μl of iCDx-2033-Vim-S3-Up probe at 500 nM, 0.2 μl of iCDx-2034A-Vim-S3-Dn probe at 500 nM, 0.028 μl of purified AK16D ligase at 8.8 μM, and 2 μl of PCR reaction. LDR reactions were run in a Proflex PCR system thermocycler (Applied Biosystems) with the following program: 20 cycles of (10 sec at 94° C., and 4 min at 60° C.) followed by a final infinite hold at 4° C.

The qPCR step was performed in a 10 μl of reaction mixture prepared by adding: 1.5 μl of nuclease free water (IDT), 5 μl of 2×TaqMan® Fast Universal PCR Master Mix (consists of Amplitaq, UDG and dUTP) from Applied Biosystems (Life Technologies, Grand Island, N.Y.), 1 μl of iCDx-2036-Vim-S3-RT-FP forward primer at 2.5 μM, 1 μl of iCDx-2037-Vim-S3-RT-RP reverse primer at 2.5 μM, 0.5 μl of iCDx-2035-Vim-S3-RT-Pb Taqman™ probe at 5 μM, and 1 μl of LDR reaction products. The qPCR reactions were run in a ViiA7 real-time thermo-cycler from Applied Biosystems (Life Technologies, Grand Island, N.Y.), using MicroAmp® Fast-96-Well Reaction 0.1 ml plates sealed with MicroAmp™ Optical adhesive film (Applied Biosystems), with the following settings: fast block, Standard curve as experiment type, ROX as passive reference, Ct as quantification method (automatic threshold, but adjusted to 0.05 when needed), TAMRA as reporter, and NFQ-MGB as quencher. The program was set at: 2 min at 50° C., and 40 cycles of (1 sec at 95° C., and 20 sec at 60° C.).

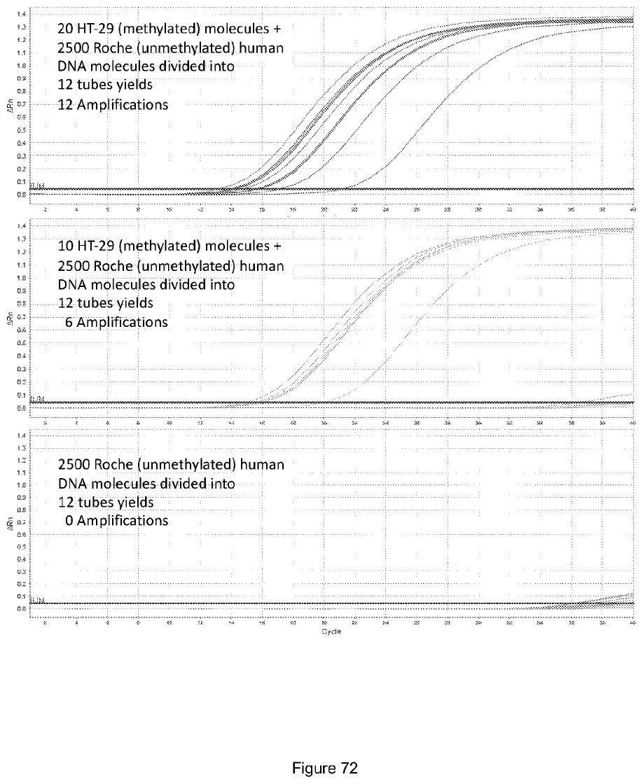

The pixel experiment results are shown in FIG. 72, while the resulting Ct values for different conditions are listed in Table 43. The following primer sequences from Table 58 were used in this Example: SEQ ID NOs.:1-10.

TABLE 58
Results of Pixel Experiments to Detect Methylation in HT-29 DNA in the Background
of Roche Human Genomic DNA. Ct Values in the Different Conditions.

| | GE per | | | | | | | | | | | | | |
|---|---|---|---|---|---|---|---|---|---|---|---|---|---|---|
| | 130 μl | | | | | | | | | | | | | Total No. |
| Templates | of PCR | 1 | 2 | 3 | 4 | 5 | 6 | 7 | 8 | 9 | 10 | 11 | 12 | Amplif. |
| HT29 + | 20 GE + | 21.5 | 17.5 | 14.5 | 16.0 | 15.9 | 14.4 | 15.0 | 14.5 | 14.3 | 14.2 | 16.1 | 13.5 | 12 |
| Roche | 2500 GE | | | | | | | | | | | | | |
| hgDNA | | | | | | | | | | | | | | |
| HT29 + | 10 GE + | No | 15.2 | No | 16.2 | No | 15.8 | 16.3 | No | 20.3 | 16.1 | No | No | 6 |
| Roche | 2500 GE | Ct | | Ct | | Ct | | | Ct | | | Ct | Ct | |
| hgDNA | | | | | | | | | | | | | | |
| Roche | 2500 GE | No | No | No | No | No | 38.5 | No | 36.8 | No | No | 37.7 | 36.6 | 0 |
| hgDNA | | Ct | Ct | Ct | Ct | Ct | | Ct | | Ct | Ct | | | |
| NTC | No Ct | No | 37.6 | No | 37.9 | 38.3 | No | No | No | 36.8 | 38.0 | 39.5 | No | 0 |
| | | Ct | | Ct | | | Ct | Ct | Ct | | | | Ct | |

### Example 2: Multiplexed Detection of 10 CRC Methylation Markers by PCR-LDR-qPCR on Bisulfite Converted HT29 Cell Line DNA

The experiment started with 500 ng of HT29 cell line genomic DNA digested with 12 units of the restriction enzyme Bsh1236I, in 20 μl of reaction solution containing 1×CutSmart buffer (50 mM Potassium Acetate, 20 mM Tris-Acetate, 10 mM Magnesium Acetate, 100 μg/ml BSA, pH 7.9 at 25° C.). The digestion reactions were carried out at 37° C. for 1 hour, followed by enzyme inactivation at 80° C. for 20 min.

For the bisulfate conversion reaction, 130 μl of Lightning Conversion Reagent (EZ DNA Methylation-Lightning kit from Zymo Research Corporation; Irvine, Calif.) was first added to 20 μl of digested genomic DNA. This is followed by incubation at 98° C. for 8 minutes, 54° C. for one hour, and stopped at 4° C. 600 μl of M-Binding Buffer was then added to a Zymo-Spin™ column placed inside a collection tube. 150 μl of digested DNA reaction mixture was then loaded into the Zymo-Spin IC column (containing the M-Binding Buffer), capped, and the solution mixed by inverting the column several times. After centrifugation at full speed (>10,000×g) for 30 sec, the flow through was discarded. 100 μl of M-Wash buffer was added to the column, followed by another round of centrifugation (full speed for 30 seconds). After discarding the flow through, 200 μl of L-Desulfonation Buffer was added into the column, which was allowed to stand at room temperature for 15-20 minutes, prior to centrifugation at full speed for 30 seconds. 200 μl of M-Wash Buffer was added anew, followed by centrifugation at full speed for 30 seconds, and discarding the flow through. This wash step cycle was repeated once. The column was placed into a 1.5 ml micro centrifuge tube, to which 10 μl of M-Elution buffer was added, followed by full speed (30 s) centrifugation to elute the DNA.

Most of the primers used in this particular experiment were purchased from Integrated DNA Technologies Inc. (IDT; Coralville, Iowa). The primers LNA1 and LNA2 were purchased from Exiqon Inc. (Woburn, Mass.), while the primer PNA was purchased from PNA Bio (Thousand Oaks, Calif.). The following primer sequences from Table 45 were used in PCR-LDR-qPCR multiplex methylation assays for 10 genes using HT29 cell line DNA as template: SEQ ID NOs.:1, 2, and 6-75.

PCR reaction. The PCR reaction was performed by mixing the following (in a 10 μl volume): 2 μl of Gotaq Flexi buffer 5× without Magnesium (Promega, Madison, Wis.), 0.8 μl of MgCl₂ at 25 mM (Promega, Madison, Wis.), 0.2 μl of dNTPs (with dATP, dCTP, dGTP and dUTP; 10 mM each) (Promega, Madison, Wis.), 0.125 μl of 10 specific forward and reverse primers at 4 μM each. 0.625 μl of Blocker at 4 μM (if available), 0.25 μl of RNAseH2 (IDT) at 20 mU/μl (diluted in RNAseH2 dilution buffer from IDT original RNAseH2 at 2 U/μl), 0.22 μl of Klentaql polymerase (DNA Polymerase Technology, St. Louis, Mo.) mixed with Platinum Taq Antibody (Invitrogen/Thermo Fisher, Waltham, Mass.) (the mixture is prepared by adding 0.02 μl of Klentaql polymerase at 50 U/μl to 0.2 μl of Platinum Taq Antibody at 5 U/μl), DNA template: (1) 10 ng of bisulfite converted HT29 DNA, (2) 0.07 ng of bisulfite converted HT29 DNA and 10 ng bisulfite converted normal DNA, (3) 10 ng bisulfite converted normal DNA. PCR reactions were carried out in a ProFlex PCR system thermocycler (Applied Bio-systems/ThermoFisher, Waltham, Mass.) and run with the following program: 94° C. for 2 min, 40 cycles of (20 sec at 94° C., 40 sec at 60° C. and 30 sec at 72° C.), 10 min at 99.5° C. to inactivate Klan Taq Polymerase, and a final hold at 4° C.

LDR Step.

The LDR step was performed in a 10 μl reaction prepared by adding: 2.02 μl of nuclease free water (IDT), 1 μl of 10×AK16D ligase reaction buffer [1× buffer contains 20 mM Tris-HCl, pH 8.5 (Bio-Rad, Hercules, Calif.), 5 mM MgCl₂ (Sigma-Aldrich, St. Louis, Mo.), 50 mM KCl (Sigma-Aldrich, St. Louis, Mo.), 10 mM DTT (Sigma-Aldrich, St. Louis, Mo.) and 20 μg/ml of BSA (Sigma-Aldrich, St. Louis, Mo.)], 0.25 μl of DTT (Sigma-Aldrich, St. Louis, Mo.) at 40 mM, 0.2 μl of NAD+ (Sigma-Aldrich, St. Louis, Mo.) at 50 mM, 0.25 μl of RNAseH2 (IDT) at 20 mU/μ1, 0.2 μl of corresponding 10 gene LDR upstream probes at 500 nM, 0.2 μl of corresponding 10 gene LDR downstream probes at 500 nM, 0.284 μl of purified AK16D ligase at 0.88 μM, and 2 μl of PCR reaction products from previous step. LDR reactions were run in a ProFlex PCR system thermocycler (Applied Biosystems/ThermoFisher; Waltham, Mass.) using the following program: 20 cycles of (10 sec at 94° C., and 4 min at 60° C.) followed by a hold at 4° C.

qPCR Step.

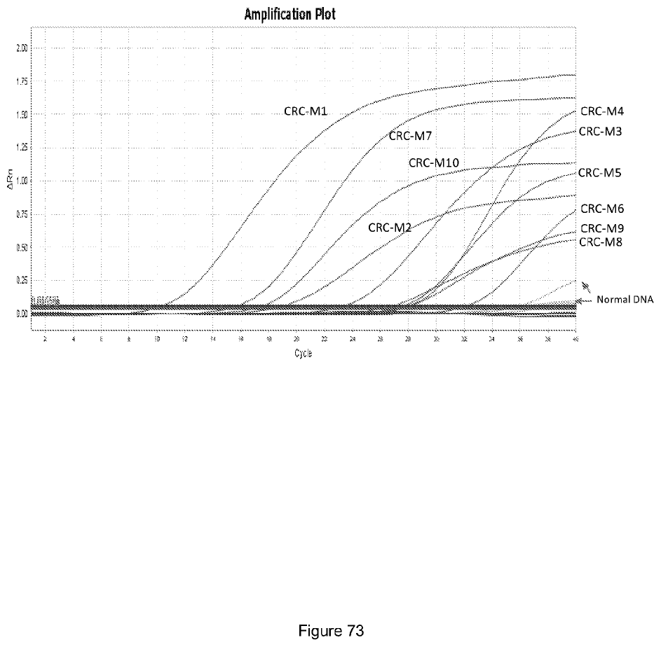

The qPCR reactions were performed as uniplex, wherein each reaction mixture contains only one set of gene-specific primers and probe. A 10 μl reaction mixture was prepared by mixing: 1.5 μl of nuclease free water (IDT), 5 μl of 2×TaqMan® Fast Universal PCR Master Mix (Fast amplitaq, UDG and dUTP) from Applied Biosystems (Applied Biosystems/ThermoFisher; Waltham, Mass.), 1 μl of TaqMan™ Assay forward primer at 2.5 μM, 1 μl of Taqman™ reverse primer at 2.5 μM, 0.5 μl of Taqman™ probe at 5 μM, and 1 μl of LDR reaction products. qPCR reactions were run in a ViiA7 real-time thermo-cycler from Applied Biosystems (Applied Biosystems/Thermo-Fisher; Waltham, Mass.), using MicroAmp® Fast-96-Well Reaction 0.1 ml plates sealed with MicroAmp™ Optical adhesive film (Applied Biosystems/ThermoFisher; Waltham, Mass.), with the following setting: fast block, Standard curve as experiment type, ROX as passive reference, Ct as quantification method (automatic threshold, but adjusted to 0.05 when needed), TAMRA as reporter, and NFQ-MGB as quencher. The specific program used was the following: 2 min at 50° C., and 45 cycles of (1 sec at 95° C., and 20 sec at 60° C.). Results are shown in FIG. 73 and Ct Values are shown in Table 59.

TABLE 59
Ct Values for Genes Assayed in Example 2.

| | CLIP4- | GSG1L- | | | | LONF | TBC1D10C- | GDF6- | CLIP4- | SEPT9- |
|---|---|---|---|---|---|---|---|---|---|---|
| Ct Value | R-S1 | F-S1 | PP1R16B | KCNA3 | VIM-S3 | R2-R-S1 | F-S1 | F-S1 | F-S2 | F-S1 |
| 10 ng | 7.2 | 9.1 | 15.8 | 19.6 | 19.7 | 25.1 | 14.2 | 17.3 | 20.9 | 11.2 |
| HT29 | | | | | | | | | | |
| DNA | | | | | | | | | | |
| 0.07 ng | 10.2 | 18.8 | 23.2 | 27.8 | 27.4 | 31.9 | 15.6 | 26.6 | 27.9 | 17.4 |
| HT29 | | | | | | | | | | |
| DNA + | | | | | | | | | | |
| 10 ng | | | | | | | | | | |
| Roche | | | | | | | | | | |
| 10 ng | No Ct | No Ct | 35.6 | 35.8 | No Ct | 38.4 | 36.7 | No Ct | No Ct | No Ct |
| Roche | | | | | | | | | | |
| DNA | | | | | | | | | | |
| NTC_PCR | No Ct | No Ct | 36.1 | 36.7 | No Ct | No Ct | No Ct | No Ct | No Ct | No Ct |
| NTC_LDR | No Ct | 38.2 | 36.0 | No Ct | No Ct | No Ct | No Ct | No Ct | No Ct | 37.7 |
| NTC_Taq | No Ct | No Ct | No Ct | No Ct | No Ct | No Ct | No Ct | No Ct | No Ct | No Ct |
| Man ™ | | | | | | | | | | |

Note:
NTC: No Template Control

### Example 3: Multiplexed Detection of 7 CRC Methylation Markers Using ex-PCR-LDR-qPCR on Bisulfite Converted HT29 Cell Line DNA

In a 20 μl reaction volume, 500 ng HT29 cell line genomic DNA was mixed with 12 units of restriction enzyme Bsh1236I, in 1×CutSmart buffer (50 mM Potassium Acetate, 20 mM Tris-Acetate, 10 mM Magnesium Acetate, 100 μg/ml BSA, pH7.9@25° C.). This digestion reactions were carried out at 37° C. for 1 hour, followed by enzyme inactivation at 80° C. for 20 min. Bisulfite conversion was carried out using the Cells-to-CpG Bisulfite Conversion kit from Applied Biosystem Corporation (Carlsbad, Calif.). For this reaction, 5 μl of Denaturation Reagent was added to 45 μl of restriction digested or methyl enriched genomic DNA, and the mixture was incubated at 50° C. for 10 min (to denature the DNA). 100 μl of Conversion Reagent was added to the mixture, followed by incubation in a thermal cycler with the following cycling conditions: 65° C. 30 min, 90° C. 30 sec, 65° C. 30 min, 90° C. 30 sec, 65° C. 30 min. 150 μl of the bisulfite converted DNA mixture was then mixed with 600 μl of binding buffer in the binding Column. The column was centrifuged at 10,000 rpm for 1 min, and the flow through discarded thereafter. The column was then washed with 600 μl of washing buffer. This was followed with addition of 200 μl of Desulfonation Reagent, and the column incubation at room temperature for 15 min. After spinning, the column was washed again with 400 μl of washing buffer. 50 μl of Elution Buffer was then added to the column to elute the DNA. Since the eluted bisulfite converted DNA was mostly single stranded DNA, it was quantified using Quant-iT Oli Green and Pico Green kits (Life Technologies/ThermoFisher; Waltham, Mass.).

The necessary primers were mostly purchased from Integrated DNA Technologies Inc. (IDT; Coralville, Iowa). The LNA1 and LNA2 primers were purchased from Exiqon Inc. (Woburn, Mass.), while the PNA primer, which was purchased from PNA Bio (Thousand Oaks, Calif.). The following primer sequences from Table 45 were used in Example 3: SEQ ID NOs.:1, 2, 6-39, 54-60, and 68-75.

Linear Amplification Step.

The Linear Amplification step was performed in a 25 μl of reaction mixture with: 5 μl of Gotaq Flexi buffer 5× without Magnesium (Promega, Madison, Wis.), 2.5 μl of MgCl₂ at 25 mM (Promega, Madison, Wis.), 0.5 μl of dNTPs (with dATP, dCTP, dGTP and dTTP, 10 mM each) (Promega, Madison, Wis.), 1.25 μl of 7 plex gene specific one direction primers (concentration of primer for each gene is 2 μM), 0.625 μl of RNAseH2 (IDT) at 20 mU/μl (diluted in RNAseH2 dilution buffer from IDT), and 0.55 μl of Klentaql polymerase (DNA Polymerase Technology, St. Louis, Mo.) mixed with Platinum Taq Antibody (Invitrogen/Thermo Fisher, Waltham, Mass.) (the mixture is prepared by adding 0.02 μl of Klentaql polymerase at 50 U/μl to 0.2 μl of Platinum Taq Antibody at 5 U/μl), and 14.5 μl of corresponding bisulfite converted genomic DNA (out of 50 μl of eluted DNA after bisulfite conversion.) DNA templates were: (1) 10 ng of bisulfite converted HT29 DNA, (2) 0.1 ng bisulfite converted HT29 mixed with 10 ng bisulfite converted Normal DNA from Roche, (3) 10 ng bisulfite converted Normal DNA from Roche. Linear Amplification reactions were run in a ProFlex PCR system thermocycler (Applied Biosystems/ThermoFisher, Waltham, Mass.) and run with the following program: 2 min at 94° C., 40 cycles of (20 sec at 94° C., 40 sec at 60° C. and 30 sec at 72° C.), a final hold at 4° C. After the reaction, Platinum Taq antibodies were added in the reaction mixture to inhibit the Klentaq DNA polymerase.

PCR Reaction.

The linear amplification products were equally divided into two parts (4-plex for 4 CpG markers, and 3-plex for 3 CpG markers). The PCR step was performed in a 20 μl reaction volume by prepared by mixing: 2 μl of 5× GoTaq Flexi buffer without Magnesium (Promega, Madison, Wis.), 1 μl of MgCl₂ at 25 mM (Promega, Madison, Wis.), 0.4 μl of dNTPs (with 10 mM each for dATP, dCTP, dGTP and dUTP) (Promega, Madison, Wis.), 1 μl of each of the opposite-strand PCR primer (2 μM concentration), 0.4 μl of Antarctic Thermolabile UDG (1 u/μl)(New England Biolab, Ipswich, Mass.), 0.25 μl of RNAseH2 (IDT) at 20 mU/μl, 0.44 μl of Klentaql polymerase (DNA Polymerase Technology, St. Louis, Mo.) mixed with Platinum Taq Antibody (Invitrogen/Thermo Fisher, Waltham, Mass.) (the mixture is prepared by adding 0.02 μl of Klentaql polymerase at 50 U/μl to 0.2 μl of Platinum Taq Antibody at 5 U/μl), and 12 μl of corresponding linear amplification products. PCR reactions were run in a ProFlex PCR system thermocycler (Applied Biosystems/ThermoFisher, Waltham, Mass.) with the following program: 10 min at 37° C., 40 cycles of (20 sec at 94° C., 40 sec at 60° C. and 30 sec at 72° C.), 10 min at 99.5° C., and a final hold at 4° C.

LDR Step.

The LDR step was performed in a 20 μl of reaction volume by mixing: 11.6 μl of nuclease free water (IDT), 2 μl of 10× AK16D ligase reaction buffer [1× buffer contains 20 mM Tris-HCl pH 8.5 (Bio-Rad, Hercules, Calif.), 5 mM MgCl₂ (Sigma-Aldrich, St. Louis, Mo.), 50 mM KCl (Sigma-Aldrich, St. Louis, Mo.), 10 mM DTT (Sigma-Aldrich, St. Louis, Mo.) and 20 μg/ml of BSA (Sigma-Aldrich, St. Louis, Mo.), 0.5 μl of DTT (Sigma-Aldrich, St. Louis, Mo.) at 40 mM, 0.4 μl of NAD+(Sigma-Aldrich, St. Louis, Mo.) at 50 mM, 0.5 μl of RNAseH2 (IDT) at 20 mu/μ1, 0.4 μl of corresponding 4 plex LDR upstream probes at 500 nM, 0.4 μl of corresponding 4 plex LDR downstream probes at 500 nM, (3 plex for the other LDR reaction), 0.57 μl of purified AK16D ligase at 0.88 μM, and 4 μl of corresponding PCR reaction products from previous step. LDR reactions were run in a ProFlex PCR system thermocycler (Applied Biosystems/ThermoFisher; Waltham, Mass.) using the following program: 20 cycles of (10 sec at 94° C., and 4 min at 60° C.) followed by a final hold at 4° C.

qPCR Step.

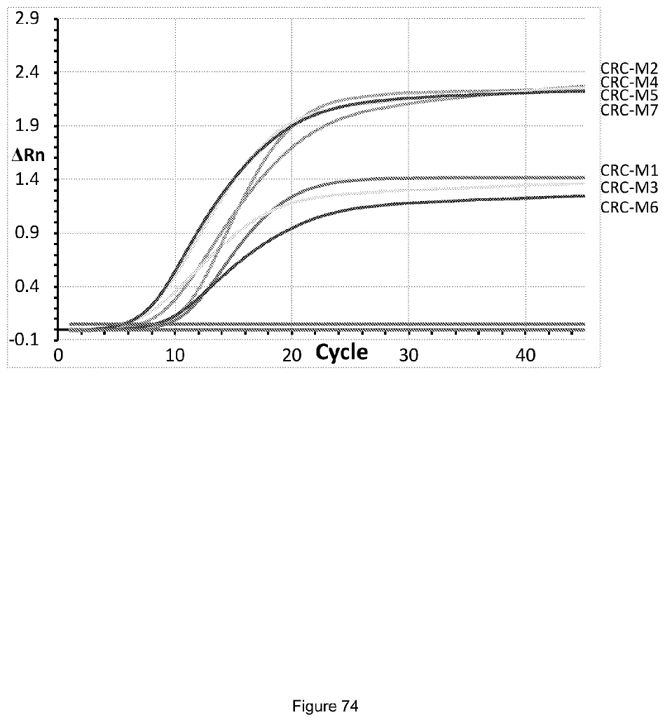

Uni-plex qPCR reactions (i.e. each reaction tube contains only the primers and probe specific to a unique CpG site) were performed in a 10 μl reaction mixture containing: 3 μl of nuclease free water (IDT), 5 μl of 2×TaqMan™ Fast Universal PCR Master Mix (Fast Amplitaq, UDG and dUTP) from Applied Biosystems (Applied Biosystems/ThermoFisher; Waltham, Mass.), 0.5 μl of mixture of TaqMan™ Assay forward and reverse primers at 5 μM each, 0.5 μl of Taqman™ probe at 5 μM, and 1 μl of corresponding LDR reaction products. The reactions were run in a ViiA7 real-time thermo-cycler from Applied Biosystems (Applied Biosystems/ThermoFisher; Waltham, Mass.), using MicroAmp® Fast-96-Well Reaction 0.1 ml plates sealed with MicroAmp™ Optical adhesive film (Applied Biosystems/ThermoFisher; Waltham, Mass.), with the following setting: fast block, Standard curve as experiment type, ROX as passive reference, Ct as quantification method (automatic threshold, but adjusted to 0.05 when needed), TAMRA as reporter, and NFQ-MGB as quencher. The thermocycler was programmed as follows: 2 min at 50° C., and 45 cycles of (1 sec at 95° C., and 20 sec at 60° C.). The Ct plots and values are shown in FIG. 74 and Ct Table 60, respectively.

TABLE 60
Ct Values for 7 Genes in Example 3.

| | | 01 ng | | | | |
|---|---|---|---|---|---|---|
| | | HT29 + 10 | 10 ng | | | |
| | 10 ng | ng Roche | Roche | | | |
| CT Values | HT29 | Normal | Normal | NTC_PCR | NTC_LDR | NTC_TaqMan ™ |
| VIM-S3 | 5.9 | 9.1 | No Ct | No Ct | No Ct | No Ct |
| CLIP4-R-S1 | 6.7 | 7.0 | 31.8 | 31.9 | 31.5 | No Ct |
| GSG1L-F-S1 | 4.9 | 5.6 | No Ct | No Ct | No Ct | No Ct |
| PP1R16B-F-S1 | 4.7 | 8.7 | 34.3 | 35.7 | 36.0 | No Ct |
| KCNA3-F-S1 | 5.4 | 5.5 | 25.0 | 30.5 | 37.1 | No Ct |
| GDF6-F-S1 | 6.8 | 8.2 | 7.3 | NO Ct | No Ct | No Ct |
| SEPT9-F-S1 | 5.3 | 5.5 | No Ct | No Ct | No Ct | No Ct |

### Example 4: Multiplexed Detection of 7 CRC Methylation Markers Using Ex-PCR-LDR-qPCR on Bisulfite Converted Cell Free DNA in Tumor Plasma

Human plasma (with K2-EDTA as an anti-coagulant) samples were purchased from a vendor. Cell free DNA was isolated from individual plasma samples (5 mL) using the QIA amp Circulating Nucleic Acid Kit (Qiagen) according to manufacturer's instructions, and quantified with Quant-iT Pico Green Assay Life Technologies/Thermo-Fisher; Waltham, Mass.). CpG Methylated cell free DNA fragments were enriched by antibodies containing methyl-CpG binding domain. A series of wash steps, magnetic capture, and incubation at 65° C., were employed prior to elution of methylation-enriched cell free DNA.

Bisulfite conversion of enriched methylated cell free DNA was carried out using the innuCONVERT bisulfate Body Fluids kit from Analytic Jena Corporation (Jena, Germany). Conversion reaction was carried out in 150 μl of mixture by adding 50 μl of enriched cell free DNA, 70 μl of Conversion Reagent, 30 μl of Conversion Buffer. The mixture was incubated in a thermal mixer at 85° C. for 45 min, with shaking at 800 rpm speed. After incubation, 700 μl of binding buffer was added to the reaction mixture, prior to being loaded into a spin column, then centrifuged. 200 μl of washing solution BS was then added to the column, prior to centrifugation. This is followed by addition of 700 μl of desulfonation buffer to the column, and incubation at room temperature for 10 minutes. The column was then subjected to a series of wash steps: 500 μl of washing solution C, 650 μl of washing solution BS, 650 μl of ethanol (twice). The column was then incubated at 60° C. for 10 min to remove residue ethanol. The DNA was finally eluted with 50 μl of elution buffer.

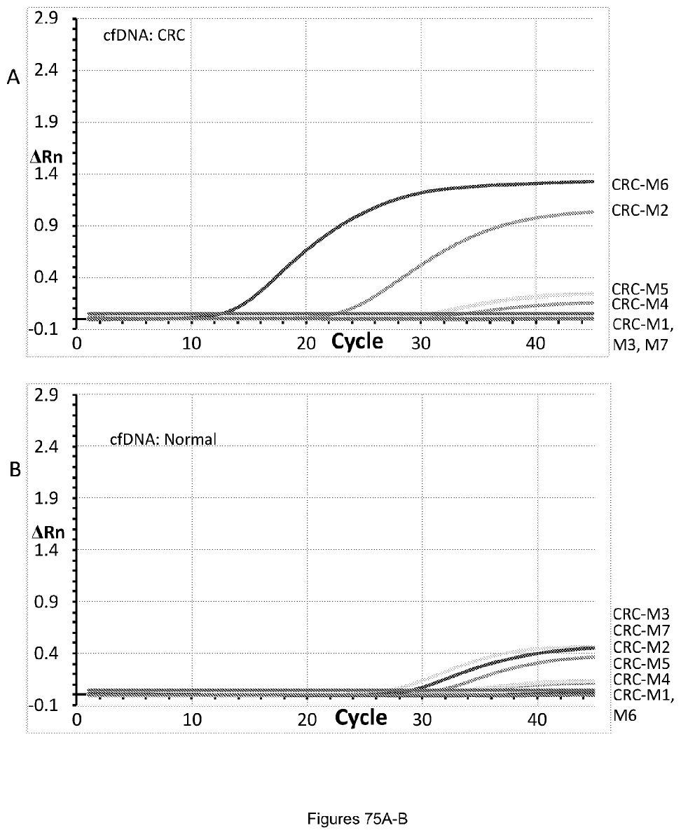

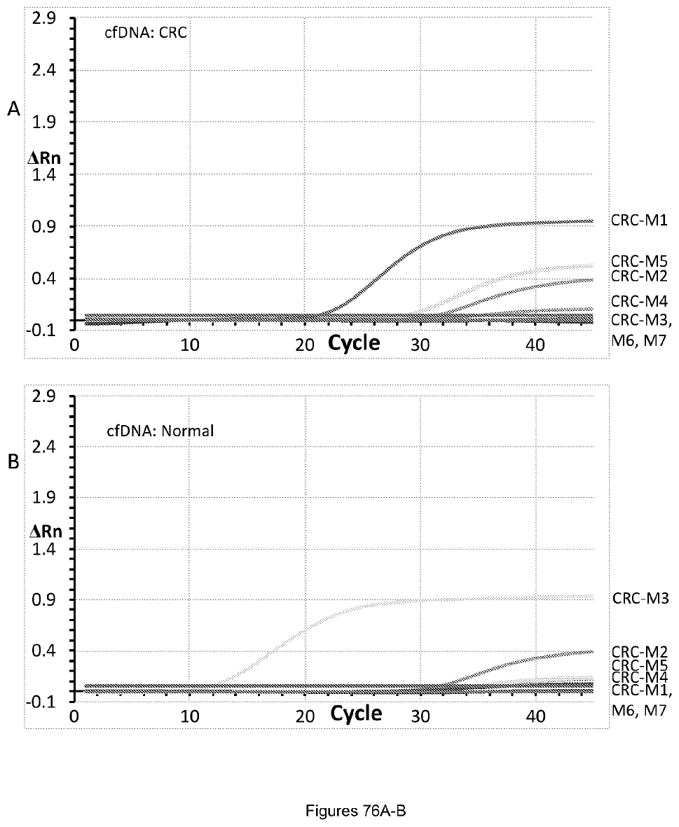

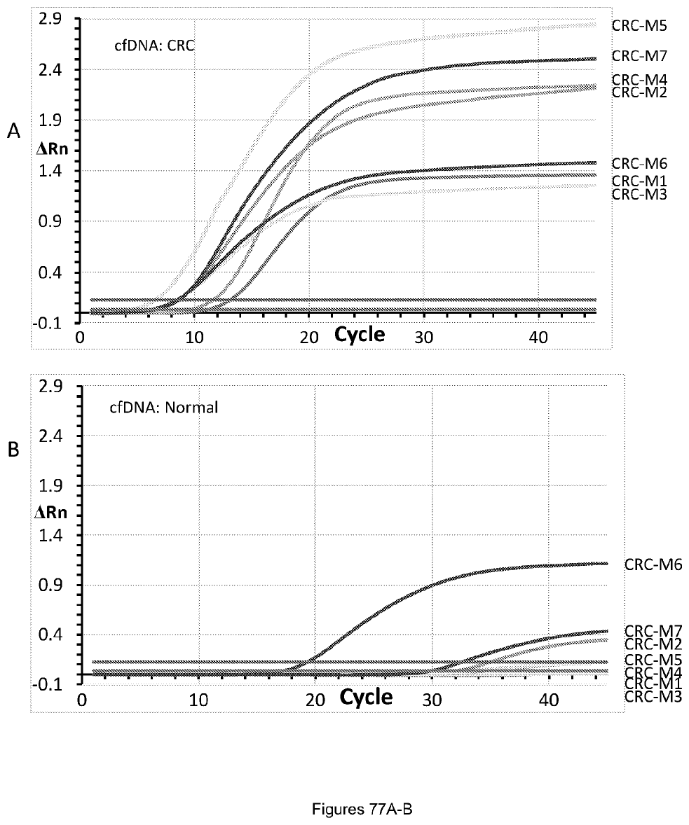

The primers used for the example described above were identical to those used in Example 3. The linear amplification, PCR, LDR and qPCR steps were carried out as described in Example 3. Three sets of cell free DNA samples (a set consists of CRC patient/normal pair) were tested using this protocol. The results using cfDNA isolated from the plasma of 3 different CRC patients and 3 different normal controls are shown in FIGS. 75, 76, and 77.

### Example 5: Multiplexed Detection of 20 CRCM Markers Using ex-PCR-LDR-qPCR on Bisulfite Converted HT29 Cell Line DNA

The template for this particular example was also the genomic DNA extracted from HT29, and fragmented though Non Random Sonication method using ultra-sonicator from Covaris (Woburn, Mass.). After shearing, DNA quality was assessed with an Agilent Bioanalyzer system. The length of DNA ranged from 50 bp to 1 kb. DNA in the elution buffer was quantified using Pico Green kit (Life Technologies/Thermo-Fisher; Waltham, Mass.).

DNA fragments containing methylated CpG sites were enriched by binding to the antibodies containing methyl-CpG binding domain. After a series of wash steps followed by magnetic capture, the enriched DNA sample is eluted in a small volume of water by incubation at 65° C.

Bisulfite conversion was then carried out using the Cells-to-CpG Bisulfite Conversion kit from Applied Biosystem division of ThermoFisher (Carlsbad, Calif.). 5 μl of Denaturation Reagent was added to 45 μl of methyl enriched genomic DNA, followed by the mixture's incubation at 50° C. for 10 min. This is followed by addition of 100 μl of Conversion Reagent, and incubation in a thermal cycler with the following program: 65° C. 30 min, 90° C. 30 sec, 65° C. 30 min, 90° C. 30 sec, 65° C. 30 min. 150 μl of converted DNA mixture was mixed with 600 μl of binding buffer in the binding Column. The column was centrifuged at 10,000 rpm for 1 min, followed by discarding the flow through. The column was washed with 600 μl of washing buffer. 200 μl of Desulfonation Reagent was added to the column, followed by incubation at room temperature for 15 min. After spinning, the column was washed again with 400 μl of washing buffer. 50 μl of Elution Buffer was then added to the column to elute the bound DNA. The mostly single stranded, bisulfite converted DNA was quantified with both Quant-iT Oli Green and Pico Green kit (Life Technologies/ThermoFisher; Waltham, Mass.).

All primers used in the preceding example were purchased from Integrated DNA Technologies Inc. (IDT; Coralville, Iowa). The following primer sequences from Table 45 were used in Example 5: SEQ ID NOs.: 76-235.

Linear Amplification Step.

In a 25 μl of reaction volume, the linear amplification step was performed by mixing: 5 μl of 5× GoTaq Flexi buffer (no Magnesium) (Promega, Madison, Wis.), 2.5 μl of 25 mM MgCl₂ (Promega, Madison, Wis.), 0.5 μl of 10 mM dNTPs (dATP, dCTP, dGTP and dTTP) (Promega, Madison, Wis.), 2.5 μl of 20 plex gene specific reverse primers (concentration of each primer is 1 μM), 0.625 μl of 20 mU/μl RNAseH2 (diluted in RNAseH2 dilution buffer from IDT) (DT), and 0.55 μl of Klentaql polymerase (DNA Polymerase Technology, St. Louis, Mo.) mixed with Platinum Taq Antibody (Invitrogen/Thermo Fisher, Waltham, Mass.) (the mixture is prepared by adding 0.02 μl of Klentaql polymerase at 50 U/μl to 0.2 μl of Platinum Taq Antibody at 5 U/μl), and 5.0 μl of corresponding bisulfite converted genomic DNA (out of 50 μl of eluted DNA after bisulfite conversion.) The template was either: 1) 1.0 μg of Normal Human genomic DNA (purchased from Roche) mixed with 66.0 ng of HT29 colorectal cell line genomic DNA, or 2) just 1.0 μg of Normal Human genomic DNA (normal control). The template was either digested with restriction enzyme Bsh1236I, or enriched in methylated DNA, bisulfite converted, and eluted into 50 μl of elution buffer. 5 μl of elution buffer was used in the linear amplification reaction. The reactions were run in a ProFlex PCR system thermocycler (Applied Biosystems/ThermoFisher, Waltham, Mass.) using the following program: 2 min at 94° C., 40 cycles of (20 sec at 94° C., 40 sec at 60° C., and 30 sec at 72° C.), and a final hold at 4° C. After the reaction, Platinum Taq Antibodies were added in the reaction mixture to inhibit the Klentaq DNA polymerase.

PCR Reaction.

The linear amplification products were equally divided into two parts. In the 1^{st} part, first 10 plex (out of 20 plex) gene specific forward primers and other reagents were added, in the 2^{nd} part, the other 10 plex (out of 20 plex) gene specific forward primers and other reagents were added. Two 10 plex PCR reactions were carried out. The PCR step was performed in a 20 μl of reaction mixture prepared by adding: 2 μl of GoTaq Flexi buffer 5× without Magnesium (Promega, Madison, Wis.), 1 μl of MgCl2 at 25 mM (Promega, Madison, Wis.), 0.4 μl of dNTPs (with dATP, dCTP, dGTP and dUTP, 10 mM each) (Promega, Madison, Wis.), 2 μl of 10 plex (out of 20 plex in linear amplification step) gene specific forward primers at 0.5 μM each. 0.4 μl of Antarctic Thermolabile UDG (1 u/μl) (New England Biolab, Ipswich, Mass.), 0.25 μl of RNAseH2 (IDT) at 20 mU/μl, 0.44 μl of Klentaql polymerase (DNA Polymerase Technology, St. Louis, Mo.) mixed with Platinum Taq Antibody (Invitrogen/Thermo Fisher, Waltham, Mass.) (The mixture is prepared by adding 0.02 μl of Klentaql polymerase at 50 U/μl to 0.2 μl of Platinum Taq Antibody at 5 U/μl), and 10 μl of corresponding linear amplification products. PCR reactions were run in a ProFlex PCR system thermocycler (Applied Biosystems/ThermoFisher, Waltham, Mass.) and using the following program: 10 min at 37° C., 40 cycles of (20 sec at 94° C., 40 sec at 60° C. and 30 sec at 72° C.), 10 min at 99.5° C., and a final hold at 4° C.

LDR Step.

The LDR step was performed in a 20 μl reaction prepared by combining: 5.82 μl of nuclease free water (IDT), 2 μl of 10× AK16D ligase reaction buffer [1× buffer contains 20 mM Tris-HCl pH 8.5 (Bio-Rad, Hercules, Calif.), 5 mM MgCl₂ (Sigma-Aldrich, St. Louis, Mo.), 50 mM KCl (Sigma-Aldrich, St. Louis, Mo.), 10 mM DTT (Sigma-Aldrich, St. Louis, Mo.) and 20 μg/ml of BSA (Sigma-Aldrich, St. Louis, Mo.), 0.5 μl of 40 mM DTT (Sigma-Aldrich, St. Louis, Mo.), 0.25 μl of 40 mM NAD+(Sigma-Aldrich, St. Louis, Mo.), 0.5 μl of 20 mU/μl RNAseH2 (IDT), 0.4 μl of corresponding 10 plex LDR upstream probes at 500 nM each, 0.4 μl of corresponding 10 plex LDR downstream probes at 500 nM each, 0.57 μl of purified AK16D ligase (at 0.88 μM), and 4 μl of PCR reaction products from previous step. LDR reactions were run in a ProFlex PCR system thermocycler (Applied Biosystems/Thermo-Fisher; Waltham, Mass.) using the following program: 20 cycles of (10 sec at 94° C., and 4 min at 60° C.) followed by a final hold at 4° C.

qPCR Step.

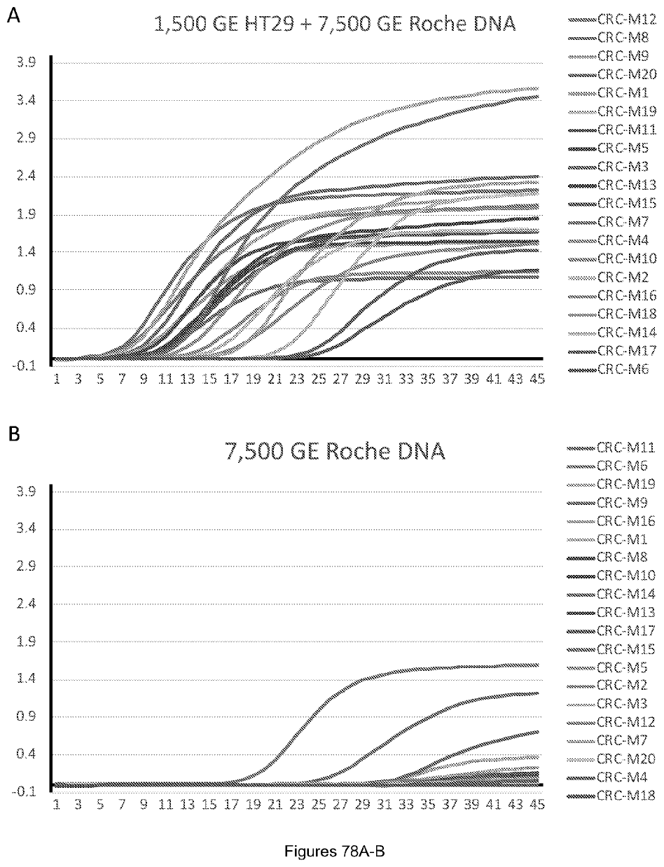

The qPCR step was performed in a 10 μl of reaction volume by combining: 1.5 μl of nuclease free water (IDT), 5 μl of 2×TaqMan™ Fast Universal PCR Master Mix (Fast amplitaq, UDG and dUTP) from Applied Biosystems (Applied Biosystems/ThermoFisher; Waltham, Mass.), 1 μl of 2.5 μM TaqMan™ Assay forward primer, 1 μl of 2.5 μM Taqman™ reverse primer, 0.5 μl of 5 μM Taqman™ probe, and 1 μl of LDR reaction products. qPCR reactions were run in a ViiA7 real-time thermo-cycler from Applied Biosystems (Applied Biosystems/Thermo-Fisher; Waltham, Mass.), using MicroAmp® Fast-96-Well Reaction 0.1 ml plates sealed with MicroAmp™ Optical adhesive film (Applied Biosystems/ThermoFisher; Waltham, Mass.), with the following setting: fast block, Standard curve as experiment type, ROX as passive reference, Ct as quantification method (automatic threshold, but adjusted to 0.05 when needed), TAMRA as reporter, and NFQ-MGB as quencher. The program employed was: 2 min at 50° C., and 45 cycles of (1 sec at 95° C., and 20 sec at 60° C.). Results are shown in FIG. 78, and Table 61 below.

TABLE 61
Ct values for each gene in Example 5.

| | Ct Values | | | | | | | | | |
|---|---|---|---|---|---|---|---|---|---|---|
| | 1 | 2 | 3 | 4 | 5 | 6 | 7 | 8 | 9 | 10 |
| | Gene | | | | | | | | | |
| | GATA5- | ZNF542- | RCN- | MYO15B- | ANKRD13B- | FAM115A- | RGS10- | HCG4- | STK32B- | CNRIP1- |
| | S2 | S1 | 3-S1 | S1 | S1 | S1 | S1 | S1 | S1 | S1 |
| starting | 5421 | 5431 | 5441 | 5451 | 5461 | 5471 | 5491 | 5501 | 5511 | 5521 |
| number | | | | | | | | | | |
| for primer | | | | | | | | | | |
| 1,000 GE of | 17.0 | 12.6 | 12.6 | 10.0 | 12.9 | 16.5 | 8.8 | 10.9 | 6.6 | 11.0 |
| HT29 DNA + | | | | | | | | | | |
| 7,500 GE of | | | | | | | | | | |
| Roche DNA | | | | | | | | | | |
| 7,500 GE of | 34.2 | 39.4 | 39.8 | No Ct | 37.5 | 24.7 | No Ct | 35.7 | 31.4 | 35.7 |
| Roche DNA | | | | | | | | | | |
| 1st Linaer | 34.4 | 39.6 | 39.6 | 36.3 | No Ct | No Ct | No Ct | 37.0 | 31.6 | 38.4 |
| Amp_NTC | | | | | | | | | | |
| 2nd Linear | 32.6 | 36.6 | 37.9 | 38.3 | No Ct | No Ct | No Ct | 36.4 | 32.2 | 33.5 |
| Amp_NTC | | | | | | | | | | |
| LDR_NTC | 34.2 | 44.8 | 35.7 | 37.9 | 37.1 | No Ct | 37.6 | 35.5 | 31.1 | 32.4 |
| TaqMan ™_NTC | No Ct | No Ct | No Ct | No Ct | No Ct | No Ct | No Ct | No Ct | No Ct | No Ct |

| | 11 | 12 | 13 | 14 | 15 | 16 | 17 | 18 | 19 | 20 |
|---|---|---|---|---|---|---|---|---|---|---|
| | Gene | | | | | | | | | |
| | VIM-S1 | CLIP4-S1 | GSG1L-S1 | PP1R16B-S1 | KCNA3 | GDF6-S1 | ADHFE1-S1 | THBD-S1 | SEPT9-S4 | SEMA3B-S1 |
| starting | 5001 | 5021 | 5051 | 5061 | 5071 | 5081 | 5101 | 5331 | 5351 | 5401 |
| number | | | | | | | | | | |
| for primer | | | | | | | | | | |
| 1,000 GE of | 10.4 | 5.7 | 7.7 | 17.9 | 11.2 | 14.2 | 17.5 | 30.0 | 7.2 | 9.2 |
| HT29 DNA + | | | | | | | | | | |
| 7,500 GE of | | | | | | | | | | |
| Roche DNA | | | | | | | | | | |
| 7,500 GE of | 17.6 | No Ct | 36.6 | 36.5 | 36.8 | 32.7 | 36.7 | No Ct | 31.2 | No Ct |
| Roche DNA | | | | | | | | | | |
| 1st Linaer | No Ct | No Ct | 36.8 | 36.3 | No Ct | 32.8 | 35.1 | No Ct | 31.5 | 41.4 |
| Amp_NTC | | | | | | | | | | |
| 2nd Linear | No Ct | No Ct | No Ct | 35.1 | No Ct | 33.3 | 36.8 | No Ct | 31.2 | No Ct |
| Amp_NTC | | | | | | | | | | |
| LDR_NTC | No Ct | No Ct | No Ct | 36.7 | No Ct | 32.6 | 38.3 | No Ct | 30.7 | 39.8 |
| TaqMan ™_NTC | No Ct | No Ct | No Ct | No Ct | No Ct | No Ct | No Ct | No Ct | No Ct | No Ct |

### Example 6: Multiplexed Detection of 20 CRCM Markers Using ex-PCR-LDR-qPCR on Bisulfite Converted HT29 Cell Line DNA

General Methods:

HT-29 colon adenocarcinoma cells were seeded in 60 cm2 culture dishes in McCoy's 5A medium containing 4.5 g/l glucose, supplemented with 10% fetal calf serum, and kept in a humidified atmosphere containing 5% CO₂. Once cells reached 80-90% confluence, they were washed in Phosphate Buffered Saline (×3), and collected by centrifugation (500×g) Genomic DNA was isolated using the DNeasy Blood & Tissue Kit frpm QIAGEN (Qiagen, Valencia, Calif.), and its concentration was measured using Quant-iT Pico green ds DNA Assay kit (Thermo-Fisher, Waltham, Mass.). High molecular weight (>50 kb) genomic DNA (0.2 mg/ml) isolated from normal human blood (buffy coat) (Roche human genomic DNA) was purchased from Roche (Indianapolis, Ind.). Its concentration was similarly determined using Quant-iT PicoGreen dsDNA Assay Kit (Thermo-Fisher, Waltham, Mass.). 1.0 μg HT29 cell line genomic DNA or Roche Normal DNA was fragmented through non random sonication method, using Covaris ultra sonicator E220 (Covaris, Woburn, Mass.). After shearing, the quality of the resulting DNA fragments (length ranged from 50 to 1 kb base pairs) was assessed with Agilent Bioanalyzer system 2100 (Agilent, Santa Clara, Calif.).

Enrichment of Methylated DNA:

The DNA fragments containing methylated CpGs was captured by methylation-specific antibodies, using the EpiMark® Methylated DNA Enrichment Kit from New England Biolabs, according to manufacturer's instructions (New England Biolabs, Ipswich, Mass.). DNA fragments containing methylated CpG sites were enriched by binding to the antibodies containing methyl-CpG binding domain. After a series of wash steps followed by magnetic capture, the enriched DNA sample was eluted in a small volume of water by incubation at 65° C.

Bisulfite Conversion of DNA:

Bisulfite conversion of cytosine bases in DNA was then carried out using the Cells-to-CpG Bisulfite Conversion kit from Applied Biosystem division of ThermoFisher (ThermoFisher, Carlsbad, Calif.). 5 μl of Denaturation Reagent was added to 45 μl of methyl enriched genomic DNA, followed by the mixture's incubation at 50° C. for 10 min. After adding 100 μl of Conversion Reagent, the mixture was incubated in a thermal cycler with the following program: 65° C. 30 min, 90° C. 30 sec, 65° C. 30 min, 90° C. 30 sec, 65° C. 30 min. 150 μl of converted DNA mixture was mixed with 600 μl of binding buffer in the binding Column. The column was centrifuged at 10,000 rpm for 1 min, followed by discarding the flow through. The column was washed with 600 μl of washing buffer. 200 μl of Desulfonation Reagent was added to the column, followed by incubation at room temperature for 15 min. After spinning, the column was washed again with 400 μl of washing buffer. 50 μl of Elution Buffer was then added to the column to elute the bound DNA. The mostly single stranded, bisulfite converted DNA was quantified with both Quant-iT Oli Green ss DNA kit and Pico Green ds DNA kit (ThermoFisher, Waltham, Mass.).

PCR Primers, LDR Probes, and LNA or PNA Blocking Primers:

All primers used are listed in Table 45 above. All primers were purchased from Integrated DNA Technologies Inc. (IDT) (Coralville, Iowa). All primers used in the preceding example were purchased from Integrated DNA Technologies Inc. (IDT; Coralville, Iowa). The primer number ended with 02A has the same sequences of the primer end with 02, but it has no short (10-mer) 5′ end tail sequences.

Templates Preparation:

Template “A”: 1 ug of sonicated and methylation enriched Roche Normal DNA was mixed with 6.6 ng of sonicated HT29 DNA in 50 μl, 10 ng human genomic DNA equals to 3,000 genomic equivalent (GE). The yield of methylation enrichment is about 50%, and the bisulfite conversion is about 50%. So the template A contains 75,000 GE of Roche DNA and 2,000 GE of HT29 DNA in 50 μl. 5 ul of DNA template A was used in the linear amplification reaction, it contains 7,500 GE of Roche DNA and 200 GE of HT29 DNA, Template “B”: 1 μg of sonicated and methylation enriched Roche Normal DNA was bisulfite converted and it has 75,000 GE of Roche DNA in 50 μl.

In the following experiment, 5 μl of HT29 DNA will be used, and it contains 1,000 GE of HT29 and 7,500 GE of Roche DNA. 5 μl of DNA template B was used in the linear amplification reaction, it contains 7,500 GE of Roche normal DNA.

Linear Amplification Step: For condition A, In a 25 μl of reaction volume, the linear amplification step was performed by mixing: 5 μl of 5× Gotaq Flexi buffer (no Magnesium) (Promega, Madison, Wis.), 3.5 μl of 25 mM MgCl2 (Promega, Madison, Wis.), 0.5 μl of 10 mM dNTPs (dATP, dCTP, dGTP and dTTP) (Promega, Madison, Wis.), 2.5 μl of second set of 20 plex marker-specific reverse primers with 10-mer short tail (concentration of each primer is 0.5 μM, primer number ends with 02 has a short tail, a universal sequences), 0.5 μl of tween 20 (5%), 0.9 μl of 20 mU/μl RNAseH2 (diluted in RNAseH2 dilution buffer from IDT) (IDT), and 0.5 μl of Klentaql polymerase (50 U/μl)(DNA Polymerase Technology, St. Louis, Mo.) mixed with Platinum Taq Antibody (Invitrogen/Thermo Fisher, Waltham, Mass.) (The ratio of mixing of Klentaql polymerase with Antibody is 1:10, and the final concentration of Klentaql polymerase is 5 U/μl), and 5.0 μl of corresponding bisulfite converted methylated enriched genomic DNA (out of 50 μl of eluted DNA after bisulfite conversion.). For condition B, in the other 25 ul of reaction mixture, all the reagents were the same except 2.5 μl of second set of 20 plex marker-specific reverse primers with no tail (Primer number ending with 02A). The template was either: 1) 5 ul of template A it contains 200 GE of HT29 DNA and 7,500 GE of Roche DNA. 2) 5 ul of template B, it contains 7,500 GE of Roche DNA. The reactions were run in a ProFlex PCR system thermocycler (Applied Biosystems/ThermoFisher, Waltham, Mass.) using the following program: 2 min at 94° C., 40 cycles of (20 sec at 94° C., 40 sec at 60° C., and 30 sec at 72° C.), and a final hold at 4° C. After the reaction, 0.5 μl of Platinum Taq antibodies were added in the reaction mixture to inhibit the Klentaq DNA polymerase.

PCR Reaction:

The linear amplification products were equally divided into two parts with two 10-plex reaction being carried out. In the 1st part, first 10 plex (marker number 21 to number 30) marker-specific forward primers and other reagents were added, in the 2nd part, the second 10 plex (marker number 31 to number 40) marker specific forward primers and other reagents were added. The PCR step was performed in a 20 μl of reaction mixture prepared by adding: 2 μl of Gotaq Flexi buffer 5× without Magnesium (Promega, Madison, Wis.), 1.4 μl of MgCl₂ at 25 mM (Promega, Madison, Wis.), 0.4 μl of dNTPs (with dATP, dCTP, dGTP and dUTP, 10 mM each) (Promega, Madison, Wis.), 0.2 μl of tween 20 (5%), 2 μl of 10 plex (1st 10-plex and 2nd 10-plex) marker-specific forward primers at 0.25 μM each. 0.4 μl of Antarctic Thermolabile UDG (1 u/μl)(New England Biolab, Ipswich, Mass.), 0.29 μl of RNAseH2 (IDT) at 20 mU/μl, 1.6 μl of Klentaql polymerase (DNA Polymerase Technology, St. Louis, Mo.) mixed with Platinum Taq Antibody (Invitrogen/Thermo Fisher, Waltham, Mass.) (The ratio of mixing of Klentaql polymerase with Antibody is 1:10, and the final concentration of Klentaql polymerase is 5 U/μl), 10 μl of corresponding linear amplification products and 1.7 μl water. PCR reactions were run in a ProFlex PCR system thermocycler (Applied Biosystems/ThermoFisher, Waltham, Mass.) and using the following program: 10 min at 37° C., 50 cycles of (94° C. 10 s, 60° C. 30 s, 72° C. 20 s), 10 min at 99.5° C., and a final hold at 4° C.

LDR Step:

The LDR step was performed in a 20 μl of reaction mixture prepared by combining: 11.6 μl of nuclease free water (IDT), 2 μl of 10×AK16D ligase reaction buffer [1× buffer contains 20 mM Tris-HCl pH 8.5 (Bio-Rad, Hercules, Calif.), 5 mM MgCl₂ (Sigma-Aldrich, St. Louis, Mo.), 50 mM KCl (Sigma-Aldrich, St. Louis, Mo.), 10 mM DTT (Sigma-Aldrich, St. Louis, Mo.) and 20 μg/ml of BSA (Sigma Aldrich, St. Louis, Mo.)], 0.5 μl of 40 mM DTT (Sigma-Aldrich, St. Louis, Mo.), 0.4 μl of 50 mM NAD+ (Sigma-Aldrich, St. Louis, Mo.), 0.5 μl of 20 mU/μl RNAseH2 (IDT), 0.4 μl of corresponding 10 plex LDR upstream probes at 500 nM each, 0.4 μl of corresponding 10 plex LDR downstream probes at 500 nM each, 0.57 μl of purified AK16D ligase (at 0.88 μM), and 4 μl of PCR reaction products from previous step. LDR reactions were run in a ProFlex PCR system thermocycler (Applied Biosystems/Thermo-Fisher; Waltham, Mass.) using the following program: 20 cycles of (10 sec at 94° C., and 4 min at 60° C.) followed by a final hold at 4° C.

qPCR Step:

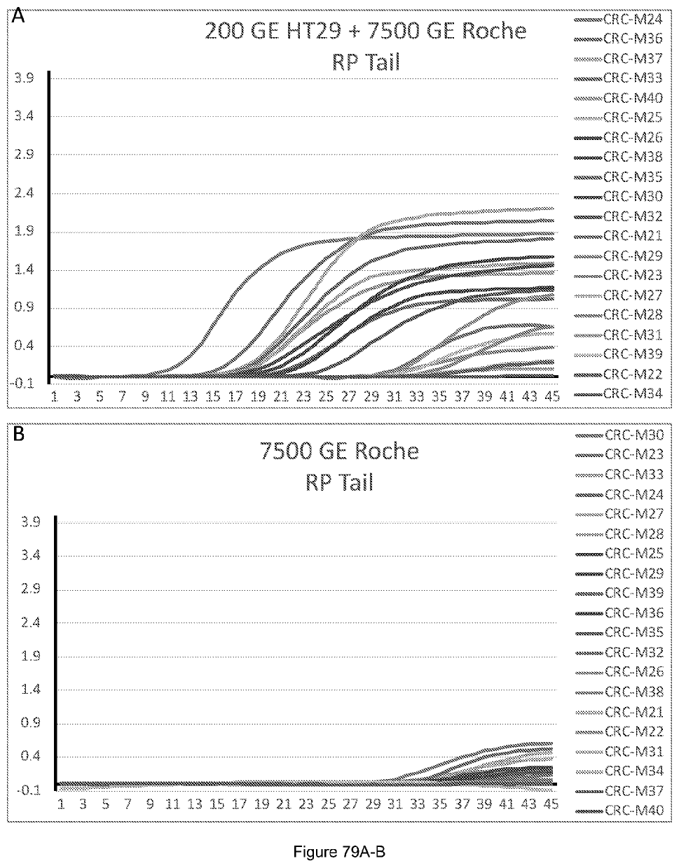

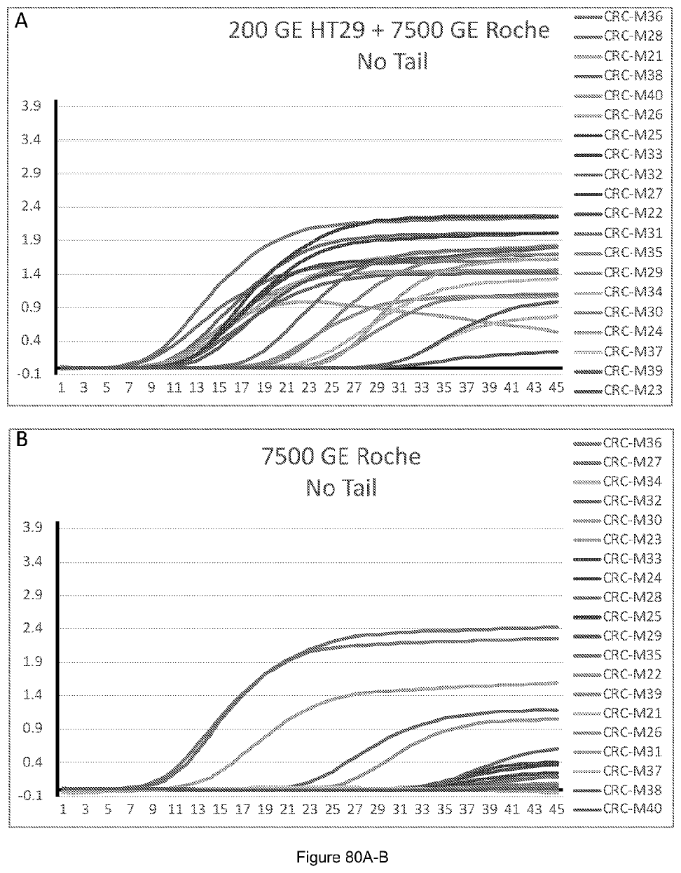

The qPCR reaction is run uni-plex with the qPCR step performed in a 10 μl of reaction volume by combining: 3 μl of nuclease free water (IDT), 5 μl of 2×TaqMan® Fast Universal PCR Master Mix (Fast amplitaq, UDG and dUTP) from Applied Biosystems (Applied Biosystems/ThermoFisher, Waltham, Mass.), 0.5 μl of a mixture of 5 μM TaqMan™ Assay one marker-specific forward primer and 5 μM of Taqman™ corresponding marker-specific reverse primer, 0.5 μl of 5 μM Taqman™ one marker-specific probe, and 1 μl of LDR 10-plex reaction products. qPCR reactions were carried out in a ViiA7 real-time thermo-cycler from Applied Biosystems (Applied Biosystems/Thermo-Fisher; Waltham, Mass.), using MicroAmp® Fast-96-Well Reaction 0.1 ml plates sealed with MicroAmp™ Optical adhesive film (Applied Biosystems/ThermoFisher; Waltham, Mass.), with the following setting: fast block, Standard curve as experiment type, ROX as passive reference, Ct as quantification method (automatic threshold, but adjusted to 0.05 when needed), TAMRA as reporter, and NFQ-MGB as quencher. The program employed was: 2 min at 50° C., and 45 cycles of (1 sec at 95° C., and 20 sec at 60° C.). Results are shown in FIGS. 79 and 80 and Table 62 below.

TABLE 62
Ct values for each gene in Example 6.

| | | CRC-Marker | | | | | | | | | |
|---|---|---|---|---|---|---|---|---|---|---|---|
| | | 1 | 2 | 3 | 4 | 5 | 6 | 7 | 8 | 9 | 10 |
| | | Gene | | | | | | | | | |
| | | IRF4 | TWIST1 | TDH | NCAM1 | COL4A2/1 | VSX1 | PTPRT | MIR124-3 | ZNF677 | EVC |
| Starting | RP02 | 5561 | 5571 | 5591 | 5621 | 5651 | 5661 | 5671 | 5681 | 5691 | 5701 |
| number | Status | | | | | | | | | | |
| for primer | | | | | | | | | | | |
| 200GE of | RP02 | 30.2 | 35.9 | 31.0 | 10.3 | 17.1 | 17.7 | 31.6 | 33.6 | 30.4 | 21.2 |
| HT29 + | with | | | | | | | | | | |
| 7,500 GE | Short | | | | | | | | | | |
| of Roche | Tail | | | | | | | | | | |
| 7,500 GE of | | No Ct | No Ct | 32.3 | 32.8 | 34.5 | 40.3 | 33.9 | 33.9 | 35.8 | 30.2 |
| Roche DNA | | | | | | | | | | | |
| 1st Linear | | No Ct | 39.0 | 32.5 | 33.2 | 35.5 | No Ct | 33.1 | 33.1 | 35.2 | 30.6 |
| Amplification_NTC | | | | | | | | | | | |
| 2nd | | No Ct | 37.5 | 32.3 | 33.1 | 34.2 | No Ct | 32.4 | 33.3 | 37.0 | 31.0 |
| PCR_NTC | | | | | | | | | | | |
| 200GE of | RP02A | 9.1 | 11.8 | 32.6 | 23.5 | 10.7 | 9.7 | 10.9 | 7.3 | 19.1 | 23.0 |
| HT29 + | without | | | | | | | | | | |
| 7,500 GE | Tail | | | | | | | | | | |
| of Roche | | | | | | | | | | | |
| 7,500 GE | | No Ct | 38.5 | 32.6 | 33.1 | 34.7 | No Ct | 8.3 | 33.2 | 36.3 | 24.9 |
| of Roche DNA | | | | | | | | | | | |
| 1st Linear | | No Ct | No Ct | 32.5 | 32.6 | 36.3 | No Ct | 32.8 | 33.8 | 34.8 | 30.8 |
| Amplification_NTC | | | | | | | | | | | |
| 2nd | | No Ct | 35.7 | 32.2 | 33.6 | 35.4 | No Ct | 33.1 | 32.9 | 37.0 | 31.2 |
| PCR_NTC | | | | | | | | | | | |

| | | CRC-Marker | | | | | | | | | |
|---|---|---|---|---|---|---|---|---|---|---|---|
| | | 11 | 12 | 13 | 14 | 15 | 16 | 17 | 18 | 19 | 20 |
| | | Gene | | | | | | | | | |
| | | | | | | | | | | SLC18A3, | |
| | | JAM2 | GFRA1 | FLI1 | GNAO1 | CoCaNCR2 | CoCaNCR9 | CoCaNCR10 | CoCaNCR8 | CHAT | LIFR |
| starting | RP02 | 5711 | 5721 | 5731 | 5741 | 5751 | 5761 | 5771 | 5781 | 5801 | 5811 |
| number | Status | | | | | | | | | | |
| for | | | | | | | | | | | |
| primer | | | | | | | | | | | |
| 200GE of | RP02 | 35.0 | 23.8 | 16.5 | No Ct | 20.5 | 14.4 | 16.1 | 19.1 | 35.8 | 16.9 |
| HT29 + | With | | | | | | | | | | |
| 7,500 GE | Short | | | | | | | | | | |
| of Roche | Tail | | | | | | | | | | |
| 7,500 GE of | | No Ct | 38.4 | 32.7 | No Ct | 38.2 | 36.0 | No Ct | 41.2 | 35.9 | No Ct |
| Roche DNA | | | | | | | | | | | |
| 1st Linear | | No Ct | 38.6 | 32.4 | No Ct | 41.6 | 35.6 | No Ct | 38.9 | 35.5 | No Ct |
| Amplification_NTC | | | | | | | | | | | |
| 2nd | | No Ct | 38.0 | 32.0 | No Ct | 40.6 | 37.4 | No Ct | 35.7 | 36.1 | No Ct |
| PCR_NTC | | | | | | | | | | | |
| 200GE of | RP02A | 16.1 | 10.9 | 10.7 | 21.5 | 18.2 | 6.9 | 29.7 | 9.2 | 29.9 | 9.5 |
| HT29 + | Without | | | | | | | | | | |
| 7,500 GE | Tail | | | | | | | | | | |
| of Roche | | | | | | | | | | | |
| 7,500 GE of | | No Ct | 21.6 | 32.7 | 11.7 | 37.8 | 7.8 | No Ct | No Ct | 41.5 | No Ct |
| Roche DNA | | | | | | | | | | | |
| 1st Linear | | No Ct | 39.2 | 32.6 | No Ct | 39.7 | 39.4 | No Ct | No Ct | 35.4 | No Ct |
| Amplification_NTC | | | | | | | | | | | |
| 2nd | | No Ct | 35.3 | 32.8 | No Ct | 37.9 | 38.3 | No Ct | 37.3 | 36.6 | No Ct |
| PCR_NTC | | | | | | | | | | | |

Although preferred embodiments have been depicted and described in detail herein, it will be apparent to those skilled in the relevant art that various modifications, additions, substitutions, and the like can be made without departing from the spirit of the present application and these are therefore considered to be within the scope of the present application as defined in the claims which follow.

LENGTHY TABLES
The patent application contains a lengthy table section. A copy of the table is available in electronic form from the USPTO web site (https://seqdata.uspto.gov/?pageRequest=docDetail&DocID=US20220243263A1). An electronic copy of the table will also be available from the USPTO upon request and payment of the fee set forth in 37 CFR 1.19(b)(3).

What is claimed:

1. A method for identifying, in a sample, one or more parent nucleic acid molecules containing a target nucleotide sequence differing from nucleotide sequences of other parent nucleic acid molecules in the sample by one or more nucleotides, one or more copy numbers, one or more transcript sequences, and/or one or more methylated residues, said method comprising:
providing a sample containing one or more parent nucleic acid molecules potentially containing the target nucleotide sequence differing from the nucleotide sequences of other parent nucleic acid molecules by one or more nucleotides, one or more copy numbers, one or more transcript sequences, and/or one or more methylated residues;
providing one or more enzymes capable of digesting deoxyuracil (dU)-containing nucleic acid molecules;
providing one or more primary oligonucleotide primer sets, each primary oligonucleotide primer set comprising (a) a first primary oligonucleotide primer that comprises a nucleotide sequence that is complementary to a sequence in the parent nucleic acid molecule adjacent to the target nucleotide sequence and (b) a second primary oligonucleotide primer that comprises a nucleotide sequence that is complementary to a portion of an extension product formed from the first primary oligonucleotide primer;
blending the sample, the one or more first primary oligonucleotide primers of the primary oligonucleotide primer sets, the one or more enzymes capable of digesting deoxyuracil (dU)-containing nucleic acid molecules, a deoxynucleotide mix, and a DNA polymerase to form one or more polymerase extension reaction mixtures;
subjecting the one or more polymerase extension reaction mixtures to conditions suitable for digesting deoxyuracil (dU)-containing nucleic acid molecules present in the polymerase extension reaction mixtures and for carrying out one or more polymerase extension reaction cycles comprising a denaturation treatment, a hybridization treatment, and an extension treatment, thereby forming primary extension products comprising nucleotide sequences complementary to the target nucleotide sequence;
blending the one or more polymerase extension reaction mixtures comprising the primary extension products, the one or more second primary oligonucleotide primers of the primary oligonucleotide primer sets, the one or more enzymes capable of digesting deoxyuracil (dU)-containing nucleic acid molecules in the reaction mixtures, a deoxynucleotide mix including dUTP, and a DNA polymerase to form one or more first polymerase chain reaction mixtures;
subjecting the one or more first polymerase chain reaction mixtures to conditions suitable for digesting deoxyuracil (dU)-containing nucleic acid molecules present in the polymerase chain reaction mixtures and for carrying out one or more polymerase chain reaction cycles comprising a denaturation treatment, a hybridization treatment, and an extension treatment, thereby forming one or more first polymerase chain reaction products comprising the target nucleotide sequence or a complement thereof;
providing one or more oligonucleotide probe sets, each probe set comprising (a) a first oligonucleotide probe having a 5′ primer-specific portion and a 3′ target sequence-specific portion, and (b) a second oligonucleotide probe having a 5′ target sequence-specific portion and a 3′ primer-specific portion, wherein the first and second oligonucleotide probes of a probe set are configured to hybridize, in a base specific manner, on a complementary target nucleotide sequence of a secondary extension product;
blending the one or more first polymerase chain reaction products with a ligase, and the one or more oligonucleotide probe sets to form one or more ligation reaction mixtures;
subjecting the one or more ligation reaction mixtures to one or more ligation reaction cycles whereby the first and second oligonucleotide probes of the one or more oligonucleotide probe sets are ligated together, when hybridized to their complementary sequence, to form ligated product sequences in the ligation reaction mixtures wherein each ligated product sequence comprises the 5′ primer-specific portion, the target-specific portions, and the 3′ primer-specific portion;
providing one or more secondary oligonucleotide primer sets, each secondary oligonucleotide primer set comprising (a) a first secondary oligonucleotide primer comprising the same nucleotide sequence as the 5′ primer-specific portion of the ligated product sequence and (b) a second secondary oligonucleotide primer comprising a nucleotide sequence that is complementary to the 3′ primer-specific portion of the ligated product sequence;
blending the ligated product sequences, the one or more secondary oligonucleotide primer sets, the one or more enzymes capable of digesting deoxyuracil (dU)-containing nucleic acid molecules, a deoxynucleotide mix including dUTP, and a DNA polymerase to form one or more second polymerase chain reaction mixtures;
subjecting the one or more second polymerase chain reaction mixtures to conditions suitable for digesting deoxyuracil (dU)-containing nucleic acid molecules present in the second polymerase chain reaction mixtures and for carrying out one or more polymerase chain reaction cycles comprising a denaturation treatment, a hybridization treatment, and an extension treatment thereby forming one or more second polymerase chain reaction products; and
detecting and distinguishing the one or more second polymerase chain reaction products in the one or more second polymerase chain reaction mixtures to identify the presence of one or more parent nucleic acid molecules containing target nucleotide sequences differing from nucleotide sequences in other parent nucleic acid molecules in the sample by one or more nucleotides, one or more copy numbers, one or more transcript sequences, and/or one or more methylated residues.

2. A method for identifying, in a sample, one or more parent nucleic acid molecules containing a target nucleotide sequence differing from nucleotide sequences in other parent nucleic acid molecules in the sample by one or more nucleotides, one or more copy numbers, one or more transcript sequences, and/or one or more methylated residues, said method comprising:
providing a sample containing one or more parent nucleic acid molecules potentially containing the target nucleotide sequence differing from the nucleotide sequences of other parent nucleic acid molecules by one or more nucleotides, one or more copy numbers, one or more transcript sequences, and/or one or more methylated residues;
providing one or more enzymes capable of digesting deoxyuracil (dU)-containing nucleic acid molecules;
providing one or more nucleases capable of digesting nucleic acid molecules not comprising modified nucleotides;
providing one or more first primary oligonucleotide primer(s) that comprises a nucleotide sequence that is complementary to a sequence in the parent nucleic acid molecule adjacent to the target nucleotide sequence;
blending the sample, the one or more first primary oligonucleotide primers, the one or more enzymes capable of digesting deoxyuracil (dU)-containing nucleic acid molecules, a deoxynucleotide mix that comprises one or more modified nucleotides that protect extension products but not target DNA from nuclease digestion, and a DNA polymerase to form one or more polymerase extension reaction mixtures;
subjecting the one or more polymerase extension reaction mixtures to conditions suitable for digesting deoxyuracil (dU)-containing nucleic acid molecules present in the polymerase extension reaction mixture and for carrying out one or more polymerase extension reaction cycles comprising a denaturation treatment, a hybridization treatment, and an extension treatment, thereby forming primary extension products comprising the complement of the target nucleotide sequence;
providing one or more secondary oligonucleotide primer sets, each secondary oligonucleotide primer set comprising (a) a first secondary oligonucleotide primer having a first 5′ primer-specific portion and a 3′ portion that is complementary to a portion of a primary extension product formed from the first primary oligonucleotide primer and (b) a second secondary oligonucleotide primer having a second 5′ primer-specific portion and a 3′ portion that comprises a nucleotide sequence that is complementary to a portion of an extension product formed from the first secondary oligonucleotide primer;
blending the one or more polymerase extension reaction mixtures comprising the primary extension products, the one or more secondary oligonucleotide primer sets, the one or more nucleases, a deoxynucleotide mix, and a DNA polymerase to form one or more first polymerase chain reaction mixtures;
subjecting the one or more first polymerase chain reaction mixtures to conditions suitable for digesting nucleic acid molecules present in the first polymerase chain reaction mixtures, but not primary extension products comprising modified nucleotides and for carrying out two or more polymerase chain reaction cycles comprising a denaturation treatment, a hybridization treatment, and an extension treatment, thereby forming one or more first polymerase chain reaction products comprising the first 5′ primer-specific portion, a target-specific nucleotide sequence or a complement thereof, and a complement of the second 5′ primer-specific portion;
providing one or more tertiary oligonucleotide primer sets, each tertiary oligonucleotide primer set comprising (a) a first tertiary oligonucleotide primer comprising the same nucleotide sequence as the first 5′ primer-specific portion of the one or more first polymerase chain reaction products and (b) a second tertiary oligonucleotide primer comprising a nucleotide sequence that is complementary to the 3′ primer-specific portion of the one or more first polymerase chain reaction products;
blending the one or more first polymerase chain reaction products, the one or more tertiary oligonucleotide primer sets, the one or more enzymes capable of digesting deoxyuracil (dU)-containing nucleic acid molecules, a deoxynucleotide mix including dUTP, and a DNA polymerase to form one or more second polymerase chain reaction mixtures;
subjecting the one or more second polymerase chain reaction mixtures to conditions suitable for digesting deoxyuracil (dU) containing nucleic acid molecules present in the second polymerase chain reaction mixtures and for carrying out one or more polymerase chain reaction cycles comprising a denaturation treatment, a hybridization treatment, and an extension treatment, thereby forming one or more second polymerase chain reaction products; and
detecting and distinguishing the one or more second polymerase chain reaction products in the one or more second polymerase chain reaction mixtures to identify the presence of one or more parent nucleic acid molecules containing target nucleotide sequences differing from nucleotide sequences in other parent nucleic acid molecules in the sample by one or more nucleotides, one or more copy numbers, one or more transcript sequences, and/or one or more methylated residues.

3. A method for identifying, in a sample, one or more parent nucleic acid molecules containing a target nucleotide sequence differing from nucleotide sequences of other parent nucleic acid molecules in the sample by one or more nucleotides, one or more copy numbers, one or more transcript sequences, and/or one or more methylated residues, said method comprising:
providing a sample containing one or more parent nucleic acid molecules potentially containing the target nucleotide sequence differing from the nucleotide sequences of other parent nucleic acid molecules by one or more nucleotides, one or more copy numbers, one or more transcript sequences, and/or one or more methylated residues;
providing one or more enzymes capable of digesting deoxyuracil (dU)-containing nucleic acid molecules;
providing one or more nucleases capable of digesting nucleic acid molecules present not comprising modified nucleotides;
providing one or more primary oligonucleotide primer sets, each primary oligonucleotide primer set comprising (a) a first primary oligonucleotide primer that comprises a nucleotide sequence that is complementary to a sequence in the parent nucleic acid molecule adjacent to the target nucleotide sequence and (b) a second primary oligonucleotide primer that comprises a nucleotide sequence that is complementary to a portion of an extension product formed from the first primary oligonucleotide primer;
blending the sample, the one or more first primary oligonucleotide primers of the primary oligonucleotide primer sets, the one or more enzymes capable of digesting deoxyuracil (dU)-containing nucleic acid molecules, a deoxynucleotide mix that comprises one or more modified nucleotides that protect extension product but not target DNA from nuclease digestion, and a DNA polymerase to form one or more polymerase extension reaction mixtures;
subjecting the one or more polymerase extension reaction mixtures to conditions suitable for digesting deoxyuracil (dU)-containing nucleic acid molecules present in the polymerase extension reaction mixtures and for carrying out one or more polymerase extension reaction cycles comprising a denaturation treatment, a hybridization treatment, and an extension treatment, thereby forming primary extension products comprising the complement of the target nucleotide sequence;
blending the one or more polymerase extension reaction mixtures comprising the primary extension products, the one or more second primary oligonucleotide primers of the one or more primary oligonucleotide primer sets, the one or more nucleases, a deoxynucleotide mix, and a DNA polymerase to form one or more first polymerase chain reaction mixtures;
subjecting the one or more first polymerase chain reaction mixtures to conditions suitable for digesting nucleic acid molecules present in the polymerase chain reaction mixtures, but not primary extension products comprising modified nucleotides and for carrying out two or more polymerase chain reaction cycles comprising a denaturation treatment, a hybridization treatment, and an extension treatment, thereby forming first polymerase chain reaction products comprising the target nucleotide sequence or a complement thereof;
providing one or more secondary oligonucleotide primer sets, each secondary oligonucleotide primer set comprising (a) a first secondary oligonucleotide primer having a 3′ portion that is complementary to a portion of an extension product formed from the first primary oligonucleotide primer and (b) a second secondary oligonucleotide primer having a 3′ portion that comprises a nucleotide sequence that is complementary to a portion of an extension product formed from the first secondary oligonucleotide primer;
blending the first polymerase chain reaction products, the one or more secondary oligonucleotide primer sets, the one or more enzymes capable of digesting deoxyuracil (dU) containing nucleic acid molecules, a deoxynucleotide mix including dUTP, and a DNA polymerase to form one or more second polymerase chain reaction mixtures;
subjecting the one or more second polymerase chain reaction mixtures to conditions suitable for digesting deoxyuracil (dU)-containing nucleic acid molecules present in the second polymerase chain reaction mixtures and for carrying out two or more polymerase chain reaction cycles comprising a denaturation treatment, a hybridization treatment, and an extension treatment thereby forming second polymerase chain reaction products; and
detecting and distinguishing the second polymerase chain reaction products in the one or more second polymerase chain reaction mixtures to identify the presence of one or more parent nucleic acid molecules containing target nucleotide sequences differing from nucleotide sequences in other parent nucleic acid molecules in the sample by one or more nucleotides, one or more copy numbers, one or more transcript sequences, and/or one or more methylated residues.

4. A method for identifying, in a sample, one or more parent nucleic acid molecules containing a target nucleotide sequence differing from nucleotide sequences in other parent nucleic acid molecules in the sample by one or more methylated residues, said method comprising:
providing a sample containing one or more parent nucleic acid molecules potentially containing the target nucleotide sequence differing from the nucleotide sequences in other parent nucleic acid molecules by one or more methylated residues;
subjecting the nucleic acid molecules in the sample to a bisulfite treatment under conditions suitable to convert unmethylated cytosine residues to uracil residues;
providing one or more enzymes capable of digesting deoxyuracil (dU)-containing nucleic acid molecules;
providing one or more primary oligonucleotide primer sets, each primary oligonucleotide primer set comprising (a) a first primary oligonucleotide primer that comprises a nucleotide sequence that is complementary to a sequence in the bisulfite-treated parent nucleic acid molecules adjacent to the bisulfite-treated target nucleotide sequence containing the one or more methylated residue and (b) a second primary oligonucleotide primer that comprises a nucleotide sequence that is complementary to a portion of an extension product formed from the first primary oligonucleotide primer;
blending the bisulfite-treated sample, the one or more first primary oligonucleotide primers of the one or more primary oligonucleotide primer sets, a deoxynucleotide mix, and a DNA polymerase to form one or more polymerase extension reaction mixtures;
subjecting the one or more polymerase extension reaction mixtures to conditions suitable for one or more polymerase extension reaction cycles comprising a denaturation treatment, a hybridization treatment, and an extension treatment, thereby forming primary extension products comprising the complement of the bisulfite-treated target nucleotide sequence;
blending the one or more polymerase extension reaction mixtures comprising the primary extension products, the one or more secondary primary oligonucleotide primers of the one or more primary oligonucleotide primer sets, the one or more enzymes capable of digesting deoxyuracil (dU)-containing nucleic acid molecules, a deoxynucleotide mix including dUTP, and a DNA polymerase to form one or more first polymerase chain reaction mixtures;
subjecting the one or more first polymerase chain reaction mixtures to conditions suitable for digesting deoxyuracil (dU)-containing nucleic acid molecules present in the first polymerase chain reaction mixtures and for carrying out one or more polymerase chain reaction cycles comprising a denaturation treatment, a hybridization treatment, and an extension treatment, thereby forming first polymerase chain reaction products comprising the bisulfate-treated target nucleotide sequence or a complement thereof;
providing one or more oligonucleotide probe sets, each probe set comprising (a) a first oligonucleotide probe having a 5′ primer-specific portion and a 3′ bisulfate-treated target nucleotide sequence-specific or complement sequence-specific portion, and (b) a second oligonucleotide probe having a 5′ bisulfate-treated target nucleotide sequence-specific or complement sequence-specific portion and a 3′ primer-specific portion, and wherein the first and second oligonucleotide probes of a probe set are configured to hybridize, in a base specific manner, on a complementary nucleotide sequence of a first polymerase chain reaction product;
blending the first polymerase chain reaction products with a ligase and the one or more oligonucleotide probe sets to form one or more ligation reaction mixtures;
subjecting the one or more ligation reaction mixtures to one or more ligation reaction cycles whereby the first and second oligonucleotide probes of the one or more oligonucleotide probe sets are ligated together, when hybridized to complementary sequences, to form ligated product sequences in the ligation reaction mixture wherein each ligated product sequence comprises the 5′ primer-specific portion, the bisulfite-treated target nucleotide sequence-specific or complement sequence-specific portions, and the 3′ primer-specific portion;
providing one or more secondary oligonucleotide primer sets, each secondary oligonucleotide primer set comprising (a) a first secondary oligonucleotide primer comprising the same nucleotide sequence as the 5′ primer-specific portion of the ligated product sequence and (b) a second secondary oligonucleotide primer comprising a nucleotide sequence that is complementary to the 3′ primer-specific portion of the ligated product sequence;
blending the ligated product sequences, the one or more secondary oligonucleotide primer sets, the one or more enzymes capable of digesting deoxyuracil (dU)-containing nucleic acid molecules, a deoxynucleotide mix including dUTP, and a DNA polymerase to form one or more second polymerase chain reaction mixtures;
subjecting the one or more second polymerase chain reaction mixtures to conditions suitable for digesting deoxyuracil (dU)-containing nucleic acid molecules present in the second polymerase chain reaction mixtures and for carrying out one or more polymerase chain reaction cycles comprising a denaturation treatment, a hybridization treatment, and an extension treatment thereby forming a second polymerase chain reaction products; and
detecting and distinguishing the second polymerase chain reaction products in the one or more second polymerase chain reaction mixtures to identify the presence of one or more nucleic acid molecules containing target nucleotide sequences differing from nucleotide sequences in other parent nucleic acid molecules in the sample by one or more methylated residues.

5. A method for identifying, in a sample, one or more parent nucleic acid molecules containing a target nucleotide sequence differing from nucleotide sequences in other parent nucleic acid molecules in the sample by one or more methylated residues, said method comprising:
providing a sample containing one or more parent nucleic acid molecules potentially containing the target nucleotide sequence differing from the nucleotide sequences in other parent nucleic acid molecules by one or more methylated residues;
subjecting the nucleic acid molecules in the sample to a bisulfite treatment under conditions suitable to convert unmethylated cytosine residues to uracil residues;
providing one or more enzymes capable of digesting deoxyuracil (dU)-containing nucleic acid molecules;
providing one or more first primary oligonucleotide primer(s) that comprises a nucleotide sequence that is complementary to a sequence in the bisulfite-treated parent nucleic acid molecules adjacent to the bisulfite-treated target nucleotide sequence containing the one or more methylated residue;
blending the bisulfite-treated sample, the one or more first primary oligonucleotide primers, a deoxynucleotide mix, and a DNA polymerase to form one or more polymerase extension reaction mixtures;
subjecting the one or more polymerase extension reaction mixtures to conditions suitable for one or more polymerase extension reaction cycles comprising a denaturation treatment, a hybridization treatment, and an extension treatment, thereby forming primary extension products comprising the complement of the bisulfite-treated target nucleotide sequence;
providing one or more secondary oligonucleotide primer sets, each secondary oligonucleotide primer set comprising (a) a first secondary oligonucleotide primer having a 5′ primer-specific portion and a 3′ portion that is complementary to a portion of the polymerase extension reaction product formed from the first primary oligonucleotide primer and (b) a second secondary oligonucleotide primer having a 5′ primer-specific portion and a 3′ portion that comprises a nucleotide sequence that is complementary to a portion of an extension product formed from the first secondary oligonucleotide primer;
blending the one or more polymerase extension reaction mixtures comprising the primary extension products, the one or more secondary oligonucleotide primer sets, the one or more enzymes capable of digesting deoxyuracil (dU)-containing nucleic acid molecules, a deoxynucleotide mix, and a DNA polymerase to form one or more first polymerase chain reaction mixtures;
subjecting the one or more first polymerase chain reaction mixtures to conditions suitable for digesting nucleic acid molecules present in the first polymerase chain reaction mixtures, but not primary extension products comprising modified nucleotides and for carrying out two or more polymerase chain reaction cycles comprising a denaturation treatment, a hybridization treatment, and an extension treatment, thereby forming first polymerase chain reactions products comprising a 5′ primer-specific portion of the first secondary oligonucleotide primer, the bisulfite-treated target nucleotide sequence-specific or complement sequence-specific portion, and a complement of the 5′ primer-specific portion of the second secondary oligonucleotide primer;
providing one or more tertiary oligonucleotide primer sets, each tertiary oligonucleotide primer set comprising (a) a first tertiary oligonucleotide primer comprising the same nucleotide sequence as the 5′ primer-specific portion of the first polymerase chain reaction products and (b) a second tertiary oligonucleotide primer comprising a nucleotide sequence that is complementary to the 3′ primer-specific portion of the first polymerase chain reactions product sequence;
blending the first polymerase chain reaction products, the one or more tertiary oligonucleotide primer sets, the one or more enzymes capable of digesting deoxyuracil (dU) containing nucleic acid molecules, a deoxynucleotide mix including dUTP, and a DNA polymerase to form one or more second polymerase chain reaction mixtures;
subjecting the one or more second polymerase chain reaction mixtures to conditions suitable for digesting deoxyuracil (dU)-containing nucleic acid molecules present in the second polymerase chain reaction mixtures and for carrying out one or more polymerase chain reaction cycles comprising a denaturation treatment, a hybridization treatment, and an extension treatment thereby forming secondary polymerase chain reaction products; and
detecting and distinguishing the secondary polymerase chain reactions products in the one or more second polymerase chain reaction mixtures to identify the presence of one or more parent nucleic acid molecules containing target nucleotide sequences differing from nucleotide sequences in other parent nucleic acid molecules in the sample by one or more methylated residues.

6. A method for identifying, in a sample, one or more parent nucleic acid molecules containing a target nucleotide sequence differing from nucleotide sequences in other parent nucleic acid molecules in the sample by one or more methylated residues, said method comprising:
providing a sample containing one or more parent nucleic acid molecules potentially containing the target nucleotide sequence differing from the nucleotide sequences in other parent nucleic acid molecules by one or more methylated residues;
subjecting the nucleic acid molecules in the sample to a bisulfite treatment under conditions suitable to convert unmethylated cytosine residues to uracil residues;
providing one or more enzymes capable of digesting deoxyuracil (dU)-containing nucleic acid molecules present in the sample;
providing one or more primary oligonucleotide primer sets, each primary oligonucleotide primer set comprising (a) a first primary oligonucleotide primer that comprises a nucleotide sequence that is complementary to a sequence in the bisulfite-treated parent nucleic acid molecules adjacent to the bisulfite-treated target nucleotide sequence containing the one or more methylated residue and (b) a second primary oligonucleotide primer that comprises a nucleotide sequence that is complementary to a portion of an extension product formed from the first primary oligonucleotide primer;
blending the bisulfite-treated sample, the one or more first primary oligonucleotide primers of the one or more primary oligonucleotide primer sets, a deoxynucleotide mix, and a DNA polymerase to form one or more polymerase extension reaction mixtures;
subjecting the one or more polymerase extension reaction mixtures to conditions suitable for one or more polymerase extension reaction cycles comprising a denaturation treatment, a hybridization treatment, and an extension treatment, thereby forming primary extension products comprising the complement of the bisulfite treated target nucleotide sequence;
blending the one or more polymerase extension reaction mixtures comprising the primary extension products, the one or more secondary primary oligonucleotide primers of the one or more primary oligonucleotide primer sets, the one or more enzymes capable of digesting deoxyuracil (dU)-containing nucleic acid molecules in the reaction mixture, a deoxynucleotide mix, and a DNA polymerase to form one or more first polymerase chain reaction mixtures;
subjecting the one or more first polymerase chain reaction mixtures to conditions suitable for digesting deoxyuracil (dU)-containing nucleic acid molecules present in the first polymerase chain reaction mixtures and for carrying out one or more polymerase chain reaction cycles comprising a denaturation treatment, a hybridization treatment, and an extension treatment, thereby forming first polymerase chain reaction products comprising the bisulfite-treated target nucleotide sequence or a complement thereof;
providing one or more secondary oligonucleotide primer sets, each secondary oligonucleotide primer set comprising (a) a first secondary oligonucleotide primer having a 3′ portion that is complementary to a portion of a first polymerase chain reaction product formed from the first primary oligonucleotide primer and (b) a second secondary oligonucleotide primer having a 3′ portion that comprises a nucleotide sequence that is complementary to a portion of a first polymerase chain reaction product formed from the first secondary oligonucleotide primer;
blending the first polymerase chain reaction products, the one or more secondary oligonucleotide primer sets, the one or more enzymes capable of digesting deoxyuracil (dU)-containing nucleic acid molecules, a deoxynucleotide mix including dUTP, and a DNA polymerase to form one or more second polymerase chain reaction mixtures;
subjecting the one or more second polymerase chain reaction mixtures to conditions suitable for digesting deoxyuracil (dU)-containing nucleic acid molecules present in the second polymerase chain reaction mixtures and for carrying out two or more polymerase chain reaction cycles comprising a denaturation treatment, a hybridization treatment, and an extension treatment thereby forming second polymerase chain reaction products; and
detecting and distinguishing the second polymerase chain reactions products in the one or more second polymerase chain reaction mixtures to identify the presence of one or more parent nucleic acid molecules containing target nucleotide sequences differing from nucleotide sequences in other parent nucleic acid molecules in the sample by one or more methylated residues.

7. A method for identifying, in a sample, one or more parent nucleic acid molecules containing a target nucleotide sequence differing from nucleotide sequences in other parent nucleic acid molecules in the sample by one or more methylated residues, said method comprising:
providing a sample containing one or more parent nucleic acid molecules potentially containing the target nucleotide sequence differing from the nucleotide sequences in other parent nucleic acid molecules by one or more methylated residues;
subjecting the nucleic acid molecules in the sample to a bisulfite treatment under conditions suitable to convert unmethylated cytosine residues to uracil residues;
providing one or more enzymes capable of digesting deoxyuracil (dU)-containing nucleic acid molecules present in the sample;
providing one or more primary oligonucleotide primer sets, each primary oligonucleotide primer set comprising (a) a first primary oligonucleotide primer having a 5′ primer-specific portion and a 3′ portion that comprises a nucleotide sequence that is complementary to a sequence in the bisulfite-treated parent nucleic acid molecules adjacent to the bisulfite-treated target nucleotide sequence containing the one or more methylated residue and (b) a second primary oligonucleotide primer having a 5′ primer-specific portion and a 3′ portion that comprises a nucleotide sequence that is complementary to a portion of an extension product formed from the first primary oligonucleotide primer;
blending the bisulfite treated sample, the one or more first primary oligonucleotide primers of the one or more primary oligonucleotide primer sets, a deoxynucleotide mix, and a DNA polymerase to form one or more polymerase extension reaction mixtures;
subjecting the one or more polymerase extension reaction mixtures to conditions suitable for one or more polymerase extension reaction cycles comprising a denaturation treatment, a hybridization treatment, and an extension treatment, thereby forming primary extension products comprising the complement of the bisulfite treated target nucleotide sequence;
blending the one or more polymerase extension reaction mixtures comprising the primary extension products, the one or more secondary primary oligonucleotide primers of the one or more primary oligonucleotide primer sets, the one or more enzymes capable of digesting deoxyuracil (dU)-containing nucleic acid molecules in the reaction mixture, a deoxynucleotide mix, and a DNA polymerase to form one or more first polymerase chain reaction mixtures;
subjecting the one or more first polymerase chain reaction mixtures to conditions suitable for digesting deoxyuracil (dU)-containing nucleic acid molecules present in the polymerase chain reaction mixtures and for carrying out one or more polymerase chain reaction cycles comprising a denaturation treatment, a hybridization treatment, and an extension treatment, thereby forming first polymerase chain reactions products comprising the bisulfite-treated target nucleotide sequence or a complement thereof;
providing one or more secondary oligonucleotide primer sets, each secondary oligonucleotide primer set comprising (a) a first secondary oligonucleotide primer comprising the same nucleotide sequence as the 5′ primer-specific portion of the first polymerase chain reaction products or their complements and (b) a second secondary oligonucleotide primer comprising a nucleotide sequence that is complementary to the 3′ primer-specific portion of the first polymerase chain reaction products or their complements;
blending the primary polymerase chain reaction product sequences, the one or more secondary oligonucleotide primer sets, the one or more enzymes capable of digesting deoxyuracil (dU)-containing nucleic acid molecules, a deoxynucleotide mix including dUTP, and a DNA polymerase to form one or more second polymerase chain reaction mixtures;
subjecting the one or more second polymerase chain reaction mixtures to conditions suitable for digesting deoxyuracil (dU)-containing nucleic acid molecules present in the second polymerase chain reaction mixtures and for carrying out one or more polymerase chain reaction cycles comprising a denaturation treatment, a hybridization treatment, and an extension treatment thereby forming second polymerase chain reaction products; and
detecting and distinguishing the second polymerase chain reactions products in the one or more second polymerase chain reaction mixtures to identify the presence of one or more parent nucleic acid molecules containing target nucleotide sequences differing from nucleotide sequences in other parent nucleic acid molecules in the sample by one or more methylated residues.

8. The method of any one of claims 1 through 7 further comprising:
contacting the sample with DNA repair enzymes to repair damaged DNA, abasic sites, oxidized bases, or nicks in the DNA.

9. The method of any one of claims 4 through 7 further comprising:
contacting the sample with at least a first methylation sensitive enzyme to form a restriction enzyme reaction mixture prior to, or concurrent with, said blending to form one or more polymerase extension reaction mixtures, wherein said first methylation sensitive enzyme cleaves nucleic acid molecules in the sample that contain one or more unmethylated residues within at least one methylation sensitive enzyme recognition sequence, and whereby said detecting involves detection of one or more parent nucleic acid molecules containing the target nucleotide sequence, wherein said parent nucleic acid molecules originally contained one or more methylated residues.

10. The method of any one of claims 4 through 7 further comprising:
contacting the sample with an immobilized methylated nucleic acid binding protein or antibody to selectively bind and enrich for methylated nucleic acid in the sample.

11. The method of any one of claims 1 through 7, wherein primers from said one or more primary or secondary oligonucleotide primer sets comprise a portion that has no or one nucleotide sequence mismatch when hybridized in a base-specific manner to the target nucleic acid sequence or bisulfite-converted methylated nucleic acid sequence or complement sequence thereof, but have one or more additional nucleotide sequence mismatches that interferes with polymerase extension when primers from said one or more primary or secondary oligonucleotide primer sets hybridize in a base-specific manner to a corresponding nucleotide sequence portion in wildtype nucleic acid sequence or bisulfite-converted unmethylated nucleic acid sequence or complement sequence thereof.

12. The method of any one of claims 1 through 7, wherein one or both primary oligonucleotide primers of the primary oligonucleotide primer set and/or one or both secondary oligonucleotide primers of the secondary oligonucleotide primer set have a 3′ portion comprising a cleavable nucleotide or nucleotide analogue and a blocking group, such that the 3′ end of said primer or primers is unsuitable for polymerase extension, said method further comprising:
cleaving the cleavable nucleotide or nucleotide analog of one or both oligonucleotide primers during said hybridization treatment, thereby liberating free 3′OH ends on one or both oligonucleotide primers prior to said extension treatment.

13. The method of claim 12, wherein primers from said one or more primary or secondary oligonucleotide primer sets comprise a sequence that differs from the target nucleic acid sequence or bisulfite-converted methylated nucleic acid sequence or complement sequence thereof, said difference is located two or three nucleotide bases from the liberated free 3′OH end.

14. The method of claim 12, wherein the cleavable nucleotide comprises one or more RNA bases.

15. The method of any one of claims 1 through 7 further comprising;
providing one or more blocking oligonucleotide primers comprising one or more mismatched bases at the 3′ end or one or more nucleotide analogs and a blocking group at the 3′ end, such that the 3′ end of said blocking oligonucleotide primer is unsuitable for polymerase extension when hybridized in a base-specific manner to wildtype nucleic acid sequence or bisulfite-converted unmethylated nucleic acid sequence or complement sequence thereof, wherein said blocking oligonucleotide primer comprises a portion having a nucleotide sequence that is the same as a nucleotide sequence portion in the wildtype nucleic acid sequence or bisulfite-converted unmethylated nucleic acid sequence or complement sequence thereof to which the blocking oligonucleotide primer hybridizes but has one or more nucleotide sequence mismatches to a corresponding nucleotide sequence portion in the target nucleic acid sequence or bisulfite-converted methylated nucleic acid sequence or complement sequence thereof and
blending the one or more blocking oligonucleotide primers with the sample or subsequent products prior to a polymerase extension reaction, polymerase chain reaction, or ligation reaction, whereby during hybridization said one or more blocking oligonucleotide primers preferentially hybridize in a base-specific manner to a wildtype nucleic acid sequence or bisulfite-converted unmethylated nucleic acid sequence or complement sequence thereof, thereby interfering with polymerase extension or ligation during reaction of a primer or probes hybridized in a base-specific manner to the wildtype sequence or bisulfite-converted unmethylated sequence or complement sequence thereof.

16. The method of any one of claim 3 or 6, wherein the first secondary oligonucleotide primer has a 5′ primer-specific portion and the second secondary oligonucleotide primer has a 5′ primer-specific portion, said one or more secondary oligonucleotide primer sets further comprising a third secondary oligonucleotide primer comprising the same nucleotide sequence as the 5′ primer-specific portion of the first secondary oligonucleotide primer and (d) a fourth secondary oligonucleotide primer comprising the same nucleotide sequence as the 5′ primer-specific portion of the second secondary oligonucleotide primer.

17. A method for identifying in a sample, one or more parent ribonucleic acid molecules containing a target ribonucleic acid sequence differing from ribonucleic acid sequences of other parent ribonucleic acid molecules in the sample due to alternative splicing, alternative transcript, alternative start site, alternative coding sequence, alternative non-coding sequence, exon insertion, exon deletion, intron insertion, translocation, mutation, or other rearrangement at the genome level, said method comprising:
providing a sample containing one or more parent ribonucleic acid molecules containing a target ribonucleic acid molecule potentially differing in sequence from other parent ribonucleic acid molecules;
providing one or more enzymes capable of digesting deoxyuracil (dU) containing nucleic acid molecules present in the sample;
contacting the sample with one or more enzymes capable of digesting dU containing nucleic acid molecules potentially present in the sample;
providing one or more primary oligonucleotide primer sets, each primary oligonucleotide primer set comprising (a) a first primary oligonucleotide primer that comprises a nucleotide sequence that is complementary to the RNA sequence in the parent ribonucleic acid molecule adjacent to the target ribonucleotide sequence and (b) a second primary oligonucleotide primer that comprises a nucleotide sequence that is complementary to a portion of the cDNA extension product formed from the first primary oligonucleotide primer;
blending the contacted sample, the one or more primary oligonucleotide primer sets, a deoxynucleotide mix including dUTP, a reverse transcriptase, and a DNA polymerase or a DNA polymerase with reverse-transcriptase activity to form one or more reverse-transcription/polymerase chain reaction mixtures;
subjecting the one or more reverse-transcription/polymerase chain reaction mixtures to conditions suitable for generating complementary deoxyribonucleic acid (cDNA) molecules to the target ribonucleic nucleic acid and to carry out one or more polymerase chain reaction cycles comprising a denaturation treatment, a hybridization treatment, and an extension treatment thereby forming one or more different reverse transcription/polymerase products;
providing one or more oligonucleotide probe sets, each probe set comprising (a) a first oligonucleotide probe having a 5′ primer-specific portion and a 3′ target sequence-specific portion, and (b) a second oligonucleotide probe having a 5′ target sequence-specific portion and a 3′ primer-specific portion, wherein the first and second oligonucleotide probes of a probe set are configured to hybridize, in a base specific manner, on complementary portions of a reverse transcriptase/polymerase product corresponding to the target ribonucleic acid molecule sequence;
contacting the reverse transcriptase/polymerase products with a ligase and the one or more oligonucleotide probe sets to form one or more ligation reaction mixtures;
subjecting the one or more ligation reaction mixtures to one or more ligation reaction cycles whereby the first and second probes of the one or more oligonucleotide probe sets, when hybridized to their complement, are ligated together to form ligated product sequences in the ligase reaction mixture, wherein each ligated product sequence comprises the 5′ primer-specific portion, the target-specific portions, and the 3′ primer-specific portion;
providing one or more secondary oligonucleotide primer sets, each secondary oligonucleotide primer set comprising (a) a first secondary oligonucleotide primer comprising the same nucleotide sequence as the 5′ primer-specific portion of the ligated product sequence and (b) a second secondary oligonucleotide primer comprising a nucleotide sequence that is complementary to the 3′ primer-specific portion of the ligated product sequence;
blending the ligated product sequences, the one or more secondary oligonucleotide primer sets with one or more enzymes capable of digesting deoxyuracil (dU)-containing nucleic acid molecules, a deoxynucleotide mix including dUTP, and a DNA polymerase to form one or more first polymerase chain reaction mixtures;
subjecting the one or more first polymerase chain reaction mixtures to conditions suitable for digesting deoxyuracil (dU)-containing nucleic acid molecules present in the first polymerase chain reaction mixtures and for carrying out one or more polymerase chain reaction cycles comprising a denaturation treatment, a hybridization treatment, and an extension treatment thereby forming first polymerase chain reaction products; and
detecting and distinguishing the first polymerase chain reaction products, thereby identifying the presence of one or more parent ribonucleic acid molecules containing a target ribonucleic acid sequence differing from ribonucleic acid sequences of other parent ribonucleic acid molecules in the sample due to alternative splicing, alternative transcript, alternative start site, alternative coding sequence, alternative non-coding sequence, exon insertion, exon deletion, intron insertion, translocation, mutation, or other rearrangement at the genome level.

18. A method for identifying in a sample, one or more parent ribonucleic acid molecules containing a target ribonucleic acid sequence differing from ribonucleic acid sequences of other parent ribonucleic acid molecules in the sample due to alternative splicing, alternative transcript, alternative start site, alternative coding sequence, alternative non-coding sequence, exon insertion, exon deletion, intron insertion, translocation, mutation, or other rearrangement at the genome level, said method comprising:
providing a sample containing one or more parent ribonucleic acid molecules containing a target ribonucleic acid molecule potentially differing in sequence from other parent ribonucleic acid molecules;
providing one or more enzymes capable of digesting deoxyuracil (dU)-containing nucleic acid molecules present in the sample;
contacting the sample with one or more enzymes capable of digesting dU containing nucleic acid molecules potentially present in the sample;
providing one or more primary oligonucleotide primer sets, each primary oligonucleotide primer set comprising (a) a first primary oligonucleotide primer that comprises a nucleotide sequence that is complementary to the RNA sequence in the parent ribonucleic acid molecule adjacent to the target nucleotide sequence and (b) a second primary oligonucleotide primer that comprises a nucleotide sequence that is complementary to a portion of the cDNA extension product formed from the first primary oligonucleotide primer;
blending the contacted sample, the one or more primary oligonucleotide primer sets, a deoxynucleotide mix, a reverse transcriptase and a DNA polymerase or a DNA polymerase with reverse-transcriptase activity to form one or more reverse-transcription/polymerase chain reaction mixtures;
subjecting the one or more reverse-transcription/polymerase chain reaction mixtures to conditions suitable for generating complementary deoxyribonucleic acid (cDNA) molecules to the target RNA and to carry out one or more polymerase chain reaction cycles comprising a denaturation treatment, a hybridization treatment, and an extension treatment thereby forming one or more different reverse-transcription/primary polymerase chain reaction products;
providing one or more secondary oligonucleotide primer sets, each secondary oligonucleotide primer set comprising (a) a first secondary oligonucleotide primer having a 3′ portion that is complementary to a portion of a reverse-transcription/primary polymerase chain reaction product formed from the first primary oligonucleotide primer and (b) a second secondary oligonucleotide primer having a 3′ portion that comprises a nucleotide sequence that is complementary to a portion of a reverse-transcription/primary polymerase chain reaction product formed from the first secondary oligonucleotide primer;
blending the reverse-transcription/primary polymerase chain reaction products, the one or more secondary oligonucleotide primer sets, the one or more enzymes capable of digesting deoxyuracil (dU) containing nucleic acid molecules, a deoxynucleotide mix including dUTP, and a DNA polymerase to form one or more first polymerase chain reaction mixtures;
subjecting the one or more first polymerase chain reaction mixtures to conditions suitable for digesting deoxyuracil (dU)-containing nucleic acid molecules present in the first polymerase chain reaction mixtures and for carrying out two or more polymerase chain reaction cycles comprising a denaturation treatment, a hybridization treatment, and an extension treatment thereby forming first polymerase chain reaction products; and
detecting and distinguishing the first polymerase chain reaction products, thereby identifying the presence of one or more parent ribonucleic acid molecules containing a target ribonucleic acid sequences differing from ribonucleic acid sequences of other parent ribonucleic acid molecules in the sample due to alternative splicing, alternative transcript, alternative start site, alternative coding sequence, alternative non-coding sequence, exon insertion, exon deletion, intron insertion, translocation, mutation, or other rearrangement at the genome level.

19. A method for identifying, in a sample, one or more target micro-ribonucleic acid (miRNA) molecules differing in sequence from other miRNA molecules in the sample by one or more bases, said method comprising:
providing a sample containing one or more target miRNA molecules potentially differing in sequence from other miRNA molecules in the sample by one or more bases;
providing one or more enzymes capable of digesting deoxyuracil (dU)-containing nucleic acid molecules present in the sample;
contacting the sample with one or more enzymes capable of digesting dU-containing nucleic acid molecules potentially present in the sample;
blending the contacted sample with a ligase and one or more first oligonucleotide preliminary probes comprising a 5′ phosphate, a 5′ stem-loop portion, an internal primer-specific portion within the loop region, a blocking group, and a 3′ nucleotide sequence that is complementary to a 3′ portion of the target miRNA molecule sequence to form one or more first ligation reaction mixtures;
ligating, in the one or more first ligation reaction mixtures, the one or more target miRNA molecules at their 3′ end to the 5′ phosphate of the one or more first oligonucleotide preliminary probes to generate chimeric nucleic acid molecules comprising the target miRNA molecule sequence, if present in the sample, appended to the one or more first oligonucleotide preliminary probes;
providing one or more primary oligonucleotide primer sets, each primer set comprising (a) a first primary oligonucleotide primer comprising a nucleotide sequence that is complementary to the internal primer-specific portion of the first oligonucleotide preliminary probe, and (b) a second primary oligonucleotide primer comprising a 5′ primer-specific portion and a 3′ portion, wherein the second primary oligonucleotide primer may be the same or may differ from other second primary oligonucleotide primers in other sets;
blending the one or more first ligation reaction mixtures comprising chimeric nucleic acid molecules, the one or more primary oligonucleotide primer sets, the one or more enzymes capable of digesting deoxyuracil (dU)-containing nucleic acid molecules in the sample, a deoxynucleotide mix including dUTP, and a reverse transcriptase and a DNA polymerase or a DNA polymerase with reverse-transcriptase activity to form one or more reverse-transcription/polymerase chain reaction mixtures,
subjecting the one or more reverse-transcription/polymerase chain reaction mixtures to conditions suitable for digesting deoxyuracil (dU)-containing nucleic acid molecules present in the reverse-transcription/polymerase chain reaction mixtures to conditions suitable for generating complementary deoxyribonucleic acid (cDNA) molecules to the chimeric nucleic acid molecules, and to one or more polymerase chain reaction cycles comprising a denaturation treatment, a hybridization treatment, and an extension treatment thereby forming one or more different primary reverse-transcription/polymerase chain reaction products comprising the 5′ primer-specific portion, a nucleotide sequence corresponding to the target miRNA molecule sequence, and the complement of the internal primer-specific portion, and complements thereof;
providing one or more oligonucleotide probe sets, each probe set comprising (a) a first oligonucleotide probe having a 5′ primer-specific portion and a 3′ target sequence-specific portion, and (b) a second oligonucleotide probe having a 5′ target sequence-specific portion, a portion complementary to a primary extension product, and a 3′ primer-specific portion, wherein the first and second oligonucleotide probes of a probe set are configured to hybridize, in a base specific manner, on complementary portions of a primary reverse-transcription/polymerase chain reaction product corresponding to the target miRNA molecule sequence, or complement thereof;
contacting the primary reverse-transcription/polymerase chain reaction products with a ligase and the one or more oligonucleotide probe sets to form one or more second ligation reaction mixtures;
subjecting the one or more second ligation reaction mixtures to one or more ligation reaction cycles whereby the first and second oligonucleotide probes of the one or more oligonucleotide probe sets, when hybridized to their complement, are ligated together to form ligated product sequences in the ligation reaction mixture, wherein each ligated product sequence comprises the 5′ primer-specific portion, the target-specific portions, and the 3′ primer-specific portion;
providing one or more secondary oligonucleotide primer sets, each secondary oligonucleotide primer set comprising (a) a first secondary oligonucleotide primer comprising the same nucleotide sequence as the 5′ primer-specific portion of the ligated product sequence and (b) a second secondary oligonucleotide primer comprising a nucleotide sequence that is complementary to the 3′ primer-specific portion of the ligated product sequence;
blending the ligated product sequences and the one or more secondary oligonucleotide primer sets, with one or more enzymes capable of digesting deoxyuracil (dU)-containing nucleic acid molecules, a deoxynucleotide mix including dUTP, and a DNA polymerase to form one or more second polymerase chain reaction mixtures;
subjecting the one or more second polymerase chain reaction mixtures to conditions suitable for digesting deoxyuracil (dU)-containing nucleic acid molecules present in the second polymerase chain reaction mixtures and for carrying out one or more polymerase chain reaction cycles comprising a denaturation treatment, a hybridization treatment, and an extension treatment thereby forming secondary polymerase chain reaction products; and
detecting and distinguishing the secondary polymerase chain reaction products in the one or more reactions thereby identifying one or more target miRNA molecules differing in sequence from other miRNA molecules in the sample by one or more bases.

20. A method for identifying, in a sample, one or more target micro-ribonucleic acid (miRNA) molecules differing in sequence from other miRNA molecules in the sample by one or more bases, said method comprising:
providing a sample containing one or more target miRNA molecules potentially differing in sequence from other miRNA molecules in the sample by one or more bases;
providing one or more enzymes capable of digesting deoxyuracil (dU)-containing nucleic acid molecules present in the sample;
contacting the sample with one or more enzymes capable of digesting dU-containing nucleic acid molecules potentially present in the sample;
blending the contacted sample with a ligase and one or more first oligonucleotide probes comprising a 5′ phosphate, a 5′ stem-loop portion, an internal primer-specific portion within the loop region, a blocking group, and a 3′ nucleotide sequence that is complementary to a 3′ portion of the target miRNA molecule sequence to form one or more ligation reaction mixtures;
ligating, in the one or more ligation reaction mixtures, the one or more target miRNA molecules at their 3′ end to the 5′ phosphate of the one or more first oligonucleotide probes to generate chimeric nucleic acid molecules comprising the target miRNA molecule sequence, if present in the sample, appended to the one or more first oligonucleotide probes;
providing one or more primary oligonucleotide primer sets, each primer set comprising (a) a first primary oligonucleotide primer comprising a nucleotide sequence that is complementary to the internal primer-specific portion of the first oligonucleotide probe, and (b) a second primary oligonucleotide primer comprising a 5′ primer-specific portion and a 3′ portion, wherein the second primary oligonucleotide primer may be the same or may differ from other second primary oligonucleotide primers in other sets;
blending the one or more ligation reaction mixtures comprising chimeric nucleic acid molecules, the one or more primary oligonucleotide primer sets, a deoxynucleotide mix, and a reverse transcriptase and a DNA polymerase or a DNA polymerase with reverse-transcriptase activity to form one or more reverse-transcription/polymerase chain reaction mixtures,
subjecting the one or more reverse-transcription/polymerase chain reaction mixtures to conditions suitable for generating complementary deoxyribonucleic acid (cDNA) molecules to the chimeric nucleic acid molecules, and to one or more polymerase chain reaction cycles comprising a denaturation treatment, a hybridization treatment, and an extension treatment thereby forming one or more different primary reverse-transcription/polymerase chain reaction products comprising the 5′ primer-specific portion, a nucleotide sequence corresponding to the target miRNA molecule sequence, and the complement of the internal primer-specific portion, and complements thereof;
providing one or more secondary oligonucleotide primer sets, each secondary oligonucleotide primer set comprising (a) a first secondary oligonucleotide primer having a 5′ primer-specific portion and a 3′ portion that is complementary to a portion of an extension product formed from the first primary oligonucleotide primer and (b) a second secondary oligonucleotide primer having a 5′ primer-specific portion and a 3′ portion that comprises a nucleotide sequence that is complementary to a portion of an extension product formed from the first secondary oligonucleotide primer;
blending the primary reverse-transcription/polymerase chain reaction products, the one or more secondary oligonucleotide primer sets, a deoxynucleotide mix, and a DNA polymerase to form one or more first polymerase chain reaction mixtures;
subjecting the one or more first polymerase chain reaction mixtures to conditions suitable for two or more polymerase chain reaction cycles comprising a denaturation treatment, a hybridization treatment, and an extension treatment, thereby forming first polymerase chain reaction products comprising a 5′ primer-specific portion of the first secondary oligonucleotide primer, a nucleotide sequence corresponding to the target miRNA molecule sequence or a complement thereof, and a complement of the other 5′ primer-specific portion second secondary oligonucleotide primer;
providing one or more tertiary oligonucleotide primer sets, each tertiary oligonucleotide primer set comprising (a) a first tertiary oligonucleotide primer comprising the same nucleotide sequence as the 5′ primer-specific portion of the first polymerase chain reaction products or their complements and (b) a second tertiary oligonucleotide primer comprising a nucleotide sequence that is complementary to the 3′ primer-specific portion of the first polymerase chain reaction products or their complements;
blending the first polymerase chain reaction process products, the one or more tertiary oligonucleotide primer sets, the one or more enzymes capable of digesting deoxyuracil (dU)-containing nucleic acid molecules, a deoxynucleotide mix including dUTP, and a DNA polymerase to form one or more second polymerase chain reaction mixtures;
subjecting the one or more second polymerase chain reaction mixtures to conditions suitable for digesting deoxyuracil (dU) containing nucleic acid molecules present in the second polymerase chain reaction mixtures and for carrying out one or more polymerase chain reaction cycles comprising a denaturation treatment, a hybridization treatment, and an extension treatment thereby forming second polymerase chain reaction products; and
detecting and distinguishing the second polymerase chain reaction products, thereby identifying one or more target miRNA molecules differing in sequence from other miRNA molecules in the sample by one or more bases.

21. A method for identifying, in a sample, one or more target micro-ribonucleic acid (miRNA) molecules differing in sequence from other miRNA molecules in the sample by one or more bases, said method comprising:
providing a sample containing one or more target miRNA molecules potentially differing in sequence from other miRNA molecules in the sample by one or more bases;
providing one or more enzymes capable of digesting deoxyuracil (dU)-containing nucleic acid molecules present in the sample;
contacting the sample with one or more enzymes capable of digesting dU-containing nucleic acid molecules potentially present in the sample;
blending the contacted sample with ATP and a Poly(A) polymerase to form a Poly(A) polymerase reaction mixture;
subjecting the Poly(A) polymerase reaction mixture to conditions suitable for appending homopolymer A to the 3′ ends of the one or more target miRNA molecules potentially present in the sample;
providing one or more primary oligonucleotide primer sets, each primer set comprising (a) a first primary oligonucleotide primer comprising a 5′ primer-specific portion, an internal poly dT portion, and a 3′ portion comprising from 1 to 10 bases complementary to the 3′ end of the target miRNA, wherein the first primary oligonucleotide primer may be the same or may differ from other first primary oligonucleotide primers in other sets, and (b) a second primary oligonucleotide primer comprising a 5′ primer-specific portion and a 3′ portion, wherein the second primary oligonucleotide primer may be the same or may differ from other second primary oligonucleotide primers in other sets;
blending the Poly(A) polymerase reaction mixture, the one or more primary oligonucleotide primer sets, the one or more enzymes capable of digesting deoxyuracil (dU)-containing nucleic acid molecules in the sample, a deoxynucleotide mix including dUTP, and a reverse transcriptase and a DNA polymerase or a DNA polymerase with reverse-transcriptase activity to form one or more reverse-transcription/polymerase chain reaction mixtures;
subjecting the one or more reverse-transcription/polymerase chain reaction mixtures to conditions suitable for digesting deoxyuracil (dU)-containing nucleic acid molecules present in the reverse-transcription/polymerase chain reaction mixtures, then to conditions suitable for generating complementary deoxyribonucleic acid (cDNA) molecules to the target miRNA sequences with 3′ polyA tails, and to one or more polymerase chain reaction cycles comprising a denaturation treatment, a hybridization treatment, and an extension treatment thereby forming one or more different reverse-transcription/polymerase chain reaction products comprising the 5′ primer-specific portion of the second primary oligonucleotide primer, a nucleotide sequence corresponding to the target miRNA molecule sequence, a poly dA region, and the complement of the 5′ primer-specific portion of the first primary oligonucleotide primer, and complements thereof;
providing one or more oligonucleotide probe sets, each probe set comprising (a) a first oligonucleotide probe having a 5′ primer-specific portion and a 3′ target sequence-specific portion, and (b) a second oligonucleotide probe having a 5′ target sequence-specific portion, a portion complementary to the one or more reverse-transcription/polymerase chain reaction products, and a 3′ primer-specific portion, wherein the first and second oligonucleotide probes of a probe set are configured to hybridize, in a base specific manner, to complementary portions of the one or more reverse-transcription/polymerase chain reaction products corresponding to the target miRNA molecule sequence, or complement thereof;
contacting the one or more reverse-transcription/polymerase chain reaction products with a ligase and the one or more oligonucleotide probe sets to form one or more ligation reaction mixtures;
subjecting the one or more ligation reaction mixtures to one or more ligation reaction cycles whereby the first and second oligonucleotide probes of the one or more oligonucleotide probe sets, when hybridized to their complement, are ligated together to form ligated product sequences in the ligation reaction mixture, wherein each ligated product sequence comprises the 5′ primer-specific portion, the target-specific portions, and the 3′ primer-specific portion;
providing one or more secondary oligonucleotide primer sets, each secondary oligonucleotide primer set comprising (a) a first secondary oligonucleotide primer comprising the same nucleotide sequence as the 5′ primer-specific portion of the ligated product sequence and (b) a second secondary oligonucleotide primer comprising a nucleotide sequence that is complementary to the 3′ primer-specific portion of the ligated product sequence;
blending the ligated product sequences and the one or more secondary oligonucleotide primer sets, with one or more enzymes capable of digesting deoxyuracil (dU)-containing nucleic acid molecules, a deoxynucleotide mix including dUTP, and a DNA polymerase to form one or more first polymerase chain reaction mixtures;
subjecting the one or more first polymerase chain reaction mixtures to conditions suitable for digesting deoxyuracil (dU)-containing nucleic acid molecules present in the first polymerase chain reaction mixtures and for carrying out one or more polymerase chain reaction cycles comprising a denaturation treatment, a hybridization treatment, and an extension treatment thereby forming secondary polymerase chain reaction products; and
detecting and distinguishing the secondary polymerase chain reaction products, thereby identifying one or more target miRNA molecules differing in sequence from other miRNA molecules in the sample by one or more bases.

22. A method for identifying, in a sample, one or more target micro-ribonucleic acid (miRNA) molecules differing in sequence from other miRNA molecules in the sample by one or more bases, said method comprising:
providing a sample containing one or more target miRNA molecules potentially differing in sequence from other miRNA molecules in the sample by one or more bases;
providing one or more enzymes capable of digesting deoxyuracil (dU)-containing nucleic acid molecules present in the sample;
contacting the sample with one or more enzymes capable of digesting dU-containing nucleic acid molecules potentially present in the sample;
blending the contacted sample with ATP and a Poly(A) polymerase to form a Poly(A) polymerase reaction mixture;
subjecting the Poly(A) polymerase reaction mixture to conditions suitable for appending a homopolymer A to the 3′ ends of the one or more target miRNA molecules potentially present in the sample;
providing one or more primary oligonucleotide primer sets, each primer set comprising (a) a first primary oligonucleotide primer comprising a 5′ primer-specific portion, an internal poly dT portion, and a 3′ portion comprising from 1 to 10 bases complementary to the 3′ end of the target miRNA, wherein the first primary oligonucleotide primer may be the same or may differ from other first primary oligonucleotide primers in other sets, and (b) a second primary oligonucleotide primer comprising a 5′ primer-specific portion and a 3′ portion, wherein the second primary oligonucleotide primer may be the same or may differ from other second primary oligonucleotide primers in other sets;
blending the Poly(A) polymerase reaction mixture potentially comprising target miRNA sequences with 3′ polyA tails, the one or more primary oligonucleotide primer sets, a deoxynucleotide mix, and a reverse transcriptase and a DNA polymerase or a DNA polymerase with reverse-transcriptase activity to form one or more reverse-transcription/polymerase chain reaction mixtures;
subjecting the one or more reverse-transcription/polymerase chain reaction mixtures to conditions suitable for generating complementary deoxyribonucleic acid (cDNA) molecules to the target miRNA sequences with 3′ polyA tails, and to one or more polymerase chain reaction cycles comprising a denaturation treatment, a hybridization treatment, and an extension treatment, thereby forming one or more different reverse-transcription/polymerase chain reaction products comprising the 5′ primer-specific portion of the second primary oligonucleotide primer, a nucleotide sequence corresponding to the target miRNA molecule sequence, a poly dA region, and the complement of the 5′ primer-specific portion of the first primary oligonucleotide primer, and complements thereof;
providing one or more secondary oligonucleotide primer sets, each secondary oligonucleotide primer set comprising (a) a first secondary oligonucleotide primer having a 5′ primer-specific portion and a 3′ portion that is complementary to a portion of a reverse-transcription/polymerase chain reaction product formed from the first primary oligonucleotide primer and (b) a second secondary oligonucleotide primer having a 5′ primer-specific portion and a 3′ portion that comprises a nucleotide sequence that is complementary to a portion of a reverse-transcription/polymerase chain reaction product formed from the first secondary oligonucleotide primer;
blending the reverse-transcription/polymerase chain reaction products, the one or more secondary oligonucleotide primer sets, a deoxynucleotide mix, and a DNA polymerase to form one or more first polymerase chain reaction mixtures;
subjecting the one or more first polymerase chain reaction mixtures to conditions suitable for two or more polymerase chain reaction cycles comprising a denaturation treatment, a hybridization treatment, and an extension treatment, thereby forming first polymerase chain reaction products comprising a 5′ primer-specific portion, a nucleotide sequence corresponding to the target miRNA molecule sequence or a complement thereof; and a complement of the other 5′ primer-specific portion;
providing one or more tertiary oligonucleotide primer sets, each tertiary oligonucleotide primer set comprising (a) a first tertiary oligonucleotide primer comprising the same nucleotide sequence as the 5′ primer-specific portion of the first polymerase chain reaction product sequence and (b) a second tertiary oligonucleotide primer comprising a nucleotide sequence that is complementary to the 3′ primer-specific portion of the first polymerase chain reaction product sequence;
blending the first polymerase chain reaction products, the one or more tertiary oligonucleotide primer sets, the one or more enzymes capable of digesting deoxyuracil (dU) containing nucleic acid molecules, a deoxynucleotide mix including dUTP, and a DNA polymerase to form one or more second polymerase chain reaction mixtures;
subjecting the one or more second polymerase chain reaction mixtures to conditions suitable for digesting deoxyuracil (dU)-containing nucleic acid molecules present in the first polymerase chain reaction mixtures, and one or more polymerase chain reaction cycles comprising a denaturation treatment, a hybridization treatment, and an extension treatment thereby forming second polymerase chain reaction products; and
detecting and distinguishing the second polymerase chain reaction products in the one or more reactions thereby identifying one or more target miRNA molecules differing in sequence from other miRNA molecules in the sample by one or more bases.

23. The method of any one of claims 19 through 22 wherein the 3′ portion of the second primary oligonucleotide primer comprises ribo-G and/or G nucleotide analogue, wherein the reverse transcriptase appends two or three cytosine nucleotides to the 3′ end of the complementary deoxyribonucleic acid products of the target miRNAs, enabling transient hybridization to the 3′ end of the second primary oligonucleotide primer, enabling the reverse transcriptase to undergo strand switching and to extend the complementary deoxyribonucleic acid products to include the complementary sequence of the 5′ primer-specific portion of the second primary oligonucleotide primer to form the one or more different first polymerase chain reaction products comprising a 5′ primer-specific portion, a nucleotide sequence portion corresponding to the target miRNA molecule sequence or a complement thereof, a further portion, and a complement of the other 5′ primer-specific portion.

24. The method of any one of claims 19 through 22 wherein the 3′ portion of the second primary oligonucleotide primers contains from 6 to 14 bases comprising, from 5′ to 3′, three ribo-G or G bases, followed by additional bases that are the same as the 5′ end of the target miRNA sequences, wherein the reverse transcriptase appends two or three cytosine residues to the 3′ end of the initial complementary deoxyribonucleic acid extension products of the target miRNAs, and wherein subsequent to when the denaturation treatment of the polymerase chain reaction is initiated the conditions are adjusted to enable transient hybridization to the 3′ end of the second primary oligonucleotide primers to the 3′ end of the complementary deoxyribonucleic acid extension products, allowing for extension of either one or both the second primary oligonucleotide primers and the complementary deoxyribonucleic acid extension products to form the one or more different primary reverse-transcription/polymerase chain reaction products comprising a 5′ primer-specific portion, a nucleotide sequence portion corresponding to the target miRNA molecule sequence or a complement thereof, a further portion, and a complement of the other 5′ primer-specific portion.

25. The method of any one of claim 1, 4, or 17, wherein the second oligonucleotide probe of the oligonucleotide probe set further comprises a unitaq detection portion, thereby forming ligated product sequences comprising the 5′ primer-specific portion, the target-specific portions, the unitaq detection portion, and the 3′ primer-specific portion, said method further comprising:
providing one or more unitaq detection probes, wherein each unitaq detection probe hybridizes to a complementary unitaq detection portion and said detection probe comprises a quencher molecule and a detectable label separated from the quencher molecule;
adding the one or more unitaq detection probes to the second polymerase chain reaction mixture; and
hybridizing the one or more unitaq detection probes to complementary unitaq detection portions on the ligated product sequence or complement thereof during said subjecting the second polymerase chain reaction mixture to conditions suitable for one or more polymerase chain reaction cycles, wherein the quencher molecule and the detectable label are cleaved from the one or more unitaq detection probes during the extension treatment and said detecting involves the detection of the cleaved detectable label.

26. The method of any one of claim 2, 3, 5, 6, 7, or 18, wherein one primary oligonucleotide primer or one secondary oligonucleotide primer further comprises a unitaq detection portion, thereby forming extension product sequences comprising the 5′ primer-specific portion, the target-specific portions, the unitaq detection portion, and the complement of the other 5′ primer-specific portion, and complements thereof, said method further comprising:
providing one or more unitaq detection probes, wherein each unitaq detection probe hybridizes to a complementary unitaq detection portion and said detection probe comprises a quencher molecule and a detectable label separated from the quencher molecule;
adding the one or more unitaq detection probes to the one or more first or second polymerase chain reaction mixtures; and
hybridizing the one or more unitaq detection probes to complementary unitaq detection portions on the ligated product sequence or complement thereof during polymerase chain reaction cycles after the first polymerization chain reaction, wherein the quencher molecule and the detectable label are cleaved from the one or more unitaq detection probes during the extension treatment and said detecting involves the detection of the cleaved detectable label.

27. The method of any one of claim 1, 4, 17, 19, or 21, wherein one or both oligonucleotide probes of the oligonucleotide probe set comprises a portion that has no or one nucleotide sequence mismatch when hybridized in a base-specific manner to the target nucleic acid sequence or bisulfite-converted methylated nucleic acid sequence or complement sequence thereof, but have one or more additional nucleotide sequence mismatches that interferes with ligation when said oligonucleotide probe hybridizes in a base-specific manner to a corresponding nucleotide sequence portion in the wildtype nucleic acid sequence or bisulfite-converted unmethylated nucleic acid sequence or complement sequence thereof.

28. The method of any one of claim 1, 4, or 17, wherein the 3′ portion of the first oligonucleotide probe of the oligonucleotide probe set comprises a cleavable nucleotide or nucleotide analogue and a blocking group, such that the 3′ end is unsuitable for polymerase extension or ligation, said method further comprising;
cleaving the cleavable nucleotide or nucleotide analog of the first oligonucleotide probe when said probe is hybridized to it complementary target nucleotide sequence of the primary extension product, thereby liberating a 3′OH on the first oligonucleotide probe prior to said ligating.

29. The method of claim 28, wherein the one or more first oligonucleotide probe of the oligonucleotide probe set comprises a sequence that differs from the target nucleic acid sequence or bisulfite-converted methylated nucleic acid sequence or complement sequence thereof, said difference is located two or three nucleotide bases from the liberated free 3′OH end.

30. The method of any one of claim 1, 4, or 17, wherein the second oligonucleotide probe has, at its 5′ end, an overlapping identical nucleotide with the 3′ end of the first oligonucleotide probe, and, upon hybridization of the first and second oligonucleotide probes of a probe set at adjacent positions on a complementary target nucleotide sequence of a primary extension product to form a junction, the overlapping identical nucleotide of the second oligonucleotide probe forms a flap at the junction with the first oligonucleotide probe, said method further comprising:
cleaving the overlapping identical nucleotide of the second oligonucleotide probe with an enzyme having 5′ nuclease activity thereby liberating a phosphate at the 5′ end of the second oligonucleotide probe prior to said ligating.

31. The method of any one of claim 1, 4, or 17, wherein the one or more oligonucleotide probe sets further comprise a third oligonucleotide probe having a target-specific portion, wherein the second and third oligonucleotide probes of a probe set are configured to hybridize adjacent to one another on the target nucleotide sequence with a junction between them to allow ligation between the second and third oligonucleotide probes to form a ligated product sequence comprising the first, second, and third oligonucleotide probes of a probe set.

32. The method of any one of claims 1 through 31, wherein the sample is selected from the group consisting of tissue, cells, serum, blood, plasma, amniotic fluid, sputum, urine, bodily fluids, bodily secretions, bodily excretions, cell-free circulating nucleic acids, cell-free circulating tumor nucleic acids, cell-free circulating fetal nucleic acids in pregnant woman, circulating tumor cells, tumor, tumor biopsy, and exosomes.

33. The method of any one of claims 1 through 31, wherein the one or more target nucleotide sequences are low-abundance nucleic acid molecules comprising one or more nucleotide base mutations, insertions, deletions, translocations, splice variants, miRNA variants, alternative transcripts, alternative start sites, alternative coding sequences, alternative non-coding sequences, alternative splicings, exon insertions, exon deletions, intron insertions, or other rearrangement at the genome level and/or methylated nucleotide bases.

34. The method of claim 33, wherein the low-abundance nucleic acid molecules with one or more nucleotide base mutations, insertions, deletions, translocations, splice variants, miRNA variants, alternative transcripts, alternative start sites, alternative coding sequences, alternative non-coding sequences, alternative splicings, exon insertions, exon deletions, intron insertions, or other rearrangement at the genome level, and/or methylated nucleotide bases are identified and distinguished from a high-abundance of nucleic acid molecules in the sample having a similar nucleotide sequence as the low abundance nucleic acid molecules but without the one or more nucleotide base mutations, insertions, deletions, translocations, splice variants, miRNA variants, alternative transcripts, alternative start sites, alternative coding sequences, alternative non-coding sequences, alternative splicings, exon insertions, exon deletions, intron insertions, or other rearrangement at the genome level, and/or methylated nucleotide bases.

35. The method of claim 34, wherein the copy number of one or more low-abundance target nucleotide sequences are quantified relative to the copy number of the high-abundance nucleic acid molecules in the sample.

36. The method of any one of claims 1 through 31, wherein the one or more target nucleotide sequences are quantified or enumerated.

37. The method of claim 36, wherein the one or more target nucleotide sequences are quantified or enumerated relative to other nucleotide sequences in the sample or other samples undergoing the identical subsequent steps.

38. The method of claim 37, wherein the relative copy number of one or more target nucleotide sequences are quantified or enumerated.

39. The method of any one of claims 1 through 31 further comprising:
diagnosing or prognosing a disease state based on said identifying.

40. The method of any one of claims 1 through 31 further comprising:
distinguishing a genotype or disease predisposition based on said identifying.

41. A method of diagnosing or prognosing a disease state of cells or tissue based on identifying the presence or level of a plurality of disease-specific and/or cell/tissue-specific DNA, RNA, and/or protein markers in a biological sample of an individual, wherein the plurality of markers is in a set comprising from 6-12 markers, 12-24 markers, 24-36 markers, 36-48 markers, 48-72 markers, 72-96 markers, or >96 markers, wherein each marker in a given set is selected by having any one or more of the following criteria:
present, or above a cutoff level, in >50% of biological samples of the disease cells or tissue from individuals diagnosed with the disease state;
absent, or below a cutoff level, in >95% of biological samples of the normal cells or tissue from individuals without the disease state;
present, or above a cutoff level, in >50% of biological samples comprising cells, serum, blood, plasma, amniotic fluid, sputum, urine, bodily fluids, bodily secretions, bodily excretions, or fractions thereof, from individuals diagnosed with the disease state;
absent, or below a cutoff level, in >95% of biological samples comprising cells, serum, blood, plasma, amniotic fluid, sputum, urine, bodily fluids, bodily secretions, bodily excretions, or fractions thereof, from individuals without the disease state;
present with a z-value of >1.65 in the biological sample comprising cells, serum, blood, plasma, amniotic fluid, sputum, urine, bodily fluids, bodily secretions, bodily excretions, or fractions thereof, from individuals diagnosed with the disease state;
and, wherein at least 50% of the markers in a set each comprise one or more methylated residues, and/or wherein at least 50% of the markers in a set that are present, or above a cutoff level, or present with a z-value of >1.65, comprise of one or more methylated residues, in the biological sample comprising cells, serum, blood, plasma, amniotic fluid, sputum, urine, bodily fluids, bodily secretions, bodily excretions, or fractions thereof, from at least 50% of individuals diagnosed with the disease state, said method comprising:
obtaining a biological sample including cell-free DNA, RNA, and/or protein originating from the cells or tissue and from one or more other tissues or cells, wherein the biological sample is selected from the group consisting of cells, serum, blood, plasma, amniotic fluid, sputum, urine, bodily fluids, bodily secretions, and bodily excretions, or fractions thereof;
fractionating the sample into one or more fractions, wherein at least one fraction comprises exosomes, tumor-associated vesicles, other protected states, or cell-free DNA, RNA, and/or protein;
subjecting nucleic acid molecules in the one or more fractions to a bisulfite treatment under conditions suitable to convert unmethylated cytosine residues to uracil residues;
carrying out at least two enrichment steps for 50% or more disease-specific and/or cell/tissue-specific DNA, RNA, and/or protein markers during either said fractionating and/or by carrying out a nucleic acid amplification step; and
performing one or more assays to detect and distinguish the plurality of disease-specific and/or cell/tissue-specific DNA, RNA, and/or protein markers, thereby identifying their presence or levels in the sample, wherein individuals are diagnosed or prognosed with the disease state if a minimum of 2 or 3 markers are present or are above a cutoff level in a marker set comprising from 6-12 markers; or a minimum of 3, 4, or 5 markers are present or are above a cutoff level in a marker set comprising from 12-24 markers; or a minimum of 3, 4, 5, or 6 markers are present or are above a cutoff level in a marker set comprising from 24-36 markers; or a minimum of 4, 5, 6, 7, or 8 markers are present or are above a cutoff level in a marker set comprising from 36-48 markers; or a minimum of 6, 7, 8, 9, 10, 11, or 12 markers are present or are above a cutoff level in a marker set comprising from 48-72 markers, or a minimum of 7, 8, 9, 10, 11, 12 or 13 markers are present or are above a cutoff level in a marker set comprising from 72-96 markers, or a minimum of 8, 9, 10, 11, 12, 13 or “n”/12 markers are present or are above a cutoff level in a marker set comprising 96−“n” markers, when “n”>168 markers.

42. A method of diagnosing or prognosing a disease state of a solid tissue cancer including colorectal adenocarcinoma, stomach adenocarcinoma, esophageal carcinoma, breast lobular and ductal carcinoma, uterine corpus endometrial carcinoma, ovarian serous cystadenocarcinoma, cervical squamous cell carcinoma and adenocarcinoma, uterine carcinosarcoma, lung adenocarcinoma, lung squamous cell carcinoma, head & neck squamous cell carcinoma, prostate adenocarcinoma, invasive urothelial bladder cancer, liver hepatoceullular carcinoma, pancreatic ductal adenocarcinoma, or gallbladder adenocarcinoma, based on identifying the presence or level of a plurality of disease-specific and/or cell/tissue-specific DNA, RNA, and/or protein markers in a biological sample of an individual, wherein the plurality of markers is in a set comprising from 48-72 total cancer markers, 72-96 total cancer markers or ≥96 total cancer markers, wherein on average greater than one quarter such markers in a given set cover each of the aforementioned major cancers being tested, wherein each marker in a given set for a given solid tissue cancer is selected by having any one or more of the following criteria for that solid tissue cancer:
present, or above a cutoff level, in >50% of biological samples of a given cancer tissue from individuals diagnosed with a given solid tissue cancer;
absent, or below a cutoff level, in >95% of biological samples of the normal tissue from individuals without that given solid tissue cancer;
present, or above a cutoff level, in >50% of biological samples comprising cells, serum, blood, plasma, amniotic fluid, sputum, urine, bodily fluids, bodily secretions, bodily excretions, or fractions thereof, from individuals diagnosed with a given solid tissue cancer;
absent, or below a cutoff level, in >95% of biological samples comprising cells, serum, blood, plasma, amniotic fluid, sputum, urine, bodily fluids, bodily secretions, bodily excretions, or fractions thereof, from individuals without that given solid tissue cancer;
present with a z-value of >1.65 in the biological sample comprising cells, serum, blood, plasma, amniotic fluid, sputum, urine, bodily fluids, bodily secretions, bodily excretions, or fractions thereof, from individuals diagnosed with a given solid tissue cancer;
and, wherein at least 50% of the markers in a set each comprise one or more methylated residues, and/or wherein at least 50% of the markers in a set that are present, or above a cutoff level, or present with a z-value of >1.65, comprise of one or more methylated residues, in the biological sample comprising cells, serum, blood, plasma, amniotic fluid, sputum, urine, bodily fluids, bodily secretions, bodily excretions, or fractions thereof, from at least 50% of individuals diagnosed with a given solid tissue cancer, said method comprising:
obtaining a biological sample including cell-free DNA, RNA, and/or protein originating from the cells or tissue and from one or more other tissues or cells, wherein the biological sample is selected from the group consisting of cells, serum, blood, plasma, amniotic fluid, sputum, urine, bodily fluids, bodily secretions, and bodily excretions, or fractions thereof;
fractionating the sample into one or more fractions, wherein at least one fraction comprises exosomes, tumor-associated vesicles, other protected states, or cell-free DNA, RNA, and/or protein;
subjecting the nucleic acid molecules in the one or more fractions to a bisulfite treatment under conditions suitable to convert unmethylated cytosine residues to uracil residues;
carrying out at least two enrichment steps for 50% or more of the given solid tissue cancer-specific and/or cell/tissue-specific DNA, RNA, and/or protein markers during either said fractionating and/or by carrying out a nucleic acid amplification step; and
performing one or more assays to detect and distinguish the plurality of cancer-specific and/or cell/tissue-specific DNA, RNA, and/or protein markers, thereby identifying their presence or levels in the sample, wherein individuals are diagnosed or prognosed with the a solid-tissue cancer if a minimum of 4 markers are present or are above a cutoff level in a marker set comprising from 48-72 total cancer markers; or a minimum of 5 markers are present or are above a cutoff level in a marker set comprising from 72-96 total cancer markers; or a minimum of 6 or “n”/18 markers are present or are above a cutoff level in a marker set comprising 96 to “n” total cancer markers, when “n”>96 total cancer markers.

43. The method of claim 42, wherein each marker in a given set for a given solid tissue cancer is selected by having any one or more of the following criteria for that solid tissue cancer:
present, or above a cutoff level, in >66% of biological samples of a given cancer tissue from individuals diagnosed with a given solid tissue cancer;
absent, or below a cutoff level, in >95% of biological samples of the normal tissue from individuals without that given solid tissue cancer;
present, or above a cutoff level, in >66% of biological samples comprising cells, serum, blood, plasma, amniotic fluid, sputum, urine, bodily fluids, bodily secretions, bodily excretions, or fractions thereof, from individuals diagnosed with a given solid tissue cancer;
absent, or below a cutoff level, in >95% of biological samples comprising cells, serum, blood, plasma, amniotic fluid, sputum, urine, bodily fluids, bodily secretions, bodily excretions, or fractions thereof, from individuals without that given solid tissue cancer;
present with a z-value of >1.65 in the biological sample comprising cells, serum, blood, plasma, amniotic fluid, sputum, urine, bodily fluids, bodily secretions, bodily excretions, or fractions thereof, from individuals diagnosed with a given solid tissue cancer.

44. A method of diagnosing or prognosing a disease state of and identifying the most likely specific tissue(s) of origin of a solid tissue cancer in the following groups: Group 1 (colorectal adenocarcinoma, stomach adenocarcinoma, esophageal carcinoma); Group 2 (breast lobular and ductal carcinoma, uterine corpus endometrial carcinoma, ovarian serous cystadenocarcinoma, cervical squamous cell carcinoma and adenocarcinoma, uterine carcinosarcoma); Group 3 (lung adenocarcinoma, lung squamous cell carcinoma, head & neck squamous cell carcinoma); Group 4 (prostate adenocarcinoma, invasive urothelial bladder cancer); and/or Group 5 (liver hepatoceullular carcinoma, pancreatic ductal adenocarcinoma, or gallbladder adenocarcinoma) based on identifying the presence or level of a plurality of disease-specific and/or cell/tissue-specific DNA, RNA, and/or protein markers in a biological sample of an individual, wherein the plurality of markers is in a set comprising from 36-48 group-specific cancer markers, 48-64 group-specific cancer markers, or 64 group-specific cancer markers, wherein on average greater than one third of such markers in a given set cover each of the aforementioned cancers being tested within that group, wherein each marker in a given set for a given solid tissue cancer is selected by having any one or more of the following criteria for that solid tissue cancer:
present, or above a cutoff level, in >50% of biological samples of a given cancer tissue from individuals diagnosed with a given solid tissue cancer;
absent, or below a cutoff level, in >95% of biological samples of the normal tissue from individuals without that given solid tissue cancer;
present, or above a cutoff level, in >50% of biological samples comprising cells, serum, blood, plasma, amniotic fluid, sputum, urine, bodily fluids, bodily secretions, bodily excretions, or fractions thereof, from individuals diagnosed with a given solid tissue cancer;
absent, or below a cutoff level, in >95% of biological samples comprising cells, serum, blood, plasma, amniotic fluid, sputum, urine, bodily fluids, bodily secretions, bodily excretions, or fractions thereof, from individuals without that given solid tissue cancer;
present with a z-value of >1.65 in the biological sample comprising cells, serum, blood, plasma, amniotic fluid, sputum, urine, bodily fluids, bodily secretions, bodily excretions, or fractions thereof, from individuals diagnosed with a given solid tissue cancer;
and, wherein at least 50% of the markers in a set each comprise one or more methylated residues, and/or wherein at least 50% of the markers in a set that are present, or above a cutoff level, or present with a z-value of >1.65 comprise one or more methylated residues, in the biological sample comprising cells, serum, blood, plasma, amniotic fluid, sputum, urine, bodily fluids, bodily secretions, bodily excretions, or fractions thereof, from at least 50% of individuals diagnosed with a given solid tissue cancer, said method comprising:
obtaining the biological sample, the biological sample including cell-free DNA, RNA, and/or protein originating from the cells or tissue and from one or more other tissues or cells, wherein the biological sample is selected from the group consisting of cells, serum, blood, plasma, amniotic fluid, sputum, urine, bodily fluids, bodily secretions, bodily excretions, or fractions thereof;
fractionating the sample into one or more fractions, wherein at least one fraction comprises exosomes, tumor-associated vesicles, other protected states, or cell-free DNA, RNA, and/or protein;
subjecting the nucleic acid molecules in the one or more fractions to a bisulfite treatment under conditions suitable to convert unmethylated cytosine residues to uracil residues;
carrying out at least two enrichment steps for 50% or more of the given solid tissue cancer-specific and/or cell/tissue-specific DNA, RNA, and/or protein markers during either said fractionating and/or by carrying out a nucleic acid amplification step; and
performing one or more assays to detect and distinguish the plurality of cancer-specific and/or cell/tissue-specific DNA, RNA, and/or protein markers, thereby identifying their presence or levels in the sample, wherein individuals are diagnosed or prognosed with a solid-tissue cancer if a minimum of 4 markers are present or are above a cutoff level in a marker set comprising from 36-48 group-specific cancer markers; or a minimum of 5 markers are present or are above a cutoff level in a marker set comprising from 48-64 group-specific cancer markers; or
a minimum of 6 or “n”/12 markers are present or are above a cutoff level in a marker set comprising 64 to “n” total cancer markers, when “n”>64 group-specific cancer markers.

45. The method of claim 44, wherein each marker in a given set for a given solid tissue cancer is selected by having any one or more of the following criteria for that solid tissue cancer:
present, or above a cutoff level, in >66% of biological samples of a given cancer tissue from individuals diagnosed with a given solid tissue cancer;
absent, or below a cutoff level, in >95% of biological samples of the normal tissue from individuals without that given solid tissue cancer;
present, or above a cutoff level, in >66% of biological samples comprising cells, serum, blood, plasma, amniotic fluid, sputum, urine, bodily fluids, bodily secretions, bodily excretions, or fractions thereof, from individuals diagnosed with a given solid tissue cancer;
absent, or below a cutoff level, in >95% of biological samples comprising cells, serum, blood, plasma, amniotic fluid, sputum, urine, bodily fluids, bodily secretions, bodily excretions, or fractions thereof, from individuals without that given solid tissue cancer;
present with a z-value of >1.65 in the biological sample comprising cells, serum, blood, plasma, amniotic fluid, sputum, urine, bodily fluids, bodily secretions, bodily excretions, or fractions thereof, from individuals diagnosed with a given solid tissue cancer.

46. The method of any one of claims 41 through 45, wherein the at least two enrichment steps comprise of two or more of the following steps:
capturing or separating exosomes or extracellular vesicles or markers in other protected states; capturing or separating a platelet fraction; capturing or separating circulating tumor cells; capturing or separating RNA-containing complexes; capturing or separating cfDNA-nucleosome or differentially modified cfDNA-histone complexes; capturing or separating protein targets or protein target complexes; capturing or separating auto-antibodies; capturing or separating cytokines; capturing or separating methylated cfDNA; capturing or separating marker specific DNA, cDNA, miRNA, lncRNA, ncRNA, or mRNA, or amplified complements, by hybridization to complementary capture probes in solution, on magnetic beads, or on a microarray; amplifying miRNA markers, non-coding RNA markers (lncRNA & ncRNA markers), mRNA markers, exon markers, splice-variant markers, translocation markers, or copy number variation markers in a linear or exponential manner via a polymerase extension reaction, polymerase chain reaction, bisulfite-methyl-specific polymerase chain reaction, reverse-transcription reaction, bisulfite-methyl-specific ligation reaction, and/or ligation reaction, using DNA polymerase, reverse transcriptase, DNA ligase, RNA ligase, DNA repair enzyme, RNase, RNaseH2, endonuclease, restriction endonuclease, exonuclease, CRISPR, DNA glycosylase or combinations thereof; selectively amplifying one or more target regions containing mutation markers or bisulfite-converted DNA methylation markers, while suppressing amplification of the target regions containing wild-type sequence or bisulfite-converted unmethylated sequence or complement sequence thereof, in a linear or exponential manner via a polymerase extension reaction, polymerase chain reaction, bisulfite-methyl-specific polymerase chain reaction, reverse-transcription reaction, bisulfite-methyl-specific ligation reaction, and/or ligation reaction, using DNA polymerase, reverse transcriptase, DNA ligase, RNA ligase, DNA repair enzyme, RNase, RNaseH2, endonuclease, restriction endonuclease, exonuclease, CRISPR, DNA glycosylase or combinations thereof; preferentially extending, ligating, or amplifying one or more primers or probes whose 3′-OH end has been liberated in an enzyme and sequence-dependent process; using one or more blocking oligonucleotide primers comprising one or more mismatched bases at the 3′ end or comprising one or more nucleotide analogs and a blocking group at the 3′ end under conditions that interfere with polymerase extension or ligation during said reaction of target-specific primer or probes hybridized in a base-specific manner to wildtype sequence or bisulfite-converted unmethylated sequence or complement sequence thereof.

47. The method of any one of claims 41 through 46, wherein the one or more assays to detect and distinguish the plurality of disease-specific and/or cell/tissue-specific DNA, RNA, or protein markers comprise one or more of the following:
a quantitative real-time PCR method (qPCR); a reverse transcriptase-polymerase chain reaction (RTPCR) method; a bisulfite qPCR method; a digital PCR method (dPCR); a bisulfite dPCR method; a ligation detection method, a ligase chain reaction, a restriction endonuclease cleavage method; a DNA or RNA nuclease cleavage method; a micro-array hybridization method; a peptide-array binding method; an antibody-array method; a mass spectrometry method; a liquid chromatography-tandem mass spectrometry (LC-MS/MS) method; a capillary or gel electrophoresis method; a chemiluminescence method; a fluorescence method; a DNA sequencing method; a bisulfite conversion-DNA sequencing method; an RNA sequencing method; a proximity ligation method; a proximity PCR method; a method comprising immobilizing an antibody-target complex; a method comprising immobilizing an aptamer-target complex; an immunoassay method; a method comprising a Western blot assay; a method comprising an enzyme linked immunosorbent assay (ELISA); a method comprising a high-throughput microarray-based enzyme-linked immunosorbent assay (ELISA); or a method comprising a high-throughput flow-cytometry-based enzyme-linked immunosorbent assay (ELISA).

48. The method of any one of claims 41 through 47, wherein the one or more cutoff levels of the one or more assays to detect and distinguish the plurality of disease-specific and/or cell/tissue-specific DNA, RNA, or protein markers comprise one or more of the following calculations, comparisons, or determinations, in the one or more marker assays comparing samples from the disease vs. normal individual:
marker ΔCt value is >2; marker ΔCt value is >4; ratio of detected marker-specific signal is >1.5; ratio of detected marker-specific signal is >3; ratio of marker concentrations is >1.5; ratio of marker concentrations is >3; enumerated marker-specific signals differ by >20%; enumerated marker-specific signals differ by >50%; marker-specific signal from a given disease sample is >85%; >90%; >95%; >96%; >97%; or >98% of the same marker-specific signals from a set of normal samples; or marker-specific signal from a given disease sample has a z-score of >1.03; >1.28; >1.65; >1.75; >1.88; or >2.05 compared to the same marker-specific signals from a set of normal samples.

49. A two-step method of diagnosing or prognosing a disease state of cells or tissue based on identifying the presence or level of a plurality of disease-specific and/or cell/tissue-specific DNA, RNA, and/or protein markers in a biological sample of an individual, said two-step method comprising:
obtaining a biological sample, the biological sample including exosomes, tumor-associated vesicles, markers within other protected states, cell-free DNA, RNA, and/or protein originating from the potentially disease state cells or tissue and from one or more other tissues or cells, wherein the biological sample is selected from the group consisting of cells, serum, blood, plasma, amniotic fluid, sputum, urine, bodily fluids, bodily secretions, and bodily excretions, or fractions thereof;
applying a first step to the biological samples with an overall sensitivity of >80% and an overall specificity of >90% or an overall Z-score of >1.28 to identify individuals more likely to be diagnosed or prognosed with the disease state; and
applying a second step to biological samples from those individuals identified in the first step with an overall specificity of >95% or an overall Z-score of >1.65 to diagnose or prognose individuals with the disease state, wherein said applying the first step and/or said applying the second step is carried out using the method of one of claims 41-44.

50. The method of any one of claims 41 through 49, wherein the disease state is a solid tissue cancer including colorectal adenocarcinoma, stomach adenocarcinoma, esophageal carcinoma, breast lobular and ductal carcinoma, uterine corpus endometrial carcinoma, ovarian serous cystadenocarcinoma, cervical squamous cell carcinoma and adenocarcinoma, uterine carcinosarcoma, lung adenocarcinoma, lung squamous cell carcinoma, head & neck squamous cell carcinoma, prostate adenocarcinoma, invasive urothelial bladder cancer, liver hepatoceullular carcinoma, pancreatic ductal adenocarcinoma, or gallbladder adenocarcinoma, wherein at least 50% of the markers in a set each comprise one or more methylated cytosine residues of a CpG sequence, or the complement of one or more methylated cytosine residues of a CpG sequence selected from the list in FIG. 56.

51. The method of one of claims 41 through 49, wherein the disease state is a solid tissue cancer including colorectal adenocarcinoma, stomach adenocarcinoma, esophageal carcinoma, breast lobular and ductal carcinoma, uterine corpus endometrial carcinoma, ovarian serous cystadenocarcinoma, cervical squamous cell carcinoma and adenocarcinoma, uterine carcinosarcoma, lung adenocarcinoma, lung squamous cell carcinoma, head & neck squamous cell carcinoma, prostate adenocarcinoma, invasive urothelial bladder cancer, liver hepatoceullular carcinoma, pancreatic ductal adenocarcinoma, or gallbladder adenocarcinoma, wherein at least 50% of the markers in a set each comprise of one or more methylated residues of one or more chromosomal sub-regions selected from the list in FIG. 57.

52. The method of one of claims 41 through 49, wherein the disease state is a solid tissue cancer including colorectal adenocarcinoma, stomach adenocarcinoma, esophageal carcinoma, breast lobular and ductal carcinoma, uterine corpus endometrial carcinoma, ovarian serous cystadenocarcinoma, cervical squamous cell carcinoma and adenocarcinoma, uterine carcinosarcoma, lung adenocarcinoma, lung squamous cell carcinoma, head & neck squamous cell carcinoma, prostate adenocarcinoma, invasive urothelial bladder cancer, liver hepatoceullular carcinoma, pancreatic ductal adenocarcinoma, or gallbladder adenocarcinoma, wherein the one or more markers in a set comprise of one or more miRNA sequences (mir ID, Gene ID) selected from the group consisting of: hsa-mir-21, MIR21; hsa-mir-182, MIR182; hsa-mir-454, MIR454; hsa-mir-96, MIR96; hsa-mir-183, MIR183; hsa-mir-549, MIR549; hsa-mir-301^{a}, MIR301A; hsa-mir-548f-1, MIR548F1; hsa-mir-301b, MIR301B; hsa-mir-103-1, MIR1031; hsa-mir-18^{a}, MIR18A; hsa-mir-147b, MIR147B; hsa-mir-4326, MIR4326; and hsa-mir-573, MIR573, or one or more lncRNA or ncRNA sequences selected from the list in FIG. 53.

53. The method of one of claims 41 through 49, wherein the disease state is a solid tissue cancer including colorectal adenocarcinoma, stomach adenocarcinoma, esophageal carcinoma, breast lobular and ductal carcinoma, uterine corpus endometrial carcinoma, ovarian serous cystadenocarcinoma, cervical squamous cell carcinoma and adenocarcinoma, uterine carcinosarcoma, lung adenocarcinoma, lung squamous cell carcinoma, head & neck squamous cell carcinoma, prostate adenocarcinoma, invasive urothelial bladder cancer, liver hepatoceullular carcinoma, pancreatic ductal adenocarcinoma, or gallbladder adenocarcinoma, wherein the one or more markers in a set comprise of one or more Exon RNA sequences selected from the list in FIG. 54.

54. The method of one of claims 41 through 49, wherein the disease state is a solid tissue cancer including colorectal adenocarcinoma, stomach adenocarcinoma, esophageal carcinoma, breast lobular and ductal carcinoma, uterine corpus endometrial carcinoma, ovarian serous cystadenocarcinoma, cervical squamous cell carcinoma and adenocarcinoma, uterine carcinosarcoma, lung adenocarcinoma, lung squamous cell carcinoma, head & neck squamous cell carcinoma, prostate adenocarcinoma, invasive urothelial bladder cancer, liver hepatoceullular carcinoma, pancreatic ductal adenocarcinoma, or gallbladder adenocarcinoma, wherein the one or more markers in a set comprise of one or more mRNA sequences, protein expression levels, protein product concentrations, cytokines, or autoantibody to the protein product selected from the list in FIG. 55 or from the group consisting of (Protein name, UniProt ID): Uncharacterized protein C19orf48, Q6RUI8; Protein FAM72B, Q86X60; Protein FAM72D, Q6L9T8; Hydroxyacylglutathione hydrolase-like protein, Q6PII5; Putative methyltransferase NSUN5, Q96P11; RNA pseudouridylate synthase domain-containing protein 1, Q9UJJ7; Collagen triple helix repeat-containing protein 1, Q96CG8; Interleukin-11. P20809; Stromelysin-2, P09238; Matrix metalloproteinase-9, P14780, Podocan-like protein 1, Q6PEZ8; Putative peptide YY-2, Q9NRI6; Osteopontin, P10451; Sulfhydryl oxidase 2, Q6ZRP7; Glypican-2, Q8N158; Macrophage migration inhibitory factor, P14174; Peptidyl-prolyl cis-trans isomerase A, P62937; and Calreticulin, P27797.

55. The method of one of claims 41 through 49, wherein the disease state is a solid tissue cancer including colorectal adenocarcinoma, stomach adenocarcinoma, esophageal carcinoma, breast lobular and ductal carcinoma, uterine corpus endometrial carcinoma, ovarian serous cystadenocarcinoma, cervical squamous cell carcinoma and adenocarcinoma, uterine carcinosarcoma, lung adenocarcinoma, lung squamous cell carcinoma, head & neck squamous cell carcinoma, prostate adenocarcinoma, invasive urothelial bladder cancer, liver hepatoceullular carcinoma, pancreatic ductal adenocarcinoma, or gallbladder adenocarcinoma, wherein the one or more markers in a set comprise of one or more mutations, insertions, deletions, copy number changes, or expression changes in a gene selected from the group consisting of TP53 (tumor protein p53), TTN (titin), MUC16 (mucin 16), and KRAS (Ki-ras2 Kirsten rat sarcoma viral oncogene homolog).

56. The method of one of claims 41 through 49, wherein the disease state is colon adenocarcinoma, rectal adenocarcinoma, stomach adenocarcinoma, or esophageal carcinoma, wherein at least 50% of the markers in a set each comprise one or more methylated cytosine residues of a CpG sequence, or the complement of one or more methylated cytosine residues of a CpG sequence selected from the list in FIG. 44 or in FIG. 59.

57. The method of one of claims 41 through 49, wherein the disease state is colon adenocarcinoma, rectal adenocarcinoma, stomach adenocarcinoma, or esophageal carcinoma, wherein at least 50% of the markers in a set each comprise of one or more methylated residues of one or more chromosomal sub-regions selected from the list in FIG. 45 or in FIG. 60.

58. The method of one of claims 41 through 49, wherein the disease state is colon adenocarcinoma, rectal adenocarcinoma, stomach adenocarcinoma, or esophageal carcinoma, wherein the one or more markers in a set comprise of one or more miRNA sequences selected from the list in FIG. 39; hsa-mir-624, MIR624; or one or more lncRNA or ncRNA sequences selected from the list in FIG. 40 or the group consisting of [Gene ID, Coordinate (GRCh38)]: ENSEMBL ID: LINC01558, chr6:167784537-167796859, and ENSG00000146521.8.

59. The method of one of claims 41 through 49, wherein the disease state is colon adenocarcinoma, rectal adenocarcinoma, stomach adenocarcinoma, or esophageal carcinoma, wherein the one or more markers in a set comprise of one or more Exon RNA sequences selected from the list in FIG. 41 or in FIG. 58.

60. The method of one of claims 41 through 49, wherein the disease state is colon adenocarcinoma, rectal adenocarcinoma, stomach adenocarcinoma, or esophageal carcinoma, wherein the one or more markers in a set comprise of one or more mRNA sequences, protein expression levels, protein product concentrations, cytokines, or autoantibody to the protein product selected from the list in FIG. 42, FIG. 43, or from the group consisting of (Gene Symbol, Chromosome Band, Gene Title, UniProt ID): SELE, 1q22-q25, selectin E, P16581; OTUD4, 4q31.21, OTU domain containing 4, Q01804; BPI, 20q11.23, bactericidal/permeability-increasing protein, P17213; ASB4, 7q21-q22, ankyrin repeat and SOCS box containing 4, Q9Y574; C6orf123, 6q27, chromosome 6 open reading frame 123, Q9Y6Z2; KPNA3, 13q14.3, karyopherin alpha 3 (importin alpha 4), O00505; and NUP98, 11p15, nucleoporin 98 kDa, P52948; or (Protein name, UniProt ID) Bactericidal permeability-increasing protein (BPI) (CAP 57), P17213.

61. The method of one of claims 41 through 49, wherein the disease state is colon adenocarcinoma, rectal adenocarcinoma, stomach adenocarcinoma, or esophageal carcinoma, wherein the one or more markers in a set comprise of one or more mutations, insertions, deletions, copy number changes, or expression changes in a gene selected from the group consisting of APC (APC regulator of WNT signaling pathway), ATM (ATM serine/threonine kinase), CSMD1 (CUB and Sushi multiple domains 1), DNAH11 (dynein axonemal heavy chain 11), DST (dystonin), EP400 (E1A binding protein p400), FAT3 (FAT atypical cadherin 3), FAT4 (FAT atypical cadherin 4), FLG (filaggrin), GLI3 (GLI family zinc finger 3), KRAS (Ki-ras2 Kirsten rat sarcoma viral oncogene homolog), LRP1B (LDL receptor related protein 1B), MUC16 (mucin 16, cell surface associated), OBSCN (obscurin, cytoskeletal calmodulin and titin-interacting RhoGEF), PCLO (piccolo presynaptic cytomatrix protein), PIK3CA (phosphatidylinositol-4,5-bisphosphate 3-kinase catalytic subunit alpha), RYR2 (ryanodine receptor 2), SYNE1 (spectrin repeat containing nuclear envelope protein 1), TP53 (tumor protein p53), TTN (titin), and UNC13C (unc-13 homolog C).

62. The method of one of claims 41 through 49, wherein the disease state is breast lobular and ductal carcinoma, uterine corpus endometrial carcinoma, ovarian serous cystadenocarcinoma, cervical squamous cell carcinoma and adenocarcinoma, or uterine carcinosarcoma, wherein at least 50% of the markers in a set each comprise one or more methylated cytosine residues of a CpG sequence, or the complement of one or more methylated cytosine residues of a CpG sequence selected from the list in FIG. 61.

63. The method of one of claims 41 through 49, wherein the disease state is breast lobular and ductal carcinoma, uterine corpus endometrial carcinoma, ovarian serous cystadenocarcinoma, cervical squamous cell carcinoma and adenocarcinoma, or uterine carcinosarcoma, wherein at least 50% of the markers in a set each comprise of one or more methylated residues of one or more chromosomal sub-regions selected from the list in FIG. 62.

64. The method of one of claims 41 through 49, wherein the disease state is breast lobular and ductal carcinoma, uterine corpus endometrial carcinoma, ovarian serous cystadenocarcinoma, cervical squamous cell carcinoma and adenocarcinoma, or uterine carcinosarcoma, wherein the one or more markers in a set comprise of one or more miRNA sequences selected from the group consisting of (mir ID, Gene ID): hsa-mir-1265, MIR1265.

65. The method of one of claims 41 through 49, wherein the disease state is breast lobular and ductal carcinoma, uterine corpus endometrial carcinoma, ovarian serous cystadenocarcinoma, cervical squamous cell carcinoma and adenocarcinoma, or uterine carcinosarcoma, wherein the one or more markers in a set comprise of one or more Exon RNA sequences (Exon location, Gene) selected from the group consisting of: chr2:179209013-179209087:+, OSBPL6; chr2:179251788-179251866:+, OSBPL6; and chr2:179253736-179253880:+, OSBPL6.

66. The method of one of claims 41 through 49, wherein the disease state is breast lobular and ductal carcinoma, uterine corpus endometrial carcinoma, ovarian serous cystadenocarcinoma, cervical squamous cell carcinoma and adenocarcinoma, or uterine carcinosarcoma, wherein the one or more markers in a set comprise of one or more mRNA sequences, protein expression levels, protein product concentrations, cytokines, or autoantibody to the protein product selected from the group consisting of (Gene Symbol, Chromosome Band, Gene Title, UniProt ID): RSPO2, 8q23.1, R-spondin 2, Q6UXX9; KLC4, 6p21.1, kinesin light chain 4, Q9NSK0; and GLRX, 5q14, glutaredoxin (thioltransferase), P35754; or (Protein name, UniProt ID) R-spondin-2 (Roof plate-specific spondin-2) (hRspo2), Q6UXX9.

67. The method of one of claims 41 through 49, wherein the disease state is breast lobular and ductal carcinoma, uterine corpus endometrial carcinoma, ovarian serous cystadenocarcinoma, cervical squamous cell carcinoma and adenocarcinoma, or uterine carcinosarcoma, wherein the one or more markers in a set comprise of one or more mutations, insertions, deletions, copy number changes, or expression changes in a gene selected from the group consisting of PIK3CA (phosphatidylinositol-4,5-bisphosphate 3-kinase catalytic subunit alpha), and TTN (titin).

68. The method of one of claims 41 through 49, wherein the disease state is lung adenocarcinoma, lung squamous cell carcinoma, or head & neck squamous cell carcinoma, wherein at least 50% of the markers in a set each comprise one or more methylated cytosine residues of a CpG sequence, or the complement of one or more methylated cytosine residues of a CpG sequence selected from the list in FIG. 63.

69. The method of one of claims 41 through 49, wherein the disease state is lung adenocarcinoma, lung squamous cell carcinoma, or head & neck squamous cell carcinoma, wherein at least 50% of the markers in a set each comprise of one or more methylated residues of one or more chromosomal sub-regions selected from the list in FIG. 64.

70. The method of one of claims 41 through 49, wherein the disease state is lung adenocarcinoma, lung squamous cell carcinoma, or head & neck squamous cell carcinoma, wherein the one or more markers in a set comprise of one or more miRNA sequences selected from (mir ID, Gene ID): hsa-mir-28, MIR28.

71. The method of one of claims 41 through 49, wherein the disease state is lung adenocarcinoma, lung squamous cell carcinoma, or head & neck squamous cell carcinoma, wherein the one or more markers in a set comprise of one or more Exon RNA sequences (Exon location, Gene) selected from the group consisting of: chr2: chr1:93307721-93309752:−, FAM69A; chr1:93312740-93312916:−, FAM69A; chr1:93316405-93316512:−, FAM69A; chr1:93341853-93342152:−, FAM69A; chr1:93426933-93427079:−, FAM69A; chr7:40221554-40221627:+, C7orf10; chr7:40234539-40234659:+, C7orf10; chr8:22265823-22266009:+, SLC39A14; chr8:22272293-22272415:+, SLC39A14; chr14:39509936-39510091:−, SEC23A; and chr14:39511990-39512076:−, SEC23A.

72. The method of one of claims 41 through 49, wherein the disease state is lung adenocarcinoma, lung squamous cell carcinoma, or head & neck squamous cell carcinoma, wherein the one or more markers in a set comprise of one or more mRNA sequences, protein expression levels, protein product concentrations, cytokines, or autoantibody to the protein product selected from the group consisting of (Gene Symbol, Chromosome Band, Gene Title, UniProt ID): STRN3, 14q13-q21, striatin, calmodulin binding protein 3, Q13033; LRRC17, 7q22.1, leucine rich repeat containing 17, Q8N6Y2; FAM69A, 1p22, family with sequence similarity 69, member A, Q5T7M9; ATF2, 2q32, activating transcription factor 2, P15336; BHMT 5q14.1, betaine-homocysteine S-methyltransferase, Q93088; ODZ3/TENM3, 4q34.3-q35.1, teneurin transmembrane protein 3, Q9P273; and ZFHX4, 8q21.11, zinc finger homeobox 4, Q86UP3; or (Protein name, UniProt ID): Leucine-rich repeat-containing protein 17 (p37NB), Q8N6Y2.

73. The method of one of claims 41 through 49, wherein the disease state is lung adenocarcinoma, lung squamous cell carcinoma, or head & neck squamous cell carcinoma, wherein the one or more markers in a set comprise of one or more mutations, insertions, deletions, copy number changes, or expression changes in a gene selected from the group consisting of CSMD3 (CUB and Sushi multiple domains 3), DNAH5 (dynein axonemal heavy chain 5), FAT1 (FAT atypical cadherin 1), FLG (filaggrin), KRAS (Ki-ras2 Kirsten rat sarcoma viral oncogene homolog), LRP1B (LDL receptor related protein 1B), MUC16 (mucin 16, cell surface associated), PCLO (piccolo presynaptic cytomatrix protein), PKHD1L1 (PKHD1 like 1), RELN (reelin), RYR2 (ryanodine receptor 2), SI (sucrase-isomaltase), SYNE1 (spectrin repeat containing nuclear envelope protein 1), TP53 (tumor protein p53), TTN (titin), USH2A (usherin), and XIRP2 (xin actin binding repeat containing 2).

74. The method of one of claims 41 through 49, wherein the disease state is prostate adenocarcinoma or invasive urothelial bladder cancer, wherein at least 50% of the markers in a set each comprise one or more methylated cytosine residues of a CpG sequence, or the complement of one or more methylated cytosine residues of a CpG sequence selected from the list in FIG. 65.

75. The method of one of claims 41 through 49, wherein the disease state is prostate adenocarcinoma or invasive urothelial bladder cancer, wherein at least 50% of the markers in a set each comprise of one or more methylated residues of one or more chromosomal sub-regions selected from the list in FIG. 66.

76. The method of one of claims 41 through 49, wherein the disease state is prostate adenocarcinoma or invasive urothelial bladder cancer, wherein the one or more markers in a set comprise of one or more miRNA sequences selected from the group consisting of (mir ID, Gene ID): hsa-mir-491, MIR491; and hsa-mir-1468, MIR1468, or one or more lncRNA or ncRNA sequences selected from the group consisting of [Gene ID, Coordinate (GRCh38), ENSEMBL ID]: AC007383.3, chr2:206084605-206086564, ENSG00000227946.1; and LINC00324, chr17:8220642-8224043, ENSG00000178977.3.

77. The method of one of claims 41 through 49, wherein the disease state is prostate adenocarcinoma or invasive urothelial bladder cancer, wherein the one or more markers in a set comprise of one or more Exon RNA sequences selected from (Exon location, Gene); chr21:45555942-45556055:+, C21orf33.

78. The method of one of claims 41 through 49, wherein the disease state is prostate adenocarcinoma or invasive urothelial bladder cancer, wherein the one or more markers in a set comprise of one or more mRNA sequences, protein expression levels, protein product concentrations, cytokines, or autoantibody to the protein product selected from (Gene Symbol, Chromosome Band, Gene Title, UniProt ID): PMM1, 22q13, phosphomannomutase 1, Q92871.

79. The method of one of claims 41 through 49, wherein the disease state is prostate adenocarcinoma or invasive urothelial bladder cancer, wherein the one or more markers in a set comprise of one or more mutations, insertions, deletions, copy number changes, or expression changes in a gene selected from the group consisting of BAGE2 (BAGE family member 2), DNM1P47 (dynamin 1 pseudogene 47), FRG1BP (region gene 1 family member B, pseudogene), KRAS (Ki-ras2 Kirsten rat sarcoma viral oncogene homolog), RP11-156P1.3, TTN (titin), and TUBB8P7 (tubulin beta 8 class VIII pseudogene 7).

80. The method of one of claims 41 through 49, wherein the disease state is liver hepatoceullular carcinoma, pancreatic ductal adenocarcinoma, or gallbladder adenocarcinoma, wherein at least 50% of the markers in a set each comprise one or more methylated cytosine residues of a CpG sequence, or the complement of one or more methylated cytosine residues of a CpG sequence selected from the list in FIG. 70.

81. The method of one of claims 41 through 49, wherein the disease state is liver hepatoceullular carcinoma, pancreatic ductal adenocarcinoma, or gallbladder adenocarcinoma, wherein at least 50% of the markers in a set each comprise of one or more methylated residues of one or more chromosomal sub-regions selected from the list in FIG. 71.

82. The method of one of claims 41 through 49, wherein the disease state is liver hepatoceullular carcinoma, pancreatic ductal adenocarcinoma, or gallbladder adenocarcinoma, wherein the one or more markers in a set comprise of one or more miRNA sequences selected from (mir ID, Gene ID): hsa-mir-132, MIR132, or one or more lncRNA or ncRNA sequences selected from the list in FIG. 67.

83. The method of one of claims 41 through 49, wherein the disease state is liver hepatoceullular carcinoma, pancreatic ductal adenocarcinoma, or gallbladder adenocarcinoma, wherein the one or more markers in a set comprise of one or more Exon RNA sequences selected from the list in FIG. 68.

84. The method of one of claims 41 through 49, wherein the disease state is liver hepatoceullular carcinoma, pancreatic ductal adenocarcinoma, or gallbladder adenocarcinoma, wherein the one or more markers in a set comprise of one or more mRNA sequences, protein expression levels, protein product concentrations, cytokines, or autoantibody to the protein product selected from the list in FIG. 69 or from the group consisting of (Protein name, UniProt ID); Gelsolin (AGEL) (Actin-depolymerizing factor) (ADF) (Brevin), P06396; Pro-neuregulin-2, O14511; CD59 glycoprotein (1F5 antigen) (20 kDa homologous restriction factor) (HRF-20) (HRF20) (MAC-inhibitory protein) (MAC-IP) (MEM43 antigen) (Membrane attack complex inhibition factor) (MACIF) (Membrane inhibitor of reactive lysis) (MIRL) (Protectin) (CD antigen CD59), P13987; and Divergent protein kinase domain 2B (Deleted in autism-related protein 1), Q9H7Y0.

85. The method of one of claims 41 through 49, wherein the disease state is liver hepatoceullular carcinoma, pancreatic ductal adenocarcinoma, or gallbladder adenocarcinoma, wherein the one or more markers in a set comprise of one or more mutations, insertions, deletions, copy number changes, or expression changes in a gene selected from the group consisting of KRAS (Ki-ras2 Kirsten rat sarcoma viral oncogene homolog), MUC16 (mucin 16, cell surface associated), MUC4 (mucin 4, cell surface associated), TP53 (tumor protein p53), and TTN (titin).
